SEQUENCE LISTING (790 sequences)

SEQ ID NO: 1 - protein, 5 aa, Artificial Sequence
  Description of Artificial Sequence: Synthetic peptide
  LP7A5 HCDR1
DHYIN

SEQ ID NO: 2 - protein, 17 aa, Artificial Sequence
  Description of Artificial Sequence: Synthetic peptide
  LP7A5 HCDR2
QIYPGDGNTYYNQKFKG

SEQ ID NO: 3 - protein, 9 aa, Artificial Sequence
  Description of Artificial Sequence: Synthetic peptide
  LP7A5_1 HCDR3
NYGDYTIDF

SEQ ID NO: 4 - protein, 17 aa, Artificial Sequence
  Description of Artificial Sequence: Synthetic peptide
  LP7A5 LCDR1
KSSQSLLYSSNQKNYLA

SEQ ID NO: 5 - protein, 7 aa, Artificial Sequence
  Description of Artificial Sequence: Synthetic peptide
  LP7A5 LCDR2
WASTRES

SEQ ID NO: 6 - protein, 8 aa, Artificial Sequence
  Description of Artificial Sequence: Synthetic peptide
  LP7A5 LCDR3
QQYYRYHT

SEQ ID NO: 7 - protein, 118 aa, Artificial Sequence
  Description of Artificial Sequence: Synthetic polypeptide
  LP7A5 Heavy Chain Variable Region
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSS

SEQ ID NO: 8 - protein, 112 aa, Artificial Sequence
  Description of Artificial Sequence: Synthetic polypeptide
  LP7A5 Light Chain Variable Region
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIK

SEQ ID NO: 9 - protein, 5 aa, Artificial Sequence
  Description of Artificial Sequence: Synthetic peptide
  I3RB217 HCDR1
SYWIS

SEQ ID NO: 10 - protein, 17 aa, Artificial Sequence
  Description of Artificial Sequence: Synthetic peptide
  I3RB217 HCDR2
IIDPSDSDTRYSPSFQG

SEQ ID NO: 11 - protein, 9 aa, Artificial Sequence
  Description of Artificial Sequence: Synthetic peptide
  I3RB217 HCDR3
GDGSTDLDY

SEQ ID NO: 12 - protein, 11 aa, Artificial Sequence
  Description of Artificial Sequence: Synthetic peptide
  I3RB217 LCDR1
RASQSVSSSYL

SEQ ID NO: 13 - protein, 7 aa, Artificial Sequence
  Description of Artificial Sequence: Synthetic peptide
  I3RB217 LCDR2
GASSRAT

SEQ ID NO: 14 - protein, 9 aa, Artificial Sequence
  Description of Artificial Sequence: Synthetic peptide
  I3RB217 LCDR3
QQDYGFPWT

SEQ ID NO: 15 - protein, 118 aa, Artificial Sequence
  Description of Artificial Sequence: Synthetic polypeptide
  I3RB217 Heavy Chain Variable Region
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSS

SEQ ID NO: 16 - protein, 108 aa, Artificial Sequence
  Description of Artificial Sequence: Synthetic polypeptide
  I3RB217 Light Chain Variable Region
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIK

SEQ ID NO: 17 - protein, 717 aa, Artificial Sequence
  Description of Artificial Sequence: Synthetic polypeptide
  Vgamma9 half antibody
MAWVWTLLFLMAAAQSIQADIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSEVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 18 - protein, 713 aa, Artificial Sequence
  Description of Artificial Sequence: Synthetic polypeptide
  CD123 half antibody
MAWVWTLLFLMAAAQSIQAEIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSEVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 713 aa, Artificial Sequence; identical to SEQ ID NO: 18
  Description of Artificial Sequence: Synthetic polypeptide
  B23B49 half antibody

SEQ ID NO: 20 - DNA, 2151 bp, Artificial Sequence
  Description of Artificial Sequence: Synthetic polynucleotide
  Vgamma9 half antibody
atggcctgggtgtggaccctgctgttcctgatggccgccgcccagagcatccaggccgacatcgtgatgagccagagcccaagcagcctggccgtgagcgtgggcgagaaggtgaccatgagctgcaagagcagccagagcctgctgtacagcagcaaccagaagaactacctggcctggtaccagcagaagccaggccagagcccaaagctgctgatctactgggccagcacccgcgagagcggcgtgccagaccgcttcaccggcagcggcagcggcaccgacttcaccctgaccatcagcagcgtgaaggccgaggacctggccgtgtactactgccagcagtactaccgctaccacaccttcggcaccggcaccaagctggagatcaagcgcaccgtggccgccccaagcgtgttcatcttcccaccaagcgacgagcagctgaagagcggcaccgccagcgtggtgtgcctgctgaacaacttctacccacgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagcccagtgaccaagagcttcaaccgcggcgagtgcggcggcagcgagggcaagagcagcggcagcggcagcgagagcaagagcaccgagggcaagagcagcggcagcggcagcgagagcaagagcaccggcggcagcgaggtgcagctgcagcagagcggcgccgagctggcccgcccaggcgccagcgtgaagctgagctgcaaggccagcggcttcaccttcaccgaccactacatcaactgggtgaagcagcgcaccggccagggcctggagtggatcggccagatctacccaggcgacggcaacacctactacaaccagaagttcaagggcaaggccaccctgaccgccgacaagagcagcagcaccgcctacatgcagctgagcagcctgaccagcgaggacagcgccgtgtacttctgcgccccaaactacggcgactacaccatcgacttctggggccagggcaccagcgtgaccgtgagcagcgccagcaccaagggcccaagcgtgttcccactggccccatgcagccgcagcaccagcgagagcaccgccgccctgggctgcctggtgaaggactacttcccagagccagtgaccgtgagctggaacagcggcgccctgaccagcggcgtgcacaccttcccagccgtgctgcagagcagcggcctgtacagcctgagcagcgtggtgaccgtgccaagcagcagcctgggcaccaagacctacacctgcaacgtggaccacaagccaagcaacaccaaggtggacaagcgcgtggagagcaagtacggcccaccatgcccaccatgcccagccccagaggccgccggcggcccaagcgtgttcctgttcccaccaaagccaaaggacaccctgatgatcagccgcaccccagaggtgacctgcgtggtggtggacgtgagccaggaggacccagaggtgcagttcaactggtacgtggacggcgtggaggtgcacaacgccaagaccaagccacgcgaggagcagttcaacagcacctaccgcgtggtgagcgtgctgaccgtgctgcaccaggactggctgaacggcaaggagtacaagtgcaaggtgagcaacaagggcctgccaagcagcatcgagaagaccatcagcaaggccaagggccagccacgcgagccacaggtgtacaccctgccaccaagccaggaggagatgaccaagaaccaggtgagcctgtggtgcctggtgaagggcttctacccaagcgacatcgccgtggagtgggagagcaacggccagccagagaacaactacaagaccaccccaccagtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaag

SEQ ID NO: 21 - DNA, 2139 bp, Artificial Sequence
  Description of Artificial Sequence: Synthetic polynucleotide
  CD123 half antibody
atggcctgggtgtggaccctgctgttcctgatggccgccgcccagagcatccaggccgagatcgtgctgacccagagcccaggcaccctgagcctgagcccaggcgagcgcgccaccctgagctgccgcgccagccagagcgtgagcagcagctacctggcctggtaccagcagaagccaggccaggccccacgcctgctgatctacggcgccagcagccgcgccaccggcatcccagaccgcttcagcggcagcggcagcggcaccgacttcaccctgaccatcagccgcctggagccagaggacttcgccgtgtactactgccagcaggactacggcttcccatggaccttcggccagggcaccaaggtggagatcaagcgcaccgtggccgccccaagcgtgttcatcttcccaccaagcgacgagcagctgaagagcggcaccgccagcgtggtgtgcctgctgaacaacttctacccacgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagcccagtgaccaagagcttcaaccgcggcgagtgcggcggcagcgagggcaagagcagcggcagcggcagcgagagcaagagcaccgagggcaagagcagcggcagcggcagcgagagcaagagcaccggcggcagcgaggtgcagctggtgcagagcggcgccgaggtgaagaagccaggcgagagcctgaagatcagctgcaagggcagcggctacagcttcaccagctactggatcagctgggtgcgccagatgccaggcaagggcctggagtggatgggcatcatcgacccaagcgacagcgacacccgctacagcccaagcttccagggccaggtgaccatcagcgccgacaagagcatcagcaccgcctacctgcagtggagcagcctgaaggccagcgacaccgccatgtactactgcgcccgcggcgacggcagcaccgacctggactactggggccagggcaccctggtgaccgtgagcagcgccagcaccaagggcccaagcgtgttcccactggccccatgcagccgcagcaccagcgagagcaccgccgccctgggctgcctggtgaaggactacttcccagagccagtgaccgtgagctggaacagcggcgccctgaccagcggcgtgcacaccttcccagccgtgctgcagagcagcggcctgtacagcctgagcagcgtggtgaccgtgccaagcagcagcctgggcaccaagacctacacctgcaacgtggaccacaagccaagcaacaccaaggtggacaagcgcgtggagagcaagtacggcccaccatgcccaccatgcccagccccagaggccgccggcggcccaagcgtgttcctgttcccaccaaagccaaaggacaccctgatgatcagccgcaccccagaggtgacctgcgtggtggtggacgtgagccaggaggacccagaggtgcagttcaactggtacgtggacggcgtggaggtgcacaacgccaagaccaagccacgcgaggagcagttcaacagcacctaccgcgtggtgagcgtgctgaccgtgctgcaccaggactggctgaacggcaaggagtacaagtgcaaggtgagcaacaagggcctgccaagcagcatcgagaagaccatcagcaaggccaagggccagccacgcgagccacaggtgtacaccctgccaccaagccaggaggagatgaccaagaaccaggtgagcctgtggtgcctggtgaagggcttctacccaagcgacatcgccgtggagtgggagagcaacggccagccagagaacaactacaagaccaccccaccagtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaag

SEQ ID NO: 22 - DNA, 2154 bp, Artificial Sequence
  Description of Artificial Sequence: Synthetic polynucleotide
  B23B49 half antibody
atggcctgggtgtggaccctgctgttcctgatggccgccgcccagagcatccaggccgacatcgtgatgacccagagcccagacagcctggccgtgagcctgggcgagcgcgccaccatcaactgccgcgccagccagagcgtggactacaacggcatcagctacatgcactggtaccagcagaagccaggccagccaccaaagctgctgatctacgccgccagcaacccagagagcggcgtgccagaccgcttcagcggcagcggcagcggcaccgacttcaccctgaccatcagcagcctgcaggccgaggacgtggccgtgtactactgccagcagatcatcgaggacccatggaccttcggccagggcaccaaggtggagatcaagcgcaccgtggccgccccaagcgtgttcatcttcccaccaagcgacgagcagctgaagagcggcaccgccagcgtggtgtgcctgctgaacaacttctacccacgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagcccagtgaccaagagcttcaaccgcggcgagtgcggcggcagcgagggcaagagcagcggcagcggcagcgagagcaagagcaccgagggcaagagcagcggcagcggcagcgagagcaagagcaccggcggcagccagatcaccctgaaggagagcggcccaaccctggtgaagccaacccagaccctgaccctgacctgcaccttcagcggcttcagcctgagcaccagcggcatgggcgtgagctggatccgccagccaccaggcaaggccctggagtggctggcccacatctactgggacgacgacaagcgctacaacccaagcctgaagagccgcctgaccatcaccaaggacaccagcaagaaccaggtggtgctgaccatgaccaacatggacccagtggacaccgccacctactactgcgcccgcctgtacggcttcacctacggcttcgcctactggggccagggcaccctggtgaccgtgagcagcgccagcaccaagggcccaagcgtgttcccactggccccatgcagccgcagcaccagcgagagcaccgccgccctgggctgcctggtgaaggactacttcccagagccagtgaccgtgagctggaacagcggcgccctgaccagcggcgtgcacaccttcccagccgtgctgcagagcagcggcctgtacagcctgagcagcgtggtgaccgtgccaagcagcagcctgggcaccaagacctacacctgcaacgtggaccacaagccaagcaacaccaaggtggacaagcgcgtggagagcaagtacggcccaccatgcccaccatgcccagccccagaggccgccggcggcccaagcgtgttcctgttcccaccaaagccaaaggacaccctgatgatcagccgcaccccagaggtgacctgcgtggtggtggacgtgagccaggaggacccagaggtgcagttcaactggtacgtggacggcgtggaggtgcacaacgccaagaccaagccacgcgaggagcagttcaacagcacctaccgcgtggtgagcgtgctgaccgtgctgcaccaggactggctgaacggcaaggagtacaagtgcaaggtgagcaacaagggcctgccaagcagcatcgagaagaccatcagcaaggccaagggccagccacgcgagccacaggtgtacaccctgccaccaagccaggaggagatgaccaagaaccaggtgagcctgtggtgcctggtgaagggcttctacccaagcgacatcgccgtggagtgggagagcaacggccagccagagaacaactacaagaccaccccaccagtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaag

SEQ ID NO: 23 - protein, 445 aa, Artificial Sequence
  Description of Artificial Sequence: Synthetic polypeptide
  VG1 heavy chain 1
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 24 - protein, 219 aa, Artificial Sequence
  Description of Artificial Sequence: Synthetic polypeptide
  VG1 light chain 1
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 445 aa, Artificial Sequence
  Description of Artificial Sequence: Synthetic polypeptide
  VG1 heavy chain 2
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 215 aa, Artificial Sequence
  Description of Artificial Sequence: Synthetic polypeptide
  VG1 light chain 2
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 447 aa, Artificial Sequence
  Description of Artificial Sequence: Synthetic polypeptide
  VG3 heavy chain 1
QVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 28 - protein, 219 aa, Artificial Sequence
  Description of Artificial Sequence: Synthetic polypeptide
  VG3 light chain 1
DIQMTQSPSSLSASVGDRVTITCRSSQSLVHSNGNTYLHWYQQKPGKAPKFLIYKVSNRFSGVPSRFSGSGSGTDFTLTISSLQPEDFATYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 447 aa, Artificial Sequence
  Description of Artificial Sequence: Synthetic polypeptide
  VG3 heavy chain 2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 218 aa, Artificial Sequence
  Description of Artificial Sequence: Synthetic polypeptide
  VG3 light chain 2
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 9 aa, Artificial Sequence
  Description of Artificial Sequence: Synthetic peptide
  LP7A5_2 HCDR3
NMGMYTIDF

SEQ ID NO: 32 - protein, 9 aa, Artificial Sequence
  Description of Artificial Sequence: Synthetic peptide
  LP7A5_3 HCDR3
NMGMYTLDF

SEQ ID NO: 33 - protein, 9 aa, Artificial Sequence
  Description of Artificial Sequence: Synthetic peptide
  LP7A5_4 HCDR3
NYGDYTLDF

SEQ ID NO: 34 - protein, 118 aa, Artificial Sequence
  Description of Artificial Sequence: Synthetic polypeptide
  LP7A5_2 Heavy Chain Variable Region
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNMGMYTIDFWGQGTSVTVSS

SEQ ID NO: 35 - protein, 118 aa, Artificial Sequence
  Description of Artificial Sequence: Synthetic polypeptide
  LP7A5_3 Heavy Chain Variable Region
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNMGMYTLDFWGQGTSVTVSS

SEQ ID NO: 36 - protein, 118 aa, Artificial Sequence
  Description of Artificial Sequence: Synthetic polypeptide
  LP7A5_4 Heavy Chain Variable Region
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTLDFWGQGTSVTVSS

SEQ ID NO: 37 - protein, 5 aa, Artificial Sequence
  Description of Artificial Sequence: Synthetic peptide
  HCDR1 Sequence of anti-CD33 mAb of antibody C33B904
DYAMH

SEQ ID NO: 38 - protein, 17 aa, Artificial Sequence
  Description of Artificial Sequence: Synthetic peptide
  HCDR2 Sequence of anti-CD33 mAb of antibody C33B904
GIGWSGGSIVYADSVKG

SEQ ID NO: 39 - protein, 10 aa, Artificial Sequence
  Description of Artificial Sequence: Synthetic peptide
  HCDR3 Sequence of anti-CD33 mAb of antibody C33B904
DSPYGDFFDY

SEQ ID NO: 40 - protein, 17 aa, Artificial Sequence
  Description of Artificial Sequence: Synthetic peptide
  LCDR1 Sequence of anti-CD33 mAb of antibody C33B904
KSSQTVFYSSNNKNYLA

SEQ ID NO: 41 - protein, 7 aa, Artificial Sequence
  Description of Artificial Sequence: Synthetic peptide
  LCDR2 Sequence of anti-CD33 mAb of antibody C33B904
WASTRKS

SEQ ID NO: 42 - protein, 9 aa, Artificial Sequence
  Description of Artificial Sequence: Synthetic peptide
  LCDR3 Sequence of anti-CD33 mAb of antibody C33B904
QHYYSTPYT

SEQ ID NO: 43 - protein, 119 aa, Artificial Sequence
  Description of Artificial Sequence: Synthetic polypeptide
  Heavy chain sequence of anti-CD33 mAb of antibody C33B904
EVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSS

SEQ ID NO: 44 - protein, 113 aa, Artificial Sequence
  Description of Artificial Sequence: Synthetic polypeptide
  Light chain sequence of anti-CD33 mAb of antibody C33B904
DIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIK

SEQ ID NO: 45 - protein, 719 aa, Artificial Sequence
  Description of Artificial Sequence: Synthetic polypeptide
  Anti-CD33 (′knob′-arm) sequence expressed in CHO cells
MAWVWTLLFLMAAAQSIQADIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSEVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 713 aa, Artificial Sequence; identical to SEQ ID NO: 18
  Description of Artificial Sequence: Synthetic polypeptide
  Anti-RSV (′knob′-arm) sequence expressed in CHO cells

SEQ ID NO: 47 - protein, 446 aa, Artificial Sequence
  Description of Artificial Sequence: Synthetic polypeptide
  C33B904 Heavy Chain
EVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 220 aa, Artificial Sequence
  Description of Artificial Sequence: Synthetic polypeptide
  C33B904 Light Chain
DIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 8 aa, Artificial Sequence
  Description of Artificial Sequence: Synthetic peptide
  mJOVI-1 HCDR1
GYTFTGYV

SEQ ID NO: 50 - protein, 8 aa, Artificial Sequence
  Description of Artificial Sequence: Synthetic peptide
  mJOVI-1 HCDR2
INPYNDDI

SEQ ID NO: 51 - protein, 17 aa, Artificial Sequence
  Description of Artificial Sequence: Synthetic peptide
  mJOVI-1 HCDR3
ARGAGYNFDGAYRFFDF

SEQ ID NO: 52 - protein, 11 aa, Artificial Sequence
  Description of Artificial Sequence: Synthetic peptide
  mJOVI-1 LCDR1
QRLVHSNGNTY

SEQ ID NO: 53 - protein, 3 aa, Artificial Sequence
  Description of Artificial Sequence: Synthetic peptide
  mJOVI-1 LCDR2
RVS

SEQ ID NO: 54 - protein, 9 aa, Artificial Sequence
  Description of Artificial Sequence: Synthetic peptide
  mJOVI-1 LCDR3
SQSTHVPYT

SEQ ID NO: 55 - protein, 124 aa, Artificial Sequence
  Description of Artificial Sequence: Synthetic polypeptide
  mJOVI-1 Heavy Chain Variable Region
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSS

SEQ ID NO: 56 - protein, 112 aa, Artificial Sequence
  Description of Artificial Sequence: Synthetic polypeptide
  mJOVI-1 Light Chain Variable Region
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIK

SEQ ID NO: 57 - protein, 507 aa, Artificial Sequence
  Description of Artificial Sequence: Synthetic polypeptide
  TRBC1 Half Ab scFv
MAWVWTLLFLMAAAQSIQADVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKGGSEGKSSGSGSESKSTGGSEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 58 - protein, 454 aa, Artificial Sequence
  Description of Artificial Sequence: Synthetic polypeptide
  TRBC1 Heavy Chain
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNRFTQKSLSLSPGK

SEQ ID NO: 59 - protein, 219 aa, Artificial Sequence
  Description of Artificial Sequence: Synthetic polypeptide
  TRBC1 Light Chain
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 8 aa, Artificial Sequence
  Description of Artificial Sequence: Synthetic peptide
  TRGV9Ab HCDR1
GFTFTDHY

SEQ ID NO: 61 - protein, 8 aa, Artificial Sequence
  Description of Artificial Sequence: Synthetic peptide
  TRGV9Ab HCDR2
IYPGSGNT

SEQ ID NO: 62 - protein, 11 aa, Artificial Sequence
  Description of Artificial Sequence: Synthetic peptide
  TRGV9Ab HCDR3
ARNYGDYTIDF

SEQ ID NO: 63 - protein, 12 aa, Artificial Sequence
  Description of Artificial Sequence: Synthetic peptide
  TRGV9Ab LCDR1
QSVLYSSNNKNY

SEQ ID NO: 64 - protein, 3 aa, Artificial Sequence
  Description of Artificial Sequence: Synthetic peptide
  TRGV9Ab LCDR2
WAS

SEQ ID NO: 65 - protein, 118 aa, Artificial Sequence
  Description of Artificial Sequence: Synthetic polypeptide
  TRGV9Ab_var17 Heavy Chain Variable Region
QVQLVQSGAEVKKPGASVKVSCKASGFTFTDHYINWVRQATGQGLEWMGQIYPGSGNTYYNQKFKGRVTMTRDTSISTAYMELSSLRSEDTAVYYCARNYGDYTIDFWGQGTSVTVSS

SEQ ID NO: 66 - protein, 112 aa, Artificial Sequence
  Description of Artificial Sequence: Synthetic polypeptide
  TRGV9Ab_var17 Light Chain Variable Region
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYRYHTFGTGTKLEIK

SEQ ID NO: 67 - protein, 118 aa, Artificial Sequence
  Description of Artificial Sequence: Synthetic polypeptide
  TRGV9Ab_var29 Heavy Chain Variable Region
QVQLVQSGAEVKKPGASVKVSCKASGFTFTDHYINWVRQATGQGLEWMGQIYPGSGNTYYNQKFKGRVTMTRNTSISTAYMELSSLRSEDTAVYYCARNYGDYTIDFWGQGTSVTVSS

SEQ ID NO: 68 - protein, 112 aa, Artificial Sequence
  Description of Artificial Sequence: Synthetic polypeptide
  TRGV9Ab_var29 Light Chain Variable Region
DIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYRYHTFGTGTKLEIK

SEQ ID NO: 69 - protein, 448 aa, Artificial Sequence
  Description of Artificial Sequence: Synthetic polypeptide
  TRGV9_7A5_1 Heavy  Chain
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 70 - protein, 501 aa, Artificial Sequence
  Description of Artificial Sequence: Synthetic polypeptide
  TRGV9_var17 scFv
MAWVWTLLFLMAAAQSIQADIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYRYHTFGTGTKLEIKGGSEGKSSGSGSESKSTGGSQVQLVQSGAEVKKPGASVKVSCKASGFTFTDHYINWVRQATGQGLEWMGQIYPGSGNTYYNQKFKGRVTMTRDTSISTAYMELSSLRSEDTAVYYCARNYGDYTIDFWGQGTSVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 71 - protein, 448 aa, Artificial Sequence
  Description of Artificial Sequence: Synthetic polypeptide
  TRGV9_var17 Heavy  Chain
QVQLVQSGAEVKKPGASVKVSCKASGFTFTDHYINWVRQATGQGLEWMGQIYPGSGNTYYNQKFKGRVTMTRDTSISTAYMELSSLRSEDTAVYYCARNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 72 - protein, 219 aa, Artificial Sequence
  Description of Artificial Sequence: Synthetic polypeptide
  TRGV9_var17 Light Chain
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 501 aa, Artificial Sequence
  Description of Artificial Sequence: Synthetic polypeptide
  TRGV9_var29 scFv
MAWVWTLLFLMAAAQSIQADIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYRYHTFGTGTKLEIKGGSEGKSSGSGSESKSTGGSQVQLVQSGAEVKKPGASVKVSCKASGFTFTDHYINWVRQATGQGLEWMGQIYPGSGNTYYNQKFKGRVTMTRNTSISTAYMELSSLRSEDTAVYYCARNYGDYTIDFWGQGTSVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 74 - protein, 448 aa, Artificial Sequence
  Description of Artificial Sequence: Synthetic polypeptide
  TRGV9_var29 Heavy Chain
QVQLVQSGAEVKKPGASVKVSCKASGFTFTDHYINWVRQATGQGLEWMGQIYPGSGNTYYNQKFKGRVTMTRNTSISTAYMELSSLRSEDTAVYYCARNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 75 - protein, 219 aa, Artificial Sequence
  Description of Artificial Sequence: Synthetic polypeptide
  TRGV9_var29 Light Chain
DIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 17 aa, Artificial Sequence
  Description of Artificial Sequence: Synthetic peptide
  TRGV9Ab HCDR2
QIYPGSGNTYYNQKFKG

SEQ ID NO: 77 - protein, 17 aa, Artificial Sequence
  Description of Artificial Sequence: Synthetic peptide
  TRGV9Ab LCDR1
KSSQSVLYSSNNKNYLA

SEQ ID NO: 78 - protein, 467 aa, Artificial Sequence
  Description of Artificial Sequence: Synthetic polypeptide
  Anti-TRGV9_7A5_1 (half-mAb) Heavy Chain A
MAWVWTLLFLMAAAQSIQAEVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 79 - protein, 238 aa, Artificial Sequence
  Description of Artificial Sequence: Synthetic polypeptide
  Anti-TRGV9_7A5_1 (half-mAb) Light Chain
MAWVWTLLFLMAAAQSIQADIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 467 aa, Artificial Sequence
  Description of Artificial Sequence: Synthetic polypeptide
  Anti- TRGV9_7A5_var17 Heavy Chain A
MAWVWTLLFLMAAAQSIQAQVQLVQSGAEVKKPGASVKVSCKASGFTFTDHYINWVRQATGQGLEWMGQIYPGSGNTYYNQKFKGRVTMTRDTSISTAYMELSSLRSEDTAVYYCARNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 81 - protein, 238 aa, Artificial Sequence
  Description of Artificial Sequence: Synthetic polypeptide
  Anti- TRGV9_7A5_var17 Light Chain
MAWVWTLLFLMAAAQSIQADIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 467 aa, Artificial Sequence
  Description of Artificial Sequence: Synthetic polypeptide
  Anti- TRGV9_7A5_var29 Heavy Chain A
MAWVWTLLFLMAAAQSIQAQVQLVQSGAEVKKPGASVKVSCKASGFTFTDHYINWVRQATGQGLEWMGQIYPGSGNTYYNQKFKGRVTMTRNTSISTAYMELSSLRSEDTAVYYCARNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 83 - protein, 238 aa, Artificial Sequence
  Description of Artificial Sequence: Synthetic polypeptide
  Anti- TRGV9_7A5_var29 Light Chain
MAWVWTLLFLMAAAQSIQADIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 473 aa, Artificial Sequence
  Description of Artificial Sequence: Synthetic polypeptide
  Anti-TRBC1 (half-mAb) Heavy Chain B
MAWVWTLLFLMAAAQSIQAEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNRFTQKSLSLSPGK

SEQ ID NO: 85 - protein, 238 aa, Artificial Sequence
  Description of Artificial Sequence: Synthetic polypeptide
  Anti-TRBC1 (half-mAb) Light Chain
MAWVWTLLFLMAAAQSIQADVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 469 aa, Artificial Sequence
  Description of Artificial Sequence: Synthetic polypeptide
  Anti-RSV (half-mAb) Heavy Chain B
MAWVWTLLFLMAAAQSIQAQITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 87 - protein, 238 aa, Artificial Sequence
  Description of Artificial Sequence: Synthetic polypeptide
  Anti-RSV (half-mAb) Light Chain
METHSQVFVYMLLWLSGVEGDIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 495 aa, Artificial Sequence
  Description of Artificial Sequence: Synthetic polypeptide
  Anti-Null-scFv Heavy Chain B
MAWVWTLLFLMAAAQSIQADIQMTQSPSSLSASVGDRVTITCRASQSISSYLNWYQQKPGCAPKLLIYAASSLQSGVPSRFSGSGSGTDFTLTISSLQPEDFATYYCQQSYSTPLTFGQGTKVEIKGGGSGGSGGCPPCGGSGGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 89 - protein, 8 aa, Artificial Sequence
  Description of Artificial Sequence: Synthetic peptide
GFTFSSNY

SEQ ID NO: 90 - protein, 8 aa, Artificial Sequence
  Description of Artificial Sequence: Synthetic peptide
IHGGTGGI

SEQ ID NO: 91 - protein, 11 aa, Artificial Sequence
  Description of Artificial Sequence: Synthetic peptide
ARRGYGAWFAY

SEQ ID NO: 92 - protein, 6 aa, Artificial Sequence
  Description of Artificial Sequence: Synthetic peptide
ENIHNY

SEQ ID NO: 93 - protein, 3 aa, Artificial Sequence
  Description of Artificial Sequence: Synthetic peptide
NAK

SEQ ID NO: 94 - protein, 9 aa, Artificial Sequence
  Description of Artificial Sequence: Synthetic peptide
QHFWSYPLT

SEQ ID NO: 95 - protein, 118 aa, Artificial Sequence
  Description of Artificial Sequence: Synthetic polypeptide
QGQMQQSGAELVKPGASVKLSCKTSGFTFSSNYISWLKQKPGQSLEWIAWIHGGTGGIGYNQKFTGKAQLTVDTSSTTAYMQFSSLTTEDSAIYYCARRGYGAWFAYWGQGTLVTVSA

SEQ ID NO: 96 - protein, 107 aa, Artificial Sequence
  Description of Artificial Sequence: Synthetic polypeptide
DIQMTQSPASLSASVGETVTITCRASENIHNYLAWYQQKQGKSPQLLVYNAKTLADGVPSRFSGSGSGTQYSLKINSLQPEDFGNYYCQHFWSYPLTFGAGTKLELK

SEQ ID NO: 97 - protein, 496 aa, Artificial Sequence
  Description of Artificial Sequence: Synthetic polypeptide
MAWVWTLLFLMAAAQSIQADIQMTQSPASLSASVGETVTITCRASENIHNYLAWYQQKQGKSPQLLVYNAKTLADGVPSRFSGSGSGTQYSLKINSLQPEDFGNYYCQHFWSYPLTFGAGTKLELKGGSEGKSSGSGSESKSTGGSQGQMQQSGAELVKPGASVKLSCKTSGFTFSSNYISWLKQKPGQSLEWIAWIHGGTGGIGYNQKFTGKAQLTVDTSSTTAYMQFSSLTTEDSAIYYCARRGYGAWFAYWGQGTLVTVSAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 98 - protein, 8 aa, Artificial Sequence
  Description of Artificial Sequence: Synthetic peptide
GFTFSNYD

SEQ ID NO: 99 - protein, 8 aa, Artificial Sequence
  Description of Artificial Sequence: Synthetic peptide
ISSSSSYI

SEQ ID NO: 100 - protein, 16 aa, Artificial Sequence
  Description of Artificial Sequence: Synthetic peptide
ARDVGVTDYYYYGMDV

SEQ ID NO: 101 - protein, 7 aa, Artificial Sequence
  Description of Artificial Sequence: Synthetic peptide
QSVASSY

SEQ ID NO: 102 - protein, 3 aa, Artificial Sequence
  Description of Artificial Sequence: Synthetic peptide
GAS

SEQ ID NO: 103 - protein, 10 aa, Artificial Sequence
  Description of Artificial Sequence: Synthetic peptide
QQYGSSPPYT

SEQ ID NO: 104 - protein, 124 aa, Artificial Sequence
  Description of Artificial Sequence: Synthetic polypeptide
EVQLVESGGGLVKPGGSLRLSCSASGFTFSNYDMNWVRQAPGKGLEWVSSISSSSSYIYYADSVKGRFTISRDNAKNSLYLQMNSLRAEDTAVYHCARDVGVTTDYYYYGMDVWGQGTMVTVSS

SEQ ID NO: 105 - protein, 109 aa, Artificial Sequence
  Description of Artificial Sequence: Synthetic polypeptide
EIVMTQSPGTLSLSPGDRATLSCRASQSVASSYLAWYQQKPGQSPRLLIYGASSRATGIPDRFSGSGSGTDFTLTISRLEPEDFAVYYCQQYGSSPPYTFGQGTRLEIK

SEQ ID NO: 106 - protein, 504 aa, Artificial Sequence
  Description of Artificial Sequence: Synthetic polypeptide
MAWVWTLLFLMAAAQSIQAEIVMTQSPGTLSLSPGDRATLSCRASQSVASSYLAWYQQKPGQSPRLLIYGASSRATGIPDRFSGSGSGTDFTLTISRLEPEDFAVYYCQQYGSSPPYTFGQGTRLEIKGGSEGKSSGSGSESKSTGGSEVQLVESGGGLVKPGGSLRLSCSASGFTFSNYDMNWVRQAPGKGLEWVSSISSSSSYIYYADSVKGRFTISRDNAKNSLYLQMNSLRAEDTAVYHCARDVGVTTDYYYYGMDVWGQGTMVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 107 - protein, 8 aa, Artificial Sequence
  Description of Artificial Sequence: Synthetic peptide
GDTFNNYA

SEQ ID NO: 108 - protein, 8 aa, Artificial Sequence
  Description of Artificial Sequence: Synthetic peptide
IIPFFGTP

SEQ ID NO: 109 - protein, 17 aa, Artificial Sequence
  Description of Artificial Sequence: Synthetic peptide
ARPGSGSPDYYYYDMDV

SEQ ID NO: 110 - protein, 11 aa, Artificial Sequence
  Description of Artificial Sequence: Synthetic peptide
QSLVHSDGNTY

SEQ ID NO: 111 - protein, 3 aa, Artificial Sequence
  Description of Artificial Sequence: Synthetic peptide
KIS

SEQ ID NO: 112 - protein, 9 aa, Artificial Sequence
  Description of Artificial Sequence: Synthetic peptide
MQATQFPLT

SEQ ID NO: 113 - protein, 124 aa, Artificial Sequence
  Description of Artificial Sequence: Synthetic polypeptide
EVQLVQSGAEVKKPGSSVKVSCKASGDTFNNYAISWVRQAPGQGLEWMGGIIPFFGTPDYAQKFQGRVTITADKSTSTAYMELSGLRSEDTAVYYCARPGSGSPDYYYYDMDVWGQGTTVTVSS

SEQ ID NO: 114 - protein, 112 aa, Artificial Sequence
  Description of Artificial Sequence: Synthetic polypeptide
DIVMTQTPLSSPVTLGQPASISCRSSQSLVHSDGNTYLSWLQQRPGQPPRLLIYKISNRFSGVPDRFSGSGAGTDFTLKINRVEAEDVGVYYCMQATQFPLTFGGGTKVEIK

SEQ ID NO: 115 - protein, 454 aa, Artificial Sequence
  Description of Artificial Sequence: Synthetic polypeptide
EVQLVQSGAEVKKPGSSVKVSCKASGDTFNNYAISWVRQAPGQGLEWMGGIIPFFGTPDYAQKFQGRVTITADKSTSTAYMELSGLRSEDTAVYYCARPGSGSPDYYYYDMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 116 - protein, 219 aa, Artificial Sequence
  Description of Artificial Sequence: Synthetic polypeptide
DIVMTQTPLSSPVTLGQPASISCRSSQSLVHSDGNTYLSWLQQRPGQPPRLLIYKISNRFSGVPDRFSGSGAGTDFTLKINRVEAEDVGVYYCMQATQFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 8 aa, Artificial Sequence
  Description of Artificial Sequence: Synthetic peptide
GGTFSSYA

SEQ ID NO: 118 - protein, 8 aa, Artificial Sequence
  Description of Artificial Sequence: Synthetic peptide
NIPIFNTA

SEQ ID NO: 119 - protein, 13 aa, Artificial Sequence
  Description of Artificial Sequence: Synthetic peptide
VREGTGYSYGLDY

SEQ ID NO: 120 - protein, 11 aa, Artificial Sequence
  Description of Artificial Sequence: Synthetic peptide
QSLIHSDGNTY

SEQ ID NO: 121 - protein, 3 aa, Artificial Sequence
  Description of Artificial Sequence: Synthetic peptide
KIS

SEQ ID NO: 122 - protein, 9 aa, Artificial Sequence
  Description of Artificial Sequence: Synthetic peptide
MQAKQFPIT

SEQ ID NO: 123 - protein, 120 aa, Artificial Sequence
  Description of Artificial Sequence: Synthetic polypeptide
EVQLVQSGAEVKKPGSSVKVSCKASGGTFSSYAISWVRQAPGQGLEWMGGNIPIFNTANYAQKFQDRVTITADKSTSTAYMELSSLRSEDTAVYYCVREGTGYSYGLDYWGQGTPVTVSS

SEQ ID NO: 124 - protein, 112 aa, Artificial Sequence
  Description of Artificial Sequence: Synthetic polypeptide
EIVMTQSPLSSPVTLGQPASISCRSSQSLIHSDGNTYLSWLQQRPGQPPRLLIYKISNRFSGVPDRFSGSGAGTDFTLKISRVEAEDVGIYYCMQAKQFPITFGQGTKVDIK

SEQ ID NO: 125 - protein, 441 aa, Artificial Sequence
  Description of Artificial Sequence: Synthetic polypeptide
EVQLVQSGAEVKKPGSSVKVSCKASGGTFSSYAISWVRQAPGQGLEWMGGNIPIFNTANYAQKFQDRVTITADKSTSTAYMELSSLRSEDTAVYYCVREGTGYSYGLDYWGQGTPVTVSSASTKGPSVTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 126 - protein, 219 aa, Artificial Sequence
  Description of Artificial Sequence: Synthetic polypeptide
EIVMTQSPLSSPVTLGQPASISCRSSQSLIHSDGNTYLSWLQQRPGQPPRLLIYKISNRFSGVPDRFSGSGAGTDFTLKISRVEAEDVGIYYCMQAKQFPITFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 10 aa, Artificial Sequence
  Description of Artificial Sequence: Synthetic peptide
GGSISSGGSY

SEQ ID NO: 128 - protein, 7 aa, Artificial Sequence
  Description of Artificial Sequence: Synthetic peptide
IYNSGST

SEQ ID NO: 129 - protein, 12 aa, Artificial Sequence
  Description of Artificial Sequence: Synthetic peptide
ARDSNYEWFFDL

SEQ ID NO: 130 - protein, 6 aa, Artificial Sequence
  Description of Artificial Sequence: Synthetic peptide
QSVSSY

SEQ ID NO: 131 - protein, 3 aa, Artificial Sequence
  Description of Artificial Sequence: Synthetic peptide
DAS

SEQ ID NO: 132 - protein, 9 aa, Artificial Sequence
  Description of Artificial Sequence: Synthetic peptide
QQRSNWPLT

SEQ ID NO: 133 - protein, 120 aa, Artificial Sequence
  Description of Artificial Sequence: Synthetic polypeptide
QVQLQESGPGLVKPSQTLSLTCTVSGGSISSGGSYWSWIRQHPGKGLEWIGYIYNSGSTYYNPSLKSRVSMSVDTSKNQFSLKLSSVTAADTAVYYCARDSNYEWFFDLWGPGTLVTVSS

SEQ ID NO: 134 - protein, 107 aa, Artificial Sequence
  Description of Artificial Sequence: Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 135 - protein, 450 aa, Artificial Sequence
  Description of Artificial Sequence: Synthetic polypeptide
QVQLQESGPGLVKPSQTLSLTCTVSGGSISSGGSYWSWIRQHPGKGLEWIGYIYNSGSTYYNPSLKSRVSMSVDTSKNQFSLKLSSVTAADTAVYYCARDSNYEWFFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 136 - protein, 214 aa, Artificial Sequence
  Description of Artificial Sequence: Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 8 aa, Artificial Sequence
  Description of Artificial Sequence: Synthetic peptide
GFTFSSYA

SEQ ID NO: 138 - protein, 8 aa, Artificial Sequence
  Description of Artificial Sequence: Synthetic peptide
ISGSGGST

SEQ ID NO: 139 - protein, 16 aa, Artificial Sequence
  Description of Artificial Sequence: Synthetic peptide
AKDEGYSSGHYYGMDV

SEQ ID NO: 140 - protein, 7 aa, Artificial Sequence
  Description of Artificial Sequence: Synthetic peptide
QSISSSF

SEQ ID NO: 141 - protein, 3 aa, Artificial Sequence
  Description of Artificial Sequence: Synthetic peptide
GAS

SEQ ID NO: 142 - protein, 10 aa, Artificial Sequence
  Description of Artificial Sequence: Synthetic peptide
QHYGSSPMYT

SEQ ID NO: 143 - protein, 123 aa, Artificial Sequence
  Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSS

SEQ ID NO: 144 - protein, 109 aa, Artificial Sequence
  Description of Artificial Sequence: Synthetic polypeptide
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIK

SEQ ID NO: 145 - protein, 503 aa, Artificial Sequence
  Description of Artificial Sequence: Synthetic polypeptide
MAWVWTLLFLMAAAQSIQAEIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKGGSEGKSSGSGSESKSTGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 146 - protein, 453 aa, Artificial Sequence
  Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNRFTQKSLSLSPGK

SEQ ID NO: 147 - protein, 216 aa, Artificial Sequence
  Description of Artificial Sequence: Synthetic polypeptide
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 472 aa, Artificial Sequence
  Description of Artificial Sequence: Synthetic polypeptide
MAWVWTLLFLMAAAQSIQAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNRFTQKSLSLSPGK

SEQ ID NO: 149 - protein, 235 aa, Artificial Sequence
  Description of Artificial Sequence: Synthetic polypeptide
MAWVWTLLFLMAAAQSIQAEIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 473 aa, Artificial Sequence
  Description of Artificial Sequence: Synthetic polypeptide
MAWVWTLLFLMAAAQSIQAEVQLVQSGAEVKKPGSSVKVSCKASGDTFNNYAISWVRQAPGQGLEWMGGIIPFFGTPDYAQKFQGRVTITADKSTSTAYMELSGLRSEDTAVYYCARPGSGSPDYYYYDMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 151 - protein, 238 aa, Artificial Sequence
  Description of Artificial Sequence: Synthetic polypeptide
MAWVWTLLFLMAAAQSIQADIVMTQTPLSSPVTLGQPASISCRSSQSLVHSDGNTYLSWLQQRPGQPPRLLIYKISNRFSGVPDRFSGSGAGTDFTLKINRVEAEDVGVYYCMQATQFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 469 aa, Artificial Sequence
  Description of Artificial Sequence: Synthetic polypeptide
MAWVWTLLFLMAAAQSIQAEVQLVQSGAEVKKPGSSVKVSCKASGGTFSSYAISWVRQAPGQGLEWMGGNIPIFNTANYAQKFQDRVTITADKSTSTAYMELSSLRSEDTAVYYCVREGTGYSYGLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 153 - protein, 238 aa, Artificial Sequence
  Description of Artificial Sequence: Synthetic polypeptide
MAWVWTLLFLMAAAQSIQAEIVMTQSPLSSPVTLGQPASISCRSSQSLIHSDGNTYLSWLQQRPGQPPRLLIYKISNRFSGVPDRFSGSGAGTDFTLKISRVEAEDVGIYYCMQAKQFPITFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 469 aa, Artificial Sequence
  Description of Artificial Sequence: Synthetic polypeptide
MAWVWTLLFLMAAAQSIQAQVQLQESGPGLVKPSQTLSLTCTVSGGSISSGGSYWSWIRQHPGKGLEWIGYIYNSGSTYYNPSLKSRVSMSVDTSKNQFSLKLSSVTAADTAVYYCARDSNYEWFFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 155 - protein, 233 aa, Artificial Sequence
  Description of Artificial Sequence: Synthetic polypeptide
MAWVWTLLFLMAAAQSIQAEIVMTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 493 aa, Artificial Sequence
  Description of Artificial Sequence: Synthetic polypeptide
MAWVWTLLFLMAAAQSIQAAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 157 - protein, 480 aa, Artificial Sequence
  Description of Artificial Sequence: Synthetic polypeptide
MAWVWTLLFLMAAAQSIQAAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 158-159 - skipped (2 entries)

SEQ ID NO: 160 - protein, 10 aa, Artificial Sequence
  Description of Artificial Sequence: Synthetic peptide
GFTFTDHYIN

SEQ ID NO: 161 - protein, 17 aa, Artificial Sequence
  Description of Artificial Sequence: Synthetic peptide
QIYPGDGNTYYNQKFKG

SEQ ID NO: 162 - protein, 9 aa, Artificial Sequence
  Description of Artificial Sequence: Synthetic peptide
NYGDYTIDF

SEQ ID NO: 163 - protein, 17 aa, Artificial Sequence
  Description of Artificial Sequence: Synthetic peptide
KSSQSLLYSSNQKNYLA

SEQ ID NO: 164 - protein, 7 aa, Artificial Sequence
  Description of Artificial Sequence: Synthetic peptide
WASTRES

SEQ ID NO: 165 - protein, 8 aa, Artificial Sequence
  Description of Artificial Sequence: Synthetic peptide
QQYYRYHT

SEQ ID NO: 166 - protein, 8 aa, Artificial Sequence
  Description of Artificial Sequence: Synthetic peptide
GFTFTDHY

SEQ ID NO: 167 - protein, 8 aa, Artificial Sequence
  Description of Artificial Sequence: Synthetic peptide
IYPGDGNT

SEQ ID NO: 168 - protein, 11 aa, Artificial Sequence
  Description of Artificial Sequence: Synthetic peptide
APNYGDYTIDF

SEQ ID NO: 169 - protein, 12 aa, Artificial Sequence
  Description of Artificial Sequence: Synthetic peptide
QSLLYSSNQKNY

SEQ ID NO: 170 - protein, 3 aa, Artificial Sequence
  Description of Artificial Sequence: Synthetic peptide
WAS

SEQ ID NO: 171 - protein, 8 aa, Artificial Sequence
  Description of Artificial Sequence: Synthetic peptide
QQYYRYHT

SEQ ID NO: 172 - protein, 5 aa, Artificial Sequence
  Description of Artificial Sequence: Synthetic peptide
DHYIN

SEQ ID NO: 173 - protein, 17 aa, Artificial Sequence
  Description of Artificial Sequence: Synthetic peptide
QIYPGDGNTYYNQKFKG

SEQ ID NO: 174 - protein, 9 aa, Artificial Sequence
  Description of Artificial Sequence: Synthetic peptide
NYGDYTIDF

SEQ ID NO: 175 - protein, 17 aa, Artificial Sequence
  Description of Artificial Sequence: Synthetic peptide
KSSQSLLYSSNQKNYLA

SEQ ID NO: 176 - protein, 7 aa, Artificial Sequence
  Description of Artificial Sequence: Synthetic peptide
WASTRES

SEQ ID NO: 177 - protein, 8 aa, Artificial Sequence
  Description of Artificial Sequence: Synthetic peptide
QQYYRYHT

SEQ ID NO: 178 - protein, 7 aa, Artificial Sequence
  Description of Artificial Sequence: Synthetic peptide
GFTFTDH

SEQ ID NO: 179 - protein, 4 aa, Artificial Sequence
  Description of Artificial Sequence: Synthetic peptide
PGDG

SEQ ID NO: 180 - protein, 7 aa, Artificial Sequence
  Description of Artificial Sequence: Synthetic peptide
YGDYTID

SEQ ID NO: 181 - protein, 13 aa, Artificial Sequence
  Description of Artificial Sequence: Synthetic peptide
SQSLLYSSNQKNY

SEQ ID NO: 182 - protein, 3 aa, Artificial Sequence
  Description of Artificial Sequence: Synthetic peptide
WAS

SEQ ID NO: 183 - protein, 5 aa, Artificial Sequence
  Description of Artificial Sequence: Synthetic peptide
YYRYH

SEQ ID NO: 184 - protein, 6 aa, Artificial Sequence
  Description of Artificial Sequence: Synthetic peptide
TDHYIN

SEQ ID NO: 185 - protein, 13 aa, Artificial Sequence
  Description of Artificial Sequence: Synthetic peptide
WIGQIYPGDGNTY

SEQ ID NO: 186 - protein, 10 aa, Artificial Sequence
  Description of Artificial Sequence: Synthetic peptide
APNYGDYTID

SEQ ID NO: 187 - protein, 13 aa, Artificial Sequence
  Description of Artificial Sequence: Synthetic peptide
LYSSNQKNYLAWY

SEQ ID NO: 188 - protein, 10 aa, Artificial Sequence
  Description of Artificial Sequence: Synthetic peptide
LLIYWASTRE

SEQ ID NO: 189 - protein, 7 aa, Artificial Sequence
  Description of Artificial Sequence: Synthetic peptide
QQYYRYH

SEQ ID NO: 190 - protein, 10 aa, Artificial Sequence
  Description of Artificial Sequence: Synthetic peptide
GFTFTDHYIN

SEQ ID NO: 191 - protein, 10 aa, Artificial Sequence
  Description of Artificial Sequence: Synthetic peptide
QIYPGDGNTY

SEQ ID NO: 192 - protein, 9 aa, Artificial Sequence
  Description of Artificial Sequence: Synthetic peptide
NYGDYTIDF

SEQ ID NO: 193 - protein, 17 aa, Artificial Sequence
  Description of Artificial Sequence: Synthetic peptide
KSSQSLLYSSNQKNYLA

SEQ ID NO: 194 - protein, 7 aa, Artificial Sequence
  Description of Artificial Sequence: Synthetic peptide
WASTRES

SEQ ID NO: 195 - protein, 8 aa, Artificial Sequence
  Description of Artificial Sequence: Synthetic peptide
QQYYRYHT

SEQ ID NO: 196 - protein, 10 aa, Artificial Sequence
  Description of Artificial Sequence: Synthetic peptide
GFTFTDHYIN

SEQ ID NO: 197 - protein, 17 aa, Artificial Sequence
  Description of Artificial Sequence: Synthetic peptide
QIYPGDGNTYYNQKFKG

SEQ ID NO: 198 - protein, 9 aa, Artificial Sequence
  Description of Artificial Sequence: Synthetic peptide
NMGMYTIDF

SEQ ID NO: 199 - protein, 17 aa, Artificial Sequence
  Description of Artificial Sequence: Synthetic peptide
KSSQSLLYSSNQKNYLA

SEQ ID NO: 200 - protein, 7 aa, Artificial Sequence
  Description of Artificial Sequence: Synthetic peptide
WASTRES

SEQ ID NO: 201 - protein, 8 aa, Artificial Sequence
  Description of Artificial Sequence: Synthetic peptide
QQYYRYHT

SEQ ID NO: 202 - protein, 8 aa, Artificial Sequence
  Description of Artificial Sequence: Synthetic peptide
GFTFTDHY

SEQ ID NO: 203 - protein, 8 aa, Artificial Sequence
  Description of Artificial Sequence: Synthetic peptide
IYPGDGNT

SEQ ID NO: 204 - protein, 11 aa, Artificial Sequence
  Description of Artificial Sequence: Synthetic peptide
APNMGMYTIDF

SEQ ID NO: 205 - protein, 12 aa, Artificial Sequence
  Description of Artificial Sequence: Synthetic peptide
QSLLYSSNQKNY

SEQ ID NO: 206 - protein, 3 aa, Artificial Sequence
  Description of Artificial Sequence: Synthetic peptide
WAS

SEQ ID NO: 207 - protein, 8 aa, Artificial Sequence
  Description of Artificial Sequence: Synthetic peptide
QQYYRYHT

SEQ ID NO: 208 - protein, 5 aa, Artificial Sequence
  Description of Artificial Sequence: Synthetic peptide
DHYIN

SEQ ID NO: 209 - protein, 17 aa, Artificial Sequence
  Description of Artificial Sequence: Synthetic peptide
QIYPGDGNTYYNQKFKG

SEQ ID NO: 210 - protein, 9 aa, Artificial Sequence
  Description of Artificial Sequence: Synthetic peptide
NMGMYTIDF

SEQ ID NO: 211 - protein, 17 aa, Artificial Sequence
  Description of Artificial Sequence: Synthetic peptide
KSSQSLLYSSNQKNYLA

SEQ ID NO: 212 - protein, 7 aa, Artificial Sequence
  Description of Artificial Sequence: Synthetic peptide
WASTRES

SEQ ID NO: 213 - protein, 8 aa, Artificial Sequence
  Description of Artificial Sequence: Synthetic peptide
QQYYRYHT

SEQ ID NO: 214 - protein, 7 aa, Artificial Sequence
  Description of Artificial Sequence: Synthetic peptide
GFTFTDH

SEQ ID NO: 215 - protein, 4 aa, Artificial Sequence
  Description of Artificial Sequence: Synthetic peptide
PGDG

SEQ ID NO: 216 - protein, 7 aa, Artificial Sequence
  Description of Artificial Sequence: Synthetic peptide
MGMYTID

SEQ ID NO: 217 - protein, 13 aa, Artificial Sequence
  Description of Artificial Sequence: Synthetic peptide
SQSLLYSSNQKNY

SEQ ID NO: 218 - protein, 3 aa, Artificial Sequence
  Description of Artificial Sequence: Synthetic peptide
WAS

SEQ ID NO: 219 - protein, 5 aa, Artificial Sequence
  Description of Artificial Sequence: Synthetic peptide
YYRYH

SEQ ID NO: 220 - protein, 6 aa, Artificial Sequence
  Description of Artificial Sequence: Synthetic peptide
TDHYIN

SEQ ID NO: 221 - protein, 13 aa, Artificial Sequence
  Description of Artificial Sequence: Synthetic peptide
WIGQIYPGDGNTY

SEQ ID NO: 222 - protein, 10 aa, Artificial Sequence
  Description of Artificial Sequence: Synthetic peptide
APNMGMYTID

SEQ ID NO: 223 - protein, 13 aa, Artificial Sequence
  Description of Artificial Sequence: Synthetic peptide
LYSSNQKNYLAWY

SEQ ID NO: 224 - protein, 10 aa, Artificial Sequence
  Description of Artificial Sequence: Synthetic peptide
LLIYWASTRE

SEQ ID NO: 225 - protein, 7 aa, Artificial Sequence
  Description of Artificial Sequence: Synthetic peptide
QQYYRYH

SEQ ID NO: 226 - protein, 10 aa, Artificial Sequence
  Description of Artificial Sequence: Synthetic peptide
GFTFTDHYIN

SEQ ID NO: 227 - protein, 10 aa, Artificial Sequence
  Description of Artificial Sequence: Synthetic peptide
QIYPGDGNTY

SEQ ID NO: 228 - protein, 9 aa, Artificial Sequence
  Description of Artificial Sequence: Synthetic peptide
NMGMYTIDF

SEQ ID NO: 229 - protein, 17 aa, Artificial Sequence
  Description of Artificial Sequence: Synthetic peptide
KSSQSLLYSSNQKNYLA

SEQ ID NO: 230 - protein, 7 aa, Artificial Sequence
  Description of Artificial Sequence: Synthetic peptide
WASTRES

SEQ ID NO: 231 - protein, 8 aa, Artificial Sequence
  Description of Artificial Sequence: Synthetic peptide
QQYYRYHT

SEQ ID NO: 232 - protein, 10 aa, Artificial Sequence
  Description of Artificial Sequence: Synthetic peptide
GFTFTDHYIN

SEQ ID NO: 233 - protein, 17 aa, Artificial Sequence
  Description of Artificial Sequence: Synthetic peptide
QIYPGDGNTYYNQKFKG

SEQ ID NO: 234 - protein, 9 aa, Artificial Sequence
  Description of Artificial Sequence: Synthetic peptide
NMGMYTLDF

SEQ ID NO: 235 - protein, 17 aa, Artificial Sequence
  Description of Artificial Sequence: Synthetic peptide
KSSQSLLYSSNQKNYLA

SEQ ID NO: 236 - protein, 7 aa, Artificial Sequence
  Description of Artificial Sequence: Synthetic peptide
WASTRES

SEQ ID NO: 237 - protein, 8 aa, Artificial Sequence
  Description of Artificial Sequence: Synthetic peptide
QQYYRYHT

SEQ ID NO: 238 - protein, 8 aa, Artificial Sequence
  Description of Artificial Sequence: Synthetic peptide
GFTFTDHY

SEQ ID NO: 239 - protein, 8 aa, Artificial Sequence
  Description of Artificial Sequence: Synthetic peptide
IYPGDGNT

SEQ ID NO: 240 - protein, 11 aa, Artificial Sequence
  Description of Artificial Sequence: Synthetic peptide
APNMGMYTLDF

SEQ ID NO: 241 - protein, 12 aa, Artificial Sequence
  Description of Artificial Sequence: Synthetic peptide
QSLLYSSNQKNY

SEQ ID NO: 242 - protein, 3 aa, Artificial Sequence
  Description of Artificial Sequence: Synthetic peptide
WAS

SEQ ID NO: 243 - protein, 8 aa, Artificial Sequence
  Description of Artificial Sequence: Synthetic peptide
QQYYRYHT

SEQ ID NO: 244 - protein, 5 aa, Artificial Sequence
  Description of Artificial Sequence: Synthetic peptide
DHYIN

SEQ ID NO: 245 - protein, 17 aa, Artificial Sequence
  Description of Artificial Sequence: Synthetic peptide
QIYPGDGNTYYNQKFKG

SEQ ID NO: 246 - protein, 9 aa, Artificial Sequence
  Description of Artificial Sequence: Synthetic peptide
NMGMYTLDF

SEQ ID NO: 247 - protein, 17 aa, Artificial Sequence
  Description of Artificial Sequence: Synthetic peptide
KSSQSLLYSSNQKNYLA

SEQ ID NO: 248 - protein, 7 aa, Artificial Sequence
  Description of Artificial Sequence: Synthetic peptide
WASTRES

SEQ ID NO: 249 - protein, 8 aa, Artificial Sequence
  Description of Artificial Sequence: Synthetic peptide
QQYYRYHT

SEQ ID NO: 250 - protein, 7 aa, Artificial Sequence
  Description of Artificial Sequence: Synthetic peptide
GFTFTDH

SEQ ID NO: 251 - protein, 4 aa, Artificial Sequence
  Description of Artificial Sequence: Synthetic peptide
PGDG

SEQ ID NO: 252 - protein, 7 aa, Artificial Sequence
  Description of Artificial Sequence: Synthetic peptide
MGMYTLD

SEQ ID NO: 253 - protein, 13 aa, Artificial Sequence
  Description of Artificial Sequence: Synthetic peptide
SQSLLYSSNQKNY

SEQ ID NO: 254 - protein, 3 aa, Artificial Sequence
  Description of Artificial Sequence: Synthetic peptide
WAS

SEQ ID NO: 255 - protein, 5 aa, Artificial Sequence
  Description of Artificial Sequence: Synthetic peptide
YYRYH

SEQ ID NO: 256 - protein, 6 aa, Artificial Sequence
  Description of Artificial Sequence: Synthetic peptide
TDHYIN

SEQ ID NO: 257 - protein, 13 aa, Artificial Sequence
  Description of Artificial Sequence: Synthetic peptide
WIGQIYPGDGNTY

SEQ ID NO: 258 - protein, 10 aa, Artificial Sequence
  Description of Artificial Sequence: Synthetic peptide
APNMGMYTLD

SEQ ID NO: 259 - protein, 13 aa, Artificial Sequence
  Description of Artificial Sequence: Synthetic peptide
LYSSNQKNYLAWY

SEQ ID NO: 260 - protein, 10 aa, Artificial Sequence
  Description of Artificial Sequence: Synthetic peptide
LLIYWASTRE

SEQ ID NO: 261 - protein, 7 aa, Artificial Sequence
  Description of Artificial Sequence: Synthetic peptide
QQYYRYH

SEQ ID NO: 262 - protein, 10 aa, Artificial Sequence
  Description of Artificial Sequence: Synthetic peptide
GFTFTDHYIN

SEQ ID NO: 263 - protein, 10 aa, Artificial Sequence
  Description of Artificial Sequence: Synthetic peptide
QIYPGDGNTY

SEQ ID NO: 264 - protein, 9 aa, Artificial Sequence
  Description of Artificial Sequence: Synthetic peptide
NMGMYTLDF

SEQ ID NO: 265 - protein, 17 aa, Artificial Sequence
  Description of Artificial Sequence: Synthetic peptide
KSSQSLLYSSNQKNYLA

SEQ ID NO: 266 - protein, 7 aa, Artificial Sequence
  Description of Artificial Sequence: Synthetic peptide
WASTRES

SEQ ID NO: 267 - protein, 8 aa, Artificial Sequence
  Description of Artificial Sequence: Synthetic peptide
QQYYRYHT

SEQ ID NO: 268 - protein, 10 aa, Artificial Sequence
  Description of Artificial Sequence: Synthetic peptide
GFTFTDHYIN

SEQ ID NO: 269 - protein, 17 aa, Artificial Sequence
  Description of Artificial Sequence: Synthetic peptide
QIYPGDGNTYYNQKFKG

SEQ ID NO: 270 - protein, 9 aa, Artificial Sequence
  Description of Artificial Sequence: Synthetic peptide
NYGDYTLDF

SEQ ID NO: 271 - protein, 17 aa, Artificial Sequence
  Description of Artificial Sequence: Synthetic peptide
KSSQSLLYSSNQKNYLA

SEQ ID NO: 272 - protein, 7 aa, Artificial Sequence
  Description of Artificial Sequence: Synthetic peptide
WASTRES

SEQ ID NO: 273 - protein, 8 aa, Artificial Sequence
  Description of Artificial Sequence: Synthetic peptide
QQYYRYHT

SEQ ID NO: 274 - protein, 8 aa, Artificial Sequence
  Description of Artificial Sequence: Synthetic peptide
GFTFTDHY

SEQ ID NO: 275 - protein, 8 aa, Artificial Sequence
  Description of Artificial Sequence: Synthetic peptide
IYPGDGNT

SEQ ID NO: 276 - protein, 11 aa, Artificial Sequence
  Description of Artificial Sequence: Synthetic peptide
APNYGDYTLDF

SEQ ID NO: 277 - protein, 12 aa, Artificial Sequence
  Description of Artificial Sequence: Synthetic peptide
QSLLYSSNQKNY

SEQ ID NO: 278 - protein, 3 aa, Artificial Sequence
  Description of Artificial Sequence: Synthetic peptide
WAS

SEQ ID NO: 279 - protein, 8 aa, Artificial Sequence
  Description of Artificial Sequence: Synthetic peptide
QQYYRYHT

SEQ ID NO: 280 - protein, 5 aa, Artificial Sequence
  Description of Artificial Sequence: Synthetic peptide
DHYIN

SEQ ID NO: 281 - protein, 17 aa, Artificial Sequence
  Description of Artificial Sequence: Synthetic peptide
QIYPGDGNTYYNQKFKG

SEQ ID NO: 282 - protein, 9 aa, Artificial Sequence
  Description of Artificial Sequence: Synthetic peptide
NYGDYTLDF

SEQ ID NO: 283 - protein, 17 aa, Artificial Sequence
  Description of Artificial Sequence: Synthetic peptide
KSSQSLLYSSNQKNYLA

SEQ ID NO: 284 - protein, 7 aa, Artificial Sequence
  Description of Artificial Sequence: Synthetic peptide
WASTRES

SEQ ID NO: 285 - protein, 8 aa, Artificial Sequence
  Description of Artificial Sequence: Synthetic peptide
QQYYRYHT

SEQ ID NO: 286 - protein, 7 aa, Artificial Sequence
  Description of Artificial Sequence: Synthetic peptide
GFTFTDH

SEQ ID NO: 287 - protein, 4 aa, Artificial Sequence
  Description of Artificial Sequence: Synthetic peptide
PGDG

SEQ ID NO: 288 - protein, 7 aa, Artificial Sequence
  Description of Artificial Sequence: Synthetic peptide
YGDYTLD

SEQ ID NO: 289 - protein, 13 aa, Artificial Sequence
  Description of Artificial Sequence: Synthetic peptide
SQSLLYSSNQKNY

SEQ ID NO: 290 - protein, 3 aa, Artificial Sequence
  Description of Artificial Sequence: Synthetic peptide
WAS

SEQ ID NO: 291 - protein, 5 aa, Artificial Sequence
  Description of Artificial Sequence: Synthetic peptide
YYRYH

SEQ ID NO: 292 - protein, 6 aa, Artificial Sequence
  Description of Artificial Sequence: Synthetic peptide
TDHYIN

SEQ ID NO: 293 - protein, 13 aa, Artificial Sequence
  Description of Artificial Sequence: Synthetic peptide
WIGQIYPGDGNTY

SEQ ID NO: 294 - protein, 10 aa, Artificial Sequence
  Description of Artificial Sequence: Synthetic peptide
APNYGDYTLD

SEQ ID NO: 295 - protein, 13 aa, Artificial Sequence
  Description of Artificial Sequence: Synthetic peptide
LYSSNQKNYLAWY

SEQ ID NO: 296 - protein, 10 aa, Artificial Sequence
  Description of Artificial Sequence: Synthetic peptide
LLIYWASTRE

SEQ ID NO: 297 - protein, 7 aa, Artificial Sequence
  Description of Artificial Sequence: Synthetic peptide
QQYYRYH

SEQ ID NO: 298 - protein, 10 aa, Artificial Sequence
  Description of Artificial Sequence: Synthetic peptide
GFTFTDHYIN

SEQ ID NO: 299 - protein, 10 aa, Artificial Sequence
  Description of Artificial Sequence: Synthetic peptide
QIYPGDGNTY

SEQ ID NO: 300 - protein, 9 aa, Artificial Sequence
  Description of Artificial Sequence: Synthetic peptide
NYGDYTLDF

SEQ ID NO: 301 - protein, 17 aa, Artificial Sequence
  Description of Artificial Sequence: Synthetic peptide
KSSQSLLYSSNQKNYLA

SEQ ID NO: 302 - protein, 7 aa, Artificial Sequence
  Description of Artificial Sequence: Synthetic peptide
WASTRES

SEQ ID NO: 303 - protein, 8 aa, Artificial Sequence
  Description of Artificial Sequence: Synthetic peptide
QQYYRYHT

SEQ ID NO: 304 - protein, 10 aa, Artificial Sequence
  Description of Artificial Sequence: Synthetic peptide
GFTFTDHYIN

SEQ ID NO: 305 - protein, 17 aa, Artificial Sequence
  Description of Artificial Sequence: Synthetic peptide
QIYPGSGNTYYNQKFKG

SEQ ID NO: 306 - protein, 9 aa, Artificial Sequence
  Description of Artificial Sequence: Synthetic peptide
NYGDYTIDF

SEQ ID NO: 307 - protein, 17 aa, Artificial Sequence
  Description of Artificial Sequence: Synthetic peptide
KSSQSVLYSSNNKNYLA

SEQ ID NO: 308 - protein, 7 aa, Artificial Sequence
  Description of Artificial Sequence: Synthetic peptide
WASTRES

SEQ ID NO: 309 - protein, 8 aa, Artificial Sequence
  Description of Artificial Sequence: Synthetic peptide
QQYYRYHT

SEQ ID NO: 310 - protein, 8 aa, Artificial Sequence
  Description of Artificial Sequence: Synthetic peptide
GFTFTDHY

SEQ ID NO: 311 - protein, 8 aa, Artificial Sequence
  Description of Artificial Sequence: Synthetic peptide
IYPGSGNT

SEQ ID NO: 312 - protein, 11 aa, Artificial Sequence
  Description of Artificial Sequence: Synthetic peptide
ARNYGDYTIDF

SEQ ID NO: 313 - protein, 12 aa, Artificial Sequence
  Description of Artificial Sequence: Synthetic peptide
QSVLYSSNNKNY

SEQ ID NO: 314 - protein, 3 aa, Artificial Sequence
  Description of Artificial Sequence: Synthetic peptide
WAS

SEQ ID NO: 315 - protein, 8 aa, Artificial Sequence
  Description of Artificial Sequence: Synthetic peptide
QQYYRYHT

SEQ ID NO: 316 - protein, 5 aa, Artificial Sequence
  Description of Artificial Sequence: Synthetic peptide
DHYIN

SEQ ID NO: 317 - protein, 17 aa, Artificial Sequence
  Description of Artificial Sequence: Synthetic peptide
QIYPGSGNTYYNQKFKG

SEQ ID NO: 318 - protein, 9 aa, Artificial Sequence
  Description of Artificial Sequence: Synthetic peptide
NYGDYTIDF

SEQ ID NO: 319 - protein, 17 aa, Artificial Sequence
  Description of Artificial Sequence: Synthetic peptide
KSSQSVLYSSNNKNYLA

SEQ ID NO: 320 - protein, 7 aa, Artificial Sequence
  Description of Artificial Sequence: Synthetic peptide
WASTRES

SEQ ID NO: 321 - protein, 8 aa, Artificial Sequence
  Description of Artificial Sequence: Synthetic peptide
QQYYRYHT

SEQ ID NO: 322 - protein, 7 aa, Artificial Sequence
  Description of Artificial Sequence: Synthetic peptide
GFTFTDH

SEQ ID NO: 323 - protein, 4 aa, Artificial Sequence
  Description of Artificial Sequence: Synthetic peptide
PGSG

SEQ ID NO: 324 - protein, 7 aa, Artificial Sequence
  Description of Artificial Sequence: Synthetic peptide
YGDYTID

SEQ ID NO: 325 - protein, 13 aa, Artificial Sequence
  Description of Artificial Sequence: Synthetic peptide
SQSVLYSSNNKNY

SEQ ID NO: 326 - protein, 3 aa, Artificial Sequence
  Description of Artificial Sequence: Synthetic peptide
WAS

SEQ ID NO: 327 - protein, 5 aa, Artificial Sequence
  Description of Artificial Sequence: Synthetic peptide
YYRYH

SEQ ID NO: 328 - protein, 6 aa, Artificial Sequence
  Description of Artificial Sequence: Synthetic peptide
TDHYIN

SEQ ID NO: 329 - protein, 13 aa, Artificial Sequence
  Description of Artificial Sequence: Synthetic peptide
WMGQIYPGSGNTY

SEQ ID NO: 330 - protein, 10 aa, Artificial Sequence
  Description of Artificial Sequence: Synthetic peptide
ARNYGDYTID

SEQ ID NO: 331 - protein, 13 aa, Artificial Sequence
  Description of Artificial Sequence: Synthetic peptide
LYSSNNKNYLAWY

SEQ ID NO: 332 - protein, 10 aa, Artificial Sequence
  Description of Artificial Sequence: Synthetic peptide
LLIYWASTRE

SEQ ID NO: 333 - protein, 7 aa, Artificial Sequence
  Description of Artificial Sequence: Synthetic peptide
QQYYRYH

SEQ ID NO: 334 - protein, 10 aa, Artificial Sequence
  Description of Artificial Sequence: Synthetic peptide
GFTFTDHYIN

SEQ ID NO: 335 - protein, 10 aa, Artificial Sequence
  Description of Artificial Sequence: Synthetic peptide
QIYPGSGNTY

SEQ ID NO: 336 - protein, 9 aa, Artificial Sequence
  Description of Artificial Sequence: Synthetic peptide
NYGDYTIDF

SEQ ID NO: 337 - protein, 17 aa, Artificial Sequence
  Description of Artificial Sequence: Synthetic peptide
KSSQSVLYSSNNKNYLA

SEQ ID NO: 338 - protein, 7 aa, Artificial Sequence
  Description of Artificial Sequence: Synthetic peptide
WASTRES

SEQ ID NO: 339 - protein, 8 aa, Artificial Sequence
  Description of Artificial Sequence: Synthetic peptide
QQYYRYHT

SEQ ID NO: 340 - protein, 10 aa, Artificial Sequence
  Description of Artificial Sequence: Synthetic peptide
GFTFTDHYIN

SEQ ID NO: 341 - protein, 17 aa, Artificial Sequence
  Description of Artificial Sequence: Synthetic peptide
QIYPGSGNTYYNQKFKG

SEQ ID NO: 342 - protein, 9 aa, Artificial Sequence
  Description of Artificial Sequence: Synthetic peptide
NYGDYTIDF

SEQ ID NO: 343 - protein, 17 aa, Artificial Sequence
  Description of Artificial Sequence: Synthetic peptide
KSSQSVLYSSNNKNYLA

SEQ ID NO: 344 - protein, 7 aa, Artificial Sequence
  Description of Artificial Sequence: Synthetic peptide
WASTRES

SEQ ID NO: 345 - protein, 8 aa, Artificial Sequence
  Description of Artificial Sequence: Synthetic peptide
QQYYRYHT

SEQ ID NO: 346 - protein, 8 aa, Artificial Sequence
  Description of Artificial Sequence: Synthetic peptide
GFTFTDHY

SEQ ID NO: 347 - protein, 8 aa, Artificial Sequence
  Description of Artificial Sequence: Synthetic peptide
IYPGSGNT

SEQ ID NO: 348 - protein, 11 aa, Artificial Sequence
  Description of Artificial Sequence: Synthetic peptide
ARNYGDYTIDF

SEQ ID NO: 349 - protein, 12 aa, Artificial Sequence
  Description of Artificial Sequence: Synthetic peptide
QSVLYSSNNKNY

SEQ ID NO: 350 - protein, 3 aa, Artificial Sequence
  Description of Artificial Sequence: Synthetic peptide
WAS

SEQ ID NO: 351 - protein, 8 aa, Artificial Sequence
  Description of Artificial Sequence: Synthetic peptide
QQYYRYHT

SEQ ID NO: 352 - protein, 5 aa, Artificial Sequence
  Description of Artificial Sequence: Synthetic peptide
DHYIN

SEQ ID NO: 353 - protein, 17 aa, Artificial Sequence
  Description of Artificial Sequence: Synthetic peptide
QIYPGSGNTYYNQKFKG

SEQ ID NO: 354 - protein, 9 aa, Artificial Sequence
  Description of Artificial Sequence: Synthetic peptide
NYGDYTIDF

SEQ ID NO: 355 - protein, 17 aa, Artificial Sequence
  Description of Artificial Sequence: Synthetic peptide
KSSQSVLYSSNNKNYLA

SEQ ID NO: 356 - protein, 7 aa, Artificial Sequence
  Description of Artificial Sequence: Synthetic peptide
WASTRES

SEQ ID NO: 357 - protein, 8 aa, Artificial Sequence
  Description of Artificial Sequence: Synthetic peptide
QQYYRYHT

SEQ ID NO: 358 - protein, 7 aa, Artificial Sequence
  Description of Artificial Sequence: Synthetic peptide
GFTFTDH

SEQ ID NO: 359 - protein, 4 aa, Artificial Sequence
  Description of Artificial Sequence: Synthetic peptide
PGSG

SEQ ID NO: 360 - protein, 7 aa, Artificial Sequence
  Description of Artificial Sequence: Synthetic peptide
YGDYTID

SEQ ID NO: 361 - protein, 13 aa, Artificial Sequence
  Description of Artificial Sequence: Synthetic peptide
SQSVLYSSNNKNY

SEQ ID NO: 362 - protein, 3 aa, Artificial Sequence
  Description of Artificial Sequence: Synthetic peptide
WAS

SEQ ID NO: 363 - protein, 5 aa, Artificial Sequence
  Description of Artificial Sequence: Synthetic peptide
YYRYH

SEQ ID NO: 364 - protein, 6 aa, Artificial Sequence
  Description of Artificial Sequence: Synthetic peptide
TDHYIN

SEQ ID NO: 365 - protein, 13 aa, Artificial Sequence
  Description of Artificial Sequence: Synthetic peptide
WMGQIYPGSGNTY

SEQ ID NO: 366 - protein, 10 aa, Artificial Sequence
  Description of Artificial Sequence: Synthetic peptide
ARNYGDYTID

SEQ ID NO: 367 - protein, 13 aa, Artificial Sequence
  Description of Artificial Sequence: Synthetic peptide
LYSSNNKNYLAWY

SEQ ID NO: 368 - protein, 10 aa, Artificial Sequence
  Description of Artificial Sequence: Synthetic peptide
LLIYWASTRE

SEQ ID NO: 369 - protein, 7 aa, Artificial Sequence
  Description of Artificial Sequence: Synthetic peptide
QQYYRYH

SEQ ID NO: 370 - protein, 10 aa, Artificial Sequence
  Description of Artificial Sequence: Synthetic peptide
GFTFTDHYIN

SEQ ID NO: 371 - protein, 10 aa, Artificial Sequence
  Description of Artificial Sequence: Synthetic peptide
QIYPGSGNTY

SEQ ID NO: 372 - protein, 9 aa, Artificial Sequence
  Description of Artificial Sequence: Synthetic peptide
NYGDYTIDF

SEQ ID NO: 373 - protein, 17 aa, Artificial Sequence
  Description of Artificial Sequence: Synthetic peptide
KSSQSVLYSSNNKNYLA

SEQ ID NO: 374 - protein, 7 aa, Artificial Sequence
  Description of Artificial Sequence: Synthetic peptide
WASTRES

SEQ ID NO: 375 - protein, 8 aa, Artificial Sequence
  Description of Artificial Sequence: Synthetic peptide
QQYYRYHT

SEQ ID NO: 376 - protein, 10 aa, Artificial Sequence
  Description of Artificial Sequence: Synthetic peptide
GFTFSSNYIS

SEQ ID NO: 377 - protein, 17 aa, Artificial Sequence
  Description of Artificial Sequence: Synthetic peptide
WIHGGTGGIGYNQKFTG

SEQ ID NO: 378 - protein, 9 aa, Artificial Sequence
  Description of Artificial Sequence: Synthetic peptide
RGYGAWFAY

SEQ ID NO: 379 - protein, 11 aa, Artificial Sequence
  Description of Artificial Sequence: Synthetic peptide
RASENIHNYLA

SEQ ID NO: 380 - protein, 7 aa, Artificial Sequence
  Description of Artificial Sequence: Synthetic peptide
NAKTLAD

SEQ ID NO: 381 - protein, 9 aa, Artificial Sequence
  Description of Artificial Sequence: Synthetic peptide
QHFWSYPLT

SEQ ID NO: 382 - protein, 8 aa, Artificial Sequence
  Description of Artificial Sequence: Synthetic peptide
GFTFSSNY

SEQ ID NO: 383 - protein, 8 aa, Artificial Sequence
  Description of Artificial Sequence: Synthetic peptide
IHGGTGGI

SEQ ID NO: 384 - protein, 11 aa, Artificial Sequence
  Description of Artificial Sequence: Synthetic peptide
ARRGYGAWFAY

SEQ ID NO: 385 - protein, 6 aa, Artificial Sequence
  Description of Artificial Sequence: Synthetic peptide
ENIHNY

SEQ ID NO: 386 - protein, 3 aa, Artificial Sequence
  Description of Artificial Sequence: Synthetic peptide
NAK

SEQ ID NO: 387 - protein, 9 aa, Artificial Sequence
  Description of Artificial Sequence: Synthetic peptide
QHFWSYPLT

SEQ ID NO: 388 - protein, 5 aa, Artificial Sequence
  Description of Artificial Sequence: Synthetic peptide
SNYIS

SEQ ID NO: 389 - protein, 17 aa, Artificial Sequence
  Description of Artificial Sequence: Synthetic peptide
WIHGGTGGIGYNQKFTG

SEQ ID NO: 390 - protein, 9 aa, Artificial Sequence
  Description of Artificial Sequence: Synthetic peptide
RGYGAWFAY

SEQ ID NO: 391 - protein, 11 aa, Artificial Sequence
  Description of Artificial Sequence: Synthetic peptide
RASENIHNYLA

SEQ ID NO: 392 - protein, 7 aa, Artificial Sequence
  Description of Artificial Sequence: Synthetic peptide
NAKTLAD

SEQ ID NO: 393 - protein, 9 aa, Artificial Sequence
  Description of Artificial Sequence: Synthetic peptide
QHFWSYPLT

SEQ ID NO: 394 - protein, 7 aa, Artificial Sequence
  Description of Artificial Sequence: Synthetic peptide
GFTFSSN

SEQ ID NO: 395 - protein, 4 aa, Artificial Sequence
  Description of Artificial Sequence: Synthetic peptide
GGTG

SEQ ID NO: 396 - protein, 7 aa, Artificial Sequence
  Description of Artificial Sequence: Synthetic peptide
GYGAWFA

SEQ ID NO: 397 - protein, 7 aa, Artificial Sequence
  Description of Artificial Sequence: Synthetic peptide
SENIHNY

SEQ ID NO: 398 - protein, 3 aa, Artificial Sequence
  Description of Artificial Sequence: Synthetic peptide
NAK

SEQ ID NO: 399 - protein, 6 aa, Artificial Sequence
  Description of Artificial Sequence: Synthetic peptide
FWSYPL

SEQ ID NO: 400 - protein, 6 aa, Artificial Sequence
  Description of Artificial Sequence: Synthetic peptide
SSNYIS

SEQ ID NO: 401 - protein, 13 aa, Artificial Sequence
  Description of Artificial Sequence: Synthetic peptide
WIAWIHGGTGGIG

SEQ ID NO: 402 - protein, 10 aa, Artificial Sequence
  Description of Artificial Sequence: Synthetic peptide
ARRGYGAWFA

SEQ ID NO: 403 - protein, 7 aa, Artificial Sequence
  Description of Artificial Sequence: Synthetic peptide
HNYLAWY

SEQ ID NO: 404 - protein, 10 aa, Artificial Sequence
  Description of Artificial Sequence: Synthetic peptide
LLVYNAKTLA

SEQ ID NO: 405 - protein, 8 aa, Artificial Sequence
  Description of Artificial Sequence: Synthetic peptide
QHFWSYPL

SEQ ID NO: 406 - protein, 10 aa, Artificial Sequence
  Description of Artificial Sequence: Synthetic peptide
GFTFSSNYIS

SEQ ID NO: 407 - protein, 10 aa, Artificial Sequence
  Description of Artificial Sequence: Synthetic peptide
WIHGGTGGIG

SEQ ID NO: 408 - protein, 9 aa, Artificial Sequence
  Description of Artificial Sequence: Synthetic peptide
RGYGAWFAY

SEQ ID NO: 409 - protein, 11 aa, Artificial Sequence
  Description of Artificial Sequence: Synthetic peptide
RASENIHNYLA

SEQ ID NO: 410 - protein, 7 aa, Artificial Sequence
  Description of Artificial Sequence: Synthetic peptide
NAKTLAD

SEQ ID NO: 411 - protein, 9 aa, Artificial Sequence
  Description of Artificial Sequence: Synthetic peptide
QHFWSYPLT

SEQ ID NO: 412 - protein, 10 aa, Artificial Sequence
  Description of Artificial Sequence: Synthetic peptide
GFTFSNYDMN

SEQ ID NO: 413 - protein, 17 aa, Artificial Sequence
  Description of Artificial Sequence: Synthetic peptide
SISSSSSYIYYADSVKG

SEQ ID NO: 414 - protein, 15 aa, Artificial Sequence
  Description of Artificial Sequence: Synthetic peptide
DVGVTTDYYYYGMDV

SEQ ID NO: 415 - protein, 12 aa, Artificial Sequence
  Description of Artificial Sequence: Synthetic peptide
RASQSVASSYLA

SEQ ID NO: 416 - protein, 7 aa, Artificial Sequence
  Description of Artificial Sequence: Synthetic peptide
GASSRAT

SEQ ID NO: 417 - protein, 10 aa, Artificial Sequence
  Description of Artificial Sequence: Synthetic peptide
QQYGSSPPYT

SEQ ID NO: 418 - protein, 8 aa, Artificial Sequence
  Description of Artificial Sequence: Synthetic peptide
GFTFSNYD

SEQ ID NO: 419 - protein, 8 aa, Artificial Sequence
  Description of Artificial Sequence: Synthetic peptide
ISSSSSYI

SEQ ID NO: 420 - protein, 17 aa, Artificial Sequence
  Description of Artificial Sequence: Synthetic peptide
ARDVGVTTDYYYYGMDV

SEQ ID NO: 421 - protein, 7 aa, Artificial Sequence
  Description of Artificial Sequence: Synthetic peptide
QSVASSY

SEQ ID NO: 422 - protein, 3 aa, Artificial Sequence
  Description of Artificial Sequence: Synthetic peptide
GAS

SEQ ID NO: 423 - protein, 10 aa, Artificial Sequence
  Description of Artificial Sequence: Synthetic peptide
QQYGSSPPYT

SEQ ID NO: 424 - protein, 5 aa, Artificial Sequence
  Description of Artificial Sequence: Synthetic peptide
NYDMN

SEQ ID NO: 425 - protein, 17 aa, Artificial Sequence
  Description of Artificial Sequence: Synthetic peptide
SISSSSSYIYYADSVKG

SEQ ID NO: 426 - protein, 15 aa, Artificial Sequence
  Description of Artificial Sequence: Synthetic peptide
DVGVTTDYYYYGMDV

SEQ ID NO: 427 - protein, 12 aa, Artificial Sequence
  Description of Artificial Sequence: Synthetic peptide
RASQSVASSYLA

SEQ ID NO: 428 - protein, 7 aa, Artificial Sequence
  Description of Artificial Sequence: Synthetic peptide
GASSRAT

SEQ ID NO: 429 - protein, 10 aa, Artificial Sequence
  Description of Artificial Sequence: Synthetic peptide
QQYGSSPPYT

SEQ ID NO: 430 - protein, 7 aa, Artificial Sequence
  Description of Artificial Sequence: Synthetic peptide
GFTFSNY

SEQ ID NO: 431 - protein, 4 aa, Artificial Sequence
  Description of Artificial Sequence: Synthetic peptide
SSSS

SEQ ID NO: 432 - protein, 13 aa, Artificial Sequence
  Description of Artificial Sequence: Synthetic peptide
VGVTTDYYYYGMD

SEQ ID NO: 433 - protein, 8 aa, Artificial Sequence
  Description of Artificial Sequence: Synthetic peptide
SQSVASSY

SEQ ID NO: 434 - protein, 3 aa, Artificial Sequence
  Description of Artificial Sequence: Synthetic peptide
GAS

SEQ ID NO: 435 - protein, 7 aa, Artificial Sequence
  Description of Artificial Sequence: Synthetic peptide
YGSSPPY

SEQ ID NO: 436 - protein, 6 aa, Artificial Sequence
  Description of Artificial Sequence: Synthetic peptide
SNYDMN

SEQ ID NO: 437 - protein, 13 aa, Artificial Sequence
  Description of Artificial Sequence: Synthetic peptide
WVSSISSSSSYIY

SEQ ID NO: 438 - protein, 16 aa, Artificial Sequence
  Description of Artificial Sequence: Synthetic peptide
ARDVGVTTDYYYYGMD

SEQ ID NO: 439 - protein, 8 aa, Artificial Sequence
  Description of Artificial Sequence: Synthetic peptide
ASSYLAWY

SEQ ID NO: 440 - protein, 10 aa, Artificial Sequence
  Description of Artificial Sequence: Synthetic peptide
LLIYGASSRA

SEQ ID NO: 441 - protein, 9 aa, Artificial Sequence
  Description of Artificial Sequence: Synthetic peptide
QQYGSSPPY

SEQ ID NO: 442 - protein, 10 aa, Artificial Sequence
  Description of Artificial Sequence: Synthetic peptide
GFTFSNYDMN

SEQ ID NO: 443 - protein, 10 aa, Artificial Sequence
  Description of Artificial Sequence: Synthetic peptide
SISSSSSYIY

SEQ ID NO: 444 - protein, 15 aa, Artificial Sequence
  Description of Artificial Sequence: Synthetic peptide
DVGVTTDYYYYGMDV

SEQ ID NO: 445 - protein, 12 aa, Artificial Sequence
  Description of Artificial Sequence: Synthetic peptide
RASQSVASSYLA

SEQ ID NO: 446 - protein, 7 aa, Artificial Sequence
  Description of Artificial Sequence: Synthetic peptide
GASSRAT

SEQ ID NO: 447 - protein, 10 aa, Artificial Sequence
  Description of Artificial Sequence: Synthetic peptide
QQYGSSPPYT

SEQ ID NO: 448 - protein, 10 aa, Artificial Sequence
  Description of Artificial Sequence: Synthetic peptide
GDTFNNYAIS

SEQ ID NO: 449 - protein, 17 aa, Artificial Sequence
  Description of Artificial Sequence: Synthetic peptide
GIIPFFGTPDYAQKFQG

SEQ ID NO: 450 - protein, 15 aa, Artificial Sequence
  Description of Artificial Sequence: Synthetic peptide
PGSGSPDYYYYDMDV

SEQ ID NO: 451 - protein, 16 aa, Artificial Sequence
  Description of Artificial Sequence: Synthetic peptide
RSSQSLVHSDGNTYLS

SEQ ID NO: 452 - protein, 7 aa, Artificial Sequence
  Description of Artificial Sequence: Synthetic peptide
KISNRFS

SEQ ID NO: 453 - protein, 9 aa, Artificial Sequence
  Description of Artificial Sequence: Synthetic peptide
MQATQFPLT

SEQ ID NO: 454 - protein, 8 aa, Artificial Sequence
  Description of Artificial Sequence: Synthetic peptide
GDTFNNYA

SEQ ID NO: 455 - protein, 8 aa, Artificial Sequence
  Description of Artificial Sequence: Synthetic peptide
IIPFFGTP

SEQ ID NO: 456 - protein, 17 aa, Artificial Sequence
  Description of Artificial Sequence: Synthetic peptide
ARPGSGSPDYYYYDMDV

SEQ ID NO: 457 - protein, 11 aa, Artificial Sequence
  Description of Artificial Sequence: Synthetic peptide
QSLVHSDGNTY

SEQ ID NO: 458 - protein, 3 aa, Artificial Sequence
  Description of Artificial Sequence: Synthetic peptide
KIS

SEQ ID NO: 459 - protein, 9 aa, Artificial Sequence
  Description of Artificial Sequence: Synthetic peptide
MQATQFPLT

SEQ ID NO: 460 - protein, 5 aa, Artificial Sequence
  Description of Artificial Sequence: Synthetic peptide
NYAIS

SEQ ID NO: 461 - protein, 17 aa, Artificial Sequence
  Description of Artificial Sequence: Synthetic peptide
GIIPFFGTPDYAQKFQG

SEQ ID NO: 462 - protein, 15 aa, Artificial Sequence
  Description of Artificial Sequence: Synthetic peptide
PGSGSPDYYYYDMDV

SEQ ID NO: 463 - protein, 16 aa, Artificial Sequence
  Description of Artificial Sequence: Synthetic peptide
RSSQSLVHSDGNTYLS

SEQ ID NO: 464 - protein, 7 aa, Artificial Sequence
  Description of Artificial Sequence: Synthetic peptide
KISNRFS

SEQ ID NO: 465 - protein, 9 aa, Artificial Sequence
  Description of Artificial Sequence: Synthetic peptide
MQATQFPLT

SEQ ID NO: 466 - protein, 7 aa, Artificial Sequence
  Description of Artificial Sequence: Synthetic peptide
GDTFNNY

SEQ ID NO: 467 - protein, 4 aa, Artificial Sequence
  Description of Artificial Sequence: Synthetic peptide
PFFG

SEQ ID NO: 468 - protein, 13 aa, Artificial Sequence
  Description of Artificial Sequence: Synthetic peptide
GSGSPDYYYYDMD

SEQ ID NO: 469 - protein, 12 aa, Artificial Sequence
  Description of Artificial Sequence: Synthetic peptide
SQSLVHSDGNTY

SEQ ID NO: 470 - protein, 3 aa, Artificial Sequence
  Description of Artificial Sequence: Synthetic peptide
KIS

SEQ ID NO: 471 - protein, 6 aa, Artificial Sequence
  Description of Artificial Sequence: Synthetic peptide
ATQFPL

SEQ ID NO: 472 - protein, 6 aa, Artificial Sequence
  Description of Artificial Sequence: Synthetic peptide
NNYAIS

SEQ ID NO: 473 - protein, 13 aa, Artificial Sequence
  Description of Artificial Sequence: Synthetic peptide
WMGGIIPFFGTPD

SEQ ID NO: 474 - protein, 16 aa, Artificial Sequence
  Description of Artificial Sequence: Synthetic peptide
ARPGSGSPDYYYYDMD

SEQ ID NO: 475 - protein, 12 aa, Artificial Sequence
  Description of Artificial Sequence: Synthetic peptide
VHSDGNTYLSWL

SEQ ID NO: 476 - protein, 10 aa, Artificial Sequence
  Description of Artificial Sequence: Synthetic peptide
LLIYKISNRF

SEQ ID NO: 477 - protein, 8 aa, Artificial Sequence
  Description of Artificial Sequence: Synthetic peptide
MQATQFPL

SEQ ID NO: 478 - protein, 10 aa, Artificial Sequence
  Description of Artificial Sequence: Synthetic peptide
GDTFNNYAIS

SEQ ID NO: 479 - protein, 10 aa, Artificial Sequence
  Description of Artificial Sequence: Synthetic peptide
GIIPFFGTPD

SEQ ID NO: 480 - protein, 15 aa, Artificial Sequence
  Description of Artificial Sequence: Synthetic peptide
PGSGSPDYYYYDMDV

SEQ ID NO: 481 - protein, 16 aa, Artificial Sequence
  Description of Artificial Sequence: Synthetic peptide
RSSQSLVHSDGNTYLS

SEQ ID NO: 482 - protein, 7 aa, Artificial Sequence
  Description of Artificial Sequence: Synthetic peptide
KISNRFS

SEQ ID NO: 483 - protein, 9 aa, Artificial Sequence
  Description of Artificial Sequence: Synthetic peptide
MQATQFPLT

SEQ ID NO: 484 - protein, 10 aa, Artificial Sequence
  Description of Artificial Sequence: Synthetic peptide
GGTFSSYAIS

SEQ ID NO: 485 - protein, 17 aa, Artificial Sequence
  Description of Artificial Sequence: Synthetic peptide
GNIPIFNTANYAQKFQD

SEQ ID NO: 486 - protein, 11 aa, Artificial Sequence
  Description of Artificial Sequence: Synthetic peptide
EGTGYSYGLDY

SEQ ID NO: 487 - protein, 16 aa, Artificial Sequence
  Description of Artificial Sequence: Synthetic peptide
RSSQSLIHSDGNTYLS

SEQ ID NO: 488 - protein, 7 aa, Artificial Sequence
  Description of Artificial Sequence: Synthetic peptide
KISNRFS

SEQ ID NO: 489 - protein, 9 aa, Artificial Sequence
  Description of Artificial Sequence: Synthetic peptide
MQAKQFPIT

SEQ ID NO: 490 - protein, 8 aa, Artificial Sequence
  Description of Artificial Sequence: Synthetic peptide
GGTFSSYA

SEQ ID NO: 491 - protein, 8 aa, Artificial Sequence
  Description of Artificial Sequence: Synthetic peptide
NIPIFNTA

SEQ ID NO: 492 - protein, 13 aa, Artificial Sequence
  Description of Artificial Sequence: Synthetic peptide
VREGTGYSYGLDY

SEQ ID NO: 493 - protein, 11 aa, Artificial Sequence
  Description of Artificial Sequence: Synthetic peptide
QSLIHSDGNTY

SEQ ID NO: 494 - protein, 3 aa, Artificial Sequence
  Description of Artificial Sequence: Synthetic peptide
KIS

SEQ ID NO: 495 - protein, 9 aa, Artificial Sequence
  Description of Artificial Sequence: Synthetic peptide
MQAKQFPIT

SEQ ID NO: 496 - protein, 5 aa, Artificial Sequence
  Description of Artificial Sequence: Synthetic peptide
SYAIS

SEQ ID NO: 497 - protein, 17 aa, Artificial Sequence
  Description of Artificial Sequence: Synthetic peptide
GNIPIFNTANYAQKFQD

SEQ ID NO: 498 - protein, 11 aa, Artificial Sequence
  Description of Artificial Sequence: Synthetic peptide
EGTGYSYGLDY

SEQ ID NO: 499 - protein, 16 aa, Artificial Sequence
  Description of Artificial Sequence: Synthetic peptide
RSSQSLIHSDGNTYLS

SEQ ID NO: 500 - protein, 7 aa, Artificial Sequence
  Description of Artificial Sequence: Synthetic peptide
KISNRFS

SEQ ID NO: 501 - protein, 9 aa, Artificial Sequence
  Description of Artificial Sequence: Synthetic peptide
MQAKQFPIT

SEQ ID NO: 502 - protein, 7 aa, Artificial Sequence
  Description of Artificial Sequence: Synthetic peptide
GGTFSSY

SEQ ID NO: 503 - protein, 4 aa, Artificial Sequence
  Description of Artificial Sequence: Synthetic peptide
PIFN

SEQ ID NO: 504 - protein, 9 aa, Artificial Sequence
  Description of Artificial Sequence: Synthetic peptide
GTGYSYGLD

SEQ ID NO: 505 - protein, 12 aa, Artificial Sequence
  Description of Artificial Sequence: Synthetic peptide
SQSLIHSDGNTY

SEQ ID NO: 506 - protein, 3 aa, Artificial Sequence
  Description of Artificial Sequence: Synthetic peptide
KIS

SEQ ID NO: 507 - protein, 6 aa, Artificial Sequence
  Description of Artificial Sequence: Synthetic peptide
AKQFPI

SEQ ID NO: 508 - protein, 6 aa, Artificial Sequence
  Description of Artificial Sequence: Synthetic peptide
SSYAIS

SEQ ID NO: 509 - protein, 13 aa, Artificial Sequence
  Description of Artificial Sequence: Synthetic peptide
WMGGNIPIFNTAN

SEQ ID NO: 510 - protein, 12 aa, Artificial Sequence
  Description of Artificial Sequence: Synthetic peptide
VREGTGYSYGLD

SEQ ID NO: 511 - protein, 12 aa, Artificial Sequence
  Description of Artificial Sequence: Synthetic peptide
IHSDGNTYLSWL

SEQ ID NO: 512 - protein, 10 aa, Artificial Sequence
  Description of Artificial Sequence: Synthetic peptide
LLIYKISNRF

SEQ ID NO: 513 - protein, 8 aa, Artificial Sequence
  Description of Artificial Sequence: Synthetic peptide
MQAKQFPI

SEQ ID NO: 514 - protein, 10 aa, Artificial Sequence
  Description of Artificial Sequence: Synthetic peptide
GGTFSSYAIS

SEQ ID NO: 515 - protein, 10 aa, Artificial Sequence
  Description of Artificial Sequence: Synthetic peptide
GNIPIFNTAN

SEQ ID NO: 516 - protein, 11 aa, Artificial Sequence
  Description of Artificial Sequence: Synthetic peptide
EGTGYSYGLDY

SEQ ID NO: 517 - protein, 16 aa, Artificial Sequence
  Description of Artificial Sequence: Synthetic peptide
RSSQSLIHSDGNTYLS

SEQ ID NO: 518 - protein, 7 aa, Artificial Sequence
  Description of Artificial Sequence: Synthetic peptide
KISNRFS

SEQ ID NO: 519 - protein, 9 aa, Artificial Sequence
  Description of Artificial Sequence: Synthetic peptide
MQAKQFPIT

SEQ ID NO: 520 - protein, 12 aa, Artificial Sequence
  Description of Artificial Sequence: Synthetic peptide
GGSISSGGSYWS

SEQ ID NO: 521 - protein, 16 aa, Artificial Sequence
  Description of Artificial Sequence: Synthetic peptide
YIYNSGSTYYNPSLKS

SEQ ID NO: 522 - protein, 10 aa, Artificial Sequence
  Description of Artificial Sequence: Synthetic peptide
DSNYEWFFDL

SEQ ID NO: 523 - protein, 11 aa, Artificial Sequence
  Description of Artificial Sequence: Synthetic peptide
RASQSVSSYLA

SEQ ID NO: 524 - protein, 7 aa, Artificial Sequence
  Description of Artificial Sequence: Synthetic peptide
DASNRAT

SEQ ID NO: 525 - protein, 9 aa, Artificial Sequence
  Description of Artificial Sequence: Synthetic peptide
QQRSNWPLT

SEQ ID NO: 526 - protein, 10 aa, Artificial Sequence
  Description of Artificial Sequence: Synthetic peptide
GGSISSGGSY

SEQ ID NO: 527 - protein, 7 aa, Artificial Sequence
  Description of Artificial Sequence: Synthetic peptide
IYNSGST

SEQ ID NO: 528 - protein, 12 aa, Artificial Sequence
  Description of Artificial Sequence: Synthetic peptide
ARDSNYEWFFDL

SEQ ID NO: 529 - protein, 6 aa, Artificial Sequence
  Description of Artificial Sequence: Synthetic peptide
QSVSSY

SEQ ID NO: 530 - protein, 3 aa, Artificial Sequence
  Description of Artificial Sequence: Synthetic peptide
DAS

SEQ ID NO: 531 - protein, 9 aa, Artificial Sequence
  Description of Artificial Sequence: Synthetic peptide
QQRSNWPLT

SEQ ID NO: 532 - protein, 7 aa, Artificial Sequence
  Description of Artificial Sequence: Synthetic peptide
SGGSYWS

SEQ ID NO: 533 - protein, 16 aa, Artificial Sequence
  Description of Artificial Sequence: Synthetic peptide
YIYNSGSTYYNPSLKS

SEQ ID NO: 534 - protein, 10 aa, Artificial Sequence
  Description of Artificial Sequence: Synthetic peptide
DSNYEWFFDL

SEQ ID NO: 535 - protein, 11 aa, Artificial Sequence
  Description of Artificial Sequence: Synthetic peptide
RASQSVSSYLA

SEQ ID NO: 536 - protein, 7 aa, Artificial Sequence
  Description of Artificial Sequence: Synthetic peptide
DASNRAT

SEQ ID NO: 537 - protein, 9 aa, Artificial Sequence
  Description of Artificial Sequence: Synthetic peptide
QQRSNWPLT

SEQ ID NO: 538 - protein, 9 aa, Artificial Sequence
  Description of Artificial Sequence: Synthetic peptide
GGSISSGGS

SEQ ID NO: 539 - protein, 3 aa, Artificial Sequence
  Description of Artificial Sequence: Synthetic peptide
NSG

SEQ ID NO: 540 - protein, 8 aa, Artificial Sequence
  Description of Artificial Sequence: Synthetic peptide
SNYEWFFD

SEQ ID NO: 541 - protein, 7 aa, Artificial Sequence
  Description of Artificial Sequence: Synthetic peptide
SQSVSSY

SEQ ID NO: 542 - protein, 3 aa, Artificial Sequence
  Description of Artificial Sequence: Synthetic peptide
DAS

SEQ ID NO: 543 - protein, 6 aa, Artificial Sequence
  Description of Artificial Sequence: Synthetic peptide
RSNWPL

SEQ ID NO: 544 - protein, 8 aa, Artificial Sequence
  Description of Artificial Sequence: Synthetic peptide
SSGGSYWS

SEQ ID NO: 545 - protein, 12 aa, Artificial Sequence
  Description of Artificial Sequence: Synthetic peptide
WIGYIYNSGSTY

SEQ ID NO: 546 - protein, 11 aa, Artificial Sequence
  Description of Artificial Sequence: Synthetic peptide
ARDSNYEWFFD

SEQ ID NO: 547 - protein, 7 aa, Artificial Sequence
  Description of Artificial Sequence: Synthetic peptide
SSYLAWY

SEQ ID NO: 548 - protein, 10 aa, Artificial Sequence
  Description of Artificial Sequence: Synthetic peptide
LLIYDASNRA

SEQ ID NO: 549 - protein, 8 aa, Artificial Sequence
  Description of Artificial Sequence: Synthetic peptide
QQRSNWPL

SEQ ID NO: 550 - protein, 12 aa, Artificial Sequence
  Description of Artificial Sequence: Synthetic peptide
GGSISSGGSYWS

SEQ ID NO: 551 - protein, 9 aa, Artificial Sequence
  Description of Artificial Sequence: Synthetic peptide
YIYNSGSTY

SEQ ID NO: 552 - protein, 10 aa, Artificial Sequence
  Description of Artificial Sequence: Synthetic peptide
DSNYEWFFDL

SEQ ID NO: 553 - protein, 11 aa, Artificial Sequence
  Description of Artificial Sequence: Synthetic peptide
RASQSVSSYLA

SEQ ID NO: 554 - protein, 7 aa, Artificial Sequence
  Description of Artificial Sequence: Synthetic peptide
DASNRAT

SEQ ID NO: 555 - protein, 9 aa, Artificial Sequence
  Description of Artificial Sequence: Synthetic peptide
QQRSNWPLT

SEQ ID NO: 556 - protein, 10 aa, Artificial Sequence
  Description of Artificial Sequence: Synthetic peptide
GYSFTSYWIS

SEQ ID NO: 557 - protein, 17 aa, Artificial Sequence
  Description of Artificial Sequence: Synthetic peptide
IIDPSDSDTRYSPSFQG

SEQ ID NO: 558 - protein, 9 aa, Artificial Sequence
  Description of Artificial Sequence: Synthetic peptide
GDGSTDLDY

SEQ ID NO: 559 - protein, 12 aa, Artificial Sequence
  Description of Artificial Sequence: Synthetic peptide
RASQSVSSSYLA

SEQ ID NO: 560 - protein, 7 aa, Artificial Sequence
  Description of Artificial Sequence: Synthetic peptide
GASSRAT

SEQ ID NO: 561 - protein, 9 aa, Artificial Sequence
  Description of Artificial Sequence: Synthetic peptide
QQDYGFPWT

SEQ ID NO: 562 - protein, 8 aa, Artificial Sequence
  Description of Artificial Sequence: Synthetic peptide
GYSFTSYW

SEQ ID NO: 563 - protein, 8 aa, Artificial Sequence
  Description of Artificial Sequence: Synthetic peptide
IDPSDSDT

SEQ ID NO: 564 - protein, 11 aa, Artificial Sequence
  Description of Artificial Sequence: Synthetic peptide
ARGDGSTDLDY

SEQ ID NO: 565 - protein, 7 aa, Artificial Sequence
  Description of Artificial Sequence: Synthetic peptide
QSVSSSY

SEQ ID NO: 566 - protein, 3 aa, Artificial Sequence
  Description of Artificial Sequence: Synthetic peptide
GAS

SEQ ID NO: 567 - protein, 9 aa, Artificial Sequence
  Description of Artificial Sequence: Synthetic peptide
QQDYGFPWT

SEQ ID NO: 568 - protein, 5 aa, Artificial Sequence
  Description of Artificial Sequence: Synthetic peptide
SYWIS

SEQ ID NO: 569 - protein, 17 aa, Artificial Sequence
  Description of Artificial Sequence: Synthetic peptide
IIDPSDSDTRYSPSFQG

SEQ ID NO: 570 - protein, 9 aa, Artificial Sequence
  Description of Artificial Sequence: Synthetic peptide
GDGSTDLDY

SEQ ID NO: 571 - protein, 12 aa, Artificial Sequence
  Description of Artificial Sequence: Synthetic peptide
RASQSVSSSYLA

SEQ ID NO: 572 - protein, 7 aa, Artificial Sequence
  Description of Artificial Sequence: Synthetic peptide
GASSRAT

SEQ ID NO: 573 - protein, 9 aa, Artificial Sequence
  Description of Artificial Sequence: Synthetic peptide
QQDYGFPWT

SEQ ID NO: 574 - protein, 7 aa, Artificial Sequence
  Description of Artificial Sequence: Synthetic peptide
GYSFTSY

SEQ ID NO: 575 - protein, 4 aa, Artificial Sequence
  Description of Artificial Sequence: Synthetic peptide
PSDS

SEQ ID NO: 576 - protein, 7 aa, Artificial Sequence
  Description of Artificial Sequence: Synthetic peptide
DGSTDLD

SEQ ID NO: 577 - protein, 8 aa, Artificial Sequence
  Description of Artificial Sequence: Synthetic peptide
SQSVSSSY

SEQ ID NO: 578 - protein, 3 aa, Artificial Sequence
  Description of Artificial Sequence: Synthetic peptide
GAS

SEQ ID NO: 579 - protein, 6 aa, Artificial Sequence
  Description of Artificial Sequence: Synthetic peptide
DYGFPW

SEQ ID NO: 580 - protein, 6 aa, Artificial Sequence
  Description of Artificial Sequence: Synthetic peptide
TSYWIS

SEQ ID NO: 581 - protein, 13 aa, Artificial Sequence
  Description of Artificial Sequence: Synthetic peptide
WMGIIDPSDSDTR

SEQ ID NO: 582 - protein, 10 aa, Artificial Sequence
  Description of Artificial Sequence: Synthetic peptide
ARGDGSTDLD

SEQ ID NO: 583 - protein, 8 aa, Artificial Sequence
  Description of Artificial Sequence: Synthetic peptide
SSSYLAWY

SEQ ID NO: 584 - protein, 10 aa, Artificial Sequence
  Description of Artificial Sequence: Synthetic peptide
LLIYGASSRA

SEQ ID NO: 585 - protein, 8 aa, Artificial Sequence
  Description of Artificial Sequence: Synthetic peptide
QQDYGFPW

SEQ ID NO: 586 - protein, 10 aa, Artificial Sequence
  Description of Artificial Sequence: Synthetic peptide
GYSFTSYWIS

SEQ ID NO: 587 - protein, 10 aa, Artificial Sequence
  Description of Artificial Sequence: Synthetic peptide
IIDPSDSDTR

SEQ ID NO: 588 - protein, 9 aa, Artificial Sequence
  Description of Artificial Sequence: Synthetic peptide
GDGSTDLDY

SEQ ID NO: 589 - protein, 12 aa, Artificial Sequence
  Description of Artificial Sequence: Synthetic peptide
RASQSVSSSYLA

SEQ ID NO: 590 - protein, 7 aa, Artificial Sequence
  Description of Artificial Sequence: Synthetic peptide
GASSRAT

SEQ ID NO: 591 - protein, 9 aa, Artificial Sequence
  Description of Artificial Sequence: Synthetic peptide
QQDYGFPWT

SEQ ID NO: 592 - protein, 10 aa, Artificial Sequence
  Description of Artificial Sequence: Synthetic peptide
GFTFDDYAMH

SEQ ID NO: 593 - protein, 17 aa, Artificial Sequence
  Description of Artificial Sequence: Synthetic peptide
GIGWSGGSIVYADSVKG

SEQ ID NO: 594 - protein, 10 aa, Artificial Sequence
  Description of Artificial Sequence: Synthetic peptide
DSPYGDFFDY

SEQ ID NO: 595 - protein, 17 aa, Artificial Sequence
  Description of Artificial Sequence: Synthetic peptide
KSSQTVFYSSNNKNYLA

SEQ ID NO: 596 - protein, 7 aa, Artificial Sequence
  Description of Artificial Sequence: Synthetic peptide
WASTRKS

SEQ ID NO: 597 - protein, 9 aa, Artificial Sequence
  Description of Artificial Sequence: Synthetic peptide
QHYYSTPYT

SEQ ID NO: 598 - protein, 8 aa, Artificial Sequence
  Description of Artificial Sequence: Synthetic peptide
GFTFDDYA

SEQ ID NO: 599 - protein, 8 aa, Artificial Sequence
  Description of Artificial Sequence: Synthetic peptide
IGWSGGSI

SEQ ID NO: 600 - protein, 12 aa, Artificial Sequence
  Description of Artificial Sequence: Synthetic peptide
AKDSPYGDFFDY

SEQ ID NO: 601 - protein, 12 aa, Artificial Sequence
  Description of Artificial Sequence: Synthetic peptide
QTVFYSSNNKNY

SEQ ID NO: 602 - protein, 3 aa, Artificial Sequence
  Description of Artificial Sequence: Synthetic peptide
WAS

SEQ ID NO: 603 - protein, 9 aa, Artificial Sequence
  Description of Artificial Sequence: Synthetic peptide
QHYYSTPYT

SEQ ID NO: 604 - protein, 5 aa, Artificial Sequence
  Description of Artificial Sequence: Synthetic peptide
DYAMH

SEQ ID NO: 605 - protein, 17 aa, Artificial Sequence
  Description of Artificial Sequence: Synthetic peptide
GIGWSGGSIVYADSVKG

SEQ ID NO: 606 - protein, 10 aa, Artificial Sequence
  Description of Artificial Sequence: Synthetic peptide
DSPYGDFFDY

SEQ ID NO: 607 - protein, 17 aa, Artificial Sequence
  Description of Artificial Sequence: Synthetic peptide
KSSQTVFYSSNNKNYLA

SEQ ID NO: 608 - protein, 7 aa, Artificial Sequence
  Description of Artificial Sequence: Synthetic peptide
WASTRKS

SEQ ID NO: 609 - protein, 9 aa, Artificial Sequence
  Description of Artificial Sequence: Synthetic peptide
QHYYSTPYT

SEQ ID NO: 610 - protein, 7 aa, Artificial Sequence
  Description of Artificial Sequence: Synthetic peptide
GFTFDDY

SEQ ID NO: 611 - protein, 4 aa, Artificial Sequence
  Description of Artificial Sequence: Synthetic peptide
WSGG

SEQ ID NO: 612 - protein, 8 aa, Artificial Sequence
  Description of Artificial Sequence: Synthetic peptide
SPYGDFFD

SEQ ID NO: 613 - protein, 13 aa, Artificial Sequence
  Description of Artificial Sequence: Synthetic peptide
SQTVFYSSNNKNY

SEQ ID NO: 614 - protein, 3 aa, Artificial Sequence
  Description of Artificial Sequence: Synthetic peptide
WAS

SEQ ID NO: 615 - protein, 6 aa, Artificial Sequence
  Description of Artificial Sequence: Synthetic peptide
YYSTPY

SEQ ID NO: 616 - protein, 6 aa, Artificial Sequence
  Description of Artificial Sequence: Synthetic peptide
DDYAMH

SEQ ID NO: 617 - protein, 13 aa, Artificial Sequence
  Description of Artificial Sequence: Synthetic peptide
WVSGIGWSGGSIV

SEQ ID NO: 618 - protein, 11 aa, Artificial Sequence
  Description of Artificial Sequence: Synthetic peptide
AKDSPYGDFFD

SEQ ID NO: 619 - protein, 13 aa, Artificial Sequence
  Description of Artificial Sequence: Synthetic peptide
FYSSNNKNYLAWY

SEQ ID NO: 620 - protein, 10 aa, Artificial Sequence
  Description of Artificial Sequence: Synthetic peptide
LLISWASTRK

SEQ ID NO: 621 - protein, 8 aa, Artificial Sequence
  Description of Artificial Sequence: Synthetic peptide
QHYYSTPY

SEQ ID NO: 622 - protein, 10 aa, Artificial Sequence
  Description of Artificial Sequence: Synthetic peptide
GFTFDDYAMH

SEQ ID NO: 623 - protein, 10 aa, Artificial Sequence
  Description of Artificial Sequence: Synthetic peptide
GIGWSGGSIV

SEQ ID NO: 624 - protein, 10 aa, Artificial Sequence
  Description of Artificial Sequence: Synthetic peptide
DSPYGDFFDY

SEQ ID NO: 625 - protein, 17 aa, Artificial Sequence
  Description of Artificial Sequence: Synthetic peptide
KSSQTVFYSSNNKNYLA

SEQ ID NO: 626 - protein, 7 aa, Artificial Sequence
  Description of Artificial Sequence: Synthetic peptide
WASTRKS

SEQ ID NO: 627 - protein, 9 aa, Artificial Sequence
  Description of Artificial Sequence: Synthetic peptide
QHYYSTPYT

SEQ ID NO: 628 - protein, 10 aa, Artificial Sequence
  Description of Artificial Sequence: Synthetic peptide
GYTFTGYVMH

SEQ ID NO: 629 - protein, 17 aa, Artificial Sequence
  Description of Artificial Sequence: Synthetic peptide
FINPYNDDIQSNERFRG

SEQ ID NO: 630 - protein, 15 aa, Artificial Sequence
  Description of Artificial Sequence: Synthetic peptide
GAGYNFDGAYRFFDF

SEQ ID NO: 631 - protein, 16 aa, Artificial Sequence
  Description of Artificial Sequence: Synthetic peptide
RSSQRLVHSNGNTYLH

SEQ ID NO: 632 - protein, 7 aa, Artificial Sequence
  Description of Artificial Sequence: Synthetic peptide
RVSNRFP

SEQ ID NO: 633 - protein, 9 aa, Artificial Sequence
  Description of Artificial Sequence: Synthetic peptide
SQSTHVPYT

SEQ ID NO: 634 - protein, 8 aa, Artificial Sequence
  Description of Artificial Sequence: Synthetic peptide
GYTFTGYV

SEQ ID NO: 635 - protein, 8 aa, Artificial Sequence
  Description of Artificial Sequence: Synthetic peptide
INPYNDDI

SEQ ID NO: 636 - protein, 17 aa, Artificial Sequence
  Description of Artificial Sequence: Synthetic peptide
ARGAGYNFDGAYRFFDF

SEQ ID NO: 637 - protein, 11 aa, Artificial Sequence
  Description of Artificial Sequence: Synthetic peptide
QRLVHSNGNTY

SEQ ID NO: 638 - protein, 3 aa, Artificial Sequence
  Description of Artificial Sequence: Synthetic peptide
RVS

SEQ ID NO: 639 - protein, 9 aa, Artificial Sequence
  Description of Artificial Sequence: Synthetic peptide
SQSTHVPYT

SEQ ID NO: 640 - protein, 5 aa, Artificial Sequence
  Description of Artificial Sequence: Synthetic peptide
GYVMH

SEQ ID NO: 641 - protein, 17 aa, Artificial Sequence
  Description of Artificial Sequence: Synthetic peptide
FINPYNDDIQSNERFRG

SEQ ID NO: 642 - protein, 15 aa, Artificial Sequence
  Description of Artificial Sequence: Synthetic peptide
GAGYNFDGAYRFFDF

SEQ ID NO: 643 - protein, 16 aa, Artificial Sequence
  Description of Artificial Sequence: Synthetic peptide
RSSQRLVHSNGNTYLH

SEQ ID NO: 644 - protein, 7 aa, Artificial Sequence
  Description of Artificial Sequence: Synthetic peptide
RVSNRFP

SEQ ID NO: 645 - protein, 9 aa, Artificial Sequence
  Description of Artificial Sequence: Synthetic peptide
SQSTHVPYT

SEQ ID NO: 646 - protein, 7 aa, Artificial Sequence
  Description of Artificial Sequence: Synthetic peptide
GYTFTGY

SEQ ID NO: 647 - protein, 4 aa, Artificial Sequence
  Description of Artificial Sequence: Synthetic peptide
PYND

SEQ ID NO: 648 - protein, 13 aa, Artificial Sequence
  Description of Artificial Sequence: Synthetic peptide
AGYNFDGAYRFFD

SEQ ID NO: 649 - protein, 12 aa, Artificial Sequence
  Description of Artificial Sequence: Synthetic peptide
SQRLVHSNGNTY

SEQ ID NO: 650 - protein, 3 aa, Artificial Sequence
  Description of Artificial Sequence: Synthetic peptide
RVS

SEQ ID NO: 651 - protein, 6 aa, Artificial Sequence
  Description of Artificial Sequence: Synthetic peptide
STHVPY

SEQ ID NO: 652 - protein, 6 aa, Artificial Sequence
  Description of Artificial Sequence: Synthetic peptide
TGYVMH

SEQ ID NO: 653 - protein, 13 aa, Artificial Sequence
  Description of Artificial Sequence: Synthetic peptide
WIGFINPYNDDIQ

SEQ ID NO: 654 - protein, 16 aa, Artificial Sequence
  Description of Artificial Sequence: Synthetic peptide
ARGAGYNFDGAYRFFD

SEQ ID NO: 655 - protein, 12 aa, Artificial Sequence
  Description of Artificial Sequence: Synthetic peptide
VHSNGNTYLHWY

SEQ ID NO: 656 - protein, 10 aa, Artificial Sequence
  Description of Artificial Sequence: Synthetic peptide
LLIYRVSNRF

SEQ ID NO: 657 - protein, 8 aa, Artificial Sequence
  Description of Artificial Sequence: Synthetic peptide
SQSTHVPY

SEQ ID NO: 658 - protein, 10 aa, Artificial Sequence
  Description of Artificial Sequence: Synthetic peptide
GYTFTGYVMH

SEQ ID NO: 659 - protein, 10 aa, Artificial Sequence
  Description of Artificial Sequence: Synthetic peptide
FINPYNDDIQ

SEQ ID NO: 660 - protein, 15 aa, Artificial Sequence
  Description of Artificial Sequence: Synthetic peptide
GAGYNFDGAYRFFDF

SEQ ID NO: 661 - protein, 16 aa, Artificial Sequence
  Description of Artificial Sequence: Synthetic peptide
RSSQRLVHSNGNTYLH

SEQ ID NO: 662 - protein, 7 aa, Artificial Sequence
  Description of Artificial Sequence: Synthetic peptide
RVSNRFP

SEQ ID NO: 663 - protein, 9 aa, Artificial Sequence
  Description of Artificial Sequence: Synthetic peptide
SQSTHVPYT

SEQ ID NO: 664 - protein, 10 aa, Artificial Sequence
  Description of Artificial Sequence: Synthetic peptide
GFTFSSYAMS

SEQ ID NO: 665 - protein, 17 aa, Artificial Sequence
  Description of Artificial Sequence: Synthetic peptide
AISGSGGSTYYADSVKG

SEQ ID NO: 666 - protein, 14 aa, Artificial Sequence
  Description of Artificial Sequence: Synthetic peptide
DEGYSSGHYYGMDV

SEQ ID NO: 667 - protein, 12 aa, Artificial Sequence
  Description of Artificial Sequence: Synthetic peptide
RASQSISSSFLT

SEQ ID NO: 668 - protein, 7 aa, Artificial Sequence
  Description of Artificial Sequence: Synthetic peptide
GASSRAT

SEQ ID NO: 669 - protein, 10 aa, Artificial Sequence
  Description of Artificial Sequence: Synthetic peptide
QHYGSSPMYT

SEQ ID NO: 670 - protein, 8 aa, Artificial Sequence
  Description of Artificial Sequence: Synthetic peptide
GFTFSSYA

SEQ ID NO: 671 - protein, 8 aa, Artificial Sequence
  Description of Artificial Sequence: Synthetic peptide
ISGSGGST

SEQ ID NO: 672 - protein, 16 aa, Artificial Sequence
  Description of Artificial Sequence: Synthetic peptide
AKDEGYSSGHYYGMDV

SEQ ID NO: 673 - protein, 7 aa, Artificial Sequence
  Description of Artificial Sequence: Synthetic peptide
QSISSSF

SEQ ID NO: 674 - protein, 3 aa, Artificial Sequence
  Description of Artificial Sequence: Synthetic peptide
GAS

SEQ ID NO: 675 - protein, 10 aa, Artificial Sequence
  Description of Artificial Sequence: Synthetic peptide
QHYGSSPMYT

SEQ ID NO: 676 - protein, 5 aa, Artificial Sequence
  Description of Artificial Sequence: Synthetic peptide
SYAMS

SEQ ID NO: 677 - protein, 17 aa, Artificial Sequence
  Description of Artificial Sequence: Synthetic peptide
AISGSGGSTYYADSVKG

SEQ ID NO: 678 - protein, 14 aa, Artificial Sequence
  Description of Artificial Sequence: Synthetic peptide
DEGYSSGHYYGMDV

SEQ ID NO: 679 - protein, 12 aa, Artificial Sequence
  Description of Artificial Sequence: Synthetic peptide
RASQSISSSFLT

SEQ ID NO: 680 - protein, 7 aa, Artificial Sequence
  Description of Artificial Sequence: Synthetic peptide
GASSRAT

SEQ ID NO: 681 - protein, 10 aa, Artificial Sequence
  Description of Artificial Sequence: Synthetic peptide
QHYGSSPMYT

SEQ ID NO: 682 - protein, 7 aa, Artificial Sequence
  Description of Artificial Sequence: Synthetic peptide
GFTFSSY

SEQ ID NO: 683 - protein, 4 aa, Artificial Sequence
  Description of Artificial Sequence: Synthetic peptide
GSGG

SEQ ID NO: 684 - protein, 12 aa, Artificial Sequence
  Description of Artificial Sequence: Synthetic peptide
EGYSSGHYYGMD

SEQ ID NO: 685 - protein, 8 aa, Artificial Sequence
  Description of Artificial Sequence: Synthetic peptide
SQSISSSF

SEQ ID NO: 686 - protein, 3 aa, Artificial Sequence
  Description of Artificial Sequence: Synthetic peptide
GAS

SEQ ID NO: 687 - protein, 7 aa, Artificial Sequence
  Description of Artificial Sequence: Synthetic peptide
YGSSPMY

SEQ ID NO: 688 - protein, 6 aa, Artificial Sequence
  Description of Artificial Sequence: Synthetic peptide
SSYAMS

SEQ ID NO: 689 - protein, 13 aa, Artificial Sequence
  Description of Artificial Sequence: Synthetic peptide
WVSAISGSGGSTY

SEQ ID NO: 690 - protein, 15 aa, Artificial Sequence
  Description of Artificial Sequence: Synthetic peptide
AKDEGYSSGHYYGMD

SEQ ID NO: 691 - protein, 8 aa, Artificial Sequence
  Description of Artificial Sequence: Synthetic peptide
SSSFLTWY

SEQ ID NO: 692 - protein, 10 aa, Artificial Sequence
  Description of Artificial Sequence: Synthetic peptide
LLIYGASSRA

SEQ ID NO: 693 - protein, 9 aa, Artificial Sequence
  Description of Artificial Sequence: Synthetic peptide
QHYGSSPMY

SEQ ID NO: 694 - protein, 10 aa, Artificial Sequence
  Description of Artificial Sequence: Synthetic peptide
GFTFSSYAMS

SEQ ID NO: 695 - protein, 10 aa, Artificial Sequence
  Description of Artificial Sequence: Synthetic peptide
AISGSGGSTY

SEQ ID NO: 696 - protein, 14 aa, Artificial Sequence
  Description of Artificial Sequence: Synthetic peptide
DEGYSSGHYYGMDV

SEQ ID NO: 697 - protein, 12 aa, Artificial Sequence
  Description of Artificial Sequence: Synthetic peptide
RASQSISSSFLT

SEQ ID NO: 698 - protein, 7 aa, Artificial Sequence
  Description of Artificial Sequence: Synthetic peptide
GASSRAT

SEQ ID NO: 699 - protein, 10 aa, Artificial Sequence
  Description of Artificial Sequence: Synthetic peptide
QHYGSSPMYT

SEQ ID NO: 700 - protein, 6 aa, Artificial Sequence
  Description of Artificial Sequence: Synthetic peptide
YPGDGN

SEQ ID NO: 701 - protein, 8 aa, Artificial Sequence
  Description of Artificial Sequence: Synthetic peptide
NYGDYTID

SEQ ID NO: 702 - protein, 6 aa, Artificial Sequence
  Description of Artificial Sequence: Synthetic peptide
YPGDGN

SEQ ID NO: 703 - protein, 8 aa, Artificial Sequence
  Description of Artificial Sequence: Synthetic peptide
NMGMYTID

SEQ ID NO: 704 - protein, 6 aa, Artificial Sequence
  Description of Artificial Sequence: Synthetic peptide
YPGDGN

SEQ ID NO: 705 - protein, 8 aa, Artificial Sequence
  Description of Artificial Sequence: Synthetic peptide
NMGMYTLD

SEQ ID NO: 706 - protein, 6 aa, Artificial Sequence
  Description of Artificial Sequence: Synthetic peptide
YPGDGN

SEQ ID NO: 707 - protein, 8 aa, Artificial Sequence
  Description of Artificial Sequence: Synthetic peptide
NYGDYTLD

SEQ ID NO: 708 - protein, 6 aa, Artificial Sequence
  Description of Artificial Sequence: Synthetic peptide
YPGSGN

SEQ ID NO: 709 - protein, 8 aa, Artificial Sequence
  Description of Artificial Sequence: Synthetic peptide
NYGDYTID

SEQ ID NO: 710 - protein, 6 aa, Artificial Sequence
  Description of Artificial Sequence: Synthetic peptide
YPGSGN

SEQ ID NO: 711 - protein, 8 aa, Artificial Sequence
  Description of Artificial Sequence: Synthetic peptide
NYGDYTID

SEQ ID NO: 712 - protein, 6 aa, Artificial Sequence
  Description of Artificial Sequence: Synthetic peptide
HGGTGG

SEQ ID NO: 713 - protein, 8 aa, Artificial Sequence
  Description of Artificial Sequence: Synthetic peptide
RGYGAWFA

SEQ ID NO: 714 - protein, 6 aa, Artificial Sequence
  Description of Artificial Sequence: Synthetic peptide
SSSSSY

SEQ ID NO: 715 - protein, 14 aa, Artificial Sequence
  Description of Artificial Sequence: Synthetic peptide
DVGVTTDYYYYGMD

SEQ ID NO: 716 - protein, 6 aa, Artificial Sequence
  Description of Artificial Sequence: Synthetic peptide
IPFFGT

SEQ ID NO: 717 - protein, 14 aa, Artificial Sequence
  Description of Artificial Sequence: Synthetic peptide
PGSGSPDYYYYDMD

SEQ ID NO: 718 - protein, 6 aa, Artificial Sequence
  Description of Artificial Sequence: Synthetic peptide
IPIFNT

SEQ ID NO: 719 - protein, 10 aa, Artificial Sequence
  Description of Artificial Sequence: Synthetic peptide
EGTGYSYGLD

SEQ ID NO: 720 - protein, 5 aa, Artificial Sequence
  Description of Artificial Sequence: Synthetic peptide
YNSGS

SEQ ID NO: 721 - protein, 9 aa, Artificial Sequence
  Description of Artificial Sequence: Synthetic peptide
DSNYEWFFD

SEQ ID NO: 722 - protein, 6 aa, Artificial Sequence
  Description of Artificial Sequence: Synthetic peptide
DPSDSD

SEQ ID NO: 723 - protein, 8 aa, Artificial Sequence
  Description of Artificial Sequence: Synthetic peptide
GDGSTDLD

SEQ ID NO: 724-729 - skipped (6 entries)

SEQ ID NO: 730 - protein, 10 aa, Artificial Sequence
  Description of Artificial Sequence: Synthetic peptide
GRTFSRYTMG

SEQ ID NO: 731 - protein, 17 aa, Artificial Sequence
  Description of Artificial Sequence: Synthetic peptide
AISWSGGRTNFAGSVKG

SEQ ID NO: 732 - protein, 13 aa, Artificial Sequence
  Description of Artificial Sequence: Synthetic peptide
DWLPVPGRESYDY

SEQ ID NO: 733 - protein, 10 aa, Artificial Sequence
  Description of Artificial Sequence: Synthetic peptide
GRTFSSYAMG

SEQ ID NO: 734 - protein, 17 aa, Artificial Sequence
  Description of Artificial Sequence: Synthetic peptide
AISWSGGTTYYADSVKG

SEQ ID NO: 735 - protein, 16 aa, Artificial Sequence
  Description of Artificial Sequence: Synthetic peptide
SLDCSGPGCHTAEYDY

SEQ ID NO: 736 - protein, 10 aa, Artificial Sequence
  Description of Artificial Sequence: Synthetic peptide
GRTFSEYAMG

SEQ ID NO: 737 - protein, 17 aa, Artificial Sequence
  Description of Artificial Sequence: Synthetic peptide
AISWTGSKTYYADSVKG

SEQ ID NO: 738 - protein, 16 aa, Artificial Sequence
  Description of Artificial Sequence: Synthetic peptide
SSDCSGPGCHTEEYDY

SEQ ID NO: 739 - protein, 10 aa, Artificial Sequence
  Description of Artificial Sequence: Synthetic peptide
GRTFSSYAMG

SEQ ID NO: 740 - protein, 17 aa, Artificial Sequence
  Description of Artificial Sequence: Synthetic peptide
AVSWSGGSTYYADSVKG

SEQ ID NO: 741 - protein, 16 aa, Artificial Sequence
  Description of Artificial Sequence: Synthetic peptide
SQDCSGPGCYTNEYDS

SEQ ID NO: 742 - protein, 10 aa, Artificial Sequence
  Description of Artificial Sequence: Synthetic peptide
GSIFSNYAMA

SEQ ID NO: 743 - protein, 17 aa, Artificial Sequence
  Description of Artificial Sequence: Synthetic peptide
AVSWSGGRTYYADSVKG

SEQ ID NO: 744 - protein, 16 aa, Artificial Sequence
  Description of Artificial Sequence: Synthetic peptide
SLSCSGPGCSLEEYDY

SEQ ID NO: 745 - protein, 10 aa, Artificial Sequence
  Description of Artificial Sequence: Synthetic peptide
GRPFSNYGMG

SEQ ID NO: 746 - protein, 17 aa, Artificial Sequence
  Description of Artificial Sequence: Synthetic peptide
GITWSGGSTHYADLVKG

SEQ ID NO: 747 - protein, 17 aa, Artificial Sequence
  Description of Artificial Sequence: Synthetic peptide
VFSGAETAYYPSTEYDY

SEQ ID NO: 748 - protein, 10 aa, Artificial Sequence
  Description of Artificial Sequence: Synthetic peptide
VRTFSDYRMG

SEQ ID NO: 749 - protein, 17 aa, Artificial Sequence
  Description of Artificial Sequence: Synthetic peptide
TISWSGGLTYYADSVKG

SEQ ID NO: 750 - protein, 14 aa, Artificial Sequence
  Description of Artificial Sequence: Synthetic peptide
GGGYAGGTYYHPEE

SEQ ID NO: 751 - protein, 122 aa, Artificial Sequence
  Description of Artificial Sequence: Synthetic polypeptide
EVQLVESGGGLVQAGDSLRLSCAASGRTFSRYTMGWFRQAPGKEREFVAAISWSGGRTNFAGSVKGRFTISRDNAKNTVYLQMNSLKPEDTAVYYCAADWLPVPGRESYDYWGQGTQVTVSS

SEQ ID NO: 752 - protein, 125 aa, Artificial Sequence
  Description of Artificial Sequence: Synthetic polypeptide
EVQLVESGGGLVQAGGSLRLSCTASGRTFSSYAMGWFRQAPGKEREFVAAISWSGGTTYYADSVKGRFTISRDNAKNTVSLQMNSLKPEDTAVYFCAASLDCSGPGCHTAEYDYWGQGTQVTVSS

SEQ ID NO: 753 - protein, 125 aa, Artificial Sequence
  Description of Artificial Sequence: Synthetic polypeptide
EVQLVESGGGLVQAGGSLRLSCAATGRTFSEYAMGWFRQAPGKEREFAAAISWIGSKTYYADSVKGRFTISRDNAKNTVYLQMNSLKPEDTAVYYCAASSDCSGPGCHTEEYDYWGQGTQVTVSS

SEQ ID NO: 754 - protein, 125 aa, Artificial Sequence
  Description of Artificial Sequence: Synthetic polypeptide
EVQLVESGGGLVQAGGSLRLSCAASGRTFSSYAMGWFRQAPGKEREFVAAVSWSGGSTYYADSVKGRFTISRDNARNTVYLQMNSLNPEDTAVYYCAASQDCSGPGCYTNEYDSWGQGTQVTVSS

SEQ ID NO: 755 - protein, 125 aa, Artificial Sequence
  Description of Artificial Sequence: Synthetic polypeptide
EVQLVESGGGLVQPGGSLRLSCAASGSIFSNYAMAWFRQAPEKERDFLAAVSWSGGRTYYADSVKGRFTISRDNAKNTVNLQMNSLKPEDTAVYYCAASLSCSGPGCSLEEYDYWGQGTQVTVSS

SEQ ID NO: 756 - protein, 126 aa, Artificial Sequence
  Description of Artificial Sequence: Synthetic polypeptide
EVQLVESGGGLVQAGGSLRLSCAVSGRPFSNYGMGWFRQAPGKEREFVAGITWSGGSTHYADLVKGRFTISRDNAKNTVHLQMNSLKPEDTAVYYCAAVFSGAETAYYPSTEYDYWGQGTQVTVSS

SEQ ID NO: 757 - protein, 123 aa, Artificial Sequence
  Description of Artificial Sequence: Synthetic polypeptide
EVQLVESGGGLVQAGGSLRLSCAVSVRTFSDYRMGWFRQAPGKEREFVSTISWSGGLTYYADSVKGRFTISRDNSKNTLYLQMNSLKPEDTAVYYCAAGGGYAGGTYYHPEEWGQGTQVTVSS

SEQ ID NO: 758 - protein, 11 aa, Artificial Sequence
  Description of Artificial Sequence: Synthetic peptide
  MOD_RES (4)..(4): Tyr or Met
  MOD_RES (6)..(6): Met or Asp
  MOD_RES (9)..(9): Ile or Leu
APNXGXYTXDF

SEQ ID NO: 759-760 - skipped (2 entries)

SEQ ID NO: 761 - protein, 6 aa, Artificial Sequence
  Description of Artificial Sequence: Synthetic peptide
  MOD_RES (2)..(2): Phe, Asp, or Gly
  MOD_RES (5)..(6): Ser or Asn
GXTFXX

SEQ ID NO: 762 - protein, 6 aa, Artificial Sequence
  Description of Artificial Sequence: Synthetic peptide
  MOD_RES (2)..(2): Ala or Ser
  MOD_RES (6)..(6): Val or Leu
RXSQSX

SEQ ID NO: 763 - protein, 242 aa, Artificial Sequence
  Vg9_Cg9
AGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 764 - protein, 229 aa, Artificial Sequence
  Vd2_Cd2
AIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 765 - protein, 98 aa, Mus
  mouse IGHV1-76*01 VH
QVQLKQSGAELVRPGASVKLSCKASGYTFTDYYINWVKQRPGQGLEWIARIYPGSGNTYYNEKFKGKATLTAEKSSSTAYMQLSSLTSEDSAVYFCAR

SEQ ID NO: 766 - protein, 98 aa, Homo sapiens
  human IGHV1-8*01 VH
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 767 - protein, 118 aa, Homo sapiens
  human Graft VH
QVQLVQSGAEVKKPGASVKVSCKASGFTFTDHYINWVRQATGQGLEWMGQIYPGDGNTYYNQKFKGRVTMTRNTSISTAYMELSSLRSEDTAVYYCAPNYGDYTIDFWGQGTSVTVSS

SEQ ID NO: 768 - protein, 112 aa, Artificial Sequence
  Design VH
VQLQSGAEVKPGASVKVSCKASGFTFTDHYINWVRQATGQGLEWMGQIYPGGNTYYNQKFKGRVTMTRTSISTAYMELSSLRSEDTAVYYCANYGDYTIDFWGQGTSVTVSS

SEQ ID NO: 769 - protein, 101 aa, Mus
  mouse IGKV8-30*01 VL
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYP

SEQ ID NO: 770 - protein, 101 aa, Homo sapiens
  human IGKV4-1*01 VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 771 - protein, 112 aa, Homo sapiens; identical to SEQ ID NO: 66
  human Graft VL

SEQ ID NO: 772 - protein, 108 aa, Artificial Sequence
  Design VL
DIVMQSPDSLAVSLGERTCKSSQSVLYSSNNKNYLAWYQQKPGQPPKLLIYWASTRESGVPDRFSGSGSGTDFTLTISSLQAEDVAVYYCQQYYRYHTFGTGTKLEIK

SEQ ID NO: 773 - protein, 20 aa, Homo sapiens
  Residues 49-68 on human Vg9-Vd2 TCR
LVSISYDGTVRKESGIPSGK

SEQ ID NO: 774 - protein, 20 aa, Homo sapiens
  Residues 49 to 68 of human PSMA
LVSISYDGTVRKESGIPSGK

SEQ ID NO: 775 - protein, 124 aa, Artificial Sequence
  PSMA antibody VH (Clone PSMB365-PSMH865)
EVQLLESGGGLVQPGGSLRLSCAASGFTFKSDAMHWVRQAPGKGLEWVSEISGSGGYTNYADSLKSRFTISRDNSKNTLYLQMNSLRAEDTAVYYCARDSYDSSLYVGDYFDYWGQGTLVTVSS

SEQ ID NO: 776 - protein, 107 aa, Artificial Sequence
  PSMA antibody VL (Clone PSMB365-PSML160)
EIVLTQSPATLSLSPGERATLSCRASQSVSSYLAWYQQKPGQAPRLLIYDASYRATGIPARFSGSGSGTDFTLTISSLEPEDFAVYYCQQRRNWPLTFGQGTKVEIK

SEQ ID NO: 777 - protein, 7 aa, Artificial Sequence
  TRGV9 antibody VH-CDR1
  misc_feature (2)..(2): Xaa = Phe and Asp
  misc_feature (5)..(5): Xaa = Thr, Ser or Asn
  misc_feature (6)..(6): Xaa = Asp, Ser or Asn
  misc_feature (7)..(7): Xaa = His, Asn or Tyr
GXTFXXX

SEQ ID NO: 778 - protein, 4 aa, Artificial Sequence
  TRGV9 antibody VH-CDR2
  misc_feature (3)..(3): Xaa = Asp or Ser
PGXG

SEQ ID NO: 779 - protein, 7 aa, Artificial Sequence
  TRGV9 antibody VH-CDR3
  misc_feature (1)..(1): Xaa = Tyr or Met
  misc_feature (3)..(3): Xaa = Asp or Met
  misc_feature (6)..(6): Xaa = Ile or Leu
XGXYTXD

SEQ ID NO: 780 - protein, 11 aa, Artificial Sequence
  TRGV9 antibody VL-CDR1
  misc_feature (4)..(4): Xaa Leu or Val
  misc_feature (10)..(10): Xaa = Gln or Asn
  misc_feature (11)..(11): Xaa = Lyr or Lyr-Ans-Tyr
SQSXLYSSNXX

SEQ ID NO: 781 - protein, 450 aa, Artificial Sequence
  PSMA antibody HC (Clone PSMB365)
EVQLLESGGGLVQPGGSLRLSCAASGFTFKSDAMHWVRQAPGKGLEWVSEISGSGGYTNYADSLKSRFTISRDNSKNTLYLQMNSLRAEDTAVYYCARDSYDSSLYVGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCWVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 782 - protein, 214 aa, Artificial Sequence
  PSMA antibody LC (Clone PSMB365)
EIVLTQSPATLSLSPGERATLSCRASQSVSSYLAWYQQKPGQAPRLLIYDASYRATGIPARFSGSGSGTDFTLTISSLEPEDFAVYYCQQRRNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 783 - protein, 5 aa, Artificial Sequence
  PSMA antibody VH-CDR1 (Clone PSMB365-PSMH865)
SDAMH

SEQ ID NO: 784 - protein, 17 aa, Artificial Sequence
  PSMA antibody VH-CDR2 (Clone PSMB365-PSMH865)
EISGSGGYTNYADSLKS

SEQ ID NO: 785 - protein, 15 aa, Artificial Sequence
  PSMA antibody VH-CDR3 (Clone PSMB365-PSMH865)
DSYDSSLYVGDYFDY

SEQ ID NO: 786 - protein, 11 aa, Artificial Sequence
  PSMA antibody VL-CDR1 (Clone PSMB365-PSML160)
RASQSVSSYLA

SEQ ID NO: 787 - protein, 7 aa, Artificial Sequence
  PSMA antibody VL-CDR2 (Clone PSMB365-PSML160)
DASYRAT

SEQ ID NO: 788 - protein, 9 aa, Artificial Sequence
  PSMA antibody VL-CDR3 (Clone PSMB365-PSML160)
QQRRNWPLT

SEQ ID NO: 789 - protein, 330 aa, Homo sapiens
  Partial Sequence of human VG9
AGHLEQPQISSTKTLSKTARLECVVSGITISATSVYWYRERPGEVIQFLVSISYDGTVRKESGIPSGKFEVDRIPETSTSTLTIHNVEAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 790 - protein, 229 aa, Homo sapiens; identical to SEQ ID NO: 764
  Partial Sequence of human VD2

(19) United States
(12) Patent Application Publication
(10) Pub. No.: US 2021/0284731 A1
(43) Pub. Date: Sep. 16, 2021

(54) METHODS AND MATERIALS FOR MODULATING AN IMMUNE RESPONSE

(71) Applicant: Janssen Biotech, Inc., Horsham, PA (US)

(72) Inventors: Rajkumar Ganesan, Blue Bell, PA (US); Iqbal S. Grewal, Newtown, PA (US); Sanjaya Singh, Blue Bell, PA (US)

(73) Assignee: Janssen Biotech, Inc., Horsham, PA (US)

(21) Appl. No.: 17/202,004

(22) Filed: Mar. 15, 2021

Related U.S. Application Data
(63) Continuation-in-part of application No. PCT/US2020/031749, filed on May 7, 2020.
Application No. PCT/US2020/031749 is a continuation-in-part of application No. PCT/US2021/019766, filed on Feb. 26, 2021.
(60) Provisional application No. 62/988,996, filed on Mar. 13, 2020.
(60) Provisional application No. 62/989,002, filed on Mar. 13, 2020.
(60) Provisional application No. 62/989,006, filed on Mar. 13, 2020.
(60) Provisional application No. 62/989,010, filed on Mar. 13, 2020.
(60) Provisional application No. 62/989,018, filed on Mar. 13, 2020.
(60) Provisional application No. 62/989,024, filed on Mar. 13, 2020.
(60) Provisional application No. 62/989,027, filed on Mar. 13, 2020.
(60) Provisional application No. 62/989,036, filed on Mar. 13, 2020.
(60) Provisional application No. 62/989,042, filed on Mar. 13, 2020.
(60) Provisional application No. 62/989,045, filed on Mar. 13, 2020.
(60) Provisional application No. 62/989,052, filed on Mar. 13, 2020.
(60) Provisional application No. 62/989,057, filed on Mar. 13, 2020.
(60) Provisional application No. 62/989,068, filed on Mar. 13, 2020.
(60) Provisional application No. 62/989,075, filed on Mar. 13, 2020.
(60) Provisional application No. 63/074,655, filed on Sep. 4, 2020.
(60) Provisional application No. 63/074,676, filed on Sep. 4, 2020.
(60) Provisional application No. 63/074,854, filed on Sep. 4, 2020.
(60) Provisional application No. 63/074,700, filed on Sep. 4, 2020.
(60) Provisional application No. 63/074,749, filed on Sep. 4, 2020.
(60) Provisional application No. 63/074,735, filed on Sep. 4, 2020.
(60) Provisional application No. 63/074,839, filed on Sep. 4, 2020.
(60) Provisional application No. 63/074,759, filed on Sep. 4, 2020.
(60) Provisional application No. 63/074,903, filed on Sep. 4, 2020.
(60) Provisional application No. 63/074,893, filed on Sep. 4, 2020.
(60) Provisional application No. 63/074,925, filed on Sep. 4, 2020.
(60) Provisional application No. 63/074,937, filed on Sep. 4, 2020.
(60) Provisional application No. 63/074,962, filed on Sep. 4, 2020.
(60) Provisional application No. 63/074,946, filed on Sep. 4, 2020.
(60) Provisional application No. 63/112,462, filed on Nov. 11, 2020.
(60) Provisional application No. 63/112,475, filed on Nov. 11, 2020.

(51) Int. Cl. C07K 16/28; C07K 16/30; A61P 35/00

(52) U.S. Cl. CPC C07K 16/2809; C07K 16/2878; C07K 16/3069; C07K 16/2866; C07K 2317/73; C07K 2317/565; C07K 2317/31; C07K 2317/24; A61P 35/00

(57) ABSTRACT

Anti-TRGV9 molecules, such as anti-TRGV9 antibodies or antigen binding fragments thereof, are described. Also described are nucleic acids encoding the antibodies, compositions comprising the antibodies, methods of producing the antibodies, and methods of using the antibodies for treating or preventing diseases.

### CROSS-REFERENCE TO RELATED APPLICATIONS

This application claims the benefit of U.S. Ser. No. 62/988,996 filed Mar. 13, 2020; U.S. Ser. No. 62/989,002 filed Mar. 13, 2020; U.S. Ser. No. 62/989,006 filed Mar. 13, 2020; U.S. Ser. No. 62/989,010 filed Mar. 13, 2020; U.S. Ser. No. 62/989,018 filed Mar. 13, 2020; U.S. Ser. No. 62/989,024 filed Mar. 13, 2020; U.S. Ser. No. 62/989,027 filed Mar. 13, 2020; U.S. Ser. No. 62/989,036 filed Mar. 13, 2020; U.S. Ser. No. 62/989,042 filed Mar. 13, 2020; U.S. Ser. No. 62/989,045 filed Mar. 13, 2020; U.S. Ser. No. 62/989,052 filed Mar. 13, 2020; U.S. Ser. No. 62/989,057 filed Mar. 13, 2020; U.S. Ser. No. 62/989,068 filed Mar. 13, 2020; U.S. Ser. No. 62/989,075 filed Mar. 13, 2020; International Serial No. PCT/US20/31749 filed May 7, 2020; U.S. Ser. No. 63/074,655 filed Sep. 4, 2020; U.S. Ser. No. 63/074,676 filed Sep. 4, 2020; U.S. Ser. No. 63/074,854 filed Sep. 4, 2020; U.S. Ser. No. 63/074,700 filed Sep. 4, 2020; U.S. Ser. No. 63/074,749 filed Sep. 4, 2020; U.S. Ser. No. 63/074,735 filed Sep. 4, 2020; U.S. Ser. No. 63/074,839 filed Sep. 4, 2020; U.S. Ser. No. 63/074,759 filed Sep. 4, 2020; U.S. Ser. No. 63/074,903 filed Sep. 4, 2020; U.S. Ser. No. 63/074,893 filed Sep. 4, 2020; U.S. Ser. No. 63/074,925 filed Sep. 4, 2020; U.S. Ser. No. 63/074,937 filed Sep. 4, 2020; U.S. Ser. No. 63/074,962 filed Sep. 4, 2020; U.S. Ser. No. 63/074,946 filed Sep. 4, 2020; U.S. Ser. No. 63/112,462 filed Nov. 11, 2020; U.S. Ser. No. 63/112,475 filed Nov. 11, 2020; and International Serial No. PCT/US21/19766 filed Feb. 26, 2021, each of which is herein incorporated by reference in its entirety.

### FIELD

This invention relates to, among other things, T Cell Receptor (TCR) redirection technologies, such as those targeting T cell Receptor Gamma Variable 9 (TRGV9) molecules, including TRGV9 antibodies, bispecific antibodies, nucleic acids and expression vectors encoding the antibodies, recombinant cells containing the vectors, and compositions comprising the antibodies. Methods of making the antibodies, and methods of using the antibodies to modulate an immune response to cancer cells, are also provided.

### REFERENCE TO SEQUENCE LISTING SUBMITTED ELECTRONICALLY

This application contains a sequence listing, which is submitted electronically via EFS-Web as an ASCII formatted sequence listing with a file “14620-450-999_SEQLISTING” and a creation date of Mar. 14, 2021 and having a size of 440,188 bytes. The sequence listing submitted via EFS-Web is part of the specification and is herein incorporated by reference in its entirety.

### SUMMARY

Provided are T Cell Receptor redirection technologies, including, for example, TRGV9 molecules, such as TRGV9 antibodies and multispecific antibodies, as well as nucleic acids and expression vectors encoding the antibodies, recombinant cells containing the vectors, and compositions comprising the antibodies.

In one aspect, provided herein is an antibody that binds to TRGV9. In some embodiments, the antibody comprises a heavy chain variable region (VH) and a light chain variable region (VL).

In one embodiment, the TRGV9 antibody has a VH and VL amino acid sequence of L7A5_2 (TRGV9_2). In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:34; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8.

In one embodiment, the TRGV9 antibody has a VH and VL amino acid sequence of L7A5_3 (TRGV9_3). In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:35; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8.

In one embodiment, the TRGV9 antibody has a VH and VL amino acid sequence of L7A5_4 (TRGV9_4). In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:36; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8.

In one embodiment, the TRGV9 antibody has a VH and VL amino acid sequence of TRGV9Ab_var17. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66.

In one embodiment, the TRGV9 antibody has a VH and VL amino acid sequence of TRGV9Ab_var29. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68.

In one embodiment, the TRGV9 antibody has a VH and VL amino acid sequence of VG9B420. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:104; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:105.

In one embodiment, the TRGV9 antibody has a VH and VL amino acid sequence of VG9SB10SC1087_P18_D08. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:113; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:114.

In one embodiment, the TRGV9 antibody has a VH and VL amino acid sequence of VG9SB10SC1087_P18_C12. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:123; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:124.

In one embodiment, the TRGV9 antibody has a VH and VL amino acid sequence of VG9SB10SC1087_P19_C03. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134.

In some embodiments, the VH CDR1, VH CDR2, VH CDR3, VL CDR1, VL CDR2, and VL CDR3 amino acid sequences of the TRGV9 antibody are according to the Kabat numbering system. In some embodiments, the VH CDR1, VH CDR2, VH CDR3, VL CDR1, VL CDR2, and VL CDR3 amino acid sequences of the TRGV9 antibody are according to the Chothia numbering system. In some embodiments, the VH CDR1, VH CDR2, VH CDR3, VL CDR1, VL CDR2, and VL CDR3 amino acid sequences of the TRGV9 antibody are according to the AbM numbering system. In some embodiments, the VH CDR1, VH CDR2, VH CDR3, VL CDR1, VL CDR2, and VL CDR3 amino acid sequences of the TRGV9 antibody are according to the Contact numbering system. In some embodiments, the VH CDR1, VH CDR2, VH CDR3, VL CDR1, VL CDR2, and VL CDR3 amino acid sequences of the TRGV9 antibody are according to the IMGT numbering system. In some embodiments, the VH CDR1, VH CDR2, VH CDR3, VL CDR1, VL CDR2, and VL CDR3 amino acid sequences of the TRGV9 antibody are according to the Exemplary numbering system.

In some embodiments, the antibody binds a TRGV9 antigen. In some embodiments, antibody binds a TRGV9 epitope. In some embodiments, the antibody specifically binds to TRGV9. In some embodiments, the VH CDR1, VH CDR2, VH CDR3, VL CDR1, VL CDR2 and VL CDR3 form a binding site for an antigen of the TRGV9. In some embodiments, the VH CDR1, VH CDR2, VH CDR3, VL CDR1, VL CDR2 and VL CDR3 form a binding site for an epitope of the TRGV9. In some embodiments, the TRGV9 is present on the surface of a T cell. In some embodiments, the T cell is a γδ T cell.

In some embodiments, the TRGV9 antibody is chimeric. In some embodiments, the TRGV9 antibody is human. In some embodiments, the TRGV9 antibody is humanized. In certain embodiments, the TRGV9 antibody is an isolated TRGV9 antibody. In some embodiments, the TRGV9 antibody is a TRGV9 antigen binding fragment. In some embodiments, the TRGV9 antigen binding fragment is chimeric. In some embodiments, the TRGV9 antigen binding fragment is human. In some embodiments, a TRGV9 antigen binding fragment is humanized. In certain embodiments, a TRGV9 antigen binding fragment is an isolated TRGV9 antigen binding fragment. In some embodiments, the TRGV9 antibody is an IgG antibody. In some embodiments, the TRGV9 antibody is an IgG1 antibody. In some embodiments, the TRGV9 antibody is an IgG2 antibody. In some embodiments, the TRGV9 antibody is an IgG3 antibody. In some embodiments, the TRGV9 antibody is an IgG4 antibody. In some embodiments, the TRGV9 antibody comprises a kappa light chain. In some embodiments, the TRGV9 antibody comprises a lambda light chain. In some embodiments, the TRGV9 antibody is a monoclonal antibody. In some embodiments, the TRGV9 antibody is multivalent. In some embodiments, the TRGV9 antibody is capable of binding at least three antigens. In some embodiments, the TRGV9 antibody is capable of binding at least four antigens. In some embodiments, the TRGV9 antibody is capable of binding at least five antigens. In some embodiments, the TRGV9 antibody is a multispecific antibody. In some embodiments, the TRGV9 antibody is a bispecific antibody. In some embodiments, the TRGV9 antibody is a trispecific antibody. In some embodiments, the TRGV9 antibody is a quadraspecific antibody.

In one specific embodiment, the TRGV9 antibody is a multispecific TRGV9 antibody. In one specific embodiment, the multispecific TRGV9 antibody is a bispecific TRGV9 antibody.

Thus, in one aspect, provided is a multispecific TRGV9 antibody, comprising a TRGV9 antibody provided herein. In one embodiment, the multispecific TRGV9 antibody comprises: (a) a first binding domain that binds to TRGV9, and (b) a second binding domain that binds to a second target that is not TRGV9.

In one embodiment, the first binding domain that binds to TRGV9 comprises a VH and VL amino acid sequence of L7A5_1 (TRGV9_1). In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:7; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8.

In one embodiment, the first binding domain that binds to TRGV9 comprises a VH and VL amino acid sequence of L7A5_2 (TRGV9_2). In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:34; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8.

In one embodiment, the first binding domain that binds to TRGV9 comprises a VH and VL amino acid sequence of L7A5_3 (TRGV9_3). In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:35; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8.

In one embodiment, the first binding domain that binds to TRGV9 comprises a VH and VL amino acid sequence of L7A5_4 (TRGV9_4). In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:36; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8.

In one embodiment, the first binding domain that binds to TRGV9 comprises a VH and VL amino acid sequence of TRGV9Ab_var17. In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66.

In one embodiment, the first binding domain that binds to TRGV9 comprises a VH and VL amino acid sequence of TRGV9Ab_var29. In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68.

In one embodiment, the first binding domain that binds to TRGV9 comprises a VH and VL amino acid sequence of VG9_B3_RN. In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96.

In one embodiment, the first binding domain that binds to TRGV9 comprises a VH and VL amino acid sequence of VG9B420. In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:104; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:105.

In one embodiment, the first binding domain that binds to TRGV9 comprises a VH and VL amino acid sequence of VG9SB10SC1087_P18_D08. In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:113; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:114.

In one embodiment, the first binding domain that binds to TRGV9 comprises a VH and VL amino acid sequence of VG9SB10SC1087_P18_C12. In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:123; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:124.

In one embodiment, the first binding domain that binds to TRGV9 comprises a VH and VL amino acid sequence of VG9SB10SC1087_P19_C03. In one embodiment, the first binding domain that binds to TRGV9 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134.

In some embodiments, the VH CDR1, VH CDR2, VH CDR3, VL CDR1, VL CDR2, and VL CDR3 amino acid sequences of the first binding domain that binds TRGV9 are according to the Kabat numbering system. In some embodiments, the VH CDR1, VH CDR2, VH CDR3, VL CDR1, VL CDR2, and VL CDR3 amino acid sequences of the first binding domain that binds TRGV9 are according to the Chothia numbering system. In some embodiments, the VH CDR1, VH CDR2, VH CDR3, VL CDR1, VL CDR2, and VL CDR3 amino acid sequences of the first binding domain that binds TRGV9 are according to the AbM numbering system. In some embodiments, the VH CDR1, VH CDR2, VH CDR3, VL CDR1, VL CDR2, and VL CDR3 amino acid sequences of the first binding domain that binds TRGV9 are according to the Contact numbering system. In some embodiments, the VH CDR1, VH CDR2, VH CDR3, VL CDR1, VL CDR2, and VL CDR3 amino acid sequences of the first binding domain that binds TRGV9 are according to the IMGT numbering system. In some embodiments, the VH CDR1, VH CDR2, VH CDR3, VL CDR1, VL CDR2, and VL CDR3 amino acid sequences of the first binding domain that binds TRGV9 are according to the Exemplary numbering system.

In some embodiments, the first binding domain binds a TRGV9 antigen. In some embodiments, the first binding domain binds a TRGV9 epitope. In some embodiments, the first binding domain specifically binds to TRGV9. In some embodiments, the VH CDR1, VH CDR2, VH CDR3, VL CDR1, VL CDR2 and VL CDR3 of the first binding domain form a binding site for an antigen of the TRGV9. In some embodiments, the VH CDR1, VH CDR2, VH CDR3, VL CDR1, VL CDR2 and VL CDR3 of the first binding domain form a binding site for an epitope of the TRGV9. In some embodiments, the TRGV9 is present on the surface of a T cell.

In some embodiments of the multispecific TRGV9 antibodies provided herein, the second target is not a TRGV9 antigen. In some embodiments of the multispecific TRGV9 antibodies provided herein, the second target is not a TRGV9 epitope.

In some embodiments of the multispecific TRGV9 antibodies provided herein, the second target is CD123. In one embodiment, the second binding domain that binds to CD123 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:15; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:16.

In some embodiments of the multispecific TRGV9 antibodies provided herein, the second target is CD33. In one embodiment, the second target is the C2 domain of CD33. the second target is the V domain of CD33. In one embodiment, the second binding domain that binds to CD33 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:43; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:44.

In some embodiments of the multispecific TRGV9 antibodies provided herein, the second target is TRBC1. In one embodiment, the second binding domain that binds to TRBC1 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:55; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:56.

In some embodiments of the multispecific TRGV9 antibodies provided herein, the second target is B cell maturation antigen (BCMA). In one embodiment, the second binding domain binds to BCMA. In one embodiment, the second binding domain that binds to BCMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:143; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:144.

In some embodiments of the multispecific TRGV9 antibodies provided herein, the second target is prostate-specific membrane antigen (PSMA). In one embodiment, the second binding domain binds to PSMA. In one embodiment, the second binding domain that binds to PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:775; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:776.

In some embodiments of the multispecific TRGV9 antibodies provided herein, the second target is expressed by target cell. In some embodiments of the multispecific TRGV9 antibodies provided herein, the second target is on the surface of a target cell. In a specific embodiment, the target cell is an undesired cell.

In one embodiment, the target cell is a cancer cell. In one embodiment, the target cell is a T cell. In one embodiment, the target cell is a B cell. In one embodiment, the target cell is a dendritic cell. In one embodiment, the target cell is a NK cell. In one embodiment, the target cell is a stem cell. In one embodiment, the target cell is a stem cell precursor. In one embodiment, the target cell is a monocyte. In one embodiment, the target cell is a macrophage. In one embodiment, the target cell is a granulocyte. In one embodiment, the target cell is a platelet. In one embodiment, the target cell is an erythrocyte. In one embodiment, the target cell is an endothelial cell. In one embodiment, the target cell is an epithelial cell. In one embodiment, the second target is a pathogen. In one embodiment, the target cell is a cell comprising a pathogen. In one embodiment, the target cell is a blood cell. In one embodiment, the target cell is a myeloid cell.

In some embodiments, the VH CDR1, VH CDR2, VH CDR3, VL CDR1, VL CDR2, and VL CDR3 amino acid sequences of the second binding domain that binds the second target are according to the Kabat numbering system. In some embodiments, the VH CDR1, VH CDR2, VH CDR3, VL CDR1, VL CDR2, and VL CDR3 amino acid sequences of the second binding domain that binds the second target are according to the Chothia numbering system. In some embodiments, the VH CDR1, VH CDR2, VH CDR3, VL CDR1, VL CDR2, and VL CDR3 amino acid sequences of the second binding domain that binds the second target are according to the AbM numbering system. In some embodiments, the VH CDR1, VH CDR2, VH CDR3, VL CDR1, VL CDR2, and VL CDR3 amino acid sequences of the second binding domain that binds the second target are according to the Contact numbering system. In some embodiments, the VH CDR1, VH CDR2, VH CDR3, VL CDR1, VL CDR2, and VL CDR3 amino acid sequences of the second binding domain that binds the second target are according to the IMGT numbering system. In some embodiments, the VH CDR1, VH CDR2, VH CDR3, VL CDR1, VL CDR2, and VL CDR3 amino acid sequences of the second binding domain that binds the second target are according to the Exemplary numbering system.

In some embodiments, the second binding domain binds an antigen of the second target. In some embodiments, In some embodiments, the second binding domain binds an epitope of the second target. In some embodiments, the second binding domain specifically binds to the second target. In some embodiments, the second binding domain specifically binds an antigen of the second target. In some embodiments, In some embodiments, the second binding domain specifically binds an epitope of the second target. In some embodiments, the VH CDR1, VH CDR2, VH CDR3, VL CDR1, VL CDR2 and VL CDR3 of the second binding domain form a binding site for an antigen of the second target. In some embodiments, the VH CDR1, VH CDR2, VH CDR3, VL CDR1, VL CDR2 and VL CDR3 of the second binding domain form a binding site for an epitope of the second target. In some embodiments, the second target is present on the surface of a target cell. In some embodiments, the target cell expressing the second target is killed when the multispecific TRGV9 antibody binds to TRGV9 on the surface of a T cell and the second target. In a specific embodiment, the T cell is a γδ T cell.

In some embodiments, the first binding domain of the multispecific TRGV9 antibody is multivalent. In some embodiments, the first binding domain of the multispecific TRGV9 antibody is capable of binding at least three antigens. In some embodiments, the first binding domain of the multispecific TRGV9 antibody is capable of binding at least four antigens. In some embodiments, the first binding domain of the multispecific TRGV9 antibody is capable of binding at least five antigens. In some embodiments, the second binding domain of the multispecific TRGV9 antibody is multivalent. In some embodiments, the second binding domain of the multispecific TRGV9 antibody is capable of binding at least three antigens. In some embodiments, the second binding domain of the multispecific TRGV9 antibody is capable of binding at least four antigens. In some embodiments, the second binding domain of the multispecific TRGV9 antibody is capable of binding at least five antigens.

In some embodiments, the first binding domain is humanized. In some embodiments, the second binding domain is humanized. In some embodiments, both the first binding domain and the second binding domain are humanized. In some embodiments, the multispecific TRGV9 antibody comprises a kappa light chain. In some embodiments, the multispecific TRGV9 antibody comprises a lambda light chain. In some embodiments, the multispecific TRGV9 antibody is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody.

In some embodiments, the multispecific TRGV9 antibody induces T cell dependent cytotoxicity of a target cell expressing the second target in vitro with an EC₅₀ of less than about 500 pM. In some embodiments, the multispecific TRGV9 antibody induces T cell dependent cytotoxicity of a target cell expressing the second target in vitro with an EC₅₀ of less than about 300 pM. In some embodiments, the multispecific TRGV9 antibody induces T cell dependent cytotoxicity of a target cell expressing the second target in vitro with an EC₅₀ of less than about 160 pM. In some embodiments, the EC₅₀ is assessed with a mixture of T cell effector cells and target cells expressing the second target. In some embodiments, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1. In a specific embodiment, the T cell is a γδ T cell. In one embodiment, target cells are Kasumi-3 AML target cells.

In another aspect, provided is a nucleic acid encoding a TRGV9 antibody provided herein. Also provided is a vector comprising a nucleic acid encoding a TRGV9 antibody provided herein. Also provided is a host cell comprising a vector comprising a nucleic acid encoding a TRGV9 antibody provided herein. Also provided is a kit comprising vector comprising a nucleic acid encoding a TRGV9 antibody provided herein, and packaging for the same. Also provided are methods of making a TRGV9 antibody provided herein, comprising culturing a cell comprising a nucleic acid encoding TRGV9 antibody under conditions to produce the TRGV9 antibody. In another aspect, provided is a kit comprising a TRGV9 antibody provided herein, and packaging for the same. In another aspect, provided is a pharmaceutical composition comprising a TRGV9 antibody provided herein, and a pharmaceutically acceptable carrier. In another aspect, provided is a method of producing a pharmaceutical composition comprising a TRGV9 antibody provided herein, comprising combining the TRGV9 antibody with a pharmaceutically acceptable carrier to obtain the pharmaceutical composition. In a specific embodiment, the TRGV9 antibody is a multispecific TRGV9 antibody provided herein.

In another aspect, provided is a multispecific TRGV9 antibody comprising: a first means capable of binding TRGV9; and a second means capable of binding a second target. In certain embodiments, the second target is not TRGV9. In another aspect, provided is a multispecific TRGV9 antibody comprising: a first means that binds to TRGV9; and a second means that binds to a second target. In certain embodiments, the second target is not TRGV9. In some embodiments, the TRGV9 is on the surface of a T cell. In certain embodiments, the T cell is a γδ T cell. In some embodiments, the first means is capable of specifically binding the TRGV9. In some embodiments, the first means specifically binds the TRGV9. In one embodiment, the TRGV9 is a TRGV9 antigen. In another embodiment, the TRGV9 is a TRGV9 epitope. In one embodiment, the first means is a paratope. In some embodiments, the paratope is a paratope of a TRGV9 antibody provided herein. In one embodiment, the first means is an antibody. In some embodiments, the antibody is TRGV9 antibody provided herein. In some embodiments, the TRGV9 antibody is an antigen binding fragment. In some embodiments, the second means is capable of specifically binding the second target. In some embodiments, the second means specifically binds the second target. In some embodiments, the second target is an antigen of the second target. In some embodiments, the second target is an epitope of the second target. In some embodiments, the second target is on the surface of a target cell. In some embodiments, the second target is CD123. In some embodiments, the second target is CD33. In some embodiments, the second target is TRBC1. In some embodiments, the second target is BCMA. In some embodiments, the second target is PSMA. In one embodiment, the second means is a paratope. In some embodiments, the paratope is a paratope of a second binding arm provided herein. In some embodiments, the paratope is a paratope of a CD123 antibody provided herein. In some embodiments, the paratope is a paratope of a CD33 antibody provided herein. In some embodiments, the paratope is a paratope of a TRBC1 antibody provided herein. In some embodiments, the paratope is a paratope of a BCMA antibody provided herein. In some embodiments, the paratope is a paratope of a PSMA antibody provided herein. In one embodiment, the second means is an antibody that binds a second target provided herein. In some embodiments, the second means is a CD123 antibody provided herein. In one embodiments, the CD123 antibody is an antigen binding fragment thereof. In some embodiments, the second means is a CD33 antibody provided herein. In one embodiments, the CD33 antibody is an antigen binding fragment thereof. In some embodiments, the second means is a TRBC1 antibody provided herein. In one embodiments, the TRBC1 antibody is an antigen binding fragment thereof. In some embodiments, the second means is a BCMA antibody provided herein. In one embodiments, the BCMA antibody is an antigen binding fragment thereof. In some embodiments, the second means is a PSMA antibody provided herein. In one embodiments, the PSMA antibody is an antigen binding fragment thereof.

In another aspect, provided is a process for making an antibody that binds to more than one target, the process comprising: a step for performing a function of obtaining a first binding domain capable of binding to TRGV9; a step for performing a function of obtaining a second binding domain capable of binding to a second target; and a step for performing a function of providing an antibody capable of binding to TRGV9 and the second target. In some embodiments, the step for performing a function of obtaining a second binding domain capable of binding to second target is repeated n times and further comprises n steps for performing a function of providing a first binding domain capable of binding to TRGV9 and n number of targets, wherein n is at least 2. In certain embodiments, the second target is not TRGV9. In some embodiments, the first binding domain is capable of specifically binding to the TRGV9. In one embodiment, the TRGV9 is a TRGV9 antigen. In another embodiment, the TRGV9 is a TRGV9 epitope. In one embodiment, the first binding domain binds a TRGV9 antigen. In one embodiment, the first binding domain binds a TRGV9 epitope. In some embodiments, the TRGV9 is on the surface of a T cell. In some embodiments, the T cell is a γδ T cell. In some embodiments, the second binding domain is capable of specifically binding to the second target. In one embodiment, the second binding domain binds an antigen of the second target. In one embodiment, the second binding domain binds an epitope of the second target. In some embodiments, the second target is on the surface of a target cell. In some embodiments, the second target is CD123. In some embodiments, the second target is CD33. In some embodiments, the second target is TRBC1. In some embodiments, the second target is BCMA. In some embodiments, the second target is PSMA. In one embodiment, the target cell is a cancer cell.

In another aspect, provided is a method of activating a T cell expressing TRGV9, comprising contacting the T cell with a TRGV9 antibody provided herein. In certain embodiments, the contacting results in an increase in CD69, CD25, and/or Granzyme B expression, as compared to a control T cell expressing TRGV9. In another aspect, provided is a method of inactivating a T cell expressing TRGV9, comprising contacting the T cell with an antibody that binds to a TRGV9 provided herein. Also provided is a method of blocking activation a T cell expressing TRGV9, comprising contacting the T cell with an antibody that binds to a TRGV9 provided herein. Also provided is a method of modulating the activity of a T cell expressing TRGV9, comprising contacting the T cell with an antibody that binds to a TRGV9 provided herein. In a specific embodiment, the T cell is a γδ T cell. In certain embodiments, the TRGV9 antibody is a multispecific TRGV9 antibody provided herein.

In another aspect provided is a method of directing a T cell expressing TRGV9 to a target cell, the method comprising contacting the T cell with a multispecific TRGV9 antibody provided herein, wherein the contacting directs the T cell to the target cell. In another aspect provided is a method of inhibiting growth or proliferation of a target cell, the method comprising contacting the target cell with a multispecific TRGV9 antibody provided herein, wherein the contacting inhibits growth or proliferation of the target cell. In some embodiments, the target cell is in the presence of the T cell expressing TRGV9 while in contact with the multispecific TRGV9 antibody. In some embodiments, the target cell expresses a second target that is not TRGV9. In some embodiments, the T cell is a γδ T cell. In some embodiments, the second target is an antigen of the second target. In some embodiments, the second target is an epitope of the second target. In some embodiments, the second target is on the surface of the target cell. In some embodiments, the second target is CD123. In some embodiments, the second target is CD33. In some embodiments, the second target is TRBC1. In some embodiments, the second target is BCMA. In some embodiments, the second target is PSMA. In one embodiment, the target cell is a cancer cell.

In another aspect provided is a method for eliminating target cells in a subject, comprising administering an effective amount of a multispecific TRGV9 antibody provided herein to the subject. In another aspect, provided is a method for treating a disease, disorder or condition (hereafter “disease”) caused all or in part by a target cell in a subject, comprising administering an effective amount of a multispecific TRGV9 antibody provided herein to the subject. In another aspect, provided is a method for preventing a disease caused all or in part by a target cell in a subject, comprising administering an effective amount of a multispecific TRGV9 antibody provided herein to the subject. In another aspect, provided is a method for modulating a disease caused all or in part by a target cell in a subject, comprising administering an effective amount of a multispecific TRGV9 antibody provided herein to the subject. In some embodiments, the target cell expresses a second target that is not TRGV9. In some embodiments, the second target is on the surface of the target cell. In some embodiments, the second target is CD123. In some embodiments, the second target is CD33. In some embodiments, the second target is TRBC1. In some embodiments, the second target is BCMA. In some embodiments, the second target is PSMA. In one embodiment, the target cell is a cancer cell. In one embodiment, the target cell is a T cell. In one embodiment, the target cell is a B cell. In one embodiment, the target cell is a dendritic cell. In one embodiment, the target cell is a NK cell. In one embodiment, the target cell is a stem cell. In one embodiment, the target cell is a stem cell precursor. In one embodiment, the target cell is a monocyte. In one embodiment, the target cell is a macrophage. In one embodiment, the target cell is a granulocyte. In one embodiment, the target cell is a platelet. In one embodiment, the target cell is an erythrocyte. In one embodiment, the target cell is an endothelial cell. In one embodiment, the target cell is an epithelial cell. In one embodiment, the second target is a pathogen. In one embodiment, the target cell is a cell comprising a pathogen. In one embodiment, the target cell is a blood cell. In one embodiment, the target cell is a myeloid cell. In some embodiments, the second target is on a cancer cell. In some embodiments, the target cell is a cancer cell. In a specific embodiment, the second target is on the surface of a cancer cell. In certain embodiments, the second target is an antigen on the surface of a cancer cell. In some embodiments, the antigen on the surface of the cancer cell is a tumor-specific antigen, a tumor-associated antigen, or a neoantigen In a specific embodiment, the disease is a cancer. In some embodiments, the subject is a subject in need thereof. In some embodiments, the subject is a human. In certain embodiments, the method further comprises identifying a subject in need thereof.

### BRIEF DESCRIPTION OF THE DRAWINGS

The foregoing summary, as well as the following detailed description of specific embodiments of the present application, will be better understood when read in conjunction with the appended drawings. It should be understood, however, that the application is not limited to the precise embodiments shown in the drawings.

FIG. 1 shows a schematic demonstrating the binding of an exemplary multispecific TRGV9 antibody comprising a first binding domain that binds to TRGV9 and a second binding domain that binds to a second target antigen on a target cell to recruit γδ T cells to the target cell and to induce target cell death. The second target antigen can be a tumor associated antigen (TAA), and the target cell can be a tumor cell, wherein the anti-TRGV9/anti-TAA bispecific antibody can to recruit γδ T cells to the cancer cell and to induce cancer cell death.

FIG. 2 shows a graph demonstrating that Zoledronic acid selectively expands Vγ9Vδ2 cells from whole peripheral blood mononuclear cells (PBMCs).

FIGS. 3A-3E show phenotypic characterization of Vγ9+ γδ T cells. FIG. 3A shows a schematic depiction of gates used to describe the differentiation of γδ T cells (left). Representative FACS-dot plots show the differentiation profile of Vγ9⁺γδ T cells from fresh PBMCs (left) and PBMCs cultured ex vivo with Zoledronic acid+IL-2+IL-15 for 14 days (right). Numbers in quadrants mirror the frequency (mean±SEM) of the respective population among fresh and activated Vγ9⁺γδ T cells. Represented data is mean (±SEM) of five donors (n=5) from a single experiment. FIG. 3B shows numbers in representative dot plots mirroring the frequency (mean±SEM) of Vγ9⁺γδ T cells positive for respective activation marker either from fresh PBMCs (upper row) or PBMCs cultured with Zoledronic acid+IL-2+IL-15 for 14 days (lower row). Represented data is mean (±SEM) of seven donors (n=7) for CD62L, CD69, CD44 expression data from two independent experiments. n=5 donors for NKG2D and 2 donors for CD45RO and CD71 expression data respectively from a single experiment. FIG. 3C shows numbers above gates in dot plots depicting the frequency (mean±SEM) of Vγ9⁺γδ T cells positive for respective inhibitory receptor surface expression either from fresh PBMCs (upper row) or PBMCs cultured with Zoledronic acid+IL-2+IL-15 for day 14 days (lower row). Data shown here is mean (±SEM) of five donors (n=5) for PD1, CTLA4, TIGIT and LAG3 surface expression and seven donors (n=7) for 2B4 and TIM3 surface expression data from two independent experiments. FIG. 3D shows representative FACS dot plots demonstrating the frequency (mean±SEM) of Vγ9⁺γδ T cells expressing intracellular Granzyme B (left column) and Perforin (right column) from fresh PBMCs (upper row) and PBMCs cultured ex vivo with Zoledronic acid+IL-2+IL-15 for 14 days (lower row). Depicted data is mean (±SEM) of four (n=4) and seven (n=7) donors for Granzyme B and Perforin data respectively from two independent experiments. FIG. 3E shows bars representing the mean (±SEM) concentration (pg/mL) of cytokine in the cell culture supernatant on day 0 and day 14 of PBMCs culture with Zoledronic acid+IL-2+IL-15. Represented data is mean (±SEM) of four wells (n=4) from a single donor.

FIG. 4 shows a histogram demonstrating that VG1 (an anti-TRGV9/anti-CD123 bispecific antibody) recruits Vγ9+ T cells as demonstrated by conjugate formation between γδ T cells and Kasumi-3 cells.

FIGS. 5A-5C show graphs demonstrating VG1 (anti-TRGV9/anti-CD123 bispecific antibody) bispecific mediated γδ T cell cytotoxicity against Kasumi-3 cells at different effector to target cell ratios (1:1 for FIG. 5A; 5:1 for FIG. 5B; and 10:1 for FIG. 5C).

FIGS. 6A-6C show graphs demonstrating CD69 (FIG. 6A), CD25 (FIG. 6B), or Granzyme B (FIG. 6C) expression on Vγ9+ γδ T cells, non-Vγ9+ γδ T cells, and Pan-T cells (non γδ T cells) co-cultured with Kasumi-3 cells and VG1, VG3, or no bispecific antibody.

FIG. 7 shows a schematic demonstrating the binding of an anti-TRGV9/anti-CD33 bispecific antibody to recruit γδ T-cells to a cancer cell that is CD33+ and to induce cancer cell death.

FIG. 8 shows an SDS-PAGE (non-reducing) gel demonstrating the integrity of the VG4 bispecific antibody.

FIG. 9 shows a graph demonstrating the binding of anti-CD33 antibody (clone C33B904) to MOLM-13 tumor cell line as measured by FACS.

FIG. 10 shows a graph demonstrating the binding of anti-CD33 antibody (clone C33B904) to Kasumi-1 tumor cell line as measured by FACS.

FIG. 11 shows a graph demonstrating the binding of anti-CD33 antibody (clone C33B904) to OCI-AML-3 tumor cell line as measured by FACS.

FIG. 12 shows a graph demonstrating that the anti-TRGV9/anti-CD33 bispecific antibody mediated γδ T cell cytotoxicity against CD33 expressing Kasumi-3 cells at 1:1 effector to target cell ratio. The effector cells were enriched γδ T cells isolated from PBMCs.

FIG. 13 shows a graph demonstrating that the anti-TRGV9/anti-CD33 bispecific antibody mediated γδ T cell cytotoxicity against CD33 expressing Kasumi-3 cells at 5:1 effector to target cell ratio. The effector cells were enriched γδ T cells isolated from PBMCs.

FIG. 14 shows a graph demonstrating that the anti-TRGV9/anti-CD33 bispecific antibody mediated γδ T cell cytotoxicity against CD33 expressing Kasumi-3 cells at 1:1 effector to target cell ratio. The effector cells were healthy donor derived PBMCs.

FIG. 15 shows a schematic demonstrating the binding of anti-TRGV9/anti-TRBC1 bispecific antibody to recruit γδ T-cells to a cancer cell that is TRBC1+ and to induce cancer cell death.

FIG. 16 shows selective cell binding of anti-TRBC1 (JOVI-1 mIgG2a, TRB1B1) to transfected Jurkat cells. The EC₅₀ for binding was ˜1 to 2 nM. TRB1B1 did not show any significant binding to HPB-ALL cell line that endogenously expresses TRBC2 TCR.

FIG. 17 shows selective protein binding of anti-TRBC1 (JOVI-1 mIgG2a, TRB1B1) to a recombinant TCR comprising of TRBC1 constant domain (TRB1W16). TRB1B1 did not show any significant binding to a recombinant TCR with TRBC2 constant domain (TRB2W16).

FIG. 18 shows phenotyping of Vγ9+ cells used for cytotoxicity studies of JOVI-1×Vg9 bispecific (TRB1B50) from a healthy donor.

FIG. 19 shows that the anti-TRGV9/anti-TRBC1 bispecific antibody mediates γδ T cell cytotoxicity against TRBC1 expressing Jurkat cells in vitro. Cytotoxicity values represented here were subtracted of basal cytotoxicity value observed in the absence of bispecific antibody. EC₅₀ values were calculated as described in methods. Representative data shown here are from a single experiment.

FIG. 20 shows bispecific antibody mediated cytotoxicity. Expanded and enriched Vγ9Vδ2 T cells from various donors were used to induce cytotoxicity to Jurkat cell line (E:T ratio 1:1) in presence of Vγ9×Jovi at indicated concentrations. Assay was conducted for 16 hrs. Percent dead target cells for various conditions are given in the figure.

FIG. 21 shows that the anti-TRGV9/anti-BCMA bispecific antibody (BCV9B106 (B3)) binds γδ T cells (left panel) and mediates γδ T cell cytotoxicity against BCMA expressing H929 cells in vitro (right panel). EC₅₀ values were calculated as described in methods. Representative data shown here are from a single experiment.

FIG. 22 shows that the anti-TRGV9/anti-BCMA bispecific antibody (HC1: VG9B420-LH-scFv; HC2: BCMA-Fab (BCMB519-Fab) (BCV9B71.001)) binds γδ T cells (left panel) and mediates γδ T cell cytotoxicity against BCMA expressing H929 cells in vitro (right panel). EC₅₀ values were calculated as described in methods. Representative data shown here are from a single experiment.

FIG. 23 shows that the anti-TRGV9/anti-BCMA bispecific antibody (VG9SB10SC1087_P18_D08-Fab RF, BCMA-scFv (BCMB519-scFv) (BCV9B100.001)) binds γδ T cells (left panel) and mediates γδ T cell cytotoxicity against BCMA expressing H929 cells in vitro (right panel). EC₅₀ values were calculated as described in methods. Representative data shown here are from a single experiment.

FIG. 24 shows that the anti-TRGV9/anti-BCMA bispecific antibody (VG9SB10SC1087_P18_C12-Fab RF, BCMA-scFv (BCMB519-scFv) (BCV9B101.001)) binds γδ T cells (left panel) and mediates γδ T cell cytotoxicity against BCMA expressing H929 cells in vitro (right panel). EC₅₀ values were calculated as described in methods. Representative data shown here are from a single experiment.

FIG. 25 shows that the anti-TRGV9/anti-BCMA bispecific antibody (VG9SB10SC1087_P19_C03-Fab RF, BCMA-scFv (BCMB519-scFv) (BCV9B103.001)) binds γδ T cells (left panel) and mediates γδ T cell cytotoxicity against BCMA expressing H929 cells in vitro (right panel). EC₅₀ values were calculated as described in methods. Representative data shown here are from a single experiment.

FIG. 26 shows humanization of murine anti-Vγ9 antibody. Humanization of murine clone 7A5 was performed following the process outlined by Singh et al., Mabs. 2015. 7:778-791. Based on sequence homology, germline IGHV1-8*01 and IGKV4-1*01 was chosen for framework adaption. A potential Iso-Asp isomerization site (DG motif) was also included in the design.

FIGS. 27A-27B show epitope and paratope mapping. FIG. 27A shows HX-MS epitope mapping for the mouse anti-human TCR Vγ9 [clone 7A5] mAb and Vγ9/Vδ2 fused to human Fc. Sequences of VG9 (SEQ ID NO:789 (amino acids 20-261 of SEQ ID NO:156)) and VD2 (SEQ ID NO:790 (amino acids 20-248 of SEQ ID NO:157)) are shown at the bottom. The peptide region comprising amino acids 49-68 of SEQ ID NO:789 (L₄₉VSISYDGTVRKESGIPSGK₆₈ (SEQ ID NO:774) (italicized)), was protected by mAb 7A5. A molecular model (using crystal structure PBD: 1HXM, see Allison et al., Nature. 2001. 411:820-824) of TCR Vγ9-Vδ2 and residues in the epitope are highlighted in the sphere representation. FIG. 27B shows HDX paratope mapping on the murine clone 7A5 (Vg9_7A5_VH SEQ ID NO:7; Vg9_7A5_VL SEQ ID NO:8). A molecular model of the Fab with residues in the paratope are highlighted.

FIGS. 28A-28B show healthy individuals harbor a wide range of Vγ9⁺ γδ T cells among whole PBMCs. FIG. 28A shows the frequency of Vγ9⁺ γδ T cells (TCRVγ9⁺ CD3⁺) cells among the whole PBMCs. Numbers in quadrants represent the frequency of respective population. FIG. 28B shows scatter dot plot graph summarizing the frequency (mean±SEM) of Vγ9⁺ γδ T cells among whole PBMCs of healthy individuals. Each dot represent data from a healthy individual.

FIGS. 29A-29C show characterisation of Vγ9⁺ γδ T cells. FIG. 29A, left and middle panels, show graphs summarizing the frequency of Vγ9+ γδ T cells among whole PBMCs on day 0 and day 14 of culture containing Zoledronic acid that selectively activates and expands Vγ9+ γδ T cells. FIG. 29A, right panel, shows representative data of n=15 donors from 6 independent experiments is shown in the graphs. Percent of Vγ9+ γδ T cells among total CD3+ T cells on day 0 and 14 of activation with each dot representing the data from a donor. FIG. 29B, left two panels, shows numbers in representative FACS plots depict the differentiation profile of Vγ9+ γδ T cells on day 0 and 14 of culture containing Zoledronic acid. FIG. 29B, right two panels, shows a scatter plot graph, which summarizes the frequency of Vγ9+ γδ T cells positive for Naïve (CD27+CD45RA+), Central memory (CD27+CD45RA−), Effector Memory (CD27− CD45RA−) and Effector Memory cells that re-expresses CD45RA (EMRA, CD27− CD45RA+) phenotypes on day 0 and 14 of culture containing Zoledronic acid. Each dot represents the data from a healthy donor. Representative data of n=13 donors from 4 independent experiments is shown in the plots. FIG. 29C shows numbers in representative FACS plots, which depict the frequency (mean±SEM) of Vγ9+ γδ T cells on day 0 (top row) and day 14 (bottom row) positive for Granzyme B and Perforin intracellular expression. Data for n=12 and n=7 donors from fresh Vγ9+ γδ T cells (Day 0) and for n=14 and n=9 donors from activated Vγ9+ γδ T cells (day 14) for Granzyme B and Perforin intracellular expression respectively is presented in the figure.

FIGS. 30A-30D show anti-TRGV9/anti-CD123 bispecific antibody binds to Vγ9+ γδ T cells and CD123 expressing tumor cells. CD123 expressing target cells and PBMCs that were activated and expanded with Zoledronic acid for day 14 were incubated in the presence or absence of indicated Vγ9 bispecific and Null arm control bispecific antibodies. Bound bispecific antibody staining was assessed by flow cytometry. FIG. 30A shows the frequency of Vγ9 bispecific bound Kasumi-3 cells in 3 independent experiments for Kasumi-3 cell line. The EC₅₀ values shown in the graphs refers to mean of 3 independent experiments. FIG. 30B shows Vγ9+ γδ T cells at various concentrations in 2 independent experiments with 8 healthy donors. EC₅₀ values shown in the graphs refers to 2 healthy donors. Right and left lines reflect the indicated Vγ9 bispecific antibody and its corresponding Vγ9 null arm bispecific control antibodies respectively. EC₅₀ values shown in the graph were derived using a 4-parameter dose-response curve with the concentration of indicated bispecific antibody on the x-axis (log scale) and specific binding on the y-axis (linear scale). FIG. 30C shows Vγ9+ γδ T cells were FACS-sort depleted from Pan-T cells of whole PBMCs. Total Pan-T cells and Pan-T cells depleted of Vγ9+ γδ T cells were incubated in the presence and absence of indicated bispecific antibodies at various concentrations. Representative FACS plots show the depletion efficacy of Vγ9+ γδ T cells among Pan-T cells. Numbers in quadrants represent the frequency of the respective population. The binding of Vγ9/CD123 and Vγ9/NULL bispecific antibodies are shown at indicated concentrations to pan-T cells (Vγ9 non-depleted Pan-T cells) and pan-T cells depleted of Vγ9+ γδ T cells (Vγ9 depleted Pan-T cells). FIG. 30D shows CD123 expressing Kasumi-3 and non-expressing 22Rv1 cell lines were stained with anti-CD123 monoclonal antibody. The representative overlaid histogram show the staining of CD123, isotype control and FMO control on Kasumi-3 (left) and 22Rv1 (right) cell lines (upper panels). At the bottom, the representative graphs show the binding of Vγ9/CD123 and Vγ9/NULL bispecific antibodies to Kasumi-3 (left) cells lines at indicated concentrations. EC₅₀ values shown in the graph were derived using a 4-parameter dose-response curve with the concentration of indicated bispecific antibody on the x-axis (log scale) and specific binding on the y-axis (linear scale).

FIGS. 31A-31F shows that Vγ9/CD123 bispecific antibody selectively recruits, activates and induces cytotoxicity mediated by Vγ9+ γδ T cells. FIG. 31A shows cell trace labelled enriched γδ T cells that were co-cultured with cell trace yellow labelled kasumi-3 cells at 1:1 ET ratio in the presence of 1 μg/mL of indicated bispecific antibody at 37° C. for 1 hour. Cell-cell association was determined using flow cytometry and quantified as double positive, cells in upper right quadrant of FACS plot. Numbers in quadrants indicate the frequency of respective population. FIGS. 31B and 31C show Pan T cells (effectors) from fresh PBMCs that were co-cultured with Kasumi-3 cells (Targets) at 1:1 ET ratio in the presence or absence of indicated bispecific antibodies at 37° C. for 72 hours. Vγ9+ γδ T cells, Vγ9− γδ T cells and Non-γδ T cells that were positive for CD69 (left), CD25 (right) surface expression and intra cellular Granzyme B expression are shown. FIG. 31D shows Vγ9/CD123 bispecific mediated γδ T cells cytotoxicity against CD123 cells. PBMCs cultured with Zoledronic acid for 14 of (effectors) were co-cultured with CFSE labelled target (Kasumi-3) cells at ET ratio 1:1 (by normalizing ET ratio to Vγ9+γ6 T cell frequency in expanded PBMCs) in the presence of indicated concentration of Vγ9 bispecifics and Vγ9 NULL arm control antibodies for a period of 16 hours. Target cell lysis was determined by the 7-AAD staining and flow cytometry. Graphs represent the frequency of specific target cell lysis at the indicated of concentration of Vγ9 bispecific antibodies and their respective Vγ9/NULL arm controls. EC₅₀ values shown in representative graphs are mean of 8 healthy donors for Vγ9/CD123 from 3 independent experiments. FIG. 31E shows Vγ9/CD123 bispecific effectively mediates AML γδ T cells cytotoxicity against Kasumi-3 cells. The upper and the lower lines in representative graphs show the frequency of target (kasumi-3) cell lysis (%7-AAD⁺ cells) mediated by Vγ9/CD123 and Vγ9/Null bispecific antibodies respectively, upon co-culture of day 14 Zol AML patient PBMCs with target cells for 16 hours. FIG. 31F shows the depletion efficacy of Vγ9⁺γδ T cells among pan-T cells. Numbers in quadrants represent the frequency of the respective population. Graphs show the frequency of target cell lysis (% 7-AAD⁺ cells) mediated by Vγ9/CD123 and Vγ9/NULL bispecific antibodies respectively at indicated concentrations, upon co-culture of pan-T cells (Vγ9 non-depleted) and pan-T cells depleted of Vγ9⁺γδ T cells (Vγ9 depleted) with target (Kasumi-3) cells (middle graph). Bottom graph shows the target cell lysis mediated by CD3/CD123 and CD3/NULL bispecific antibodies respectively at indicated concentrations.

FIGS. 32A-32C show Vγ9/CD123 bispecific antibody potently mediates activation, proliferation and effector functions of Vγ9⁺ γδ T cells among whole PBMCs. CFSE labelled whole PBMCs were cultured in the presence or absence kasumi-3 cells in the presence plus indicated bispecific antibodies at a concentration of 3 ng/mL. FIG. 32A shows frequency of Vγ9⁺ cells positive for surface expression of CD69 and CD25. FIG. 32B shows CFSE dilution (proliferation profile). FIG. 32C shows the ability to eliminate exogenously added Kasumi-3 cells. Each dot represent data from an individual donor. Representative data of n=5 donors from 2 independent experiments is shown in here.

FIG. 33 shows Vγ9+ γδ T cell selective redirection does not elicit cytokine storm compared to Pan-T cell re-direction. Whole PBMCs were cultured in the presence or absence of spiked-in kasumi-3 cells in the presence or absence of indicated bispecific antibodies (3 ng/ml) as described in FIG. 30. From day 3 of culture onwards, 100 μL of culture medium was removed every day from the wells, without disturbing the cells, and replenished with fresh medium until day 8 of culture. Cytokines were assessed from day 3 to day 8 cell culture supernatant. FIG. 33 shows concentration of various cytokines or effector molecules in the culture supernatant of whole PBMCs stimulated with indicated bispecific antibodies. Circles and squares represent PBMCs from four individual donors stimulated with indicated bispecific antibodies or NULL arm control bispecific antibodies respectively. Representative data of n=4 donors from one independent experiment is shown here.

FIG. 34 shows mean tumor growth kinetics of NOD SCID mice bearing subcutaneous KG-1 tumor xenograft. Female NOD SCID mice inoculated subcutaneously with 1.5×10⁶ KG-1 cells were weekly treated subcutaneously with PBS or Vγ9Vδ2 γδ T cells. All mice received 15 μg/kg IL-2, and, as indicated, 1. 5 mg/kg (Vβ17×DLL3). Tumor volume (left) of each mouse was measured once every three days during experimental period. Values are expressed as Mean±SEM of 6 animals in each group. Statistical analysis carried out by Two-way ANOVA followed by Bonferroni post tests using Graph Pad Prism (Version 8.3.0). * p<0.05 & **** p<0.0001 when respective test groups were compared to Group 1−Tumor+PBS (control) group.

FIG. 35 shows binding kinetics of mouse anti-human TCR Vγ9 [clone 7A5] and recombinant Vγ9-Vδ2-Fc antigen by SPR at 25° C. Different concentrations of antigen (100 nM, from top to bottom in the plot) were flowed through anti-Vγ9 mAb that was captured on the surface. Experimental data (black dotted line) and 1:1 Langmuir binding fitting (smooth line) is shown. The association phase between (first ˜250 sec) is follow by the dissociation phase. Global fitting to a 1:1 simple Langmuir binding model resulted kₒₙ=1.3±0.2×10⁵ M⁻¹ S⁻¹ and k_{off}=2.43±0.3×10⁻⁴ S⁻¹ giving a K_{D}=1.9 nM.

FIGS. 36A-36D show that Vγ9+ subset of γδ T cell are suitable for redirecting for tumor elimination. FIG. 36A shows the frequency (mean±SEM) of ζγ9+ γδ T cells on day 0 (top row) and day 14 (bottom row) positive for activation markers. FIG. 36B shows an antigen presenting cell characteristics. FIG. 36C shows exhaustion markers. FIG. 36D shows NK markers/characteristics. Representative data on n=7 donors for CD62L, CD69, CD44, 2 donors for CD45RO and CD71, 3 donors for CD86, HLA-DR and CD16, 5 donors for NKG2D, 3 donors for CD95 (Fas) surface expression on day 0 Vγ9+ γδ T cells. n=8 donors for CD62L, 9 donors for CD69 and CD44, 5 donors for CD45Ro and CD71, 3 donors for CD86, HLA-DR, CD16, 14 donors for NKG2D, 6 donors for CD95 (Fas) surface expression on activated Vγ9+ γδ T cells (day 14 n=13 donors for PD1 and Lag3, 5 donors for CTLA4 and 2B4, 4 and 7 donors for TIGIT and Tim3 surface expression respectively on fresh Vγ9+ γδ T cells (day 0). n=16 donors for PD1, 5 donors for CTLA4 and 2B4, 13 donors for Lag3, 14 donors for TIGIT surface expression on activated Vγ9+ γδ T cells (day 14). >5 individual experiments were carried out.

FIGS. 37A-37B show Vγ9 bispecific binding to Vγ9⁺γδ T cells and TAA expressing tumor cells. Tumor Associated Antigen (TAA) expressing target cells and day 14 Zol expanded PBMCs were incubated in the presence or absence of indicated Vγ9 bispecific (Vγ9/CD123 or Vγ9/PSMA) and Null arm control bispecific antibodies. Bound bispecific antibody staining was assessed by flow cytometry. Representative graphs show the frequency of Vγ9 bispecific bound cells at various concentrations. Vγ9 bispecific antibody and its corresponding Vγ9 null arm bispecific control antibodies are indicated. EC₅₀ values shown in the graph were derived using a 4-parameter dose-response curve with the concentration of indicated bispecific antibody on the x-axis (log scale) and specific binding on the y-axis (linear scale). FIG. 37A shows representative data of n=3 independent experiments for Kasumi-3, 22Rv1 cell lines and FIG. 37B shows representative data of n=8 and 2 healthy donors from 2 independent experiments are shown for Vγ9/CD123 and Vγ9/PSMA bispecifics respectively. EC₅₀ values shown in the graphs refers to mean of 3 independent experiments (for FIG. 37A) and 9 and 2 healthy donors (for FIG. 37B).

FIGS. 38A-38B show that Vγ9/CD123 bispecific selectively binds to Vγ9⁺γδ T cells and CD123 expressing cell line. Vγ9⁺γδ T cells were FACS-sort depleted from enriched Pan-T cells of whole PBMCs from healthy individuals. Total Pan-T cells and Pan-T cells depleted of Vγ9⁺γδ T cells were incubated in the presence and absence of indicated bispecific antibodies at various concentrations. FIG. 38A depicts representative FACS plots showing the depletion efficacy of Vγ9⁺γδ T cells among pan-T cells. Numbers in quadrants represent the frequency of the respective population. FIG. 38A reflects the binding of Vγ9/CD123 and Vγ9/NULL bispecific antibodies at indicated concentrations to pan-T cells (Vγ9 non-depleted Pan-T cells) and pan-T cells depleted of Vγ9⁺γδ T cells (Vγ9 depleted Pan-T cells). CD123 TAA expressing Kasumi-3 and non-expressing 22Rv1 cell lines were stained with anti-CD123 monoclonal antibody. The upper panels of FIG. 38B show the staining of CD123, isotype control and FMO control respectively on Kasumi-3 (left) and 22Rv1 (right) cell lines. The bottom panels of FIG. 38B show the binding of Vγ9/CD123 and Vγ9/NULL bispecific antibodies respectively to Kasumi-3 (left) and 22Rv1 (right) cells lines at indicated concentrations. EC₅₀ values shown in the graph were derived using a 4-parameter dose-response curve with the concentration of indicated bispecific antibody on the x-axis (log scale) and specific binding on the y-axis (linear scale).

FIGS. 39A-39B show that Vγ9 bispecific mediated re-direction of γδ T cells effectively eliminates liquid and solid tumors. Whole PBMCs were cultured in the presence of Zol+IL-2+Il-15 for 14 days. Vγ9⁺γδ T cell frequency was assessed among whole PBMCs by flow cytotmetry. Day 14 Zol cultured PBMCs (effectors) were co-cultured with CFSE labelled target (Kasumi-3) cells at ET ratio 1:1 (for Kasumi-3 cells) and 5:1 (for 22Rv1) ET ratio (by normalizing ET ratio to Vγ9 frequency in Zol expanded PBMCs) in the presence of indicated concentration of Vγ9 bispecifics and Vγ9 NULL arm control antibodies for a period of 16 hours (for Kasumi-3 targets) and 72 hours (for 22Rv1) and at 37° C. in a humidified CO2 incubator. Target cell lysis was determined by the 7-AAD staining and flow cytometry. Graphs shown in FIG. 39A and FIG. 39B represent the frequency of specific target cell lysis at the indicated concentration of Vγ9 bispecific antibodies and their respective Vγ9/NULL arm controls. EC₅₀ values shown in representative graphs are mean of 8 and 2 healthy donors for Vγ9/CD123 (FIG. 39A) and Vγ9/PSMA (FIG. 39B) bispecific antibodies respectively from 3 (for FIG. 39A) and one (for FIG. 39B) independent experiments.

FIGS. 40A-40E show that Vγ9/CD123 bispecific antibody potently mediates activation, proliferation and effector functions of Vγ9⁺ γδ T cells among whole PBMCs. FIG. 40A shows how CFSE labelled whole PBMCs were cultured in the presence or absence of spiked-in kasumi-3 cells in the presence of indicated bispecific antibodies at a concentration of 3 ng/mL. As a control, CFSE labelled whole PBMCs (with or without spiked in Kasumi-3 cells) were cultured in the absence of any bispecific antibody. FIG. 40B depicts graphs that represent the mean (±SEM) frequency of Vγ9⁺ cells positive for surface expression of CD69, CD25 and CD71 (activation markers). FIG. 40C shows CFSE dilution (proliferation profile) and FIG. 40D shows ability to eliminate exogenously added Kasumi-3 cells or endogenous CD123⁺ cells (as shown in FIG. 40E) among whole PBMCs (effector profile) upon culture in the absence or presence of indicated bispecific antibodies (FIGS. 40C-40E). Each dot represent data from an individual healthy donor. Representative data of n=5 donors from 2 independent experiments is shown in here.

FIGS. 41A-41C show that Vγ9+ γδ T cell selective redirection do not elicit cytokine storm compared to Pan-T cell re-direction. Whole PBMCs were cultured in the presence or absence of spiked-in kasumi-3 cells in the presence or absence of indicated bispecific antibodies (3 ng/ml) as described in FIG. 40. From day 3 of culture onwards, 100 μL of culture medium was removed every day from the wells, without disturbing the cells, and replenished with fresh medium until day 8 of culture. Cytokines were assessed from day 3 to day 8 cell culture supernatant. FIG. 41A, FIG. 41B and FIG. 41C show concentration of various cytokines or effector molecules in the culture supernatant of whole PBMCs stimulated with indicated bispecific antibodies. Circles and squares represent PBMCs from four individual donors stimulated with indicated bispecific antibodies or NULL arm control bispecific antibodies respectively. Representative data of n=4 donors from one independent experiment is shown here.

FIGS. 42A-42D show Vγ9/CD123 bispecific mediated γδ T cells redirection in AML patients PBMCs. FIG. 42A shows TCR Vγ9⁺γδ T cells from AML patients can be expanded via ZoL. Numbers in representative FACS plots show the frequency of Vγ9⁺ and Vγ9⁻γδ T cells on day 0 (left) and day 14 (right) AML patients PBMCs culture with Zol+IL-2+IL-15. FIG. 42B shows the fold of expansion of TCRVγ9⁺γδ T cells from four AML patients PBMCs. FIG. 42C shows TCR Vγ9⁺γδ T cells from LC patients PBMCs exhibit more activated phenotype. Scatter dot plot graphs shows the frequency of Vγ9⁺γδ T cells, from fresh PBMCs, positive for naïve (CD27⁺CD45RA⁺), Central Memory (T_{CM}: CD27⁺ CD45RA⁻), Effector Memory (T_{EM}: CD27⁻CD45RA⁻) and Effector Memory cells that re-express CD45RA (EMRA: CD27⁻CD45RA⁺) phenotype. Each dot represented data from an a Lung Cancer patient sample. FIG. 42D shows Vγ9/CD123 bispecific effectively mediates AML γδ T cells cytotoxicity against Kasumi-3 cells. It shows the frequency of target (kasumi-3) cell lysis (%7-AAD⁺ cells) mediated by Vγ9/CD123 and Vγ9/Null bispecific antibodies, upon co-culture of day 14 Zol cultured healthy (left) or AML patient PBMCs (middle and right) with target cells for 16 hours. No bispecific control well values were subtracted from bispecific wells.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., anti-TRGV9/anti-cancer-associated antigen bispecific antibodies and polynucleotides that encode them, anti-TRGV9/anti-CD123 bispecific antibodies and polynucleotides that encode them, TRGV9 polypeptides and TRGV9 polynucleotides that encode them, CD123 polypeptides and CD123 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed bispecific antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

Antibodies

Provided herein are TRGV9 antibodies, nucleic acids and expression vectors encoding the antibodies, recombinant cells containing the vectors, and compositions comprising the antibodies. In certain embodiments, provided are isolated TRGV9 antibodies, nucleic acids and expression vectors encoding the antibodies, recombinant cells containing the vectors, and compositions comprising the antibodies. Methods of making the antibodies, and methods of using the antibodies to treat diseases are also provided. The antibodies disclosed herein possess one or more desirable functional properties, including but not limited to high-affinity binding to TRGV9 or high specificity to TRGV9. In certain embodiments, the antibodies disclosed herein possess the ability to treat or prevent a disease or disorder when administered to a subject alone or in combination with other therapies. In certain embodiments, the TRGV9 antibody comprises a TRGV9 antigen binding fragment. In some embodiments, the TRGV9 antibody consists of a TRGV9 antigen binding fragment. In other embodiments, the TRGV9 antibody is a multispecific TRGV9 antibody. In yet other embodiments, the multispecific TRGV9 antibody is a bispecific TRGV9 antibody. While TRGV9 antibodies are exemplified herein, it is understood that other molecules that bind to TRGV9 are also contemplated. Such molecules include other alternative binding agents, including equivalents of the antibodies and other antibody binding fragments provided herein.

Also provided herein are TRGV9 multispecific antibodies, nucleic acids and expression vectors encoding the multispecific antibodies, recombinant cells containing the vectors, and compositions comprising the multispecific antibodies. Methods of making the antibodies, and methods of using the multispecific antibodies to treat diseases, including cancer, are also provided. The antibodies disclosed herein possess one or more desirable functional properties. In some embodiments, the multispecific antibodies provided herein have high-affinity binding to TRGV9. In some embodiments, the multispecific antibodies provided herein have high-affinity binding to a second target antigen. In some embodiments, the multispecific antibodies provided herein have high specificity to TRGV9. In some embodiments, the multispecific antibodies provided herein have high specificity to a second target antigen. In some embodiments, the multispecific antibodies provided herein have the ability to treat or prevent a disease or disorder when administered alone. In some embodiments, the multispecific antibodies provided herein have the ability to treat or prevent a disease or disorder when administered in combination with other therapies. In a specific embodiment, the multispecific antibody is a bispecific antibody. In some embodiments, the TRGV9 antibody comprise an antigen binding fragment thereof.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. In specific embodiments, the antibodies provided herein are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies provided herein can contain a kappa or lambda light chain constant domain. According to particular embodiments, the antibodies of the invention include heavy and/or light chain constant regions from rat or human antibodies.

In addition to the heavy and light constant domains, antibodies contain an antigen-binding region that is made up of a light chain variable region (VL) and a heavy chain variable region (VH), each of which contains three domains (i.e., complementarity determining regions 1 (CDR1), CDR2 and CDR3. A “CDR” refers to one of three hypervariable regions (HCDR1, HCDR2 or HCDR3) within the non-framework region of the immunoglobulin (Ig or antibody) VH β-sheet framework, or one of three hypervariable regions (LCDR1, LCDR2 or LCDR3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. CDR region sequences have also been defined by AbM, Contact and IMGT. Exemplary CDR region sequences are illustrated herein, for example, in the Sequence Listing, and tables provided in the Examples below. The positions of CDRs within a canonical antibody variable region have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra (1997)). Such nomenclature is similarly well known to those skilled in the art.

The light chain variable region CDR1 domain is interchangeably referred to herein as LCDR1 or VL CDR1. The light chain variable region CDR2 domain is interchangeably referred to herein as LCDR2 or VL CDR2. The light chain variable region CDR3 domain is interchangeably referred to herein as LCDR3 or VL CDR3. The heavy chain variable region CDR1 domain is interchangeably referred to herein as HCDR1 or VH CDR1. The heavy chain variable region CDR2 domain is interchangeably referred to herein as HCDR2 or VH CDR2. The heavy chain variable region CDR1 domain is interchangeably referred to herein as HCDR3 or VH CDR3.

The term “hypervariable region”, such as a VH or VL, when used herein refers to the regions of an antibody variable region that are hypervariable in sequence and/or form structurally defined loops. Generally, antibodies comprise six hypervariable regions; three in the VH (HCDR1, HCDR2, HCDR3), and three in the VL (LCDR1, LCDR2, LCDR3). A number of hypervariable region delineations are in use and are encompassed herein. The “Kabat” CDRs are based on sequence variability and are the most commonly used (see, e.g., Kabat et al., Sequences of Proteins of Immunological Interest, 5th Ed. Public Health Service, National Institutes of Health, Bethesda, Md. (1991)). “Chothia” refers instead to the location of the structural loops (see, e.g., Chothia and Lesk, J. Mol. Biol. 196:901-917 (1987)). The end of the Chothia CDR-HCDR1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The “AbM” hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). “Contact” hypervariable regions are based on an analysis of the available complex crystal structures.

Recently, a universal numbering system has been developed and widely adopted, ImMunoGeneTics (IMGT) Information System® (Lafranc et al., Dev. Comp. Immunol. 27(1):55-77 (2003)). IMGT is an integrated information system specializing in immunoglobulins (IG), T cell receptors (TR) and major histocompatibility complex (MEW) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Pluckthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (LCDR1), 46-56 or 50-56 (LCDR2) and 89-97 or 89-96 (LCDR3) in the VL and 26-35 or 26-35A (HCDR1), 50-65 or 49-65 (HCDR2) and 93-102, 94-102, or 95-102 (HCDR3) in the VH. CDR sequences, reflecting each of the above numbering schemes, are provided herein, including in the Sequence Listing.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region may contain the CH1, CH2 and CH3 regions of the heavy chain and the CL region of the light chain.

The term “framework” or “FR” residues are those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

As used herein, the term an “isolated antibody” refers to an antibody which is substantially free of other antibodies having different antigenic specificities (e.g., an isolated antibody that specifically binds to TRGV9 is substantially free of antibodies that do not bind to Vγ9; an isolated antibody that specifically binds to a second target (e.g., CD123) is substantially free of antibodies that do not bind to the second target (e.g., CD123). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope (e.g., an epitope on a TRGV9 antigen) and a second immunoglobulin variable domain sequence that has binding specificity for a second epitope. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on TRGV9 and the second epitope is located on CD123. In an embodiment, the first epitope is located on TRGV9 and the second epitope is located on CD33. In an embodiment, the first epitope is located on TRGV9 and the second epitope is located on TRBC1. In an embodiment, the first epitope is located on TRGV9 and the second epitope is located on BCMA. In an embodiment, the first epitope is located on TRGV9 and the second epitope is located on PSMA. In an embodiment, the first epitope is located on TRGV9 and the second epitope is located on PD-1, PD-L1, CTLA-4, EGFR, HER-2, CD19, CD20, CD3 and/or other cancer associated immune suppressors or surface antigens.

The term “half antibody” as used herein refers to one immunoglobulin heavy chain associated with one immunoglobulin light chain. An exemplary half-antibody is depicted in SEQ ID NO:17. One skilled in the art will readily appreciate that a half-antibody can encompass a fragment thereof and can also have an antigen binding domain consisting of a single variable domain, e.g., originating from a camelidae.

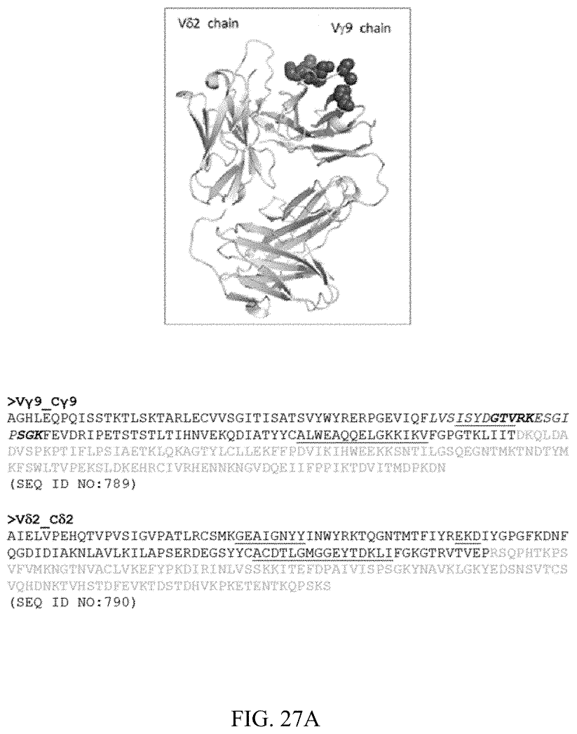

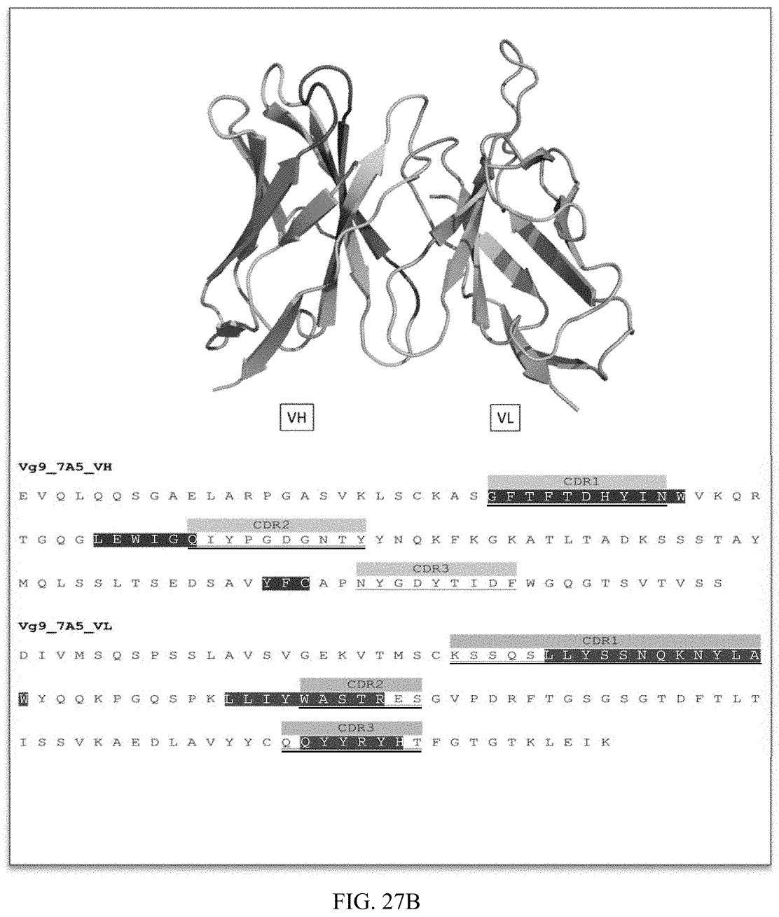

As used herein, the term “TRGV9” refers to a polypeptide capable of forming a T cell receptor when expressed on the surface of γδ T cells. TRGV9-expressing γδ T cells are among the first T cells to develop in the human fetus and are the predominant γδ T cell subset in healthy adult peripheral blood cells. The term “TRGV9” includes any TRGV9 variant, isoform, and species homolog, which is naturally expressed by cells (including T cells) or can be expressed on cells transfected with genes or cDNA encoding the polypeptide. In specific embodiments, the TRGV9 is a human TRGV9. An exemplary human TRGV9 amino acid sequence is provided by GenBank Accession Number NG 001336.2. An exemplary human TRGV9 is also provided in FIG. 27A and SEQ ID NO:789.

The term “CD123” refers to a molecule that is found on cells which helps transmit the signal of interleukin-3, a soluble cytokine that is important in the immune system. CD123 can also be referred to as the “interleukin-3 receptor.” The receptor belongs to the type I cytokine receptor family and is a heterodimer with a unique alpha chain paired with the common beta subunit (beta c or CD131). The CD123 receptor can be found on pluripotent progenitor cells and can induce tyrosine phosphorylation within the cell and promote proliferation and differentiation within hematopoietic cell lines. CD123 can also be expressed in acute myeloid leukemia (AML) subtypes. The term “CD123” includes any CD123 variant, isoform, and species homolog, which is naturally expressed by cells (including T cells) or can be expressed on cells transfected with genes or cDNA encoding those polypeptides, unless noted, in specific embodiments the “CD123” is a human CD123. A human CD123 amino acid sequence is provided by GenBank Accession Number AY789109.1.

The term “CD33” refers to a 67 kD single pass transmembrane glycoprotein and is a member of the sialic acid-binding immunoglobulin-like lectins (Siglecs) family. While its exact biological function is unclear, in normal individuals, it is primarily considered to be a myeloid differentiation antigen, with low expression in myeloid progenitors, neutrophils and macrophages while being highly expressed in circulating monocytes and dendritic cells. CD33 has been detected on blasts and leukemic stem cells of 85-90% of patients presenting with in acute myeloid leukemia (AML). The term “CD33” includes any CD33 variant, isoform, and species homolog, which is naturally expressed by cells or can be expressed on cells transfected with genes or cDNA encoding those polypeptides, unless noted, the “CD33” is a human CD33. A human CD33 amino acid sequence is provided by GenBank Accession Number BC028152.1.

As used herein, an antibody that “specifically binds” to a target refers to an antibody that binds to a target with a KD of 1×10⁻⁷M or less, such as 1×10⁻⁸M or less, 5×10⁻⁹ M or less, 1×10⁻⁹M or less, 5×10⁻¹⁰ M or less, or 1×10⁻¹⁰ M or less. In specific embodiments, the target is a human target. The target can be, e.g., TRGV9, CD123, CD33, TRBC1, BCMA, or PSMA.

The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD of an antibody, the higher affinity that the antibody binds to a target antigen.

In one aspect, provided herein is an antibody that binds to TRGV9. In some embodiments, the antibody comprises a heavy chain variable (VH) region and a light chain variable (VL) region. In a some embodiments, the TRGV9 antibody is not a single domain antibody or nanobody. In some embodiments, the TRGV9 antibody is a humanized antibody.

In certain embodiments, provided herein is a TRGV9 antibody comprising a VH region, VL region, VH CDR1, VH CDR2, VH CDR3, VL CDR1, VL CDR2, and/or VL CDR3 of any one of the antibodies described herein. In some embodiments, provided herein is a TRGV9 antibody comprising a VH region of any one of the antibodies described herein. In some embodiments, provided herein is a TRGV9 antibody comprising a VL region of any one of the antibodies described herein. In some embodiments, provided herein is a TRGV9 antibody comprising a VH region of any one of the antibodies described herein, and a VL region of any one of the antibodies described herein. In some embodiments, provided herein is a TRGV9 antibody comprising a VH CDR1, VH CDR2, and VH CDR3 of any one of the antibodies described herein. In some embodiments, provided herein is a TRGV9 antibody comprising a VL CDR1, VL CDR2, and VL CDR3 of any one of the antibodies described herein. In some embodiments, provided herein is a TRGV9 antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of TRGV9 antibodies provided herein are provided in the Sequence Listing, as well as Tables 1-39.

In some embodiments, the TRGV9 antibody is a multispecific TRGV9 antibody provided herein. In some embodiments, the multispecific TRGV9 antibody is a bispecific TRGV9 antibody. In one embodiment, the multispecific TRGV9 antibody comprises: (a) a first binding domain that binds to TRGV9, and (b) a second binding domain that binds to a second target that is not TRGV9.

In certain embodiments, the first binding domain that binds to TRGV9 comprises a VH region, VL region, VH CDR1, VH CDR2, VH CDR3, VL CDR1, VL CDR2, and/or VL CDR3 of any one of the TRGV9 antibodies described herein. In some embodiments, the first binding domain that binds to TRGV9 comprises a VH region of any one of the TRGV9 antibodies described herein. In some embodiments, the first binding domain that binds to TRGV9 comprises a VL region of any one of the TRGV9 antibodies described herein. In some embodiments, the first binding domain that binds to TRGV9 comprises a VH region and a VL region of any one of the TRGV9 antibodies described herein. In some embodiments, the first binding domain that binds to TRGV9 comprises a VH CDR1, VH CDR2, and VH CDR3 of any one of the TRGV9 antibodies described herein. In some embodiments, the first binding domain that binds to TRGV9 comprises a VL CDR1, VL CDR2, and VL CDR3 of any one of the TRGV9 antibodies described herein. In some embodiments, the first binding domain that binds to TRGV9 comprises a VH CDR1, VH CDR2, VH CDR3, VL CDR1, VL CDR2, and VL CDR3 of any one of the TRGV9 antibodies described herein. Representative VH and VL amino acid sequences, including VH CDR1, VH CDR2, VH CDR3, VL CDR1, VL CDR2 and VL CDR3 amino acid sequences, of TRGV9 antibodies provided herein are provided in the Sequence Listing, as well as Tables 1-39.

In some embodiments, the second target is CD123. In some embodiments, second binding domain that binds CD123 has a VH region, VL region, VH CDR1, VH CDR2, VH CDR3, VL CDR1, VL CDR2, and/or VL CDR3 of a CD123 antibody provided herein. In some embodiments, second binding domain that binds CD123 has a VH region of a CD123 antibody provided herein. In some embodiments, second binding domain that binds CD123 has a VL region of a CD123 antibody provided herein. In some embodiments, second binding domain that binds CD123 has a VH region and a VL region of a CD123 antibody provided herein. In some embodiments, second binding domain that binds CD123 has a VH CDR1, VH CDR2, and VH CDR3 of a CD123 antibody provided herein. In some embodiments, second binding domain that binds CD123 has a VL CDR1, VL CDR2, and VL CDR3 of a CD123 antibody provided herein. In some embodiments, second binding domain that binds CD123 has a VH CDR1, VH CDR2, VH CDR3, VL CDR1, VL CDR2, and VL CDR3 of a CD123 antibody provided herein.

In some embodiments, the second target is CD33. In some embodiments, second binding domain that binds CD33 has a VH region, VL region, VH CDR1, VH CDR2, VH CDR3, VL CDR1, VL CDR2, and/or VL CDR3 of a CD33 antibody provided herein. In some embodiments, second binding domain that binds CD33 has a VH region of a CD33 antibody provided herein. In some embodiments, second binding domain that binds CD33 has a VL region of a CD33 antibody provided herein. In some embodiments, second binding domain that binds CD33 has a VH region and a VL region of a CD33 antibody provided herein. In some embodiments, second binding domain that binds CD33 has a VH CDR1, VH CDR2, and VH CDR3 of a CD33 antibody provided herein. In some embodiments, second binding domain that binds CD33 has a VL CDR1, VL CDR2, and VL CDR3 of a CD33 antibody provided herein. In some embodiments, second binding domain that binds CD33 has a VH CDR1, VH CDR2, VH CDR3, VL CDR1, VL CDR2, and VL CDR3 of a CD33 antibody provided herein.

In some embodiments, the second target is TRBC1. In some embodiments, second binding domain that binds TRBC1 has a VH region, VL region, VH CDR1, VH CDR2, VH CDR3, VL CDR1, VL CDR2, and/or VL CDR3 of a TRBC1 antibody provided herein. In some embodiments, second binding domain that binds TRBC1 has a VH region of a TRBC1 antibody provided herein. In some embodiments, second binding domain that binds TRBC1 has a VL region of a TRBC1 antibody provided herein. In some embodiments, second binding domain that binds TRBC1 has a VH region and a VL region of a TRBC1 antibody provided herein. In some embodiments, second binding domain that binds TRBC1 has a VH CDR1, VH CDR2, and VH CDR3 of a TRBC1 antibody provided herein. In some embodiments, second binding domain that binds TRBC1 has a VL CDR1, VL CDR2, and VL CDR3 of a TRBC1 antibody provided herein. In some embodiments, second binding domain that binds TRBC1 has a VH CDR1, VH CDR2, VH CDR3, VL CDR1, VL CDR2, and VL CDR3 of a TRBC1 antibody provided herein.

In some embodiments, the second target is BCMA. In some embodiments, second binding domain that binds BCMA has a VH region, VL region, VH CDR1, VH CDR2, VH CDR3, VL CDR1, V CDR2, and/or VL CDR3 of a BCMA antibody provided herein. In some embodiments, second binding domain that binds BCMA has a VH region of a BCMA antibody provided herein. In some embodiments, second binding domain that binds BCMA has a VL region of a BCMA antibody provided herein. In some embodiments, second binding domain that binds BCMA has a VH region and a VL region of a BCMA antibody provided herein. In some embodiments, second binding domain that binds BCMA has a VH CDR1, VH CDR2, and VH CDR3 of a BCMA antibody provided herein. In some embodiments, second binding domain that binds BCMA has a VL CDR1, VL CDR2, and VL CDR3 of a BCMA antibody provided herein. In some embodiments, second binding domain that binds BCMA has a VH CDR1, VH CDR2, VH CDR3, VL CDR1, VL CDR2, and VL CDR3 of a BCMA antibody provided herein.

In some embodiments, the second target is PSMA. In some embodiments, second binding domain that binds PSMA has a VH region, VL region, VH CDR1, VH CDR2, VH CDR3, VL CDR1, VL CDR2, and/or VL CDR3 of a PSMA antibody provided herein. In some embodiments, second binding domain that binds PSMA has a VH region of a PSMA antibody provided herein. In some embodiments, second binding domain that binds PSMA has a VL region of a PSMA antibody provided herein. In some embodiments, second binding domain that binds PSMA has a VH region and a VL region of a PSMA antibody provided herein. In some embodiments, second binding domain that binds PSMA has a VH CDR1, VH CDR2, and VH CDR3 of a PSMA antibody provided herein. In some embodiments, second binding domain that binds PSMA has a VL CDR1, VL CDR2, and VL CDR3 of a PSMA antibody provided herein. In some embodiments, second binding domain that binds PSMA has a VH CDR1, VH CDR2, VH CDR3, VL CDR1, VL CDR2, and VL CDR3 of a PSMA antibody provided herein.

In some embodiments, the antibody specifically binds TRGV9. In other embodiments, the TRGV9 is present on the surface of a T cell.

In some embodiments, the TRGV9 antibody is chimeric. In some embodiments, the TRGV9 antibody is human. In some embodiments, the TRGV9 antibody is humanized. In certain embodiments, the TRGV9 antibody is an isolated TRGV9 antibody. In certain embodiments, provided is a TRGV9 antibody that is an intact antibody.

In some embodiments, the TRGV9 antibody is an IgG antibody. In some embodiments, the TRGV9 antibody is an IgG1 antibody. In some embodiments, the TRGV9 antibody is an IgG2 antibody. In some embodiments, the TRGV9 antibody is an IgG3 antibody. In some embodiments, the TRGV9 antibody is an IgG4 antibody. In some embodiments, the TRGV9 antibody comprises a kappa light chain. In some embodiments, the TRGV9 antibody comprises a lambda light chain. In some embodiments, the TRGV9 antibody is a monoclonal antibody. In some embodiments, the TRGV9 antibody is multivalent. In some embodiments, the TRGV9 antibody is capable of binding at least three antigens. In some embodiments, the TRGV9 antibody is capable of binding at least four antigens. In some embodiments, the TRGV9 antibody is capable of binding at least five antigens. In some embodiments, the TRGV9 antibody is a multispecific antibody. In some embodiments, the TRGV9 antibody is a bispecific antibody. In some embodiments, the TRGV9 antibody is a trispecific antibody. In some embodiments, the TRGV9 antibody is a quadraspecific antibody.

In other embodiments, provided is a TRGV9 antibody is an antigen binding fragment of the TRGV9 antibody. In some embodiments, the antigen binding fragment of the TRGV9 antibody is a functional fragment. In some embodiments, the TRGV9 antigen binding fragment is chimeric. In some embodiments, the TRGV9 antigen binding fragment is human. In some embodiments, a TRGV9 antigen binding fragment is humanized. In certain embodiments, a TRGV9 antigen binding fragment is an isolated TRGV9 antigen binding fragment.

In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)₂. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a bispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a camelized single domain antibody. In some embodiments, the antigen binding fragment is a nanobody. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure.

In some embodiments, the TRGV9 antibody is a multispecific antibody. In other embodiments, the TRGV9 antibody is a bispecific antibody. In certain embodiments, the multispecific antibody comprises an antigen binding fragment of a TRGV9 antibody provided herein. In other embodiments, the bispecific antibody comprises an antigen binding fragment of a TRGV9 antibody provided herein. In some embodiments, the TRGV9 antibody is an agonistic antibody. In certain embodiments, the TRGV9 antibody activates T cells. In other embodiments, the TRGV9 antibody is an antagonistic antibody. In certain embodiments, the TRGV9 antibody inactivates T cells. In some embodiments, the TRGV9 antibody blocks activation of T cells. In some embodiments, the TRGV9 antibody modulates the activity of T cells. In some embodiments, the TRGV9 antibody neither activates or inactivates the activity of γδ T cells. In a specific embodiment, the T cells are γδ T cells.

In specific embodiments, the γδ T cells are human γδ T cells. In specific embodiments, provided is a bispecific antibody comprising a TRGV9 antibody provided herein in a knob-in-hole format. In some embodiments, a TRGV9 antibody provided herein may be comprised in a bispecific antibody. In some embodiments, a TRGV9 bispecific antibody provided herein may be comprised in a multispecific antibody. In certain embodiments, a bispecific antibody provided herein comprises a first binding domain comprising a TRGV9 antibody provided herein that binds to a first TRGV9 epitope, and a second binding domain comprising a TRGV9 antibody provided herein that binds to a second TRGV9 epitope, wherein the first TRGV9 epitope and the second TRGV9 epitope are not the same. In a specific embodiment, a TRGV9 antibody, or antigen binding fragment thereof, provided herein specifically binds to TRGV9. In certain embodiments, a TRGV9 antibody, or antigen binding fragment thereof, provided herein does not bind to an epitope of Vδ2.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In one aspect, a TRGV9 antibody provided herein has a VH and VL amino acid sequence of L7A5_1 (TRGV9_1). In one aspect, provided herein is a TRGV9 antibody, wherein the antibody comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:7. In one aspect, provided herein is a TRGV9 antibody, wherein the antibody comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In one aspect, provided herein is a TRGV9 antibody, wherein the antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:7; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In another aspect, provided herein is a TRGV9 antibody, wherein the antibody comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is a TRGV9 antibody, wherein the antibody comprises: (i) a VH comprising a VH CDR1 having an amino acid sequence of SEQ ID NO:160, a VH CDR2 having an amino acid sequence of SEQ ID NO:161, and a VH CDR3 having an amino acid sequence of SEQ ID NO:162; and (ii) a VL comprising a VL CDR1 having an amino acid sequence of SEQ ID NO:163, a VL CDR2 having an amino acid sequence of SEQ ID NO:164, and a VL CDR3 having an amino acid sequence of SEQ ID NO:165. In another aspect, provided herein is a TRGV9 antibody, wherein the antibody comprises: (i) a VH comprising a VH CDR1 having an amino acid sequence of SEQ ID NO:166, a VH CDR2 having an amino acid sequence of SEQ ID NO:167, and a VH CDR3 having an amino acid sequence of SEQ ID NO:168; and (ii) a VL comprising a VL CDR1 having an amino acid sequence of SEQ ID NO:169, a VL CDR2 having an amino acid sequence of SEQ ID NO:170, and a VL CDR3 having an amino acid sequence of SEQ ID NO:171. In another aspect, provided herein is a TRGV9 antibody, wherein the antibody comprises: (i) a VH comprising a VH CDR1 having an amino acid sequence of SEQ ID NO:172, a VH CDR2 having an amino acid sequence of SEQ ID NO:173, and a VH CDR3 having an amino acid sequence of SEQ ID NO:174; and (ii) a VL comprising a VL CDR1 having an amino acid sequence of SEQ ID NO:175, a VL CDR2 having an amino acid sequence of SEQ ID NO:176, and a VL CDR3 having an amino acid sequence of SEQ ID NO:177. In another aspect, provided herein is a TRGV9 antibody, wherein the antibody comprises: (i) a VH comprising a VH CDR1 having an amino acid sequence of SEQ ID NO:178, a VH CDR2 having an amino acid sequence of SEQ ID NO:179, and a VH CDR3 having an amino acid sequence of SEQ ID NO:180; and (ii) a VL comprising a VL CDR1 having an amino acid sequence of SEQ ID NO:181, a VL CDR2 having an amino acid sequence of SEQ ID NO:182, and a VL CDR3 having an amino acid sequence of SEQ ID NO:183. In another aspect, provided herein is a TRGV9 antibody, wherein the antibody comprises: (i) a VH comprising a VH CDR1 having an amino acid sequence of SEQ ID NO:178, a VH CDR2 having an amino acid sequence of SEQ ID NO:700, and a VH CDR3 having an amino acid sequence of SEQ ID NO:701; and (ii) a VL comprising a VL CDR1 having an amino acid sequence of SEQ ID NO:181, a VL CDR2 having an amino acid sequence of SEQ ID NO:182, and a VL CDR3 having an amino acid sequence of SEQ ID NO:183. In another aspect, provided herein is a TRGV9 antibody, wherein the antibody comprises: (i) a VH comprising a VH CDR1 having an amino acid sequence of SEQ ID NO:184, a VH CDR2 having an amino acid sequence of SEQ ID NO:185, and a VH CDR3 having an amino acid sequence of SEQ ID NO:186; and (ii) a VL comprising a VL CDR1 having an amino acid sequence of SEQ ID NO:187, a VL CDR2 having an amino acid sequence of SEQ ID NO:188, and a VL CDR3 having an amino acid sequence of SEQ ID NO:189. In another aspect, provided herein is a TRGV9 antibody, wherein the antibody comprises: (i) a VH comprising a VH CDR1 having an amino acid sequence of SEQ ID NO:190, a VH CDR2 having an amino acid sequence of SEQ ID NO:191, and a VH CDR3 having an amino acid sequence of SEQ ID NO:192; and (ii) a VL comprising a VL CDR1 having an amino acid sequence of SEQ ID NO:193, a VL CDR2 having an amino acid sequence of SEQ ID NO:194, and a VL CDR3 having an amino acid sequence of SEQ ID NO:195. In some embodiments, the antibody comprises a VH having an amino acid sequence of SEQ ID NO:7. In some embodiments, the antibody comprises a VL having an amino acid sequence of SEQ ID NO:8. In some embodiments, the antibody comprises a VH having an amino acid sequence of SEQ ID NO:7, and a VL having an amino acid sequence of SEQ ID NO:8. In some embodiments, the antibody comprises a heavy chain having an amino acid sequence of SEQ ID NO:23. In some embodiments, the antibody comprises a light chain having an amino acid sequence of SEQ ID NO:24. In some embodiments, the antibody comprises a heavy chain having an amino acid sequence of SEQ ID NO:23, and a light chain having an amino acid sequence of SEQ ID NO:24. In some embodiments, the antibody comprises an amino acid sequence of SEQ ID NO:17. In some embodiments, the antibody comprises a heavy chain having an amino acid sequence of SEQ ID NO:69. In some embodiments, the antibody comprises a light chain having an amino acid sequence of SEQ ID NO:24. In some embodiments, the antibody comprises a heavy chain having an amino acid sequence of SEQ ID NO:69, and a light chain having an amino acid sequence of SEQ ID NO:24. In some embodiments, the antibody comprises a VH comprising an amino acid sequence having at least 95% identity to the amino acid sequence of SEQ ID NO:7. In some embodiments, the antibody comprises a VL comprising an amino acid sequence having at least 95% identity to the amino acid sequence of SEQ ID NO:8. In some embodiments, the antibody comprises a VH comprising an amino acid sequence having at least 95% identity to the amino acid sequence of SEQ ID NO:7, and a VL comprising an amino acid sequence having at least 95% identity to the amino acid sequence of SEQ ID NO:8. In some embodiments, the antibody comprises a heavy chain comprising an amino acid sequence having at least 95% identity to the amino acid sequence of SEQ ID NO:23. In some embodiments, the antibody comprises a light chain comprising an amino acid sequence having at least 95% identity to the amino acid sequence of SEQ ID NO:24. In some embodiments, the antibody comprises a heavy chain comprising an amino acid sequence having at least 95% identity to the amino acid sequence of SEQ ID NO:23, and a light chain comprising an amino acid sequence having at least 95% identity to the amino acid sequence of SEQ ID NO:24. In some embodiments, the antibody comprises an amino acid sequence of SEQ ID NO:17. In some embodiments, the antibody comprises a heavy chain comprising an amino acid sequence having at least 95% identity to the amino acid sequence of SEQ ID NO:69. In some embodiments, the antibody comprises a light chain comprising an amino acid sequence having at least 95% identity to the amino acid sequence of SEQ ID NO:24. In some embodiments, the antibody comprises a heavy chain comprising an amino acid sequence having at least 95% identity to the amino acid sequence of SEQ ID NO:69, and a light chain comprising an amino acid sequence having at least 95% identity to the amino acid sequence of SEQ ID NO:24.

In one aspect, a TRGV9 antibody provided herein has a VH and VL amino acid sequence of TRGV9Ab_2 (L7A5_2). In one aspect, provided herein is a TRGV9 antibody, wherein the antibody comprises a VH comprising a VH CDR1, a VH CDR2, and a VH CDR3 having amino acid sequences of the VH CDR1, VH CDR2, and VH CDR3, respectively, of SEQ ID NO:34. In one aspect, provided herein is a TRGV9 antibody, wherein the antibody comprises a VL comprising a VL CDR1, a VL CDR2, and a VL CDR3 having amino acid sequences of the VL CDR1, VL CDR2, and VL CDR3, respectively, of SEQ ID NO:8. In one aspect, provided herein is a TRGV9 antibody, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:34; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a TRGV9 antibody, wherein the antibody comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:31; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is a TRGV9 antibody, wherein the antibody comprises: (i) a VH comprising a VH CDR1 having an amino acid sequence of SEQ ID NO:196, a VH CDR2 having an amino acid sequence of SEQ ID NO:197, and a VH CDR3 having an amino acid sequence of SEQ ID NO:198; and (ii) a VL comprising a VL CDR1 having an amino acid sequence of SEQ ID NO:199, a VL CDR2 having an amino acid sequence of SEQ ID NO:200, and a VL CDR3 having an amino acid sequence of SEQ ID NO:201. In another aspect, provided herein is a TRGV9 antibody, wherein the antibody comprises: (i) a VH comprising a VH CDR1 having an amino acid sequence of SEQ ID NO:202, a VH CDR2 having an amino acid sequence of SEQ ID NO:203, and a VH CDR3 having an amino acid sequence of SEQ ID NO:204; and (ii) a VL comprising a VL CDR1 having an amino acid sequence of SEQ ID NO:205, a VL CDR2 having an amino acid sequence of SEQ ID NO:206, and a VL CDR3 having an amino acid sequence of SEQ ID NO:207. In another aspect, provided herein is a TRGV9 antibody, wherein the antibody comprises: (i) a VH comprising a VH CDR1 having an amino acid sequence of SEQ ID NO:208, a VH CDR2 having an amino acid sequence of SEQ ID NO:209, and a VH CDR3 having an amino acid sequence of SEQ ID NO:210; and (ii) a VL comprising a VL CDR1 having an amino acid sequence of SEQ ID NO:211, a VL CDR2 having an amino acid sequence of SEQ ID NO:212, and a VL CDR3 having an amino acid sequence of SEQ ID NO:213. In another aspect, provided herein is a TRGV9 antibody, wherein the antibody comprises: (i) a VH comprising a VH CDR1 having an amino acid sequence of SEQ ID NO:214, a VH CDR2 having an amino acid sequence of SEQ ID NO:215, and a VH CDR3 having an amino acid sequence of SEQ ID NO:216; and (ii) a VL comprising a VL CDR1 having an amino acid sequence of SEQ ID NO:217, a VL CDR2 having an amino acid sequence of SEQ ID NO:218, and a VL CDR3 having an amino acid sequence of SEQ ID NO:219. In another aspect, provided herein is a TRGV9 antibody, wherein the antibody comprises: (i) a VH comprising a VH CDR1 having an amino acid sequence of SEQ ID NO:214, a VH CDR2 having an amino acid sequence of SEQ ID NO:702, and a VH CDR3 having an amino acid sequence of SEQ ID NO:703; and (ii) a VL comprising a VL CDR1 having an amino acid sequence of SEQ ID NO:217, a VL CDR2 having an amino acid sequence of SEQ ID NO:218, and a VL CDR3 having an amino acid sequence of SEQ ID NO:219. In another aspect, provided herein is a TRGV9 antibody, wherein the antibody comprises: (i) a VH comprising a VH CDR1 having an amino acid sequence of SEQ ID NO:220, a VH CDR2 having an amino acid sequence of SEQ ID NO:221, and a VH CDR3 having an amino acid sequence of SEQ ID NO:222; and (ii) a VL comprising a VL CDR1 having an amino acid sequence of SEQ ID NO:223, a VL CDR2 having an amino acid sequence of SEQ ID NO:224, and a VL CDR3 having an amino acid sequence of SEQ ID NO:225. In another aspect, provided herein is a TRGV9 antibody, wherein the antibody comprises: (i) a VH comprising a VH CDR1 having an amino acid sequence of SEQ ID NO:226, a VH CDR2 having an amino acid sequence of SEQ ID NO:227, and a VH CDR3 having an amino acid sequence of SEQ ID NO:228; and (ii) a VL comprising a VL CDR1 having an amino acid sequence of SEQ ID NO:229, a VL CDR2 having an amino acid sequence of SEQ ID NO:230, and a VL CDR3 having an amino acid sequence of SEQ ID NO:231. In some embodiments, the antibody comprises a VH having an amino acid sequence of SEQ ID NO:34. In some embodiments, the antibody comprises a VL having an amino acid sequence of SEQ ID NO:8. In some embodiments, the antibody comprises a VH having an amino acid sequence of SEQ ID NO:34, and a VL having an amino acid sequence of SEQ ID NO:8. In some embodiments, the antibody comprises a VH comprising an amino acid sequence having at least 95% identity to the amino acid sequence of SEQ ID NO:34. In some embodiments, the antibody comprises a VL comprising an amino acid sequence having at least 95% identity to the amino acid sequence of SEQ ID NO:8. In some embodiments, the antibody comprises a VH comprising an amino acid sequence having at least 95% identity to the amino acid sequence of SEQ ID NO:34, and a VL comprising an amino acid sequence having at least 95% identity to the amino acid sequence of SEQ ID NO:8.

In one aspect, a TRGV9 antibody provided herein has a VH and VL amino acid sequence of TRGV9Ab_3 (L7A5_3). In one aspect, provided herein is a TRGV9 antibody, wherein the antibody comprises a VH comprising a VH CDR1, a VH CDR2, and a VH CDR3 having amino acid sequences of the VH CDR1, VH CDR2, and VH CDR3, respectively, of SEQ ID NO:35. In one aspect, provided herein is a TRGV9 antibody, wherein the antibody comprises a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a TRGV9 antibody, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:35; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a TRGV9 antibody, wherein the antibody comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:32; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is a TRGV9 antibody, wherein the antibody comprises: (i) a VH comprising a VH CDR1 having an amino acid sequence of SEQ ID NO:232, a VH CDR2 having an amino acid sequence of SEQ ID NO:233, and a VH CDR3 having an amino acid sequence of SEQ ID NO:234; and (ii) a VL comprising a VL CDR1 having an amino acid sequence of SEQ ID NO:235, a VL CDR2 having an amino acid sequence of SEQ ID NO:236, and a VL CDR3 having an amino acid sequence of SEQ ID NO:237. In another aspect, provided herein is a TRGV9 antibody, wherein the antibody comprises: (i) a VH comprising a VH CDR1 having an amino acid sequence of SEQ ID NO:238, a VH CDR2 having an amino acid sequence of SEQ ID NO:239, and a VH CDR3 having an amino acid sequence of SEQ ID NO:240; and (ii) a VL comprising a VL CDR1 having an amino acid sequence of SEQ ID NO:241, a VL CDR2 having an amino acid sequence of SEQ ID NO:242, and a VL CDR3 having an amino acid sequence of SEQ ID NO:243. In another aspect, provided herein is a TRGV9 antibody, wherein the antibody comprises: (i) a VH comprising a VH CDR1 having an amino acid sequence of SEQ ID NO:244, a VH CDR2 having an amino acid sequence of SEQ ID NO:245, and a VH CDR3 having an amino acid sequence of SEQ ID NO:246; and (ii) a VL comprising a VL CDR1 having an amino acid sequence of SEQ ID NO:247, a VL CDR2 having an amino acid sequence of SEQ ID NO:248, and a VL CDR3 having an amino acid sequence of SEQ ID NO:249. In another aspect, provided herein is a TRGV9 antibody, wherein the antibody comprises: (i) a VH comprising a VH CDR1 having an amino acid sequence of SEQ ID NO:250, a VH CDR2 having an amino acid sequence of SEQ ID NO:251, and a VH CDR3 having an amino acid sequence of SEQ ID NO:252; and (ii) a VL comprising a VL CDR1 having an amino acid sequence of SEQ ID NO:253, a VL CDR2 having an amino acid sequence of SEQ ID NO:254, and a VL CDR3 having an amino acid sequence of SEQ ID NO:255. In another aspect, provided herein is a TRGV9 antibody, wherein the antibody comprises: (i) a VH comprising a VH CDR1 having an amino acid sequence of SEQ ID NO:250, a VH CDR2 having an amino acid sequence of SEQ ID NO:704, and a VH CDR3 having an amino acid sequence of SEQ ID NO:705; and (ii) a VL comprising a VL CDR1 having an amino acid sequence of SEQ ID NO:253, a VL CDR2 having an amino acid sequence of SEQ ID NO:254, and a VL CDR3 having an amino acid sequence of SEQ ID NO:255. In another aspect, provided herein is a TRGV9 antibody, wherein the antibody comprises: (i) a VH comprising a VH CDR1 having an amino acid sequence of SEQ ID NO:256, a VH CDR2 having an amino acid sequence of SEQ ID NO:257, and a VH CDR3 having an amino acid sequence of SEQ ID NO:258; and (ii) a VL comprising a VL CDR1 having an amino acid sequence of SEQ ID NO:259, a VL CDR2 having an amino acid sequence of SEQ ID NO:260, and a VL CDR3 having an amino acid sequence of SEQ ID NO:261. In another aspect, provided herein is a TRGV9 antibody, wherein the antibody comprises: (i) a VH comprising a VH CDR1 having an amino acid sequence of SEQ ID NO:262, a VH CDR2 having an amino acid sequence of SEQ ID NO:263, and a VH CDR3 having an amino acid sequence of SEQ ID NO:264; and (ii) a VL comprising a VL CDR1 having an amino acid sequence of SEQ ID NO:265, a VL CDR2 having an amino acid sequence of SEQ ID NO:266, and a VL CDR3 having an amino acid sequence of SEQ ID NO:267. In some embodiments, the antibody comprises a VH having an amino acid sequence of SEQ ID NO:35. In some embodiments, the antibody comprises a VL having an amino acid sequence of SEQ ID NO:8. In some embodiments, the antibody comprises a VH having an amino acid sequence of SEQ ID NO:35, and a VL having an amino acid sequence of SEQ ID NO:8. In some embodiments, the antibody comprises a VH comprising an amino acid sequence having at least 95% identity to the amino acid sequence of SEQ ID NO:35. In some embodiments, the antibody comprises a VL comprising an amino acid sequence having at least 95% identity to the amino acid sequence of SEQ ID NO:8. In some embodiments, the antibody comprises a VH comprising an amino acid sequence having at least 95% identity to the amino acid sequence of SEQ ID NO:35, and a VL comprising an amino acid sequence having at least 95% identity to the amino acid sequence of SEQ ID NO:8.

In one aspect, a TRGV9 antibody provided herein has a VH and VL amino acid sequence of TRGV9Ab_4 (L7A5_4). In one aspect, provided herein is a TRGV9 antibody, wherein the antibody comprises a VH comprising a VH CDR1, a VH CDR2, and a VH CDR3 having amino acid sequences of the VH CDR1, VH CDR2, and VH CDR3, respectively, of SEQ ID NO:36. In one aspect, provided herein is a TRGV9 antibody, wherein the antibody comprises a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a TRGV9 antibody, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:36; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a TRGV9 antibody, wherein the antibody comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:33; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is a TRGV9 antibody, wherein the antibody comprises: (i) a VH comprising a VH CDR1 having an amino acid sequence of SEQ ID NO:268, a VH CDR2 having an amino acid sequence of SEQ ID NO:269, and a VH CDR3 having an amino acid sequence of SEQ ID NO:270; and (ii) a VL comprising a VL CDR1 having an amino acid sequence of SEQ ID NO:271, a VL CDR2 having an amino acid sequence of SEQ ID NO:272, and a VL CDR3 having an amino acid sequence of SEQ ID NO:273. In another aspect, provided herein is a TRGV9 antibody, wherein the antibody comprises: (i) a VH comprising a VH CDR1 having an amino acid sequence of SEQ ID NO:274, a VH CDR2 having an amino acid sequence of SEQ ID NO:275, and a VH CDR3 having an amino acid sequence of SEQ ID NO:276; and (ii) a VL comprising a VL CDR1 having an amino acid sequence of SEQ ID NO:277, a VL CDR2 having an amino acid sequence of SEQ ID NO:278, and a VL CDR3 having an amino acid sequence of SEQ ID NO:279. In another aspect, provided herein is a TRGV9 antibody, wherein the antibody comprises: (i) a VH comprising a VH CDR1 having an amino acid sequence of SEQ ID NO:280, a VH CDR2 having an amino acid sequence of SEQ ID NO:281, and a VH CDR3 having an amino acid sequence of SEQ ID NO:282; and (ii) a VL comprising a VL CDR1 having an amino acid sequence of SEQ ID NO:283, a VL CDR2 having an amino acid sequence of SEQ ID NO:284, and a VL CDR3 having an amino acid sequence of SEQ ID NO:285. In another aspect, provided herein is a TRGV9 antibody, wherein the antibody comprises: (i) a VH comprising a VH CDR1 having an amino acid sequence of SEQ ID NO:286, a VH CDR2 having an amino acid sequence of SEQ ID NO:287, and a VH CDR3 having an amino acid sequence of SEQ ID NO:288; and (ii) a VL comprising a VL CDR1 having an amino acid sequence of SEQ ID NO:289, a VL CDR2 having an amino acid sequence of SEQ ID NO:290, and a VL CDR3 having an amino acid sequence of SEQ ID NO:291. In another aspect, provided herein is a TRGV9 antibody, wherein the antibody comprises: (i) a VH comprising a VH CDR1 having an amino acid sequence of SEQ ID NO:286, a VH CDR2 having an amino acid sequence of SEQ ID NO:706, and a VH CDR3 having an amino acid sequence of SEQ ID NO:707; and (ii) a VL comprising a VL CDR1 having an amino acid sequence of SEQ ID NO:289, a VL CDR2 having an amino acid sequence of SEQ ID NO:290, and a VL CDR3 having an amino acid sequence of SEQ ID NO:291. In another aspect, provided herein is a TRGV9 antibody, wherein the antibody comprises: (i) a VH comprising a VH CDR1 having an amino acid sequence of SEQ ID NO:292, a VH CDR2 having an amino acid sequence of SEQ ID NO:293, and a VH CDR3 having an amino acid sequence of SEQ ID NO:294; and (ii) a VL comprising a VL CDR1 having an amino acid sequence of SEQ ID NO:295, a VL CDR2 having an amino acid sequence of SEQ ID NO:296, and a VL CDR3 having an amino acid sequence of SEQ ID NO:297. In another aspect, provided herein is a TRGV9 antibody, wherein the antibody comprises: (i) a VH comprising a VH CDR1 having an amino acid sequence of SEQ ID NO:298, a VH CDR2 having an amino acid sequence of SEQ ID NO:299, and a VH CDR3 having an amino acid sequence of SEQ ID NO:300; and (ii) a VL comprising a VL CDR1 having an amino acid sequence of SEQ ID NO:301, a VL CDR2 having an amino acid sequence of SEQ ID NO:302, and a VL CDR3 having an amino acid sequence of SEQ ID NO:303. In some embodiments, the antibody comprises a VH having an amino acid sequence of SEQ ID NO:36. In some embodiments, the antibody comprises a VL having an amino acid sequence of SEQ ID NO:8. In some embodiments, the antibody comprises a VH having an amino acid sequence of SEQ ID NO:36, and a VL having an amino acid sequence of SEQ ID NO:8. In some embodiments, the antibody comprises a VH comprising an amino acid sequence having at least 95% identity to the amino acid sequence of SEQ ID NO:36. In some embodiments, the antibody comprises a VL comprising an amino acid sequence having at least 95% identity to the amino acid sequence of SEQ ID NO:8. In some embodiments, the antibody comprises a VH comprising an amino acid sequence having at least 95% identity to the amino acid sequence of SEQ ID NO:36, and a VL comprising an amino acid sequence having at least 95% identity to the amino acid sequence of SEQ ID NO:8.

In one aspect, a TRGV9 antibody provided herein has a VH and VL amino acid sequence of TRGV9Ab_var17. In one aspect, a TRGV9 antibody provided herein has a VH and VL amino acid sequence of TRGV9Ab_var29. In one aspect, provided herein is a TRGV9 antibody, wherein the antibody comprises a VH comprising a VH CDR1, a VH CDR2, and a VH CDR3 having amino acid sequences of the VH CDR1, VH CDR2, and VH CDR3, respectively, of SEQ ID NO:65. In one aspect, provided herein is a TRGV9 antibody, wherein the antibody comprises a VL comprising a VL CDR1, a VL CDR2, and a VL CDR3 having amino acid sequences of the VL CDR1, VL CDR2, and VL CDR3, respectively, of SEQ ID NO:66. In another aspect, provided herein is a TRGV9 antibody, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:65; and (ii) a VL comprising a VL CDR1, a VL CDR2, and a VL CDR3 having amino acid sequences of the VL CDR1, VL CDR2, and VL CDR3, respectively, of SEQ ID NO:66. In another aspect, provided herein is a TRGV9 antibody, wherein the antibody comprises a VH comprising a VH CDR1, a VH CDR2, and a VH CDR3 having amino acid sequences of the VH CDR1, VH CDR2, and VH CDR3, respectively, of SEQ ID NO:67. In another aspect, provided herein is a TRGV9 antibody, wherein the antibody comprises a VL comprising a VL CDR1, a VL CDR2, and a VL CDR3 having amino acid sequences of the VL CDR1, VL CDR2, and VL CDR3, respectively, of SEQ ID NO:68. In another aspect, provided herein is a TRGV9 antibody, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:67; and (ii) a VL comprising a VL CDR1, a VL CDR2, and a VL CDR3 having amino acid sequences of the VL CDR1, VL CDR2, and VL CDR3, respectively, of SEQ ID NO:68. In another aspect, provided herein is a TRGV9 antibody, wherein the antibody comprises: (i) a VH comprising a VH CDR1 having an amino acid sequence of SEQ ID NO:1, a VH CDR2 having an amino acid sequence of SEQ ID NO:76, and a VH CDR3 having an amino acid sequence of SEQ ID NO:3; and (ii) a VL comprising a VL CDR1 having an amino acid sequence of SEQ ID NO:77, a VL CDR2 having an amino acid sequence of SEQ ID NO:5, and a VL CDR3 having an amino acid sequence of SEQ ID NO:6. In another aspect, provided herein is a TRGV9 antibody, wherein the antibody comprises: (i) a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and (ii) a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6. In another aspect, provided herein is a TRGV9 antibody, wherein the antibody comprises: (i) a VH comprising a VH CDR1 having an amino acid sequence of SEQ ID NO:304, a VH CDR2 having an amino acid sequence of SEQ ID NO:305, and a VH CDR3 having an amino acid sequence of SEQ ID NO:306; and (ii) a VL comprising a VL CDR1 having an amino acid sequence of SEQ ID NO:307, a VL CDR2 having an amino acid sequence of SEQ ID NO:308, and a VL CDR3 having an amino acid sequence of SEQ ID NO:309. In another aspect, provided herein is a TRGV9 antibody, wherein the antibody comprises: (i) a VH comprising a VH CDR1 having an amino acid sequence of SEQ ID NO:310, a VH CDR2 having an amino acid sequence of SEQ ID NO:311, and a VH CDR3 having an amino acid sequence of SEQ ID NO:312; and (ii) a VL comprising a VL CDR1 having an amino acid sequence of SEQ ID NO:313, a VL CDR2 having an amino acid sequence of SEQ ID NO:314, and a VL CDR3 having an amino acid sequence of SEQ ID NO:315. In another aspect, provided herein is a TRGV9 antibody, wherein the antibody comprises: (i) a VH comprising a VH CDR1 having an amino acid sequence of SEQ ID NO:316, a VH CDR2 having an amino acid sequence of SEQ ID NO:317, and a VH CDR3 having an amino acid sequence of SEQ ID NO:318; and (ii) a VL comprising a VL CDR1 having an amino acid sequence of SEQ ID NO:319, a VL CDR2 having an amino acid sequence of SEQ ID NO:320, and a VL CDR3 having an amino acid sequence of SEQ ID NO:321. In another aspect, provided herein is a TRGV9 antibody, wherein the antibody comprises: (i) a VH comprising a VH CDR1 having an amino acid sequence of SEQ ID NO:322, a VH CDR2 having an amino acid sequence of SEQ ID NO:323, and a VH CDR3 having an amino acid sequence of SEQ ID NO:324; and (ii) a VL comprising a VL CDR1 having an amino acid sequence of SEQ ID NO:325, a VL CDR2 having an amino acid sequence of SEQ ID NO:326, and a VL CDR3 having an amino acid sequence of SEQ ID NO:327. In another aspect, provided herein is a TRGV9 antibody, wherein the antibody comprises: (i) a VH comprising a VH CDR1 having an amino acid sequence of SEQ ID NO:322, a VH CDR2 having an amino acid sequence of SEQ ID NO:708, and a VH CDR3 having an amino acid sequence of SEQ ID NO:709; and (ii) a VL comprising a VL CDR1 having an amino acid sequence of SEQ ID NO:325, a VL CDR2 having an amino acid sequence of SEQ ID NO:326, and a VL CDR3 having an amino acid sequence of SEQ ID NO:327. In another aspect, provided herein is a TRGV9 antibody, wherein the antibody comprises: (i) a VH comprising a VH CDR1 having an amino acid sequence of SEQ ID NO:328, a VH CDR2 having an amino acid sequence of SEQ ID NO:329, and a VH CDR3 having an amino acid sequence of SEQ ID NO:330; and (ii) a VL comprising a VL CDR1 having an amino acid sequence of SEQ ID NO:331, a VL CDR2 having an amino acid sequence of SEQ ID NO:332, and a VL CDR3 having an amino acid sequence of SEQ ID NO:333. In another aspect, provided herein is a TRGV9 antibody, wherein the antibody comprises: (i) a VH comprising a VH CDR1 having an amino acid sequence of SEQ ID NO:334, a VH CDR2 having an amino acid sequence of SEQ ID NO:335, and a VH CDR3 having an amino acid sequence of SEQ ID NO:336; and (ii) a VL comprising a VL CDR1 having an amino acid sequence of SEQ ID NO:337, a VL CDR2 having an amino acid sequence of SEQ ID NO:338, and a VL CDR3 having an amino acid sequence of SEQ ID NO:339. In another aspect, provided herein is a TRGV9 antibody, wherein the antibody comprises: (i) a VH comprising a VH CDR1 having an amino acid sequence of SEQ ID NO:340, a VH CDR2 having an amino acid sequence of SEQ ID NO:341, and a VH CDR3 having an amino acid sequence of SEQ ID NO:342; and (ii) a VL comprising a VL CDR1 having an amino acid sequence of SEQ ID NO:343, a VL CDR2 having an amino acid sequence of SEQ ID NO:344, and a VL CDR3 having an amino acid sequence of SEQ ID NO:345. In another aspect, provided herein is a TRGV9 antibody, wherein the antibody comprises: (i) a VH comprising a VH CDR1 having an amino acid sequence of SEQ ID NO:346, a VH CDR2 having an amino acid sequence of SEQ ID NO:347, and a VH CDR3 having an amino acid sequence of SEQ ID NO:348; and (ii) a VL comprising a VL CDR1 having an amino acid sequence of SEQ ID NO:349, a VL CDR2 having an amino acid sequence of SEQ ID NO:350, and a VL CDR3 having an amino acid sequence of SEQ ID NO:351. In another aspect, provided herein is a TRGV9 antibody, wherein the antibody comprises: (i) a VH comprising a VH CDR1 having an amino acid sequence of SEQ ID NO:352, a VH CDR2 having an amino acid sequence of SEQ ID NO:353, and a VH CDR3 having an amino acid sequence of SEQ ID NO:354; and (ii) a VL comprising a VL CDR1 having an amino acid sequence of SEQ ID NO:355, a VL CDR2 having an amino acid sequence of SEQ ID NO:356, and a VL CDR3 having an amino acid sequence of SEQ ID NO:357. In another aspect, provided herein is a TRGV9 antibody, wherein the antibody comprises: (i) a VH comprising a VH CDR1 having an amino acid sequence of SEQ ID NO:358, a VH CDR2 having an amino acid sequence of SEQ ID NO:359, and a VH CDR3 having an amino acid sequence of SEQ ID NO:360; and (ii) a VL comprising a VL CDR1 having an amino acid sequence of SEQ ID NO:361, a VL CDR2 having an amino acid sequence of SEQ ID NO:362, and a VL CDR3 having an amino acid sequence of SEQ ID NO:363. In another aspect, provided herein is a TRGV9 antibody, wherein the antibody comprises: (i) a VH comprising a VH CDR1 having an amino acid sequence of SEQ ID NO:358, a VH CDR2 having an amino acid sequence of SEQ ID NO:710, and a VH CDR3 having an amino acid sequence of SEQ ID NO:711; and (ii) a VL comprising a VL CDR1 having an amino acid sequence of SEQ ID NO:361, a VL CDR2 having an amino acid sequence of SEQ ID NO:362, and a VL CDR3 having an amino acid sequence of SEQ ID NO:363. In another aspect, provided herein is a TRGV9 antibody, wherein the antibody comprises: (i) a VH comprising a VH CDR1 having an amino acid sequence of SEQ ID NO:364, a VH CDR2 having an amino acid sequence of SEQ ID NO:365, and a VH CDR3 having an amino acid sequence of SEQ ID NO:366; and (ii) a VL comprising a VL CDR1 having an amino acid sequence of SEQ ID NO:367, a VL CDR2 having an amino acid sequence of SEQ ID NO:368, and a VL CDR3 having an amino acid sequence of SEQ ID NO:369. In another aspect, provided herein is a TRGV9 antibody, wherein the antibody comprises: (i) a VH comprising a VH CDR1 having an amino acid sequence of SEQ ID NO:370, a VH CDR2 having an amino acid sequence of SEQ ID NO:371, and a VH CDR3 having an amino acid sequence of SEQ ID NO:372; and (ii) a VL comprising a VL CDR1 having an amino acid sequence of SEQ ID NO:373, a VL CDR2 having an amino acid sequence of SEQ ID NO:374, and a VL CDR3 having an amino acid sequence of SEQ ID NO:375. In some embodiments, the antibody comprises a VH having an amino acid sequence of SEQ ID NO:65. In some embodiments, the antibody comprises a VL having an amino acid sequence of SEQ ID NO:66. In some embodiments, the antibody comprises a VH having an amino acid sequence of SEQ ID NO:65, and a VL having an amino acid sequence of SEQ ID NO:66. In some embodiments, the antibody comprises a VH having an amino acid sequence of SEQ ID NO:67. In some embodiments, the antibody comprises a VL having an amino acid sequence of SEQ ID NO:68. In some embodiments, the antibody comprises a VH having an amino acid sequence of SEQ ID NO:67, and a VL having an amino acid sequence of SEQ ID NO:68. In some embodiments, the antibody comprises a heavy chain having an amino acid sequence of SEQ ID NO:71. In some embodiments, the antibody comprises a light chain having an amino acid sequence of SEQ ID NO:72. In some embodiments, the antibody comprises a heavy chain having an amino acid sequence of SEQ ID NO:71, and a light chain having an amino acid sequence of SEQ ID NO:72. In some embodiments, the antibody comprises an amino acid sequence of SEQ ID NO:70. In some embodiments, the antibody comprises a heavy chain having an amino acid sequence of SEQ ID NO:74. In some embodiments, the antibody comprises a light chain having an amino acid sequence of SEQ ID NO:75. In some embodiments, the antibody comprises a heavy chain having an amino acid sequence of SEQ ID NO:74, and a light chain having an amino acid sequence of SEQ ID NO:75. In some embodiments, the antibody comprises an amino acid sequence of SEQ ID NO:73. In some embodiments, the antibody comprises a VH comprising an amino acid sequence having at least 95% identity to the amino acid sequence of SEQ ID NO:65. In some embodiments, the antibody comprises a VL comprising an amino acid sequence having at least 95% identity to the amino acid sequence of SEQ ID NO:66. In some embodiments, the antibody comprises a VH comprising an amino acid sequence having at least 95% identity to the amino acid sequence of SEQ ID NO:65, and a VL comprising an amino acid sequence having at least 95% identity to the amino acid sequence of SEQ ID NO:66. In some embodiments, the antibody comprises a VH comprising an amino acid sequence having at least 95% identity to the amino acid sequence of SEQ ID NO:67. In some embodiments, the antibody comprises a VL comprising an amino acid sequence having at least 95% identity to the amino acid sequence of SEQ ID NO:68. In some embodiments, the antibody comprises a VH comprising an amino acid sequence having at least 95% identity to the amino acid sequence of SEQ ID NO:67, and a VL comprising an amino acid sequence having at least 95% identity to the amino acid sequence of SEQ ID NO:68. In some embodiments, the antibody comprises a heavy chain comprising an amino acid sequence having at least 95% identity to the amino acid sequence of SEQ ID NO:71. In some embodiments, the antibody comprises a light chain comprising an amino acid sequence having at least 95% identity to the amino acid sequence of SEQ ID NO:72. In some embodiments, the antibody comprises a heavy chain comprising an amino acid sequence having at least 95% identity to the amino acid sequence of SEQ ID NO:71, and a light chain comprising an amino acid sequence having at least 95% identity to the amino acid sequence of SEQ ID NO:72. In some embodiments, the antibody comprises an amino acid sequence of SEQ ID NO:70. In some embodiments, the antibody comprises a heavy chain comprising an amino acid sequence having at least 95% identity to the amino acid sequence of SEQ ID NO:74. In some embodiments, the antibody comprises a light chain comprising an amino acid sequence having at least 95% identity to the amino acid sequence of SEQ ID NO:75. In some embodiments, the antibody comprises a heavy chain comprising an amino acid sequence having at least 95% identity to the amino acid sequence of SEQ ID NO:74, and a light chain comprising an amino acid sequence having at least 95% identity to the amino acid sequence of SEQ ID NO:75. In some embodiments, the antibody comprises an amino acid sequence of SEQ ID NO:73.

In one aspect, a TRGV9 antibody provided herein has a VH and VL amino acid sequence of VG3_B3_RN. In one aspect, provided herein is a TRGV9 antibody, wherein the antibody comprises a VH comprising a VH CDR1, a VH CDR2, and a VH CDR3 having amino acid sequences of the VH CDR1, VH CDR2, and VH CDR3, respectively, of SEQ ID NO:95. In one aspect, provided herein is a TRGV9 antibody, wherein the antibody comprises a VL comprising a VL CDR1, a VL CDR2, and a VL CDR3 having amino acid sequences of the VL CDR1, VL CDR2, and VL CDR3, respectively, of SEQ ID NO:96. In another aspect, provided herein is a TRGV9 antibody, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:95; and (ii) a VL comprising a VL CDR1, a VL CDR2, and a VL CDR3 having amino acid sequences of the VL CDR1, VL CDR2, and VL CDR3, respectively, of SEQ ID NO:96. In another aspect, provided herein is a TRGV9 antibody, wherein the antibody comprises: (i) a VH comprising a VH CDR1 having an amino acid sequence of SEQ ID NO:89, a VH CDR2 having an amino acid sequence of SEQ ID NO:90, and a VH CDR3 having an amino acid sequence of SEQ ID NO:91; and (ii) a VL comprising a VL CDR1 having an amino acid sequence of SEQ ID NO:92, a VL CDR2 having an amino acid sequence of SEQ ID NO:93, and a VL CDR3 having an amino acid sequence of SEQ ID NO:94. In another aspect, provided herein is a TRGV9 antibody, wherein the antibody comprises: (i) a VH comprising a VH CDR1 having an amino acid sequence of SEQ ID NO:376, a VH CDR2 having an amino acid sequence of SEQ ID NO:377, and a VH CDR3 having an amino acid sequence of SEQ ID NO:378; and (ii) a VL comprising a VL CDR1 having an amino acid sequence of SEQ ID NO:379, a VL CDR2 having an amino acid sequence of SEQ ID NO:380, and a VL CDR3 having an amino acid sequence of SEQ ID NO:381. In another aspect, provided herein is a TRGV9 antibody, wherein the antibody comprises: (i) a VH comprising a VH CDR1 having an amino acid sequence of SEQ ID NO:382, a VH CDR2 having an amino acid sequence of SEQ ID NO:383, and a VH CDR3 having an amino acid sequence of SEQ ID NO:384; and (ii) a VL comprising a VL CDR1 having an amino acid sequence of SEQ ID NO:385, a VL CDR2 having an amino acid sequence of SEQ ID NO:386, and a VL CDR3 having an amino acid sequence of SEQ ID NO:387. In another aspect, provided herein is a TRGV9 antibody, wherein the antibody comprises: (i) a VH comprising a VH CDR1 having an amino acid sequence of SEQ ID NO:388, a VH CDR2 having an amino acid sequence of SEQ ID NO:389, and a VH CDR3 having an amino acid sequence of SEQ ID NO:390; and (ii) a VL comprising a VL CDR1 having an amino acid sequence of SEQ ID NO:391, a VL CDR2 having an amino acid sequence of SEQ ID NO:392, and a VL CDR3 having an amino acid sequence of SEQ ID NO:393. In another aspect, provided herein is a TRGV9 antibody, wherein the antibody comprises: (i) a VH comprising a VH CDR1 having an amino acid sequence of SEQ ID NO:394, a VH CDR2 having an amino acid sequence of SEQ ID NO:395, and a VH CDR3 having an amino acid sequence of SEQ ID NO:396; and (ii) a VL comprising a VL CDR1 having an amino acid sequence of SEQ ID NO:397, a VL CDR2 having an amino acid sequence of SEQ ID NO:398, and a VL CDR3 having an amino acid sequence of SEQ ID NO:399. In another aspect, provided herein is a TRGV9 antibody, wherein the antibody comprises: (i) a VH comprising a VH CDR1 having an amino acid sequence of SEQ ID NO:394, a VH CDR2 having an amino acid sequence of SEQ ID NO:712, and a VH CDR3 having an amino acid sequence of SEQ ID NO:713; and (ii) a VL comprising a VL CDR1 having an amino acid sequence of SEQ ID NO:397, a VL CDR2 having an amino acid sequence of SEQ ID NO:398, and a VL CDR3 having an amino acid sequence of SEQ ID NO:399. In another aspect, provided herein is a TRGV9 antibody, wherein the antibody comprises: (i) a VH comprising a VH CDR1 having an amino acid sequence of SEQ ID NO:400, a VH CDR2 having an amino acid sequence of SEQ ID NO:401, and a VH CDR3 having an amino acid sequence of SEQ ID NO:402; and (ii) a VL comprising a VL CDR1 having an amino acid sequence of SEQ ID NO:403, a VL CDR2 having an amino acid sequence of SEQ ID NO:404, and a VL CDR3 having an amino acid sequence of SEQ ID NO:405. In another aspect, provided herein is a TRGV9 antibody, wherein the antibody comprises: (i) a VH comprising a VH CDR1 having an amino acid sequence of SEQ ID NO:406, a VH CDR2 having an amino acid sequence of SEQ ID NO:407, and a VH CDR3 having an amino acid sequence of SEQ ID NO:408; and (ii) a VL comprising a VL CDR1 having an amino acid sequence of SEQ ID NO:409, a VL CDR2 having an amino acid sequence of SEQ ID NO:410, and a VL CDR3 having an amino acid sequence of SEQ ID NO:411. In some embodiments, the antibody comprises a VH having an amino acid sequence of SEQ ID NO:95. In some embodiments, the antibody comprises a VL having an amino acid sequence of SEQ ID NO:96. In some embodiments, the antibody comprises a VH having an amino acid sequence of SEQ ID NO:95, and a VL having an amino acid sequence of SEQ ID NO:96. In some embodiments, the antibody comprises an amino acid sequence of SEQ ID NO:97. In some embodiments, the antibody comprises a VH comprising an amino acid sequence having at least 95% identity to the amino acid sequence of SEQ ID NO:95. In some embodiments, the antibody comprises a VL comprising an amino acid sequence having at least 95% identity to the amino acid sequence of SEQ ID NO:96. In some embodiments, the antibody comprises a VH comprising an amino acid sequence having at least 95% identity to the amino acid sequence of SEQ ID NO:95, and a VL comprising an amino acid sequence having at least 95% identity to the amino acid sequence of SEQ ID NO:96. In some embodiments, the antibody comprises an amino acid sequence having at least 95% identity to an amino acid sequence of SEQ ID NO:97.

In one aspect, a TRGV9 antibody provided herein has a VH and VL amino acid sequence of VG9B420. In one aspect, provided herein is a TRGV9 antibody, wherein the antibody comprises a VH comprising a VH CDR1, a VH CDR2, and a VH CDR3 having amino acid sequences of the VH CDR1, VH CDR2, and VH CDR3, respectively, of SEQ ID NO:104. In one aspect, provided herein is a TRGV9 antibody, wherein the antibody comprises a VL comprising a VL CDR1, a VL CDR2, and a VL CDR3 having amino acid sequences of the VL CDR1, VL CDR2, and VL CDR3, respectively, of SEQ ID NO:105. In another aspect, provided herein is a TRGV9 antibody, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:104; and (ii) a VL comprising a VL CDR1, a VL CDR2, and a VL CDR3 having amino acid sequences of the VL CDR1, VL CDR2, and VL CDR3, respectively, of SEQ ID NO:105. In another aspect, provided herein is a TRGV9 antibody, wherein the antibody comprises: (i) a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100; and (ii) a VL comprising a VL CDR1 having an amino acid sequence of SEQ ID NO:101, a VL CDR2 having an amino acid sequence of SEQ ID NO:102, and a VL CDR3 having an amino acid sequence of SEQ ID NO:103. In another aspect, provided herein is a TRGV9 antibody, wherein the antibody comprises: (i) a VH comprising a VH CDR1 having an amino acid sequence of SEQ ID NO:412, a VH CDR2 having an amino acid sequence of SEQ ID NO:413, and a VH CDR3 having an amino acid sequence of SEQ ID NO:414; and (ii) a VL comprising a VL CDR1 having an amino acid sequence of SEQ ID NO:415, a VL CDR2 having an amino acid sequence of SEQ ID NO:416, and a VL CDR3 having an amino acid sequence of SEQ ID NO:417. In another aspect, provided herein is a TRGV9 antibody, wherein the antibody comprises: (i) a VH comprising a VH CDR1 having an amino acid sequence of SEQ ID NO:418, a VH CDR2 having an amino acid sequence of SEQ ID NO:419, and a VH CDR3 having an amino acid sequence of SEQ ID NO:420; and (ii) a VL comprising a VL CDR1 having an amino acid sequence of SEQ ID NO:421, a VL CDR2 having an amino acid sequence of SEQ ID NO:422, and a VL CDR3 having an amino acid sequence of SEQ ID NO:423. In another aspect, provided herein is a TRGV9 antibody, wherein the antibody comprises: (i) a VH comprising a VH CDR1 having an amino acid sequence of SEQ ID NO:424, a VH CDR2 having an amino acid sequence of SEQ ID NO:425, and a VH CDR3 having an amino acid sequence of SEQ ID NO:426; and (ii) a VL comprising a VL CDR1 having an amino acid sequence of SEQ ID NO:427, a VL CDR2 having an amino acid sequence of SEQ ID NO:428, and a VL CDR3 having an amino acid sequence of SEQ ID NO:429. In another aspect, provided herein is a TRGV9 antibody, wherein the antibody comprises: (i) a VH comprising a VH CDR1 having an amino acid sequence of SEQ ID NO:430, a VH CDR2 having an amino acid sequence of SEQ ID NO:431, and a VH CDR3 having an amino acid sequence of SEQ ID NO:432; and (ii) a VL comprising a VL CDR1 having an amino acid sequence of SEQ ID NO:433, a VL CDR2 having an amino acid sequence of SEQ ID NO:434, and a VL CDR3 having an amino acid sequence of SEQ ID NO:435. In another aspect, provided herein is a TRGV9 antibody, wherein the antibody comprises: (i) a VH comprising a VH CDR1 having an amino acid sequence of SEQ ID NO:430, a VH CDR2 having an amino acid sequence of SEQ ID NO:714, and a VH CDR3 having an amino acid sequence of SEQ ID NO:715; and (ii) a VL comprising a VL CDR1 having an amino acid sequence of SEQ ID NO:433, a VL CDR2 having an amino acid sequence of SEQ ID NO:434, and a VL CDR3 having an amino acid sequence of SEQ ID NO:435. In another aspect, provided herein is a TRGV9 antibody, wherein the antibody comprises: (i) a VH comprising a VH CDR1 having an amino acid sequence of SEQ ID NO:436, a VH CDR2 having an amino acid sequence of SEQ ID NO:437, and a VH CDR3 having an amino acid sequence of SEQ ID NO:438; and (ii) a VL comprising a VL CDR1 having an amino acid sequence of SEQ ID NO:439, a VL CDR2 having an amino acid sequence of SEQ ID NO:440, and a VL CDR3 having an amino acid sequence of SEQ ID NO:441. In another aspect, provided herein is a TRGV9 antibody, wherein the antibody comprises: (i) a VH comprising a VH CDR1 having an amino acid sequence of SEQ ID NO:442, a VH CDR2 having an amino acid sequence of SEQ ID NO:443, and a VH CDR3 having an amino acid sequence of SEQ ID NO:444; and (ii) a VL comprising a VL CDR1 having an amino acid sequence of SEQ ID NO:445, a VL CDR2 having an amino acid sequence of SEQ ID NO:446, and a VL CDR3 having an amino acid sequence of SEQ ID NO:447. In some embodiments, the antibody comprises a VH having an amino acid sequence of SEQ ID NO:104. In some embodiments, the antibody comprises a VL having an amino acid sequence of SEQ ID NO:105. In some embodiments, the antibody comprises a VH having an amino acid sequence of SEQ ID NO:104, and a VL having an amino acid sequence of SEQ ID NO:105. In some embodiments, the antibody comprises an amino acid sequence of SEQ ID NO:106. In some embodiments, the antibody comprises a VH comprising an amino acid sequence having at least 95% identity to the amino acid sequence of SEQ ID NO:104. In some embodiments, the antibody comprises a VL comprising an amino acid sequence having at least 95% identity to the amino acid sequence of SEQ ID NO:105. In some embodiments, the antibody comprises a VH comprising an amino acid sequence having at least 95% identity to the amino acid sequence of SEQ ID NO:104, and a VL comprising an amino acid sequence having at least 95% identity to the amino acid sequence of SEQ ID NO:105. In some embodiments, the antibody comprises an amino acid sequence of SEQ ID NO:106.

In one aspect, a TRGV9 antibody provided herein has a VH and VL amino acid sequence of VG9SB10SC1087_P18_D08. In one aspect, provided herein is a TRGV9 antibody, wherein the antibody comprises a VH comprising a VH CDR1, a VH CDR2, and a VH CDR3 having amino acid sequences of the VH CDR1, VH CDR2, and VH CDR3, respectively, of SEQ ID NO:113. In one aspect, provided herein is a TRGV9 antibody, wherein the antibody comprises a VL comprising a VL CDR1, a VL CDR2, and a VL CDR3 having amino acid sequences of the VL CDR1, VL CDR2, and VL CDR3, respectively, of SEQ ID NO:114. In another aspect, provided herein is a TRGV9 antibody, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:113; and (ii) a VL comprising a VL CDR1, a VL CDR2, and a VL CDR3 having amino acid sequences of the VL CDR1, VL CDR2, and VL CDR3, respectively, of SEQ ID NO:114. In another aspect, provided herein is a TRGV9 antibody, wherein the antibody comprises: (i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109; and (ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO:112. In another aspect, provided herein is a TRGV9 antibody, wherein the antibody comprises: (i) a VH comprising a VH CDR1 having an amino acid sequence of SEQ ID NO:448, a VH CDR2 having an amino acid sequence of SEQ ID NO:449, and a VH CDR3 having an amino acid sequence of SEQ ID NO:450; and (ii) a VL comprising a VL CDR1 having an amino acid sequence of SEQ ID NO:451, a VL CDR2 having an amino acid sequence of SEQ ID NO:452, and a VL CDR3 having an amino acid sequence of SEQ ID NO:453. In another aspect, provided herein is a TRGV9 antibody, wherein the antibody comprises: (i) a VH comprising a VH CDR1 having an amino acid sequence of SEQ ID NO:454, a VH CDR2 having an amino acid sequence of SEQ ID NO:455, and a VH CDR3 having an amino acid sequence of SEQ ID NO:456; and (ii) a VL comprising a VL CDR1 having an amino acid sequence of SEQ ID NO:457, a VL CDR2 having an amino acid sequence of SEQ ID NO:458, and a VL CDR3 having an amino acid sequence of SEQ ID NO:459. In another aspect, provided herein is a TRGV9 antibody, wherein the antibody comprises: (i) a VH comprising a VH CDR1 having an amino acid sequence of SEQ ID NO:460, a VH CDR2 having an amino acid sequence of SEQ ID NO:461, and a VH CDR3 having an amino acid sequence of SEQ ID NO:462; and (ii) a VL comprising a VL CDR1 having an amino acid sequence of SEQ ID NO:463, a VL CDR2 having an amino acid sequence of SEQ ID NO:464, and a VL CDR3 having an amino acid sequence of SEQ ID NO:465. In another aspect, provided herein is a TRGV9 antibody, wherein the antibody comprises: (i) a VH comprising a VH CDR1 having an amino acid sequence of SEQ ID NO:466, a VH CDR2 having an amino acid sequence of SEQ ID NO:467, and a VH CDR3 having an amino acid sequence of SEQ ID NO:468; and (ii) a VL comprising a VL CDR1 having an amino acid sequence of SEQ ID NO:469, a VL CDR2 having an amino acid sequence of SEQ ID NO:470, and a VL CDR3 having an amino acid sequence of SEQ ID NO:471. In another aspect, provided herein is a TRGV9 antibody, wherein the antibody comprises: (i) a VH comprising a VH CDR1 having an amino acid sequence of SEQ ID NO:466, a VH CDR2 having an amino acid sequence of SEQ ID NO:716, and a VH CDR3 having an amino acid sequence of SEQ ID NO:717; and (ii) a VL comprising a VL CDR1 having an amino acid sequence of SEQ ID NO:469, a VL CDR2 having an amino acid sequence of SEQ ID NO:470, and a VL CDR3 having an amino acid sequence of SEQ ID NO:471. In another aspect, provided herein is a TRGV9 antibody, wherein the antibody comprises: (i) a VH comprising a VH CDR1 having an amino acid sequence of SEQ ID NO:472, a VH CDR2 having an amino acid sequence of SEQ ID NO:473, and a VH CDR3 having an amino acid sequence of SEQ ID NO:474; and (ii) a VL comprising a VL CDR1 having an amino acid sequence of SEQ ID NO:475, a VL CDR2 having an amino acid sequence of SEQ ID NO:476, and a VL CDR3 having an amino acid sequence of SEQ ID NO:477. In another aspect, provided herein is a TRGV9 antibody, wherein the antibody comprises: (i) a VH comprising a VH CDR1 having an amino acid sequence of SEQ ID NO:478, a VH CDR2 having an amino acid sequence of SEQ ID NO:479, and a VH CDR3 having an amino acid sequence of SEQ ID NO:480; and (ii) a VL comprising a VL CDR1 having an amino acid sequence of SEQ ID NO:481, a VL CDR2 having an amino acid sequence of SEQ ID NO:482, and a VL CDR3 having an amino acid sequence of SEQ ID NO:483. In some embodiments, the antibody comprises a VH having an amino acid sequence of SEQ ID NO:113. In some embodiments, the antibody comprises a VL having an amino acid sequence of SEQ ID NO:114. In some embodiments, the antibody comprises a VH having an amino acid sequence of SEQ ID NO:113, and a VL having an amino acid sequence of SEQ ID NO:114. In some embodiments, the antibody comprises a heavy chain having an amino acid sequence of SEQ ID NO:115. In some embodiments, the antibody comprises a light chain having an amino acid sequence of SEQ ID NO:116. In some embodiments, the antibody comprises a heavy chain having an amino acid sequence of SEQ ID NO:115, and a light chain having an amino acid sequence of SEQ ID NO:116. In some embodiments, the antibody comprises a VH comprising an amino acid sequence having at least 95% identity to the amino acid sequence of SEQ ID NO:113. In some embodiments, the antibody comprises a VL comprising an amino acid sequence having at least 95% identity to the amino acid sequence of SEQ ID NO:114. In some embodiments, the antibody comprises a VH comprising an amino acid sequence having at least 95% identity to the amino acid sequence of SEQ ID NO:113, and a VL comprising an amino acid sequence having at least 95% identity to the amino acid sequence of SEQ ID NO:114. In some embodiments, the antibody comprises a heavy chain comprising an amino acid sequence having at least 95% identity to the amino acid sequence of SEQ ID NO:115. In some embodiments, the antibody comprises a light chain comprising an amino acid sequence having at least 95% identity to the amino acid sequence of SEQ ID NO:116. In some embodiments, the antibody comprises a heavy chain comprising an amino acid sequence having at least 95% identity to the amino acid sequence of SEQ ID NO:115, and a light chain comprising an amino acid sequence having at least 95% identity to the amino acid sequence of SEQ ID NO:116.

In one aspect, a TRGV9 antibody provided herein has a VH and VL amino acid sequence of VG9SB10SC1087_P18_C12. In one aspect, provided herein is a TRGV9 antibody, wherein the antibody comprises a VH comprising a VH CDR1, a VH CDR2, and a VH CDR3 having amino acid sequences of the VH CDR1, VH CDR2, and VH CDR3, respectively, of SEQ ID NO:123. In one aspect, provided herein is a TRGV9 antibody, wherein the antibody comprises a VL comprising a VL CDR1, a VL CDR2, and a VL CDR3 having amino acid sequences of the VL CDR1, VL CDR2, and VL CDR3, respectively, of SEQ ID NO:124. In another aspect, provided herein is a TRGV9 antibody, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:123; and (ii) a VL comprising a VL CDR1, a VL CDR2, and a VL CDR3 having amino acid sequences of the VL CDR1, VL CDR2, and VL CDR3, respectively, of SEQ ID NO:124. In another aspect, provided herein is a TRGV9 antibody, wherein the antibody comprises: (i) a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119; and (ii) a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO:122. In another aspect, provided herein is a TRGV9 antibody, wherein the antibody comprises: (i) a VH comprising a VH CDR1 having an amino acid sequence of SEQ ID NO:484, a VH CDR2 having an amino acid sequence of SEQ ID NO:485, and a VH CDR3 having an amino acid sequence of SEQ ID NO:486; and (ii) a VL comprising a VL CDR1 having an amino acid sequence of SEQ ID NO:487, a VL CDR2 having an amino acid sequence of SEQ ID NO:488, and a VL CDR3 having an amino acid sequence of SEQ ID NO:489. In another aspect, provided herein is a TRGV9 antibody, wherein the antibody comprises: (i) a VH comprising a VH CDR1 having an amino acid sequence of SEQ ID NO:490, a VH CDR2 having an amino acid sequence of SEQ ID NO:491, and a VH CDR3 having an amino acid sequence of SEQ ID NO:492; and (ii) a VL comprising a VL CDR1 having an amino acid sequence of SEQ ID NO:493, a VL CDR2 having an amino acid sequence of SEQ ID NO:494, and a VL CDR3 having an amino acid sequence of SEQ ID NO:495. In another aspect, provided herein is a TRGV9 antibody, wherein the antibody comprises: (i) a VH comprising a VH CDR1 having an amino acid sequence of SEQ ID NO:496, a VH CDR2 having an amino acid sequence of SEQ ID NO:497, and a VH CDR3 having an amino acid sequence of SEQ ID NO:498; and (ii) a VL comprising a VL CDR1 having an amino acid sequence of SEQ ID NO:499, a VL CDR2 having an amino acid sequence of SEQ ID NO:500, and a VL CDR3 having an amino acid sequence of SEQ ID NO:501. In another aspect, provided herein is a TRGV9 antibody, wherein the antibody comprises: (i) a VH comprising a VH CDR1 having an amino acid sequence of SEQ ID NO:502, a VH CDR2 having an amino acid sequence of SEQ ID NO:503, and a VH CDR3 having an amino acid sequence of SEQ ID NO:504; and (ii) a VL comprising a VL CDR1 having an amino acid sequence of SEQ ID NO:505, a VL CDR2 having an amino acid sequence of SEQ ID NO:506, and a VL CDR3 having an amino acid sequence of SEQ ID NO:507. In another aspect, provided herein is a TRGV9 antibody, wherein the antibody comprises: (i) a VH comprising a VH CDR1 having an amino acid sequence of SEQ ID NO:502, a VH CDR2 having an amino acid sequence of SEQ ID NO:718, and a VH CDR3 having an amino acid sequence of SEQ ID NO:719; and (ii) a VL comprising a VL CDR1 having an amino acid sequence of SEQ ID NO:505, a VL CDR2 having an amino acid sequence of SEQ ID NO:506, and a VL CDR3 having an amino acid sequence of SEQ ID NO:507. In another aspect, provided herein is a TRGV9 antibody, wherein the antibody comprises: (i) a VH comprising a VH CDR1 having an amino acid sequence of SEQ ID NO:508, a VH CDR2 having an amino acid sequence of SEQ ID NO:509, and a VH CDR3 having an amino acid sequence of SEQ ID NO:510; and (ii) a VL comprising a VL CDR1 having an amino acid sequence of SEQ ID NO:511, a VL CDR2 having an amino acid sequence of SEQ ID NO:512, and a VL CDR3 having an amino acid sequence of SEQ ID NO:513. In another aspect, provided herein is a TRGV9 antibody, wherein the antibody comprises: (i) a VH comprising a VH CDR1 having an amino acid sequence of SEQ ID NO:514, a VH CDR2 having an amino acid sequence of SEQ ID NO:515, and a VH CDR3 having an amino acid sequence of SEQ ID NO:516; and (ii) a VL comprising a VL CDR1 having an amino acid sequence of SEQ ID NO:517, a VL CDR2 having an amino acid sequence of SEQ ID NO:518, and a VL CDR3 having an amino acid sequence of SEQ ID NO:519. In some embodiments, the antibody comprises a VH having an amino acid sequence of SEQ ID NO:123. In some embodiments, the antibody comprises a VL having an amino acid sequence of SEQ ID NO:124. In some embodiments, the antibody comprises a VH having an amino acid sequence of SEQ ID NO:123, and a VL having an amino acid sequence of SEQ ID NO:124. In some embodiments, the antibody comprises a heavy chain having an amino acid sequence of SEQ ID NO:125. In some embodiments, the antibody comprises a light chain having an amino acid sequence of SEQ ID NO:126. In some embodiments, the antibody comprises a heavy chain having an amino acid sequence of SEQ ID NO:125, and a light chain having an amino acid sequence of SEQ ID NO:126. In some embodiments, the antibody comprises a VH comprising an amino acid sequence having at least 95% identity to the amino acid sequence of SEQ ID NO:123. In some embodiments, the antibody comprises a VL comprising an amino acid sequence having at least 95% identity to the amino acid sequence of SEQ ID NO:124. In some embodiments, the antibody comprises a VH comprising an amino acid sequence having at least 95% identity to the amino acid sequence of SEQ ID NO:123, and a VL comprising an amino acid sequence having at least 95% identity to the amino acid sequence of SEQ ID NO:124. In some embodiments, the antibody comprises a heavy chain comprising an amino acid sequence having at least 95% identity to the amino acid sequence of SEQ ID NO:125. In some embodiments, the antibody comprises a light chain comprising an amino acid sequence having at least 95% identity to the amino acid sequence of SEQ ID NO:126. In some embodiments, the antibody comprises a heavy chain comprising an amino acid sequence having at least 95% identity to the amino acid sequence of SEQ ID NO:125, and a light chain comprising an amino acid sequence having at least 95% identity to the amino acid sequence of SEQ ID NO:126.

In one aspect, a TRGV9 antibody provided herein has a VH and VL amino acid sequence of VG9SB10SC1087_P19_C03. In one aspect, provided herein is a TRGV9 antibody, wherein the antibody comprises a VH comprising a VH CDR1, a VH CDR2, and a VH CDR3 having amino acid sequences of the VH CDR1, VH CDR2, and VH CDR3, respectively, of SEQ ID NO:133. In one aspect, provided herein is a TRGV9 antibody, wherein the antibody comprises a VL comprising a VL CDR1, a VL CDR2, and a VL CDR3 having amino acid sequences of the VL CDR1, VL CDR2, and VL CDR3, respectively, of SEQ ID NO:134. In another aspect, provided herein is a TRGV9 antibody, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:133; and (ii) a VL comprising a VL CDR1, a VL CDR2, and a VL CDR3 having amino acid sequences of the VL CDR1, VL CDR2, and VL CDR3, respectively, of SEQ ID NO:134. In another aspect, provided herein is a TRGV9 antibody, wherein the antibody comprises: (i) a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO:129; and (ii) a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO:132. In another aspect, provided herein is a TRGV9 antibody, wherein the antibody comprises: (i) a VH comprising a VH CDR1 having an amino acid sequence of SEQ ID NO:520, a VH CDR2 having an amino acid sequence of SEQ ID NO:521, and a VH CDR3 having an amino acid sequence of SEQ ID NO:522; and (ii) a VL comprising a VL CDR1 having an amino acid sequence of SEQ ID NO:523, a VL CDR2 having an amino acid sequence of SEQ ID NO:524, and a VL CDR3 having an amino acid sequence of SEQ ID NO:525. In another aspect, provided herein is a TRGV9 antibody, wherein the antibody comprises: (i) a VH comprising a VH CDR1 having an amino acid sequence of SEQ ID NO:526, a VH CDR2 having an amino acid sequence of SEQ ID NO:527, and a VH CDR3 having an amino acid sequence of SEQ ID NO:528; and (ii) a VL comprising a VL CDR1 having an amino acid sequence of SEQ ID NO:529, a VL CDR2 having an amino acid sequence of SEQ ID NO:530, and a VL CDR3 having an amino acid sequence of SEQ ID NO:531. In another aspect, provided herein is a TRGV9 antibody, wherein the antibody comprises: (i) a VH comprising a VH CDR1 having an amino acid sequence of SEQ ID NO:532, a VH CDR2 having an amino acid sequence of SEQ ID NO:533, and a VH CDR3 having an amino acid sequence of SEQ ID NO:534; and (ii) a VL comprising a VL CDR1 having an amino acid sequence of SEQ ID NO:535, a VL CDR2 having an amino acid sequence of SEQ ID NO:536, and a VL CDR3 having an amino acid sequence of SEQ ID NO:537. In another aspect, provided herein is a TRGV9 antibody, wherein the antibody comprises: (i) a VH comprising a VH CDR1 having an amino acid sequence of SEQ ID NO:538, a VH CDR2 having an amino acid sequence of SEQ ID NO:539, and a VH CDR3 having an amino acid sequence of SEQ ID NO:540; and (ii) a VL comprising a VL CDR1 having an amino acid sequence of SEQ ID NO:541, a VL CDR2 having an amino acid sequence of SEQ ID NO:542, and a VL CDR3 having an amino acid sequence of SEQ ID NO:543. In another aspect, provided herein is a TRGV9 antibody, wherein the antibody comprises: (i) a VH comprising a VH CDR1 having an amino acid sequence of SEQ ID NO:538, a VH CDR2 having an amino acid sequence of SEQ ID NO:720, and a VH CDR3 having an amino acid sequence of SEQ ID NO:721; and (ii) a VL comprising a VL CDR1 having an amino acid sequence of SEQ ID NO:541, a VL CDR2 having an amino acid sequence of SEQ ID NO:542, and a VL CDR3 having an amino acid sequence of SEQ ID NO:543. In another aspect, provided herein is a TRGV9 antibody, wherein the antibody comprises: (i) a VH comprising a VH CDR1 having an amino acid sequence of SEQ ID NO:544, a VH CDR2 having an amino acid sequence of SEQ ID NO:545, and a VH CDR3 having an amino acid sequence of SEQ ID NO:546; and (ii) a VL comprising a VL CDR1 having an amino acid sequence of SEQ ID NO:547, a VL CDR2 having an amino acid sequence of SEQ ID NO:548, and a VL CDR3 having an amino acid sequence of SEQ ID NO:549. In another aspect, provided herein is a TRGV9 antibody, wherein the antibody comprises: (i) a VH comprising a VH CDR1 having an amino acid sequence of SEQ ID NO:550, a VH CDR2 having an amino acid sequence of SEQ ID NO:551, and a VH CDR3 having an amino acid sequence of SEQ ID NO:552; and (ii) a VL comprising a VL CDR1 having an amino acid sequence of SEQ ID NO:553, a VL CDR2 having an amino acid sequence of SEQ ID NO:554, and a VL CDR3 having an amino acid sequence of SEQ ID NO:555. In some embodiments, the antibody comprises a VH having an amino acid sequence of SEQ ID NO:133. In some embodiments, the antibody comprises a VL having an amino acid sequence of SEQ ID NO:134. In some embodiments, the antibody comprises a VH having an amino acid sequence of SEQ ID NO:133, and a VL having an amino acid sequence of SEQ ID NO:134. In some embodiments, the antibody comprises a heavy chain having an amino acid sequence of SEQ ID NO:135. In some embodiments, the antibody comprises a light chain having an amino acid sequence of SEQ ID NO:136. In some embodiments, the antibody comprises a heavy chain having an amino acid sequence of SEQ ID NO:135, and a light chain having an amino acid sequence of SEQ ID NO:136. In some embodiments, the antibody comprises a VH comprising an amino acid sequence having at least 95% identity to the amino acid sequence of SEQ ID NO:133. In some embodiments, the antibody comprises a VL comprising an amino acid sequence having at least 95% identity to the amino acid sequence of SEQ ID NO:134. In some embodiments, the antibody comprises a VH comprising an amino acid sequence having at least 95% identity to the amino acid sequence of SEQ ID NO:133, and a VL comprising an amino acid sequence having at least 95% identity to the amino acid sequence of SEQ ID NO:134. In some embodiments, the antibody comprises a heavy chain comprising an amino acid sequence having at least 95% identity to the amino acid sequence of SEQ ID NO:135. In some embodiments, the antibody comprises a light chain comprising an amino acid sequence having at least 95% identity to the amino acid sequence of SEQ ID NO:136. In some embodiments, the antibody comprises a heavy chain comprising an amino acid sequence having at least 95% identity to the amino acid sequence of SEQ ID NO:135, and a light chain comprising an amino acid sequence having at least 95% identity to the amino acid sequence of SEQ ID NO:136.

In some embodiments, the VH CDR1, VH CDR2 and VH CDR3 amino acid sequences of the TRGV9 antibody are according to the Kabat numbering system. In some embodiments, the VH CDR1, VH CDR2 and VH CDR3 amino acid sequences of the TRGV9 antibody are according to the Chothia numbering system. In some embodiments, the VH CDR1, VH CDR2 and VH CDR3 amino acid sequences of the TRGV9 antibody are according to the AbM numbering system. In some embodiments, the VH CDR1, VH CDR2 and VH CDR3 amino acid sequences of the TRGV9 antibody are according to the Contact numbering system. In some embodiments, the VH CDR1, VH CDR2 and VH CDR3 amino acid sequences of the TRGV9 antibody are according to the IMGT numbering system. In some embodiments, the VH CDR1, VH CDR2 and VH CDR3 amino acid sequences of the TRGV9 antibody are according to the Exemplary numbering system. In some embodiments, the VL CDR1, VL CDR2 and VL CDR3 amino acid sequences of the TRGV9 antibody are according to the Kabat numbering system. In some embodiments, the VL CDR1, VL CDR2 and VL CDR3 amino acid sequences of the TRGV9 antibody are according to the Chothia numbering system. In some embodiments, the VL CDR1, VL CDR2 and VL CDR3 amino acid sequences of the TRGV9 antibody are according to the AbM numbering system. In some embodiments, the VL CDR1, VL CDR2 and VL CDR3 amino acid sequences of the TRGV9 antibody are according to the Contact numbering system. In some embodiments, the VL CDR1, VL CDR2 and VL CDR3 amino acid sequences of the TRGV9 antibody are according to the IMGT numbering system. In some embodiments, the VL CDR1, VL CDR2 and VL CDR3 amino acid sequences of the TRGV9 antibody are according to the Exemplary numbering system.

In some embodiments, the TRGV9 antibody is a multispecific antibody. In other embodiments, the TRGV9 antibody is a bispecific antibody. In certain embodiments, the multispecific antibody comprises an antigen binding fragment of a TRGV9 antibody provided herein. In some embodiments, the multispecific antibody comprises a first binding domain that binds to a first TRGV9 epitope and a second domain that binds to a second TRGV9 epitope, wherein the first TRGV9 epitope and the second TRGV9 epitope are different. In certain embodiments, the multispecific antibody further comprises a third binding domain that binds to a target that is not TRGV9. In some embodiments, the multispecific antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In some embodiments, the first binding domain of the TRGV9 antibody is not a single domain antibody or nanobody. In a some embodiments, the second binding domain of the TRGV9 antibody is not a single domain antibody or nanobody.

In specific embodiments, the TRGV9 antibody comprises a VH region and a VL region. In some embodiments, the TRGV9 antibody is not a single chain antibody. In some embodiments, the TRGV9 antibody is not a single domain antibody. In some embodiments, the TRGV9 antibody is not a nanobody. In certain embodiments, the TRGV9 antibody is not a VHH antibody. In certain embodiments, the TRGV9 antibody is not a llama antibody. In some embodiments, the TRGV9 bispecific antibody does not comprise a single chain antibody. In some embodiments, the TRGV9 bispecific antibody does not comprise a single domain antibody. In certain embodiments, the TRGV9 bispecific antibody does not comprise a nanobody. In certain embodiments, the TRGV9 bispecific antibody does not comprise a VHH antibody. In certain embodiments, the TRGV9 bispecific antibody does not comprise a llama antibody.

In some embodiments, a TRGV9 antibody provided herein does not comprise a VH CDR1, VH CDR2, and VH CDR3 having the amino acid sequence of SEQ ID NOs:730, 731, and 732, respectively. In some embodiments, a TRGV9 antibody provided herein does not comprise a VH CDR1, VH CDR2, and VH CDR3 having the amino acid sequence of SEQ ID NOs:733, 734, and 735, respectively. In some embodiments, a TRGV9 antibody provided herein does not comprise a VH CDR1, VH CDR2, and VH CDR3 having the amino acid sequence of SEQ ID NOs:736, 737, and 738, respectively. In some embodiments, a TRGV9 antibody provided herein does not comprise a VH CDR1, VH CDR2, and VH CDR3 having the amino acid sequence of SEQ ID NOs:739, 740, and 741, respectively. In some embodiments, a TRGV9 antibody provided herein does not comprise a VH CDR1, VH CDR2, and VH CDR3 having the amino acid sequence of SEQ ID NOs:742, 743, and 744, respectively. In some embodiments, a TRGV9 antibody provided herein does not comprise a VH CDR1, VH CDR2, and VH CDR3 having the amino acid sequence of SEQ ID NOs:745, 746, and 747, respectively. In some embodiments, a TRGV9 antibody provided herein does not comprise a VH CDR1, VH CDR2, and VH CDR3 having the amino acid sequence of SEQ ID NOs:748, 749, and 750, respectively. In some embodiments, a TRGV9 antibody provided herein does not comprise a VH domain having the amino acid sequence of SEQ ID NO:751. In some embodiments, a TRGV9 antibody provided herein does not comprise a VH domain having the amino acid sequence of SEQ ID NO:752. In some embodiments, a TRGV9 antibody provided herein does not comprise a VH domain having the amino acid sequence of SEQ ID NO:753. In some embodiments, a TRGV9 antibody provided herein does not comprise a VH domain having the amino acid sequence of SEQ ID NO:754. In some embodiments, a TRGV9 antibody provided herein does not comprise a VH domain having the amino acid sequence of SEQ ID NO:755. In some embodiments, a TRGV9 antibody provided herein does not comprise a VH domain having the amino acid sequence of SEQ ID NO:756. In some embodiments, a TRGV9 antibody provided herein does not comprise a VH domain having the amino acid sequence of SEQ ID NO:757.

In another aspect, provided herein is a TRGV9 antibody, comprising a VH domain comprising a VH CDR3 having the amino acid sequence of APNxGzYTbDF (SEQ ID NO:758), wherein x is Y or M, z is M or D, and b is I or L. In another aspect, provided herein is a TRGV9 antibody, comprising a VH domain comprising the amino acid sequence of SEQ ID NO:758. In another aspect, provided herein is a TRGV9 antibody, comprising a VH domain comprising a VH CDR1 having the amino acid sequence of GxTFzz (SEQ ID NO:761), wherein xis F, D or G, and z is S or N. In another aspect, provided herein is a TRGV9 antibody, comprising a VH domain comprising the amino acid sequence of SEQ ID NO:761. In another aspect, provided herein is a TRGV9 antibody, comprising a VL domain comprising a VL CDR1 having the amino acid sequence of RxSQSz (SEQ ID NO:762), wherein x is A or S, and z is V or L. In another aspect, provided herein is a TRGV9 antibody, comprising a VL domain comprising the amino acid sequence of SEQ ID NO:761.

In another aspect, provided herein is an antibody that binds to TRGV9, wherein the antibody comprises: (i) a VH comprising a VH CDR1, a VH CDR2, and a VH CDR3; and (ii) a VL comprising a VL CDR1, a VL CDR2, and a VL CDR3. In some embodiments, the VH CDR1 comprises a first polar amino acid. In some embodiments, the VH CDR1 comprises a last polar uncharged amino acid. In some embodiments, the VH CDR1 comprises at least one tyrosine. In some embodiments, the VH CDR1 comprises at least 20% hydrophobic amino acids. In some embodiments, the VH CDR1 comprises at least two hydrophobic amino acids. In some embodiments, the VH CDR1 comprises at least about 40% hydrophobic amino acids. In some embodiments, the VH CDR1 comprises the VH CDR1 comprises a first polar amino acid, a last polar uncharged amino acid, at least one tyrosine, at least 20% hydrophobic amino acids, at least two hydrophobic amino acids, and at least about 40% hydrophobic amino acids. Any combination of two or more of the above-mentioned VH CDR1 structural features are also contemplated. In some embodiments, the VH CDR2 comprises a polar amino acid at residue 13. In some embodiments, the VH CDR2 comprises a hydrophobic at amino acid position 15. In some embodiments, the VH CDR2 comprises a phenylalanine (F) or leucine (L) at position 15. In some embodiments, the VH CDR2 comprises a polar amino acid at position 14. In some embodiments, the VH CDR2 comprises a lysine (K) or serine (S) at position 14. In some embodiments, the VH CDR2 comprises a hydrophobic amino acid at position 2. In some embodiments, the VH CDR2 comprises a hydrophobic amino acid at position 3. In some embodiments, the VH CDR2 comprises and a polar penultimate amino acid. In some embodiments, the VH CDR2 comprises a polar amino acid at residue 13, a hydrophobic at amino acid position 15, a phenylalanine (F) or leucine (L) at position 15, a polar amino acid at position 14, a lysine (K) or serine (S) at position 14, a hydrophobic amino acid at position 2 or 3, and a polar penultimate amino acid. Any combination of two or more of the above-mentioned VH CDR2 structural features are also contemplated. In some embodiments, the VH CDR3 does not comprise a polar charged amino acid at position 3. In some embodiments, the VH CDR3 comprises a hydrophobic or polar charged amino acid at position 7. In some embodiments, the VH CDR3 comprises a polar uncharged or hydrophobic amino acid at position 6. In some embodiments, the VH CDR3 comprises no polar charged amino acid at position 3, a hydrophobic or polar charged amino acid at position 7, and a polar uncharged or hydrophobic amino acid at position 6. Any combination of two or more of the above-mentioned VH CDR3 structural features are also contemplated. In some embodiments, the VL CDR1 comprises a polar amino acid at position 4. In some embodiments, the VL CDR1 comprises a first amino acid that is polar charged. In some embodiments, the VL CDR1 comprises a polar uncharged or hydrophobic amino acid at position 2. In some embodiments, the VL CDR1 comprises a serine at position 3. In some embodiments, the VL CDR1 comprises a polar amino acid at position 5. In some embodiments, the VL CDR1 comprises a hydrophobic amino acid at position 6. In some embodiments, the VL CDR1 comprises a polar amino acid at position 4, a first amino acid that is polar charged, a polar uncharged or hydrophobic amino acid at position 2, a serine at position 3, a polar amino acid at position 5, and a hydrophobic amino acid at position 6. Any combination of two or more of the above-mentioned VL CDR1 structural features are also contemplated. In some embodiments, the VL CDR2 comprises a polar amino acid at position 7. In some embodiments, the VL CDR2 comprises a polar charged or hydrophobic amino acid at position 6. In some embodiments, the VL CDR2 comprises a polar charged amino acid at position 3. In some embodiments, the VL CDR2 comprises a polar uncharged amino acid at position 4. In some embodiments, the VL CDR2 comprises a hydrophobic amino acid at position 2. In some embodiments, the VL CDR2 comprises a polar amino acid at position 7, a polar charged or hydrophobic amino acid at position 6, a polar charged amino acid at position 3, a polar uncharged amino acid at position 4, and a hydrophobic amino acid at position 2. Any combination of two or more of the above-mentioned VL CDR2 structural features are also contemplated. In some embodiments, the VL CDR3 comprises a hydrophobic terminal amino acid. In some embodiments, the VL CDR3 comprises a terminal tyrosine. In some embodiments, the VL CDR3 comprises a polar uncharged amino acid at position 5. In some embodiments, the VL CDR3 comprises a polar amino acid at position 2. In some embodiments, the VL CDR3 comprises a polar uncharged or hydrophobic amino acid at position 1. In some embodiments, the VL CDR3 comprises a hydrophobic amino acid at position 3. In some embodiments, the VL CDR3 comprises a hydrophilic or polar uncharged amino acid at position 6. In some embodiments, the VL CDR3 comprises no polar or hydrophobic amino acid at position 7. In some embodiments, the VL CDR3 comprises a hydrophobic terminal amino acid, a terminal tyrosine, a polar uncharged amino acid at position 5, a polar amino acid at position 2, a polar uncharged or hydrophobic amino acid at position 1, a hydrophobic amino acid at position 3, a hydrophilic or polar uncharged amino acid at position 6, and no polar or hydrophobic amino acid at position 7. Any combination of two or more of the above-mentioned VL CDR3 structural features are also contemplated. In specific embodiments, residue position numbering is according to Exemplary numbering.

In another aspect, provided herein is an antibody that binds to TRGV9, wherein the antibody comprises: (i) a VH comprising a VH CDR1, a VH CDR2, and a VH CDR3; and (ii) a VL comprising a VL CDR1, a VL CDR2, and a VL CDR3.

In some embodiments of the TRGV9 antibodies provided herein, the VH CDR1 comprises at least three polar amino acids. In some embodiments, the VH CDR1 comprises at least 40% polar amino acids. In some embodiments, the VH CDR1 comprises a glycine (G) at position 1. In some embodiments, the VH CDR1 does not comprise a polar uncharged amino acid at position 2. In some embodiments, the VH CDR1 comprises a polar uncharged amino acid at position 3. In some embodiments, the VH CDR1 comprises a threonine (T) or a serine (S) at position 3. In some embodiments, the VH CDR1 comprises a hydrophobic amino acid at position 4. In some embodiments, the VH CDR1 comprises a phenylalanine (F) or an isoleucine (I) at position 4. In some embodiments, the VH CDR1 comprises a polar uncharged amino acid at position 5. In some embodiments, the VH CDR1 comprises a threonine (T), a serine (S), or an asparagine (N) at position 5. In some embodiments, the VH CDR1 comprises a polar amino acid at position 6. In some embodiments, the VH CDR1 comprises an acid amino acid or a polar uncharged amino acid at position 6. In some embodiments, the VH CDR1 does not comprise a polar uncharged amino acid at position 7. Any combination of two or more of the above-mentioned VH CDR1 structural features are also contemplated. In certain embodiments, the amino acid residue numbering is according to Chothia.

In some embodiments of the TRGV9 antibodies provided herein, the VH CDR2 does not comprise a charged amino acid at position 1. In some embodiments, the VH CDR2 does not comprise a charged amino acid at position 2. In some embodiments, the VH CDR2 comprises a glycine (G), a hydrophobic, or a polar uncharged amino acid at position 2. In some embodiments, the VH CDR2 does not comprises a hydrophobic or a polar charged amino acid at position 4. In some embodiments, the VH CDR2 does not comprise a hydrophobic or a polar uncharged amino acid at the last position. In some embodiments, the VH CDR2 comprises a glycine (G) or a polar uncharged amino acid at the last position. Any combination of two or more of the above-mentioned VH CDR2 structural features are also contemplated. In certain embodiments, the amino acid residue numbering is according to Chothia.

In some embodiments of the TRGV9 antibodies provided herein, the VH CDR3 does not comprise a polar charged amino acid at position 1. In some embodiments, the VH CDR3 does not comprise a polar charged amino acid at position 2. In some embodiments, the VH CDR3 comprises a glycine (G), a tyrosine (Y), or a polar uncharged amino acid at position 2. In some embodiments, the VH CDR3 does not comprise a polar uncharged amino acid at position 3. In some embodiments, the VH CDR3 comprises a glycine (G), an aspartic acid (D), or a hydrophobic amino acid at position 3. In some embodiments, the VH CDR3 does not comprise a polar charged amino acid at position 5. In some embodiments, the VH CDR3 does not comprise a polar uncharged amino acid at position 6. In some embodiments, the VH CDR3 comprises an aspartic acid (D) or a hydrophobic amino acid at position 6. In some embodiments, the VH CDR3 comprises a hydrophobic penultimate amino acid. In some embodiments, the VH CDR3 does not comprise a polar uncharged amino acid at position 7. In some embodiments, the VH CDR3 comprises a terminal aspartic acid (D) or a terminal alanine (A). Any combination of two or more of the above-mentioned VH CDR3 structural features are also contemplated. In certain embodiments, the amino acid residue numbering is according to Chothia.

In some embodiments of the TRGV9 antibodies provided herein, the VL CDR1 comprises a serine (S) at position 1. In some embodiments, the VL CDR1 comprises a glutamine (G) or a glutamic acid (E) at position 2. In some embodiments, the VL CDR1 comprises a polar uncharged amino acid at position 3. In some embodiments, the VL CDR1 comprises a serine (S) or an asparagine (N) at position 3. In some embodiments, the VL CDR1 comprises a hydrophobic amino acid at position 4. In some embodiments, the VL CDR1 comprises a leucine (L), valine (V), or isoleucine (I) at position 4. In some embodiments, the VL CDR1 comprises a serine (S) or tyrosine (Y) at position 7. In some embodiments, the VL CDR1 comprises a polar uncharged penultimate amino acid. In some embodiments, the VL CDR1 comprises a terminal tyrosine (Y) or a terminal lysine (K). Any combination of two or more of the above-mentioned VL CDR1 structural features are also contemplated. In certain embodiments, the amino acid residue numbering is according to Chothia.

In some embodiments of the TRGV9 antibodies provided herein, the VL CDR2 comprises a hydrophobic amino acid at position 2. In some embodiments, the VL CDR2 comprises an alanine (A) or an isoleucine (I) at position 2. In some embodiments, the VL CDR2 comprises a polar terminal amino acid. In some embodiments, the VL CDR2 comprises a terminal serine (S) or a terminal lysine (K). Any combination of two or more of the above-mentioned VL CDR2 structural features are also contemplated. In certain embodiments, the amino acid residue numbering is according to Chothia.

In some embodiments of the TRGV9 antibodies provided herein, the VL CDR3 does not comprise a polar uncharged amino acid at position 1. In some embodiments, the VL CDR3 comprises an arginine (R) or a hydrophobic amino acid at position 1. In some embodiments, the VL CDR3 does not comprise a hydrophobic amino acid at position 3. In some embodiments, the VL CDR3 comprises an arginine (R) or a polar uncharged amino acid at position 3. In some embodiments, the VL CDR3 does not comprise a polar charged amino acid at position 4. In some embodiments, the VL CDR3 comprises a serine (S) or a hydrophobic amino acid at position 4. In some embodiments, the VL CDR3 comprises a tyrosine (Y) or a proline (P) as the penultimate amino acid. In some embodiments, the VL CDR3 comprises a histidine (H) or a proline (P) at position 5. In some embodiments, the VL CDR3 comprises a terminal histidine (H), a terminal lysine (L), or a terminal tyrosine (Y). Any combination of two or more of the above-mentioned VL CDR3 structural features are also contemplated. In certain embodiments, the amino acid residue numbering is according to Chothia.

In another aspect, provided herein is a TRGV9 antibody, comprising a VH comprising a VH CDR1 having the amino acid sequence of GX₁TFX₂×₃×₄ (SEQ ID NO:777), wherein X₁ is F, D, or G; X₂ is T, S, or N; X₃ is D, S, or N; and X₄ is H, N, or Y. In another aspect, provided herein is a TRGV9 antibody, comprising a VH comprising the amino acid sequence of SEQ ID NO:777. In another aspect, provided herein is a TRGV9 antibody, comprising a VH comprising a VH CDR2 having the amino acid sequence of PGX₁G (SEQ ID NO:778), wherein X₁ is D or S. In another aspect, provided herein is a TRGV9 antibody, comprising a VH comprising the amino acid sequence of SEQ ID NO:778. In another aspect, provided herein is a TRGV9 antibody, comprising a VH comprising a VH CDR3 having the amino acid sequence of X₁GX₂YTX₃D (SEQ ID NO:779), wherein X₁ is Y or M, X₂ is D or M, and X₃ is I or L. In another aspect, provided herein is a TRGV9 antibody, comprising a VH comprising the amino acid sequence of SEQ ID NO:779. In another aspect, provided herein is a TRGV9 antibody, comprising a VL comprising a VL CDR1 having the amino acid sequence of SQSX₁LYSSNX₂×₃ (SEQ ID NO:780), wherein X₁ is L or V, X₂ is Q or N, and X₃ is K or KNY. In another aspect, provided herein is a TRGV9 antibody, comprising a VL comprising the amino acid sequence of SEQ ID NO:780.

In another aspect, provided herein is a TRGV9 antibody, comprising a VH comprising a VH CDR1, VH CDR2 and VH CDR3. In some embodiments, the VH CDR1 comprises an amino acid sequence of GX₁TFX₂×₃×₄ (SEQ ID NO:777), wherein X₁ is F, D, or G; X₂ is T, S, or N; X₃ is D, S, or N; and X₄ is H, N, or Y. In some embodiments, the VH CDR1 comprises an amino acid sequences o SEQ ID NO:178. In some embodiments, the VH CDR1 comprises an amino acid sequences o SEQ ID NO:394. In some embodiments, the VH CDR1 comprises an amino acid sequences o SEQ ID NO:430. In some embodiments, the VH CDR1 comprises an amino acid sequences o SEQ ID NO:466. In some embodiments, the VH CDR1 comprises an amino acid sequences o SEQ ID NO:502. In some embodiments, the VH CDR1 comprises an amino acid sequences o SEQ ID NO:538. In some embodiments, the VH CDR2 comprises an amino acid sequence of PGX₁G (SEQ ID NO:778), wherein X₁ is D or S. In some embodiments, the VH CDR3 comprises an amino acid sequence of X₁GX₂YTX₃D (SEQ ID NO:779), wherein X₁ is Y or M, X₂ is D or M, and X₃ is I or L. In another aspect, provided herein is a TRGV9 antibody, comprising a VL comprising a VL CDR1, VL CDR2 and VL CDR3. In one embodiment, the VL CDR1 comprises an amino acid sequence of SQSX₁LYSSNX₂×₃ (SEQ ID NO:780), wherein X₁ is L or V, X₂ is Q or N, and X₃ is K or KNY. In some embodiments, the VL CDR2 comprises an amino acid sequence of SEQ ID NO:182. In some embodiments, the VL CDR2 comprises an amino acid sequence of SEQ ID NO:398. In some embodiments, the VL CDR2 comprises an amino acid sequence of SEQ ID NO:434. In some embodiments, the VL CDR2 comprises an amino acid sequence of SEQ ID NO:470. In some embodiments, the VL CDR3 comprises an amino acid sequence of SEQ ID NO:542. In some embodiments, the VL CDR3 comprises an amino acid sequence of SEQ ID NO:399. In some embodiments, the VL CDR3 comprises an amino acid sequence of SEQ ID NO:435. In some embodiments, the VL CDR3 comprises an amino acid sequence of SEQ ID NO:471. In some embodiments, the VL CDR3 comprises an amino acid sequence of SEQ ID NO:507. In some embodiments, the VL CDR3 comprises an amino acid sequence of SEQ ID NO:543.

In another aspect, provided is a multispecific TRGV9 antibody, comprising a TRGV9 antibody provided herein. In some embodiments, the multispecific TRGV9 antibody is a bispecific antibody. In one aspect, provided herein is a multispecific TRGV9 antibody, wherein the antibody comprises a VH CDR1, VH CDR2 and VH CDR3 of a TRGV9 antibody provided herein. In one aspect, provided herein is a multispecific TRGV9 antibody, wherein the antibody comprises a VL CDR1, VL CDR2 and VL CDR3 of a TRGV9 antibody provided herein. In one aspect, provided herein is a multispecific TRGV9 antibody, wherein the antibody comprises a VH CDR1, VH CDR2, VH CDR3, VL CDR1, VL CDR2 and VL CDR3 of a TRGV9 antibody provided herein. In one aspect, provided herein is a multispecific TRGV9 antibody, wherein the antibody comprises a VH of a TRGV9 antibody provided herein. In one aspect, provided herein is a multispecific TRGV9 antibody, wherein the antibody comprises a VL of a TRGV9 antibody provided herein. In one aspect, provided herein is a multispecific TRGV9 antibody, wherein the antibody comprises a VH and VL of a TRGV9 antibody provided herein.

In one aspect, provided herein is a multispecific TRGV9 antibody comprising: (a) a first binding domain that binds to TRGV9, and (b) a second binding domain that binds to a second target that is not TRGV9. In some embodiments of the multispecific TRGV9 antibodies provided herein, the second target is not a TRGV9 antigen. In some embodiments of the multispecific TRGV9 antibodies provided herein, the second target is not a TRGV9 epitope.

In one embodiment of a multispecific TRGV9 antibody provided herein, the first binding domain that binds to TRGV9 comprises a VH and VL amino acid sequence of L7A5_1 (TRGV9_1). In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:7. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:7; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In one embodiment, the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In one embodiment, the first binding domain comprises: (i) a VH comprising a VH CDR1 having an amino acid sequence of SEQ ID NO:160, a VH CDR2 having an amino acid sequence of SEQ ID NO:161, and a VH CDR3 having an amino acid sequence of SEQ ID NO:162; and (ii) a VL comprising a VL CDR1 having an amino acid sequence of SEQ ID NO:163, a VL CDR2 having an amino acid sequence of SEQ ID NO:164, and a VL CDR3 having an amino acid sequence of SEQ ID NO:165. In one embodiment, the first binding domain comprises: (i) a VH comprising a VH CDR1 having an amino acid sequence of SEQ ID NO:166, a VH CDR2 having an amino acid sequence of SEQ ID NO:167, and a VH CDR3 having an amino acid sequence of SEQ ID NO:168; and (ii) a VL comprising a VL CDR1 having an amino acid sequence of SEQ ID NO:169, a VL CDR2 having an amino acid sequence of SEQ ID NO:170, and a VL CDR3 having an amino acid sequence of SEQ ID NO:171. In one embodiment, the first binding domain comprises: (i) a VH comprising a VH CDR1 having an amino acid sequence of SEQ ID NO:172, a VH CDR2 having an amino acid sequence of SEQ ID NO:173, and a VH CDR3 having an amino acid sequence of SEQ ID NO:174; and (ii) a VL comprising a VL CDR1 having an amino acid sequence of SEQ ID NO:175, a VL CDR2 having an amino acid sequence of SEQ ID NO:176, and a VL CDR3 having an amino acid sequence of SEQ ID NO:177. In one embodiment, the first binding domain comprises: (i) a VH comprising a VH CDR1 having an amino acid sequence of SEQ ID NO:178, a VH CDR2 having an amino acid sequence of SEQ ID NO:179, and a VH CDR3 having an amino acid sequence of SEQ ID NO:180; and (ii) a VL comprising a VL CDR1 having an amino acid sequence of SEQ ID NO:181, a VL CDR2 having an amino acid sequence of SEQ ID NO:182, and a VL CDR3 having an amino acid sequence of SEQ ID NO:183. In one embodiment, the first binding domain comprises: (i) a VH comprising a VH CDR1 having an amino acid sequence of SEQ ID NO:178, a VH CDR2 having an amino acid sequence of SEQ ID NO:700, and a VH CDR3 having an amino acid sequence of SEQ ID NO:701; and (ii) a VL comprising a VL CDR1 having an amino acid sequence of SEQ ID NO:181, a VL CDR2 having an amino acid sequence of SEQ ID NO:182, and a VL CDR3 having an amino acid sequence of SEQ ID NO:183. In one embodiment, the first binding domain comprises: (i) a VH comprising a VH CDR1 having an amino acid sequence of SEQ ID NO:184, a VH CDR2 having an amino acid sequence of SEQ ID NO:185, and a VH CDR3 having an amino acid sequence of SEQ ID NO:186; and (ii) a VL comprising a VL CDR1 having an amino acid sequence of SEQ ID NO:187, a VL CDR2 having an amino acid sequence of SEQ ID NO:188, and a VL CDR3 having an amino acid sequence of SEQ ID NO:189. In one embodiment, the first binding domain comprises: (i) a VH comprising a VH CDR1 having an amino acid sequence of SEQ ID NO:190, a VH CDR2 having an amino acid sequence of SEQ ID NO:191, and a VH CDR3 having an amino acid sequence of SEQ ID NO:192; and (ii) a VL comprising a VL CDR1 having an amino acid sequence of SEQ ID NO:193, a VL CDR2 having an amino acid sequence of SEQ ID NO:194, and a VL CDR3 having an amino acid sequence of SEQ ID NO:195. In some embodiments, the first binding domain comprises a VH having an amino acid sequence of SEQ ID NO:7. In some embodiments, the first binding domain comprises a VL having an amino acid sequence of SEQ ID NO:8. In some embodiments, the first binding domain comprises a VH having an amino acid sequence of SEQ ID NO:7, and a VL having an amino acid sequence of SEQ ID NO:8. In some embodiments, the first binding domain comprises a heavy chain having an amino acid sequence of SEQ ID NO:23. In some embodiments, the first binding domain comprises a light chain having an amino acid sequence of SEQ ID NO:24. In some embodiments, the first binding domain comprises a heavy chain having an amino acid sequence of SEQ ID NO:23, and a light chain having an amino acid sequence of SEQ ID NO:24. In some embodiments, the first binding domain comprises an amino acid sequence of SEQ ID NO:17. In some embodiments, the first binding domain comprises a heavy chain having an amino acid sequence of SEQ ID NO:69. In some embodiments, the first binding domain comprises a light chain having an amino acid sequence of SEQ ID NO:24. In some embodiments, the first binding domain comprises a heavy chain having an amino acid sequence of SEQ ID NO:69, and a light chain having an amino acid sequence of SEQ ID NO:24. In some embodiments, the first binding domain comprises a VH comprising an amino acid sequence having at least 95% identity to the amino acid sequence of SEQ ID NO:7. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:7, and a VL comprising an amino acid sequence having at least 95% identity to the amino acid sequence of SEQ ID NO:8. In some embodiments, the first binding domain comprises a heavy chain comprising an amino acid sequence having at least 95% identity to the amino acid sequence of SEQ ID NO:23. In some embodiments, the first binding domain comprises a light chain comprising an amino acid sequence having at least 95% identity to the amino acid sequence of SEQ ID NO:24. In some embodiments, the first binding domain comprises a heavy chain comprising an amino acid sequence having at least 95% identity to the amino acid sequence of SEQ ID NO:23, and a light chain comprising an amino acid sequence having at least 95% identity to the amino acid sequence of SEQ ID NO:24. In some embodiments, the first binding domain comprises an amino acid sequence of SEQ ID NO:17. In some embodiments, the first binding domain comprises a heavy chain comprising an amino acid sequence having at least 95% identity to the amino acid sequence of SEQ ID NO:69. In some embodiments, the first binding domain comprises a light chain comprising an amino acid sequence having at least 95% identity to the amino acid sequence of SEQ ID NO:24. In some embodiments, the first binding domain comprises a heavy chain comprising an amino acid sequence having at least 95% identity to the amino acid sequence of SEQ ID NO:69, and a light chain comprising an amino acid sequence having at least 95% identity to the amino acid sequence of SEQ ID NO:24.

In one embodiment of a multispecific TRGV9 antibody provided herein, the first binding domain that binds to TRGV9 comprises a VH and VL amino acid sequence of L7A5_2 (TRGV9_2). In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:34. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:34; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In one embodiment, the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:31; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In one embodiment, the first binding domain comprises: (i) a VH comprising a VH CDR1 having an amino acid sequence of SEQ ID NO:196, a VH CDR2 having an amino acid sequence of SEQ ID NO:197, and a VH CDR3 having an amino acid sequence of SEQ ID NO:198; and (ii) a VL comprising a VL CDR1 having an amino acid sequence of SEQ ID NO:199, a VL CDR2 having an amino acid sequence of SEQ ID NO:200, and a VL CDR3 having an amino acid sequence of SEQ ID NO:201. In one embodiment, the first binding domain comprises: (i) a VH comprising a VH CDR1 having an amino acid sequence of SEQ ID NO:202, a VH CDR2 having an amino acid sequence of SEQ ID NO:203, and a VH CDR3 having an amino acid sequence of SEQ ID NO:204; and (ii) a VL comprising a VL CDR1 having an amino acid sequence of SEQ ID NO:205, a VL CDR2 having an amino acid sequence of SEQ ID NO:206, and a VL CDR3 having an amino acid sequence of SEQ ID NO:207. In one embodiment, the first binding domain comprises: (i) a VH comprising a VH CDR1 having an amino acid sequence of SEQ ID NO:208, a VH CDR2 having an amino acid sequence of SEQ ID NO:209, and a VH CDR3 having an amino acid sequence of SEQ ID NO:210; and (ii) a VL comprising a VL CDR1 having an amino acid sequence of SEQ ID NO:211, a VL CDR2 having an amino acid sequence of SEQ ID NO:212, and a VL CDR3 having an amino acid sequence of SEQ ID NO:213. In one embodiment, the first binding domain comprises: (i) a VH comprising a VH CDR1 having an amino acid sequence of SEQ ID NO:214, a VH CDR2 having an amino acid sequence of SEQ ID NO:215, and a VH CDR3 having an amino acid sequence of SEQ ID NO:216; and (ii) a VL comprising a VL CDR1 having an amino acid sequence of SEQ ID NO:217, a VL CDR2 having an amino acid sequence of SEQ ID NO:218, and a VL CDR3 having an amino acid sequence of SEQ ID NO:219. In one embodiment, the first binding domain comprises: (i) a VH comprising a VH CDR1 having an amino acid sequence of SEQ ID NO:214, a VH CDR2 having an amino acid sequence of SEQ ID NO:702, and a VH CDR3 having an amino acid sequence of SEQ ID NO:703; and (ii) a VL comprising a VL CDR1 having an amino acid sequence of SEQ ID NO:217, a VL CDR2 having an amino acid sequence of SEQ ID NO:218, and a VL CDR3 having an amino acid sequence of SEQ ID NO:219. In one embodiment, the first binding domain comprises: (i) a VH comprising a VH CDR1 having an amino acid sequence of SEQ ID NO:220, a VH CDR2 having an amino acid sequence of SEQ ID NO:221, and a VH CDR3 having an amino acid sequence of SEQ ID NO:222; and (ii) a VL comprising a VL CDR1 having an amino acid sequence of SEQ ID NO:223, a VL CDR2 having an amino acid sequence of SEQ ID NO:224, and a VL CDR3 having an amino acid sequence of SEQ ID NO:225. In one embodiment, the first binding domain comprises: (i) a VH comprising a VH CDR1 having an amino acid sequence of SEQ ID NO:226, a VH CDR2 having an amino acid sequence of SEQ ID NO:227, and a VH CDR3 having an amino acid sequence of SEQ ID NO:228; and (ii) a VL comprising a VL CDR1 having an amino acid sequence of SEQ ID NO:229, a VL CDR2 having an amino acid sequence of SEQ ID NO:230, and a VL CDR3 having an amino acid sequence of SEQ ID NO:231. In some embodiments, the first binding domain comprises a VH having an amino acid sequence of SEQ ID NO:34. In some embodiments, the first binding domain comprises a VL having an amino acid sequence of SEQ ID NO:8. In some embodiments, the first binding domain comprises a VH having an amino acid sequence of SEQ ID NO:34, and a VL having an amino acid sequence of SEQ ID NO:8. In some embodiments, the first binding domain comprises a VH comprising an amino acid sequence having at least 95% identity to the amino acid sequence of SEQ ID NO:34. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:34, and a VL comprising an amino acid sequence having at least 95% identity to the amino acid sequence of SEQ ID NO:8.

In one embodiment of a multispecific TRGV9 antibody provided herein, the first binding domain that binds to TRGV9 comprises a VH and VL amino acid sequence of L7A5_3 (TRGV9_3). In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:35. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:35; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In one embodiment, the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:32; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In one embodiment, the first binding domain comprises: (i) a VH comprising a VH CDR1 having an amino acid sequence of SEQ ID NO:232, a VH CDR2 having an amino acid sequence of SEQ ID NO:233, and a VH CDR3 having an amino acid sequence of SEQ ID NO:234; and (ii) a VL comprising a VL CDR1 having an amino acid sequence of SEQ ID NO:235, a VL CDR2 having an amino acid sequence of SEQ ID NO:236, and a VL CDR3 having an amino acid sequence of SEQ ID NO:237. In one embodiment, the first binding domain comprises: (i) a VH comprising a VH CDR1 having an amino acid sequence of SEQ ID NO:238, a VH CDR2 having an amino acid sequence of SEQ ID NO:239, and a VH CDR3 having an amino acid sequence of SEQ ID NO:240; and (ii) a VL comprising a VL CDR1 having an amino acid sequence of SEQ ID NO:241, a VL CDR2 having an amino acid sequence of SEQ ID NO:242, and a VL CDR3 having an amino acid sequence of SEQ ID NO:243. In one embodiment, the first binding domain comprises: (i) a VH comprising a VH CDR1 having an amino acid sequence of SEQ ID NO:244, a VH CDR2 having an amino acid sequence of SEQ ID NO:245, and a VH CDR3 having an amino acid sequence of SEQ ID NO:246; and (ii) a VL comprising a VL CDR1 having an amino acid sequence of SEQ ID NO:247, a VL CDR2 having an amino acid sequence of SEQ ID NO:248, and a VL CDR3 having an amino acid sequence of SEQ ID NO:249. In one embodiment, the first binding domain comprises: (i) a VH comprising a VH CDR1 having an amino acid sequence of SEQ ID NO:250, a VH CDR2 having an amino acid sequence of SEQ ID NO:251, and a VH CDR3 having an amino acid sequence of SEQ ID NO:252; and (ii) a VL comprising a VL CDR1 having an amino acid sequence of SEQ ID NO:253, a VL CDR2 having an amino acid sequence of SEQ ID NO:254, and a VL CDR3 having an amino acid sequence of SEQ ID NO:255. In one embodiment, the first binding domain comprises: (i) a VH comprising a VH CDR1 having an amino acid sequence of SEQ ID NO:250, a VH CDR2 having an amino acid sequence of SEQ ID NO:704, and a VH CDR3 having an amino acid sequence of SEQ ID NO:705; and (ii) a VL comprising a VL CDR1 having an amino acid sequence of SEQ ID NO:253, a VL CDR2 having an amino acid sequence of SEQ ID NO:254, and a VL CDR3 having an amino acid sequence of SEQ ID NO:255. In one embodiment, the first binding domain comprises: (i) a VH comprising a VH CDR1 having an amino acid sequence of SEQ ID NO:256, a VH CDR2 having an amino acid sequence of SEQ ID NO:257, and a VH CDR3 having an amino acid sequence of SEQ ID NO:258; and (ii) a VL comprising a VL CDR1 having an amino acid sequence of SEQ ID NO:259, a VL CDR2 having an amino acid sequence of SEQ ID NO:260, and a VL CDR3 having an amino acid sequence of SEQ ID NO:261. In one embodiment, the first binding domain comprises: (i) a VH comprising a VH CDR1 having an amino acid sequence of SEQ ID NO:262, a VH CDR2 having an amino acid sequence of SEQ ID NO:263, and a VH CDR3 having an amino acid sequence of SEQ ID NO:264; and (ii) a VL comprising a VL CDR1 having an amino acid sequence of SEQ ID NO:265, a VL CDR2 having an amino acid sequence of SEQ ID NO:266, and a VL CDR3 having an amino acid sequence of SEQ ID NO:267. In some embodiments, the first binding domain comprises a VH having an amino acid sequence of SEQ ID NO:35. In some embodiments, the first binding domain comprises a VL having an amino acid sequence of SEQ ID NO:8. In some embodiments, the first binding domain comprises a VH having an amino acid sequence of SEQ ID NO:35, and a VL having an amino acid sequence of SEQ ID NO:8. In some embodiments, the first binding domain comprises a VH comprising an amino acid sequence having at least 95% identity to the amino acid sequence of SEQ ID NO:35. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:35, and a VL comprising an amino acid sequence having at least 95% identity to the amino acid sequence of SEQ ID NO:8.

In one embodiment of a multispecific TRGV9 antibody provided herein, the first binding domain that binds to TRGV9 comprises a VH and VL amino acid sequence of L7A5_4 (TRGV9_4). In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:36. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:36; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8. In one embodiment, the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:33; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In one embodiment, the first binding domain comprises: (i) a VH comprising a VH CDR1 having an amino acid sequence of SEQ ID NO:268, a VH CDR2 having an amino acid sequence of SEQ ID NO:269, and a VH CDR3 having an amino acid sequence of SEQ ID NO:270; and (ii) a VL comprising a VL CDR1 having an amino acid sequence of SEQ ID NO:271, a VL CDR2 having an amino acid sequence of SEQ ID NO:272, and a VL CDR3 having an amino acid sequence of SEQ ID NO:273. In one embodiment, the first binding domain comprises: (i) a VH comprising a VH CDR1 having an amino acid sequence of SEQ ID NO:274, a VH CDR2 having an amino acid sequence of SEQ ID NO:275, and a VH CDR3 having an amino acid sequence of SEQ ID NO:276; and (ii) a VL comprising a VL CDR1 having an amino acid sequence of SEQ ID NO:277, a VL CDR2 having an amino acid sequence of SEQ ID NO:278, and a VL CDR3 having an amino acid sequence of SEQ ID NO:279. In one embodiment, the first binding domain comprises: (i) a VH comprising a VH CDR1 having an amino acid sequence of SEQ ID NO:280, a VH CDR2 having an amino acid sequence of SEQ ID NO:281, and a VH CDR3 having an amino acid sequence of SEQ ID NO:282; and (ii) a VL comprising a VL CDR1 having an amino acid sequence of SEQ ID NO:283, a VL CDR2 having an amino acid sequence of SEQ ID NO:284, and a VL CDR3 having an amino acid sequence of SEQ ID NO:285. In one embodiment, the first binding domain comprises: (i) a VH comprising a VH CDR1 having an amino acid sequence of SEQ ID NO:286, a VH CDR2 having an amino acid sequence of SEQ ID NO:287, and a VH CDR3 having an amino acid sequence of SEQ ID NO:288; and (ii) a VL comprising a VL CDR1 having an amino acid sequence of SEQ ID NO:289, a VL CDR2 having an amino acid sequence of SEQ ID NO:290, and a VL CDR3 having an amino acid sequence of SEQ ID NO:291. In one embodiment, the first binding domain comprises: (i) a VH comprising a VH CDR1 having an amino acid sequence of SEQ ID NO:286, a VH CDR2 having an amino acid sequence of SEQ ID NO:706, and a VH CDR3 having an amino acid sequence of SEQ ID NO:707; and (ii) a VL comprising a VL CDR1 having an amino acid sequence of SEQ ID NO:289, a VL CDR2 having an amino acid sequence of SEQ ID NO:290, and a VL CDR3 having an amino acid sequence of SEQ ID NO:291. In one embodiment, the first binding domain comprises: (i) a VH comprising a VH CDR1 having an amino acid sequence of SEQ ID NO:292, a VH CDR2 having an amino acid sequence of SEQ ID NO:293, and a VH CDR3 having an amino acid sequence of SEQ ID NO:294; and (ii) a VL comprising a VL CDR1 having an amino acid sequence of SEQ ID NO:295, a VL CDR2 having an amino acid sequence of SEQ ID NO:296, and a VL CDR3 having an amino acid sequence of SEQ ID NO:297. In one embodiment, the first binding domain comprises: (i) a VH comprising a VH CDR1 having an amino acid sequence of SEQ ID NO:298, a VH CDR2 having an amino acid sequence of SEQ ID NO:299, and a VH CDR3 having an amino acid sequence of SEQ ID NO:300; and (ii) a VL comprising a VL CDR1 having an amino acid sequence of SEQ ID NO:301, a VL CDR2 having an amino acid sequence of SEQ ID NO:302, and a VL CDR3 having an amino acid sequence of SEQ ID NO:303. In some embodiments, the first binding domain comprises a VH having an amino acid sequence of SEQ ID NO:36. In some embodiments, the first binding domain comprises a VL having an amino acid sequence of SEQ ID NO:8. In some embodiments, the first binding domain comprises a VH having an amino acid sequence of SEQ ID NO:36, and a VL having an amino acid sequence of SEQ ID NO:8. In some embodiments, the first binding domain comprises a VH comprising an amino acid sequence having at least 95% identity to the amino acid sequence of SEQ ID NO:36. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:36, and a VL comprising an amino acid sequence having at least 95% identity to the amino acid sequence of SEQ ID NO:8.

In one embodiment of a multispecific TRGV9 antibody provided herein, the first binding domain that binds to TRGV9 comprises a VH and VL amino acid sequence of TRGV9Ab_var17. In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment of a multispecific TRGV9 antibody provided herein, the first binding domain comprises a VH and VL amino acid sequence of TRGV9Ab_var29. In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68. In one embodiment, the first binding domain comprises: (i) a VH comprising a VH CDR1 having an amino acid sequence of SEQ ID NO:1, a VH CDR2 having an amino acid sequence of SEQ ID NO:76, and a VH CDR3 having an amino acid sequence of SEQ ID NO:3; and (ii) a VL comprising a VL CDR1 having an amino acid sequence of SEQ ID NO:77, a VL CDR2 having an amino acid sequence of SEQ ID NO:5, and a VL CDR3 having an amino acid sequence of SEQ ID NO:6. In one embodiment, the first binding domain comprises: (i) a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and (ii) a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6. In one embodiment, the first binding domain comprises: (i) a VH comprising a VH CDR1 having an amino acid sequence of SEQ ID NO:304, a VH CDR2 having an amino acid sequence of SEQ ID NO:305, and a VH CDR3 having an amino acid sequence of SEQ ID NO:306; and (ii) a VL comprising a VL CDR1 having an amino acid sequence of SEQ ID NO:307, a VL CDR2 having an amino acid sequence of SEQ ID NO:308, and a VL CDR3 having an amino acid sequence of SEQ ID NO:309. In one embodiment, the first binding domain comprises: (i) a VH comprising a VH CDR1 having an amino acid sequence of SEQ ID NO:310, a VH CDR2 having an amino acid sequence of SEQ ID NO:311, and a VH CDR3 having an amino acid sequence of SEQ ID NO:312; and (ii) a VL comprising a VL CDR1 having an amino acid sequence of SEQ ID NO:313, a VL CDR2 having an amino acid sequence of SEQ ID NO:314, and a VL CDR3 having an amino acid sequence of SEQ ID NO:315. In one embodiment, the first binding domain comprises: (i) a VH comprising a VH CDR1 having an amino acid sequence of SEQ ID NO:316, a VH CDR2 having an amino acid sequence of SEQ ID NO:317, and a VH CDR3 having an amino acid sequence of SEQ ID NO:318; and (ii) a VL comprising a VL CDR1 having an amino acid sequence of SEQ ID NO:319, a VL CDR2 having an amino acid sequence of SEQ ID NO:320, and a VL CDR3 having an amino acid sequence of SEQ ID NO:321. In one embodiment, the first binding domain comprises: (i) a VH comprising a VH CDR1 having an amino acid sequence of SEQ ID NO:322, a VH CDR2 having an amino acid sequence of SEQ ID NO:323, and a VH CDR3 having an amino acid sequence of SEQ ID NO:324; and (ii) a VL comprising a VL CDR1 having an amino acid sequence of SEQ ID NO:325, a VL CDR2 having an amino acid sequence of SEQ ID NO:326, and a VL CDR3 having an amino acid sequence of SEQ ID NO:327. In one embodiment, the first binding domain comprises: (i) a VH comprising a VH CDR1 having an amino acid sequence of SEQ ID NO:322, a VH CDR2 having an amino acid sequence of SEQ ID NO:708, and a VH CDR3 having an amino acid sequence of SEQ ID NO:709; and (ii) a VL comprising a VL CDR1 having an amino acid sequence of SEQ ID NO:325, a VL CDR2 having an amino acid sequence of SEQ ID NO:326, and a VL CDR3 having an amino acid sequence of SEQ ID NO:327. In one embodiment, the first binding domain comprises: (i) a VH comprising a VH CDR1 having an amino acid sequence of SEQ ID NO:328, a VH CDR2 having an amino acid sequence of SEQ ID NO:329, and a VH CDR3 having an amino acid sequence of SEQ ID NO:330; and (ii) a VL comprising a VL CDR1 having an amino acid sequence of SEQ ID NO:331, a VL CDR2 having an amino acid sequence of SEQ ID NO:332, and a VL CDR3 having an amino acid sequence of SEQ ID NO:333. In one embodiment, the first binding domain comprises: (i) a VH comprising a VH CDR1 having an amino acid sequence of SEQ ID NO:334, a VH CDR2 having an amino acid sequence of SEQ ID NO:335, and a VH CDR3 having an amino acid sequence of SEQ ID NO:336; and (ii) a VL comprising a VL CDR1 having an amino acid sequence of SEQ ID NO:337, a VL CDR2 having an amino acid sequence of SEQ ID NO:338, and a VL CDR3 having an amino acid sequence of SEQ ID NO:339. In one embodiment, the first binding domain comprises: (i) a VH comprising a VH CDR1 having an amino acid sequence of SEQ ID NO:340, a VH CDR2 having an amino acid sequence of SEQ ID NO:341, and a VH CDR3 having an amino acid sequence of SEQ ID NO:342; and (ii) a VL comprising a VL CDR1 having an amino acid sequence of SEQ ID NO:343, a VL CDR2 having an amino acid sequence of SEQ ID NO:344, and a VL CDR3 having an amino acid sequence of SEQ ID NO:345. In one embodiment, the first binding domain comprises: (i) a VH comprising a VH CDR1 having an amino acid sequence of SEQ ID NO:346, a VH CDR2 having an amino acid sequence of SEQ ID NO:347, and a VH CDR3 having an amino acid sequence of SEQ ID NO:348; and (ii) a VL comprising a VL CDR1 having an amino acid sequence of SEQ ID NO:349, a VL CDR2 having an amino acid sequence of SEQ ID NO:350, and a VL CDR3 having an amino acid sequence of SEQ ID NO:351. In one embodiment, the first binding domain comprises: (i) a VH comprising a VH CDR1 having an amino acid sequence of SEQ ID NO:352, a VH CDR2 having an amino acid sequence of SEQ ID NO:353, and a VH CDR3 having an amino acid sequence of SEQ ID NO:354; and (ii) a VL comprising a VL CDR1 having an amino acid sequence of SEQ ID NO:355, a VL CDR2 having an amino acid sequence of SEQ ID NO:356, and a VL CDR3 having an amino acid sequence of SEQ ID NO:357. In one embodiment, the first binding domain comprises: (i) a VH comprising a VH CDR1 having an amino acid sequence of SEQ ID NO:358, a VH CDR2 having an amino acid sequence of SEQ ID NO:359, and a VH CDR3 having an amino acid sequence of SEQ ID NO:360; and (ii) a VL comprising a VL CDR1 having an amino acid sequence of SEQ ID NO:361, a VL CDR2 having an amino acid sequence of SEQ ID NO:362, and a VL CDR3 having an amino acid sequence of SEQ ID NO:363. In one embodiment, the first binding domain comprises: (i) a VH comprising a VH CDR1 having an amino acid sequence of SEQ ID NO:358, a VH CDR2 having an amino acid sequence of SEQ ID NO:710, and a VH CDR3 having an amino acid sequence of SEQ ID NO:711; and (ii) a VL comprising a VL CDR1 having an amino acid sequence of SEQ ID NO:361, a VL CDR2 having an amino acid sequence of SEQ ID NO:362, and a VL CDR3 having an amino acid sequence of SEQ ID NO:363. In one embodiment, the first binding domain comprises: (i) a VH comprising a VH CDR1 having an amino acid sequence of SEQ ID NO:364, a VH CDR2 having an amino acid sequence of SEQ ID NO:365, and a VH CDR3 having an amino acid sequence of SEQ ID NO:366; and (ii) a VL comprising a VL CDR1 having an amino acid sequence of SEQ ID NO:367, a VL CDR2 having an amino acid sequence of SEQ ID NO:368, and a VL CDR3 having an amino acid sequence of SEQ ID NO:369. In one embodiment, the first binding domain comprises: (i) a VH comprising a VH CDR1 having an amino acid sequence of SEQ ID NO:370, a VH CDR2 having an amino acid sequence of SEQ ID NO:371, and a VH CDR3 having an amino acid sequence of SEQ ID NO:372; and (ii) a VL comprising a VL CDR1 having an amino acid sequence of SEQ ID NO:373, a VL CDR2 having an amino acid sequence of SEQ ID NO:374, and a VL CDR3 having an amino acid sequence of SEQ ID NO:375. In some embodiments, the first binding domain comprises a VH having an amino acid sequence of SEQ ID NO:65. In some embodiments, the first binding domain comprises a VL having an amino acid sequence of SEQ ID NO:66. In some embodiments, the first binding domain comprises a VH having an amino acid sequence of SEQ ID NO:65, and a VL having an amino acid sequence of SEQ ID NO:66. In some embodiments, the first binding domain comprises a VH having an amino acid sequence of SEQ ID NO:67. In some embodiments, the first binding domain comprises a VL having an amino acid sequence of SEQ ID NO:68. In some embodiments, the first binding domain a VH having an amino acid sequence of SEQ ID NO:67, and a VL having an amino acid sequence of SEQ ID NO:68. In some embodiments, the first binding domain comprises a heavy chain having an amino acid sequence of SEQ ID NO:71. In some embodiments, the first binding domain comprises a light chain having an amino acid sequence of SEQ ID NO:72. In some embodiments, the first binding domain comprises a heavy chain having an amino acid sequence of SEQ ID NO:71, and a light chain having an amino acid sequence of SEQ ID NO:72. In some embodiments, the first binding domain comprises an amino acid sequence of SEQ ID NO:70. In some embodiments, the first binding domain comprises a heavy chain having an amino acid sequence of SEQ ID NO:74. In some embodiments, the first binding domain comprises a light chain having an amino acid sequence of SEQ ID NO:75. In some embodiments, the first binding domain comprises a heavy chain having an amino acid sequence of SEQ ID NO:74, and a light chain having an amino acid sequence of SEQ ID NO:75. In some embodiments, the first binding domain comprises an amino acid sequence of SEQ ID NO:73. In some embodiments, the first binding domain comprises a VH comprising an amino acid sequence having at least 95% identity to the amino acid sequence of SEQ ID NO:65. In some embodiments, the first binding domain comprises a VL comprising an amino acid sequence having at least 95% identity to the amino acid sequence of SEQ ID NO:66. In some embodiments, the first binding domain comprises a VH comprising an amino acid sequence having at least 95% identity to the amino acid sequence of SEQ ID NO:65, and a VL comprising an amino acid sequence having at least 95% identity to the amino acid sequence of SEQ ID NO:66. In some embodiments, the first binding domain comprises a VH comprising an amino acid sequence having at least 95% identity to the amino acid sequence of SEQ ID NO:67. In some embodiments, the first binding domain comprises a VL comprising an amino acid sequence having at least 95% identity to the amino acid sequence of SEQ ID NO:68. In some embodiments, the first binding domain a VH comprising an amino acid sequence having at least 95% identity to the amino acid sequence of SEQ ID NO:67, and a VL comprising an amino acid sequence having at least 95% identity to the amino acid sequence of SEQ ID NO:68. In some embodiments, the first binding domain comprises a heavy chain comprising an amino acid sequence having at least 95% identity to the amino acid sequence of SEQ ID NO:71. In some embodiments, the first binding domain comprises a light chain comprising an amino acid sequence having at least 95% identity to the amino acid sequence of SEQ ID NO:72. In some embodiments, the first binding domain comprises a heavy chain comprising an amino acid sequence having at least 95% identity to the amino acid sequence of SEQ ID NO:71, and a light chain comprising an amino acid sequence having at least 95% identity to the amino acid sequence of SEQ ID NO:72. In some embodiments, the first binding domain comprises an amino acid sequence having at least 95% identity to an amino acid sequence of SEQ ID NO:70. In some embodiments, the first binding domain comprises a heavy chain comprising an amino acid sequence having at least 95% identity to the amino acid sequence of SEQ ID NO:74. In some embodiments, the first binding domain comprises a light chain comprising an amino acid sequence having at least 95% identity to the amino acid sequence of SEQ ID NO:75. In some embodiments, the first binding domain comprises a heavy chain comprising an amino acid sequence having at least 95% identity to the amino acid sequence of SEQ ID NO:74, and a light chain comprising an amino acid sequence having at least 95% identity to the amino acid sequence of SEQ ID NO:75. In some embodiments, the first binding domain comprises an amino acid sequence having at least 95% identity to an amino acid sequence of SEQ ID NO:73.

In one embodiment of a multispecific TRGV9 antibody provided herein, the first binding domain that binds to TRGV9 comprises a VH and VL amino acid sequence of VG9_B3_RN. In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:95. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96. In one embodiment, the first binding domain comprises: (i) a VH comprising a VH CDR1 having an amino acid sequence of SEQ ID NO:89, a VH CDR2 having an amino acid sequence of SEQ ID NO:90, and a VH CDR3 having an amino acid sequence of SEQ ID NO:91; and (ii) a VL comprising a VL CDR1 having an amino acid sequence of SEQ ID NO:92, a VL CDR2 having an amino acid sequence of SEQ ID NO:93, and a VL CDR3 having an amino acid sequence of SEQ ID NO:94. In one embodiment, the first binding domain comprises: (i) a VH comprising a VH CDR1 having an amino acid sequence of SEQ ID NO:376, a VH CDR2 having an amino acid sequence of SEQ ID NO:377, and a VH CDR3 having an amino acid sequence of SEQ ID NO:378; and (ii) a VL comprising a VL CDR1 having an amino acid sequence of SEQ ID NO:379, a VL CDR2 having an amino acid sequence of SEQ ID NO:380, and a VL CDR3 having an amino acid sequence of SEQ ID NO:381. In one embodiment, the first binding domain comprises: (i) a VH comprising a VH CDR1 having an amino acid sequence of SEQ ID NO:382, a VH CDR2 having an amino acid sequence of SEQ ID NO:383, and a VH CDR3 having an amino acid sequence of SEQ ID NO:384; and (ii) a VL comprising a VL CDR1 having an amino acid sequence of SEQ ID NO:385, a VL CDR2 having an amino acid sequence of SEQ ID NO:386, and a VL CDR3 having an amino acid sequence of SEQ ID NO:387. In one embodiment, the first binding domain comprises: (i) a VH comprising a VH CDR1 having an amino acid sequence of SEQ ID NO:388, a VH CDR2 having an amino acid sequence of SEQ ID NO:389, and a VH CDR3 having an amino acid sequence of SEQ ID NO:390; and (ii) a VL comprising a VL CDR1 having an amino acid sequence of SEQ ID NO:391, a VL CDR2 having an amino acid sequence of SEQ ID NO:392, and a VL CDR3 having an amino acid sequence of SEQ ID NO:393. In one embodiment, the first binding domain comprises: (i) a VH comprising a VH CDR1 having an amino acid sequence of SEQ ID NO:394, a VH CDR2 having an amino acid sequence of SEQ ID NO:395, and a VH CDR3 having an amino acid sequence of SEQ ID NO:396; and (ii) a VL comprising a VL CDR1 having an amino acid sequence of SEQ ID NO:397, a VL CDR2 having an amino acid sequence of SEQ ID NO:398, and a VL CDR3 having an amino acid sequence of SEQ ID NO:399. In one embodiment, the first binding domain comprises: (i) a VH comprising a VH CDR1 having an amino acid sequence of SEQ ID NO:394, a VH CDR2 having an amino acid sequence of SEQ ID NO:712, and a VH CDR3 having an amino acid sequence of SEQ ID NO:713; and (ii) a VL comprising a VL CDR1 having an amino acid sequence of SEQ ID NO:397, a VL CDR2 having an amino acid sequence of SEQ ID NO:398, and a VL CDR3 having an amino acid sequence of SEQ ID NO:399. In one embodiment, the first binding domain comprises: (i) a VH comprising a VH CDR1 having an amino acid sequence of SEQ ID NO:400, a VH CDR2 having an amino acid sequence of SEQ ID NO:401, and a VH CDR3 having an amino acid sequence of SEQ ID NO:402; and (ii) a VL comprising a VL CDR1 having an amino acid sequence of SEQ ID NO:403, a VL CDR2 having an amino acid sequence of SEQ ID NO:404, and a VL CDR3 having an amino acid sequence of SEQ ID NO:405. In one embodiment, the first binding domain comprises: (i) a VH comprising a VH CDR1 having an amino acid sequence of SEQ ID NO:406, a VH CDR2 having an amino acid sequence of SEQ ID NO:407, and a VH CDR3 having an amino acid sequence of SEQ ID NO:408; and (ii) a VL comprising a VL CDR1 having an amino acid sequence of SEQ ID NO:409, a VL CDR2 having an amino acid sequence of SEQ ID NO:410, and a VL CDR3 having an amino acid sequence of SEQ ID NO:411. In some embodiments, the first binding domain comprises a VH having an amino acid sequence of SEQ ID NO:95. In some embodiments, the first binding domain comprises a VL having an amino acid sequence of SEQ ID NO:96. In some embodiments, the first binding domain comprises a VH having an amino acid sequence of SEQ ID NO:95, and a VL having an amino acid sequence of SEQ ID NO:96. In some embodiments, the first binding domain comprises an amino acid sequence of SEQ ID NO:97. In some embodiments, the first binding domain comprises a VH comprising an amino acid sequence having at least 95% identity to the amino acid sequence of SEQ ID NO:95. In some embodiments, the first binding domain comprises a VL comprising an amino acid sequence having at least 95% identity to the amino acid sequence of SEQ ID NO:96. In some embodiments, the first binding domain comprises a VH comprising an amino acid sequence having at least 95% identity to the amino acid sequence of SEQ ID NO:95, and a VL comprising an amino acid sequence having at least 95% identity to the amino acid sequence of SEQ ID NO:96. In some embodiments, the first binding domain comprises an amino acid sequence having at least 95% identity to an amino acid sequence of SEQ ID NO:97.

In one embodiment of a multispecific TRGV9 antibody provided herein, the first binding domain that binds to TRGV9 comprises a VH and VL amino acid sequence of VG9B420. In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:104. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:105. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:104; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:105. In one embodiment, the first binding domain comprises: (i) a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100, and (ii) a VL comprising a VL CDR1 having an amino acid sequence of SEQ ID NO:101, a VL CDR2 having an amino acid sequence of SEQ ID NO:102, and a VL CDR3 having an amino acid sequence of SEQ ID NO:103. In one embodiment, the first binding domain comprises: (i) a VH comprising a VH CDR1 having an amino acid sequence of SEQ ID NO:412, a VH CDR2 having an amino acid sequence of SEQ ID NO:413, and a VH CDR3 having an amino acid sequence of SEQ ID NO:414; and (ii) a VL comprising a VL CDR1 having an amino acid sequence of SEQ ID NO:415, a VL CDR2 having an amino acid sequence of SEQ ID NO:416, and a VL CDR3 having an amino acid sequence of SEQ ID NO:417. In one embodiment, the first binding domain comprises: (i) a VH comprising a VH CDR1 having an amino acid sequence of SEQ ID NO:418, a VH CDR2 having an amino acid sequence of SEQ ID NO:419, and a VH CDR3 having an amino acid sequence of SEQ ID NO:420; and (ii) a VL comprising a VL CDR1 having an amino acid sequence of SEQ ID NO:421, a VL CDR2 having an amino acid sequence of SEQ ID NO:422, and a VL CDR3 having an amino acid sequence of SEQ ID NO:423. In one embodiment, the first binding domain comprises: (i) a VH comprising a VH CDR1 having an amino acid sequence of SEQ ID NO:424, a VH CDR2 having an amino acid sequence of SEQ ID NO:425, and a VH CDR3 having an amino acid sequence of SEQ ID NO:426; and (ii) a VL comprising a VL CDR1 having an amino acid sequence of SEQ ID NO:427, a VL CDR2 having an amino acid sequence of SEQ ID NO:428, and a VL CDR3 having an amino acid sequence of SEQ ID NO:429. In one embodiment, the first binding domain comprises: (i) a VH comprising a VH CDR1 having an amino acid sequence of SEQ ID NO:430, a VH CDR2 having an amino acid sequence of SEQ ID NO:431, and a VH CDR3 having an amino acid sequence of SEQ ID NO:432; and (ii) a VL comprising a VL CDR1 having an amino acid sequence of SEQ ID NO:433, a VL CDR2 having an amino acid sequence of SEQ ID NO:434, and a VL CDR3 having an amino acid sequence of SEQ ID NO:435. In one embodiment, the first binding domain comprises: (i) a VH comprising a VH CDR1 having an amino acid sequence of SEQ ID NO:430, a VH CDR2 having an amino acid sequence of SEQ ID NO:714, and a VH CDR3 having an amino acid sequence of SEQ ID NO:715; and (ii) a VL comprising a VL CDR1 having an amino acid sequence of SEQ ID NO:433, a VL CDR2 having an amino acid sequence of SEQ ID NO:434, and a VL CDR3 having an amino acid sequence of SEQ ID NO:435. In one embodiment, the first binding domain comprises: (i) a VH comprising a VH CDR1 having an amino acid sequence of SEQ ID NO:436, a VH CDR2 having an amino acid sequence of SEQ ID NO:437, and a VH CDR3 having an amino acid sequence of SEQ ID NO:438; and (ii) a VL comprising a VL CDR1 having an amino acid sequence of SEQ ID NO:439, a VL CDR2 having an amino acid sequence of SEQ ID NO:440, and a VL CDR3 having an amino acid sequence of SEQ ID NO:441. In one embodiment, the first binding domain comprises: (i) a VH comprising a VH CDR1 having an amino acid sequence of SEQ ID NO:442, a VH CDR2 having an amino acid sequence of SEQ ID NO:443, and a VH CDR3 having an amino acid sequence of SEQ ID NO:444; and (ii) a VL comprising a VL CDR1 having an amino acid sequence of SEQ ID NO:445, a VL CDR2 having an amino acid sequence of SEQ ID NO:446, and a VL CDR3 having an amino acid sequence of SEQ ID NO:447. In some embodiments, the first binding domain comprises a VH having an amino acid sequence of SEQ ID NO:104. In some embodiments, the first binding domain comprises a VL having an amino acid sequence of SEQ ID NO:105. In some embodiments, the first binding domain comprises a VH having an amino acid sequence of SEQ ID NO:104, and a VL having an amino acid sequence of SEQ ID NO:105. In some embodiments, the first binding domain comprises an amino acid sequence of SEQ ID NO:106. In some embodiments, the first binding domain comprises a VH comprising an amino acid sequence having at least 95% identity to the amino acid sequence of SEQ ID NO:104. In some embodiments, the first binding domain comprises a VL comprising an amino acid sequence having at least 95% identity to the amino acid sequence of SEQ ID NO:105. In some embodiments, the first binding domain comprises a VH comprising an amino acid sequence having at least 95% identity to the amino acid sequence of SEQ ID NO:104, and a VL comprising an amino acid sequence having at least 95% identity to the amino acid sequence of SEQ ID NO:105. In some embodiments, the first binding domain comprises an amino acid sequence having at least 95% identity to an amino acid sequence of SEQ ID NO:106.

In one embodiment of a multispecific TRGV9 antibody provided herein, the first binding domain that binds to TRGV9 comprises a VH and VL amino acid sequence of VG9SB10SC1087_P18_D08. In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:113. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:114. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:113; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:114. In one embodiment, the first binding domain comprises: (i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109, and (ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO:112. In one embodiment, the first binding domain comprises: (i) a VH comprising a VH CDR1 having an amino acid sequence of SEQ ID NO:448, a VH CDR2 having an amino acid sequence of SEQ ID NO:449, and a VH CDR3 having an amino acid sequence of SEQ ID NO:450; and (ii) a VL comprising a VL CDR1 having an amino acid sequence of SEQ ID NO:451, a VL CDR2 having an amino acid sequence of SEQ ID NO:452, and a VL CDR3 having an amino acid sequence of SEQ ID NO:453. In one embodiment, the first binding domain comprises: (i) a VH comprising a VH CDR1 having an amino acid sequence of SEQ ID NO:454, a VH CDR2 having an amino acid sequence of SEQ ID NO:455, and a VH CDR3 having an amino acid sequence of SEQ ID NO:456; and (ii) a VL comprising a VL CDR1 having an amino acid sequence of SEQ ID NO:457, a VL CDR2 having an amino acid sequence of SEQ ID NO:458, and a VL CDR3 having an amino acid sequence of SEQ ID NO:459. In one embodiment, the first binding domain comprises: (i) a VH comprising a VH CDR1 having an amino acid sequence of SEQ ID NO:460, a VH CDR2 having an amino acid sequence of SEQ ID NO:461, and a VH CDR3 having an amino acid sequence of SEQ ID NO:462; and (ii) a VL comprising a VL CDR1 having an amino acid sequence of SEQ ID NO:463, a VL CDR2 having an amino acid sequence of SEQ ID NO:464, and a VL CDR3 having an amino acid sequence of SEQ ID NO:465. In one embodiment, the first binding domain comprises: (i) a VH comprising a VH CDR1 having an amino acid sequence of SEQ ID NO:466, a VH CDR2 having an amino acid sequence of SEQ ID NO:467, and a VH CDR3 having an amino acid sequence of SEQ ID NO:468; and (ii) a VL comprising a VL CDR1 having an amino acid sequence of SEQ ID NO:469, a VL CDR2 having an amino acid sequence of SEQ ID NO:470, and a VL CDR3 having an amino acid sequence of SEQ ID NO:471. In one embodiment, the first binding domain comprises: (i) a VH comprising a VH CDR1 having an amino acid sequence of SEQ ID NO:466, a VH CDR2 having an amino acid sequence of SEQ ID NO:716, and a VH CDR3 having an amino acid sequence of SEQ ID NO:717; and (ii) a VL comprising a VL CDR1 having an amino acid sequence of SEQ ID NO:469, a VL CDR2 having an amino acid sequence of SEQ ID NO:470, and a VL CDR3 having an amino acid sequence of SEQ ID NO:471. In one embodiment, the first binding domain comprises: (i) a VH comprising a VH CDR1 having an amino acid sequence of SEQ ID NO:472, a VH CDR2 having an amino acid sequence of SEQ ID NO:473, and a VH CDR3 having an amino acid sequence of SEQ ID NO:474; and (ii) a VL comprising a VL CDR1 having an amino acid sequence of SEQ ID NO:475, a VL CDR2 having an amino acid sequence of SEQ ID NO:476, and a VL CDR3 having an amino acid sequence of SEQ ID NO:477. In one embodiment, the first binding domain comprises: (i) a VH comprising a VH CDR1 having an amino acid sequence of SEQ ID NO:478, a VH CDR2 having an amino acid sequence of SEQ ID NO:479, and a VH CDR3 having an amino acid sequence of SEQ ID NO:480; and (ii) a VL comprising a VL CDR1 having an amino acid sequence of SEQ ID NO:481, a VL CDR2 having an amino acid sequence of SEQ ID NO:482, and a VL CDR3 having an amino acid sequence of SEQ ID NO:483. In some embodiments, the first binding domain comprises a VH having an amino acid sequence of SEQ ID NO:113. In some embodiments, the first binding domain comprises a VL having an amino acid sequence of SEQ ID NO:114. In some embodiments, the first binding domain comprises a VH having an amino acid sequence of SEQ ID NO:113, and a VL having an amino acid sequence of SEQ ID NO:114. In some embodiments, the first binding domain comprises a heavy chain having an amino acid sequence of SEQ ID NO:115. In some embodiments, the first binding domain comprises a light chain having an amino acid sequence of SEQ ID NO:116. In some embodiments, the first binding domain comprises a heavy chain having an amino acid sequence of SEQ ID NO:115, and a light chain having an amino acid sequence of SEQ ID NO:116. In some embodiments, the first binding domain comprises a VH comprising an amino acid sequence having at least 95% identity to the amino acid sequence of SEQ ID NO:113. In some embodiments, the first binding domain comprises a VL comprising an amino acid sequence having at least 95% identity to the amino acid sequence of SEQ ID NO:114. In some embodiments, the first binding domain comprises a VH comprising an amino acid sequence having at least 95% identity to the amino acid sequence of SEQ ID NO:113, and a VL comprising an amino acid sequence having at least 95% identity to the amino acid sequence of SEQ ID NO:114. In some embodiments, the first binding domain comprises a heavy chain comprising an amino acid sequence having at least 95% identity to the amino acid sequence of SEQ ID NO:115. In some embodiments, the first binding domain comprises a light chain comprising an amino acid sequence having at least 95% identity to the amino acid sequence of SEQ ID NO:116. In some embodiments, the first binding domain comprises a heavy chain comprising an amino acid sequence having at least 95% identity to the amino acid sequence of SEQ ID NO:115, and a light chain comprising an amino acid sequence having at least 95% identity to the amino acid sequence of SEQ ID NO:116.

In one embodiment of a multispecific TRGV9 antibody provided herein, the first binding domain that binds to TRGV9 comprises a VH and VL amino acid sequence of VG9SB10SC1087_P18_C12. In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:123. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:124. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:123; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:124. In one embodiment, the first binding domain comprises: (i) a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119, and (ii) a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO:122. In one embodiment, the first binding domain comprises: (i) a VH comprising a VH CDR1 having an amino acid sequence of SEQ ID NO:484, a VH CDR2 having an amino acid sequence of SEQ ID NO:485, and a VH CDR3 having an amino acid sequence of SEQ ID NO:486; and (ii) a VL comprising a VL CDR1 having an amino acid sequence of SEQ ID NO:487, a VL CDR2 having an amino acid sequence of SEQ ID NO:488, and a VL CDR3 having an amino acid sequence of SEQ ID NO:489. In one embodiment, the first binding domain comprises: (i) a VH comprising a VH CDR1 having an amino acid sequence of SEQ ID NO:490, a VH CDR2 having an amino acid sequence of SEQ ID NO:491, and a VH CDR3 having an amino acid sequence of SEQ ID NO:492; and (ii) a VL comprising a VL CDR1 having an amino acid sequence of SEQ ID NO:493, a VL CDR2 having an amino acid sequence of SEQ ID NO:494, and a VL CDR3 having an amino acid sequence of SEQ ID NO:495. In one embodiment, the first binding domain comprises: (i) a VH comprising a VH CDR1 having an amino acid sequence of SEQ ID NO:496, a VH CDR2 having an amino acid sequence of SEQ ID NO:497, and a VH CDR3 having an amino acid sequence of SEQ ID NO:498; and (ii) a VL comprising a VL CDR1 having an amino acid sequence of SEQ ID NO:499, a VL CDR2 having an amino acid sequence of SEQ ID NO:500, and a VL CDR3 having an amino acid sequence of SEQ ID NO:501. In one embodiment, the first binding domain comprises: (i) a VH comprising a VH CDR1 having an amino acid sequence of SEQ ID NO:502, a VH CDR2 having an amino acid sequence of SEQ ID NO:503, and a VH CDR3 having an amino acid sequence of SEQ ID NO:504; and (ii) a VL comprising a VL CDR1 having an amino acid sequence of SEQ ID NO:505, a VL CDR2 having an amino acid sequence of SEQ ID NO:506, and a VL CDR3 having an amino acid sequence of SEQ ID NO:507. In one embodiment, the first binding domain comprises: (i) a VH comprising a VH CDR1 having an amino acid sequence of SEQ ID NO:502, a VH CDR2 having an amino acid sequence of SEQ ID NO:718, and a VH CDR3 having an amino acid sequence of SEQ ID NO:719; and (ii) a VL comprising a VL CDR1 having an amino acid sequence of SEQ ID NO:505, a VL CDR2 having an amino acid sequence of SEQ ID NO:506, and a VL CDR3 having an amino acid sequence of SEQ ID NO:507. In one embodiment, the first binding domain comprises: (i) a VH comprising a VH CDR1 having an amino acid sequence of SEQ ID NO:508, a VH CDR2 having an amino acid sequence of SEQ ID NO:509, and a VH CDR3 having an amino acid sequence of SEQ ID NO:510; and (ii) a VL comprising a VL CDR1 having an amino acid sequence of SEQ ID NO:511, a VL CDR2 having an amino acid sequence of SEQ ID NO:512, and a VL CDR3 having an amino acid sequence of SEQ ID NO:513. In one embodiment, the first binding domain comprises: (i) a VH comprising a VH CDR1 having an amino acid sequence of SEQ ID NO:514, a VH CDR2 having an amino acid sequence of SEQ ID NO:515, and a VH CDR3 having an amino acid sequence of SEQ ID NO:516; and (ii) a VL comprising a VL CDR1 having an amino acid sequence of SEQ ID NO:517, a VL CDR2 having an amino acid sequence of SEQ ID NO:518, and a VL CDR3 having an amino acid sequence of SEQ ID NO:519. In some embodiments, the first binding domain comprises a VH having an amino acid sequence of SEQ ID NO:123. In some embodiments, the first binding domain comprises a VL having an amino acid sequence of SEQ ID NO:124. In some embodiments, the first binding domain comprises a VH having an amino acid sequence of SEQ ID NO:123, and a VL having an amino acid sequence of SEQ ID NO:124. In some embodiments, the first binding domain comprises a heavy chain having an amino acid sequence of SEQ ID NO:125. In some embodiments, the first binding domain comprises a light chain having an amino acid sequence of SEQ ID NO:126. In some embodiments, the first binding domain comprises a heavy chain having an amino acid sequence of SEQ ID NO:125, and a light chain having an amino acid sequence of SEQ ID NO:126. In some embodiments, the first binding domain comprises a VH comprising an amino acid sequence having at least 95% identity to the amino acid sequence of SEQ ID NO:123. In some embodiments, the first binding domain comprises a VL comprising an amino acid sequence having at least 95% identity to the amino acid sequence of SEQ ID NO:124. In some embodiments, the first binding domain comprises a VH comprising an amino acid sequence having at least 95% identity to the amino acid sequence of SEQ ID NO:123, and a VL comprising an amino acid sequence having at least 95% identity to the amino acid sequence of SEQ ID NO:124. In some embodiments, the first binding domain comprises a heavy chain comprising an amino acid sequence having at least 95% identity to the amino acid sequence of SEQ ID NO:125. In some embodiments, the first binding domain comprises a light chain comprising an amino acid sequence having at least 95% identity to the amino acid sequence of SEQ ID NO:126. In some embodiments, the first binding domain comprises a heavy chain comprising an amino acid sequence having at least 95% identity to the amino acid sequence of SEQ ID NO:125, and a light chain comprising an amino acid sequence having at least 95% identity to the amino acid sequence of SEQ ID NO:126.

In one embodiment of a multispecific TRGV9 antibody provided herein, the first binding domain that binds to TRGV9 comprises a VH and VL amino acid sequence of VG9SB10SC1087_P19_C03. In one embodiment, the first binding domain comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133. In one embodiment, the first binding domain comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the first binding domain comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the first binding domain comprises: (i) a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO:129, and (ii) a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO:132. In one embodiment, the first binding domain comprises: (i) a VH comprising a VH CDR1 having an amino acid sequence of SEQ ID NO:520, a VH CDR2 having an amino acid sequence of SEQ ID NO:521, and a VH CDR3 having an amino acid sequence of SEQ ID NO:522; and (ii) a VL comprising a VL CDR1 having an amino acid sequence of SEQ ID NO:523, a VL CDR2 having an amino acid sequence of SEQ ID NO:524, and a VL CDR3 having an amino acid sequence of SEQ ID NO:525. In one embodiment, the first binding domain comprises: (i) a VH comprising a VH CDR1 having an amino acid sequence of SEQ ID NO:526, a VH CDR2 having an amino acid sequence of SEQ ID NO:527, and a VH CDR3 having an amino acid sequence of SEQ ID NO:528; and (ii) a VL comprising a VL CDR1 having an amino acid sequence of SEQ ID NO:529, a VL CDR2 having an amino acid sequence of SEQ ID NO:530, and a VL CDR3 having an amino acid sequence of SEQ ID NO:531. In one embodiment, the first binding domain comprises: (i) a VH comprising a VH CDR1 having an amino acid sequence of SEQ ID NO:532, a VH CDR2 having an amino acid sequence of SEQ ID NO:533, and a VH CDR3 having an amino acid sequence of SEQ ID NO:534; and (ii) a VL comprising a VL CDR1 having an amino acid sequence of SEQ ID NO:535, a VL CDR2 having an amino acid sequence of SEQ ID NO:536, and a VL CDR3 having an amino acid sequence of SEQ ID NO:537. In one embodiment, the first binding domain comprises: (i) a VH comprising a VH CDR1 having an amino acid sequence of SEQ ID NO:538, a VH CDR2 having an amino acid sequence of SEQ ID NO:539, and a VH CDR3 having an amino acid sequence of SEQ ID NO:540; and (ii) a VL comprising a VL CDR1 having an amino acid sequence of SEQ ID NO:541, a VL CDR2 having an amino acid sequence of SEQ ID NO:542, and a VL CDR3 having an amino acid sequence of SEQ ID NO:543. In one embodiment, the first binding domain comprises: (i) a VH comprising a VH CDR1 having an amino acid sequence of SEQ ID NO:538, a VH CDR2 having an amino acid sequence of SEQ ID NO:720, and a VH CDR3 having an amino acid sequence of SEQ ID NO:721; and (ii) a VL comprising a VL CDR1 having an amino acid sequence of SEQ ID NO:541, a VL CDR2 having an amino acid sequence of SEQ ID NO:542, and a VL CDR3 having an amino acid sequence of SEQ ID NO:543. In one embodiment, the first binding domain comprises: (i) a VH comprising a VH CDR1 having an amino acid sequence of SEQ ID NO:544, a VH CDR2 having an amino acid sequence of SEQ ID NO:545, and a VH CDR3 having an amino acid sequence of SEQ ID NO:546; and (ii) a VL comprising a VL CDR1 having an amino acid sequence of SEQ ID NO:547, a VL CDR2 having an amino acid sequence of SEQ ID NO:548, and a VL CDR3 having an amino acid sequence of SEQ ID NO:549. In one embodiment, the first binding domain comprises: (i) a VH comprising a VH CDR1 having an amino acid sequence of SEQ ID NO:550, a VH CDR2 having an amino acid sequence of SEQ ID NO:551, and a VH CDR3 having an amino acid sequence of SEQ ID NO:552; and (ii) a VL comprising a VL CDR1 having an amino acid sequence of SEQ ID NO:553, a VL CDR2 having an amino acid sequence of SEQ ID NO:554, and a VL CDR3 having an amino acid sequence of SEQ ID NO:555. In some embodiments, the first binding domain comprises a VH having an amino acid sequence of SEQ ID NO:133. In some embodiments, the first binding domain comprises a VL having an amino acid sequence of SEQ ID NO:134. In some embodiments, the first binding domain comprises a VH having an amino acid sequence of SEQ ID NO:133, and a VL having an amino acid sequence of SEQ ID NO:134. In some embodiments, the first binding domain comprises a heavy chain having an amino acid sequence of SEQ ID NO:135. In some embodiments, the first binding domain comprises a light chain having an amino acid sequence of SEQ ID NO:136. In some embodiments, the first binding domain comprises a heavy chain having an amino acid sequence of SEQ ID NO:135, and a light chain having an amino acid sequence of SEQ ID NO:136. In some embodiments, the first binding domain comprises a VH comprising an amino acid sequence having at least 95% identity to the amino acid sequence of SEQ ID NO:133. In some embodiments, the first binding domain comprises a VL comprising an amino acid sequence having at least 95% identity to the amino acid sequence of SEQ ID NO:134. In some embodiments, the first binding domain comprises a VH comprising an amino acid sequence having at least 95% identity to the amino acid sequence of SEQ ID NO:133, and a VL comprising an amino acid sequence having at least 95% identity to the amino acid sequence of SEQ ID NO:134. In some embodiments, the first binding domain comprises a heavy chain comprising an amino acid sequence having at least 95% identity to the amino acid sequence of SEQ ID NO:135. In some embodiments, the first binding domain comprises a light chain comprising an amino acid sequence having at least 95% identity to the amino acid sequence of SEQ ID NO:136. In some embodiments, the first binding domain comprises a heavy chain comprising an amino acid sequence having at least 95% identity to the amino acid sequence of SEQ ID NO:135, and a light chain comprising an amino acid sequence having at least 95% identity to the amino acid sequence of SEQ ID NO:136.

In some embodiments, the VH CDR1, VH CDR2 and VH CDR3 amino acid sequences of the first binding domain that binds TRGV9 are according to the Kabat numbering system. In some embodiments, the VH CDR1, VH CDR2 and VH CDR3 amino acid sequences of the first binding domain that binds TRGV9 are according to the Chothia numbering system. In some embodiments, the VH CDR1, VH CDR2 and VH CDR3 amino acid sequences of the first binding domain that binds TRGV9 are according to the AbM numbering system. In some embodiments, the VH CDR1, VH CDR2 and VH CDR3 amino acid sequences of the first binding domain that binds TRGV9 are according to the Contact numbering system. In some embodiments, the VH CDR1, VH CDR2 and VH CDR3 amino acid sequences of the first binding domain that binds TRGV9 are according to the IMGT numbering system. In some embodiments, the VH CDR1, VH CDR2 and VH CDR3 amino acid sequences of the first binding domain that binds TRGV9 are according to the Exemplary numbering system. In some embodiments, the VL CDR1, VL CDR2 and VL CDR3 amino acid sequences of the first binding domain that binds TRGV9 are according to the Kabat numbering system. In some embodiments, the VL CDR1, VL CDR2 and VL CDR3 amino acid sequences of the first binding domain that binds TRGV9 are according to the Chothia numbering system. In some embodiments, the VL CDR1, VL CDR2 and VL CDR3 amino acid sequences of the first binding domain that binds TRGV9 are according to the AbM numbering system. In some embodiments, the VL CDR1, VL CDR2 and VL CDR3 amino acid sequences of the first binding domain that binds TRGV9 are according to the Contact numbering system. In some embodiments, the VL CDR1, VL CDR2 and VL CDR3 amino acid sequences of the first binding domain that binds TRGV9 are according to the IMGT numbering system. In some embodiments, the VL CDR1, VL CDR2 and VL CDR3 amino acid sequences of the first binding domain that binds TRGV9 are according to the Exemplary numbering system. In some embodiments, the VH CDR1, VH CDR2, VH CDR3, VL CDR1, VL CDR2, and VL CDR3 amino acid sequences of the first binding domain that binds TRGV9 are according to the Kabat numbering system. In some embodiments, the VH CDR1, VH CDR2, VH CDR3, VL CDR1, VL CDR2, and VL CDR3 amino acid sequences of the first binding domain that binds TRGV9 are according to the Chothia numbering system. In some embodiments, the VH CDR1, VH CDR2, VH CDR3, VL CDR1, VL CDR2, and VL CDR3 amino acid sequences of the first binding domain that binds TRGV9 are according to the AbM numbering system. In some embodiments, the VH CDR1, VH CDR2, VH CDR3, VL CDR1, VL CDR2, and VL CDR3 amino acid sequences of the first binding domain that binds TRGV9 are according to the Contact numbering system. In some embodiments, the VH CDR1, VH CDR2, VH CDR3, VL CDR1, VL CDR2, and VL CDR3 amino acid sequences of the first binding domain that binds TRGV9 are according to the IMGT numbering system. In some embodiments, the VH CDR1, VH CDR2, VH CDR3, VL CDR1, VL CDR2, and VL CDR3 amino acid sequences of the first binding domain that binds TRGV9 are according to the Exemplary numbering system.

In some embodiments, the first binding domain binds a TRGV9 antigen. In some embodiments, the first binding domain binds a TRGV9 epitope. In some embodiments, the first binding domain specifically binds to TRGV9. In some embodiments, the VH CDR1, VH CDR2, VH CDR3, VL CDR1, VL CDR2 and VL CDR3 of the first binding domain form a binding site for an antigen of the TRGV9. In some embodiments, the VH CDR1, VH CDR2, VH CDR3, VL CDR1, VL CDR2 and VL CDR3 of the first binding domain form a binding site for an epitope of the TRGV9. In some embodiments, the VH region and the VL region of the first binding domain form a binding site for an antigen of TRGV9. In some embodiments, the VH region and the VL region of the first binding domain form a binding site for an epitope of the TRGV9. In some embodiments, the heavy chain and the light chain of the first binding domain form a binding site for an antigen of TRGV9. In some embodiments, the heavy chain and the light chain of the second binding domain form a binding site for an epitope of TRGV9.

In some embodiments, the TRGV9 is present on the surface of a cell. In specific embodiment, the TRGV9 is present on the surface of a T cell. In a specific embodiment, the T cell is a γδ T cell.

In some embodiments of the multispecific TRGV9 antibodies provided herein, the second target is CD123. In one embodiment of the multispecific TRGV9 antibodies provided herein, the second binding domain that binds CD123 comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:15. In one embodiment of the multispecific TRGV9 antibodies provided herein, the second binding domain that binds CD123 comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:16. In one embodiment of the multispecific TRGV9 antibodies provided herein, the second binding domain that binds CD123 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:15; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:16. In one embodiment, the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In one embodiment, the second binding domain comprises: (i) a VH comprising a VH CDR1 having an amino acid sequence of SEQ ID NO:556, a VH CDR2 having an amino acid sequence of SEQ ID NO:557, and a VH CDR3 having an amino acid sequence of SEQ ID NO:558; and (ii) a VL comprising a VL CDR1 having an amino acid sequence of SEQ ID NO:559, a VL CDR2 having an amino acid sequence of SEQ ID NO:560, and a VL CDR3 having an amino acid sequence of SEQ ID NO:561. In one embodiment, the second binding domain comprises: (i) a VH comprising a VH CDR1 having an amino acid sequence of SEQ ID NO:562, a VH CDR2 having an amino acid sequence of SEQ ID NO:563, and a VH CDR3 having an amino acid sequence of SEQ ID NO:564; and (ii) a VL comprising a VL CDR1 having an amino acid sequence of SEQ ID NO:565, a VL CDR2 having an amino acid sequence of SEQ ID NO:566, and a VL CDR3 having an amino acid sequence of SEQ ID NO:567. In one embodiment, the second binding domain comprises: (i) a VH comprising a VH CDR1 having an amino acid sequence of SEQ ID NO:568, a VH CDR2 having an amino acid sequence of SEQ ID NO:569, and a VH CDR3 having an amino acid sequence of SEQ ID NO:570; and (ii) a VL comprising a VL CDR1 having an amino acid sequence of SEQ ID NO:571, a VL CDR2 having an amino acid sequence of SEQ ID NO:572, and a VL CDR3 having an amino acid sequence of SEQ ID NO:573. In one embodiment, the second binding domain comprises: (i) a VH comprising a VH CDR1 having an amino acid sequence of SEQ ID NO:574, a VH CDR2 having an amino acid sequence of SEQ ID NO:575, and a VH CDR3 having an amino acid sequence of SEQ ID NO:576; and (ii) a VL comprising a VL CDR1 having an amino acid sequence of SEQ ID NO:577, a VL CDR2 having an amino acid sequence of SEQ ID NO:578, and a VL CDR3 having an amino acid sequence of SEQ ID NO:579. In one embodiment, the second binding domain comprises: (i) a VH comprising a VH CDR1 having an amino acid sequence of SEQ ID NO:574, a VH CDR2 having an amino acid sequence of SEQ ID NO:722, and a VH CDR3 having an amino acid sequence of SEQ ID NO:723; and (ii) a VL comprising a VL CDR1 having an amino acid sequence of SEQ ID NO:577, a VL CDR2 having an amino acid sequence of SEQ ID NO:578, and a VL CDR3 having an amino acid sequence of SEQ ID NO:579. In one embodiment, the second binding domain comprises: (i) a VH comprising a VH CDR1 having an amino acid sequence of SEQ ID NO:580, a VH CDR2 having an amino acid sequence of SEQ ID NO:581, and a VH CDR3 having an amino acid sequence of SEQ ID NO:582; and (ii) a VL comprising a VL CDR1 having an amino acid sequence of SEQ ID NO:583, a VL CDR2 having an amino acid sequence of SEQ ID NO:584, and a VL CDR3 having an amino acid sequence of SEQ ID NO:585. In one embodiment, the second binding domain comprises: (i) a VH comprising a VH CDR1 having an amino acid sequence of SEQ ID NO:586, a VH CDR2 having an amino acid sequence of SEQ ID NO:587, and a VH CDR3 having an amino acid sequence of SEQ ID NO:588; and (ii) a VL comprising a VL CDR1 having an amino acid sequence of SEQ ID NO:589, a VL CDR2 having an amino acid sequence of SEQ ID NO:590, and a VL CDR3 having an amino acid sequence of SEQ ID NO:591. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence having at least 95% identity to an amino acid sequence of SEQ ID NO:18.

The TRGV9×CD123 multispecific antibody can comprise a first binding domain comprising any TRGV9 antibody provided herein. The TRGV9×CD123 multispecific antibody can further comprise a second binding domain comprising any CD123 antibody, including any CD123 antibody provided herein.

In some embodiments of the multispecific TRGV9 antibodies provided herein, the second target is CD33. In one embodiment, the second target is the C2 domain of CD33. the second target is the V domain of CD33. In one embodiment of the multispecific TRGV9 antibodies provided herein, the second binding domain that binds to CD33 comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:43. In one embodiment of the multispecific TRGV9 antibodies provided herein, the second binding domain that binds to CD33 comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:44. In one embodiment of the multispecific TRGV9 antibodies provided herein, the second binding domain that binds to CD33 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:43; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:44. In some embodiments, the second binding domain that binds to CD33 has a VH CDR1, VH CDR2, VH CDR3, VL CDR1, VL CDR2, and VL CDR3, having the amino acid sequence of SEQ ID NOs:37, 38, 39, 40, 41, and 42, respectively. In some embodiments, the second binding domain that binds to CD33 has a VH CDR1, VH CDR2, VH CDR3, VL CDR1, VL CDR2, and VL CDR3, having the amino acid sequence of SEQ ID NOs:592, 593, 594, 595, 596, and 597, respectively. In some embodiments, the second binding domain that binds to CD33 has a VH CDR1, VH CDR2, VH CDR3, VL CDR1, VL CDR2, and VL CDR3, having the amino acid sequence of SEQ ID NOs:598, 599, 600, 601, 602, and 603, respectively. In some embodiments, the second binding domain that binds to CD33 has a VH CDR1, VH CDR2, VH CDR3, VL CDR1, VL CDR2, and VL CDR3, having the amino acid sequence of SEQ ID NOs:604, 605, 606, 607, 608, and 609, respectively. In some embodiments, the second binding domain that binds to CD33 has a VH CDR1, VH CDR2, VH CDR3, VL CDR1, VL CDR2, and VL CDR3, having the amino acid sequence of SEQ ID NOs:610, 611, 612, 613, 614, and 615, respectively. In some embodiments, the second binding domain that binds to CD33 has a VH CDR1, VH CDR2, VH CDR3, VL CDR1, VL CDR2, and VL CDR3, having the amino acid sequence of SEQ ID NOs:616, 617, 618, 619, 620, and 621, respectively. In some embodiments, the second binding domain that binds to CD33 has a VH CDR1, VH CDR2, VH CDR3, VL CDR1, VL CDR2, and VL CDR3, having the amino acid sequence of SEQ ID NOs:622, 623, 624, 625, 626, and 627, respectively. In certain embodiments, the second binding domain that binds CD33 has a VH having the amino acid sequence of SEQ ID NO:43. In certain embodiments, the second binding domain that binds CD33 has a VL having the amino acid sequence of SEQ ID NO:44. In some embodiments, the second binding domain that binds CD33 has a VH having the amino acid sequence of SEQ ID NO:43, and a VL having the amino acid sequence of SEQ ID NO:44. In certain embodiments, the second binding domain that binds CD33 has a heavy chain having the amino acid sequence of SEQ ID NO:47. In certain embodiments, the second binding domain that binds CD33 has a light chain having the amino acid sequence of SEQ ID NO:48. In some embodiments, the second binding domain that binds CD33 has a heavy chain having the amino acid sequence of SEQ ID NO:47, and a light chain having the amino acid sequence of SEQ ID NO:48. In certain embodiments, the second binding domain that binds CD33 has an amino acid sequence of SEQ ID NO:45. In certain embodiments, the second binding domain that binds CD33 has a VH having at least 95% sequence identity to an amino acid sequence of SEQ ID NO:43. In certain embodiments, the second binding domain that binds CD33 has a VL having at least 95% sequence identity to an amino acid sequence of SEQ ID NO:44. In some embodiments, the second binding domain that binds CD33 has a VH having at least 95% sequence identity to an amino acid sequence of SEQ ID NO:43, and a VL having at least 95% sequence identity to an amino acid sequence of SEQ ID NO:44. In certain embodiments, the second binding domain that binds CD33 has a heavy chain having at least 95% sequence identity to an amino acid sequence of SEQ ID NO:47. In certain embodiments, the second binding domain that binds CD33 has a light chain having at least 95% sequence identity to an amino acid sequence of SEQ ID NO:48. In some embodiments, the second binding domain that binds CD33 has a heavy chain having at least 95% sequence identity to an amino acid sequence of SEQ ID NO:47, and a light chain having at least 95% sequence identity to an amino acid sequence of SEQ ID NO:48. In certain embodiments, the second binding domain that binds CD33 has an amino acid sequence having at least 95% sequence identity to an amino acid sequence of SEQ ID NO:45.

Additional CD33 antibodies that can be used for the TRGV9 multispecific antibodies provided herein include AMG330 and AMG673 (Amgen; Friedrich et al., 2014), AMV564 (Amphivena; U.S. Pat. No. 9,803,029), IMGN779 (Immunogen; U.S. Pat. No. 9,359,442), BI836858 (Boehringer Ingelheim; Vasu et al., 2016), Actimab (Actinium Pharma), gemtuzumab (Godwin, Gale, & Walter, 2017), and SGN33A (Seattle Genetics). In some embodiments of the multispecific TRGV9 antibodies provided herein, the second binding domain that binds CD33 comprises the VH CDR1-3 and VL CDR1-3 of AMG330. In some embodiments of the multispecific TRGV9 antibodies provided herein, the second binding domain that binds CD33 comprises the VH CDR1-3 and VL CDR1-3 of AMG673. In some embodiments of the multispecific TRGV9 antibodies provided herein, the second binding domain that binds CD33 comprises the VH CDR1-3 and VL CDR1-3 of AMV564. In some embodiments of the multispecific TRGV9 antibodies provided herein, the second binding domain that binds CD33 comprises the VH CDR1-3 and VL CDR1-3 of IMGN779. In some embodiments of the multispecific TRGV9 antibodies provided herein, the second binding domain that binds CD33 comprises the VH CDR1-3 and VL CDR1-3 of BI836858. In some embodiments of the multispecific TRGV9 antibodies provided herein, the second binding domain that binds CD33 comprises the VH CDR1-3 and VL CDR1-3 of Actimab. In some embodiments of the multispecific TRGV9 antibodies provided herein, the second binding domain that binds CD33 comprises the VH CDR1-3 and VL CDR1-3 of gentuzimab. In some embodiments of the multispecific TRGV9 antibodies provided herein, the second binding domain that binds CD33 comprises the VH CDR1-3 and VL CDR1-3 of SGN33A.

The TRGV9×CD33 multispecific antibody can comprise a first binding domain comprising any TRGV9 antibody provided herein. The TRGV9×CD33 multispecific antibody can further comprise a second binding domain comprising any CD33 antibody, including any CD33 antibody provided herein.

In some embodiments of the multispecific TRGV9 antibodies provided herein, the second target is TRBC1. In one embodiment of the multispecific TRGV9 antibodies provided herein, the second binding domain that binds to TRBC1 comprises a VH CDR1, a VH CDR2, and a VH CDR3 having an amino acid sequence of a VH CDR1, a VH CDR2, and a VH CDR3, respectively, of a VH having an amino acid sequence of SEQ ID NO:55. In one embodiment of the multispecific TRGV9 antibodies provided herein, the second binding domain that binds to TRBC1 comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:56. In one embodiment of the multispecific TRGV9 antibodies provided herein, the second binding domain that binds to TRBC1 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:55; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:56. In some embodiments, the second binding domain that binds to TRBC1 has a VH CDR1, VH CDR2, VH CDR3, VL CDR1, VL CDR2, and VL CDR3, having the amino acid sequence of SEQ ID NOs:49, 50, 51, 52, 53 and 54, respectively. In some embodiments, the second binding domain that binds to TRBC1 has a VH CDR1, VH CDR2, VH CDR3, VL CDR1, VL CDR2, and VL CDR3, having the amino acid sequence of SEQ ID NOs:628, 629, 630, 631, 632, and 633, respectively. In some embodiments, the second binding domain that binds to TRBC1 has a VH CDR1, VH CDR2, VH CDR3, VL CDR1, VL CDR2, and VL CDR3, having the amino acid sequence of SEQ ID NOs:634, 635, 636, 637, 638, and 639, respectively. In some embodiments, the second binding domain that binds to TRBC1 has a VH CDR1, VH CDR2, VH CDR3, VL CDR1, VL CDR2, and VL CDR3, having the amino acid sequence of SEQ ID NOs:640, 641, 642, 643, 644, 645, respectively. In some embodiments, the second binding domain that binds to TRBC1 has a VH CDR1, VH CDR2, VH CDR3, VL CDR1, VL CDR2, and VL CDR3, having the amino acid sequence of SEQ ID NOs:646, 647, 648, 649, 650, and 651, respectively. In some embodiments, the second binding domain that binds to TRBC1 has a VH CDR1, VH CDR2, VH CDR3, VL CDR1, VL CDR2, and VL CDR3, having the amino acid sequence of SEQ ID NOs:652, 653, 654, 655, 656, and 657, respectively. In some embodiments, the second binding domain that binds to TRBC1 has a VH CDR1, VH CDR2, VH CDR3, VL CDR1, VL CDR2, and VL CDR3, having the amino acid sequence of SEQ ID NOs:658, 659, 660, 661, 662, and 633, respectively. In certain embodiments, the second binding domain that binds TRBC1 has a VH having the amino acid sequence of SEQ ID NO:55. In certain embodiments, the second binding domain that binds TRBC1 has a VL having the amino acid sequence of SEQ ID NO:56. In some embodiments, the second binding domain that binds TRBC1 has a VH having the amino acid sequence of SEQ ID NO:55, and a VL having the amino acid sequence of SEQ ID NO:56. In certain embodiments, the second binding domain that binds TRBC1 has a heavy chain having the amino acid sequence of SEQ ID NO:58. In certain embodiments, the second binding domain that binds TRBC1 has a light chain having the amino acid sequence of SEQ ID NO:59. In some embodiments, the second binding domain that binds TRBC1 has a heavy chain having the amino acid sequence of SEQ ID NO:58, and a light chain having the amino acid sequence of SEQ ID NO:59. In certain embodiments, the second binding domain that binds TRBC1 has an amino acid sequence of SEQ ID NO:57. In certain embodiments, the second binding domain that binds TRBC1 has a VH having at least 95% sequence identity to an amino acid sequence of SEQ ID NO:55. In certain embodiments, the second binding domain that binds TRBC1 has a VL having at least 95% sequence identity to an amino acid sequence of SEQ ID NO:56. In some embodiments, the second binding domain that binds TRBC1 has a VH having at least 95% sequence identity to an amino acid sequence of SEQ ID NO:55, and a VL having at least 95% sequence identity to an amino acid sequence of SEQ ID NO:56. In certain embodiments, the second binding domain that binds TRBC1 has a heavy chain having at least 95% sequence identity to an amino acid sequence of SEQ ID NO:58. In certain embodiments, the second binding domain that binds TRBC1 has a light chain having at least 95% sequence identity to an amino acid sequence of SEQ ID NO:59. In some embodiments, the second binding domain that binds TRBC1 has a heavy chain having at least 95% sequence identity to an amino acid sequence of SEQ ID NO:58, and a light chain having at least 95% sequence identity to an amino acid sequence of SEQ ID NO:59. In certain embodiments, the second binding domain that binds TRBC1 has an amino acid sequence having at least 95% sequence identity to an amino acid sequence of SEQ ID NO:57.

The TRGV9×TRBC1 multispecific antibody can comprise a first binding domain comprising any TRGV9 antibody provided herein. The TRGV9×TRBC1 multispecific antibody can further comprise a second binding domain comprising any TRBC1 antibody, including any TRBC1 antibody provided herein.

In some embodiments of the multispecific TRGV9 antibodies provided herein, the second target is BCMA. In one embodiment, the second binding domain binds to BCMA. In some embodiments, the second binding domain that binds BCMA comprises a VH comprising a VH CDR1, a VH CDR2, and a VH CDR3 having amino acid sequences of the VH CDR1, VH CDR2, and VH CDR3, respectively, of SEQ ID NO:143. In some embodiments, the second binding domain that binds BCMA comprises a VL comprising a VL CDR1, a VL CDR2, and a VL CDR3 having amino acid sequences of the VL CDR1, VL CDR2, and VL CDR3, respectively, of SEQ ID NO:144. In some embodiments, the second binding domain that binds BCMA comprises: (i) a VH comprising a VH CDR1, a VH CDR2, and a VH CDR3 having amino acid sequences of the VH CDR1, VH CDR2, and VH CDR3, respectively, of SEQ ID NO:143; and (ii) a VL comprising a VL CDR1, a VL CDR2, and a VL CDR3 having amino acid sequences of the VL CDR1, VL CDR2, and VL CDR3, respectively, of SEQ ID NO:144. In one embodiment, the second binding domain binds to BCMA. In some embodiments, the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:143; and (ii) a VL comprising a VL CDR1, a VL CDR2, and a VL CDR3 having amino acid sequences of the VL CDR1, VL CDR2, and VL CDR3, respectively, of SEQ ID NO:144. In some embodiments, the second binding domain that binds to BCMA has a VH CDR1, VH CDR2, VH CDR3, VL CDR1, VL CDR2, and VL CDR3 having the amino acid sequence of SEQ ID NOs:137, 138, 139, 140, 141, and 142, respectively. In some embodiments, the second binding domain that binds to BCMA has a VH CDR1, VH CDR2, VH CDR3, VL CDR1, VL CDR2, and VL CDR3 having the amino acid sequence of SEQ ID NOs:664, 665, 666, 667, 668, and 669, respectively. In some embodiments, the second binding domain that binds to BCMA has a VH CDR1, VH CDR2, VH CDR3, VL CDR1, VL CDR2, and VL CDR3 having the amino acid sequence of SEQ ID NOs:670, 671, 672, 673, 674, and 675, respectively. In some embodiments, the second binding domain that binds to BCMA has a VH CDR1, VH CDR2, VH CDR3, VL CDR1, VL CDR2, and VL CDR3 having the amino acid sequence of SEQ ID NOs:676, 677, 678, 679, 680, and 681, respectively. In some embodiments, the second binding domain that binds to BCMA has a VH CDR1, VH CDR2, VH CDR3, VL CDR1, VL CDR2, and VL CDR3 having the amino acid sequence of SEQ ID NOs:682, 683, 684, 685, 686, and 687, respectively. In some embodiments, the second binding domain that binds to BCMA has a VH CDR1, VH CDR2, VH CDR3, VL CDR1, VL CDR2, and VL CDR3 having the amino acid sequence of SEQ ID NOs:688, 689, 690, 691, 692, and 693, respectively. In some embodiments, the second binding domain that binds to BCMA has a VH CDR1, VH CDR2, VH CDR3, VL CDR1, VL CDR2, and VL CDR3 having the amino acid sequence of SEQ ID NOs:694, 695, 696, 697, 698, and 699, respectively. In certain embodiments, the second binding domain that binds BCMA has a VH having the amino acid sequence of SEQ ID NO:143. In certain embodiments, the second binding domain that binds BCMA has a VL having the amino acid sequence of SEQ ID NO:144. In some embodiments, the second binding domain that binds BCMA has a VH having the amino acid sequence of SEQ ID NO:143, and a VL having the amino acid sequence of SEQ ID NO:144. In certain embodiments, the second binding domain that binds BCMA has a heavy chain having the amino acid sequence of SEQ ID NO:146. In certain embodiments, the second binding domain that binds BCMA has a light chain having the amino acid sequence of SEQ ID NO:147. In some embodiments, the second binding domain that binds BCMA has a heavy chain having the amino acid sequence of SEQ ID NO:146, and a light chain having the amino acid sequence of SEQ ID NO:147. In certain embodiments, the second binding domain that binds BCMA has an amino acid sequence of SEQ ID NO:145. In certain embodiments, the second binding domain that binds BCMA has an amino acid sequence of SEQ ID NO:148. In certain embodiments, the second binding domain that binds BCMA has an amino acid sequence of SEQ ID NO:149. In certain embodiments, the second binding domain that binds BCMA has a VH having at least 95% sequence identity to an amino acid sequence of SEQ ID NO:143. In certain embodiments, the second binding domain that binds BCMA has a VL having at least 95% sequence identity to an amino acid sequence of SEQ ID NO:144. In some embodiments, the second binding domain that binds BCMA has a VH having at least 95% sequence identity to an amino acid sequence of SEQ ID NO:143, and a VL having at least 95% sequence identity to an amino acid sequence of SEQ ID NO:144. In certain embodiments, the second binding domain that binds BCMA has a heavy chain having at least 95% sequence identity to an amino acid sequence of SEQ ID NO:146. In certain embodiments, the second binding domain that binds BCMA has a light chain having at least 95% sequence identity to an amino acid sequence of SEQ ID NO:147. In some embodiments, the second binding domain that binds BCMA has a heavy chain having at least 95% sequence identity to an amino acid sequence of SEQ ID NO:146, and a light chain having at least 95% sequence identity to an amino acid sequence of SEQ ID NO:147. In certain embodiments, the second binding domain that binds BCMA has an amino acid sequence having at least 95% sequence identity to an amino acid sequence of SEQ ID NO:145. In certain embodiments, the second binding domain that binds BCMA has an amino acid sequence having at least 95% sequence identity to an amino acid sequence of SEQ ID NO:148. In certain embodiments, the second binding domain that binds BCMA has an amino acid sequence having at least 95% sequence identity to an amino acid sequence of SEQ ID NO:149.

The TRGV9×BCMA multispecific antibody can comprise a first binding domain comprising any TRGV9 antibody provided herein. The TRGV9×BCMA multispecific antibody can further comprise a second binding domain comprising any BCMA antibody, including any BCMA antibody provided herein.

In some embodiments of the multispecific TRGV9 antibodies provided herein, the second target is PSMA. In one embodiment, the second binding domain binds to PSMA.

In some embodiments, the second binding domain that binds PSMA comprises a VH comprising a VH CDR1, a VH CDR2, and a VH CDR3 having amino acid sequences of the VH CDR1, VH CDR2, and VH CDR3, respectively, of SEQ ID NO:775. In some embodiments, the second binding domain that binds PSMA comprises a VL comprising a VL CDR1, a VL CDR2, and a VL CDR3 having amino acid sequences of the VL CDR1, VL CDR2, and VL CDR3, respectively, of SEQ ID NO:776. In some embodiments, the second binding domain that binds PSMA comprises: (i) a VH comprising a VH CDR1, a VH CDR2, and a VH CDR3 having amino acid sequences of the VH CDR1, VH CDR2, and VH CDR3, respectively, of SEQ ID NO:775; and (ii) a VL comprising a VL CDR1, a VL CDR2, and a VL CDR3 having amino acid sequences of the VL CDR1, VL CDR2, and VL CDR3, respectively, of SEQ ID NO:776. In some embodiments, the second binding domain that binds to PSMA has a VH CDR1, VH CDR2, VH CDR3, VL CDR1, VL CDR2, and VL CDR3 having the amino acid sequence of SEQ ID NOs:783, 784, 785, 786, 787 and 788, respectively. In certain embodiments, the second binding domain that binds PSMA has a VH having the amino acid sequence of SEQ ID NO:775. In certain embodiments, the second binding domain that binds PSMA has a VL having the amino acid sequence of SEQ ID NO:776. In some embodiments, the second binding domain that binds PSMA has a VH having the amino acid sequence of SEQ ID NO:775, and a VL having the amino acid sequence of SEQ ID NO:776. In certain embodiments, the second binding domain that binds PSMA has a heavy chain having the amino acid sequence of SEQ ID NO:781. In certain embodiments, the second binding domain that binds PSMA has a light chain having the amino acid sequence of SEQ ID NO:782. In some embodiments, the second binding domain that binds PSMA has a heavy chain having the amino acid sequence of SEQ ID NO:781, and a light chain having the amino acid sequence of SEQ ID NO:782. In certain embodiments, the second binding domain that binds PSMA has a VH having at least 95% sequence identity to an amino acid sequence of SEQ ID NO:775. In certain embodiments, the second binding domain that binds PSMA has a VL having at least 95% sequence identity to an amino acid sequence of SEQ ID NO:776. In some embodiments, the second binding domain that binds PSMA has a VH having at least 95% sequence identity to an amino acid sequence of SEQ ID NO:775, and a VL having at least 95% sequence identity to an amino acid sequence of SEQ ID NO:776. In certain embodiments, the second binding domain that binds PSMA has a heavy chain having at least 95% sequence identity to an amino acid sequence of SEQ ID NO:781. In certain embodiments, the second binding domain that binds PSMA has a light chain having at least 95% sequence identity to an amino acid sequence of SEQ ID NO:782. In some embodiments, the second binding domain that binds PSMA has a heavy chain having at least 95% sequence identity to an amino acid sequence of SEQ ID NO:781, and a light chain having at least 95% sequence identity to an amino acid sequence of SEQ ID NO:782.

The TRGV9×PSMA multispecific antibody can comprise a first binding domain comprising any TRGV9 antibody provided herein. The TRGV9×PSMA multispecific antibody can further comprise a second binding domain comprising any PSMA antibody, including any PSMA antibody provided herein.

In some embodiments, the VH CDR1, VH CDR2 and VH CDR3 amino acid sequences of the second binding domain that binds TRGV9 are according to the Kabat numbering system. In some embodiments, the VH CDR1, VH CDR2 and VH CDR3 amino acid sequences of the second binding domain that binds TRGV9 are according to the Chothia numbering system. In some embodiments, the VH CDR1, VH CDR2 and VH CDR3 amino acid sequences of the second binding domain that binds TRGV9 are according to the AbM numbering system. In some embodiments, the VH CDR1, VH CDR2 and VH CDR3 amino acid sequences of the second binding domain that binds TRGV9 are according to the Contact numbering system. In some embodiments, the VH CDR1, VH CDR2 and VH CDR3 amino acid sequences of the second binding domain that binds TRGV9 are according to the IMGT numbering system. In some embodiments, the VH CDR1, VH CDR2 and VH CDR3 amino acid sequences of the second binding domain that binds TRGV9 are according to the Exemplary numbering system. In some embodiments, the VL CDR1, VL CDR2 and VL CDR3 amino acid sequences of the second binding domain that binds TRGV9 are according to the Kabat numbering system. In some embodiments, the VL CDR1, VL CDR2 and VL CDR3 amino acid sequences of the second binding domain that binds TRGV9 are according to the Chothia numbering system. In some embodiments, the VL CDR1, VL CDR2 and VL CDR3 amino acid sequences of the second binding domain that binds TRGV9 are according to the AbM numbering system. In some embodiments, the VL CDR1, VL CDR2 and VL CDR3 amino acid sequences of the second binding domain that binds TRGV9 are according to the Contact numbering system. In some embodiments, the VL CDR1, VL CDR2 and VL CDR3 amino acid sequences of the second binding domain that binds TRGV9 are according to the IMGT numbering system. In some embodiments, the VL CDR1, VL CDR2 and VL CDR3 amino acid sequences of the second binding domain that binds TRGV9 are according to the Exemplary numbering system. In some embodiments, the VH CDR1, VH CDR2, VH CDR3, VL CDR1, VL CDR2, and VL CDR3 amino acid sequences of the second binding domain that binds the second target are according to the Kabat numbering system. In some embodiments, the VH CDR1, VH CDR2, VH CDR3, VL CDR1, VL CDR2, and VL CDR3 amino acid sequences of the second binding domain that binds the second target are according to the Chothia numbering system. In some embodiments, the VH CDR1, VH CDR2, VH CDR3, VL CDR1, VL CDR2, and VL CDR3 amino acid sequences of the second binding domain that binds the second target are according to the AbM numbering system. In some embodiments, the VH CDR1, VH CDR2, VH CDR3, VL CDR1, VL CDR2, and VL CDR3 amino acid sequences of the second binding domain that binds the second target are according to the Contact numbering system. In some embodiments, the VH CDR1, VH CDR2, VH CDR3, VL CDR1, VL CDR2, and VL CDR3 amino acid sequences of the second binding domain that binds the second target are according to the IMGT numbering system. In some embodiments, the VH CDR1, VH CDR2, VH CDR3, VL CDR1, VL CDR2, and VL CDR3 amino acid sequences of the second binding domain that binds the second target are according to the Exemplary numbering system.

In some embodiments, the multispecific TRGV9 antibody does not comprise L7A5_1 (TRGV9_1). In some embodiments, the multispecific TRGV9 antibody does not comprise L7A5_2 (TRGV9_2). In some embodiments, the multispecific TRGV9 antibody does not comprise L7A5_3 (TRGV9_3). In some embodiments, the multispecific TRGV9 antibody does not comprise L7A5_4 (TRGV9_4). In some embodiments, the multispecific TRGV9 antibody does not comprise a VH CDR1-3 or a VL CDR1-3 of L7A5_1 (TRGV9_1). In some embodiments, the multispecific TRGV9 antibody does not comprise a VH CDR1-3 or a VL CDR1-3 of L7A5_2 (TRGV9_2). In some embodiments, the multispecific TRGV9 antibody does not comprise a VH CDR1-3 or a VL CDR1-3 of L7A5_3 (TRGV9_3). In some embodiments, the multispecific TRGV9 antibody does not comprise a VH CDR1-3 or a VL CDR1-3 of L7A5_4 (TRGV9_4). In some embodiments, the multispecific TRGV9 antibody does not comprise a second binding arm that binds CD123.

In some embodiments, the second binding domain binds an antigen of the second target. In some embodiments, In some embodiments, the second binding domain binds an epitope of the second target. In some embodiments, the second binding domain specifically binds to the second target. In some embodiments, the second binding domain specifically binds an antigen of the second target. In some embodiments, the second binding domain specifically binds an epitope of the second target. In some embodiments, the VH CDR1, VH CDR2, VH CDR3, VL CDR1, VL CDR2 and VL CDR3 of the second binding domain form a binding site for an antigen of the second target. In some embodiments, the VH CDR1, VH CDR2, VH CDR3, VL CDR1, VL CDR2 and VL CDR3 of the second binding domain form a binding site for an epitope of the second target. In some embodiments, the VH region and the VL region of the second binding domain form a binding site for an antigen of the second target. In some embodiments, the VH region and the VL region of the second binding domain form a binding site for an epitope of the second target. In some embodiments, the heavy chain and the light chain of the second binding domain form a binding site for an antigen of the second target. In some embodiments, the heavy chain and the light chain of the second binding domain form a binding site for an epitope of the second target.

In some embodiments, the first binding domain of the multispecific TRGV9 antibody is multivalent. In some embodiments, the first binding domain of the multispecific TRGV9 antibody is capable of binding at least three antigens. In some embodiments, the first binding domain of the multispecific TRGV9 antibody is capable of binding at least four antigens. In some embodiments, the first binding domain of the multispecific TRGV9 antibody is capable of binding at least five antigens. In some embodiments, the second binding domain of the multispecific TRGV9 antibody is multivalent. In some embodiments, the second binding domain of the multispecific TRGV9 antibody is capable of binding at least three antigens. In some embodiments, the second binding domain of the multispecific TRGV9 antibody is capable of binding at least four antigens. In some embodiments, the second binding domain of the multispecific TRGV9 antibody is capable of binding at least five antigens.

In some embodiments of the multispecific TRGV9 antibodies provided herein, the second target is expressed by target cell. In some embodiments of the multispecific TRGV9 antibodies provided herein, the second target is on the surface of a target cell. In a specific embodiment, the target cell is an undesired cell. In some embodiments, the target cell expressing the second target is killed when the multispecific TRGV9 antibody binds to TRGV9 on the surface of a T cell and the second target. In a specific embodiment, the T cell is a γδ T cell.

In some embodiments, a first heavy chain (HC1) comprises a VH CDR1-3 that binds to TRGV9. In some embodiments, a first light chain (LC1) comprises a VL CDR1-3 that binds to TRGV9. In some embodiments, the HC1 VH CDR1-3 and the LC1 VL CDR1-3 form a binding site for the TRGV9. In certain embodiments, the binding site specifically binds TRGV9. In some embodiments, a second heavy chain (HC2) comprises a VH CDR1-3 that binds to the second target. In some embodiments, a second light chain (LC2) comprises a VL CDR1-3 that binds to the second target. In some embodiments, the HC2 VH CDR1-3 and the LC2 VL CDR1-3 form a binding site for the second target. In certain embodiments, the binding site specifically binds the second target. In certain embodiments, the TRGV9 is on the surface of a γδ T cell. In certain embodiments, the second target antigen is on the surface of a second target cell.

In one embodiment, the target cell is a cancer cell. In one embodiment, the target cell is a T cell. In one embodiment, the target cell is a B cell. In one embodiment, the target cell is a dendritic cell. In one embodiment, the target cell is a NK cell. In one embodiment, the target cell is a stem cell. In one embodiment, the target cell is a stem cell precursor. In one embodiment, the target cell is a monocyte. In one embodiment, the target cell is a macrophage. In one embodiment, the target cell is a granulocyte. In one embodiment, the target cell is a platelet. In one embodiment, the target cell is an erythrocyte. In one embodiment, the target cell is an endothelial cell. In one embodiment, the target cell is an epithelial cell. In one embodiment, the target cell is a pathogen. In one embodiment, the target cell is a blood cell. In one embodiment, the target cell is a myeloid cell.

In one embodiment, the second binding arm binds a second target. In one embodiment, the second target is present on a target cell. In one embodiment, the second target is present on the surface of a target cell. In certain embodiments, the target cell is a cancer cell. In a specific embodiment, the second target is a cancer antigen.

In some embodiments, the second target is on a cancer cell. In some embodiments, the target cell is a cancer cell. In a specific embodiment, the second target is on the surface of a cancer cell. In certain embodiments, the second target is an antigen on the surface of a cancer cell. In some embodiments, the antigen on the surface of the cancer cell is a tumor-specific antigen, a tumor-associated antigen, or a neoantigen.

In another aspect, provided herein is a multispecific antibody comprising: (a) a first binding domain that binds to TRGV9, and (b) a second binding domain that binds to a cancer antigen present on the surface of a cancer cell. In some embodiments, the antigen on the surface of the cancer cell is a tumor-specific antigen. In some embodiments, the antigen on the surface of the cancer cell is a tumor associated antigen. In some embodiments, the antigen on the surface of the cancer cell is a neoantigen. In certain embodiments, the first binding domain of the bispecific antibody specifically binds TRGV9. In some embodiments, the TRGV9 is present on the surface of a γδ T cell. In some embodiments, the cancer cell is killed when the multispecific antibody binds to the TRGV9 on the surface of the γδ T cell and the antigen on the surface of the cancer cell. Bispecific antibodies comprising any of the TRGV9 antibodies provided herein as the first binding domain are contemplated, in certain embodiments.

In some embodiments, the cancer antigen is CD123. In some embodiments, the cancer antigen is CD33. In some embodiments, the cancer antigen is BCMA. In some embodiments, the cancer antigen is PSMA. The binding of the TRGV9 multispecific antibody to TRGV9 present on the surface of the T cell, and the binding of the cancer antigen present on the surface of the cancer cell can, for example, result in the killing of the cancer cell.

In certain embodiments, the anti-TRGV9 antibodies or antigen binding fragments thereof binds to a first epitope located on TRGV9 and a second epitope of a cancer cell.

In some embodiments, provided herein is a bispecific antibody comprising: (a) a first binding domain that binds to a TRGV9 antigen, and (b) a second binding domain that binds to a cancer cell antigen. In some embodiments, provided herein is a bispecific antibody comprising: (a) a first binding domain that specifically binds to a TRGV9 antigen, and (b) a second binding domain that specifically binds to a cancer cell antigen. In some embodiments, provided herein is a bispecific antibody comprising: (a) a first binding domain that binds to a first epitope on a TRGV9 antigen, and (b) a second binding domain that binds to a second epitope on a cancer cell antigen. In some embodiments, provided herein is a bispecific antibody comprising: (a) a first binding domain that specifically binds to a first epitope on a TRGV9 antigen, and (b) a second binding domain that specifically binds to a second epitope on a cancer cell antigen. In some embodiments, the antigen is CD123. In some embodiments, the antigen is CD33. In some embodiments, the antigen is BCMA. In some embodiments, the antigen is PSMA.

In an embodiment of the bispecific antibodies provided herein, the first epitope is located on TRGV9 and the second epitope is located on the surface of a cancer cell. In some embodiments, the second epitope is located on a cancer cell antigen. In an embodiment of the bispecific antibodies provided herein, the first epitope is located on TRGV9 and the second epitope is located on a tumor. In an embodiment of the bispecific antibodies provided herein, the first epitope is located on TRGV9 and the second epitope is located on a tumor-specific antigen. In an embodiment of the bispecific antibodies provided herein, the first epitope is located on TRGV9 and the second epitope is located on a tumor associated antigen. In an embodiment of the bispecific antibodies provided herein, the first epitope is located on TRGV9 and the second epitope is located on a neoantigen.

In some embodiments, the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer. In some embodiments, the cancer is a adrenal cancer. In some embodiments, the cancer is a anal cancer. In some embodiments, the cancer is an appendix cancer. In some embodiments, the cancer is a bile duct cancer. In some embodiments, the cancer is a bladder cancer. In some embodiments, the cancer is a bone cancer. In some embodiments, the cancer is a brain cancer. In some embodiments, the cancer is a breast cancer. In some embodiments, the cancer is a cervical cancer. In some embodiments, the cancer is a colorectal cancer. In some embodiments, the cancer is a esophageal cancer. In some embodiments, the cancer is a gallbladder cancer. In some embodiments, the cancer is a gestational trophoblastic. In some embodiments, the cancer is a head and neck cancer. In some embodiments, the cancer is a Hodgkin lymphoma. In some embodiments, the cancer is an intestinal cancer. In some embodiments, the cancer is a kidney cancer. In some embodiments, the cancer is a leukemia. In some embodiments, the cancer is a liver cancer. In some embodiments, the cancer is a lung cancer. In some embodiments, the cancer is a melanoma. In some embodiments, the cancer is a mesothelioma. In some embodiments, the cancer is a multiple myeloma. In some embodiments, the cancer is a neuroendocrine tumor. In some embodiments, the cancer is a non-Hodgkin lymphoma. In some embodiments, the cancer is an oral cancer. In some embodiments, the cancer is a ovarian cancer. In some embodiments, the cancer is a pancreatic cancer. In some embodiments, the cancer is a prostate cancer. In some embodiments, the cancer is a sinus cancer. In some embodiments, the cancer is a skin cancer. In some embodiments, the cancer is a soft tissue sarcoma spinal cancer. In some embodiments, the cancer is a stomach cancer. In some embodiments, the cancer is a testicular cancer. In some embodiments, the cancer is a throat cancer. In some embodiments, the cancer is a thyroid cancer. In some embodiments, the cancer is a uterine cancer endometrial cancer. In some embodiments, the cancer is a vaginal cancer. In some embodiments, the cancer is a vulvar cancer.

In some embodiments, the adrenal cancer is an adrenocortical carcinoma (ACC), adrenal cortex cancer, pheochromocytoma, or neuroblastoma.

In some embodiments, the anal cancer is a squamous cell carcinoma, cloacogenic carcinoma, adenocarcinoma, basal cell carcinoma, or melanoma.

In some embodiments, the appendix cancer is a neuroendocrine tumor (NET), mucinous adenocarcinoma, goblet cell carcinoid, intestinal-type adenocarcinoma, or signet-ring cell adenocarcinoma.

In some embodiments, the bile duct cancer is an extrahepatic bile duct cancer, adenocarcinomas, hilar bile duct cancer, perihilar bile duct cancer, distal bile duct cancer, or intrahepatic bile duct cancer.

In some embodiments, the bladder cancer is transitional cell carcinoma (TCC), papillary carcinoma, flat carcinoma, squamous cell carcinoma, adenocarcinoma, small-cell carcinoma, or sarcoma.

In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer.

In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma.

In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer.

In some embodiments, the cervical cancer is a squamous cell carcinoma, or adenocarcinoma.

In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma.

In some embodiments, the esophageal cancer is an adenocarcinoma or squamous cell carcinoma.

In some embodiments, the gall bladder cancer is an adenocarcinoma, papillary adenocarcinoma, adenosquamous carcinoma, squamous cell carcinoma, small cell carcinoma, or sarcoma.

In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT).

In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer.

In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL).

In some embodiments, the intestinal cancer is a small intestine cancer, small bowel cancer, adenocarcinoma, sarcoma, gastrointestinal stromal tumors, carcinoid tumors, or lymphoma.

In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma.

In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML.

In some embodiments, the liver cancer is a hepatocellular carcinoma (HCC), fibrolamellar HCC, cholangiocarcinoma, angiosarcoma, or liver metastasis.

In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor.

In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma.

In some embodiments, the mesothelioma is a pleural mesothelioma, peritoneal mesothelioma, pericardial mesothelioma, or testicular mesothelioma.

In some embodiments, the multiple myeloma is an active myeloma or smoldering myeloma.

In some embodiments, the neuroendocrine tumor, is a gastrointestinal neuroendocrine tumor, pancreatic neuroendocrine tumor, or lung neuroendocrine tumor.

In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma.

In a specific embodiment, the cancer is multiple myeloma (MM). In another specific embodiment, the cancer is chronic lymphocytic leukemia. In other embodiments, the cancer is acute B-lymphoblastic leukemia. In yet other embodiments, the cancer is non-Hodgkin lymphoma (NHL). In some embodiments, the cancer is Hodgkin lymphoma.

In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer.

In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst.

In some embodiments, the pancreatic cancer is a pancreatic exocrine gland cancer, pancreatic endocrine gland cancer, or pancreatic adenocarcinoma, islet cell tumor, or neuroendocrine tumor.

In some embodiments, the prostate cancer is a prostate adenocarcinoma, prostate sarcoma, transitional cell carcinoma, small cell carcinoma, or neuroendocrine tumor.

In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer.

In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma.

In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma.

In some embodiments, the spinal cancer is a spinal metastatic tumor.

In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid.

In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor.

In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer.

In some embodiments, the thyroid cancer is a papillary carcinoma, follicular carcinoma, Hürthle cell carcinoma, medullary thyroid carcinoma, or anaplastic carcinoma.

In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma.

In some embodiments, the vaginal cancer is a squamous cell carcinoma, adenocarcinoma, melanoma, or sarcoma.

In some embodiments, the vulvar cancer is a squamous cell carcinoma or adenocarcinoma.

In one embodiment, the cancer is a solid cancer. In one embodiment, the cancer is a solid tumor. In one embodiment, the cancer is a liquid cancer. In one embodiment, the cancer is a liquid tumor. In some embodiments, the cancer is a hematologic malignancy. In cersin embodiments, the cancer is benign. In some embodiments, the cancer is malignant. Im some embodiments, the cancer is metastatic.

In some embodiments, the second epitope is located on a cancer antigen.

In some embodiments, the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, prostatic acid phosphatase (PAP), PDGFRα, prostate-specific antigen (PSA), PSA3, prostate-specific membrane antigen (PSMA), RANKL, SLAMF7, STEAP1, T cell receptor gamma alternate reading frame protein (TARP), TROP2, VEGF, or VEGF-R. In some embodiments, the cancer antigen is angiopoietin. In some embodiments, the cancer antigen is BCMA. In some embodiments, the cancer antigen is CD19. In some embodiments, the cancer antigen is CD20. In some embodiments, the cancer antigen is CD22. In some embodiments, the cancer antigen is CD25 (IL2-R). In some embodiments, the cancer antigen is CD30. In some embodiments, the cancer antigen is CD33. In some embodiments, the cancer antigen is CD37. In some embodiments, the cancer antigen is CD38. In some embodiments, the cancer antigen is CD52. In some embodiments, the cancer antigen is CD56. In some embodiments, the cancer antigen is CD123 (IL-3R). In some embodiments, the cancer antigen is cMET. In some embodiments, the cancer antigen is DLL/Notch. In some embodiments, the cancer antigen is EGFR. In some embodiments, the cancer antigen is EpCAM. In some embodiments, the cancer antigen is FGF. In some embodiments, the cancer antigen is FGF-R. In some embodiments, the cancer antigen is GD2. In some embodiments, the cancer antigen is HER2. In some embodiments, the cancer antigen is Mesothelin. In some embodiments, the cancer antigen is Nectin-4. In some embodiments, the cancer antigen is PAP. In some embodiments, the cancer antigen is PDGFRα. In some embodiments, the cancer antigen is PSA. In some embodiments, the cancer antigen is PSA3. In some embodiments, the cancer antigen is PSCA. In some embodiments, the cancer antigen is PSMA. In some embodiments, the cancer antigen is RANKL. In some embodiments, the cancer antigen is SLAMF7. In some embodiments, the cancer antigen is STEAP1. In some embodiments, the cancer antigen is TARP. In some embodiments, the cancer antigen is TROP2. In some embodiments, the cancer antigen is VEGF. In some embodiments, the cancer antigen is VEGF-R.

In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1. In some embodiments, the cancer antigen is CEA. In some embodiments, the cancer antigen is immature laminin receptor. In some embodiments, the cancer antigen is TAG-72. In some embodiments, the cancer antigen is HPV E6. In some embodiments, the cancer antigen is HPV E7. In some embodiments, the cancer antigen is BING-4. In some embodiments, the cancer antigen is calcium-activated chloride channel 2. In some embodiments, the cancer antigen is cyclin-B1. In some embodiments, the cancer antigen is 9D7. In some embodiments, the cancer antigen is EpCAM. In some embodiments, the cancer antigen is EphA3. In some embodiments, the cancer antigen is Her2/neu. In some embodiments, the cancer antigen is telomerase. In some embodiments, the cancer antigen is mesothelin. In some embodiments, the cancer antigen is SAP-1. In some embodiments, the cancer antigen is surviving. In some embodiments, the cancer antigen is a BAGE family antigen. In some embodiments, the cancer antigen is CAGE family antigen. In some embodiments, the cancer antigen is GAGE family antigen. In some embodiments, the cancer antigen is MAGE family antigen. In some embodiments, the cancer antigen is SAGE family antigen. In some embodiments, the cancer antigen is XAGE family antigen. In some embodiments, the cancer antigen is NY-ESO-1/LAGE-1. In some embodiments, the cancer antigen is PRAME. In some embodiments, the cancer antigen is SSX-2. In some embodiments, the cancer antigen is Melan-A. In some embodiments, the cancer antigen is MART-1. In some embodiments, the cancer antigen is Gp100. In some embodiments, the cancer antigen is pmel17. In some embodiments, the cancer antigen is tyrosinase. In some embodiments, the cancer antigen is TRP-1. In some embodiments, the cancer antigen is TRP-2. In some embodiments, the cancer antigen is P. polypeptide. In some embodiments, the cancer antigen is MC1R. In some embodiments, the cancer antigen is prostate-specific antigen. In some embodiments, the cancer antigen is β-catenin. In some embodiments, the cancer antigen is BRCA1. In some embodiments, the cancer antigen is BRCA2. In some embodiments, the cancer antigen is CDK4. In some embodiments, the cancer antigen is CML66. In some embodiments, the cancer antigen is fibronectin. In some embodiments, the cancer antigen is MART-2. In some embodiments, the cancer antigen is p53. In some embodiments, the cancer antigen is Ras. In some embodiments, the cancer antigen is TGF-βRII. In some embodiments, the cancer antigen is MUC1.

The binding of the TRGV9 bispecific antibody to TRGV9 present on the surface of the γδ T cell, and the binding of the tumor associated antigen present on the surface of the cancer cell can, for example, result in the killing of the cancer cell.

In an embodiment of the bispecific antibodies provided herein, the first epitope is located on TRGV9 and the second epitope is located on CD123. In an embodiment of the bispecific antibodies provided herein, the first epitope is located on TRGV9 and the second epitope is located on PD-1, PD-L1, CTLA-4, EGFR, HER-2, CD19, CD20, CD3 and/or other cancer associated immune suppressors or surface antigens.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is a T cell. In a specific embodiment, the second target is a T cell antigen. In some embodiments a multispecific TRGV9 antibody provided herein comprises: (a) a first binding domain that binds to TRGV9, and (b) a second binding domain that binds to a T cell antigen present on the surface of a T cell. In certain embodiments, the first binding domain of the multispecific TRGV9 antibody specifically binds TRGV9. In some embodiments, the TRGV9 is present on the surface of a γδ T cell. In some embodiments, the T cell is killed when the multispecific antibody binds to the TRGV9 on the surface of the γδ T cell and the antigen on the surface of the T cell. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of a T cell. In some embodiments, provided herein is a bispecific antibody comprising: (a) a first binding domain that binds to a TRGV9 antigen, and (b) a second binding domain that binds to a T cell antigen. In some embodiments, provided herein is a bispecific antibody comprising: (a) a first binding domain that specifically binds to a TRGV9 antigen, and (b) a second binding domain that specifically binds to a T cell antigen. In some embodiments, provided herein is a bispecific antibody comprising: (a) a first binding domain that binds to a first epitope on a TRGV9 antigen, and (b) a second binding domain that binds to a second epitope on a T cell antigen. In some embodiments, provided herein is a bispecific antibody comprising: (a) a first binding domain that specifically binds to a first epitope on a TRGV9 antigen, and (b) a second binding domain that specifically binds to a second epitope on a T cell antigen. In an embodiment of the bispecific antibodies provided herein, the first epitope is located on TRGV9 and the second epitope is located on the surface of a T cell. In some embodiments, the second epitope is located on a T cell antigen. In a specific embodiment, the T cell antigen is not a TRGV9 antigen.

In some embodiments, the T cell antigen is a CD13, CD16, CD17, CD18, CD20, CD21, CD23, CD25, CD26, CD27, CD28, CD29, CD30, CD31, CD32b, CD35, CD37, CD38, CD39, CD43, CD44, CD45, CD45RA, CD45RB, CD45RC, CD45RO, CD46, CD47, CD48, CD49a, CD49b, CD49c, CD49d, CD49e, CD49f, CD50, CD52, CD53, CD54, CD55, CD56, CD57, CD58, CD59, CD60a, CD62L, CD63, CD68, CD69, CD70, CD71, CD73, CD74, CD75S, CD80, CD81, CD82, CD84, CD85A, CD85J, CD86, CD87, CD92, CD94, CD95, CD96, CD97, CD98, CD99, CD99R, CD100, CD101, CD102, CD103, CD107a, CD107b, CD108, CD109, CD119, CD120a, CD120b, CD121a, CD121b, CD122, CD124, CD126, CD127, CD128, CD129, CD130, CD132, CD134, CD137, CD146, CD147, CD148, CD150, CD152, CD153, CD154, CD156b, CD158a, CD158b1, CD158b2, CD158e1/e2, CD158f, CD158g, CD158h, CD158i, CD158j, CD158k, CD159a, CD160, CD161, CD162, CD164, CD172g, CD178, CD181, CD182, CD183, CD184, CD185, CD186, CD191, CD192, CD193, CD194, CD195, CD196, CD197, CDw198, CDw199, CD205, CD210a, CDw210b, CD212, CD215, CD217, CD218a, CD218b, CD220, CD221, CD222, CD223, CD224, CD225, CD226, CD227, CD229, CD230, CD231, CD244, CD245, CD246, CD247, CD253, CD254, CD255, CD256, CD257, CD258, CD259, CD260, CD261, CD262, CD263, CD264, CD267, CD268, CD270, CD272, CD273, CD274, CD275, CD277, CD278, CD279, CD283, CD288, CD289, CD290, CD294, CD295, CD296, CD298, CD300a, CD300c, CD300e, CD305, CD306, CD307c, CD314, CD316, CD317, CD319, CD321, CD328, CD351, CD352, CD352, CD354, CD355, CD357, CD358, CD359, CD360, CD361, CD362, or CD363 antigen. In some embodiments, the T cell antigen is a CD3 antigen. In some embodiments, the T cell antigen is a CD4 antigen. In some embodiments, the T cell antigen is a CD8 antigen. In some embodiments, the T cell antigen is a CD13 antigen. In some embodiments, the T cell antigen is a CD16 antigen. In some embodiments, the T cell antigen is a CD17 antigen. In some embodiments, the T cell antigen is a CD18 antigen. In some embodiments, the T cell antigen is a CD19 antigen. In some embodiments, the T cell antigen is a CD20 antigen. In some embodiments, the T cell antigen is a CD21 antigen. In some embodiments, the T cell antigen is a CD23 antigen. In some embodiments, the T cell antigen is a CD25 antigen. In some embodiments, the T cell antigen is a CD26 antigen. In some embodiments, the T cell antigen is a CD27 antigen. In some embodiments, the T cell antigen is a CD28 antigen. In some embodiments, the T cell antigen is a CD29 antigen. In some embodiments, the T cell antigen is a CD30 antigen. In some embodiments, the T cell antigen is a CD31 antigen. In some embodiments, the T cell antigen is a CD32b antigen. In some embodiments, the T cell antigen is a CD35 antigen. In some embodiments, the T cell antigen is a CD37 antigen. In some embodiments, the T cell antigen is a CD38 antigen. In some embodiments, the T cell antigen is a CD39 antigen. In some embodiments, the T cell antigen is a CD43 antigen. In some embodiments, the T cell antigen is a CD44 antigen. In some embodiments, the T cell antigen is a CD45 antigen. In some embodiments, the T cell antigen is a CD45RA antigen. In some embodiments, the T cell antigen is a CD45RB antigen. In some embodiments, the T cell antigen is a CD45RC antigen. In some embodiments, the T cell antigen is a CD45RO antigen. In some embodiments, the T cell antigen is a CD46 antigen. In some embodiments, the T cell antigen is a CD47 antigen. In some embodiments, the T cell antigen is a CD48 antigen. In some embodiments, the T cell antigen is a CD49 antigen. In some embodiments, the T cell antigen is a CD49b antigen. In some embodiments, the T cell antigen is a CD49c antigen. In some embodiments, the T cell antigen is a CD49d antigen. In some embodiments, the T cell antigen is a CD49e antigen. In some embodiments, the T cell antigen is a CD49f antigen. In some embodiments, the T cell antigen is a CD50 antigen. In some embodiments, the T cell antigen is a CD52 antigen. In some embodiments, the T cell antigen is a CD53 antigen. In some embodiments, the T cell antigen is a CD54 antigen. In some embodiments, the T cell antigen is a CD55 antigen. In some embodiments, the T cell antigen is a CD56 antigen. In some embodiments, the T cell antigen is a CD57 antigen. In some embodiments, the T cell antigen is a CD58 antigen. In some embodiments, the T cell antigen is a CD59 antigen. In some embodiments, the T cell antigen is a CD60a antigen. In some embodiments, the T cell antigen is a CD62L antigen. In some embodiments, the T cell antigen is a CD63 antigen. In some embodiments, the T cell antigen is a CD68 antigen. In some embodiments, the T cell antigen is a CD69 antigen. In some embodiments, the T cell antigen is a CD70 antigen. In some embodiments, the T cell antigen is a CD71 antigen. In some embodiments, the T cell antigen is a CD73 antigen. In some embodiments, the T cell antigen is a CD74 antigen. In some embodiments, the T cell antigen is a CD75S antigen. In some embodiments, the T cell antigen is a CD80 antigen. In some embodiments, the T cell antigen is a CD81 antigen. In some embodiments, the T cell antigen is a CD82 antigen. In some embodiments, the T cell antigen is a CD84 antigen. In some embodiments, the T cell antigen is a CD85A antigen. In some embodiments, the T cell antigen is a CD85J antigen. In some embodiments, the T cell antigen is a CD86 antigen. In some embodiments, the T cell antigen is a CD87 antigen. In some embodiments, the T cell antigen is a CD92 antigen. In some embodiments, the T cell antigen is a CD94 antigen. In some embodiments, the T cell antigen is a CD95 antigen. In some embodiments, the T cell antigen is a CD96 antigen. In some embodiments, the T cell antigen is a CD97 antigen. In some embodiments, the T cell antigen is a CD98 antigen. In some embodiments, the T cell antigen is a CD99 antigen. In some embodiments, the T cell antigen is a CD99R antigen. In some embodiments, the T cell antigen is a CD100 antigen. In some embodiments, the T cell antigen is a CD101 antigen. In some embodiments, the T cell antigen is a CD102 antigen. In some embodiments, the T cell antigen is a CD103 antigen. In some embodiments, the T cell antigen is a CD107a antigen. In some embodiments, the T cell antigen is a CD107b antigen. In some embodiments, the T cell antigen is a CD108 antigen. In some embodiments, the T cell antigen is a CD109 antigen. In some embodiments, the T cell antigen is a CD119 antigen. In some embodiments, the T cell antigen is a CD120a antigen. In some embodiments, the T cell antigen is a CD120b antigen. In some embodiments, the T cell antigen is a CD121a antigen. In some embodiments, the T cell antigen is a CD121b antigen. In some embodiments, the T cell antigen is a CD122 antigen. In some embodiments, the T cell antigen is a CD124 antigen. In some embodiments, the T cell antigen is a CD126 antigen. In some embodiments, the T cell antigen is a CD127 antigen. In some embodiments, the T cell antigen is a CD128 antigen. In some embodiments, the T cell antigen is a CD129 antigen. In some embodiments, the T cell antigen is a CD130 antigen. In some embodiments, the T cell antigen is a CD132 antigen. In some embodiments, the T cell antigen is a CD134 antigen. In some embodiments, the T cell antigen is a CD137 antigen. In some embodiments, the T cell antigen is a CD146 antigen. In some embodiments, the T cell antigen is a CD147 antigen. In some embodiments, the T cell antigen is a CD148 antigen. In some embodiments, the T cell antigen is a CD150 antigen. In some embodiments, the T cell antigen is a CD152 antigen. In some embodiments, the T cell antigen is a CD153 antigen. In some embodiments, the T cell antigen is a CD154 antigen. In some embodiments, the T cell antigen is a CD156b antigen. In some embodiments, the T cell antigen is a CD158a antigen. In some embodiments, the T cell antigen is a CD158b1 antigen. In some embodiments, the T cell antigen is a CD158b2 antigen. In some embodiments, the T cell antigen is a CD158e1/e2 antigen. In some embodiments, the T cell antigen is a CD158f antigen. In some embodiments, the T cell antigen is a CD158g antigen. In some embodiments, the T cell antigen is a CD158h antigen. In some embodiments, the T cell antigen is a CD158h antigen. In some embodiments, the T cell antigen is a CD158i antigen. In some embodiments, the T cell antigen is a CD158j antigen. In some embodiments, the T cell antigen is a CD158k antigen. In some embodiments, the T cell antigen is a CD159a antigen. In some embodiments, the T cell antigen is a CD160 antigen. In some embodiments, the T cell antigen is a CD161 antigen. In some embodiments, the T cell antigen is a CD162 antigen. In some embodiments, the T cell antigen is a CD164 antigen. In some embodiments, the T cell antigen is a CD172g antigen. In some embodiments, the T cell antigen is a CD178 antigen. In some embodiments, the T cell antigen is a CD181 antigen. In some embodiments, the T cell antigen is a CD182 antigen. In some embodiments, the T cell antigen is a CD183 antigen. In some embodiments, the T cell antigen is a CD184 antigen. In some embodiments, the T cell antigen is a CD185 antigen. In some embodiments, the T cell antigen is a CD186 antigen. In some embodiments, the T cell antigen is a CD191 antigen. In some embodiments, the T cell antigen is a CD192 antigen. In some embodiments, the T cell antigen is a CD193 antigen. In some embodiments, the T cell antigen is a CD194 antigen. In some embodiments, the T cell antigen is a CD195 antigen. In some embodiments, the T cell antigen is a CD196 antigen. In some embodiments, the T cell antigen is a CD197 antigen. In some embodiments, the T cell antigen is a CDw198 antigen. In some embodiments, the T cell antigen is a CDw199 antigen. In some embodiments, the T cell antigen is a CD205 antigen. In some embodiments, the T cell antigen is a CD210a antigen. In some embodiments, the T cell antigen is a CDw210b antigen. In some embodiments, the T cell antigen is a CD212 antigen. In some embodiments, the T cell antigen is a CD215 antigen. In some embodiments, the T cell antigen is a CD217 antigen. In some embodiments, the T cell antigen is a CD218a antigen. In some embodiments, the T cell antigen is a CD218b antigen. In some embodiments, the T cell antigen is a CD220 antigen. In some embodiments, the T cell antigen is a CD221 antigen. In some embodiments, the T cell antigen is a CD222 antigen. In some embodiments, the T cell antigen is a CD223 antigen. In some embodiments, the T cell antigen is a CD224 antigen. In some embodiments, the T cell antigen is a CD225 antigen. In some embodiments, the T cell antigen is a CD226 antigen. In some embodiments, the T cell antigen is a CD227 antigen. In some embodiments, the T cell antigen is a CD229 antigen. In some embodiments, the T cell antigen is a CD230 antigen. In some embodiments, the T cell antigen is a CD231 antigen. In some embodiments, the T cell antigen is a CD244 antigen. In some embodiments, the T cell antigen is a CD245 antigen. In some embodiments, the T cell antigen is a CD246 antigen. In some embodiments, the T cell antigen is a CD247 antigen. In some embodiments, the T cell antigen is a CD253 antigen. In some embodiments, the T cell antigen is a CD254 antigen. In some embodiments, the T cell antigen is a CD255 antigen. In some embodiments, the T cell antigen is a CD256 antigen. In some embodiments, the T cell antigen is a CD257 antigen. In some embodiments, the T cell antigen is a CD258 antigen. In some embodiments, the T cell antigen is a CD259 antigen. In some embodiments, the T cell antigen is a CD260 antigen. In some embodiments, the T cell antigen is a CD261 antigen. In some embodiments, the T cell antigen is a CD262 antigen. In some embodiments, the T cell antigen is a CD263 antigen. In some embodiments, the T cell antigen is a CD264 antigen. In some embodiments, the T cell antigen is a CD267 antigen. In some embodiments, the T cell antigen is a CD268 antigen. In some embodiments, the T cell antigen is a CD270 antigen. In some embodiments, the T cell antigen is a CD272 antigen. In some embodiments, the T cell antigen is a CD273 antigen. In some embodiments, the T cell antigen is a CD274 antigen. In some embodiments, the T cell antigen is a CD275 antigen. In some embodiments, the T cell antigen is a CD277 antigen. In some embodiments, the T cell antigen is a CD278 antigen. In some embodiments, the T cell antigen is a CD279 antigen. In some embodiments, the T cell antigen is a CD283 antigen. In some embodiments, the T cell antigen is a CD288 antigen. In some embodiments, the T cell antigen is a CD289 antigen. In some embodiments, the T cell antigen is a CD290 antigen. In some embodiments, the T cell antigen is a CD294 antigen. In some embodiments, the T cell antigen is a CD295 antigen. In some embodiments, the T cell antigen is a CD296 antigen. In some embodiments, the T cell antigen is a CD298 antigen. In some embodiments, the T cell antigen is a CD300a antigen. In some embodiments, the T cell antigen is a CD300c antigen. In some embodiments, the T cell antigen is a CD300e antigen. In some embodiments, the T cell antigen is a CD305 antigen. In some embodiments, the T cell antigen is a CD306 antigen. In some embodiments, the T cell antigen is a CD307c antigen. In some embodiments, the T cell antigen is a CD314 antigen. In some embodiments, the T cell antigen is a CD316 antigen. In some embodiments, the T cell antigen is a CD317 antigen. In some embodiments, the T cell antigen is a CD319 antigen. In some embodiments, the T cell antigen is a CD321 antigen. In some embodiments, the T cell antigen is a CD328 antigen. In some embodiments, the T cell antigen is a CD351 antigen. In some embodiments, the T cell antigen is a CD352 antigen. In some embodiments, the T cell antigen is a CD352 antigen. In some embodiments, the T cell antigen is a CD354 antigen. In some embodiments, the T cell antigen is a CD355 antigen. In some embodiments, the T cell antigen is a CD357 antigen. In some embodiments, the T cell antigen is a CD358 antigen. In some embodiments, the T cell antigen is a CD359 antigen. In some embodiments, the T cell antigen is a CD360 antigen. In some embodiments, the T cell antigen is a CD361 antigen. In some embodiments, the T cell antigen is a CD362 antigen. In some embodiments, the T cell antigen is a CD363 antigen.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is a B cell. In a specific embodiment, the second target is a B cell antigen. In some embodiments a multispecific TRGV9 antibody provided herein comprises: (a) a first binding domain that binds to TRGV9, and (b) a second binding domain that binds to a B cell antigen present on the surface of a B cell. In certain embodiments, the first binding domain of the multispecific TRGV9 antibody specifically binds TRGV9. In some embodiments, the TRGV9 is present on the surface of a γδ T cell. In some embodiments, the B cell is killed when the multispecific antibody binds to the TRGV9 on the surface of the γδ T cell and the antigen on the surface of the B cell. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of a B cell. In some embodiments, provided herein is a bispecific antibody comprising: (a) a first binding domain that binds to a TRGV9 antigen, and (b) a second binding domain that binds to a B cell antigen. In some embodiments, provided herein is a bispecific antibody comprising: (a) a first binding domain that specifically binds to a TRGV9 antigen, and (b) a second binding domain that specifically binds to a B cell antigen. In some embodiments, provided herein is a bispecific antibody comprising: (a) a first binding domain that binds to a first epitope on a TRGV9 antigen, and (b) a second binding domain that binds to a second epitope on a B cell antigen. In some embodiments, provided herein is a bispecific antibody comprising: (a) a first binding domain that specifically binds to a first epitope on a TRGV9 antigen, and (b) a second binding domain that specifically binds to a second epitope on a B cell antigen. In an embodiment of the bispecific antibodies provided herein, the first epitope is located on TRGV9 and the second epitope is located on the surface of a B cell. In some embodiments, the second epitope is located on a B cell antigen.

In some embodiments, the B cell antigen is a CD1a, CD1b, CD1c, CD1d, CD2, CD5, CD6, CD9, CD11a, CD11b, CD11c, CD17, CD18, CD19, CD20, CD21, CD22, CD23, CD24, CD25, CD26, CD27, CD29, CD30, CD31, CD32a, CD32b, CD35, CD37, CD38, CD39, CD40, CD45, CD45RA, CD45RB, CD45RC, CD45RO, CD46, CD47, CD48, CD49b, CD49c, CD49d, CD50, CD52, CD53, CD54, CD55, CD58, CD60a, CD62L, CD63, CD68, CD69, CD70, CD72, CD73, CD74, CD75, CD75S, CD77, CD79a, CD79b, CD80, CD81, CD82, CD83, CD84, CD85E, CD851, CD85J, CD86, CD92, CD95, CD97, CD98, CD99, CD100, CD102, CD108, CD119, CD120a, CD120b, CD121b, CD122, CD124, CD125, CD126, CD130, CD132, CD137, CD138, CD139, CD147, CD148, CD150, CD152, CD162, CD164, CD166, CD167a, CD170, CD171, CD175, CD175s, CD180, CD184, CD185, CD192, CD196, CD197, CD200, CD205, CD201a, CDw210b, CD212, CD213a1, CD213a2, CD 215, CD217, CD218a, CD218b, CD220, CD221, CD222, CD224, CD225, CD226, CD227, CD229, CD230, CD232, CD252, CD252, CD254, CD255, CD256, CD257 CD258, CD259, CD260, CD261, CD262, CD263, CD264, CD267-270, CD272, CD274, CD275, CD277, CD279, CD283, CD289, CD290, CD295, CD298, CD300, CD300c, CD305, CD306, CD307a, CD307b, CD307c, CD307d, CD307e, CD314, CD215, CD316, CD317, CD319, CD321, CD327, CD328, CD329, CD338, CD351, CD352, CD353, CD354, CD355, CD356, CD357, CD358, CD360, CD361, CD362, or CD363 antigen. In some embodiments, the B cell antigen is a CD1a antigen. In some embodiments, the B cell antigen is a CD1b antigen. In some embodiments, the B cell antigen is a CD1c antigen. In some embodiments, the B cell antigen is a CD1d antigen. In some embodiments, the B cell antigen is a CD2 antigen. In some embodiments, the B cell antigen is a CD5 antigen. In some embodiments, the B cell antigen is a CD6 antigen. In some embodiments, the B cell antigen is a CD9 antigen. In some embodiments, the B cell antigen is a CD11a antigen. In some embodiments, the B cell antigen is a CD11b antigen. In some embodiments, the B cell antigen is a CD11c antigen. In some embodiments, the B cell antigen is a CD17 antigen. In some embodiments, the B cell antigen is a CD18 antigen. In some embodiments, the B cell antigen is a CD19 antigen. In some embodiments, the B cell antigen is a CD20 antigen. In some embodiments, the B cell antigen is a CD21 antigen. In some embodiments, the B cell antigen is a CD22 antigen. In some embodiments, the B cell antigen is a CD23 antigen. In some embodiments, the B cell antigen is a CD24 antigen. In some embodiments, the B cell antigen is a CD25 antigen. In some embodiments, the B cell antigen is a CD26 antigen. In some embodiments, the B cell antigen is a CD27 antigen. In some embodiments, the B cell antigen is a CD29 antigen. In some embodiments, the B cell antigen is a CD30 antigen. In some embodiments, the B cell antigen is a CD31 antigen. In some embodiments, the B cell antigen is a CD32a antigen. In some embodiments, the B cell antigen is a CD32b antigen. In some embodiments, the B cell antigen is a CD35 antigen. In some embodiments, the B cell antigen is a CD37 antigen. In some embodiments, the B cell antigen is a CD38 antigen. In some embodiments, the B cell antigen is a CD39 antigen. In some embodiments, the B cell antigen is a CD40 antigen. In some embodiments, the B cell antigen is a CD45 antigen. In some embodiments, the B cell antigen is a CD45RA antigen. In some embodiments, the B cell antigen is a CD45RB antigen. In some embodiments, the B cell antigen is a CD45RC antigen. In some embodiments, the B cell antigen is a CD45RO antigen. In some embodiments, the B cell antigen is a CD46 antigen. In some embodiments, the B cell antigen is a CD47 antigen. In some embodiments, the B cell antigen is a CD48 antigen. In some embodiments, the B cell antigen is a CD49b antigen. In some embodiments, the B cell antigen is a CD49c antigen. In some embodiments, the B cell antigen is a CD49d antigen. In some embodiments, the B cell antigen is a CD50 antigen. In some embodiments, the B cell antigen is a CD52 antigen. In some embodiments, the B cell antigen is a CD53 antigen. In some embodiments, the B cell antigen is a CD54 antigen. In some embodiments, the B cell antigen is a CD55 antigen. In some embodiments, the B cell antigen is a CD58 antigen. In some embodiments, the B cell antigen is a CD60a antigen. In some embodiments, the B cell antigen is a CD62L antigen. In some embodiments, the B cell antigen is a CD63 antigen. In some embodiments, the B cell antigen is a CD68 antigen. In some embodiments, the B cell antigen is a CD69 antigen. In some embodiments, the B cell antigen is a CD70 antigen. In some embodiments, the B cell antigen is a CD72 antigen. In some embodiments, the B cell antigen is a CD73 antigen. In some embodiments, the B cell antigen is a CD74 antigen. In some embodiments, the B cell antigen is a CD75 antigen. In some embodiments, the B cell antigen is a CD75S antigen. In some embodiments, the B cell antigen is a CD77 antigen. In some embodiments, the B cell antigen is a CD79a antigen. In some embodiments, the B cell antigen is a CD79b antigen. In some embodiments, the B cell antigen is a CD80 antigen. In some embodiments, the B cell antigen is a CD81 antigen. In some embodiments, the B cell antigen is a CD82 antigen. In some embodiments, the B cell antigen is a CD83 antigen. In some embodiments, the B cell antigen is a CD84 antigen. In some embodiments, the B cell antigen is a CD85E antigen. In some embodiments, the B cell antigen is a CD85I antigen. In some embodiments, the B cell antigen is a CD85J antigen. In some embodiments, the B cell antigen is a CD86 antigen. In some embodiments, the B cell antigen is a CD92 antigen. In some embodiments, the B cell antigen is a CD95 antigen. In some embodiments, the B cell antigen is a CD97 antigen. In some embodiments, the B cell antigen is a CD98 antigen. In some embodiments, the B cell antigen is a CD99 antigen. In some embodiments, the B cell antigen is a CD100 antigen. In some embodiments, the B cell antigen is a CD102 antigen. In some embodiments, the B cell antigen is a CD108 antigen. In some embodiments, the B cell antigen is a CD119 antigen. In some embodiments, the B cell antigen is a CD120a antigen. In some embodiments, the B cell antigen is a CD120b antigen. In some embodiments, the B cell antigen is a CD121b antigen. In some embodiments, the B cell antigen is a CD122 antigen. In some embodiments, the B cell antigen is a CD124 antigen. In some embodiments, the B cell antigen is a CD125 antigen. In some embodiments, the B cell antigen is a CD126 antigen. In some embodiments, the B cell antigen is a CD130 antigen. In some embodiments, the B cell antigen is a CD132 antigen. In some embodiments, the B cell antigen is a CD137 antigen. In some embodiments, the B cell antigen is a CD138 antigen. In some embodiments, the B cell antigen is a CD139 antigen. In some embodiments, the B cell antigen is a CD147 antigen. In some embodiments, the B cell antigen is a CD148 antigen. In some embodiments, the B cell antigen is a CD150 antigen. In some embodiments, the B cell antigen is a CD152 antigen. In some embodiments, the B cell antigen is a CD162 antigen. In some embodiments, the B cell antigen is a CD164 antigen. In some embodiments, the B cell antigen is a CD166 antigen. In some embodiments, the B cell antigen is a CD167a antigen. In some embodiments, the B cell antigen is a CD170 antigen. In some embodiments, the B cell antigen is a CD171 antigen. In some embodiments, the B cell antigen is a CD175 antigen. In some embodiments, the B cell antigen is a CD175s antigen. In some embodiments, the B cell antigen is a CD180 antigen. In some embodiments, the B cell antigen is a CD184 antigen. In some embodiments, the B cell antigen is a CD185 antigen. In some embodiments, the B cell antigen is a CD192 antigen. In some embodiments, the B cell antigen is a CD196 antigen. In some embodiments, the B cell antigen is a CD197 antigen. In some embodiments, the B cell antigen is a CD200 antigen. In some embodiments, the B cell antigen is a CD205 antigen. In some embodiments, the B cell antigen is a CD201a antigen. In some embodiments, the B cell antigen is a CDw210b antigen. In some embodiments, the B cell antigen is a CD212 antigen. In some embodiments, the B cell antigen is a CD213a1 antigen. In some embodiments, the B cell antigen is a CD213a2 antigen. In some embodiments, the B cell antigen is a CD 215 antigen. In some embodiments, the B cell antigen is a CD217 antigen. In some embodiments, the B cell antigen is a CD218a antigen. In some embodiments, the B cell antigen is a CD218b antigen. In some embodiments, the B cell antigen is a CD220 antigen. In some embodiments, the B cell antigen is a CD221 antigen. In some embodiments, the B cell antigen is a CD222 antigen. In some embodiments, the B cell antigen is a CD224 antigen. In some embodiments, the B cell antigen is a CD225 antigen. In some embodiments, the B cell antigen is a CD226 antigen. In some embodiments, the B cell antigen is a CD227 antigen. In some embodiments, the B cell antigen is a CD229 antigen. In some embodiments, the B cell antigen is a CD230 antigen. In some embodiments, the B cell antigen is a CD232 antigen. In some embodiments, the B cell antigen is a CD252 antigen. In some embodiments, the B cell antigen is a CD252 antigen. In some embodiments, the B cell antigen is a CD254 antigen. In some embodiments, the B cell antigen is a CD255 antigen. In some embodiments, the B cell antigen is a CD256 antigen. In some embodiments, the B cell antigen is a CD257 CD258 antigen. In some embodiments, the B cell antigen is a CD259 antigen. In some embodiments, the B cell antigen is a CD260 antigen. In some embodiments, the B cell antigen is a CD261 antigen. In some embodiments, the B cell antigen is a CD262 antigen. In some embodiments, the B cell antigen is a CD263 antigen. In some embodiments, the B cell antigen is a CD264 antigen. In some embodiments, the B cell antigen is a CD267-270 antigen. In some embodiments, the B cell antigen is a CD272 antigen. In some embodiments, the B cell antigen is a CD274 antigen. In some embodiments, the B cell antigen is a CD275 antigen. In some embodiments, the B cell antigen is a CD277 antigen. In some embodiments, the B cell antigen is a CD279 antigen. In some embodiments, the B cell antigen is a CD283 antigen. In some embodiments, the B cell antigen is a CD289 antigen. In some embodiments, the B cell antigen is a CD290 antigen. In some embodiments, the B cell antigen is a CD295 antigen. In some embodiments, the B cell antigen is a CD298 antigen. In some embodiments, the B cell antigen is a CD300 antigen. In some embodiments, the B cell antigen is a CD300c antigen. In some embodiments, the B cell antigen is a CD305 antigen. In some embodiments, the B cell antigen is a CD306 antigen. In some embodiments, the B cell antigen is a CD307a antigen. In some embodiments, the B cell antigen is a CD307b antigen. In some embodiments, the B cell antigen is a CD307c antigen. In some embodiments, the B cell antigen is a CD307d antigen. In some embodiments, the B cell antigen is a CD307e antigen. In some embodiments, the B cell antigen is a CD314 antigen. In some embodiments, the B cell antigen is a CD215 antigen. In some embodiments, the B cell antigen is a CD316 antigen. In some embodiments, the B cell antigen is a CD317 antigen. In some embodiments, the B cell antigen is a CD319 antigen. In some embodiments, the B cell antigen is a CD321 antigen. In some embodiments, the B cell antigen is a CD327 antigen. In some embodiments, the B cell antigen is a CD328 antigen. In some embodiments, the B cell antigen is a CD329 antigen. In some embodiments, the B cell antigen is a CD338 antigen. In some embodiments, the B cell antigen is a CD351 antigen. In some embodiments, the B cell antigen is a CD352 antigen. In some embodiments, the B cell antigen is a CD353 antigen. In some embodiments, the B cell antigen is a CD354 antigen. In some embodiments, the B cell antigen is a CD355 antigen. In some embodiments, the B cell antigen is a CD356 antigen. In some embodiments, the B cell antigen is a CD357 antigen. In some embodiments, the B cell antigen is a CD358 antigen. In some embodiments, the B cell antigen is a CD360 antigen. In some embodiments, the B cell antigen is a CD361 antigen. In some embodiments, the B cell antigen is a CD362 antigen. In some embodiments, the B cell antigen is a CD363 antigen.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is a dendritic cell. In a specific embodiment, the second target is a dendritic cell antigen. In some embodiments a multispecific TRGV9 antibody provided herein comprises: (a) a first binding domain that binds to TRGV9, and (b) a second binding domain that binds to a dendritic cell antigen present on the surface of a dendritic cell. In certain embodiments, the first binding domain of the multispecific TRGV9 antibody specifically binds TRGV9. In some embodiments, the TRGV9 is present on the surface of a γδ T cell. In some embodiments, the dendritic cell is killed when the multispecific antibody binds to the TRGV9 on the surface of the γδ T cell and the antigen on the surface of the dendritic cell. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of a dendritic cell. In some embodiments, provided herein is a bispecific antibody comprising: (a) a first binding domain that binds to a TRGV9 antigen, and (b) a second binding domain that binds to a dendritic cell antigen. In some embodiments, provided herein is a bispecific antibody comprising: (a) a first binding domain that specifically binds to a TRGV9 antigen, and (b) a second binding domain that specifically binds to a dendritic cell antigen. In some embodiments, provided herein is a bispecific antibody comprising: (a) a first binding domain that binds to a first epitope on a TRGV9 antigen, and (b) a second binding domain that binds to a second epitope on a dendritic cell antigen. In some embodiments, provided herein is a bispecific antibody comprising: (a) a first binding domain that specifically binds to a first epitope on a TRGV9 antigen, and (b) a second binding domain that specifically binds to a second epitope on a dendritic cell antigen. In an embodiment of the bispecific antibodies provided herein, the first epitope is located on TRGV9 and the second epitope is located on the surface of a dendritic cell. In some embodiments, the second epitope is located on a dendritic cell antigen.

In some embodiments, the dendritic cell antigen is a CD1a, CD1b, CD1c, CD1d, CD1e, CD11b, CD11c, CD16, CD17, CD18, CD19, CD21, CD23, CD29, CD33, CD35, CD36, CD38, CD39, CD40, CD45, CD45RA, CD45RB, CD45RC, CD45RO, CD48, CD49d, CD49e, CD58, CD64a, CD68, CD73, CD74, CD80, CD81, CD83, CD84, CD85A, CD85D, CD85E, CD85G, CD85J, CD86, CD88, CD97, CD101, CD116, CD120a, CD120b, CD123, CD139, CD148, CD150, CD156b, CD157, CD167, CD168, CD169, CD170, CD171, CD172a, CD172b, CD180, CD184, CD185, CD193, CD196, CD197, CD200, CD205, CD206, CD207, CD208, CD209, CDw210b, CD213a1, CD217, CD218a, CD218b, CD220, CD221, CD222, CD227, CD229, CD230, CD232, CD244, CD252, CD256, CD257, CD258, CD265, CD270, CD271, CD272, CD273, CD274, CD275, CD276, CD277, CD283, CD286, CD288, CD289, CD290, CD295, CD298, CD300a, CD300c, CD300e, CD301, CD302, CD303, CD304, CD305, CD312, CD317, CD319, CD320, CD328, CD352, CD354, CD357, or CD361 antigen. In some embodiments, the dendritic cell antigen is a CD1a antigen. In some embodiments, the dendritic cell antigen is a CD1b antigen. In some embodiments, the dendritic cell antigen is a CD1c antigen. In some embodiments, the dendritic cell antigen is a CD1d antigen. In some embodiments, the dendritic cell antigen is a CD1e antigen. In some embodiments, the dendritic cell antigen is a CD11b antigen. In some embodiments, the dendritic cell antigen is a CD11c antigen. In some embodiments, the dendritic cell antigen is a CD16 antigen. In some embodiments, the dendritic cell antigen is a CD17 antigen. In some embodiments, the dendritic cell antigen is a CD18 antigen. In some embodiments, the dendritic cell antigen is a CD19 antigen. In some embodiments, the dendritic cell antigen is a CD21 antigen. In some embodiments, the dendritic cell antigen is a CD23 antigen. In some embodiments, the dendritic cell antigen is a CD29 antigen. In some embodiments, the dendritic cell antigen is a CD33 antigen. In some embodiments, the dendritic cell antigen is a CD35 antigen. In some embodiments, the dendritic cell antigen is a CD36 antigen. In some embodiments, the dendritic cell antigen is a CD38 antigen. In some embodiments, the dendritic cell antigen is a CD39 antigen. In some embodiments, the dendritic cell antigen is a CD40 antigen. In some embodiments, the dendritic cell antigen is a CD45 antigen. In some embodiments, the dendritic cell antigen is a CD45RA antigen. In some embodiments, the dendritic cell antigen is a CD45RB antigen. In some embodiments, the dendritic cell antigen is a CD45RC antigen. In some embodiments, the dendritic cell antigen is a CD45RO antigen. In some embodiments, the dendritic cell antigen is a CD48 antigen. In some embodiments, the dendritic cell antigen is a CD49d antigen. In some embodiments, the dendritic cell antigen is a CD49e antigen. In some embodiments, the dendritic cell antigen is a CD58 antigen. In some embodiments, the dendritic cell antigen is a CD64a antigen. In some embodiments, the dendritic cell antigen is a CD68 antigen. In some embodiments, the dendritic cell antigen is a CD73 antigen. In some embodiments, the dendritic cell antigen is a CD74 antigen. In some embodiments, the dendritic cell antigen is a CD80 antigen. In some embodiments, the dendritic cell antigen is a CD81 antigen. In some embodiments, the dendritic cell antigen is a CD83 antigen. In some embodiments, the dendritic cell antigen is a CD84 antigen. In some embodiments, the dendritic cell antigen is a CD85A antigen. In some embodiments, the dendritic cell antigen is a CD85D antigen. In some embodiments, the dendritic cell antigen is a CD85E antigen. In some embodiments, the dendritic cell antigen is a CD85G antigen. In some embodiments, the dendritic cell antigen is a CD85J antigen. In some embodiments, the dendritic cell antigen is a CD86 antigen. In some embodiments, the dendritic cell antigen is a CD88 antigen. In some embodiments, the dendritic cell antigen is a CD97 antigen. In some embodiments, the dendritic cell antigen is a CD101 antigen. In some embodiments, the dendritic cell antigen is a CD116 antigen. In some embodiments, the dendritic cell antigen is a CD120a antigen. In some embodiments, the dendritic cell antigen is a CD120b antigen. In some embodiments, the dendritic cell antigen is a CD123 antigen. In some embodiments, the dendritic cell antigen is a CD139 antigen. In some embodiments, the dendritic cell antigen is a CD148 antigen. In some embodiments, the dendritic cell antigen is a CD150 antigen. In some embodiments, the dendritic cell antigen is a CD156b antigen. In some embodiments, the dendritic cell antigen is a CD157 antigen. In some embodiments, the dendritic cell antigen is a CD167 antigen. In some embodiments, the dendritic cell antigen is a CD168 antigen. In some embodiments, the dendritic cell antigen is a CD169 antigen. In some embodiments, the dendritic cell antigen is a CD170 antigen. In some embodiments, the dendritic cell antigen is a CD171 antigen. In some embodiments, the dendritic cell antigen is a CD172a antigen. In some embodiments, the dendritic cell antigen is a CD172b antigen. In some embodiments, the dendritic cell antigen is a CD180 antigen. In some embodiments, the dendritic cell antigen is a CD184 antigen. In some embodiments, the dendritic cell antigen is a CD185 antigen. In some embodiments, the dendritic cell antigen is a CD193 antigen. In some embodiments, the dendritic cell antigen is a CD196 antigen. In some embodiments, the dendritic cell antigen is a CD197 antigen. In some embodiments, the dendritic cell antigen is a CD200 antigen. In some embodiments, the dendritic cell antigen is a CD205 antigen. In some embodiments, the dendritic cell antigen is a CD206 antigen. In some embodiments, the dendritic cell antigen is a CD207 antigen. In some embodiments, the dendritic cell antigen is a CD208 antigen. In some embodiments, the dendritic cell antigen is a CD209 antigen. In some embodiments, the dendritic cell antigen is a CDw210b antigen. In some embodiments, the dendritic cell antigen is a CD213a1 antigen. In some embodiments, the dendritic cell antigen is a CD217 antigen. In some embodiments, the dendritic cell antigen is a CD218a antigen. In some embodiments, the dendritic cell antigen is a CD218b antigen. In some embodiments, the dendritic cell antigen is a CD220 antigen. In some embodiments, the dendritic cell antigen is a CD221 antigen. In some embodiments, the dendritic cell antigen is a CD222 antigen. In some embodiments, the dendritic cell antigen is a CD227 antigen. In some embodiments, the dendritic cell antigen is a CD229 antigen. In some embodiments, the dendritic cell antigen is a CD230 antigen. In some embodiments, the dendritic cell antigen is a CD232 antigen. In some embodiments, the dendritic cell antigen is a CD244 antigen. In some embodiments, the dendritic cell antigen is a CD252 antigen. In some embodiments, the dendritic cell antigen is a CD256 antigen. In some embodiments, the dendritic cell antigen is a CD257 antigen. In some embodiments, the dendritic cell antigen is a CD258 antigen. In some embodiments, the dendritic cell antigen is a CD265 antigen. In some embodiments, the dendritic cell antigen is a CD270 antigen. In some embodiments, the dendritic cell antigen is a CD271 antigen. In some embodiments, the dendritic cell antigen is a CD272 antigen. In some embodiments, the dendritic cell antigen is a CD273 antigen. In some embodiments, the dendritic cell antigen is a CD274 antigen. In some embodiments, the dendritic cell antigen is a CD275 antigen. In some embodiments, the dendritic cell antigen is a CD276 antigen. In some embodiments, the dendritic cell antigen is a CD277 antigen. In some embodiments, the dendritic cell antigen is a CD283 antigen. In some embodiments, the dendritic cell antigen is a CD286 antigen. In some embodiments, the dendritic cell antigen is a CD288 antigen. In some embodiments, the dendritic cell antigen is a CD289 antigen. In some embodiments, the dendritic cell antigen is a CD290 antigen. In some embodiments, the dendritic cell antigen is a CD295 antigen. In some embodiments, the dendritic cell antigen is a CD298 antigen. In some embodiments, the dendritic cell antigen is a CD300a antigen. In some embodiments, the dendritic cell antigen is a CD300c antigen. In some embodiments, the dendritic cell antigen is a CD300e antigen. In some embodiments, the dendritic cell antigen is a CD301 antigen. In some embodiments, the dendritic cell antigen is a CD302 antigen. In some embodiments, the dendritic cell antigen is a CD303 antigen. In some embodiments, the dendritic cell antigen is a CD304 antigen. In some embodiments, the dendritic cell antigen is a CD305 antigen. In some embodiments, the dendritic cell antigen is a CD312 antigen. In some embodiments, the dendritic cell antigen is a CD317 antigen. In some embodiments, the dendritic cell antigen is a CD319 antigen. In some embodiments, the dendritic cell antigen is a CD320 antigen. In some embodiments, the dendritic cell antigen is a CD328 antigen. In some embodiments, the dendritic cell antigen is a CD352 antigen. In some embodiments, the dendritic cell antigen is a CD354 antigen. In some embodiments, the dendritic cell antigen is a CD357 antigen. In some embodiments, the dendritic cell antigen is a CD361 antigen.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is a NK cell. In a specific embodiment, the second target is a NK cell antigen. In some embodiments a multispecific TRGV9 antibody provided herein comprises: (a) a first binding domain that binds to TRGV9, and (b) a second binding domain that binds to a NK cell antigen present on the surface of a NK cell. In certain embodiments, the first binding domain of the multispecific TRGV9 antibody specifically binds TRGV9. In some embodiments, the TRGV9 is present on the surface of a γδ T cell. In some embodiments, the NK cell is killed when the multispecific antibody binds to the TRGV9 on the surface of the γδ T cell and the antigen on the surface of the NK cell. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of a NK cell. In some embodiments, provided herein is a bispecific antibody comprising: (a) a first binding domain that binds to a TRGV9 antigen, and (b) a second binding domain that binds to a NK cell antigen. In some embodiments, provided herein is a bispecific antibody comprising: (a) a first binding domain that specifically binds to a TRGV9 antigen, and (b) a second binding domain that specifically binds to a NK cell antigen. In some embodiments, provided herein is a bispecific antibody comprising: (a) a first binding domain that binds to a first epitope on a TRGV9 antigen, and (b) a second binding domain that binds to a second epitope on a NK cell antigen. In some embodiments, provided herein is a bispecific antibody comprising: (a) a first binding domain that specifically binds to a first epitope on a TRGV9 antigen, and (b) a second binding domain that specifically binds to a second epitope on a NK cell antigen. In an embodiment of the bispecific antibodies provided herein, the first epitope is located on TRGV9 and the second epitope is located on the surface of a NK cell. In some embodiments, the second epitope is located on a NK cell antigen.

In some embodiments, the NK cell antigen is a CD2, CD7, CD8a, CD10, CD11a, CD11b, CD11c, CDw12, CD16, CD18, CD25, CD26, CD27, CD29, CD30, CD31, CD32c, CD38, CD39, CD43, CD44, CD45, CD45RA, CD45RB, CD45RC, CD45RO, CD46, CD47, CD48, CD49a, CD49b, CD49d, CD49e, CD50, CD52, CD53, CD55, CD56, CD7, CD58, CD59, CD62L, CD63, CD69, CD81, CD82, CD84, CD85C, CD85E, CD85J, CD87, CD94, CD95, CD96, CD97, CD98, CD99, CD99R, CD100, CD119, CD120a, CD120b, CD122, CD130, CD132, CD147, CD148, CD158a, CD158b1, CD158b2, CD158d, CD158e1/e2, CD158f, CD158g, CD158h, CD158i, CD158j, CD158k, CD159a, CD159c, CD160, CD161, CD172g, CD178, CD183, CD185, CDw210b, CD212, CD217, CD218a, CD218b, CD220, CD221, CD222, CD223, CD225, CD226, CD229, CD230, CD232, CD244, CD247, CD257, CD261, CD262, CD263, CD264, CD270, CD277, CD280, CD295, CD298, CD305, CD314, CD316, CD317, CD319, CD321, CD328, CD329, CD335, CD336, CD337, CD352, CD354, CD355, CD357, CD360, CD361, or CD363 antigen. In some embodiments, the NK cell antigen is a CD2 antigen. In some embodiments, the NK cell antigen is a CD7 antigen. In some embodiments, the NK cell antigen is a CD8a antigen. In some embodiments, the NK cell antigen is a CD10 antigen. In some embodiments, the NK cell antigen is a CD11a antigen. In some embodiments, the NK cell antigen is a CD11b antigen. In some embodiments, the NK cell antigen is a CD11c antigen. In some embodiments, the NK cell antigen is a CDw12 antigen. In some embodiments, the NK cell antigen is a CD16 antigen. In some embodiments, the NK cell antigen is a CD18 antigen. In some embodiments, the NK cell antigen is a CD25 antigen. In some embodiments, the NK cell antigen is a CD26 antigen. In some embodiments, the NK cell antigen is a CD27 antigen. In some embodiments, the NK cell antigen is a CD29 antigen. In some embodiments, the NK cell antigen is a CD30 antigen. In some embodiments, the NK cell antigen is a CD31 antigen. In some embodiments, the NK cell antigen is a CD32c antigen. In some embodiments, the NK cell antigen is a CD38 antigen. In some embodiments, the NK cell antigen is a CD39 antigen. In some embodiments, the NK cell antigen is a CD43 antigen. In some embodiments, the NK cell antigen is a CD44 antigen. In some embodiments, the NK cell antigen is a CD45 antigen. In some embodiments, the NK cell antigen is a CD45RA antigen. In some embodiments, the NK cell antigen is a CD45RB antigen. In some embodiments, the NK cell antigen is a CD45RC antigen. In some embodiments, the NK cell antigen is a CD45RO antigen. In some embodiments, the NK cell antigen is a CD46 antigen. In some embodiments, the NK cell antigen is a CD47 antigen. In some embodiments, the NK cell antigen is a CD48 antigen. In some embodiments, the NK cell antigen is a CD49a antigen. In some embodiments, the NK cell antigen is a CD49b antigen. In some embodiments, the NK cell antigen is a CD49d antigen. In some embodiments, the NK cell antigen is a CD49e antigen. In some embodiments, the NK cell antigen is a CD50 antigen. In some embodiments, the NK cell antigen is a CD52 antigen. In some embodiments, the NK cell antigen is a CD53 antigen. In some embodiments, the NK cell antigen is a CD55 antigen. In some embodiments, the NK cell antigen is a CD56 antigen. In some embodiments, the NK cell antigen is a CD7 antigen. In some embodiments, the NK cell antigen is a CD58 antigen. In some embodiments, the NK cell antigen is a CD59 antigen. In some embodiments, the NK cell antigen is a CD62L antigen. In some embodiments, the NK cell antigen is a CD63 antigen. In some embodiments, the NK cell antigen is a CD69 antigen. In some embodiments, the NK cell antigen is a CD81 antigen. In some embodiments, the NK cell antigen is a CD82 antigen. In some embodiments, the NK cell antigen is a CD84 antigen. In some embodiments, the NK cell antigen is a CD85C antigen. In some embodiments, the NK cell antigen is a CD85E antigen. In some embodiments, the NK cell antigen is a CD85J antigen. In some embodiments, the NK cell antigen is a CD87 antigen. In some embodiments, the NK cell antigen is a CD94 antigen. In some embodiments, the NK cell antigen is a CD95 antigen. In some embodiments, the NK cell antigen is a CD96 antigen. In some embodiments, the NK cell antigen is a CD97 antigen. In some embodiments, the NK cell antigen is a CD98 antigen. In some embodiments, the NK cell antigen is a CD99 antigen. In some embodiments, the NK cell antigen is a CD99R antigen. In some embodiments, the NK cell antigen is a CD100 antigen. In some embodiments, the NK cell antigen is a CD119 antigen. In some embodiments, the NK cell antigen is a CD120a antigen. In some embodiments, the NK cell antigen is a CD120b antigen. In some embodiments, the NK cell antigen is a CD122 antigen. In some embodiments, the NK cell antigen is a CD130 antigen. In some embodiments, the NK cell antigen is a CD132 antigen. In some embodiments, the NK cell antigen is a CD147 antigen. In some embodiments, the NK cell antigen is a CD148 antigen. In some embodiments, the NK cell antigen is a CD158a antigen. In some embodiments, the NK cell antigen is a CD158b1 antigen. In some embodiments, the NK cell antigen is a CD158b2 antigen. In some embodiments, the NK cell antigen is a CD158d antigen. In some embodiments, the NK cell antigen is a CD158e1/e2 antigen. In some embodiments, the NK cell antigen is a CD158f antigen. In some embodiments, the NK cell antigen is a CD158g antigen. In some embodiments, the NK cell antigen is a CD158h antigen. In some embodiments, the NK cell antigen is a CD158i antigen. In some embodiments, the NK cell antigen is a CD158j antigen. In some embodiments, the NK cell antigen is a CD158k antigen. In some embodiments, the NK cell antigen is a CD159a antigen. In some embodiments, the NK cell antigen is a CD159c antigen. In some embodiments, the NK cell antigen is a CD160 antigen. In some embodiments, the NK cell antigen is a CD161 antigen. In some embodiments, the NK cell antigen is a CD172g antigen. In some embodiments, the NK cell antigen is a CD178 antigen. In some embodiments, the NK cell antigen is a CD183 antigen. In some embodiments, the NK cell antigen is a CD185 antigen. In some embodiments, the NK cell antigen is a CDw210b antigen. In some embodiments, the NK cell antigen is a CD212 antigen. In some embodiments, the NK cell antigen is a CD217 antigen. In some embodiments, the NK cell antigen is a CD218a antigen. In some embodiments, the NK cell antigen is a CD218b antigen. In some embodiments, the NK cell antigen is a CD220 antigen. In some embodiments, the NK cell antigen is a CD221 antigen. In some embodiments, the NK cell antigen is a CD222 antigen. In some embodiments, the NK cell antigen is a CD223 antigen. In some embodiments, the NK cell antigen is a CD225 antigen. In some embodiments, the NK cell antigen is a CD226 antigen. In some embodiments, the NK cell antigen is a CD229 antigen. In some embodiments, the NK cell antigen is a CD230 antigen. In some embodiments, the NK cell antigen is a CD232 antigen. In some embodiments, the NK cell antigen is a CD244 antigen. In some embodiments, the NK cell antigen is a CD247 antigen. In some embodiments, the NK cell antigen is a CD257 antigen. In some embodiments, the NK cell antigen is a CD261 antigen. In some embodiments, the NK cell antigen is a CD262 antigen. In some embodiments, the NK cell antigen is a CD263 antigen. In some embodiments, the NK cell antigen is a CD264 antigen. In some embodiments, the NK cell antigen is a CD270 antigen. In some embodiments, the NK cell antigen is a CD277 antigen. In some embodiments, the NK cell antigen is a CD280 antigen. In some embodiments, the NK cell antigen is a CD295 antigen. In some embodiments, the NK cell antigen is a CD298 antigen. In some embodiments, the NK cell antigen is a CD305 antigen. In some embodiments, the NK cell antigen is a CD314 antigen. In some embodiments, the NK cell antigen is a CD316 antigen. In some embodiments, the NK cell antigen is a CD317 antigen. In some embodiments, the NK cell antigen is a CD319 antigen. In some embodiments, the NK cell antigen is a CD321 antigen. In some embodiments, the NK cell antigen is a CD328 antigen. In some embodiments, the NK cell antigen is a CD329 antigen. In some embodiments, the NK cell antigen is a CD335 antigen. In some embodiments, the NK cell antigen is a CD336 antigen. In some embodiments, the NK cell antigen is a CD337 antigen. In some embodiments, the NK cell antigen is a CD352 antigen. In some embodiments, the NK cell antigen is a CD354 antigen. In some embodiments, the NK cell antigen is a CD355 antigen. In some embodiments, the NK cell antigen is a CD357 antigen. In some embodiments, the NK cell antigen is a CD360 antigen. In some embodiments, the NK cell antigen is a CD361 antigen. In some embodiments, the NK cell antigen is a CD363 antigen.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is a stem cell. In a specific embodiment, the second target is a stem cell antigen. In certain embodiments, the target cell is a stem cell precursor. In a specific embodiment, the second target is a stem cell precursor antigen. In some embodiments a multispecific TRGV9 antibody provided herein comprises: (a) a first binding domain that binds to TRGV9, and (b) a second binding domain that binds to a stem cell or stem cell precursor antigen present on the surface of a stem cell or stem cell precursor. In certain embodiments, the first binding domain of the multispecific TRGV9 antibody specifically binds TRGV9. In some embodiments, the TRGV9 is present on the surface of a γδ T cell. In some embodiments, the stem cell or stem cell precursor is killed when the multispecific antibody binds to the TRGV9 on the surface of the γδ T cell and the antigen on the surface of the stem cell or stem cell precursor. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of a stem cell or stem cell precursor. In some embodiments, provided herein is a bispecific antibody comprising: (a) a first binding domain that binds to a TRGV9 antigen, and (b) a second binding domain that binds to a stem cell or stem cell precursor antigen. In some embodiments, provided herein is a bispecific antibody comprising: (a) a first binding domain that specifically binds to a TRGV9 antigen, and (b) a second binding domain that specifically binds to a stem cell or stem cell precursor antigen. In some embodiments, provided herein is a bispecific antibody comprising: (a) a first binding domain that binds to a first epitope on a TRGV9 antigen, and (b) a second binding domain that binds to a second epitope on a stem cell or stem cell precursor antigen. In some embodiments, provided herein is a bispecific antibody comprising: (a) a first binding domain that specifically binds to a first epitope on a TRGV9 antigen, and (b) a second binding domain that specifically binds to a second epitope on a stem cell or stem cell precursor antigen. In an embodiment of the bispecific antibodies provided herein, the first epitope is located on TRGV9 and the second epitope is located on the surface of a stem cell or stem cell precursor. In some embodiments, the second epitope is located on a stem cell or stem cell precursor antigen.

In some embodiments, the stem cell or stem cell precursor antigen is a CD8a, CDw12, CD13, CD15, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD135, CD138, CD151, CD157, CD162, CD164, CD168, CD172a, CD173, CD174, CD175, CD175s, CD176, CD183, CD191, CD200, CD201, CD205, CD217, CD220, CD221, CD222, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, or CD350 antigen. In some embodiments, the stem cell or stem cell precursor antigen is a CD8a antigen. In some embodiments, the stem cell or stem cell precursor antigen is a CDw12 antigen. In some embodiments, the stem cell or stem cell precursor antigen is a CD13 antigen. In some embodiments, the stem cell or stem cell precursor antigen is a CD15 antigen. In some embodiments, the stem cell or stem cell precursor antigen is a CD19 antigen. In some embodiments, the stem cell or stem cell precursor antigen is a CD21 antigen. In some embodiments, the stem cell or stem cell precursor antigen is a CD22 antigen. In some embodiments, the stem cell or stem cell precursor antigen is a CD29 antigen. In some embodiments, the stem cell or stem cell precursor antigen is a CD30 antigen. In some embodiments, the stem cell or stem cell precursor antigen is a CD33 antigen. In some embodiments, the stem cell or stem cell precursor antigen is a CD34 antigen. In some embodiments, the stem cell or stem cell precursor antigen is a CD36 antigen. In some embodiments, the stem cell or stem cell precursor antigen is a CD38 antigen. In some embodiments, the stem cell or stem cell precursor antigen is a CD40 antigen. In some embodiments, the stem cell or stem cell precursor antigen is a CD41 antigen. In some embodiments, the stem cell or stem cell precursor antigen is a CD42a antigen. In some embodiments, the stem cell or stem cell precursor antigen is a CD42b antigen. In some embodiments, the stem cell or stem cell precursor antigen is a CD42c antigen. In some embodiments, the stem cell or stem cell precursor antigen is a CD42d antigen. In some embodiments, the stem cell or stem cell precursor antigen is a CD43 antigen. In some embodiments, the stem cell or stem cell precursor antigen is a CD45 antigen. In some embodiments, the stem cell or stem cell precursor antigen is a CD45RA antigen. In some embodiments, the stem cell or stem cell precursor antigen is a CD45RB antigen. In some embodiments, the stem cell or stem cell precursor antigen is a CD45RC antigen. In some embodiments, the stem cell or stem cell precursor antigen is a CD45RO antigen. In some embodiments, the stem cell or stem cell precursor antigen is a CD48 antigen. In some embodiments, the stem cell or stem cell precursor antigen is a CD49b antigen. In some embodiments, the stem cell or stem cell precursor antigen is a CD49d antigen. In some embodiments, the stem cell or stem cell precursor antigen is a CD49e antigen. In some embodiments, the stem cell or stem cell precursor antigen is a CD49f antigen. In some embodiments, the stem cell or stem cell precursor antigen is a CD50 antigen. In some embodiments, the stem cell or stem cell precursor antigen is a CD53 antigen. In some embodiments, the stem cell or stem cell precursor antigen is a CD55 antigen. In some embodiments, the stem cell or stem cell precursor antigen is a CD64a antigen. In some embodiments, the stem cell or stem cell precursor antigen is a CD68 antigen. In some embodiments, the stem cell or stem cell precursor antigen is a CD71 antigen. In some embodiments, the stem cell or stem cell precursor antigen is a CD72 antigen. In some embodiments, the stem cell or stem cell precursor antigen is a CD73 antigen. In some embodiments, the stem cell or stem cell precursor antigen is a CD81 antigen. In some embodiments, the stem cell or stem cell precursor antigen is a CD82 antigen. In some embodiments, the stem cell or stem cell precursor antigen is a CD85A antigen. In some embodiments, the stem cell or stem cell precursor antigen is a CD85K antigen. In some embodiments, the stem cell or stem cell precursor antigen is a CD90 antigen. In some embodiments, the stem cell or stem cell precursor antigen is a CD99 antigen. In some embodiments, the stem cell or stem cell precursor antigen is a CD104 antigen. In some embodiments, the stem cell or stem cell precursor antigen is a CD105 antigen. In some embodiments, the stem cell or stem cell precursor antigen is a CD109 antigen. In some embodiments, the stem cell or stem cell precursor antigen is a CD110 antigen. In some embodiments, the stem cell or stem cell precursor antigen is a CD111 antigen. In some embodiments, the stem cell or stem cell precursor antigen is a CD112 antigen. In some embodiments, the stem cell or stem cell precursor antigen is a CD114 antigen. In some embodiments, the stem cell or stem cell precursor antigen is a CD115 antigen. In some embodiments, the stem cell or stem cell precursor antigen is a CD117 antigen. In some embodiments, the stem cell or stem cell precursor antigen is a CD123 antigen. In some embodiments, the stem cell or stem cell precursor antigen is a CD124 antigen. In some embodiments, the stem cell or stem cell precursor antigen is a CD126 antigen. In some embodiments, the stem cell or stem cell precursor antigen is a CD127 antigen. In some embodiments, the stem cell or stem cell precursor antigen is a CD130 antigen. In some embodiments, the stem cell or stem cell precursor antigen is a CD131 antigen. In some embodiments, the stem cell or stem cell precursor antigen is a CD133 antigen. In some embodiments, the stem cell or stem cell precursor antigen is a CD135 antigen. In some embodiments, the stem cell or stem cell precursor antigen is a CD138 antigen. In some embodiments, the stem cell or stem cell precursor antigen is a CD151 antigen. In some embodiments, the stem cell or stem cell precursor antigen is a CD157 antigen. In some embodiments, the stem cell or stem cell precursor antigen is a CD162 antigen. In some embodiments, the stem cell or stem cell precursor antigen is a CD164 antigen. In some embodiments, the stem cell or stem cell precursor antigen is a CD168 antigen. In some embodiments, the stem cell or stem cell precursor antigen is a CD172a antigen. In some embodiments, the stem cell or stem cell precursor antigen is a CD173 antigen. In some embodiments, the stem cell or stem cell precursor antigen is a CD174 antigen. In some embodiments, the stem cell or stem cell precursor antigen is a CD175 antigen. In some embodiments, the stem cell or stem cell precursor antigen is a CD175s antigen. In some embodiments, the stem cell or stem cell precursor antigen is a CD176 antigen. In some embodiments, the stem cell or stem cell precursor antigen is a CD183 antigen. In some embodiments, the stem cell or stem cell precursor antigen is a CD191 antigen. In some embodiments, the stem cell or stem cell precursor antigen is a CD200 antigen. In some embodiments, the stem cell or stem cell precursor antigen is a CD201 antigen. In some embodiments, the stem cell or stem cell precursor antigen is a CD205 antigen. In some embodiments, the stem cell or stem cell precursor antigen is a CD217 antigen. In some embodiments, the stem cell or stem cell precursor antigen is a CD220 antigen. In some embodiments, the stem cell or stem cell precursor antigen is a CD221 antigen. In some embodiments, the stem cell or stem cell precursor antigen is a CD222 antigen. In some embodiments, the stem cell or stem cell precursor antigen is a CD224 antigen. In some embodiments, the stem cell or stem cell precursor antigen is a CD225 antigen. In some embodiments, the stem cell or stem cell precursor antigen is a CD226 antigen. In some embodiments, the stem cell or stem cell precursor antigen is a CD227 antigen. In some embodiments, the stem cell or stem cell precursor antigen is a CD228 antigen. In some embodiments, the stem cell or stem cell precursor antigen is a CD229 antigen. In some embodiments, the stem cell or stem cell precursor antigen is a CD230 antigen. In some embodiments, the stem cell or stem cell precursor antigen is a CD235a antigen. In some embodiments, the stem cell or stem cell precursor antigen is a CD235b antigen. In some embodiments, the stem cell or stem cell precursor antigen is a CD236 antigen. In some embodiments, the stem cell or stem cell precursor antigen is a CD236R antigen. In some embodiments, the stem cell or stem cell precursor antigen is a CD238 antigen. In some embodiments, the stem cell or stem cell precursor antigen is a CD240 antigen. In some embodiments, the stem cell or stem cell precursor antigen is a CD242 antigen. In some embodiments, the stem cell or stem cell precursor antigen is a CD243 antigen. In some embodiments, the stem cell or stem cell precursor antigen is a CD277 antigen. In some embodiments, the stem cell or stem cell precursor antigen is a CD292 antigen. In some embodiments, the stem cell or stem cell precursor antigen is a CDw293 antigen. In some embodiments, the stem cell or stem cell precursor antigen is a CD295 antigen. In some embodiments, the stem cell or stem cell precursor antigen is a CD298 antigen. In some embodiments, the stem cell or stem cell precursor antigen is a CD309 antigen. In some embodiments, the stem cell or stem cell precursor antigen is a CD318 antigen. In some embodiments, the stem cell or stem cell precursor antigen is a CD324 antigen. In some embodiments, the stem cell or stem cell precursor antigen is a CD325 antigen. In some embodiments, the stem cell or stem cell precursor antigen is a CD338 antigen. In some embodiments, the stem cell or stem cell precursor antigen is a CD344 antigen. In some embodiments, the stem cell or stem cell precursor antigen is a CD349 antigen. In some embodiments, the stem cell or stem cell precursor antigen is a CD350 antigen.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is a monocyte. In a specific embodiment, the second target is a monocyte antigen. In certain embodiments, the target cell is a macrophage. In a specific embodiment, the second target is a macrophage antigen. In some embodiments a multispecific TRGV9 antibody provided herein comprises: (a) a first binding domain that binds to TRGV9, and (b) a second binding domain that binds to a macrophage or monocyte antigen present on the surface of a macrophage or monocyte. In certain embodiments, the first binding domain of the multispecific TRGV9 antibody specifically binds TRGV9. In some embodiments, the TRGV9 is present on the surface of a γδ T cell. In some embodiments, the macrophage or monocyte is killed when the multispecific antibody binds to the TRGV9 on the surface of the γδ T cell and the antigen on the surface of the macrophage or monocyte. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of a macrophage or monocyte. In some embodiments, provided herein is a bispecific antibody comprising: (a) a first binding domain that binds to a TRGV9 antigen, and (b) a second binding domain that binds to a macrophage or monocyte antigen. In some embodiments, provided herein is a bispecific antibody comprising: (a) a first binding domain that specifically binds to a TRGV9 antigen, and (b) a second binding domain that specifically binds to a macrophage or monocyte antigen. In some embodiments, provided herein is a bispecific antibody comprising: (a) a first binding domain that binds to a first epitope on a TRGV9 antigen, and (b) a second binding domain that binds to a second epitope on a macrophage or monocyte antigen. In some embodiments, provided herein is a bispecific antibody comprising: (a) a first binding domain that specifically binds to a first epitope on a TRGV9 antigen, and (b) a second binding domain that specifically binds to a second epitope on a macrophage or monocyte antigen. In an embodiment of the bispecific antibodies provided herein, the first epitope is located on TRGV9 and the second epitope is located on the surface of a macrophage or monocyte. In some embodiments, the second epitope is located on a macrophage or monocyte antigen.

In some embodiments, the macrophage or monocyte antigen is a CD1a, CD1b, CD1c, CD4, CD9, CD11a, CD11b, CD11c, CD11d, CDw12, CD13, CD14, CD15, CD16, CD17, CD18, CD23, CD25, CD26, CD29, CD30, CD31, CD32a, CD32b, CD32c, CD33, CD35, CD36, CD37, CD38, CD39, CD40, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD58, CD59, CD60a, CD61, CD63, CD64a, CD65, CD66, CD68, CD69, CD72, CD74, CD75, CD75S, CD80, CD81, CD82, CD84, CD85A, CD85C, CD85D, CD85E, CD85F, CD85G, CD85I, CD85J, CD85K, CD86, CD87, CD88, CD89, CD91, CD92, CD93, CD95, CD97, CD98, CD99, CD99R, CD100, CD101, CD102, CD105, CD111, CD112, CD114, CD115, CD116, CD119, CD120a, CD120b, CD121b, CD122, CD124, CD127, CD130, CD131, CD132, CD136, CD137, CD139, CD141, CD142, CD143, CD147, CD148, CD153, CD155, CD156a, CD156b, CD156c, CD157, CD162, CD163, CD164, CD165, CD166, CD168, CD169, CD170, CD171, CD172a, CD172b, CD180, CD181, CD182, CD184, CD185, CD191, CD192, CD194, CD195, CDw198, CD24, CD205, CD206, CD209, CD210a, CDw210b, CD213a1, CD213a2, CD217, CD220, CD221, CD222, CD224, CD226, CD227, CD230, CD232, CD244, CD252, CD256, CD257, CD258, CD261, CD262, CD263, CD264, CD265, CD267, CD268, CD270, CD272, CD273, CD274, CD275, CD276, CD277, CD280, CD281, CD282, CD284, CD286, CD288, CD289, CD295, CD297, CD298, CD300a, CD300c, CD300e, CD301, CD302, CD305, CD306, CD312, CD214, CD315, CD317, CD319, CD321, CD328, CD329, CD338, CD351, CD352, CD352, CD354, CD357, CD358, CD360, CD361, or CD362 antigen. In some embodiments, the macrophage or monocyte antigen is a CD1a antigen. In some embodiments, the macrophage or monocyte antigen is a CD1b antigen. In some embodiments, the macrophage or monocyte antigen is a CD1c antigen. In some embodiments, the macrophage or monocyte antigen is a CD4 antigen. In some embodiments, the macrophage or monocyte antigen is a CD9 antigen. In some embodiments, the macrophage or monocyte antigen is a CD11a antigen. In some embodiments, the macrophage or monocyte antigen is a CD11b antigen. In some embodiments, the macrophage or monocyte antigen is a CD11c antigen. In some embodiments, the macrophage or monocyte antigen is a CD11d antigen. In some embodiments, the macrophage or monocyte antigen is a CDw12 antigen. In some embodiments, the macrophage or monocyte antigen is a CD13 antigen. In some embodiments, the macrophage or monocyte antigen is a CD14 antigen. In some embodiments, the macrophage or monocyte antigen is a CD15 antigen. In some embodiments, the macrophage or monocyte antigen is a CD16 antigen. In some embodiments, the macrophage or monocyte antigen is a CD17 antigen. In some embodiments, the macrophage or monocyte antigen is a CD18 antigen. In some embodiments, the macrophage or monocyte antigen is a CD23 antigen. In some embodiments, the macrophage or monocyte antigen is a CD25 antigen. In some embodiments, the macrophage or monocyte antigen is a CD26 antigen. In some embodiments, the macrophage or monocyte antigen is a CD29 antigen. In some embodiments, the macrophage or monocyte antigen is a CD30 antigen. In some embodiments, the macrophage or monocyte antigen is a CD31 antigen. In some embodiments, the macrophage or monocyte antigen is a CD32a antigen. In some embodiments, the macrophage or monocyte antigen is a CD32b antigen. In some embodiments, the macrophage or monocyte antigen is a CD32c antigen. In some embodiments, the macrophage or monocyte antigen is a CD33 antigen. In some embodiments, the macrophage or monocyte antigen is a CD35 antigen. In some embodiments, the macrophage or monocyte antigen is a CD36 antigen. In some embodiments, the macrophage or monocyte antigen is a CD37 antigen. In some embodiments, the macrophage or monocyte antigen is a CD38 antigen. In some embodiments, the macrophage or monocyte antigen is a CD39 antigen. In some embodiments, the macrophage or monocyte antigen is a CD40 antigen. In some embodiments, the macrophage or monocyte antigen is a CD44 antigen. In some embodiments, the macrophage or monocyte antigen is a CD45 antigen. In some embodiments, the macrophage or monocyte antigen is a CD45RA antigen. In some embodiments, the macrophage or monocyte antigen is a CD45RB antigen. In some embodiments, the macrophage or monocyte antigen is a CD45RC antigen. In some embodiments, the macrophage or monocyte antigen is a CD45RO antigen. In some embodiments, the macrophage or monocyte antigen is a CD46 antigen. In some embodiments, the macrophage or monocyte antigen is a CD47 antigen. In some embodiments, the macrophage or monocyte antigen is a CD48 antigen. In some embodiments, the macrophage or monocyte antigen is a CD49a antigen. In some embodiments, the macrophage or monocyte antigen is a CD49b antigen. In some embodiments, the macrophage or monocyte antigen is a CD49c antigen. In some embodiments, the macrophage or monocyte antigen is a CD49d antigen. In some embodiments, the macrophage or monocyte antigen is a CD49e antigen. In some embodiments, the macrophage or monocyte antigen is a CD49f antigen. In some embodiments, the macrophage or monocyte antigen is a CD50 antigen. In some embodiments, the macrophage or monocyte antigen is a CD51 antigen. In some embodiments, the macrophage or monocyte antigen is a CD52 antigen. In some embodiments, the macrophage or monocyte antigen is a CD53 antigen. In some embodiments, the macrophage or monocyte antigen is a CD54 antigen. In some embodiments, the macrophage or monocyte antigen is a CD55 antigen. In some embodiments, the macrophage or monocyte antigen is a CD58 antigen. In some embodiments, the macrophage or monocyte antigen is a CD59 antigen. In some embodiments, the macrophage or monocyte antigen is a CD60a antigen. In some embodiments, the macrophage or monocyte antigen is a CD61 antigen. In some embodiments, the macrophage or monocyte antigen is a CD63 antigen. In some embodiments, the macrophage or monocyte antigen is a CD64a antigen. In some embodiments, the macrophage or monocyte antigen is a CD65 antigen. In some embodiments, the macrophage or monocyte antigen is a CD66 antigen. In some embodiments, the macrophage or monocyte antigen is a CD68 antigen. In some embodiments, the macrophage or monocyte antigen is a CD69 antigen. In some embodiments, the macrophage or monocyte antigen is a CD72 antigen. In some embodiments, the macrophage or monocyte antigen is a CD74 antigen. In some embodiments, the macrophage or monocyte antigen is a CD75 antigen. In some embodiments, the macrophage or monocyte antigen is a CD75S antigen. In some embodiments, the macrophage or monocyte antigen is a CD80 antigen. In some embodiments, the macrophage or monocyte antigen is a CD81 antigen. In some embodiments, the macrophage or monocyte antigen is a CD82 antigen. In some embodiments, the macrophage or monocyte antigen is a CD84 antigen. In some embodiments, the macrophage or monocyte antigen is a CD85A antigen. In some embodiments, the macrophage or monocyte antigen is a CD85C antigen. In some embodiments, the macrophage or monocyte antigen is a CD85D antigen. In some embodiments, the macrophage or monocyte antigen is a CD85E antigen. In some embodiments, the macrophage or monocyte antigen is a CD85F antigen. In some embodiments, the macrophage or monocyte antigen is a CD85G antigen. In some embodiments, the macrophage or monocyte antigen is a CD85I antigen. In some embodiments, the macrophage or monocyte antigen is a CD85J antigen. In some embodiments, the macrophage or monocyte antigen is a CD85K antigen. In some embodiments, the macrophage or monocyte antigen is a CD86 antigen. In some embodiments, the macrophage or monocyte antigen is a CD87 antigen. In some embodiments, the macrophage or monocyte antigen is a CD88 antigen. In some embodiments, the macrophage or monocyte antigen is a CD89 antigen. In some embodiments, the macrophage or monocyte antigen is a CD91 antigen. In some embodiments, the macrophage or monocyte antigen is a CD92 antigen. In some embodiments, the macrophage or monocyte antigen is a CD93 antigen. In some embodiments, the macrophage or monocyte antigen is a CD95 antigen. In some embodiments, the macrophage or monocyte antigen is a CD97 antigen. In some embodiments, the macrophage or monocyte antigen is a CD98 antigen. In some embodiments, the macrophage or monocyte antigen is a CD99 antigen. In some embodiments, the macrophage or monocyte antigen is a CD99R antigen. In some embodiments, the macrophage or monocyte antigen is a CD100 antigen. In some embodiments, the macrophage or monocyte antigen is a CD101 antigen. In some embodiments, the macrophage or monocyte antigen is a CD102 antigen. In some embodiments, the macrophage or monocyte antigen is a CD105 antigen. In some embodiments, the macrophage or monocyte antigen is a CD111 antigen. In some embodiments, the macrophage or monocyte antigen is a CD112 antigen. In some embodiments, the macrophage or monocyte antigen is a CD114 antigen. In some embodiments, the macrophage or monocyte antigen is a CD115 antigen. In some embodiments, the macrophage or monocyte antigen is a CD116 antigen. In some embodiments, the macrophage or monocyte antigen is a CD119 antigen. In some embodiments, the macrophage or monocyte antigen is a CD120a antigen. In some embodiments, the macrophage or monocyte antigen is a CD120b antigen. In some embodiments, the macrophage or monocyte antigen is a CD121b antigen. In some embodiments, the macrophage or monocyte antigen is a CD122 antigen. In some embodiments, the macrophage or monocyte antigen is a CD124 antigen. In some embodiments, the macrophage or monocyte antigen is a CD127 antigen. In some embodiments, the macrophage or monocyte antigen is a CD130 antigen. In some embodiments, the macrophage or monocyte antigen is a CD131 antigen. In some embodiments, the macrophage or monocyte antigen is a CD132 antigen. In some embodiments, the macrophage or monocyte antigen is a CD136 antigen. In some embodiments, the macrophage or monocyte antigen is a CD137 antigen. In some embodiments, the macrophage or monocyte antigen is a CD139 antigen. In some embodiments, the macrophage or monocyte antigen is a CD141 antigen. In some embodiments, the macrophage or monocyte antigen is a CD142 antigen. In some embodiments, the macrophage or monocyte antigen is a CD143 antigen. In some embodiments, the macrophage or monocyte antigen is a CD147 antigen. In some embodiments, the macrophage or monocyte antigen is a CD148 antigen. In some embodiments, the macrophage or monocyte antigen is a CD153 antigen. In some embodiments, the macrophage or monocyte antigen is a CD155 antigen. In some embodiments, the macrophage or monocyte antigen is a CD156a antigen. In some embodiments, the macrophage or monocyte antigen is a CD156b antigen. In some embodiments, the macrophage or monocyte antigen is a CD156c antigen. In some embodiments, the macrophage or monocyte antigen is a CD157 antigen. In some embodiments, the macrophage or monocyte antigen is a CD162 antigen. In some embodiments, the macrophage or monocyte antigen is a CD163 antigen. In some embodiments, the macrophage or monocyte antigen is a CD164 antigen. In some embodiments, the macrophage or monocyte antigen is a CD165 antigen. In some embodiments, the macrophage or monocyte antigen is a CD166 antigen. In some embodiments, the macrophage or monocyte antigen is a CD168 antigen. In some embodiments, the macrophage or monocyte antigen is a CD169 antigen. In some embodiments, the macrophage or monocyte antigen is a CD170 antigen. In some embodiments, the macrophage or monocyte antigen is a CD171 antigen. In some embodiments, the macrophage or monocyte antigen is a CD172a antigen. In some embodiments, the macrophage or monocyte antigen is a CD172b antigen. In some embodiments, the macrophage or monocyte antigen is a CD180 antigen. In some embodiments, the macrophage or monocyte antigen is a CD181 antigen. In some embodiments, the macrophage or monocyte antigen is a CD182 antigen. In some embodiments, the macrophage or monocyte antigen is a CD184 antigen. In some embodiments, the macrophage or monocyte antigen is a CD185 antigen. In some embodiments, the macrophage or monocyte antigen is a CD191 antigen. In some embodiments, the macrophage or monocyte antigen is a CD192 antigen. In some embodiments, the macrophage or monocyte antigen is a CD194 antigen. In some embodiments, the macrophage or monocyte antigen is a CD195 antigen. In some embodiments, the macrophage or monocyte antigen is a CDw198 antigen. In some embodiments, the macrophage or monocyte antigen is a CD24 antigen. In some embodiments, the macrophage or monocyte antigen is a CD205 antigen. In some embodiments, the macrophage or monocyte antigen is a CD206 antigen. In some embodiments, the macrophage or monocyte antigen is a CD209 antigen. In some embodiments, the macrophage or monocyte antigen is a CD210a antigen. In some embodiments, the macrophage or monocyte antigen is a CDw210b antigen. In some embodiments, the macrophage or monocyte antigen is a CD213a1 antigen. In some embodiments, the macrophage or monocyte antigen is a CD213a2 antigen. In some embodiments, the macrophage or monocyte antigen is a CD217 antigen. In some embodiments, the macrophage or monocyte antigen is a CD220 antigen. In some embodiments, the macrophage or monocyte antigen is a CD221 antigen. In some embodiments, the macrophage or monocyte antigen is a CD222 antigen. In some embodiments, the macrophage or monocyte antigen is a CD224 antigen. In some embodiments, the macrophage or monocyte antigen is a CD226 antigen. In some embodiments, the macrophage or monocyte antigen is a CD227 antigen. In some embodiments, the macrophage or monocyte antigen is a CD230 antigen. In some embodiments, the macrophage or monocyte antigen is a CD232 antigen. In some embodiments, the macrophage or monocyte antigen is a CD244 antigen. In some embodiments, the macrophage or monocyte antigen is a CD252 antigen. In some embodiments, the macrophage or monocyte antigen is a CD256 antigen. In some embodiments, the macrophage or monocyte antigen is a CD257 antigen. In some embodiments, the macrophage or monocyte antigen is a CD258 antigen. In some embodiments, the macrophage or monocyte antigen is a CD261 antigen. In some embodiments, the macrophage or monocyte antigen is a CD262 antigen. In some embodiments, the macrophage or monocyte antigen is a CD263 antigen. In some embodiments, the macrophage or monocyte antigen is a CD264 antigen. In some embodiments, the macrophage or monocyte antigen is a CD265 antigen. In some embodiments, the macrophage or monocyte antigen is a CD267 antigen. In some embodiments, the macrophage or monocyte antigen is a CD268 antigen. In some embodiments, the macrophage or monocyte antigen is a CD270 antigen. In some embodiments, the macrophage or monocyte antigen is a CD272 antigen. In some embodiments, the macrophage or monocyte antigen is a CD273 antigen. In some embodiments, the macrophage or monocyte antigen is a CD274 antigen. In some embodiments, the macrophage or monocyte antigen is a CD275 antigen. In some embodiments, the macrophage or monocyte antigen is a CD276 antigen. In some embodiments, the macrophage or monocyte antigen is a CD277 antigen. In some embodiments, the macrophage or monocyte antigen is a CD280 antigen. In some embodiments, the macrophage or monocyte antigen is a CD281 antigen. In some embodiments, the macrophage or monocyte antigen is a CD282 antigen. In some embodiments, the macrophage or monocyte antigen is a CD284 antigen. In some embodiments, the macrophage or monocyte antigen is a CD286 antigen. In some embodiments, the macrophage or monocyte antigen is a CD288 antigen. In some embodiments, the macrophage or monocyte antigen is a CD289 antigen. In some embodiments, the macrophage or monocyte antigen is a CD295 antigen. In some embodiments, the macrophage or monocyte antigen is a CD297 antigen. In some embodiments, the macrophage or monocyte antigen is a CD298 antigen. In some embodiments, the macrophage or monocyte antigen is a CD300a antigen. In some embodiments, the macrophage or monocyte antigen is a CD300c antigen. In some embodiments, the macrophage or monocyte antigen is a CD300e antigen. In some embodiments, the macrophage or monocyte antigen is a CD301 antigen. In some embodiments, the macrophage or monocyte antigen is a CD302 antigen. In some embodiments, the macrophage or monocyte antigen is a CD305 antigen. In some embodiments, the macrophage or monocyte antigen is a CD306 antigen. In some embodiments, the macrophage or monocyte antigen is a CD312 antigen. In some embodiments, the macrophage or monocyte antigen is a CD214 antigen. In some embodiments, the macrophage or monocyte antigen is a CD315 antigen. In some embodiments, the macrophage or monocyte antigen is a CD317 antigen. In some embodiments, the macrophage or monocyte antigen is a CD319 antigen. In some embodiments, the macrophage or monocyte antigen is a CD321 antigen. In some embodiments, the macrophage or monocyte antigen is a CD328 antigen. In some embodiments, the macrophage or monocyte antigen is a CD329 antigen. In some embodiments, the macrophage or monocyte antigen is a CD338 antigen. In some embodiments, the macrophage or monocyte antigen is a CD351 antigen. In some embodiments, the macrophage or monocyte antigen is a CD352 antigen. In some embodiments, the macrophage or monocyte antigen is a CD352 antigen. In some embodiments, the macrophage or monocyte antigen is a CD354 antigen. In some embodiments, the macrophage or monocyte antigen is a CD357 antigen. In some embodiments, the macrophage or monocyte antigen is a CD358 antigen. In some embodiments, the macrophage or monocyte antigen is a CD360 antigen. In some embodiments, the macrophage or monocyte antigen is a CD361 antigen. In some embodiments, the macrophage or monocyte antigen is a CD362 antigen.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is a granulocyte. In a specific embodiment, the second target is a granulocyte antigen. In some embodiments a multispecific TRGV9 antibody provided herein comprises: (a) a first binding domain that binds to TRGV9, and (b) a second binding domain that binds to a granulocyte antigen present on the surface of a granulocyte. In certain embodiments, the first binding domain of the multispecific TRGV9 antibody specifically binds TRGV9. In some embodiments, the TRGV9 is present on the surface of a γδ T cell. In some embodiments, the granulocyte is killed when the multispecific antibody binds to the TRGV9 on the surface of the γδ T cell and the antigen on the surface of the granulocyte. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of a granulocyte. In some embodiments, provided herein is a bispecific antibody comprising: (a) a first binding domain that binds to a TRGV9 antigen, and (b) a second binding domain that binds to a granulocyte antigen. In some embodiments, provided herein is a bispecific antibody comprising: (a) a first binding domain that specifically binds to a TRGV9 antigen, and (b) a second binding domain that specifically binds to a granulocyte antigen. In some embodiments, provided herein is a bispecific antibody comprising: (a) a first binding domain that binds to a first epitope on a TRGV9 antigen, and (b) a second binding domain that binds to a second epitope on a granulocyte antigen. In some embodiments, provided herein is a bispecific antibody comprising: (a) a first binding domain that specifically binds to a first epitope on a TRGV9 antigen, and (b) a second binding domain that specifically binds to a second epitope on a granulocyte antigen. In an embodiment of the bispecific antibodies provided herein, the first epitope is located on TRGV9 and the second epitope is located on the surface of a granulocyte. In some embodiments, the second epitope is located on a granulocyte antigen.

In some embodiments, the granulocyte antigen is a CD4, CD9, CD11a, CD11b, CD11c, CDw12, CD13, CD14, CD15, CD16, CD16b, CD17, CD18, CD23, CD24, CD29, CD31, CD32a, CD32b, CD32c, CD33, CD35, CD37, CD43, CD44, CD45, CD45RB, CD45RO, CD46, CD47, CD50, CD53, CD55, CD58, CD59, CD60a, CD62L, CD63, CD64a, CD65, CD65s, CD66a, CD66b, CD66c, CD66d, CD68, CD69, CD75S, CD82, CD85A, CD85D, CD85K, CD87, CD88, CD89, CD92, CD93, CD95, CD97, CD98, CD100, CD101, CD107a, CD107b, CD114, CD116, CD119, CD120a, CD120b, CD123, CD125, CD130, CD131, CD132, CD139, CD141, CD147, CD148, CD153, CD156a, CD156b, CD157, CD162, CD170, CD171, CD172a, CD177, CD178, CD181, CD182, CD183, CD192, CD193, CD195, CD203c, CD217, CD218a, CD218b, CD220, CD221, CD222, CD230, CD232, CD244, CD256, CD257, CD258, CD261, CD262, CD263, CD264, CD268, CD270, CD274, CD275, CD281, CD282, CD289, CD290, CD294, CD295, CD298, CD302, CD305, CD312, CD314, CD321, CD328, CD329, CD352, CD354, CD360, or CD362 antigen. In some embodiments, the granulocyte antigen is a CD4 antigen. In some embodiments, the granulocyte antigen is a CD9 antigen. In some embodiments, the granulocyte antigen is a CD11a antigen. In some embodiments, the granulocyte antigen is a CD11b antigen. In some embodiments, the granulocyte antigen is a CD11c antigen. In some embodiments, the granulocyte antigen is a CDw12 antigen. In some embodiments, the granulocyte antigen is a CD13 antigen. In some embodiments, the granulocyte antigen is a CD14 antigen. In some embodiments, the granulocyte antigen is a CD15 antigen. In some embodiments, the granulocyte antigen is a CD16 antigen. In some embodiments, the granulocyte antigen is a CD16b antigen. In some embodiments, the granulocyte antigen is a CD17 antigen. In some embodiments, the granulocyte antigen is a CD18 antigen. In some embodiments, the granulocyte antigen is a CD23 antigen. In some embodiments, the granulocyte antigen is a CD24 antigen. In some embodiments, the granulocyte antigen is a CD29 antigen. In some embodiments, the granulocyte antigen is a CD31 antigen. In some embodiments, the granulocyte antigen is a CD32a antigen. In some embodiments, the granulocyte antigen is a CD32b antigen. In some embodiments, the granulocyte antigen is a CD32c antigen. In some embodiments, the granulocyte antigen is a CD33 antigen. In some embodiments, the granulocyte antigen is a CD35 antigen. In some embodiments, the granulocyte antigen is a CD37 antigen. In some embodiments, the granulocyte antigen is a CD43 antigen. In some embodiments, the granulocyte antigen is a CD44 antigen. In some embodiments, the granulocyte antigen is a CD45 antigen. In some embodiments, the granulocyte antigen is a CD45RB antigen. In some embodiments, the granulocyte antigen is a CD45RO antigen. In some embodiments, the granulocyte antigen is a CD46 antigen. In some embodiments, the granulocyte antigen is a CD47 antigen. In some embodiments, the granulocyte antigen is a CD50 antigen. In some embodiments, the granulocyte antigen is a CD53 antigen. In some embodiments, the granulocyte antigen is a CD55 antigen. In some embodiments, the granulocyte antigen is a CD58 antigen. In some embodiments, the granulocyte antigen is a CD59 antigen. In some embodiments, the granulocyte antigen is a CD60a antigen. In some embodiments, the granulocyte antigen is a CD62L antigen. In some embodiments, the granulocyte antigen is a CD63 antigen. In some embodiments, the granulocyte antigen is a CD64a antigen. In some embodiments, the granulocyte antigen is a CD65 antigen. In some embodiments, the granulocyte antigen is a CD65s antigen. In some embodiments, the granulocyte antigen is a CD66a antigen. In some embodiments, the granulocyte antigen is a CD66b antigen. In some embodiments, the granulocyte antigen is a CD66c antigen. In some embodiments, the granulocyte antigen is a CD66d antigen. In some embodiments, the granulocyte antigen is a CD68 antigen. In some embodiments, the granulocyte antigen is a CD69 antigen. In some embodiments, the granulocyte antigen is a CD75S antigen. In some embodiments, the granulocyte antigen is a CD82 antigen. In some embodiments, the granulocyte antigen is a CD85A antigen. In some embodiments, the granulocyte antigen is a CD85D antigen. In some embodiments, the granulocyte antigen is a CD85K antigen. In some embodiments, the granulocyte antigen is a CD87 antigen. In some embodiments, the granulocyte antigen is a CD88 antigen. In some embodiments, the granulocyte antigen is a CD89 antigen. In some embodiments, the granulocyte antigen is a CD92 antigen. In some embodiments, the granulocyte antigen is a CD93 antigen. In some embodiments, the granulocyte antigen is a CD95 antigen. In some embodiments, the granulocyte antigen is a CD97 antigen. In some embodiments, the granulocyte antigen is a CD98 antigen. In some embodiments, the granulocyte antigen is a CD100 antigen. In some embodiments, the granulocyte antigen is a CD101 antigen. In some embodiments, the granulocyte antigen is a CD107a antigen. In some embodiments, the granulocyte antigen is a CD107b antigen. In some embodiments, the granulocyte antigen is a CD114 antigen. In some embodiments, the granulocyte antigen is a CD116 antigen. In some embodiments, the granulocyte antigen is a CD119 antigen. In some embodiments, the granulocyte antigen is a CD120a antigen. In some embodiments, the granulocyte antigen is a CD120b antigen. In some embodiments, the granulocyte antigen is a CD123 antigen. In some embodiments, the granulocyte antigen is a CD125 antigen. In some embodiments, the granulocyte antigen is a CD130 antigen. In some embodiments, the granulocyte antigen is a CD131 antigen. In some embodiments, the granulocyte antigen is a CD132 antigen. In some embodiments, the granulocyte antigen is a CD139 antigen. In some embodiments, the granulocyte antigen is a CD141 antigen. In some embodiments, the granulocyte antigen is a CD147 antigen. In some embodiments, the granulocyte antigen is a CD148 antigen. In some embodiments, the granulocyte antigen is a CD153 antigen. In some embodiments, the granulocyte antigen is a CD156a antigen. In some embodiments, the granulocyte antigen is a CD156b antigen. In some embodiments, the granulocyte antigen is a CD157 antigen. In some embodiments, the granulocyte antigen is a CD162 antigen. In some embodiments, the granulocyte antigen is a CD170 antigen. In some embodiments, the granulocyte antigen is a CD171 antigen. In some embodiments, the granulocyte antigen is a CD172a antigen. In some embodiments, the granulocyte antigen is a CD177 antigen. In some embodiments, the granulocyte antigen is a CD178 antigen. In some embodiments, the granulocyte antigen is a CD181 antigen. In some embodiments, the granulocyte antigen is a CD182 antigen. In some embodiments, the granulocyte antigen is a CD183 antigen. In some embodiments, the granulocyte antigen is a CD192 antigen. In some embodiments, the granulocyte antigen is a CD193 antigen. In some embodiments, the granulocyte antigen is a CD195 antigen. In some embodiments, the granulocyte antigen is a CD203c antigen. In some embodiments, the granulocyte antigen is a CD217 antigen. In some embodiments, the granulocyte antigen is a CD218a antigen. In some embodiments, the granulocyte antigen is a CD218b antigen. In some embodiments, the granulocyte antigen is a CD220 antigen. In some embodiments, the granulocyte antigen is a CD221 antigen. In some embodiments, the granulocyte antigen is a CD222 antigen. In some embodiments, the granulocyte antigen is a CD230 antigen. In some embodiments, the granulocyte antigen is a CD232 antigen. In some embodiments, the granulocyte antigen is a CD244 antigen. In some embodiments, the granulocyte antigen is a CD256 antigen. In some embodiments, the granulocyte antigen is a CD257 antigen. In some embodiments, the granulocyte antigen is a CD258 antigen. In some embodiments, the granulocyte antigen is a CD261 antigen. In some embodiments, the granulocyte antigen is a CD262 antigen. In some embodiments, the granulocyte antigen is a CD263 antigen. In some embodiments, the granulocyte antigen is a CD264 antigen. In some embodiments, the granulocyte antigen is a CD268 antigen. In some embodiments, the granulocyte antigen is a CD270 antigen. In some embodiments, the granulocyte antigen is a CD274 antigen. In some embodiments, the granulocyte antigen is a CD275 antigen. In some embodiments, the granulocyte antigen is a CD281 antigen. In some embodiments, the granulocyte antigen is a CD282 antigen. In some embodiments, the granulocyte antigen is a CD289 antigen. In some embodiments, the granulocyte antigen is a CD290 antigen. In some embodiments, the granulocyte antigen is a CD294 antigen. In some embodiments, the granulocyte antigen is a CD295 antigen. In some embodiments, the granulocyte antigen is a CD298 antigen. In some embodiments, the granulocyte antigen is a CD302 antigen. In some embodiments, the granulocyte antigen is a CD305 antigen. In some embodiments, the granulocyte antigen is a CD312 antigen. In some embodiments, the granulocyte antigen is a CD314 antigen. In some embodiments, the granulocyte antigen is a CD321 antigen. In some embodiments, the granulocyte antigen is a CD328 antigen. In some embodiments, the granulocyte antigen is a CD329 antigen. In some embodiments, the granulocyte antigen is a CD352 antigen. In some embodiments, the granulocyte antigen is a CD354 antigen. In some embodiments, the granulocyte antigen is a CD360 antigen. In some embodiments, the granulocyte antigen is a CD362 antigen.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is a platelet. In a specific embodiment, the second target is a platelet antigen. In some embodiments a multispecific TRGV9 antibody provided herein comprises: (a) a first binding domain that binds to TRGV9, and (b) a second binding domain that binds to a platelet antigen present on the surface of a platelet. In certain embodiments, the first binding domain of the multispecific TRGV9 antibody specifically binds TRGV9. In some embodiments, the TRGV9 is present on the surface of a γδ T cell. In some embodiments, the platelet is killed when the multispecific antibody binds to the TRGV9 on the surface of the γδ T cell and the antigen on the surface of the platelet. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of a platelet. In some embodiments, provided herein is a bispecific antibody comprising: (a) a first binding domain that binds to a TRGV9 antigen, and (b) a second binding domain that binds to a platelet antigen. In some embodiments, provided herein is a bispecific antibody comprising: (a) a first binding domain that specifically binds to a TRGV9 antigen, and (b) a second binding domain that specifically binds to a platelet antigen. In some embodiments, provided herein is a bispecific antibody comprising: (a) a first binding domain that binds to a first epitope on a TRGV9 antigen, and (b) a second binding domain that binds to a second epitope on a platelet antigen. In some embodiments, provided herein is a bispecific antibody comprising: (a) a first binding domain that specifically binds to a first epitope on a TRGV9 antigen, and (b) a second binding domain that specifically binds to a second epitope on a platelet antigen. In an embodiment of the bispecific antibodies provided herein, the first epitope is located on TRGV9 and the second epitope is located on the surface of a platelet. In some embodiments, the second epitope is located on a platelet antigen.

In some embodiments, the platelet antigen is a CD9, CD17, CD18, CD23, CD29, CD31, CD32a, CD32b, CD36, CD41, CD42a, CD42b, CD42c, CD42d, CD43, CD46, CD47, CD62P, CD63, CD69, CD82, CD84, CD98, CD99, CD107a, CD107b, CD109, CD110, CD111, CD112, CD114, CD140a, CD141, CD147, CD148, CD151, CD165, CD194, CD226, CD295, CD298, or CD321 antigen. In some embodiments, the platelet antigen is a CD9 antigen. In some embodiments, the platelet antigen is a CD17 antigen. In some embodiments, the platelet antigen is a CD18 antigen. In some embodiments, the platelet antigen is a CD23 antigen. In some embodiments, the platelet antigen is a CD29 antigen. In some embodiments, the platelet antigen is a CD31 antigen. In some embodiments, the platelet antigen is a CD32a antigen. In some embodiments, the platelet antigen is a CD32b antigen. In some embodiments, the platelet antigen is a CD36 antigen. In some embodiments, the platelet antigen is a CD41 antigen. In some embodiments, the platelet antigen is a CD42a antigen. In some embodiments, the platelet antigen is a CD42b antigen. In some embodiments, the platelet antigen is a CD42c antigen. In some embodiments, the platelet antigen is a CD42d antigen. In some embodiments, the platelet antigen is a CD43 antigen. In some embodiments, the platelet antigen is a CD46 antigen. In some embodiments, the platelet antigen is a CD47 antigen. In some embodiments, the platelet antigen is a CD62P antigen. In some embodiments, the platelet antigen is a CD63 antigen. In some embodiments, the platelet antigen is a CD69 antigen. In some embodiments, the platelet antigen is a CD82 antigen. In some embodiments, the platelet antigen is a CD84 antigen. In some embodiments, the platelet antigen is a CD98 antigen. In some embodiments, the platelet antigen is a CD99 antigen. In some embodiments, the platelet antigen is a CD107a antigen. In some embodiments, the platelet antigen is a CD107b antigen. In some embodiments, the platelet antigen is a CD109 antigen. In some embodiments, the platelet antigen is a CD110 antigen. In some embodiments, the platelet antigen is a CD111 antigen. In some embodiments, the platelet antigen is a CD112 antigen. In some embodiments, the platelet antigen is a CD114 antigen. In some embodiments, the platelet antigen is a CD140a antigen. In some embodiments, the platelet antigen is a CD141 antigen. In some embodiments, the platelet antigen is a CD147 antigen. In some embodiments, the platelet antigen is a CD148 antigen. In some embodiments, the platelet antigen is a CD151 antigen. In some embodiments, the platelet antigen is a CD165 antigen. In some embodiments, the platelet antigen is a CD194 antigen. In some embodiments, the platelet antigen is a CD226 antigen. In some embodiments, the platelet antigen is a CD295 antigen. In some embodiments, the platelet antigen is a CD298 antigen. In some embodiments, the platelet antigen is a CD321 antigen.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is an erythrocyte. In a specific embodiment, the second target is an erythrocyte antigen. In some embodiments a multispecific TRGV9 antibody provided herein comprises: (a) a first binding domain that binds to TRGV9, and (b) a second binding domain that binds to an erythrocyte antigen present on the surface of an erythrocyte. In certain embodiments, the first binding domain of the multispecific TRGV9 antibody specifically binds TRGV9. In some embodiments, the TRGV9 is present on the surface of a γδ T cell. In some embodiments, the erythrocyte is killed when the multispecific antibody binds to the TRGV9 on the surface of the γδ T cell and the antigen on the surface of the erythrocyte. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of an erythrocyte. In some embodiments, provided herein is a bispecific antibody comprising: (a) a first binding domain that binds to a TRGV9 antigen, and (b) a second binding domain that binds to an erythrocyte antigen. In some embodiments, provided herein is a bispecific antibody comprising: (a) a first binding domain that specifically binds to a TRGV9 antigen, and (b) a second binding domain that specifically binds to an erythrocyte antigen. In some embodiments, provided herein is a bispecific antibody comprising: (a) a first binding domain that binds to a first epitope on a TRGV9 antigen, and (b) a second binding domain that binds to a second epitope on an erythrocyte antigen. In some embodiments, provided herein is a bispecific antibody comprising: (a) a first binding domain that specifically binds to a first epitope on a TRGV9 antigen, and (b) a second binding domain that specifically binds to a second epitope on an erythrocyte antigen. In an embodiment of the bispecific antibodies provided herein, the first epitope is located on TRGV9 and the second epitope is located on the surface of an erythrocyte. In some embodiments, the second epitope is located on an erythrocyte antigen.

In some embodiments, the erythrocyte antigen is a CD35, CD36, CD44, CD47, CD49e, CD55, CD58, CD59, CD75S, CD99, CD108, CD111, CD139, CD147, CD173, CD176, CD233, CD234, CD235a, CD235b, CD236, CD236R, CD238, CD239, CD240, CD241, CD242, or CD324 antigen. In some embodiments, the erythrocyte antigen is a CD35 antigen. In some embodiments, the erythrocyte antigen is a CD36 antigen. In some embodiments, the erythrocyte antigen is a CD44 antigen. In some embodiments, the erythrocyte antigen is a CD47 antigen. In some embodiments, the erythrocyte antigen is a CD49e antigen. In some embodiments, the erythrocyte antigen is a CD55 antigen. In some embodiments, the erythrocyte antigen is a CD58 antigen. In some embodiments, the erythrocyte antigen is a CD59 antigen. In some embodiments, the erythrocyte antigen is a CD75S antigen. In some embodiments, the erythrocyte antigen is a CD99 antigen. In some embodiments, the erythrocyte antigen is a CD108 antigen. In some embodiments, the erythrocyte antigen is a CD111 antigen. In some embodiments, the erythrocyte antigen is a CD139 antigen. In some embodiments, the erythrocyte antigen is a CD147 antigen. In some embodiments, the erythrocyte antigen is a CD173 antigen. In some embodiments, the erythrocyte antigen is a CD176 antigen. In some embodiments, the erythrocyte antigen is a CD233 antigen. In some embodiments, the erythrocyte antigen is a CD234 antigen. In some embodiments, the erythrocyte antigen is a CD235a antigen. In some embodiments, the erythrocyte antigen is a CD235b antigen. In some embodiments, the erythrocyte antigen is a CD236 antigen. In some embodiments, the erythrocyte antigen is a CD236R antigen. In some embodiments, the erythrocyte antigen is a CD238 antigen. In some embodiments, the erythrocyte antigen is a CD239 antigen. In some embodiments, the erythrocyte antigen is a CD240 antigen. In some embodiments, the erythrocyte antigen is a CD241 antigen. In some embodiments, the erythrocyte antigen is a CD242 antigen. In some embodiments, the erythrocyte antigen is a CD324 antigen.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is an endothelial cell. In a specific embodiment, the second target is an endothelial cell antigen. In some embodiments a multispecific TRGV9 antibody provided herein comprises: (a) a first binding domain that binds to TRGV9, and (b) a second binding domain that binds to an endothelial cell antigen present on the surface of an endothelial cell. In certain embodiments, the first binding domain of the multispecific TRGV9 antibody specifically binds TRGV9. In some embodiments, the TRGV9 is present on the surface of a γδ T cell. In some embodiments, the endothelial cell is killed when the multispecific antibody binds to the TRGV9 on the surface of the γδ T cell and the antigen on the surface of the endothelial cell. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of an endothelial cell. In some embodiments, provided herein is a bispecific antibody comprising: (a) a first binding domain that binds to a TRGV9 antigen, and (b) a second binding domain that binds to an endothelial cell antigen. In some embodiments, provided herein is a bispecific antibody comprising: (a) a first binding domain that specifically binds to a TRGV9 antigen, and (b) a second binding domain that specifically binds to an endothelial cell antigen. In some embodiments, provided herein is a bispecific antibody comprising: (a) a first binding domain that binds to a first epitope on a TRGV9 antigen, and (b) a second binding domain that binds to a second epitope on an endothelial cell antigen. In some embodiments, provided herein is a bispecific antibody comprising: (a) a first binding domain that specifically binds to a first epitope on a TRGV9 antigen, and (b) a second binding domain that specifically binds to a second epitope on an endothelial cell antigen. In an embodiment of the bispecific antibodies provided herein, the first epitope is located on TRGV9 and the second epitope is located on the surface of an endothelial cell. In some embodiments, the second epitope is located on an endothelial cell antigen.

In some embodiments, the endothelial cell antigen is a CD9, CD10, CD13, CD17, CD29, CD30, CD31, CD32b, CD34, CD36, CD39, CD40, CD44, CD46, CD47, CD49b, CD49c, CD49d, CD4e, CD49f, CD50, CD51, CD54, CD5, CD58, CD61, CD62E, CD62P, CD63, CD71, CD73, CD74, CD75S, CD77, CD81, CD82, CD86, CD87, CD88, CD90, CD92, CD93, CD98, CD99, CD102, CD104, CD105, CD106, CD107a, CD107b, CD109, CD110, CD111, CD112, CD114, CD117, CD119, CD120a, CD120b, CD121a, CD123, CD130, CD133, CD138, CD140a, CD140b, CD141, CD142, CD143, CD144, CDw154, CD146, CD147, CD150, CD151, CD156b, CD157, CD166, CD171, CD173, CD175S, CD176, CD178, CD184, CD192, CD200, CD201, CD202b, CD206, CD209, CD213a1, CD217, CD218a, CD220, CD221, CD222, CD224, CD225, CD228, CD230, CD234, CD239, CD242, CD246, CD248, CD252, CD266, CD280, 295, CD297, CD299, CD309, CD321, CD322, or CD344 antigen. In some embodiments, the endothelial cell antigen is a CD9 antigen. In some embodiments, the endothelial cell antigen is a CD10 antigen. In some embodiments, the endothelial cell antigen is a CD13 antigen. In some embodiments, the endothelial cell antigen is a CD17 antigen. In some embodiments, the endothelial cell antigen is a CD29 antigen. In some embodiments, the endothelial cell antigen is a CD30 antigen. In some embodiments, the endothelial cell antigen is a CD31 antigen. In some embodiments, the endothelial cell antigen is a CD32b antigen. In some embodiments, the endothelial cell antigen is a CD34 antigen. In some embodiments, the endothelial cell antigen is a CD36 antigen. In some embodiments, the endothelial cell antigen is a CD39 antigen. In some embodiments, the endothelial cell antigen is a CD40 antigen. In some embodiments, the endothelial cell antigen is a CD44 antigen. In some embodiments, the endothelial cell antigen is a CD46 antigen. In some embodiments, the endothelial cell antigen is a CD47 antigen. In some embodiments, the endothelial cell antigen is a CD49b antigen. In some embodiments, the endothelial cell antigen is a CD49c antigen. In some embodiments, the endothelial cell antigen is a CD49d antigen. In some embodiments, the endothelial cell antigen is a CD4e antigen. In some embodiments, the endothelial cell antigen is a CD49f antigen. In some embodiments, the endothelial cell antigen is a CD50 antigen. In some embodiments, the endothelial cell antigen is a CD51 antigen. In some embodiments, the endothelial cell antigen is a CD54 antigen. In some embodiments, the endothelial cell antigen is a CD5 antigen. In some embodiments, the endothelial cell antigen is a CD58 antigen. In some embodiments, the endothelial cell antigen is a CD61 antigen. In some embodiments, the endothelial cell antigen is a CD62E antigen. In some embodiments, the endothelial cell antigen is a CD62P antigen. In some embodiments, the endothelial cell antigen is a CD63 antigen. In some embodiments, the endothelial cell antigen is a CD71 antigen. In some embodiments, the endothelial cell antigen is a CD73 antigen. In some embodiments, the endothelial cell antigen is a CD74 antigen. In some embodiments, the endothelial cell antigen is a CD75S antigen. In some embodiments, the endothelial cell antigen is a CD77 antigen. In some embodiments, the endothelial cell antigen is a CD81 antigen. In some embodiments, the endothelial cell antigen is a CD82 antigen. In some embodiments, the endothelial cell antigen is a CD86 antigen. In some embodiments, the endothelial cell antigen is a CD87 antigen. In some embodiments, the endothelial cell antigen is a CD88 antigen. In some embodiments, the endothelial cell antigen is a CD90 antigen. In some embodiments, the endothelial cell antigen is a CD92 antigen. In some embodiments, the endothelial cell antigen is a CD93 antigen. In some embodiments, the endothelial cell antigen is a CD98 antigen. In some embodiments, the endothelial cell antigen is a CD99 antigen. In some embodiments, the endothelial cell antigen is a CD102 antigen. In some embodiments, the endothelial cell antigen is a CD104 antigen. In some embodiments, the endothelial cell antigen is a CD105 antigen. In some embodiments, the endothelial cell antigen is a CD106 antigen. In some embodiments, the endothelial cell antigen is a CD107a antigen. In some embodiments, the endothelial cell antigen is a CD107b antigen. In some embodiments, the endothelial cell antigen is a CD109 antigen. In some embodiments, the endothelial cell antigen is a CD110 antigen. In some embodiments, the endothelial cell antigen is a CD111 antigen. In some embodiments, the endothelial cell antigen is a CD112 antigen. In some embodiments, the endothelial cell antigen is a CD114 antigen. In some embodiments, the endothelial cell antigen is a CD117 antigen. In some embodiments, the endothelial cell antigen is a CD119 antigen. In some embodiments, the endothelial cell antigen is a CD120a antigen. In some embodiments, the endothelial cell antigen is a CD120b antigen. In some embodiments, the endothelial cell antigen is a CD121a antigen. In some embodiments, the endothelial cell antigen is a CD123 antigen. In some embodiments, the endothelial cell antigen is a CD130 antigen. In some embodiments, the endothelial cell antigen is a CD133 antigen. In some embodiments, the endothelial cell antigen is a CD138. In some embodiments, the endothelial cell antigen is a CD140a antigen. In some embodiments, the endothelial cell antigen is a CD140b antigen. In some embodiments, the endothelial cell antigen is a CD141 antigen. In some embodiments, the endothelial cell antigen is a CD142 antigen. In some embodiments, the endothelial cell antigen is a CD143 antigen. In some embodiments, the endothelial cell antigen is a CD144 antigen. In some embodiments, the endothelial cell antigen is a CDw154 antigen. In some embodiments, the endothelial cell antigen is a CD146 antigen. In some embodiments, the endothelial cell antigen is a CD147 antigen. In some embodiments, the endothelial cell antigen is a CD150 antigen. In some embodiments, the endothelial cell antigen is a CD151 antigen. In some embodiments, the endothelial cell antigen is a CD156b antigen. In some embodiments, the endothelial cell antigen is a CD157 antigen. In some embodiments, the endothelial cell antigen is a CD166 antigen. In some embodiments, the endothelial cell antigen is a CD171 antigen. In some embodiments, the endothelial cell antigen is a CD173 antigen. In some embodiments, the endothelial cell antigen is a CD175S antigen. In some embodiments, the endothelial cell antigen is a CD176 antigen. In some embodiments, the endothelial cell antigen is a CD178 antigen. In some embodiments, the endothelial cell antigen is a CD184 antigen. In some embodiments, the endothelial cell antigen is a CD192 antigen. In some embodiments, the endothelial cell antigen is a CD200 antigen. In some embodiments, the endothelial cell antigen is a CD201 antigen. In some embodiments, the endothelial cell antigen is a CD202b antigen. In some embodiments, the endothelial cell antigen is a CD206 antigen. In some embodiments, the endothelial cell antigen is a CD209 antigen. In some embodiments, the endothelial cell antigen is a CD213a1 antigen. In some embodiments, the endothelial cell antigen is a CD217 antigen. In some embodiments, the endothelial cell antigen is a CD218a antigen. In some embodiments, the endothelial cell antigen is a CD220 antigen. In some embodiments, the endothelial cell antigen is a CD221 antigen. In some embodiments, the endothelial cell antigen is a CD222 antigen. In some embodiments, the endothelial cell antigen is a CD224 antigen. In some embodiments, the endothelial cell antigen is a CD225 antigen. In some embodiments, the endothelial cell antigen is a CD228 antigen. In some embodiments, the endothelial cell antigen is a CD230 antigen. In some embodiments, the endothelial cell antigen is a CD234 antigen. In some embodiments, the endothelial cell antigen is a CD239 antigen. In some embodiments, the endothelial cell antigen is a CD242 antigen. In some embodiments, the endothelial cell antigen is a CD246 antigen. In some embodiments, the endothelial cell antigen is a CD248 antigen. In some embodiments, the endothelial cell antigen is a CD252 antigen. In some embodiments, the endothelial cell antigen is a CD266 antigen. In some embodiments, the endothelial cell antigen is a CD280, 295 antigen. In some embodiments, the endothelial cell antigen is a CD297 antigen. In some embodiments, the endothelial cell antigen is a CD299 antigen. In some embodiments, the endothelial cell antigen is a CD309 antigen. In some embodiments, the endothelial cell antigen is a CD321 antigen. In some embodiments, the endothelial cell antigen is a CD322 antigen. In some embodiments, the endothelial cell antigen is a CD344 antigen.

In one embodiment of the multispecific TRGV9 antibodies provided herein, the second binding arm binds a second target. In one embodiment, the second target is present on a target cell. In one embodiment, the second target is present on the surface of a target cell. In certain embodiments, the target cell is an epithelial cell. In a specific embodiment, the second target is an epithelial cell antigen. In some embodiments a multispecific TRGV9 antibody provided herein comprises: (a) a first binding domain that binds to TRGV9, and (b) a second binding domain that binds to an epithelial cell antigen present on the surface of an epithelial cell. In certain embodiments, the first binding domain of the multispecific TRGV9 antibody specifically binds TRGV9. In some embodiments, the TRGV9 is present on the surface of a γδ T cell. In some embodiments, the epithelial cell is killed when the multispecific antibody binds to the TRGV9 on the surface of the γδ T cell and the antigen on the surface of the epithelial cell. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of an epithelial cell. In some embodiments, provided herein is a bispecific antibody comprising: (a) a first binding domain that binds to a TRGV9 antigen, and (b) a second binding domain that binds to an epithelial cell antigen. In some embodiments, provided herein is a bispecific antibody comprising: (a) a first binding domain that specifically binds to a TRGV9 antigen, and (b) a second binding domain that specifically binds to an epithelial cell antigen. In some embodiments, provided herein is a bispecific antibody comprising: (a) a first binding domain that binds to a first epitope on a TRGV9 antigen, and (b) a second binding domain that binds to a second epitope on an epithelial cell antigen. In some embodiments, provided herein is a bispecific antibody comprising: (a) a first binding domain that specifically binds to a first epitope on a TRGV9 antigen, and (b) a second binding domain that specifically binds to a second epitope on an epithelial cell antigen. In an embodiment of the bispecific antibodies provided herein, the first epitope is located on TRGV9 and the second epitope is located on the surface of an epithelial cell. In some embodiments, the second epitope is located on an epithelial cell antigen.

In some embodiments, the epithelial cell antigen is a CD1d, CD9, CD13, CD18, CD21, CD23, CD24, CD26, CD29, CD39, CD40, CD44, CD46, CD47, CD49b, CD49c, CD49e, CD49f, CD52, CD55, CD58, CD66a, CD66c, CD66e, CD66f, CD73, CD74, CD75S, CD77, CD81, CD82, CD88, 91, CD92, CD98, CD99, CD104, CD110, CD111, CD112, CD113, CD114, CD118, CD120a, CD120b, CD124, CD129, CD133, CD136, CD137, CD138, CD141, CD142, CD143, CDw145, CD151, CD164, CD165, CD166, CD167a, CD171, CD174, CD175, CD175S, CD176, CD178, CD193, CD206, CD213a2, CD217, CD220, CD221, CD222, CD224, CD227, CD230, CD234, CD239, CD249, CD286, CD295, CD296, CD321, CD324, CD326, CD331, CD332, CD333, CD334, CD339, CD340, CD344, or CD350 antigen. In some embodiments, the epithelial cell antigen is a CD1d antigen. In some embodiments, the epithelial cell antigen is a CD9 antigen. In some embodiments, the epithelial cell antigen is a CD13 antigen. In some embodiments, the epithelial cell antigen is a CD18 antigen. In some embodiments, the epithelial cell antigen is a CD21 antigen. In some embodiments, the epithelial cell antigen is a CD23 antigen. In some embodiments, the epithelial cell antigen is a CD24 antigen. In some embodiments, the epithelial cell antigen is a CD26 antigen. In some embodiments, the epithelial cell antigen is a CD29 antigen. In some embodiments, the epithelial cell antigen is a CD39 antigen. In some embodiments, the epithelial cell antigen is a CD40 antigen. In some embodiments, the epithelial cell antigen is a CD44 antigen. In some embodiments, the epithelial cell antigen is a CD46 antigen. In some embodiments, the epithelial cell antigen is a CD47 antigen. In some embodiments, the epithelial cell antigen is a CD49b antigen. In some embodiments, the epithelial cell antigen is a CD49c antigen. In some embodiments, the epithelial cell antigen is a CD49e antigen. In some embodiments, the epithelial cell antigen is a CD49f antigen. In some embodiments, the epithelial cell antigen is a CD52 antigen. In some embodiments, the epithelial cell antigen is a CD55 antigen. In some embodiments, the epithelial cell antigen is a CD58 antigen. In some embodiments, the epithelial cell antigen is a CD66a antigen. In some embodiments, the epithelial cell antigen is a CD66c antigen. In some embodiments, the epithelial cell antigen is a CD66e antigen. In some embodiments, the epithelial cell antigen is a CD66f antigen. In some embodiments, the epithelial cell antigen is a CD73 antigen. In some embodiments, the epithelial cell antigen is a CD74 antigen. In some embodiments, the epithelial cell antigen is a CD75S antigen. In some embodiments, the epithelial cell antigen is a CD77 antigen. In some embodiments, the epithelial cell antigen is a CD81 antigen. In some embodiments, the epithelial cell antigen is a CD82 antigen. In some embodiments, the epithelial cell antigen is a CD88, 91 antigen. In some embodiments, the epithelial cell antigen is a CD92 antigen. In some embodiments, the epithelial cell antigen is a CD98 antigen. In some embodiments, the epithelial cell antigen is a CD99 antigen. In some embodiments, the epithelial cell antigen is a CD104 antigen. In some embodiments, the epithelial cell antigen is a CD110 antigen. In some embodiments, the epithelial cell antigen is a CD111 antigen. In some embodiments, the epithelial cell antigen is a CD112 antigen. In some embodiments, the epithelial cell antigen is a CD113 antigen. In some embodiments, the epithelial cell antigen is a CD114 antigen. In some embodiments, the epithelial cell antigen is a CD118 antigen. In some embodiments, the epithelial cell antigen is a CD120a antigen. In some embodiments, the epithelial cell antigen is a CD120b antigen. In some embodiments, the epithelial cell antigen is a CD124 antigen. In some embodiments, the epithelial cell antigen is a CD129 antigen. In some embodiments, the epithelial cell antigen is a CD133 antigen. In some embodiments, the epithelial cell antigen is a CD136 antigen. In some embodiments, the epithelial cell antigen is a CD137 antigen. In some embodiments, the epithelial cell antigen is a CD138 antigen. In some embodiments, the epithelial cell antigen is a CD141 antigen. In some embodiments, the epithelial cell antigen is a CD142 antigen. In some embodiments, the epithelial cell antigen is a CD143 antigen. In some embodiments, the epithelial cell antigen is a CDw145 antigen. In some embodiments, the epithelial cell antigen is a CD151 antigen. In some embodiments, the epithelial cell antigen is a CD164 antigen. In some embodiments, the epithelial cell antigen is a CD165 antigen. In some embodiments, the epithelial cell antigen is a CD166 antigen. In some embodiments, the epithelial cell antigen is a CD167a antigen. In some embodiments, the epithelial cell antigen is a CD171 antigen. In some embodiments, the epithelial cell antigen is a CD174 antigen. In some embodiments, the epithelial cell antigen is a CD175 antigen. In some embodiments, the epithelial cell antigen is a CD175S antigen. In some embodiments, the epithelial cell antigen is a CD176 antigen. In some embodiments, the epithelial cell antigen is a CD178 antigen. In some embodiments, the epithelial cell antigen is a CD193 antigen. In some embodiments, the epithelial cell antigen is a CD206 antigen. In some embodiments, the epithelial cell antigen is a CD213a2 antigen. In some embodiments, the epithelial cell antigen is a CD217 antigen. In some embodiments, the epithelial cell antigen is a CD220 antigen. In some embodiments, the epithelial cell antigen is a CD221 antigen. In some embodiments, the epithelial cell antigen is a CD222 antigen. In some embodiments, the epithelial cell antigen is a CD224 antigen. In some embodiments, the epithelial cell antigen is a CD227 antigen. In some embodiments, the epithelial cell antigen is a CD230 antigen. In some embodiments, the epithelial cell antigen is a CD234 antigen. In some embodiments, the epithelial cell antigen is a CD239 antigen. In some embodiments, the epithelial cell antigen is a CD249 antigen. In some embodiments, the epithelial cell antigen is a CD286 antigen. In some embodiments, the epithelial cell antigen is a CD295 antigen. In some embodiments, the epithelial cell antigen is a CD296 antigen. In some embodiments, the epithelial cell antigen is a CD321 antigen. In some embodiments, the epithelial cell antigen is a CD324 antigen. In some embodiments, the epithelial cell antigen is a CD326 antigen. In some embodiments, the epithelial cell antigen is a CD331 antigen. In some embodiments, the epithelial cell antigen is a CD332 antigen. In some embodiments, the epithelial cell antigen is a CD333 antigen. In some embodiments, the epithelial cell antigen is a CD334 antigen. In some embodiments, the epithelial cell antigen is a CD339 antigen. In some embodiments, the epithelial cell antigen is a CD340 antigen. In some embodiments, the epithelial cell antigen is a CD344 antigen. In some embodiments, the epithelial cell antigen is a CD350 antigen.

In one embodiment of the multispecific TRGV9 antibodies provided herein, the second binding arm binds a second target. In one embodiment, the second target is a pathogen. In one embodiment, the second target is present on a target cell. In one embodiment, the second target is present on the surface of a target cell. In certain embodiments, the target cell is a cell comprising a pathogen. In a specific embodiment, the second target is a pathogen antigen. In some embodiments a multispecific TRGV9 antibody provided herein comprises: (a) a first binding domain that binds to TRGV9, and (b) a second binding domain that binds to a pathogen antigen present on the surface of a cell comprising a pathogen. In certain embodiments, the first binding domain of the multispecific TRGV9 antibody specifically binds TRGV9. In some embodiments, the TRGV9 is present on the surface of a γδ T cell. In some embodiments, the cell comprising the pathogen is killed when the multispecific antibody binds to the TRGV9 on the surface of the γδ T cell and the antigen on the surface of the cell. In some embodiments, the multispecific TRGV9 antibody is a bispecific TRGV9 antibody. Bispecific antibodies comprising any of the TRGV9 antibodies provided herein as the first binding domain are contemplated. In certain embodiments, the TRGV9 antibody binds to a first epitope located on TRGV9 and a second epitope of a pathogen. In some embodiments, provided herein is a bispecific antibody comprising: (a) a first binding domain that binds to a TRGV9 antigen, and (b) a second binding domain that binds to a pathogen antigen. In some embodiments, provided herein is a bispecific antibody comprising: (a) a first binding domain that specifically binds to a TRGV9 antigen, and (b) a second binding domain that specifically binds to a pathogen antigen. In some embodiments, provided herein is a bispecific antibody comprising: (a) a first binding domain that binds to a first epitope on a TRGV9 antigen, and (b) a second binding domain that binds to a second epitope on a pathogen antigen. In some embodiments, provided herein is a bispecific antibody comprising: (a) a first binding domain that specifically binds to a first epitope on a TRGV9 antigen, and (b) a second binding domain that specifically binds to a second epitope on a pathogen antigen. In an embodiment of the bispecific antibodies provided herein, the first epitope is located on TRGV9 and the second epitope is located on the surface of a cell comprising a pathogen. In some embodiments, the second epitope is located on a pathogen antigen.

In some embodiments, the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru(EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection. In some embodiments, the infectious disease is Acute Flaccid Myelitis (AFM). In some embodiments, the infectious disease is Anaplasmosis. In some embodiments, the infectious disease is Anthrax. In some embodiments, the infectious disease is Babesiosis. In some embodiments, the infectious disease is Botulism. In some embodiments, the infectious disease is Brucellosis. In some embodiments, the infectious disease is Campylobacteriosis. In some embodiments, the infectious disease is Carbapenem-resistant Infection. In some embodiments, the infectious disease is Chancroid. In some embodiments, the infectious disease is Chikungunya Virus Infection. In some embodiments, the infectious disease is Chlamydia. In some embodiments, the infectious disease is Ciguatera. In some embodiments, the infectious disease is Difficile Infection. In some embodiments, the infectious disease is Perfringens. In some embodiments, the infectious disease is Coccidioidomycosis fungal infection. In some embodiments, the infectious disease is coronavirus. In some embodiments, the infectious disease is Covid-19 (SARS-CoV-2). In some embodiments, the infectious disease is Creutzfeldt-Jacob Disease/transmissible spongiform encephalopathy. In some embodiments, the infectious disease is Cryptosporidiosis (Crypto). In some embodiments, the infectious disease is Cyclosporiasis. In some embodiments, the infectious disease is Dengue 1, 2, 3 or 4. In some embodiments, the infectious disease is Diphtheria. In some embodiments, the infectious disease is E. coli infection/Shiga toxin-producing (STEC). In some embodiments, the infectious disease is Eastern Equine Encephalitis. In some embodiments, the infectious disease is Hemorrhagic Fever (Ebola). In some embodiments, the infectious disease is Ehrlichiosis. In some embodiments, the infectious disease is Encephalitis. In some embodiments, the infectious disease is Arboviral or parainfectious. In some embodiments, the infectious disease is Non-Polio Enterovirus. In some embodiments, the infectious disease is D68 Enteroviru(EV-D68). In some embodiments, the infectious disease is Giardiasis. In some embodiments, the infectious disease is Glanders. In some embodiments, the infectious disease is Gonococcal Infection. In some embodiments, the infectious disease is Granuloma inguinale. In some embodiments, the infectious disease is Haemophilus Influenza disease Type B (Hib or H-flu). In some embodiments, the infectious disease is Hantavirus Pulmonary Syndrome (HPS). In some embodiments, the infectious disease is Hemolytic Uremic Syndrome (HUS). In some embodiments, the infectious disease is Hepatitis A (Hep A). In some embodiments, the infectious disease is Hepatitis B (Hep B). In some embodiments, the infectious disease is Hepatitis C (Hep C). In some embodiments, the infectious disease is Hepatitis D (Hep D). In some embodiments, the infectious disease is Hepatitis E (Hep E). In some embodiments, the infectious disease is Herpes. In some embodiments, the infectious disease is Herpes Zoster (Shingles). In some embodiments, the infectious disease is Histoplasmosis infection. In some embodiments, the infectious disease is Human Immunodeficiency Virus/AIDS (HIV/AIDS). In some embodiments, the infectious disease is Human Papillomavirus (HPV). In some embodiments, the infectious disease is Influenza (Flu). In some embodiments, the infectious disease is Legionellosis (Legionnaires Disease). In some embodiments, the infectious disease is Leprosy (Hansens Disease). In some embodiments, the infectious disease is Leptospirosis. In some embodiments, the infectious disease is Listeriosis (Listeria). In some embodiments, the infectious disease is Lyme Disease. In some embodiments, the infectious disease is Lymphogranuloma venereum infection (LGV). In some embodiments, the infectious disease is Malaria. In some embodiments, the infectious disease is Measles. In some embodiments, the infectious disease is Melioidosis. In some embodiments, the infectious disease is Meningitis (Viral). In some embodiments, the infectious disease is Meningococcal Disease (Meningitis (Bacterial)). In some embodiments, the infectious disease is Middle East Respiratory Syndrome Coronavirus (MERS-CoV). In some embodiments, the infectious disease is Mumps. In some embodiments, the infectious disease is Norovirus. In some embodiments, the infectious disease is Pediculosis. In some embodiments, the infectious disease is Pelvic Inflammatory Disease (PID). In some embodiments, the infectious disease is Pertussis (Whooping Cough). In some embodiments, the infectious disease is Plague (Bubonic. In some embodiments, the infectious disease is Septicemic. In some embodiments, the infectious disease is Pneumonic). In some embodiments, the infectious disease is Pneumococcal Disease (Pneumonia). In some embodiments, the infectious disease is Poliomyelitis (Polio). In some embodiments, the infectious disease is Powassan. In some embodiments, the infectious disease is Psittacosis. In some embodiments, the infectious disease is Pthiriasis. In some embodiments, the infectious disease is Pustular Rash diseases (Small pox. In some embodiments, the infectious disease is monkeypox. In some embodiments, the infectious disease is cowpox). In some embodiments, the infectious disease is Q-Fever. In some embodiments, the infectious disease is Rabies. In some embodiments, the infectious disease is Rickettsiosis (Rocky Mountain Spotted Fever). In some embodiments, the infectious disease is Rubella (German Measles). In some embodiments, the infectious disease is Salmonellosis gastroenteritis (Salmonella). In some embodiments, the infectious disease is Scabies. In some embodiments, the infectious disease is Scombroid. In some embodiments, the infectious disease is Sepsis. In some embodiments, the infectious disease is Severe Acute Respiratory Syndrome (SARS). In some embodiments, the infectious disease is Shigellosis gastroenteritis (Shigella). In some embodiments, the infectious disease is Smallpox. In some embodiments, the infectious disease is Staphyloccal Infection Methicillin-resistant (MRSA). In some embodiments, the infectious disease is Staphylococcal Food Poisoning Enterotoxin B Poisoning (Staph Food Poisoning). In some embodiments, the infectious disease is Saphylococcal Infection Vancomycin Intermediate (VISA). In some embodiments, the infectious disease is Staphylococcal Infection Vancomycin Resistant (VRSA). In some embodiments, the infectious disease is Streptococcal Disease Group A (invasive) (Strep A (invasive). In some embodiments, the infectious disease is Streptococcal Disease. In some embodiments, the infectious disease is Group B (Strep-B). In some embodiments, the infectious disease is Streptococcal Toxic-Shock Syndrome STSS Toxic Shock. In some embodiments, the infectious disease is Syphilis (primary. In some embodiments, the infectious disease is secondary. In some embodiments, the infectious disease is early latent. In some embodiments, the infectious disease is late latent. In some embodiments, the infectious disease is congenital). In some embodiments, the infectious disease is Tetanus Infection. In some embodiments, the infectious disease is Trichomoniasis. In some embodiments, the infectious disease is Trichonosis Infection. In some embodiments, the infectious disease is Tuberculosis (TB). In some embodiments, the infectious disease is Tuberculosis Latent (LTBI). In some embodiments, the infectious disease is Tularemia. In some embodiments, the infectious disease is Typhoid Fever Group D. In some embodiments, the infectious disease is Vaginosis. In some embodiments, the infectious disease is Varicella (Chickenpox), Vibrio cholerae (Cholera). In some embodiments, the infectious disease is Vibriosis (Vibrio). In some embodiments, the infectious disease is Ebola Virus Hemorrhagic Fever. In some embodiments, the infectious disease is Lasa Virus Hemorrhagic Fever. In some embodiments, the infectious disease is Marburg Virus Hemorrhagic Fever. In some embodiments, the infectious disease is West Nile Virus. In some embodiments, the infectious disease is Yellow Fever. In some embodiments, the infectious disease is Yersenia. In some embodiments, the infectious disease is and Zika Virus Infection.

In some embodiments, the pathogen is a virus. In some embodiments, the virus is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, paramyxoviridae, parvoviridae, picornaviridae, polyomaviridae, poxviridae, reoviridae, retroviridae, rhabdoviridae, or togaviridae family. In some embodiments family. In some embodiments, the virus is a virus of the virus is a virus of the adenoviridae family. In some embodiments, the virus is a virus of the arenaviridae family. In some embodiments, the virus is a virus of the astroviridae family. In some embodiments, the virus is a virus of the bunyaviridae family. In some embodiments, the virus is a virus of the caliciviridae family. In some embodiments, the virus is a virus of the coronaviridae family. In some embodiments, the virus is a virus of the filoviridae family. In some embodiments, the virus is a virus of the flaviviridae family. In some embodiments, the virus is a virus of the hepadnaviridae family. In some embodiments, the virus is a virus of the hepeviridae family. In some embodiments, the virus is a virus of the orthomyxoviridae family. In some embodiments, the virus is a virus of the papillomaviridae family. In some embodiments, the virus is a virus of the paramyxoviridae family. In some embodiments, the virus is a virus of the parvoviridae family. In some embodiments, the virus is a virus of the picornaviridae family. In some embodiments, the virus is a virus of the polyomaviridae family. In some embodiments, the virus is a virus of the poxviridae family. In some embodiments, the virus is a virus of the reoviridae family. In some embodiments, the virus is a virus of the retroviridae family. In some embodiments, the virus is a virus of the rhabdoviridae family. In some embodiments, the virus is a virus of the togaviridae family.

In some embodiments, the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus. In some embodiments, the virus is an adenovirus. In some embodiments, the virus is a coronavirus. In some embodiments, the coronavirus virus is Covid-19 (SARS-CoV-2). In some embodiments, the virus is a coxsackievirus. In some embodiments, the virus is a Epstein-Barr virus. In some embodiments, the virus is a hepatitis A virus. In some embodiments, the virus is a hepatitis B virus. In some embodiments, the virus is a hepatitis C virus. In some embodiments, the virus is a herpes simplex virus type 2. In some embodiments, the virus is a cytomegalovirus. In some embodiments, the virus is a human herpes virus type 8. In some embodiments, the virus is a human immunodeficiency virus. In some embodiments, the virus is an influenza virus. In some embodiments, the virus is a measles virus. In some embodiments, the virus is a mumps virus. In some embodiments, the virus is a human papillomavirus. In some embodiments, the virus is a parainfluenza virus. In some embodiments, the virus is a poliovirus. In some embodiments, the virus is a rabies virus. In some embodiments, the virus is a respiratory syncytial virus. In some embodiments, the virus is a rubella virus. In some embodiments, the virus is a varicella-zoster virus.

In some embodiments, the pathogen is a bacteria. In some embodiments, the bacteria is a bacteria of a bacillus, bartonella, bordetella, borrelia, brucella, campylobacter, chlamydia, chlamydophila, clostridium, corynebacterium, enterococcus, escherichia, francisella, haemophilus, helicobacter, legionella, leptospira, listeria, mycobacterium, mycoplasma, neisseria, pseudomonas, rickettsia, salmonella, shigella, staphylococcus, streptococcus, treponema, ureaplasma, vibrio or yersinia genus. In some embodiments, the bacteria is a bacteria of the bacillus genus. In some embodiments, the bacteria is a bacteria of the bartonella genus. In some embodiments, the bacteria is a bacteria of the bordetella genus. In some embodiments, the bacteria is a bacteria of the borrelia genus. In some embodiments, the bacteria is a bacteria of the brucella genus. In some embodiments, the bacteria is a bacteria of the campylobacter genus. In some embodiments, the bacteria is a bacteria of the chlamydia genus. In some embodiments, the bacteria is a bacteria of the chlamydophila genus. In some embodiments, the bacteria is a bacteria of the clostridium genus. In some embodiments, the bacteria is a bacteria of the corynebacterium genus. In some embodiments, the bacteria is a bacteria of the enterococcus genus. In some embodiments, the bacteria is a bacteria of the escherichia genus. In some embodiments, the bacteria is a bacteria of the francisella genus. In some embodiments, the bacteria is a bacteria of the haemophilus genus. In some embodiments, the bacteria is a bacteria of the helicobacter genus. In some embodiments, the bacteria is a bacteria of the legionella genus. In some embodiments, the bacteria is a bacteria of the leptospira genus. In some embodiments, the bacteria is a bacteria of the listeria genus. In some embodiments, the bacteria is a bacteria of the mycobacterium genus. In some embodiments, the bacteria is a bacteria of the mycoplasma genus. In some embodiments, the bacteria is a bacteria of the neisseria genus. In some embodiments, the bacteria is a bacteria of the pseudomonas genus. In some embodiments, the bacteria is a bacteria of the rickettsia genus. In some embodiments, the bacteria is a bacteria of the salmonella genus. In some embodiments, the bacteria is a bacteria of the shigella genus. In some embodiments, the bacteria is a bacteria of the staphylococcus genus. In some embodiments, the bacteria is a bacteria of the streptococcus genus. In some embodiments, the bacteria is a bacteria of the treponema genus. In some embodiments, the bacteria is a bacteria of the ureaplasma genus. In some embodiments, the bacteria is a bacteria of the vibrio genus. In some embodiments, the bacteria is a bacteria of the yersinia genus.

In some embodiments, the pathogen is a parasite. In some embodiments, the parasite is a protozoa, helminth, or ectoparasite. In some embodiments, the protozoa is an entamoeba, giardia, leishmania, balantidium, plasmodium, or cryptosporidium. In some embodiments, the helminth is a trematode, cestode, acanthocephalan, or round worm. In some embodiments, the ectoparasite is a arthropod.

In some embodiments, the first binding domain that binds to TRGV9 is chimeric. In some embodiments, the first binding domain that binds to TRGV9 is human. In some embodiments, the first binding domain that binds to TRGV9 is humanized. In certain embodiments, the first binding domain that binds to TRGV9 is an isolated antibody. In certain embodiments, the first binding domain that binds to TRGV9 is an intact antibody. In some embodiments, the first binding domain that binds to TRGV9 is an IgG antibody. In some embodiments, the first binding domain that binds to TRGV9 is an IgG1 antibody. In some embodiments, the first binding domain that binds to TRGV9 is an IgG2 antibody. In some embodiments, the first binding domain that binds to TRGV9 is an IgG3 antibody. In some embodiments, the first binding domain that binds to TRGV9 is an IgG4 antibody. In some embodiments, the first binding domain that binds to TRGV9 comprises a kappa light chain. In some embodiments, the first binding domain that binds to TRGV9 comprises a lambda light chain. In some embodiments, the first binding domain that binds to TRGV9 is a monoclonal antibody. In some embodiments, the first binding domain that binds to TRGV9 is multivalent. In some embodiments, the first binding domain that binds to TRGV9 is capable of binding at least three antigens. In some embodiments, the first binding domain that binds to TRGV9 is capable of binding at least four antigens. In some embodiments, the first binding domain that binds to TRGV9 is capable of binding at least five antigens. In some embodiments, the first binding domain that binds to TRGV9 is a multispecific antibody. In some embodiments, the first binding domain that binds to TRGV9 is a bispecific antibody. In some embodiments, the first binding domain that binds to TRGV9 is a trispecific antibody. In some embodiments, the first binding domain that binds to TRGV9 is a quadraspecific antibody. In other embodiments, the first binding domain that binds to TRGV9 is an antigen binding fragment. In some embodiments, the antigen binding fragment is a functional fragment. In some embodiments, the antigen binding fragment is chimeric. In some embodiments, the antigen binding fragment is human. In some embodiments, a antigen binding fragment is humanized. In certain embodiments, the antigen binding fragment is an isolated antigen binding fragment.

In some embodiments, the second binding domain that binds to the second target is chimeric. In some embodiments, the second binding domain that binds to the second target is human. In some embodiments, the second binding domain that binds to the second target is humanized. In certain embodiments, the second binding domain that binds to the second target is an isolated antibody. In certain embodiments, the second binding domain that binds to the second target is an intact antibody. In some embodiments, the second binding domain that binds to the second target is an IgG antibody. In some embodiments, the second binding domain that binds to the second target is an IgG1 antibody. In some embodiments, the second binding domain that binds to the second target is an IgG2 antibody. In some embodiments, the second binding domain that binds to the second target is an IgG3 antibody. In some embodiments, the second binding domain that binds to the second target is an IgG4 antibody. In some embodiments, the second binding domain that binds to the second target comprises a kappa light chain. In some embodiments, the second binding domain that binds to the second target comprises a lambda light chain. In some embodiments, the second binding domain that binds to the second target is a monoclonal antibody. In some embodiments, the second binding domain that binds to the second target is multivalent. In some embodiments, the second binding domain that binds to the second target is capable of binding at least three antigens. In some embodiments, the second binding domain that binds to the second target is capable of binding at least four antigens. In some embodiments, the second binding domain that binds to the second target is capable of binding at least five antigens. other embodiments, the second binding domain that binds to the second target is an antigen binding fragment. In some embodiments, the antigen binding fragment is a functional fragment. In some embodiments, the antigen binding fragment is chimeric. In some embodiments, the antigen binding fragment is human. In some embodiments, an antigen binding fragment is humanized. In certain embodiments, the antigen binding fragment is an isolated antigen binding fragment.

In certain embodiments, the multispecific antibody comprises any of the TRGV9 antibodies provided herein. In some embodiments, the multispecific antibody comprises an antigen binding fragment of any of the TRGV9 antibodies provided herein.

In certain embodiments, the multispecific antibody comprises any of the CD123 antibodies provided herein. In some embodiments, the multispecific antibody comprises an antigen binding fragment of any of the CD123 antibodies provided herein. In other embodiments, the multispecific antibody comprises any of the TRGV9 antibodies provided herein, and any of the CD123 antibodies provided herein. In some embodiments, the multispecific antibody comprises any of the TRGV9 antibodies provided herein, and an antigen binding fragment of any of the CD123 antibodies provided herein. In other embodiments, the multispecific antibody comprises an antigen binding fragment any of the TRGV9 antibodies provided herein, and any of the CD123 antibodies provided herein. In yet other embodiments, the multispecific antibody comprises an antigen binding fragment of any of the TRGV9 antibodies provided herein, and an antigen binding fragment of any of the CD123 antibodies provided herein.

In certain embodiments, the multispecific antibody comprises any of the CD33 antibodies provided herein. In some embodiments, the multispecific antibody comprises an antigen binding fragment of any of the CD33 antibodies provided herein. In other embodiments, the multispecific antibody comprises any of the TRGV9 antibodies provided herein, and any of the CD33 antibodies provided herein. In some embodiments, the multispecific antibody comprises any of the TRGV9 antibodies provided herein, and an antigen binding fragment of any of the CD33 antibodies provided herein. In other embodiments, the multispecific antibody comprises an antigen binding fragment any of the TRGV9 antibodies provided herein, and any of the CD33 antibodies provided herein. In yet other embodiments, the multispecific antibody comprises an antigen binding fragment of any of the TRGV9 antibodies provided herein, and an antigen binding fragment of any of the CD33 antibodies provided herein.

In certain embodiments, the multispecific antibody comprises any of the TRBC1 antibodies provided herein. In some embodiments, the multispecific antibody comprises an antigen binding fragment of any of the TRBC1 antibodies provided herein. In other embodiments, the multispecific antibody comprises any of the TRGV9 antibodies provided herein, and any of the TRBC1 antibodies provided herein. In some embodiments, the multispecific antibody comprises any of the TRGV9 antibodies provided herein, and an antigen binding fragment of any of the TRBC1 antibodies provided herein. In other embodiments, the multispecific antibody comprises an antigen binding fragment any of the TRGV9 antibodies provided herein, and any of the TRBC1 antibodies provided herein. In yet other embodiments, the multispecific antibody comprises an antigen binding fragment of any of the TRGV9 antibodies provided herein, and an antigen binding fragment of any of the TRBC1 antibodies provided herein.

In certain embodiments, the multispecific antibody comprises any of the BCMA antibodies provided herein. In some embodiments, the multispecific antibody comprises an antigen binding fragment of any of the BCMA antibodies provided herein. In other embodiments, the multispecific antibody comprises any of the TRGV9 antibodies provided herein, and any of the BCMA antibodies provided herein. In some embodiments, the multispecific antibody comprises any of the TRGV9 antibodies provided herein, and an antigen binding fragment of any of the BCMA antibodies provided herein. In other embodiments, the multispecific antibody comprises an antigen binding fragment any of the TRGV9 antibodies provided herein, and any of the BCMA antibodies provided herein. In yet other embodiments, the multispecific antibody comprises an antigen binding fragment of any of the TRGV9 antibodies provided herein, and an antigen binding fragment of any of the BCMA antibodies provided herein.

In certain embodiments, the multispecific antibody comprises any of the PSMA antibodies provided herein. In some embodiments, the multispecific antibody comprises an antigen binding fragment of any of the PSMA antibodies provided herein. In other embodiments, the multispecific antibody comprises any of the TRGV9 antibodies provided herein, and any of the PSMA antibodies provided herein. In some embodiments, the multispecific antibody comprises any of the TRGV9 antibodies provided herein, and an antigen binding fragment of any of the PSMA antibodies provided herein. In other embodiments, the multispecific antibody comprises an antigen binding fragment any of the TRGV9 antibodies provided herein, and any of the PSMA antibodies provided herein. In yet other embodiments, the multispecific antibody comprises an antigen binding fragment of any of the TRGV9 antibodies provided herein, and an antigen binding fragment of any of the PSMA antibodies provided herein.

In another aspect, provided herein is an antibody that competes for binding to TRGV9 with any of the TRGV9 antibodies described herein. In another aspect, provided herein is an antibody that binds to the same epitope as any of the TRGV9 antibodies described herein. In another aspect, provided is a TRGV9 antibody that binds an epitope on TRGV9 that overlaps with the epitope on TRGV9 bound by a TRGV9 antibody described herein. In some embodiments, the TRGV9 antibody comprises a VH CDR1, VH CDR2, and VH CDR3 of a TRGV9 antibody provided herein. In some embodiments, the TRGV9 antibody comprises a VL CDR1, VL CDR2, and VL CDR3 of a TRGV9 antibody provided herein. In some embodiments, the TRGV9 antibody comprises a VH CDR1, VH CDR2, VH CDR3, a VL CDR1, VL CDR2, and VL CDR3 of a TRGV9 antibody provided herein. In some embodiments, the TRGV9 antibody comprises a VH of a TRGV9 antibody provided herein. In some embodiments, the TRGV9 antibody comprises a VL of a TRGV9 antibody provided herein. In some embodiments, the TRGV9 antibody comprises a VH and a VL of a TRGV9 antibody provided herein. In some embodiments, the TRGV9 antibody comprises a VH CDR1, VH CDR2, VH CDR3, a VL CDR1, VL CDR2, and VL CDR3 of a TRGV9 antibody provided herein. In some embodiments, the VH CDR1, VH CDR2, VH CDR3, VL CDR1, VL CDR2, and VL CDR3 amino acid sequences of the TRGV9 antibody are according to the Kabat numbering system. In some embodiments, the VH CDR1, VH CDR2, VH CDR3, VL CDR1, VL CDR2, and VL CDR3 amino acid sequences of the TRGV9 antibody are according to the Chothia numbering system. In some embodiments, the VH CDR1, VH CDR2, VH CDR3, VL CDR1, VL CDR2, and VL CDR3 amino acid sequences of the TRGV9 antibody are according to the AbM numbering system. In some embodiments, the VH CDR1, VH CDR2, VH CDR3, VL CDR1, VL CDR2, and VL CDR3 amino acid sequences of the TRGV9 antibody are according to the Contact numbering system. In some embodiments, the VH CDR1, VH CDR2, VH CDR3, VL CDR1, VL CDR2, and VL CDR3 amino acid sequences of the TRGV9 antibody are according to the IMGT numbering system. In certain embodiments, the TRGV9 antibody is a multispecific antibody. In some embodiments, the TRGV9 antibody is a bispecific antibody.

In another aspect, provided is an antibody that competes for binding to TRGV9 with a TRGV9 reference antibody. In another aspect, provided is a TRGV9 antibody that binds to the same TRGV9 epitope as a TRGV9 reference antibody. In another aspect, provided is a TRGV9 antibody that binds an epitope on TRGV9 that overlaps with the epitope on TRGV9 bound by a TRGV9 reference antibody. In some embodiments, the TRGV9 reference antibody comprises a VH CDR1, VH CDR2, and VH CDR3 of a TRGV9 reference antibody provided herein. In some embodiments, the TRGV9 reference antibody comprises a VL CDR1, VL CDR2, and VL CDR3 of a TRGV9 reference antibody provided herein. In some embodiments, the TRGV9 reference antibody comprises a VH CDR1, VH CDR2, VH CDR3, a VL CDR1, VL CDR2, and VL CDR3 of a TRGV9 reference antibody provided herein. In some embodiments, the TRGV9 reference antibody comprises a VH of a TRGV9 reference antibody provided herein. In some embodiments, the TRGV9 reference antibody comprises a VL of a TRGV9 reference antibody provided herein. In some embodiments, the TRGV9 reference antibody comprises a VH and a VL of a TRGV9 reference antibody provided herein. In some embodiments, the TRGV9 reference antibody comprises a VH CDR1, VH CDR2, VH CDR3, a VL CDR1, VL CDR2, and VL CDR3 of a TRGV9 reference antibody provided herein. In some embodiments, the VH CDR1, VH CDR2, VH CDR3, VL CDR1, VL CDR2, and VL CDR3 amino acid sequences of the TRGV9 reference antibody are according to the Kabat numbering system. In some embodiments, the VH CDR1, VH CDR2, VH CDR3, VL CDR1, VL CDR2, and VL CDR3 amino acid sequences of the TRGV9 reference antibody are according to the Chothia numbering system. In some embodiments, the VH CDR1, VH CDR2, VH CDR3, VL CDR1, VL CDR2, and VL CDR3 amino acid sequences of the TRGV9 reference antibody are according to the AbM numbering system. In some embodiments, the VH CDR1, VH CDR2, VH CDR3, VL CDR1, VL CDR2, and VL CDR3 amino acid sequences of the TRGV9 reference antibody are according to the Contact numbering system. In some embodiments, the VH CDR1, VH CDR2, VH CDR3, VL CDR1, VL CDR2, and VL CDR3 amino acid sequences of the TRGV9 reference antibody are according to the IMGT numbering system. In certain embodiments, the antibody is a multispecific antibody. In some embodiments, the antibody is a bispecific antibody. In certain embodiments, the TRGV9 reference antibody is a multispecific antibody. In some embodiments, the TRGV9 reference antibody is a bispecific antibody.

The term “compete” when used in the context of TRGV9 antibodies (e.g., TRGV9 antibodies that bind to TRGV9 and compete for the same epitope or binding site on a target) means competition as determined by an assay in which the antibody (or binding fragment) thereof under study prevents or inhibits the specific binding of a reference molecule (e.g., a reference ligand or reference antigen-binding protein, such as a reference antibody) to a common antigen (e.g., TRGV9 or a fragment thereof). Numerous types of competitive binding assays can be used to determine if a test antibody competes with a reference antibody for binding to TRGV9 (e.g., human TRGV9). Examples of assays that can be employed include solid phase direct or indirect RIA, solid phase direct or indirect enzyme immunoassay (EIA), sandwich competition assay (see, e.g., Stahli et al., 1983, Methods in Enzymology 9:242-53), solid phase direct biotin-avidin EIA (see, e.g., Kirkland et al., 1986, J. Immunol. 137:3614-19), solid phase direct labeled assay, solid phase direct labeled sandwich assay (see, e.g., Harlow and Lane, Antibodies, A Laboratory Manual (1988)), solid phase direct label RIA using 1-125 label (see, e.g., Morel et al., 1988, Mol. Immunol. 25:7-15), and direct labeled MA (Moldenhauer et al., 1990, Scand. J. Immunol. 32:77-82). Typically, such an assay involves the use of a purified antigen (e.g., TRGV9 such as human TRGV9) bound to a solid surface, or cells bearing either of an unlabeled test antigen-binding protein (e.g., test TRGV9 antibody) or a labeled reference antigen-binding protein (e.g., reference TRGV9 antibody). Competitive inhibition may be measured by determining the amount of label bound to the solid surface or cells in the presence of the test antigen-binding protein. Usually the test antigen-binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur. Additional details regarding methods for determining competitive binding are described herein. Usually, when a competing antibody protein is present in excess, it will inhibit specific binding of a reference antibody to a common antigen by at least 30%, for example 40%, 45%, 50%, 55%, 60%, 65%, 70%, or 75%. In some instance, binding is inhibited by at least 80%, 85%, 90%, 95%, 96%, 97%, 98%, 99%, or more.

An antibody binds “an epitope,” “essentially the same epitope,” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent, or enzyme labels.

“Epitope mapping” is the process of identifying the binding sites, or epitopes, of antibodies on their target antigens. “Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

In specific embodiments, TRGV9 antibodies provided herein share the common feature of competing with each other for the binding of TRGV9. This competitive inhibition can indicate that each antibody binds to the same region of TRGV9 (e.g., the same epitope), thereby asserting similar effects. In certain embodiments, TRGV9 antibodies provided herein include L7A5_1 (TRGV9_1), L7A5_2 (TRGV9_2), L7A5_3 (TRGV9_3), L7A5_4 (TRGV9_4), TRGV9Ab_var17, TRGV9Ab_var29, VG9_B3_RN, VG9B420, VG9SB10SC1087_P18_D08, VG9SB10SC1087_P18_C12, or VG9SB10SC1087_P19_C03, or those derived from or based on these antibodies. In other embodiments, TRGV9 antibodies provided herein compete for binding with an antibody that is, or derived from, or based on L7A5_1 (TRGV9_1). In other embodiments, TRGV9 antibodies provided herein compete for binding with an antibody that is, or derived from, or based on L7A5_2 (TRGV9_2). In other embodiments, TRGV9 antibodies provided herein compete for binding with an antibody that is, or derived from, or based on L7A5_3 (TRGV9_3). In other embodiments, TRGV9 antibodies provided herein compete for binding with an antibody that is, or derived from, or based on L7A5_4 (TRGV9_4). In other embodiments, TRGV9 antibodies provided herein compete for binding with an antibody that is, or derived from, or based on TRGV9Ab_var17. In other embodiments, TRGV9 antibodies provided herein compete for binding with an antibody that is, or derived from, or based on TRGV9Ab_var29. In other embodiments, TRGV9 antibodies provided herein compete for binding with an antibody that is, or derived from, or based on VG9_B3_RN. In other embodiments, TRGV9 antibodies provided herein compete for binding with an antibody that is, or derived from, or based on VG9B420. In other embodiments, TRGV9 antibodies provided herein compete for binding with an antibody that is, or derived from, or based on VG9SB10SC1087_P18_D08. In other embodiments, TRGV9 antibodies provided herein compete for binding with an antibody that is, or derived from, or based on VG9SB10SC1087_P18_C12. In other embodiments, TRGV9 antibodies provided herein compete for binding with an antibody that is, or derived from, or based on or VG9SB10SC1087_P19_C03. In some embodiments, the TRGV9 antibodies have CDR sequences as provided herein, including the Sequence Listing and tables provided herein.

In certain embodiments, the TRGV9 antibodies bind to a specific domain or epitope of human TRGV9 (e.g., residues 49 to 68 of the human TRGV9 sequence of SEQ ID NO:789 (L49VSISYDGTVRKESGIPSGK68 (SEQ ID NO:774))); see also FIG. 27A, Example 8.2). Taken together, the results described herein demonstrate that the effects observed for a TRGV9 antibody that is, is derived from, or is based on a TRGV9 antibody provided herein, including an antibody having one or more CDRs described in the Sequence Listing and Tables 1-39, can be extrapolated to other TRGV9 antibodies described herein having the same or similar epitope specificity (e.g., the same or similar CDRs). For example, the activities of antibodies as shown in the Examples, for an exemplary TRGV9 antibodies, are representative of the activities and effects of other TRGV9 antibodies described herein.

In yet another aspect, antibodies are provided that compete with one of the exemplified antibodies or functional fragments for binding to TRGV9. Such antibodies may also bind to the same epitope as one of the herein exemplified antibodies, or an overlapping epitope. Antibodies and fragments that compete with or bind to the same epitope as the exemplified antibodies are expected to show similar functional properties. The exemplified antigen-binding proteins and fragments include those with the VH and VL regions, and CDRs provided herein, including those in the Sequence Listing and Tables 1-39. Thus, as a specific example, the antibodies that are provided include those that compete with an antibody comprising: (a) 1, 2, 3, 4, 5, or all 6 of the CDRs listed for a TRGV9 antibody provided herein; (b) a VH and a VL selected from the VH and the VL regions listed for a TRGV9 antibody provided herein; or (c) two light chains and two heavy chains comprising a VH and a VL as specified for a TRGV9 antibody provided herein.

In some embodiments, the antibody is L7A5_1 (TRGV9_1). In some embodiments, the antibody is L7A5_2 (TRGV9_2). In some embodiments, the antibody is L7A5_3 (TRGV9_3). In some embodiments, the antibody is L7A5_4 (TRGV9_4). In some embodiments, the antibody is TRGV9Ab_var17. In some embodiments, the antibody is TRGV9Ab_var29. In some embodiments, the antibody is VG9_B3_RN. In some embodiments, the antibody is VG9B420. In some embodiments, the antibody is VG9SB10SC1087_P18_D08. In some embodiments, the antibody is VG9SB10SC1087_P18_C12. In some embodiments, the antibody is or VG9SB10SC1087_P19_C03.

In another aspect, antibodies (including antigen-binding fragments thereof) provided herein bind to a region, including an epitope, of human TRGV9. For example, in some embodiments, an antibody provided herein binds to a region of human TRGV9 (see FIG. 27A) comprising amino acid residues 49 to 68 of human TRGV9 (L₄₉VSISYDGTVRKESGIPSGK68 (SEQ ID NO:774)). In still another aspect, antibodies provided herein bind to a specific epitope of human TRGV9. In certain embodiments, the antibody or antigen-binding fragment thereof, when bound to human TRGV9, binds to at least one of residues 49 to 68 (L₄₉VSISYDGTVRKESGIPSGK₆₈ (SEQ ID NO:774)) within an amino acid sequence of human TRGV9 (see FIG. 27A).

In some embodiments, the TRGV9 antibody binds to at least one residue selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to two or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to three or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to four or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to five or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to six or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to seven or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to eight or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to nine or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to ten or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to eleven or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to twelve or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to thirteen or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to fourteen or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to fifteen or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to sixteen or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to seventeen or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to eighteen or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to nineteen or more residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A). In some embodiments, the TRGV9 antibody binds to all twenty residues selected from the group consisting of L49, V50, S51, I52, S53, Y54, D55, G56, T57, V58, R59, K60, E61, S62, G63, 164, P65, S66, G67 and K68 within an amino acid sequence of human TRGV9 (see FIG. 27A).

In one embodiment, the TRGV9 antibody binds L49 within an amino acid sequence of human TRGV9 (see FIG. 27A). In one embodiment, the TRGV9 antibody binds V50 within an amino acid sequence of human TRGV9 (see FIG. 27A). In one embodiment, the TRGV9 antibody binds S51 within an amino acid sequence of human TRGV9 (see FIG. 27A). In one embodiment, the TRGV9 antibody binds 152 within an amino acid sequence of human TRGV9 (see FIG. 27A). In one embodiment, the TRGV9 antibody binds S53 within an amino acid sequence of human TRGV9 (see FIG. 27A). In one embodiment, the TRGV9 antibody binds Y54 within an amino acid sequence of human TRGV9 (see FIG. 27A). In one embodiment, the TRGV9 antibody binds D55 within an amino acid sequence of human TRGV9 (see FIG. 27A). In one embodiment, the TRGV9 antibody binds G56 within an amino acid sequence of human TRGV9 (see FIG. 27A). In one embodiment, the TRGV9 antibody binds T57 within an amino acid sequence of human TRGV9 (see FIG. 27A). In one embodiment, the TRGV9 antibody binds V58 within an amino acid sequence of human TRGV9 (see FIG. 27A). In one embodiment, the TRGV9 antibody binds R59 within an amino acid sequence of human TRGV9 (see FIG. 27A). In one embodiment, the TRGV9 antibody binds K60 within an amino acid sequence of human TRGV9 (see FIG. 27A). In one embodiment, the TRGV9 antibody binds E61 within an amino acid sequence of human TRGV9 (see FIG. 27A). In one embodiment, the TRGV9 antibody binds S62 within an amino acid sequence of human TRGV9 (see FIG. 27A). In one embodiment, the TRGV9 antibody binds G63 within an amino acid sequence of human TRGV9 (see FIG. 27A). In one embodiment, the TRGV9 antibody binds 164 within an amino acid sequence of human TRGV9 (see FIG. 27A). In one embodiment, the TRGV9 antibody binds P65 within an amino acid sequence of human TRGV9 (see FIG. 27A). In one embodiment, the TRGV9 antibody binds S66 within an amino acid sequence of human TRGV9 (see FIG. 27A). In one embodiment, the TRGV9 antibody binds G67 within an amino acid sequence of human TRGV9 (see FIG. 27A). In one embodiment, the TRGV9 antibody binds K68 within an amino acid sequence of human TRGV9 (see FIG. 27A). Any combination of two, three, four, five, six, seven, eight, nine, ten, eleven, twelve, thirteen, fourteen, fifteen, sixteen, seventeen, eighteen, nineteen, or twenty of the above-referenced amino acid TRGV9 binding sites is also contemplated.

In certain embodiments, a TRGV9 antibody comprises a paratope of a TRGV9 antibody provided herein. In certain embodiments, a multispecific TRGV9 antibody comprises a paratope of a TRGV9 antibody provided herein. Exemplary paratopes are provided in FIG. 27B. In one embodiment the paratope comprises amino acids 26 to 36 of SEQ ID NO:7. In one embodiment the paratope comprises amino acids 45 to 50 of SEQ ID NO:7. In one embodiment the paratope comprises amino acids 94 to 96 of SEQ ID NO:7. In one embodiment the paratope comprises amino acids 26 to 36, 45 to 50 and 94 to 96 of SEQ ID NO:7. In one embodiment the paratope comprises amino acids 29 to 41 of SEQ ID NO:8. In one embodiment the paratope comprises amino acids 52 to 60 of SEQ ID NO:8. In one embodiment the paratope comprises amino acids 94 to 96 of SEQ ID NO:8. In one embodiment the paratope comprises amino acids 29 to 41, 52 to 60, and 94 to 96 of SEQ ID NO:8.

The multispecific TRGV9 antibodies provided herein can be engineered into various well-known antibody forms. In certain embodiments, the multispecific TRGV9 antibody is a bispecific TRGV9 antibody. In some embodiments, the bispecific antibody is a diabody. In some embodiments, the bispecific antibody is a cross-body. In some embodiments, the bispecific antibody is a diabody, a cross-body, or a bispecific antibody obtained via a controlled Fab arm exchange as those described herein.

In some embodiments, the multispecific antibodies comprise IgG-like molecules with complementary CH3 domains that promote heterodimerization. In some embodiments, the multispecific antibodies comprise recombinant IgG-like dual targeting molecules, wherein the two sides of the molecule each contain the Fab fragment or part of the Fab fragment of at least two different antibodies. In some embodiments, the multispecific antibodies comprise IgG fusion molecules, wherein full length IgG antibodies are fused to an extra Fab fragment or parts of Fab fragment. In some embodiments, the multispecific antibodies comprise Fc fusion molecules, wherein single chain Fv molecules or stabilized diabodies are fused to heavy-chain constant-domains, Fc-regions or parts thereof. In some embodiments, the multispecific antibodies comprise Fab fusion molecules, wherein different Fab-fragments are fused together. In some embodiments, the multispecific antibodies comprise scFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule. In certain embodiments, the multispecific antibodies are bispecific antibodies.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus) and the DuoBody (Genmab A/S). In certain embodiments, the multispecific antibody is in a Triomab/Quadroma (Trion Pharma/Fresenius Biotech) format. In certain embodiments, the multispecific antibody is in a Knobs-into-Hole (Genentech) format. In certain embodiments, the multispecific antibody is in a CrossMAb (Roche) format. In certain embodiments, the multispecific antibody is in an electrostatically-matched (Amgen) format. In certain embodiments, the multispecific antibody is in a LUZ-Y (Genentech) format. In certain embodiments, the multispecific antibody is in a Strand Exchange Engineered Domain body (SEEDbody) (EMD Serono) format. In certain embodiments, the multispecific antibody is in a Biclonic (Merus) format. In certain embodiments, the multispecific antibody is in a DuoBody (Genmab A/S) format.

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine-China).

In some embodiments, Fab fusion bispecific antibodies include F(ab)₂ (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al., (2015) Drug Discovery Today 20(5):588-594.

Bispecific antibodies provided herein can comprise antibodies having a full length antibody structure. “Full length antibody” refers to an antibody having two full length antibody heavy chains and two full length antibody light chains. A full length antibody heavy chain (HC) consists of well-known heavy chain variable and constant domains VH, CH1, hinge, CH2, and CH3. A full length antibody light chain (LC) consists of well-known light chain variable and constant domains VL and CL. The full length antibody can be lacking the C-terminal lysine (K) in either one or both heavy chains. “Fab-arm” or “half molecule” refers to one heavy chain-light chain pair that specifically binds an antigen.

Full length bispecific antibodies can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules which each binding a distinct epitope, i.e. an epitope on TRGV9 and an epitope on a second target antigen (e.g., not TRGV9). Other methods of making multispecific antibodies are known and contemplated.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

As mentioned elsewhere, in some embodiments, the bispecific antibodies include designs such as the Triomab/Quadroma (Trion Pharma/Fresenius Biotech), Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), the Biclonic (Merus) and the DuoBody (Genmab A/S).

In some embodiments, a TRGV9 multispecific antibody provided herein is in the knob-and-hole format. In some embodiments, a TRGV9 multispecific antibody provided herein is in a DuoBody format.

The Triomab quadroma technology can be used to generate full length bispecific antibodies provided herein. Triomab technology promotes Fab arm exchange between two parental chimeric antibodies, one parental mAb having IgG2a and the second parental mAb having rat IgG2b constant regions, yielding chimeric bispecific antibodies.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

The CrossMAb technology can be used to generate full length bispecific antibodies provided herein. CrossMAbs, in addition to utilizing the “knob-in-hole” strategy to promoter Fab arm exchange, have in one of the half arms the CH1 and the CL domains exchanged to ensure correct light chain pairing of the resulting bispecific antibody (see e.g. U.S. Pat. No. 8,242,247).

Other cross-over strategies can be used to generate full length bispecific antibodies provided herein by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V_K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849.

LUZ-Y technology can be utilized to generate bispecific antibodies provided herein. In this technology, a leucine zipper is added into the C terminus of the CH3 domains to drive the heterodimer assembly from parental mAbs that is removed post-purification as described in Wranik et al., (2012) J Biol Chem 287(52): 42221-9.

SEEDbody technology can be utilized to generate bispecific antibodies provided herein. SEEDbodies have, in their constant domains, select IgG residues substituted with IgA residues to promote heterodimerszation as described in U.S. Patent No. US20070287170.

In addition to methods described above, bispecific antibodies of the invention can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody and the second monospecific bivalent antibody are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, preferably a reducing agent selected from the group consisting of: 2-mercaptoethylamine, dithiothreitol and tris (2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 can be used.

In specific embodiments, the linker is a peptide linker. In some embodiments, the liker comprises a naturally occurring amino acid. Exemplary amino acids that can be included into the linker are Gly, Ser Pro, Thr, Glu, Lys, Arg, Ile, Leu, His and The. In some embodiments, the linker has a length that is adequate to link the VH and the VL in such a way that they form the correct conformation relative to one another so that they retain the desired activity, such as binding to the target (e.g., TRGV9).

In certain embodiments, the linker is about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long. Exemplary linkers that can be used are Gly rich linkers, Gly and Ser containing linkers, Gly and Ala containing linkers, Ala and Ser containing linkers, and other flexible linkers.

Any of the VH and the VL domains identified herein (e.g., those that bind TRGV9) can be engineered into scFv format. In some embodiments, the scFv format is in the VH-linker-VL orientation. In other embodiments, the scFv format is in the VL-linker-VH orientation. Any of the VH and the VL domains identified herein can also be used to generate sc(Fv)2 structures. In some embodiments, the sc(Fv)2 structure is VH-linker-VL-linker-VL-linker-VH. In some embodiments, the sc(Fv)2 structure is VH-linker-VL-linker-VH-linker-VL. In some embodiments, the sc(Fv)2 structure is VH-linker-VH-linker-VL-linker-VL. In some embodiments, the sc(Fv)2 structure is VL-linker-VH-linker-VH-linker-VL. In some embodiments, the sc(Fv)2 structure is VL-linker-VH-linker-VL-linker-VH. In some embodiments, the sc(Fv)2 structure is VL-linker-VL-linker-VH-linker-VH. In some embodiments, the scFv comprises, from the N- to C-terminus, a VH, a first linker (L1) and a VL (VH-L1-VL). In other embodiments, the scFv comprises, from the N- to C-terminus, the VL, the L1 and the VH (VL-L1-VH). In certain embodiments, antibodies provided herein comprise two linkers. In other embodiments, antibodies provided herein comprise three linkers. In yet other embodiments, antibodies provided herein comprise four or more linkers. In certain embodiments, the antibody is an antigen binding fragment thereof.

According to another particular aspect, provided herein is a TRGV9 antibody that induces antibody-dependent cell-mediated cytotoxicity (ADCC). The antibody or antigen-binding fragment thereof can, for example, induce ADCC in vitro. In some embodiments, the second target is present on the surface of a target cell. In some embodiments, the target cell expressing the second target is killed when the multispecific TRGV9 antibody binds to TRGV9 on the surface of a T cell and the second target. In a specific embodiment, the T cell is a γδ T cell. In some embodiments, the target cell is a cancer cell.

In certain embodiments, the TRGV9 antibody induces T cell dependent cytotoxicity of the target cell in vitro with an EC₅₀ of less than about 160 pM, when assessed in vitro at an effector to target cell ratio of 1:1.

In one such embodiment, the multispecific antibody is an isolated TRGV9×CD123 bispecific antibody or antigen-binding fragment thereof that exhibits an EC₅₀ of less than about 160 pM, when assessed in vitro with a mixture of γδ T effector cells and Kasumi3 AML target cells, where such cells are present in an effector to target cell ratio of about 1:1 and the bispecific antibody or antigen-binding fragment thereof is present at a concentration of about 30 ng/mL.

In some embodiments, the TRGV9 antibody induces T cell dependent cytotoxicity of the target cell in vitro with an EC₅₀ of less than about 500 pM. In some embodiments, the multispecific antibody induces T cell dependent cytotoxicity of the target cell in vitro with an EC₅₀ of less than about 300 pM. In some embodiments, the multispecific antibody induces T cell dependent cytotoxicity of the target cell in vitro with an EC₅₀ of less than about 160 pM. In some embodiments, the EC₅₀ is assessed with a mixture of effector T cells and target cells expressing the second target. In some embodiments, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1. In a specific embodiments, the T cell is a γδ T cell.

In certain embodiments, the EC₅₀ is less than about 1000 pM, less than about 900 pM, less than about 800 pM, less than about 700 pM, less than about 600 pM, less than about 500 pM, less than about 400 pM, less than about 300 pM, less than about 200 pM, less than about 190 pM, less than about 180 pM, less than about 170 pM, less than about 160 pM, less than about 150 pM, less than about 140 pM, less than about 130 pM, less than about 120 pM, less than about 110 pM, less than about 100 pM, less than about 90 pM, less than about 80 pM, less than about 70 pM, less than about 60 pM, less than about 50 pM, less than about 40 pM, less than about 30 pM, less than about 20 pM, or less than about 10 pM.

In certain embodiments, the effector to target cell ratio is 0.01:1, 0.02:1, 0.03:1, 0.04:1, 0.05:1, 0.06:1, 0.07:1, 0.08:1, 0.09:1, 1:1, 2:1, 3:1, 4:1, 5:1, 6:1, 7:1, 8:1, 9:1, or 10:1.

In certain embodiments, the concentration of the multispecific antibody thereof is about 0.000005 ng/mL, about 0.00005 ng/mL, about 0.0005, about 0.005 ng/mL, about 0.01 ng/mL, about 0.02 ng/mL, about 0.03 ng/mL, about 0.04 ng/mL, about 0.05 ng/mL, about 0.06 ng/mL, about 0.07 ng/mL, about 0.08 ng/mL, about 0.09 ng/mL, about 0.1 ng/mL, about 0.5 ng/mL, about 1.0 ng/mL, about 10 ng/mL, about 20 ng/mL about, about 30 ng/mL about 40 ng/mL, about 50 ng/mL, about 60 ng/mL, about 70 ng/mL, about 80 ng/mL, about 90 ng/mL, about 100 ng/mL, or about 1000 ng/mL.

In some embodiments described herein, immune effector properties of the TRGV9 antibodies provided herein can be enhanced or silenced through Fc modifications by techniques known to those skilled in the art. For example, Fc effector functions such as Clq binding, complement dependent cytotoxicity (CDC), ADCC, antibody-dependent cell-mediated phagocytosis (ADCP), down regulation of cell surface receptors (e.g., B cell receptor; BCR), etc. can be provided and/or controlled by modifying residues in the Fc responsible for these activities.

“Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

The ability of antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such Abs can be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the α-1,6-fucosyltrasferase (FUT8) gene (Mori et al., Biotechnol Bioeng 88:901-908, 2004), or coexpression of β-1,4-N-acetylglucosaminyltransferase III and golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments described herein, ADCC elicited by the anti-TRGV9 antibodies provided herein can also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

Also provided is a nucleic acid encoding a TRGV9 antibody provided herein. In another general aspect, provide is a vector comprising an isolated nucleic acid encoding a TRGV9 antibody provided herein. In another general aspect, provided is a vector comprising an isolated nucleic acid encoding a TRGV9 antibody provided herein. Also provided is a vector comprising a nucleic acid encoding a TRGV9 antibody provided herein. Also provided is a host cell comprising a vector comprising a nucleic acid encoding a TRGV9 antibody provided herein. Also provided is a kit comprising the vector comprising a nucleic acid encoding a TRGV9 antibody provided herein, and packaging for the same. In another general aspect, provided herein is an isolated nucleic acid encoding a TRGV9 monoclonal antibody or antigen-binding fragment thereof provided herein. In certain embodiments, the antibody is a multispecific TRGV9 antibody. Also provided is a nucleic acid encoding a multispecific TRGV9 antibody comprising: (a) a first binding domain that binds to TRGV9, and (b) a second binding domain that binds to a second target that is not TRGV9, as provided herein. In some embodiments, the antibody is a multispecific TRGV9×CD123 antibody. In some embodiments, the antibody is a multispecific TRGV9×CD33 antibody. In some embodiments, the antibody is a multispecific TRGV9×TRBC1 antibody. In some embodiments, the antibody is a multispecific TRGV9×BCMA antibody. In some embodiments, the antibody is a multispecific TRGV9×PSMA antibody. In a specific embodiment, the multispecific antibody is a bispecific antibody. In some embodiments, the TRGV9 antibody is a TRGV9 antigen binding fragment.

It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies and/or bispecific antibodies of the invention can be altered without changing the amino acid sequences of the proteins.

Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention. Such techniques are well known to those skilled in the art in view of the present disclosure.

Any host cell known to those skilled in the art in view of the present disclosure can be used for recombinant expression of antibodies or antigen-binding fragments thereof the invention. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, provided is a method of producing a TRGV9 antibody disclosed herein. In some embodiments, the method comprises culturing a cell comprising a nucleic acid encoding the TRGV9 antibody under conditions to produce a TRGV9 antibody disclosed herein and recovering the TRGV9 antibody from the cell or cell culture (e.g., from the supernatant). Expressed antibodies can be harvested from the cells and purified according to conventional techniques known in the art and as described herein.

Pharmaceutical Compositions

In another general aspect, provided is a pharmaceutical composition comprising a TRGV9 antibody provided herein and a pharmaceutically acceptable carrier. In certain embodiments, the antibody is isolated. Also provided is a method of producing the pharmaceutical composition, comprising combining the antibody with a pharmaceutically acceptable carrier to obtain the pharmaceutical composition.

In another general aspect, provided is a pharmaceutical composition comprising a TRGV9 multispecific antibody provided herein and a pharmaceutically acceptable carrier. In certain embodiments, the multispecific antibody is isolated. Also provided is a method of producing the pharmaceutical composition, comprising combining the multispecific antibody with a pharmaceutically acceptable carrier to obtain the pharmaceutical composition. In another aspect, provided herein is a pharmaceutical composition comprising a comprising: (a) a first binding domain that binds to TRGV9, and (b) a second binding domain that binds to a second target, and a pharmaceutically acceptable carrier. Any of the multispecific antibodies provided herein are contemplated in the pharmaceutical compositions. In certain embodiments, the second binding domain binds to CD123. In certain embodiments, the second binding domain binds to CD33. In certain embodiments, the second binding domain binds to TRBC1. In certain embodiments, the second binding domain binds to BCMA. In certain embodiments, the second binding domain binds to PSMA. Any of the antibodies provided herein are contemplated in the pharmaceutical compositions.

The term “pharmaceutical composition” as used herein means a product comprising an antibody provided herein together with a pharmaceutically acceptable carrier. Antibodies of provided herein and compositions comprising them are also useful in the manufacture of a medicament for therapeutic applications.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition provided herein the biological activity of a composition provided herein. According to particular embodiments, in view of the present disclosure, any pharmaceutically acceptable carrier suitable for use in an antibody pharmaceutical composition can be used herein.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers can be used in formulating the pharmaceutical compositions provided herein.

In one embodiment, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment, the pH of the formulation is from about 7.0 to about 9.5. In another embodiment, the pH of the formulation is from about 3.0 to about 7.0.

In another embodiment, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments.

In another embodiment, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments.

In another embodiment, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars can include mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethyl-cellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments.

In another embodiment, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments.

In another embodiment, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment, the pharmaceutical composition comprises a stabilizer, wherein said stabilizer is carboxy-/hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments.

In further embodiments, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments.

In a further embodiment, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments.

In another general aspect, provided herein is a method of producing a pharmaceutical composition comprising an antibody or antigen-binding fragment thereof provided herein, comprising combining an antibody or antigen-binding fragment thereof with a pharmaceutically acceptable carrier to obtain the pharmaceutical composition.

### Alternative Binding Agents

While TRGV9 antibodies are exemplified herein, it is understood that other molecules that bind to TRGV9 (TRGV9 molecules) are also contemplated. Such TRGV9 molecules include alternative binding agents, include equivalents of the antibodies provided herein. In some embodiments, the TRGV9 molecules of the present disclosure comprise a non-immunoglobulin binding agent. In some embodiments, the first binding domain comprises a non-immunoglobulin binding agent. In some embodiments, the second binding domain comprises a non-immunoglobulin binding agent.

In certain embodiments, such a non-immunoglobulin binding agent can bind the same targets exemplified herein. For example, in som embodiment, the non-immunoglobuling bindin gagent can bind to the same epitope as an antibody disclosed herein. In some embodiments, a non-immunoglobulin binding agent is identified as an agent that displaces or is displaced by an antibody of the present disclosure in a competitive binding assay. These alternative binding agents may include, for example, any of the engineered protein scaffolds known in the art. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352: 95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac7d from Sulfolobus acidolarius (see, e.g., Krehenbrink et al., 2008, J. Mol. Biol. 383:1058-68); affilins, based on human y-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

### Methods of Use

The functional activity of antibodies provided herein can be characterized by methods known in the art and as described herein. Methods for characterizing antibodies and antigen-binding fragments thereof include, but are not limited to, affinity and specificity assays including Biacore, ELISA, and OCTETRED analysis; binding assays to detect the binding of antibodies to target cells by FACS; binding assays to detect the binding of antibodies to the target antigen on cells. According to particular embodiments, the methods for characterizing antibodies and antigen-binding fragments thereof include those described below. In certain embodiments, the antibody is a TRGV9 antibody provided herein. In certain embodiments, the antibody is a second binding domain that binds to a second target provided herein. In some embodiments, the antibody is a multispecific TRGV9 antibody provided herein. According to particular embodiments, the methods for characterizing antibodies and antigen-binding fragments thereof that bind TRGV9 and/or another target antigen include those described below.

In one general aspect, provided is a method of activating a T cell expressing TRGV9, comprising contacting the T cell with a TRGV9 antibody provided herein. In some embodiments, the antibody is a multispecific TRGV9 antibody. In some embodiments, the contacting results in an increase in CD69, CD25, and/or Granzyme B expression, as compared to a control T cell expressing TRGV9. In certain embodiments, the T cell is a γδ T cell.

In another general aspect, provided is a method of inactivating a T cell expressing TRGV9, comprising contacting the T cell with a TRGV9 antibody provided herein. In some embodiments, the antibody is a multispecific TRGV9 antibody. In certain embodiments, the T cell is a γδ T cell.

In another general aspect, provided is a method of blocking activation of a T cell expressing TRGV9, comprising contacting the T cell with a TRGV9 antibody provided herein. In some embodiments, the antibody is a multispecific TRGV9 antibody. In certain embodiments, the T cell is a γδ T cell.

In another general aspect, provided is a method of modulating the activation of a T cell expressing TRGV9, comprising contacting the T cell with a TRGV9 antibody provided herein. In some embodiments, the antibody is a multispecific TRGV9 antibody. In certain embodiments, the T cell is a γδ T cell.

In another aspect, provided herein is a method of directing a T cell expressing TRGV9 to a target cell, the method comprising contacting the T cell with a multispecific TRGV9 antibody provided herein. In some embodiments, the contacting directs the T cell to the target cell.

Also provided is a method of targeting an antigen on the surface of a target cell, the method comprising exposing the target cell to a multispecific TRGV9 antibody provided herein.

Also provided is a method of targeting an antigen on the surface of a target cell, the method comprising exposing the target cell to a pharmaceutical composition comprising a multispecific TRGV9 antibody provided herein.

In another aspect provided is a method of directing a T cell expressing TRGV9 to a target cell, the method comprising contacting the T cell with a multispecific TRGV9 antibody provided herein, wherein the contacting directs the T cell to the target cell. In another aspect provided is a method of inhibiting growth or proliferation of a target cell, the method comprising contacting the target cell with a multispecific TRGV9 antibody provided herein, wherein the contacting inhibits growth or proliferation of the target cell. In some embodiments, the target cell is in the presence of the T cell expressing TRGV9 while in contact with the multispecific TRGV9 antibody. In some embodiments, the target cell expresses a second target that is not TRGV9. In some embodiments, the T cell is a γδ T cell. In some embodiments, the second target is an antigen of the second target. In some embodiments, the second target is an epitope of the second target. In some embodiments, the second target is on the surface of the target cell. In some embodiments, the second target is CD123. In some embodiments, the second target is CD33. In some embodiments, the second target is TRBC1. In some embodiments, the second target is BCMA. In some embodiments, the second target is PSMA. In one embodiment, the target cell is a cancer cell.

Also provided is a method for inhibiting growth or proliferation of target cells. In another aspect, provided is a TRGV9 antibody provided herein for use in inhibiting the growth or proliferation of target cells. The method can comprise contacting the target cells with a multispecific TRGV9 antibody provided herein, wherein the contacting inhibits the growth or proliferation of the target cells. In some embodiments, the target cells are in the presence of a T cell expressing TRGV9 while in contact with the multispecific antibody. In a specific embodiment, the T cell is a γδ T cell. In another aspect provided is a method for eliminating target cells in a subject, comprising administering an effective amount of a multispecific TRGV9 antibody provided herein to the subject. In another aspect, provided is a multispecific TRGV9 antibody provided herein for use in the elimination of target cells in a subject. In another aspect, provided is a method for treating a disease, disorder or condition (hereafter “disease”) caused all or in part by a target cell in a subject, comprising administering an effective amount of a multispecific TRGV9 antibody provided herein to the subject. In another aspect, provided is a multispecific TRGV9 antibody provided herein for use in the treatment of a disease caused all or in part by a target cell in a subject. In another aspect, provided is a method for preventing a disease caused all or in part by a target cell in a subject, comprising administering an effective amount of a multispecific TRGV9 antibody provided herein to the subject. In another aspect, provided is a multispecific TRGV9 antibody provided herein for use in the prevention of a disease caused all or in part by a target cell in a subject. In another aspect, provided is a method for modulating a disease caused all or in part by a target cell in a subject, comprising administering an effective amount of a multispecific TRGV9 antibody provided herein to the subject. In another aspect, provided is a multispecific TRGV9 antibody provided herein for use in the modulation of a disease caused all or in part by a target cell in a subject. In another aspect, provided is a TRGV9 antibody provided herein for use in a medicine. In some embodiments, the target cell expresses a second target that is not TRGV9. In some embodiments, the second target is on the surface of the target cell. In some embodiments, the second target is CD123. In some embodiments, the second target is CD33. In some embodiments, the second target is TRBC1. In some embodiments, the second target is BCMA. In one embodiment, the target cell is a cancer cell. In one embodiment, the target cell is a T cell. In one embodiment, the target cell is a B cell. In one embodiment, the target cell is a dendritic cell. In one embodiment, the target cell is a NK cell. In one embodiment, the target cell is a stem cell. In one embodiment, the target cell is a stem cell precursor. In one embodiment, the target cell is a monocyte. In one embodiment, the target cell is a macrophage. In one embodiment, the target cell is a granulocyte. In one embodiment, the target cell is a platelet. In one embodiment, the target cell is an erythrocyte. In one embodiment, the target cell is an endothelial cell. In one embodiment, the target cell is an epithelial cell. In one embodiment, the second target is a pathogen. In one embodiment, the target cell is a cell comprising a pathogen. In one embodiment, the target cell is a blood cell. In one embodiment, the target cell is a myeloid cell. In some embodiments, the subject is a subject in need thereof. In some embodiments, the subject is a human. In certain embodiments, the method further comprises identifying a subject in need thereof.

In one embodiment of the various methods provided herein, the target cell is a cancer cell, and the second binding domain of the multispecific TRGV9 antibody binds a cancer antigen. In one embodiment of the various methods provided herein, the target cell is a T cell, and the second binding domain of the multispecific TRGV9 antibody binds a T cell antigen. In one embodiment of the various methods provided herein, the target cell is a B cell, and the second binding domain of the multispecific TRGV9 antibody binds a B cell antigen. In one embodiment of the various methods provided herein, the target cell is a dendritic cell, and the second binding domain of the multispecific TRGV9 antibody binds a dendritic cell antigen. In one embodiment of the various methods provided herein, the target cell is a NK cell, and the second binding domain of the multispecific TRGV9 antibody binds a NK cell antigen. In one embodiment of the various methods provided herein, the target cell is a stem cell, and the second binding domain of the multispecific TRGV9 antibody binds a stem cell antigen. In one embodiment of the various methods provided herein, the target cell is a stem cell precursor, and the second binding domain of the multispecific TRGV9 antibody binds a stem cell precursor antigen. In one embodiment of the various methods provided herein, the target cell is a monocyte, and the second binding domain of the multispecific TRGV9 antibody binds a monocyte antigen. In one embodiment of the various methods provided herein, the target cell is a macrophage, and the second binding domain of the multispecific TRGV9 antibody binds a macrophage antigen. In one embodiment of the various methods provided herein, the target cell is a granulocyte, and the second binding domain of the multispecific TRGV9 antibody binds a granulocyte antigen. In one embodiment of the various methods provided herein, the target cell is a platelet, and the second binding domain of the multispecific TRGV9 antibody binds a platelet antigen. In one embodiment of the various methods provided herein, the target cell is an erythrocyte, and the second binding domain of the multispecific TRGV9 antibody binds an erythrocyte antigen. In one embodiment of the various methods provided herein, the target cell is an endothelial cell, and the second binding domain of the multispecific TRGV9 antibody binds an endothelial antigen. In one embodiment of the various methods provided herein, the target cell is an epithelial cell, and the second binding domain of the multispecific TRGV9 antibody binds an epithelial antigen. In one embodiment of the various methods provided herein, the second target is a pathogen, and the second binding domain of the multispecific TRGV9 antibody binds a pathogen antigen. In one embodiment of the various methods provided herein, the target cell is a cell comprising a pathogen, and the second binding domain of the multispecific TRGV9 antibody binds a pathogen antigen.

In a specific embodiment, the disease is a cancer. In some embodiments, the cancer is a cancer expressing the second target. In some embodiments, the cancer is a CD123-expressing cancer. In some embodiments, the cancer is a CD33-expressing cancer. In some embodiments, the cancer is a TRBC1-expressing cancer. In some embodiments, the cancer is a BCMA-expressing cancer. In some embodiments, the cancer is a PSMA-expressing cancer.

The cancer can, for example, be a hematologic cancer. The hematologic cancer can, for example, be a leukemia, a lymphoma, and a myeloma. The leukemia can be an acute myeloid leukemia (AML) or an acute lymphocytic leukemia (ALL).

According to particular embodiments, provided are compositions used in the treatment of a cancer. For cancer therapy, the compositions can be used in combination with another treatment including, but not limited to, a chemotherapy, an anti-CD20 mAb, an anti-TIM-3 mAb, an anti-CTLA-4 antibody, an anti-PD-L1 antibody, an anti-PD-1 antibody, a PD-1/PD-L1 therapy, IDO, an anti-OX40 antibody, an anti-GITR antibody, an anti-CD40 antibody, an anti-CD38 antibody, cytokines, oncolytic viruses, TLR agonists, STING agonist, other immuno-oncology drugs, an antiangiogenic agent, a radiation therapy, an antibody-drug conjugate (ADC), a targeted therapy, or other anticancer drugs.

According to embodiments of the invention, the pharmaceutical composition comprises an effective amount of a TRGV9 antibody provided herein. In a specific embodiment the TRGV9 antibody is a multispecific TRGV9 antibody.

As used herein, the term “effective amount” refers to an amount of an active ingredient or component that elicits the desired biological or medicinal response in a subject.

According to particular embodiments, an effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

In some embodiments, a TRGV9 bispecific antibody provided herein is used in combination with a supplemental therapy.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

TRGV9 antibodies provided herein may also be used as agents to detect cells expressing TRGV9. Thus, in another aspect, provided is a method of detecting a cell expressing TRGV9, comprising contacting a cell with a TRGV9 antibody provided herein. In certain embodiments, the cell is in a population of cells. In certain embodiments, the detecting is by ELISA. In some embodiments, the detecting is by FACS analysis. Also provided are kits comprising a TRGV9 antibody provided herein, and instructions for use.

### Enrichment and Detection Methods

In one aspect, the TRGV9 antibodies provided herein are used as agents to detect TRGV9-expressing cells. Thus, in other methods, provided is a method of detecting a cell expressing TRGV9, comprising contacting a cell with a TRGV9 antibody provided herein. In certain embodiments, the detecting is by ELISA. In some embodiments, the detecting is by FACS analysis. Also provided are kits comprising a TRGV9 antibody provided herein, and instructions for use.

Enrichment, isolation, separation, purification, sorting, selecting, capturing or detecting, or any combination thereof can be done using known technologies such as bead, microfluidics, solid support, columns, and the like. For example, TRGV9 cells may be separated or visualized using known methods when bound to the TRGV9 antibodies provided herein.

The TRGV9 antibodies or multispecific TRGV9 antibodies provided herein can be used to selectively enrich, isolate, separate, purify, sort, select, capture or detect TRGV9-expressing cells. The TRGV9 antibodies or multispecific TRGV9 antibodies provided herein may be utilized in a bispecific format, e.g. containing a first antigen binding domain that specifically binds TRGV9 and a second antigen binding domain that specifically binds a second target. In other embodiments, the multispecific TRGV9 antibodies provided herein may be utilized in a format that further incorporates a third antigen binding domain that specifically binds a third antigen (e.g., at a trispecific antibody). In other embodiments, the multispecific TRGV9 antibodies provided herein may be utilized in a format that further incorporates a fourth antigen binding domain that specifically binds a fourth antigen. (e.g., as a quadraspecific antibody).

In one aspect, provided herein is a method of enriching a TRGV9-expressing cell comprising: providing a sample comprising the TRGV9-expressing cell; contacting the sample with a TRGV9 antibody provided herein; and enriching the TRGV9-expressing cell bound to the TRGV9 antibody. In one aspect, provided herein is a method of isolating a TRGV9-expressing cell comprising: providing a sample comprising the TRGV9-expressing cell; contacting the sample with a TRGV9 antibody provided herein; and isolating the TRGV9-expressing cell bound to the TRGV9 antibody. In one aspect, provided herein is a method of separating a TRGV9-expressing cell comprising: providing a sample comprising the TRGV9-expressing cell; contacting the sample with a TRGV9 antibody provided herein; and separating the TRGV9-expressing cell bound to the TRGV9 antibody. In one aspect, provided herein is a method of purifying a TRGV9-expressing cell comprising: providing a sample comprising the TRGV9-expressing cell; contacting the sample with a TRGV9 antibody provided herein; and purifying the TRGV9-expressing cell bound to the TRGV9 antibody. In one aspect, provided herein is a method of sorting a TRGV9-expressing cell comprising: providing a sample comprising the TRGV9-expressing cell; contacting the sample with a TRGV9 antibody provided herein; and sorting the TRGV9-expressing cell bound to the TRGV9 antibody. In one aspect, provided herein is a method of selecting a TRGV9-expressing cell comprising: providing a sample comprising the TRGV9-expressing cell; contacting the sample with a TRGV9 antibody provided herein; and selecting the TRGV9-expressing cell bound to the TRGV9 antibody. In one aspect, provided herein is a method of capturing a TRGV9-expressing cell comprising: providing a sample comprising the TRGV9-expressing cell; contacting the sample with a TRGV9 antibody provided herein; and capturing the TRGV9-expressing cell bound to the TRGV9 antibody. In one aspect, provided herein is a method of detecting a TRGV9-expressing cell comprising: providing a sample comprising the TRGV9-expressing cell; contacting the sample with a TRGV9 antibody provided herein; and detecting the TRGV9-expressing cell bound to the TRGV9 antibody.

In one aspect, provided herein is a method of enriching a TRGV9-expressing cell comprising: contacting a TRGV9-expressing cell with a TRGV9 antibody provided herein; and enriching the TRGV9-expressing cell bound to the TRGV9 antibody. In one aspect, provided herein is a method of isolating a TRGV9-expressing cell comprising: contacting a TRGV9-expressing cell with a TRGV9 antibody provided herein; and isolating the TRGV9-expressing cell bound to the TRGV9 antibody. In one aspect, provided herein is a method of separating a TRGV9-expressing cell comprising: contacting a TRGV9-expressing cell with a TRGV9 antibody provided herein; and separating the TRGV9-expressing cell bound to the TRGV9 antibody. In one aspect, provided herein is a method of purifying a TRGV9-expressing cell comprising: contacting a TRGV9-expressing cell with a TRGV9 antibody provided herein; and purifying the TRGV9-expressing cell bound to the TRGV9 antibody. In one aspect, provided herein is a method of sorting a TRGV9-expressing cell comprising: contacting a TRGV9-expressing cell with a TRGV9 antibody provided herein; and sorting the TRGV9-expressing cell bound to the TRGV9 antibody. In one aspect, provided herein is a method of selecting a TRGV9-expressing cell comprising: contacting a TRGV9-expressing cell with a TRGV9 antibody provided herein; and selecting the TRGV9-expressing cell bound to the TRGV9 antibody. In one aspect, provided herein is a method of capturing a TRGV9-expressing cell comprising: contacting a TRGV9-expressing cell with a TRGV9 antibody provided herein; and capturing the TRGV9-expressing cell bound to the TRGV9 antibody. In one aspect, provided herein is a method of detecting a TRGV9-expressing cell comprising: contacting a TRGV9-expressing cell with a TRGV9 antibody provided herein; and detecting the TRGV9-expressing cell bound to the TRGV9 antibody.

In one aspect, provided herein is a method of enriching a TRGV9-expressing cell comprising: contacting a TRGV9-expressing cell with a TRGV9 antibody provided herein; and enriching the TRGV9-expressing cell based on binding of the TRGV9-expressing cell to the TRGV9 antibody. In one aspect, provided herein is a method of isolating a TRGV9-expressing cell comprising: contacting a TRGV9-expressing cell with a TRGV9 antibody provided herein; and isolating the TRGV9-expressing cell based on binding of the TRGV9-expressing cell to the TRGV9 antibody. In one aspect, provided herein is a method of separating a TRGV9-expressing cell comprising: contacting a TRGV9-expressing cell with a TRGV9 antibody provided herein; and separating the TRGV9-expressing cell based on binding of the TRGV9-expressing cell to the TRGV9 antibody. In one aspect, provided herein is a method of purifying a TRGV9-expressing cell comprising: contacting a TRGV9-expressing cell with a TRGV9 antibody provided herein; and purifying the TRGV9-expressing cell based on binding of the TRGV9-expressing cell to the TRGV9 antibody. In one aspect, provided herein is a method of sorting a TRGV9-expressing cell comprising: contacting a TRGV9-expressing cell with a TRGV9 antibody provided herein; and sorting the TRGV9-expressing cell based on binding of the TRGV9-expressing cell to the TRGV9 antibody. In one aspect, provided herein is a method of selecting a TRGV9-expressing cell comprising: contacting a TRGV9-expressing cell with a TRGV9 antibody provided herein; and selecting the TRGV9-expressing cell based on binding of the TRGV9-expressing cell to the TRGV9 antibody. In one aspect, provided herein is a method of capturing a TRGV9-expressing cell comprising: contacting a TRGV9-expressing cell with a TRGV9 antibody provided herein; and capturing the TRGV9-expressing cell based on binding of the TRGV9-expressing cell to the TRGV9 antibody. In one aspect, provided herein is a method of detecting a TRGV9-expressing cell comprising: contacting a TRGV9-expressing cell with a TRGV9 antibody provided herein; and detecting the TRGV9-expressing cell based on binding of the TRGV9-expressing cell to the TRGV9 antibody.

In certain embodiments of the methods, the TRGV9-expressing cell is a T cell. In some embodiments of the methods, the TRGV9-expressing cell is in a population of cells. In some embodiments of the methods, the TRGV9-expressing cell is in a population of lymphocytes. In some embodiments of the methods, the TRGV9-expressing cell is in a population of T cells. In some embodiments of the methods, the TRGV9-expressing cell is provided as a population of cells. In some embodiments of the methods, the TRGV9-expressing cell is provided as a population of lymphocytes. In some embodiments of the methods, the TRGV9-expressing cell is provided as a population of T cells. In some embodiments of the methods, the TRGV9-expressing cell is provided as a sample comprising a population of cells. In some embodiments of the methods, the TRGV9-expressing cell is provided as a sample comprising a population of lymphocytes. In some embodiments of the methods, the TRGV9-expressing cell is provided as a sample comprising a population of T cells. In some embodiments of the methods, the sample is a blood sample. In some embodiments of the methods, the sample is a tissue sample. In some embodiments of the methods, the sample is a tissue culture sample.

In some embodiments of the methods, the TRGV9 antibody is a multispecific TRGV9 antibody provided herein. In some embodiments of the methods, the TRGV9 antibody is a bispecific TRGV9 antibody provided herein. In some embodiments of the methods, the TRGV9 antibody is a trispecific TRGV9 antibody provided herein. In some embodiments of the methods, the TRGV9 antibody is a quadraspecific TRGV9 antibody provided herein. In certain embodiments, the TRGV9 antibody specifically binds to TRGV9. In one embodiment, the multispecific TRGV9 antibody comprises: (a) a first binding domain that binds TRGV9, and (b) a second binding domain that binds to a second target. In one embodiment, the multispecific TRGV9 antibody comprises: (a) a first binding domain that binds TRGV9, and (b) a second binding domain that binds to a second target, and (c) a third binding domain that binds to a third target. In one embodiment, the multispecific TRGV9 antibody comprises: (a) a first binding domain that binds TRGV9, and (b) a second binding domain that binds to a second target, (c) a third binding domain that binds to a third target, and (d) a fourth binding domain that binds to a fourth target. In one embodiment, the multispecific TRGV9 antibody comprises: (a) a first binding domain that specifically binds TRGV9, and (b) a second binding domain that specifically binds to a second target. In one embodiment, the multispecific TRGV9 antibody comprises: (a) a first binding domain that specifically binds TRGV9, and (b) a second binding domain that specifically binds to a second target, and (c) a third binding domain that specifically binds to a third target. In one embodiment, the multispecific TRGV9 antibody comprises: (a) a first binding domain that specifically binds TRGV9, and (b) a second binding domain that specifically binds to a second target, (c) a third binding domain that specifically binds to a third target, and (d) a fourth binding domain that specifically binds to a fourth target.

In some embodiments of the methods, the TRGV9 antibody is a multispecific TRGV9 antibody, wherein the second target is CD123. In some embodiments of the methods, the TRGV9 antibody is a multispecific TRGV9 antibody, wherein the second target is CD33. In some embodiments of the methods, the TRGV9 antibody is a multispecific TRGV9 antibody, wherein the second target is TRBC1. In some embodiments of the methods, the TRGV9 antibody is a multispecific TRGV9 antibody, wherein the second target is BCMA. In some embodiments of the methods, the TRGV9 antibody is a multispecific TRGV9 antibody, wherein the second target is PSMA.

In specific embodiments of the methods provided herein, the method uses multi-marker detection. In some embodiments, the multi-marker detection uses a multispecific TRGV9 antibody provided herein. In some embodiments, the multi-marker detection uses a bispecific TRGV9 antibody provided herein. In some embodiments, the multi-marker detection uses a trispecific TRGV9 antibody provided herein. In some embodiments, the multi-marker detection uses a quadraspecific TRGV9 antibody provided herein.

In certain embodiments of the methods provided herein, the methods are included as steps in a T cell manufacturing process. In certain embodiments, the cells are CAR-T cells. In certain embodiments of the methods provided herein, the methods are included as steps in a T cell modification process.

In certain embodiments of the methods provided herein, the methods are included as steps in a diagnostic method. In certain embodiments of the methods provided herein, the methods are included as steps in a method to quantify the TRGV9-expressing T cells.

In certain embodiments of the methods provided herein, the method further comprises expanding the enriched, isolated, separated, purified, sorted, selected, captured or detected TRGV9-expressing cells. In certain embodiments, the expanding is in vitro. In certain embodiments, the expanding is in vivo. In certain embodiments of the methods provided herein, the method further comprises growing the enriched, isolated, separated, purified, sorted, selected, captured or detected TRGV9-expressing cells. In certain embodiments, the growing is in vitro. In certain embodiments, the growing is in vivo. In certain embodiments of the methods provided herein, the method further comprises quantifying the enriched, isolated, separated, purified, sorted, selected, captured or detected TRGV9-expressing cells.

### EMBODIMENTS

This invention provides the following non-limiting embodiments.

In one set of embodiments, provided are:
1. A bispecific antibody comprising:
   (a) a first binding domain that binds to a TRGV9 antigen, and
   (b) a second binding domain that binds to an antigen on the surface of a cancer cell.
2. The bispecific antibody of embodiment 1, wherein the first binding domain comprises:
   (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and
   (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6.
3. The bispecific antibody of embodiment 2, wherein the first binding domain comprises a VH having an amino acid sequence of SEQ ID NO:7.
4. The bispecific antibody of embodiment 2, wherein the first binding domain comprises a VL having an amino acid sequence of SEQ ID NO:8.
5. The bispecific antibody of embodiment 2, wherein the first binding domain comprises a VH having an amino acid sequence of SEQ ID NO:7, and a VL having an amino acid sequence of SEQ ID NO:8.
6. The bispecific antibody of embodiment 1, wherein the first binding domain comprises:
   (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:31; and
   (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6.
7. The bispecific antibody of embodiment 6, wherein the first binding domain comprises a VH having an amino acid sequence of SEQ ID NO:34.
8. The bispecific antibody of embodiment 6, wherein the first binding domain comprises a VL having an amino acid sequence of SEQ ID NO:8.
9. The bispecific antibody of embodiment 6, wherein the first binding domain comprises a VH having an amino acid sequence of SEQ ID NO:34, and a VL having an amino acid sequence of SEQ ID NO:8.
10. The bispecific antibody of embodiment 1, wherein the first binding domain comprises:
   (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:32; and
   (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6.
11. The bispecific antibody of embodiment 10, wherein the first binding domain comprises a VH having an amino acid sequence of SEQ ID NO:35.
12. The bispecific antibody of embodiment 10, wherein the first binding domain comprises a VL having an amino acid sequence of SEQ ID NO:8.
13. The bispecific antibody of embodiment 10, wherein the first binding domain comprises a VH having an amino acid sequence of SEQ ID NO:35, and a VL having an amino acid sequence of SEQ ID NO:8.
14. The bispecific antibody of embodiment 1, wherein the first binding domain comprises:
   (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:33; and
   (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6.
15. The bispecific antibody of embodiment 14, wherein the first binding domain comprises a VH having an amino acid sequence of SEQ ID NO:36.
16. The bispecific antibody of embodiment 14, wherein the first binding domain comprises a VL having an amino acid sequence of SEQ ID NO:8.
17. The bispecific antibody of embodiment 14, wherein the first binding domain comprises a VH having an amino acid sequence of SEQ ID NO:36, and a VL having an amino acid sequence of SEQ ID NO:8.
18. The bispecific antibody of any one of embodiments 1 to 17, wherein the antigen on the surface of the cancer cell is a tumor-specific antigen, a tumor associated antigen, or a neoantigen.
19. The bispecific antibody of any one of embodiments 1 to 18, wherein the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.
20. The bispecific antibody of any one of embodiments 1 to 19, wherein
   (i) the adrenal cancer is an adrenocortical carcinoma (ACC), adrenal cortex cancer, pheochromocytoma, or neuroblastoma;
   (ii) the anal cancer is a squamous cell carcinoma, cloacogenic carcinoma, adenocarcinoma, basal cell carcinoma, or melanoma;
   (iii) the appendix cancer is a neuroendocrine tumor (NET), mucinous adenocarcinoma, goblet cell carcinoid, intestinal-type adenocarcinoma, or signet-ring cell adenocarcinoma;
   (iv) the bile duct cancer is an extrahepatic bile duct cancer, adenocarcinomas, hilar bile duct cancer, perihilar bile duct cancer, distal bile duct cancer, or intrahepatic bile duct cancer;
   (v) the bladder cancer is transitional cell carcinoma (TCC), papillary carcinoma, flat carcinoma, squamous cell carcinoma, adenocarcinoma, small-cell carcinoma, or sarcoma;
   (vi) the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer;
   (vii) the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma;
   (viii) the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer;
   (ix) the cervical cancer is a squamous cell carcinoma, or adenocarcinoma;
   (x) the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma;
   (xi) the esophageal cancer is an adenocarcinoma or squamous cell carcinoma;
   (xii) the gall bladder cancer is an adenocarcinoma, papillary adenocarcinoma, adenosquamous carcinoma, squamous cell carcinoma, small cell carcinoma, or sarcoma;
   (xiii) the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT);
   (xiv) the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer;
   (xv) the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL);
   (xvi) the intestinal cancer is a small intestine cancer, small bowel cancer, adenocarcinoma, sarcoma, gastrointestinal stromal tumors, carcinoid tumors, or lymphoma;
   (xvii) the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma;
   (xviii) the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS);
   (xix) the liver cancer is a hepatocellular carcinoma (HCC), fibrolamellar HCC, cholangiocarcinoma, angiosarcoma, or liver metastasis;
   (xx) the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor;
   (xxi) the melanoma is a superficial spreading melanoma, nodular melanoma, acral-lentiginous melanoma, lentigo maligna melanoma, amelanotic melanoma, desmoplastic melanoma, ocular melanoma, or metastatic melanoma;
   (xxii) the mesothelioma is a pleural mesothelioma, peritoneal mesothelioma, pericardial mesothelioma, or testicular mesothelioma;
   (xxiii) the multiple myeloma is an active myeloma or smoldering myeloma;
   (xxiv) the neuroendocrine tumor, is a gastrointestinal neuroendocrine tumor, pancreatic neuroendocrine tumor, or lung neuroendocrine tumor;
   (xxv) the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma;
   (xxvi) the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer;
   (xxvii) the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst;
   (xxviii) the pancreatic cancer is a pancreatic exocrine gland cancer, pancreatic endocrine gland cancer, or pancreatic adenocarcinoma, islet cell tumor, or neuroendocrine tumor;
   (xxix) the prostate cancer is a prostate adenocarcinoma, prostate sarcoma, transitional cell carcinoma, small cell carcinoma, or neuroendocrine tumor;
   (xxx) the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer; (xxxi) the skin cancer is a basal cell carcinoma, squamous cell carcinoma, melanoma, Merkel cell carcinoma, Kaposi sarcoma (KS), actinic keratosis, skin lymphoma, or keratoacanthoma;
   (xxxii) the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma;
   (xxxiii) the spinal cancer is a spinal metastatic tumor;
   (xxxiv) the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid;
   (xxxv) the testicular cancer is a seminoma, non-seminoma, embryonal carcinoma, yolk sac carcinoma, choriocarcinoma, teratoma, gonadal stromal tumor, leydig cell tumor, or sertoli cell tumor;
   (xxxiv) the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer;
   (xxxv) the thyroid cancer is a papillary carcinoma, follicular carcinoma, Hürthle cell carcinoma, medullary thyroid carcinoma, or anaplastic carcinoma;
   (xxxvi) the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma;
   (xxxvii) the vaginal cancer is a squamous cell carcinoma, adenocarcinoma, melanoma, or sarcoma; or
   (xxxviii) the vulvar cancer is a squamous cell carcinoma or adenocarcinoma.
21. The bispecific antibody of any one of embodiments 1 to 20, wherein the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAP1, TARP, TROP2, VEGF, or VEGF-R.
22. The bispecific antibody of any one of embodiments 1 to 20, wherein the cancer antigen is a CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1 antigen.
23. The bispecific antibody of any one of embodiments 1 to 22, wherein the TRGV9 is present on the surface of a γδ T cell.
24. The bispecific antibody of any one of embodiments 1 to 22, wherein the TRGV9 is present on the surface of a γδ T cell, and the antigen expressed on the surface of the cancer cell is a cancer antigen.
25. The bispecific antibody of embodiment 24, wherein the cancer cell is killed when the bispecific antibody binds to the TRGV9 on the surface of the γδ T cell and the antigen on the surface of the cancer cell.
26. The bispecific antibody of any one of embodiments 1 to 25, wherein the first binding domain is humanized, the second binding domain is humanized, or both the first binding domain and the second binding domain are humanized.
27. The bispecific antibody of any one of embodiments 1 to 26, wherein the bispecific antibody is an IgG antibody.
28. The bispecific antibody of embodiment 27, wherein the IgG antibody is an IgG1, IgG2, IgG3, IgG4 antibody.
29. The bispecific antibody of any one of embodiments 24 to 28, wherein the bispecific antibody induces γδ T cell dependent cytotoxicity of the cancer cell in vitro with an EC₅₀ of less than about 500 pM.
30. The bispecific antibody of embodiment 29, wherein the bispecific antibody induces γδ T cell dependent cytotoxicity of the cancer cell in vitro with an EC₅₀ of less than about 300 pM.
31. The bispecific antibody of embodiment 30, wherein the bispecific antibody induces γδ T cell dependent cytotoxicity of the cancer cell in vitro with an EC₅₀ of less than about 160 pM.
32. The bispecific antibody of any one of embodiments 29 to 31, wherein the EC₅₀ is assessed with a mixture of γδ T effector cells and target cells expressing the cancer antigen.
33. The bispecific antibody of embodiment 32, wherein the effector cell to target cell ratio is about 0.01 to 1 to about 5 to 1.
34. The bispecific antibody of embodiment 33, wherein the effector cell to target cell ratio is about 0.1 to 1 to about 2 to 1.
35. The bispecific antibody of embodiment 34, wherein the effector cell to target cell ratio is about 1:1.
36. The bispecific antibody of any one of embodiments 1 to 35, wherein the bispecific antibody is multivalent.
37. The bispecific antibody of embodiment 36, wherein the bispecific antibody is capable of binding at least three antigens.
38. The bispecific antibody of embodiment 37, wherein the bispecific antibody is capable of binding at least five antigens.
39. A bispecific antibody comprising: a first means capable of binding TRGV9 on the surface of the γδ T cell; and a second means capable of binding a cancer antigen.
40. The bispecific antibody of embodiment 39, wherein the cancer antigen is on the surface of a cancer cell.
41. A nucleic acid encoding the bispecific antibody of any one of embodiments 1 to 40.
42. A vector comprising the nucleic acid of embodiment 41.
43. A host cell comprising the vector of embodiment 42.
44. A kit comprising the vector of embodiment 42 and packaging for the same.
45. A pharmaceutical composition comprising the bispecific antibody of any one of embodiments 1 to 40, and a pharmaceutically acceptable carrier.
46. A method of producing the pharmaceutical composition of embodiment 45, comprising combining the bispecific antibody with a pharmaceutically acceptable carrier to obtain the pharmaceutical composition.
47. A process for making an antibody that binds to more than one target molecule, the molecule comprising: a step for performing a function of obtaining a binding domain capable of binding to TRGV9 antigen on a γδ T cell; a step for performing a function of obtaining a binding domain capable of binding to an antigen on the surface of a cancer cell; and a step for performing a function of providing an antibody capable of binding to a TRGV9 antigen on a γδ T cell and an antigen on the surface of a cancer cell.
48. The process of embodiment 47, wherein the step for performing a function of obtaining a binding domain capable of binding to an antigen on the surface of a cancer cell is repeated n times and further comprising n steps for performing a function of providing a binding domain capable of binding to a TRGV9 antigen on a γδ T cell and n number of target molecules, wherein n is at least 2.
49. A method of directing a γδ T cell expressing TRGV9 to a cancer cell, the method comprising contacting the γδ T cell with the bispecific antibody of any one of embodiments 1 to 40, wherein the contacting directs the γδ T cell to the cancer cell.
50. A method of inhibiting growth or proliferation of cancer cells expressing a cancer antigen on the cell surface, the method comprising contacting the cancer cells with the bispecific antibody of any one of embodiments 1 to 40, wherein contacting the cancer cells with the pharmaceutical composition inhibits growth or proliferation of the cancer cells.
51. The method of embodiment 50, wherein the cancer cells are in the presence of a γδ T cell expressing TRGV9 while in contact with the bispecific antibody.
52. A method for eliminating cancer cells or treating cancer in a subject, comprising administering an effective amount of the bispecific antibody of any one of embodiments 1 to 40 to the subject.
53. The method of embodiment 52, wherein the subject is a subject in need thereof.
54. The method of embodiments 52 or 53, wherein the subject is a human.
55. A method of activating a γδ T cell expressing TRGV9, comprising contacting the γδ T cell with the bispecific antibody of any one of embodiments 1 to 40.
56. The method of embodiment 55, wherein the contacting results in an increase in CD69, CD25, and/or Granzyme B expression, as compared to a control γδ T cell expressing TRGV9.
57. The method of embodiment 50 or 51, wherein
   (i) the cancer cells are cells of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer;
   (ii) the cancer antigen is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAP1, TARP, TROP2, VEGF, or VEGF-R; and/or
   (iii) the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, or BRCA1
58. The method of any one of embodiments 52 to 56, wherein the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.
59. An isolated TRGV9 bispecific antibody or antigen binding fragment thereof, the isolated TRGV9 bispecific antibody or antigen binding fragment thereof comprising:
   a. a first heavy chain (HC1);
   b. a second heavy chain (HC2);
   c. a first light chain (LC1); and
   d. a second light chain (LC2),
   wherein HC1 is associated with LC1 and HC2 is associated with LC2, and
   wherein HC1 comprises a heavy chain complementarity determining region 1 (HCDR1), HCDR2, and HCDR3 comprising the amino acid sequences of:
   i. SEQ ID NO:1, SEQ ID NO:2, and SEQ ID NO:3, respectively,
   ii. SEQ ID NO:1, SEQ ID NO:2, and SEQ ID NO:31, respectively,
   iii. SEQ ID NO:1, SEQ ID NO:2, and SEQ ID NO:32, respectively, or
   iv. SEQ ID NO:1, SEQ ID NO:2, and SEQ ID NO:33, respectively,
   and LC1 comprises a light chain complementarity determining region 1 (LCDR1), LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen.
60. The isolated TRGV9 bispecific antibody or antigen binding fragment thereof embodiment 59, wherein HC1 comprises an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8.
61. The isolated TRGV9 bispecific antibody or antigen binding fragment thereof embodiment 60, wherein HC1 comprises the amino acid sequence selected from SEQ ID NO:7, SEQ ID NO:34, SEQ ID NO:35, or SEQ ID NO:36, and LC1 comprises the amino acid sequence of SEQ ID NO:8.
62. The isolated TRGV9 bispecific antibody or antigen binding fragment thereof any one of embodiments 59 to 61, wherein the binding site for a first antigen binds to TRGV9 on a γδ T cell.
63. The isolated TRGV9 bispecific antibody or antigen binding fragment thereof any one of embodiments 59 to 62, wherein the binding site for a second antigen binds to a cancer antigen present on the surface of a cancer cell.
64. The isolated TRGV9 bispecific antibody or antigen binding fragment of embodiment 63, wherein the binding of the bispecific antibody to TRGV9 present on the surface of the γδ T cell and the binding of the cancer antigen present on the surface of the cancer cell results in the killing of the cancer cell.
65. The isolated TRGV9 bispecific antibody or antigen binding fragment of any one of embodiments 59 to 64, wherein HC1 and LC1 are humanized.
66. The isolated TRGV9 bispecific antibody or antigen binding fragment thereof any one of embodiments 59 to 65, wherein HC2 and LC2 bind to CD123.
67. The isolated TRGV9 bispecific antibody or antigen binding fragment thereof any one of embodiments 59 to 66, wherein the bispecific antibody or antigen binding fragment thereof is an IgG1, an IgG2, an IgG3, or an IgG4 isotype.
68. The isolated TRGV9 bispecific antibody or antigen binding fragment thereof any one of embodiments 59 to 67, wherein the bispecific antibody or antigen binding fragment thereof is an IgG4 isotype.
69. The isolated TRGV9 bispecific antibody or antigen binding fragment thereof any one of embodiments 59 to 68, wherein the bispecific antibody or antigen binding fragment thereof induces γδ T cell dependent cytotoxicity of a cancer cell in vitro with an EC₅₀ of less than about 500 pM.
70. The isolated TRGV9 bispecific antibody or antigen binding fragment thereof embodiment 11, wherein the bispecific antibody or antigen binding fragment thereof induces γδ T cell dependent cytotoxicity of a cancer cell in vitro with an EC₅₀ of less than about 300 pM.
71. The isolated TRGV9 bispecific antibody or antigen binding fragment thereof embodiment 69, wherein the bispecific antibody or antigen binding fragment thereof induces γδ T cell dependent cytotoxicity of a cancer cell in vitro with an EC₅₀ of less than about 160 pM.
72. The isolated TRGV9 bispecific antibody or antigen binding fragment thereof any one of embodiments 69 to 71, wherein the EC₅₀ is assessed with a mixture of γδ T effector cells and Kasumi3 AML target cells.
73. The isolated TRGV9 bispecific antibody or antigen binding fragment thereof embodiment 72, wherein the effector cell to target cell ratio is about 0.01 to 1 to about 5 to 1.
74. The isolated TRGV9 bispecific antibody or antigen binding fragment thereof embodiment 73, wherein the effector cell to target cell ratio is about 0.1 to 1 to about 2 to 1.
75. The isolated TRGV9 bispecific antibody or antigen binding fragment thereof embodiment 74, wherein the effector cell to target cell ratio is about 1:1.
76. The isolated TRGV9 bispecific antibody or antigen binding fragment thereof any one of embodiments 59 to 75, wherein the bispecific antibody or antigen binding fragment thereof is multivalent.
77. The isolated TRGV9 bispecific antibody or antigen binding fragment thereof embodiment 76, wherein the bispecific antibody or antigen binding fragment thereof is capable of binding at least three antigens.
78. The isolated TRGV9 bispecific antibody or antigen binding fragment thereof embodiment 76, wherein the bispecific antibody or antigen binding fragment thereof is capable of binding at least five antigens.
79. An isolated γδ T cell bispecific antibody or antigen binding fragment thereof, the isolated γδ T cell bispecific antibody or antigen binding fragment thereof comprising:
   a. a HC1;
   b. a HC2;
   c. a LC1; and
   d. a LC2,
   wherein HC1 is associated with LC1 and HC2 is associated with LC2,
   wherein HC1 and LC1 form a binding site for a first antigen on a γδ T cell, and
   wherein HC2 and LC2 form a binding site for a second antigen.
80. A bispecific antibody comprising: a first means capable of specifically binding a T cell receptor gamma chain; and a second means capable of specifically binding a target molecule that is not a T cell receptor gamma chain.
81. A process for making a molecule capable of specifically binding to more than one target molecule, the molecule comprising: a step for performing a function of obtaining an oligopeptide or polypeptide capable of binding to a T cell receptor gamma chain; a step for performing a function of obtaining an oligopeptide or polypeptide capable of binding to a target; and a step for performing a function of providing a molecule capable of specifically binding to a T cell receptor gamma chain and a target molecule.
82. The process of embodiment 81, wherein the step for performing a function of obtaining an oligopeptide or polypeptide capable of binding to a target is repeated n times and further comprising n steps for performing a function of providing a molecule capable of specifically binding to a T cell receptor gamma chain and n number of target molecules, wherein n is at least 2.
83. An isolated anti-TRGV9/anti-CD123 bispecific antibody or antigen binding fragment thereof comprising:
   a. a HC1;
   b. a HC2
   c. a LC1; and
   d. a LC2,
   wherein HC1 is associated with LC1 and HC2 is associated with LC2, and
   wherein HC1 comprises a HCDR1, HCDR2, and HCDR3 comprising the amino acid sequences of:
   i. SEQ ID NO:1, SEQ ID NO:2, and SEQ ID NO:3, respectively,
   ii. SEQ ID NO:1, SEQ ID NO:2, and SEQ ID NO:31, respectively,
   iii. SEQ ID NO:1, SEQ ID NO:2, and SEQ ID NO:32, respectively, or
   iv. SEQ ID NO:1, SEQ ID NO:2, and SEQ ID NO:33, respectively,
   and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123.
84. The isolated anti-TRGV9/anti-CD123 bispecific antibody or antigen binding fragment thereof embodiment 83, wherein HC1 comprises an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8.
85. The isolated anti-TRGV9/anti-CD123 bispecific antibody or antigen binding fragment thereof embodiment 84, wherein HC1 comprises the amino acid sequence selected from SEQ ID NO:7, SEQ ID NO:34, SEQ ID NO:35, or SEQ ID NO:36, and LC1 comprises the amino acid sequence of SEQ ID NO:8.
86. The isolated anti-TRGV9/anti-CD123 bispecific antibody or antigen binding fragment thereof any one of embodiments 83 to 85, wherein HC2 comprises an amino acid sequence having at least 95% identity to the amino acid sequence of SEQ ID NO:15 and LC2 comprises an amino acid sequence having at least 95% identity to the amino acid sequence of SEQ ID NO:16.
87. The isolated anti-TRGV9/anti-CD123 bispecific antibody or antigen binding fragment thereof embodiment 86, wherein HC2 comprises the amino acid sequence of SEQ ID NO:15 and LC2 comprises the amino acid sequence of SEQ ID NO:16.
88. The isolated anti-TRGV9/anti-CD123 bispecific antibody or antigen binding fragment thereof any one of embodiments 83 to 87, wherein the TRGV9 is on the surface of a γδ T cell.
89. The isolated anti-TRGV9/anti-CD123 bispecific antibody or antigen binding fragment thereof any one of embodiments 85 to 88, wherein the CD123 is on the surface of a tumor cell or a CD34+ stem cell.
90. The isolated anti-TRGV9/anti-CD123 bispecific antibody or antigen binding fragment thereof any one of embodiments 83 to 89, wherein the binding of the bispecific antibody to TRGV9 present on the surface of the γδ T cell and the binding of the CD123 on the surface of the cancer cell results in the killing of the cancer cell.
91. The isolated anti-TRGV9/anti-CD123 bispecific antibody or antigen binding fragment thereof any one of embodiments 83 to 90, wherein HC1 and LC1 are humanized.
92. The isolated anti-TRGV9/anti-CD123 bispecific antibody or antigen binding fragment thereof any one of embodiments 83 to 91, wherein HC2 and LC2 are humanized.
93. The isolated anti-TRGV9/anti-CD123 bispecific antibody or antigen binding fragment thereof any one of embodiments 83 to 92, wherein the bispecific antibody or antigen binding fragment thereof is an IgG1, an IgG2, an IgG3, or an IgG4 isotype.
94. The isolated anti-TRGV9/anti-CD123 bispecific antibody or antigen binding fragment thereof any one of embodiments 83 to 93, wherein the bispecific antibody or antigen binding fragment thereof is an IgG4 isotype.
95. The isolated anti-TRGV9/anti-CD123 bispecific antibody or antigen binding fragment thereof any one of embodiments 83 to 94, wherein the bispecific antibody or antigen binding fragment thereof induces γδ T cell dependent cytotoxicity of a cancer cell in vitro with an EC₅₀ of less than about 500 pM.
96. The isolated anti-TRGV9/anti-CD123 bispecific antibody or antigen binding fragment thereof embodiment 95, wherein the bispecific antibody or antigen binding fragment thereof induces γδ T cell dependent cytotoxicity of a cancer cell in vitro with an EC₅₀ of less than about 300 pM.
97. The isolated anti-TRGV9/anti-CD123 bispecific antibody or antigen binding fragment thereof embodiment 95, wherein the bispecific antibody or antigen binding fragment thereof induces γδ T cell dependent cytotoxicity of a cancer cell in vitro with an EC₅₀ of less than about 160 pM.
98. The isolated anti-TRGV9/anti-CD123 bispecific antibody or antigen binding fragment thereof any one of embodiments 95 to 97, wherein the EC₅₀ is assessed with a mixture of γδ T effector cells and Kasumi3 AML target cells.
99. The isolated anti-TRGV9/anti-CD123 bispecific antibody or antigen binding fragment thereof embodiment 98, wherein the effector cell to target cell ratio is about 0.01 to 1 to about 5 to 1.
100. The isolated anti-TRGV9/anti-CD123 bispecific antibody or antigen binding fragment thereof embodiment 99, wherein the effector cell to target cell ratio is about 0.1 to 1 to about 2 to 1.
101. The isolated anti-TRGV9/anti-CD123 bispecific antibody or antigen binding fragment thereof embodiment 100, wherein the effector cell to target cell ratio is about 1:1.
102. A method of making the isolated anti-TRGV9/anti-CD123 bispecific antibody or antigen binding fragment thereof any one of embodiments 83 to 101, the method comprising culturing a cell comprising a nucleic acid encoding the anti-TRGV9/anti-CD123 bispecific antibody or antigen binding fragment thereof under conditions to produce the bispecific antibody or antigen binding fragment thereof and recovering the bispecific antibody or antigen binding fragment thereof.
103. An isolated TRGV9 bispecific antibody or antigen epitope binding fragment thereof, wherein the isolated TRGV9 bispecific antibody or antigen epitope binding fragment thereof comprises a binding site for a first antigen and a binding site for a second antigen, wherein the binding site for the first antigen binds a TRGV9 epitope on a γδ T cell and the binding site for the second antigen binds an epitope of the second antigen on a surface of a target cell, and the binding of the TRGV9 epitope on the γδ T cell and the binding of the second antigen epitope on the target cell results in the killing of the target cell.
104. The isolated TRGV9 bispecific antibody or antigen binding fragment thereof, wherein the isolated TRGV9 bispecific antibody or antigen binding fragment thereof comprises:
   a. a HC1;
   b. a HC2;
   c. a LC1; and
   d. a LC2,
   wherein HC1 is associated with LC1 and HC2 is associated with LC2, and
   wherein HC1 comprises a HCDR1, HCDR2, and HCDR3 comprising the amino acid sequences of:
   i. SEQ ID NO:1, SEQ ID NO:2, and SEQ ID NO:3, respectively,
   ii. SEQ ID NO:1, SEQ ID NO:2, and SEQ ID NO:31, respectively,
   iii. SEQ ID NO:1, SEQ ID NO:2, and SEQ ID NO:32, respectively, or
   iv. SEQ ID NO:1, SEQ ID NO:2, and SEQ ID NO:33, respectively,
   and LC1 comprises a LCDR1, LCDR2, and LCDR3 comprising the amino acid sequences of SEQ ID NO:4, SEQ ID NO:5, and SEQ ID NO:6, respectively, to form the binding site for the first antigen, and wherein HC2 and LC2 form the binding site for the second antigen epitope.
105. The isolated TRGV9 bispecific antibody or antigen binding fragment thereof embodiment 104, wherein HC1 comprises an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8.
106. The isolated TRGV9 bispecific antibody or antigen binding fragment thereof embodiment 105, wherein HC1 comprises the amino acid sequence selected from SEQ ID NO:7, SEQ ID NO:34, SEQ ID NO:35, or SEQ ID NO:36, and LC1 comprises the amino acid sequence of SEQ ID NO:8.
107. The isolated TRGV9 bispecific antibody or antigen binding fragment of any one of embodiments 104 to 106, wherein HC1 and LC1 are humanized.
108. The isolated TRGV9 bispecific antibody or antigen binding fragment thereof any one of embodiments 104 to 107, wherein HC2 and LC2 bind to a CD123 epitope.
109. The isolated TRGV9 bispecific antibody or antigen binding fragment thereof embodiment 108, wherein HC2 comprises an amino acid sequence having at least 95% identity to the amino acid sequence of SEQ ID NO:15 and LC2 comprises an amino acid sequence having at least 95% identity to the amino acid sequence of SEQ ID NO:16.
110. The isolated TRGV9 bispecific antibody or antigen binding fragment thereof embodiment 109, wherein HC2 comprises the amino acid sequence of SEQ ID NO:15 and LC2 comprises the amino acid sequence of SEQ ID NO:16.
111. The isolated TRGV9 bispecific antibody or antigen binding fragment thereof any one of embodiments 103 to 110, wherein the bispecific antibody or antigen binding fragment thereof is an IgG1, an IgG2, an IgG3, or an IgG4 isotype.
112. The isolated TRGV9 bispecific antibody or antigen binding fragment thereof any one of embodiments 103 to 111, wherein the bispecific antibody or antigen binding fragment thereof is an IgG4 isotype.
113. The isolated TRGV9 bispecific antibody or antigen binding fragment thereof any one of embodiments 103 to 112, wherein the bispecific antibody or antigen binding fragment thereof induces γδ T cell dependent cytotoxicity of a cancer cell in vitro with an EC₅₀ of less than about 500 pM.
114. The isolated TRGV9 bispecific antibody or antigen binding fragment thereof embodiment 113, wherein the bispecific antibody or antigen binding fragment thereof induces γδ T cell dependent cytotoxicity of a cancer cell in vitro with an EC₅₀ of less than about 300 pM.
115. The isolated TRGV9 bispecific antibody or antigen binding fragment thereof embodiment 114, wherein the bispecific antibody or antigen binding fragment thereof induces γδ T cell dependent cytotoxicity of a cancer cell in vitro with an EC₅₀ of less than about 160 pM.
116. The isolated TRGV9 bispecific antibody or antigen binding fragment thereof any one of embodiments 113 to 115, wherein the EC₅₀ is assessed with a mixture of γδ T effector cells and Kasumi3 AML, target cells.
117. The isolated TRGV9 bispecific antibody or antigen binding fragment thereof embodiment 116 wherein the effector cell to target cell ratio is about 0.01 to 1 to about 5 to 1.
118. The isolated TRGV9 bispecific antibody or antigen binding fragment thereof embodiment 117, wherein the effector cell to target cell ratio is about 0.1 to 1 to about 2 to 1.
119. The isolated TRGV9 bispecific antibody or antigen binding fragment thereof embodiment 118, wherein the effector cell to target cell ratio is about 1:1.
120. An isolated γδ T cell bispecific antibody or antigen binding fragment thereof, wherein the isolated γδ T cell bispecific antibody or antigen binding fragment thereof comprises a binding site for a first antigen epitope and a binding site for a second antigen epitope, wherein the binding site for the first antigen epitope binds a first antigen on a γδ T cell and the binding site for the second antigen epitope binds the second antigen epitope on a surface of a target cell, and the binding of the first antigen epitope on the γδ T cell and the binding of the second antigen epitope on the target cell results in the killing of the target cell.
121. An isolated nucleic acid encoding a TRGV9 bispecific antibody or antigen binding fragment thereof, the isolated TRGV9 bispecific antibody or antigen binding fragment thereof comprising:
   a. a HC1;
   b. a HC2;
   c. a LC1; and
   d. a LC2,
   wherein HC1 is associated with LC1 and HC2 is associated with LC2, and
   wherein HC1 comprises a HCDR1, HCDR2, and HCDR3 comprising the amino acid sequences of:
   i. SEQ ID NO:1, SEQ ID NO:2, and SEQ ID NO:3, respectively,
   ii. SEQ ID NO:1, SEQ ID NO:2, and SEQ ID NO:31, respectively,
   iii. SEQ ID NO:1, SEQ ID NO:2, and SEQ ID NO:32, respectively, or
   iv. SEQ ID NO:1, SEQ ID NO:2, and SEQ ID NO:33, respectively,
   and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen.
122. The isolated nucleic acid of embodiment 121, wherein HC1 comprises an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8.
123. The isolated nucleic acid of embodiment 122, wherein HC1 comprises the amino acid sequence selected from SEQ ID NO:7, SEQ ID NO:34, SEQ ID NO:35, or SEQ ID NO:36, and LC1 comprises the amino acid sequence of SEQ ID NO:8.
124. The isolated nucleic acid of any one of embodiments 121 to 123, wherein the binding site for a first antigen binds to TRGV9 on a γδ T cell.
125. The isolated nucleic acid of any one of embodiments 121 to 124, wherein the binding site for a second antigen binds to a cancer antigen present on the surface of a cancer cell.
126. The isolated nucleic acid of embodiment 125, wherein the binding of the bispecific antibody to TRGV9 present on the surface of the γδ T cell and the binding of the cancer antigen present on the surface of the cancer cell results in the killing of the cancer cell.
127. The isolated nucleic acid of any one of embodiments 121 to 126, wherein HC1 and LC1 are humanized.
128. The isolated nucleic acid of any one of embodiments 121 to 127, wherein HC2 and LC2 bind to CD123.
129. The isolated nucleic acid of any one of embodiments 121 to 128, wherein the bispecific antibody or antigen binding fragment thereof is an IgG1, an IgG2, an IgG3, or an IgG4 isotype.
130. The isolated nucleic acid of any one of embodiments 121 to 129, wherein the bispecific antibody or antigen binding fragment thereof is an IgG4 isotype.
131. The isolated nucleic acid of any one of embodiments 121 to 130, wherein the bispecific antibody or antigen binding fragment thereof induces γδ T cell dependent cytotoxicity of a cancer cell in vitro with an EC₅₀ of less than about 500 pM.
132. The isolated nucleic acid of embodiment 131, wherein the bispecific antibody or antigen binding fragment thereof induces γδ T cell dependent cytotoxicity of a cancer cell in vitro with an EC₅₀ of less than about 300 pM.
133. The isolated nucleic acid of embodiment 131, wherein the bispecific antibody or antigen binding fragment thereof induces γδ T cell dependent cytotoxicity of a cancer cell in vitro with an EC₅₀ of less than about 160 pM.
134. The isolated nucleic acid of any one of embodiments 131 to 133, wherein the EC₅₀ is assessed with a mixture of γδ T effector cells and Kasumi3 AML target cells.
135. The isolated nucleic acid of embodiment 134, wherein the effector cell to target cell ratio is about 0.01 to 1 to about 5 to 1.
136. The isolated nucleic acid of embodiment 135 wherein the effector cell to target cell ratio is about 0.1 to 1 to about 2 to 1.
137. The isolated nucleic acid of embodiment 136, wherein the effector cell to target cell ratio is about 1:1.
138. The isolated nucleic acid of any one of embodiments 121 to 137, wherein the bispecific antibody or antigen binding fragment thereof is multivalent.
139. The isolated nucleic acid of embodiment 138, wherein the bispecific antibody or antigen binding fragment thereof is capable of binding at least three antigens.
140. The isolated nucleic acid of embodiment 138, wherein the bispecific antibody or antigen binding fragment thereof is capable of binding at least five antigens.
141. A vector comprising the isolated nucleic acid of any one of embodiments 121 to 140.
142. A host cell comprising the vector of embodiment 141.
143. A kit comprising the vector of embodiment 141 and packaging for the same.
144. A pharmaceutical composition comprising an isolated TRGV9 bispecific antibody or antigen binding fragment thereof, the isolated TRGV9 bispecific antibody or antigen binding fragment thereof comprising:
   a. a HC1;
   b. a HC2;
   c. a LC1; and
   d. a LC2,
   wherein HC1 is associated with LC1 and HC2 is associated with LC2, and
   wherein HC1 comprises a HCDR1, HCDR2, and HCDR3 comprising the amino acid sequences of:
   i. SEQ ID NO:1, SEQ ID NO:2, and SEQ ID NO:3, respectively,
   ii. SEQ ID NO:1, SEQ ID NO:2, and SEQ ID NO:31, respectively,
   iii. SEQ ID NO:1, SEQ ID NO:2, and SEQ ID NO:32, respectively, or
   iv. SEQ ID NO:1, SEQ ID NO:2, and SEQ ID NO:33, respectively,
   and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen,
   and a pharmaceutically acceptable carrier.
145. The pharmaceutical composition of embodiment 144, wherein HC1 comprises an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8.
146. The pharmaceutical composition of embodiment 145, wherein HC1 comprises the amino acid sequence selected from SEQ ID NO:7, SEQ ID NO:34, SEQ ID NO:35, or SEQ ID NO:36, and LC1 comprises the amino acid sequence of SEQ ID NO:8.
147. The pharmaceutical composition of any one of embodiments 144 to 146, wherein the binding site for a first antigen binds to TRGV9 on a γδ T cell.
148. The pharmaceutical composition of any one of embodiments 144 to 147, wherein the binding site for a second antigen binds to a cancer antigen present on the surface of a cancer cell.
149. The pharmaceutical composition of embodiment 148, wherein the binding of the bispecific antibody to TRGV9 present on the surface of the γδ T cell and the binding of the cancer antigen present on the surface of the cancer cell results in the killing of the cancer cell.
150. The pharmaceutical composition of any one of embodiments 144 to 149, wherein HC1 and LC1 are humanized.
151. The pharmaceutical composition of any one of embodiments 144 to 150, wherein HC2 and LC2 bind to CD123.
152. The pharmaceutical composition of any one of embodiments 144 to 151, wherein the bispecific antibody or antigen binding fragment thereof is an IgG1, an IgG2, an IgG3, or an IgG4 isotype.
153. A method of directing a Vγ9-expressing γδ T cell to a cancer cell, the method comprising contacting a Vγ9-expressing γδ T cell with the pharmaceutical composition of any one of embodiments 144 to 152, wherein contacting the Vγ9-expressing γδ T cell with the pharmaceutical composition directs the Vγ9-expressing γδ T cell to a cancer cell.
154. A method of inhibiting growth or proliferation of cancer cells expressing a cancer antigen on the cell surface, the method comprising contacting the cancer cells with the pharmaceutical composition of any one of embodiments 144 to 152, wherein contacting the cancer cells with the pharmaceutical composition inhibits growth or proliferation of the cancer cells.
155. The method of embodiment 154, wherein the cancer cell is in the presence of a Vγ9-expressing γδ T cell while in contact with anti-TRGV9 bispecific antibody or antigen binding fragment thereof.
156. A method for treating a cancer in a subject in need thereof, the method comprising:
   a. identifying a subject in need of cancer treatment; and
   b. administering to the subject in need thereof the pharmaceutical composition of any one of embodiments 144 to 152,
   wherein administering the pharmaceutical composition to the subject in need thereof treats the cancer in the subject.
157. A method of activating a Vγ9-expressing γδ T cell, the method comprising contacting the Vγ9-expressing γδ T cell with the pharmaceutical composition of any one of embodiments 144 to 152, wherein contacting the Vγ9-expressing γδ T cell with the pharmaceutical composition results in an increase in CD69, CD25, and/or Granzyme B expression as compared to a control Vγ9-expressing γδ T cell.
158. A method of producing the pharmaceutical composition of any one of embodiments 144 to 152, the method comprising combining the bispecific antibody or antigen binding fragment thereof with a pharmaceutically acceptable carrier to obtain the pharmaceutical composition.
159. An antibody that binds to TRGV9, wherein the antibody comprises:
   i. a VH comprising a VH CDR1 having an amino acid sequence of SEQ ID NO:1, a VH CDR2 having an amino acid sequence of SEQ ID NO:76, and a VH CDR3 having an amino acid sequence of SEQ ID NO:3; and
   ii. a VL comprising a VL CDR1 having an amino acid sequence of SEQ ID NO:77, a VL CDR2 having an amino acid sequence of SEQ ID NO:5, and a VL CDR3 having an amino acid sequence of SEQ ID NO:6.
160. The antibody of embodiment 159, wherein the antibody comprises a VH having an amino acid sequence of SEQ ID NO:65.
161. The antibody of embodiment 159, wherein the antibody comprises a VL having an amino acid sequence of SEQ ID NO:66.
162. The antibody of embodiment 159, wherein the antibody comprises a VH
having an amino acid sequence of SEQ ID NO:65, and a VL having an amino acid sequence of SEQ ID NO:66.
163. The antibody of embodiment 159, wherein the antibody comprises a VH having an amino acid sequence of SEQ ID NO:67.
164. The antibody of embodiment 159, wherein the antibody comprises a VL having an amino acid sequence of SEQ ID NO:68.
165. The antibody of embodiment 159, wherein the antibody comprises a VH having an amino acid sequence of SEQ ID NO:67, and a VL having an amino acid sequence of SEQ ID NO:68.
166. An antibody that binds to TRGV9, wherein the antibody comprises:
   i. a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and
   ii. a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6.
167. The antibody of embodiment 166, wherein the antibody comprises a VH having an amino acid sequence of SEQ ID NO:65.
168. The antibody of embodiment 166, wherein the antibody comprises a VL having an amino acid sequence of SEQ ID NO:66.
169. The antibody of embodiment 166, wherein the antibody comprises a VH having an amino acid sequence of SEQ ID NO:65, and a VL having an amino acid sequence of SEQ ID NO:66.
170. The antibody of embodiment 166, wherein the antibody comprises a VH having an amino acid sequence of SEQ ID NO:67.
171. The antibody of embodiment 166, wherein the antibody comprises a VL having an amino acid sequence of SEQ ID NO:68.
172. The antibody of embodiment 166, wherein the antibody comprises a VH having an amino acid sequence of SEQ ID NO:67, and a VL having an amino acid sequence of SEQ ID NO:68.
173. An antibody that binds to TRGV9, wherein the antibody comprises:
   i. a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100; and
   ii. a VL comprising a VL CDR1 having an amino acid sequence of SEQ ID NO:101, a VL CDR2 having an amino acid sequence of SEQ ID NO:102, and a VL CDR3 having an amino acid sequence of SEQ ID NO:103.
174. The antibody of embodiment 173, wherein the antibody comprises a VH having an amino acid sequence of SEQ ID NO:104.
175. The antibody of embodiment 173, wherein the antibody comprises a VL having an amino acid sequence of SEQ ID NO:105.
176. The antibody of embodiment 173, wherein the antibody comprises a VH having an amino acid sequence of SEQ ID NO:104, and a VL having an amino acid sequence of SEQ ID NO:105.
177. An antibody that binds to TRGV9, wherein the antibody comprises:
   i. a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109; and
   ii. a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO:112.
178. The antibody of embodiment 177, wherein the antibody comprises a VH having an amino acid sequence of SEQ ID NO:113.
179. The antibody of embodiment 177, wherein the antibody comprises a VL having an amino acid sequence of SEQ ID NO:114.
180. The antibody of embodiment 177, wherein the antibody comprises a VH having an amino acid sequence of SEQ ID NO:113, and a VL having an amino acid sequence of SEQ ID NO:114.
181. An antibody that binds to TRGV9, wherein the antibody comprises:
   i. a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119; and
   ii. a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO:122.
182. The antibody of embodiment 181, wherein the antibody comprises a VH having an amino acid sequence of SEQ ID NO:123.
183. The antibody of embodiment 181, wherein the antibody comprises a VL having an amino acid sequence of SEQ ID NO:124.
184. The antibody of embodiment 181, wherein the antibody comprises a VH having an amino acid sequence of SEQ ID NO:123, and a VL having an amino acid sequence of SEQ ID NO:124.
185. An antibody that binds to TRGV9, wherein the antibody comprises:
   i. a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO:129; and
   ii. a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO:132.
186. The antibody of embodiment 185, wherein the antibody comprises a VH having an amino acid sequence of SEQ ID NO:133.
187. The antibody of embodiment 185, wherein the antibody comprises a VL having an amino acid sequence of SEQ ID NO:134.
188. The antibody of embodiment 185, wherein the antibody comprises a VH having an amino acid sequence of SEQ ID NO:133, and a VL having an amino acid sequence of SEQ ID NO:134.
189. The antibody of any one of embodiments 159 to 188, wherein the TRGV9 is present on the surface of a γδ T cell.
190. The antibody of any one of embodiments 159 to 188, wherein the antibody is a humanized antibody.
191. The antibody of any one of embodiments 159 to 188, wherein the antibody is an IgG antibody.
192. The antibody of embodiment 191, wherein the IgG antibody is an IgG1, IgG2, IgG3, IgG4 antibody.
193. The antibody of any one of embodiments 159 to 192, wherein the antibody is a bispecific antibody.
194. The antibody of any one of embodiments 159 to 192, wherein the antibody is multivalent.
195. The antibody of embodiment 194, wherein the antibody is capable of binding at least three antigens.
196. The antibody of embodiment 194, wherein the antibody is capable of binding at least five antigens.
197. A nucleic acid encoding the antibody of any one of embodiments 159 to 196.
198. A vector comprising the nucleic acid of embodiment 197.
199. A host cell comprising the vector of embodiment 197.
200. A kit comprising the vector of embodiment 197 and packaging for the same.
201. A pharmaceutical composition comprising the bispecific antibody of any one of embodiments 159 to 196, and a pharmaceutically acceptable carrier.
202. A method of producing the pharmaceutical composition of embodiment 201, comprising combining the antibody with a pharmaceutically acceptable carrier to obtain the pharmaceutical composition.
203. A method of activating a γδ T cell expressing TRGV9, comprising contacting the γδ T cell with the antibody of any one of embodiments 159 to 196.
204. The method of embodiment 203, wherein the contacting results in an increase in CD69, CD25, and/or Granzyme B expression, as compared to a control γδ T cell expressing TRGV9.
205. A bispecific antibody comprising a first binding domain that binds to a TRGV9 antigen, and a second binding domain that binds to an antigen on the surface of a cancer cell, wherein the first binding domain comprises:
   i. a VH comprising a VH CDR1 having an amino acid sequence of SEQ ID NO:1, a VH CDR2 having an amino acid sequence of SEQ ID NO:76, and a VH CDR3 having an amino acid sequence of SEQ ID NO:3; and
   ii. a VL comprising a VL CDR1 having an amino acid sequence of SEQ ID NO:77, a VL CDR2 having an amino acid sequence of SEQ ID NO:5, and a VL CDR3 having an amino acid sequence of SEQ ID NO:6.
206. The bispecific antibody of embodiment 205, wherein the antibody comprises a VH having an amino acid sequence of SEQ ID NO:65.
207. The bispecific antibody of embodiment 205, wherein the antibody comprises a VL having an amino acid sequence of SEQ ID NO:66.
208. The bispecific antibody of embodiment 205, wherein the antibody comprises a VH having an amino acid sequence of SEQ ID NO:65, and a VL having an amino acid sequence of SEQ ID NO:66.
209. The bispecific antibody of embodiment 205, wherein the antibody comprises a VH having an amino acid sequence of SEQ ID NO:67.
210. The bispecific antibody of embodiment 205, wherein the antibody comprises a VL having an amino acid sequence of SEQ ID NO:68.
211. The bispecific antibody of embodiment 205, wherein the antibody comprises a VH having an amino acid sequence of SEQ ID NO:67, and a VL having an amino acid sequence of SEQ ID NO:68.
212. A bispecific antibody comprising a first binding domain that binds to a TRGV9 antigen, and a second binding domain that binds to an antigen on the surface of a cancer cell, wherein the first binding domain comprises:
   i. a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and
   ii. a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6.
213. The bispecific antibody of embodiment 212, wherein the antibody comprises a VH having an amino acid sequence of SEQ ID NO:65.
214. The bispecific antibody of embodiment 212, wherein the antibody comprises a VL having an amino acid sequence of SEQ ID NO:66.
215. The bispecific antibody of embodiment 212, wherein the antibody comprises a VH having an amino acid sequence of SEQ ID NO:65, and a VL having an amino acid sequence of SEQ ID NO:66.
216. The bispecific antibody of embodiment 212, wherein the antibody comprises a VH having an amino acid sequence of SEQ ID NO:67.
217. The bispecific antibody of embodiment 212, wherein the antibody comprises a VL having an amino acid sequence of SEQ ID NO:68.
218. The bispecific antibody of embodiment 212, wherein the antibody comprises a VH having an amino acid sequence of SEQ ID NO:67, and a VL having an amino acid sequence of SEQ ID NO:68.
219. The bispecific antibody of any one of embodiments 205 to 218, wherein the antigen on the surface of the cancer cell is a tumor-specific antigen, a tumor associated antigen, or a neoantigen.
220. The bispecific antibody of any one of embodiments 205 to 218, wherein the TRGV9 is present on the surface of a γδ T cell.
221. The bispecific antibody of any one of embodiments 205 to 218, wherein the TRGV9 is present on the surface of a γδ T cell, and the antigen expressed on the surface of the cancer cell is a cancer antigen.
222. The bispecific antibody of embodiment 221, wherein the target cell is killed when the bispecific antibody binds to the TRGV9 on the surface of the γδ T cell and the antigen on the surface of the target cell.
223. The bispecific antibody of any one of embodiments 205 to 222, wherein the first binding domain is humanized, the second binding domain is humanized, or both the first binding domain and the second binding domain are humanized.
224. The bispecific antibody of any one of embodiments 205 to 223, wherein the bispecific antibody is an IgG antibody.
225. The bispecific antibody of embodiment 224, wherein the IgG antibody is an IgG1, IgG2, IgG3, IgG4 antibody.
226. A pharmaceutical composition comprising the bispecific antibody of any one of embodiments 205 to 225, and a pharmaceutically acceptable carrier.
227. A method of directing a γδ T cell expressing TRGV9 to a cancer cell, the method comprising contacting the γδ T cell with the bispecific antibody of any one of embodiments 205 to 225, wherein the contacting directs the γδ T cell to the cancer cell.
228. A method of inhibiting growth or proliferation of cancer cells expressing a cancer antigen on the cell surface, the method comprising contacting the cancer cells with the bispecific antibody of any one of embodiments 205 to 225, wherein contacting the cancer cells with the pharmaceutical composition inhibits growth or proliferation of the cancer cells.
229. The method of embodiment 228, wherein the cancer cells are in the presence of a γδ T cell expressing TRGV9 while in contact with the bispecific antibody.
230. A method for eliminating cancer cells in a subject, comprising administering an effective amount of the bispecific antibody of any one of embodiments 205 to 225 to the subject.
231. The method of embodiment 214, wherein the subject is a subject in need thereof.
232. The method of embodiments 214 or 215, wherein the subject is a human.
233. A method of activating a γδ T cell expressing TRGV9, comprising contacting the γδ T cell with the bispecific antibody of any one of embodiments 205 to 225.
234. A bispecific antibody comprising:
   a. a first binding domain that binds to a TRGV9 antigen, and
   b. a second binding domain that binds to an antigen that is not TRGV9.
235. The bispecific antibody of embodiment 234, wherein the first binding domain comprises
   (A) a HC1 comprising a HCDR1, HCDR2, and HCDR3 comprising the amino acid sequences of:
      i. SEQ ID NO:1, SEQ ID NO:2, and SEQ ID NO:3, respectively,
      ii. SEQ ID NO:1, SEQ ID NO:2, and SEQ ID NO:31, respectively,
      iii. SEQ ID NO:1, SEQ ID NO:2, and SEQ ID NO:32, respectively, or
      iv. SEQ ID NO:1, SEQ ID NO:2, and SEQ ID NO:33, respectively,
      and a LC1 comprising a LCDR1, LCDR2, and LCDR3 comprising the amino acid sequences of SEQ ID NO:4, SEQ ID NO:5, and SEQ ID NO:6, respectively, to form a binding site for the TRGV9 antigen;
   (B) a HC1 comprising a HCDR1, HCDR2, and HCDR3 comprising the amino acid sequences of SEQ ID NO:1, SEQ ID NO:76, and SEQ ID NO:3, respectively,
      and a LC1 comprising a LCDR1, LCDR2, and LCDR3 comprising the amino acid sequences of SEQ ID NO:77, SEQ ID NO:5, and SEQ ID NO:6, respectively, to form a binding site for the TRGV9 antigen;
   (C) a HC1 comprising a HCDR1, HCDR2, and HCDR3 comprising the amino acid sequences of SEQ ID NO:60, SEQ ID NO:61, and SEQ ID NO:62, respectively,
      and a LC1 comprising a LCDR1, LCDR2, and LCDR3 comprising the amino acid sequences of SEQ ID NO:63, SEQ ID NO:64, and SEQ ID NO:6, respectively, to form a binding site for the TRGV9 antigen;
   (D) a HC1 comprising a HCDR1, HCDR2, and HCDR3 comprising the amino acid sequences of SEQ ID NO:89, SEQ ID NO:90, and SEQ ID NO:91, respectively,
      and a LC1 comprising a LCDR1, LCDR2, and LCDR3 comprising the amino acid sequences of SEQ ID NO:92, SEQ ID NO:93, and SEQ ID NO:94, respectively, to form a binding site for the TRGV9 antigen;
   (E) a HC1 comprising a HCDR1, HCDR2, and HCDR3 comprising the amino acid sequences of SEQ ID NO:98, SEQ ID NO:99, and SEQ ID NO:100, respectively,
      and a LC1 comprising a LCDR1, LCDR2, and LCDR3 comprising the amino acid sequences of SEQ ID NO:101, SEQ ID NO:102, and SEQ ID NO:103, respectively, to form a binding site for the TRGV9 antigen;
   (F) a HC1 comprising a HCDR1, HCDR2, and HCDR3 comprising the amino acid sequences of SEQ ID NO:107, SEQ ID NO:108, and SEQ ID NO:109, respectively,
      and a LC1 comprising a LCDR1, LCDR2, and LCDR3 comprising the amino acid sequences of SEQ ID NO:110, SEQ ID NO:111, and SEQ ID NO:112, respectively, to form a binding site for the TRGV9 antigen;
   (G) a HC1 comprising a HCDR1, HCDR2, and HCDR3 comprising the amino acid sequences of SEQ ID NO:117, SEQ ID NO:118, and SEQ ID NO:119, respectively,
      and a LC1 comprising a LCDR1, LCDR2, and LCDR3 comprising the amino acid sequences of SEQ ID NO:120, SEQ ID NO:121, and SEQ ID NO:122, respectively, to form a binding site for the TRGV9 antigen; or
   (H) a HC1 comprising a HCDR1, HCDR2, and HCDR3 comprising the amino acid sequences of SEQ ID NO:127, SEQ ID NO:128, and SEQ ID NO:129, respectively,
      and a LC1 comprising a LCDR1, LCDR2, and LCDR3 comprising the amino acid sequences of SEQ ID NO:130, SEQ ID NO:131, and SEQ ID NO:132, respectively, to form a binding site for the TRGV9 antigen.
236. The bispecific antibody of embodiment 234 or 235, wherein the first binding domain has a heavy chain having an amino acid sequence of SEQ ID NO:69.
237. The bispecific antibody of embodiment 234 or 235, wherein the first binding domain has a light chain having an amino acid sequence of SEQ ID NO:24.
238. The bispecific antibody of embodiment 234 or 235, wherein the first binding domain has a heavy chain having an amino acid sequence of SEQ ID NO:69, and a light chain having an amino acid sequence of SEQ ID NO:24.
239. The bispecific antibody of embodiment 234 or 235, wherein the first binding domain has a heavy chain having an amino acid sequence of SEQ ID NO:71.
240. The bispecific antibody of embodiment 234 or 235, wherein the first binding domain has a light chain having an amino acid sequence of SEQ ID NO:72.
241. The bispecific antibody of embodiment 234 or 235, wherein the first binding domain has a heavy chain having an amino acid sequence of SEQ ID NO:71, and a light chain having an amino acid sequence of SEQ ID NO:72.
242. The bispecific antibody of embodiment 234 or 235, wherein the first binding domain has a heavy chain having an amino acid sequence of SEQ ID NO:74.
243. The bispecific antibody of embodiment 234 or 235, wherein the first binding domain has a light chain having an amino acid sequence of SEQ ID NO:75.
244. The bispecific antibody of embodiment 234 or 235, wherein the first binding domain has a heavy chain having an amino acid sequence of SEQ ID NO:74, and a light chain having an amino acid sequence of SEQ ID NO:75.
245. The bispecific antibody of embodiment 234 or 235, wherein the first binding domain has a heavy chain having an amino acid sequence of SEQ ID NO:115.
246. The bispecific antibody of embodiment 234 or 235, wherein the first binding domain has a light chain having an amino acid sequence of SEQ ID NO:116.
247. The bispecific antibody of embodiment 234 or 235, wherein the first binding domain has a heavy chain having an amino acid sequence of SEQ ID NO:115, and a light chain having an amino acid sequence of SEQ ID NO:116.
248. The bispecific antibody of embodiment 234 or 235, wherein the first binding domain has a heavy chain having an amino acid sequence of SEQ ID NO:125.
249. The bispecific antibody of embodiment 234 or 235, wherein the first binding domain has a light chain having an amino acid sequence of SEQ ID NO:126.
250. The bispecific antibody of embodiment 234 or 235, wherein the first binding domain has a heavy chain having an amino acid sequence of SEQ ID NO:125, and a light chain having an amino acid sequence of SEQ ID NO:126.
251. The bispecific antibody of embodiment 234 or 235, wherein the first binding domain has a heavy chain having an amino acid sequence of SEQ ID NO:135.
252. The bispecific antibody of embodiment 234 or 235, wherein the first binding domain has a light chain having an amino acid sequence of SEQ ID NO:136.
253. The bispecific antibody of embodiment 234 or 235, wherein the first binding domain has a heavy chain having an amino acid sequence of SEQ ID NO:135, and a light chain having an amino acid sequence of SEQ ID NO:136.
254. The bispecific antibody of any one of embodiments 234 to 253, wherein the second binding domain binds to CD33.
255. The bispecific antibody of embodiment 254, wherein the second binding domain comprises a HC1 comprising a HCDR1, HCDR2, and HCDR3 comprising the amino acid sequences of SEQ ID NO:37, SEQ ID NO:38, and SEQ ID NO:39, respectively, and LC1 comprises a LCDR1, LCDR2, and LCDR3 comprising the amino acid sequences of SEQ ID NO:40, SEQ ID NO:41, and SEQ ID NO:42, respectively, to form a binding site for the CD33 antigen.
256. The bispecific antibody of embodiment 254, wherein the second binding domain comprises a VH having an amino acid sequence of SEQ ID NO:43.
257. The bispecific antibody of embodiment 254, wherein the second binding domain comprises a VL having an amino acid sequence of SEQ ID NO:44.
258. The bispecific antibody of embodiment 254, wherein the second binding domain comprises a VH having an amino acid sequence of SEQ ID NO:43, and a VL having an amino acid sequence of SEQ ID NO:44.
259. The bispecific antibody of embodiment 254, wherein the second binding domain has a heavy chain having an amino acid sequence of SEQ ID NO:47.
260. The bispecific antibody of embodiment 254, wherein the second binding domain has light chain having an amino acid sequence of SEQ ID NO:48.
261. The bispecific antibody of embodiment 254, wherein the second binding domain has a heavy chain having an amino acid sequence of SEQ ID NO:47, and light chain having an amino acid sequence of SEQ ID NO:48.
262. The bispecific antibody of embodiment 254, wherein the second binding domain has an amino acid sequence of SEQ ID NO:45.
263. The bispecific antibody of any one of embodiments 234 to 253, wherein the second binding domain binds to TRBC1.
264. The bispecific antibody of embodiment 263, wherein the second binding domain comprises a HC1 comprising a HCDR1, HCDR2, and HCDR3 comprising the amino acid sequences of SEQ ID NO:49, SEQ ID NO:50, and SEQ ID NO:51, respectively, and LC1 comprises a LCDR1, LCDR2, and LCDR3 comprising the amino acid sequences of SEQ ID NO:52, SEQ ID NO:53, and SEQ ID NO:54, respectively, to form a binding site for the TRBC1 antigen.
265. The bispecific antibody of embodiment 263, wherein the second binding domain comprises a VH having an amino acid sequence of SEQ ID NO:55.
266. The bispecific antibody of embodiment 263, wherein the second binding domain comprises a VL having an amino acid sequence of SEQ ID NO:56.
267. The bispecific antibody of embodiment 263, wherein the second binding domain comprises a VH having an amino acid sequence of SEQ ID NO:55, and a VL having an amino acid sequence of SEQ ID NO:56.
268. The bispecific antibody of embodiment 263, wherein the second binding domain has a heavy chain having an amino acid sequence of SEQ ID NO:58.
269. The bispecific antibody of embodiment 263, wherein the second binding domain has light chain having an amino acid sequence of SEQ ID NO:59.
270. The bispecific antibody of embodiment 263, wherein the second binding domain has a heavy chain having an amino acid sequence of SEQ ID NO:58, and light chain having an amino acid sequence of SEQ ID NO:59.
271. The bispecific antibody of embodiment 263, wherein the second binding domain has an amino acid sequence of SEQ ID NO:57.
272. The bispecific antibody of any one of embodiments 234 to 253, wherein the second binding domain binds to BCMA.
273. The bispecific antibody of embodiment 272, wherein the second binding domain comprises a HC1 comprising a HCDR1, HCDR2, and HCDR3 comprising the amino acid sequences of SEQ ID NO:37, SEQ ID NO:38, and SEQ ID NO:39, respectively, and LC1 comprises a LCDR1, LCDR2, and LCDR3 comprising the amino acid sequences of SEQ ID NO:40, SEQ ID NO:41, and SEQ ID NO:42, respectively, to form a binding site for the BCMA antigen.
274. The bispecific antibody of embodiment 272, wherein the second binding domain comprises a VH having an amino acid sequence of SEQ ID NO:143.
275. The bispecific antibody of embodiment 272, wherein the second binding domain comprises a VL having an amino acid sequence of SEQ ID NO:144.
276. The bispecific antibody of embodiment 272, wherein the second binding domain comprises a VH having an amino acid sequence of SEQ ID NO:143, and a VL having an amino acid sequence of SEQ ID NO:144.
277. The bispecific antibody of embodiment 272, wherein the second binding domain has a heavy chain having an amino acid sequence of SEQ ID NO:146.
278. The bispecific antibody of embodiment 272, wherein the second binding domain has light chain having an amino acid sequence of SEQ ID NO:147.
279. The bispecific antibody of embodiment 272, wherein the second binding domain has a heavy chain having an amino acid sequence of SEQ ID NO:146, and light chain having an amino acid sequence of SEQ ID NO:147.
280. The bispecific antibody of embodiment 272, wherein the second binding domain has an amino acid sequence of SEQ ID NO:145.
281. The bispecific antibody of embodiment 234 or 235, wherein the second binding domain has an amino acid sequence of SEQ ID NO:17.
282. The bispecific antibody of embodiment 234 or 235, wherein the second binding domain has an amino acid sequence of SEQ ID NO:70.
283. The bispecific antibody of embodiment 234 or 235, wherein the second binding domain has an amino acid sequence of SEQ ID NO:73.
284. The bispecific antibody of embodiment 281, wherein the first binding domain has an amino acid sequence of SEQ ID NO:17, and the second binding domain has an amino acid sequence of SEQ ID NO:45.
285. The bispecific antibody of embodiment 282, wherein the first binding domain has an amino acid sequence of SEQ ID NO:70, and the second binding domain has an amino acid sequence of SEQ ID NO:45.
286. The bispecific antibody of embodiment 283, wherein the first binding domain has an amino acid sequence of SEQ ID NO:73, and the second binding domain has an amino acid sequence of SEQ ID NO:45.
287. The bispecific antibody of embodiment 281, wherein the first binding domain has an amino acid sequence of SEQ ID NO:17, and the second binding domain has an amino acid sequence of SEQ ID NO:57.
288. The bispecific antibody of embodiment 282, wherein the first binding domain has an amino acid sequence of SEQ ID NO:70, and the second binding domain has an amino acid sequence of SEQ ID NO:57.
289. The bispecific antibody of embodiment 283, wherein the first binding domain has an amino acid sequence of SEQ ID NO:73, and the second binding domain has an amino acid sequence of SEQ ID NO:57.
290. The bispecific antibody of embodiment 281, wherein the first binding domain has an amino acid sequence of SEQ ID NO:17, and the second binding domain has an amino acid sequence of SEQ ID NO:145.
291. The bispecific antibody of embodiment 282, wherein the first binding domain has an amino acid sequence of SEQ ID NO:70, and the second binding domain has an amino acid sequence of SEQ ID NO:145.
292. The bispecific antibody of embodiment 283, wherein the first binding domain has an amino acid sequence of SEQ ID NO:73, and the second binding domain has an amino acid sequence of SEQ ID NO:145.
293. The bispecific antibody of any one of embodiments 254 to 262 or 284 to 286, wherein the bispecific antibody induces γδ T cell dependent cytotoxicity of a cell expressing CD33 in vitro with an EC₅₀ of less than about 160 pM.
294. The bispecific antibody of antibody of any one of embodiments 254 to 262 or 284 to 286, wherein the EC₅₀ is assessed with a mixture of γδ T effector cells and target cells expressing the CD33 antigen.
295. The bispecific antibody of embodiment 294, wherein the effector cell to target cell ratio is about 0.01 to 1 to about 10 to 1.
296. The bispecific antibody of embodiment 294, wherein the effector cell to target cell ratio is about 0.01 to 1 to about 5 to 1.
297. The bispecific antibody of embodiment 294, wherein the effector cell to target cell ratio is about 0.1 to 1 to about 2 to 1.
298. The bispecific antibody of embodiment 294, wherein the effector cell to target cell ratio is about 1:1.
299. The bispecific antibody of any one of embodiments 254 to 262 or 284 to 286, wherein the bispecific antibody is multivalent.
300. The bispecific antibody of any one of embodiments 254 to 262 or 284 to 286, wherein the bispecific antibody is capable of binding at least three antigens.
301. The bispecific antibody of embodiment 300, wherein the bispecific antibody is capable of binding at least five antigens.
302. A bispecific antibody comprising: a first means capable of binding TRGV9 on the surface of the γδ T cell; and a second means capable of binding a CD33 antigen.
303. The bispecific antibody of embodiment 290, wherein the CD33 antigen is on the surface of a cell.
304. A nucleic acid encoding the bispecific antibody of any one of embodiments antibody of any one of embodiments 254 to 262 or 284 to 286.
305. A vector comprising the nucleic acid of embodiment 304.
306. A host cell comprising the vector of embodiment 305.
307. A kit comprising the vector of embodiment 305 and packaging for the same.
308. A pharmaceutical composition comprising the bispecific antibody of any one of embodiments antibody of any one of embodiments 254 to 262 or 284 to 286, and a pharmaceutically acceptable carrier.
309. A method of producing the pharmaceutical composition of embodiment 308, comprising combining the bispecific antibody with a pharmaceutically acceptable carrier to obtain the pharmaceutical composition.
310. A process for making an antibody that binds to more than one target molecule, the process comprising: a step for performing a function of obtaining a binding domain capable of binding to a TRGV9 antigen on a γδ T cell; a step for performing a function of obtaining a binding domain capable of binding to a CD33 antigen on the surface of a second cell; and a step for performing a function of providing an antibody capable of binding to a TRGV9 antigen on a γδ T cell and a CD33 antigen on the surface of a second cell.
311. The process of embodiment 310, wherein the step for performing a function of obtaining a binding domain capable of binding to a CD33 antigen on the surface of a second cell is repeated n times and further comprising n steps for performing a function of providing a binding domain capable of binding to a TRGV9 antigen on a γδ T cell and n number of target molecules, wherein n is at least 2.
312. A method of directing a γδ T cell expressing TRGV9 to a second cell expressing CD33, the method comprising contacting the γδ T cell with the bispecific antibody of any one of embodiments 254 to 262 or 284 to 286, wherein the contacting directs the γδ T cell to the cell expressing CD33.
313. A method of inhibiting growth or proliferation of cells expressing a CD33 antigen on the cell surface, the method comprising contacting the cells expressing CD33 with the bispecific antibody of any one of embodiments 254 to 262 or 284 to 286, wherein contacting the cells expressing CD33 with the pharmaceutical composition inhibits growth or proliferation of the cells expressing the CD33 antigen.
314. The method of embodiment 313, wherein the cells expressing the CD33 antigen are in the presence of a γδ T cell expressing TRGV9 while in contact with the bispecific antibody.
315. A method for eliminating cells expressing CD33, or treating a disorder caused all or in part by cells expressing CD33, in a subject, comprising administering an effective amount of the bispecific antibody of any one of embodiments 254 to 262 or 284 to 286 to the subject.
316. The method of embodiment 315, wherein the disorder is a leukemia.
317. The method of embodiment 315, wherein the disorder is a lymphoma.
318. The method of any one of embodiments 315 to 317, wherein the subject is a subject in need thereof.
319. The method of any one of embodiments 315 to 318, wherein the subject is a human.
320. A method of activating a γδ T cell expressing TRGV9, comprising contacting the γδ T cell with the bispecific antibody of any one of embodiments 254 to 262 or 284 to 286.
321. The method of embodiment 320, wherein the contacting results in an increase in CD69, CD25, and/or Granzyme B expression, as compared to a control γδ T cell expressing TRGV9.
322. The bispecific antibody of any one of embodiments 263 to 271 or 287 to 289, wherein the bispecific antibody induces γδ T cell dependent cytotoxicity of a cell expressing TRBC1 in vitro with an EC₅₀ of less than about 160 pM.
323. The bispecific antibody of any one of embodiments 263 to 271 or 287 to 289, wherein the EC₅₀ is assessed with a mixture of γδ T effector cells and target cells expressing the TRBC1 antigen.
324. The bispecific antibody of embodiment 323, wherein the effector cell to target cell ratio is about 0.01 to 1 to about 10 to 1.
325. The bispecific antibody of embodiment 323, wherein the effector cell to target cell ratio is about 0.01 to 1 to about 5 to 1.
326. The bispecific antibody of embodiment 323, wherein the effector cell to target cell ratio is about 0.1 to 1 to about 2 to 1.
327. The bispecific antibody of embodiment 323, wherein the effector cell to target cell ratio is about 1:1.
328. The bispecific antibody of any one of embodiments 263 to 271 or 287 to 289, wherein the bispecific antibody is multivalent.
329. The bispecific antibody of any one of embodiments 263 to 271 or 287 to 289, wherein the bispecific antibody is capable of binding at least three antigens.
330. The bispecific antibody of embodiment 329, wherein the bispecific antibody is capable of binding at least five antigens.
331. A bispecific antibody comprising: a first means capable of binding TRGV9 on the surface of the γδ T cell; and a second means capable of binding a TRBC1 antigen.
332. The bispecific antibody of embodiment 331, wherein the TRBC1 antigen is on the surface of a αβ T cell.
333. A nucleic acid encoding the bispecific antibody of any one of embodiments 263 to 271 or 287 to 289.
334. A vector comprising the nucleic acid of embodiment 333.
335. A host cell comprising the vector of embodiment 334.
336. A kit comprising the vector of embodiment 335 and packaging for the same.
337. A pharmaceutical composition comprising the bispecific antibody of any one of embodiments 263 to 271 or 287 to 289, and a pharmaceutically acceptable carrier.
338. A method of producing the pharmaceutical composition of embodiment 337, comprising combining the bispecific antibody with a pharmaceutically acceptable carrier to obtain the pharmaceutical composition.
339. A process for making an antibody that binds to more than one target molecule, the process comprising: a step for performing a function of obtaining a binding domain capable of binding to a TRGV9 antigen on a γδ T cell; a step for performing a function of obtaining a binding domain capable of binding to a TRBC1 antigen on the surface of an αβ T cell; and a step for performing a function of providing an antibody capable of binding to a TRGV9 antigen on a γδ T cell and a TRBC1 antigen on the surface of a αβ T cell.
340. The process of embodiment 339, wherein the step for performing a function of obtaining a binding domain capable of binding to a TRBC1 antigen on the surface of an αβ T cell is repeated n times and further comprising n steps for performing a function of providing a binding domain capable of binding to a TRGV9 antigen on a γδ T cell and n number of target molecules, wherein n is at least 2.
341. A method of directing a γδ T cell expressing TRGV9 to an αβ T cell expressing TRBC1, the method comprising contacting the γδ T cell with the bispecific antibody of any one of embodiments 263 to 271 or 287 to 289, wherein the contacting directs the γδ T cell to the αβ T cell expressing TRBC1.
342. A method of inhibiting growth or proliferation of αβ T cells expressing a TRBC1 antigen on the cell surface, the method comprising contacting the αβ T cells with the bispecific antibody of any one of embodiments 263 to 271 or 287 to 289, wherein contacting the αβ T cells with the pharmaceutical composition inhibits growth or proliferation of the αβ T cells.
343. The method of embodiment 342, wherein the αβ T cells are in the presence of a γδ T cell expressing TRGV9 while in contact with the bispecific antibody.
344. A method for eliminating T cells expressing TRBC1, or treating a disorder caused all or in part by αβ T cells expressing TRBC1, in a subject, comprising administering an effective amount of the bispecific antibody of any one of embodiments 263 to 271 or 287 to 289 to the subject.
345. The method of embodiment 344, wherein the disorder is a T cell leukemia.
346. The method of embodiment 344, wherein the disorder is a T cell lymphoma.
347. The method of embodiment 344, wherein the disorder is an T cell acute lymphoblastic leukemia (T-ALL).
348. The method of any one of embodiments 344 to 347, wherein the subject is a subject in need thereof.
349. The method of any one of embodiments 344 to 348, wherein the subject is a human.
350. A method of activating a γδ T cell expressing TRGV9, comprising contacting the γδ T cell with the bispecific antibody of any one of embodiments 263 to 271 or 287 to 289.
351. The method of embodiment 350, wherein the contacting results in an increase in CD69, CD25, and/or Granzyme B expression, as compared to a control γδ T cell expressing TRGV9.
352. The bispecific antibody of any one of embodiments 272 to 280 or 290 to 292, wherein the bispecific antibody induces γδ T cell dependent cytotoxicity of a cell expressing BCMA in vitro with an EC₅₀ of less than about 160 pM.
353. The bispecific antibody of antibody of any one of embodiments 272 to 280 or 290 to 292, wherein the EC₅₀ is assessed with a mixture of γδ T effector cells and target cells expressing the BCMA antigen.
354. The bispecific antibody of embodiment 353, wherein the effector cell to target cell ratio is about 0.01 to 1 to about 10 to 1.
355. The bispecific antibody of embodiment 353, wherein the effector cell to target cell ratio is about 0.01 to 1 to about 5 to 1.
356. The bispecific antibody of embodiment 353, wherein the effector cell to target cell ratio is about 0.1 to 1 to about 2 to 1.
357. The bispecific antibody of embodiment 353, wherein the effector cell to target cell ratio is about 1:1.
358. The bispecific antibody of any one of embodiments 272 to 280 or 290 to 292, wherein the bispecific antibody is multivalent.
359. The bispecific antibody of any one of embodiments 272 to 280 or 290 to 292, wherein the bispecific antibody is capable of binding at least three antigens.
360. The bispecific antibody of embodiment 359, wherein the bispecific antibody is capable of binding at least five antigens.
361. A bispecific antibody comprising: a first means capable of binding TRGV9 on the surface of the γδ T cell; and a second means capable of binding a BCMA antigen.
362. The bispecific antibody of embodiment 261, wherein the BCMA antigen is on the surface of a cell.
363. A nucleic acid encoding the bispecific antibody of any one of embodiments antibody of any one of embodiments 272 to 280 or 290 to 292.
364. A vector comprising the nucleic acid of embodiment 363.
365. A host cell comprising the vector of embodiment 364.
366. A kit comprising the vector of embodiment 365 and packaging for the same.
367. A pharmaceutical composition comprising the bispecific antibody of any one of embodiments antibody of any one of embodiments 272 to 280 or 290 to 292, and a pharmaceutically acceptable carrier.
368. A method of producing the pharmaceutical composition of embodiment 367, comprising combining the bispecific antibody with a pharmaceutically acceptable carrier to obtain the pharmaceutical composition.
369. A process for making an antibody that binds to more than one target molecule, the process comprising: a step for performing a function of obtaining a binding domain capable of binding to a TRGV9 antigen on a γδ T cell; a step for performing a function of obtaining a binding domain capable of binding to a BCMA antigen on the surface of a second cell; and a step for performing a function of providing an antibody capable of binding to a TRGV9 antigen on a γδ T cell and a BCMA antigen on the surface of a second cell.
370. The process of embodiment 369, wherein the step for performing a function of obtaining a binding domain capable of binding to a BCMA antigen on the surface of a second cell is repeated n times and further comprising n steps for performing a function of providing a binding domain capable of binding to a TRGV9 antigen on a γδ T cell and n number of target molecules, wherein n is at least 2.
371. A method of directing a γδ T cell expressing TRGV9 to a second cell expressing BCMA, the method comprising contacting the γδ T cell with the bispecific antibody of any one of embodiments 272 to 280 or 290 to 292, wherein the contacting directs the γδ T cell to the cell expressing BCMA.
372. A method of inhibiting growth or proliferation of cells expressing a BCMA antigen on the cell surface, the method comprising contacting the cells expressing BCMA with the bispecific antibody of any one of embodiments 272 to 280 or 290 to 292, wherein contacting the cells expressing BCMA with the pharmaceutical composition inhibits growth or proliferation of the cells expressing the BCMA antigen.
373. The method of embodiment 313, wherein the cells expressing the BCMA antigen are in the presence of a γδ T cell expressing TRGV9 while in contact with the bispecific antibody.
374. A method for eliminating cells expressing BCMA, or treating a disorder caused all or in part by cells expressing BCMA, in a subject, comprising administering an effective amount of the bispecific antibody of any one of embodiments 272 to 280 or 290 to 292 to the subject.
375. The method of embodiment 374, wherein the disorder is a leukemia.
376. The method of embodiment 374, wherein the disorder is a lymphoma.
377. The method of any one of embodiments 374 to 376, wherein the subject is a subject in need thereof.
378. The method of any one of embodiments 374 to 377, wherein the subject is a human.
379. A method of activating a γδ T cell expressing TRGV9, comprising contacting the γδ T cell with the bispecific antibody of any one of embodiments 272 to 280 or 290 to 292.
380. The method of embodiment 379, wherein the contacting results in an increase in CD69, CD25, and/or Granzyme B expression, as compared to a control γδ T cell expressing TRGV9.
381. An antibody that binds to TRGV9, comprising: a HC1 comprising a HCDR1, HCDR2, and HCDR3 comprising the amino acid sequences of SEQ ID NO:1, SEQ ID NO:2, and SEQ ID NO:3, respectively, and a LC1 comprising a LCDR1, LCDR2, and LCDR3 comprising the amino acid sequences of SEQ ID NO:4, SEQ ID NO:5, and SEQ ID NO:6, respectively, to form a binding site for the TRGV9.
382. An antibody that binds to TRGV9, comprising: a HC1 comprising a HCDR1, HCDR2, and HCDR3 comprising the amino acid sequences of SEQ ID NO:1, SEQ ID NO:2, and SEQ ID NO:31, respectively, and a LC1 comprising a LCDR1, LCDR2, and LCDR3 comprising the amino acid sequences of SEQ ID NO:4, SEQ ID NO:5, and SEQ ID NO:6, respectively, to form a binding site for the TRGV9.
383. An antibody that binds to TRGV9, comprising: a HC1 comprising a HCDR1, HCDR2, and HCDR3 comprising the amino acid sequences of SEQ ID NO:1, SEQ ID NO:2, and SEQ ID NO:32, respectively, and a LC1 comprising a LCDR1, LCDR2, and LCDR3 comprising the amino acid sequences of SEQ ID NO:4, SEQ ID NO:5, and SEQ ID NO:6, respectively, to form a binding site for the TRGV9.
384. An antibody that binds to TRGV9, comprising: a HC1 comprising a HCDR1, HCDR2, and HCDR3 comprising the amino acid sequences of SEQ ID NO:1, SEQ ID NO:2, and SEQ ID NO:33, respectively, and a LC1 comprising a LCDR1, LCDR2, and LCDR3 comprising the amino acid sequences of SEQ ID NO:4, SEQ ID NO:5, and SEQ ID NO:6, respectively, to form a binding site for the TRGV9.
385. An antibody that binds to TRGV9, comprising: a HC1 comprising a HCDR1, HCDR2, and HCDR3 comprising the amino acid sequences of SEQ ID NO:1, SEQ ID NO:76, and SEQ ID NO:3, respectively, and a LC1 comprising a LCDR1, LCDR2, and LCDR3 comprising the amino acid sequences of SEQ ID NO:77, SEQ ID NO:5, and SEQ ID NO:6, respectively, to form a binding site for the TRGV9.
386. An antibody that binds to TRGV9, comprising: a HC1 comprising a HCDR1, HCDR2, and HCDR3 comprising the amino acid sequences of SEQ ID NO:60, SEQ ID NO:61, and SEQ ID NO:62, respectively, and a LC1 comprising a LCDR1, LCDR2, and LCDR3 comprising the amino acid sequences of SEQ ID NO:63, SEQ ID NO:64, and SEQ ID NO:6, respectively, to form a binding site for the TRGV9.
387. An antibody that binds to TRGV9, comprising: a HC1 comprising a HCDR1, HCDR2, and HCDR3 comprising the amino acid sequences of SEQ ID NO:89, SEQ ID NO:90, and SEQ ID NO:91, respectively, and a LC1 comprising a LCDR1, LCDR2, and LCDR3 comprising the amino acid sequences of SEQ ID NO:92, SEQ ID NO:93, and SEQ ID NO:94, respectively, to form a binding site for the TRGV9.
388. An antibody that binds to TRGV9, comprising: a HC1 comprising a HCDR1, HCDR2, and HCDR3 comprising the amino acid sequences of SEQ ID NO:98, SEQ ID NO:99, and SEQ ID NO:100, respectively, and a LC1 comprising a LCDR1, LCDR2, and LCDR3 comprising the amino acid sequences of SEQ ID NO:101, SEQ ID NO:102, and SEQ ID NO:103, respectively, to form a binding site for the TRGV9.
389. An antibody that binds to TRGV9, comprising: a HC1 comprising a HCDR1, HCDR2, and HCDR3 comprising the amino acid sequences of SEQ ID NO:107, SEQ ID NO:108, and SEQ ID NO:109, respectively, and a LC1 comprising a LCDR1, LCDR2, and LCDR3 comprising the amino acid sequences of SEQ ID NO:110, SEQ ID NO:111, and SEQ ID NO:112, respectively, to form a binding site for the TRGV9.
390. An antibody that binds to TRGV9, comprising: a HC1 comprising a HCDR1, HCDR2, and HCDR3 comprising the amino acid sequences of SEQ ID NO:117, SEQ ID NO:118, and SEQ ID NO:119, respectively, and a LC1 comprising a LCDR1, LCDR2, and LCDR3 comprising the amino acid sequences of SEQ ID NO:120, SEQ ID NO:121, and SEQ ID NO:122, respectively, to form a binding site for the TRGV9.
391. An antibody that binds to TRGV9, comprising a HC1 comprising a HCDR1, HCDR2, and HCDR3 comprising the amino acid sequences of SEQ ID NO:127, SEQ ID NO:128, and SEQ ID NO:129, respectively, and a LC1 comprising a LCDR1, LCDR2, and LCDR3 comprising the amino acid sequences of SEQ ID NO:130, SEQ ID NO:131, and SEQ ID NO:132, respectively, to form a binding site for the TRGV9.
392. An antibody that binds to TRGV9, comprising a VH having an amino acid sequence of SEQ ID NO:7.
393. An antibody that binds to TRGV9, comprising a VL having an amino acid sequence of SEQ ID NO:8.
394. An antibody that binds to TRGV9, comprising a VH having an amino acid sequence of SEQ ID NO:7, and a VL having an amino acid sequence of SEQ ID NO:8.
395. An antibody that binds to TRGV9, comprising a VH having an amino acid sequence of SEQ ID NO:34.
396. An antibody that binds to TRGV9, comprising a VL having an amino acid sequence of SEQ ID NO:8.
397. An antibody that binds to TRGV9, comprising a VH having an amino acid sequence of SEQ ID NO:34, and a VL having an amino acid sequence of SEQ ID NO:8.
398. An antibody that binds to TRGV9, comprising a VH having an amino acid sequence of SEQ ID NO:35.
399. An antibody that binds to TRGV9, comprising a VL having an amino acid sequence of SEQ ID NO:8.
400. An antibody that binds to TRGV9, comprising a VH having an amino acid sequence of SEQ ID NO:35, and a VL having an amino acid sequence of SEQ ID NO:8.
401. An antibody that binds to TRGV9, comprising a VH having an amino acid sequence of SEQ ID NO:36.
402. An antibody that binds to TRGV9, comprising a VL having an amino acid sequence of SEQ ID NO:8.
403. An antibody that binds to TRGV9, comprising a VH having an amino acid sequence of SEQ ID NO:36, and a VL having an amino acid sequence of SEQ ID NO:8.
404. An antibody that binds to TRGV9, comprising a VH having an amino acid sequence of SEQ ID NO:65.
405. An antibody that binds to TRGV9, comprising a VL having an amino acid sequence of SEQ ID NO:66.
406. An antibody that binds to TRGV9, comprising a VH having an amino acid sequence of SEQ ID NO:65, and a VL having an amino acid sequence of SEQ ID NO:66.
407. An antibody that binds to TRGV9, comprising a VH having an amino acid sequence of SEQ ID NO:67.
408. An antibody that binds to TRGV9, comprising a VL having an amino acid sequence of SEQ ID NO:68.
409. An antibody that binds to TRGV9, comprising a VH having an amino acid sequence of SEQ ID NO:67, and a VL having an amino acid sequence of SEQ ID NO:68.
410. An antibody that binds to TRGV9, comprising a VH having an amino acid sequence of SEQ ID NO:95.
411. An antibody that binds to TRGV9, comprising a VL having an amino acid sequence of SEQ ID NO:96.
412. An antibody that binds to TRGV9, comprising a VH having an amino acid sequence of SEQ ID NO:95, and a VL having an amino acid sequence of SEQ ID NO:96.
413. An antibody that binds to TRGV9, comprising a VH having an amino acid sequence of SEQ ID NO:104.
414. An antibody that binds to TRGV9, comprising a VL having an amino acid sequence of SEQ ID NO:105.
415. An antibody that binds to TRGV9, comprising a VH having an amino acid sequence of SEQ ID NO:104, and a VL having an amino acid sequence of SEQ ID NO:105.
416. An antibody that binds to TRGV9, comprising a VH having an amino acid sequence of SEQ ID NO:113.
417. An antibody that binds to TRGV9, comprising a VL having an amino acid sequence of SEQ ID NO:114.
418. An antibody that binds to TRGV9, comprising a VH having an amino acid sequence of SEQ ID NO:113, and a VL having an amino acid sequence of SEQ ID NO:114.
419. An antibody that binds to TRGV9, comprising a VH having an amino acid sequence of SEQ ID NO:123.
420. An antibody that binds to TRGV9, comprising a VL having an amino acid sequence of SEQ ID NO:124.
421. An antibody that binds to TRGV9, comprising a VH having an amino acid sequence of SEQ ID NO:123, and a VL having an amino acid sequence of SEQ ID NO:124.
422. An antibody that binds to TRGV9, comprising a VH having an amino acid sequence of SEQ ID NO:133.
423. An antibody that binds to TRGV9, comprising a VL having an amino acid sequence of SEQ ID NO:134.
424. An antibody that binds to TRGV9, comprising a VH having an amino acid sequence of SEQ ID NO:133, and a VL having an amino acid sequence of SEQ ID NO:134.
425. An antibody that binds to TRGV9, comprising a heavy chain having an amino acid sequence of SEQ ID NO:69.
426. An antibody that binds to TRGV9, comprising a light chain having an amino acid sequence of SEQ ID NO:24.
427. An antibody that binds to TRGV9, comprising a heavy chain having an amino acid sequence of SEQ ID NO:69, and a light chain having an amino acid sequence of SEQ ID NO:24.
428. An antibody that binds to TRGV9, comprising a heavy chain having an amino acid sequence of SEQ ID NO:71.
429. An antibody that binds to TRGV9, comprising a light chain having an amino acid sequence of SEQ ID NO:72.
430. An antibody that binds to TRGV9, comprising a heavy chain having an amino acid sequence of SEQ ID NO:71, and a light chain having an amino acid sequence of SEQ ID NO:72.
431. An antibody that binds to TRGV9, comprising a heavy chain having an amino acid sequence of SEQ ID NO:74.
432. An antibody that binds to TRGV9, comprising a light chain having an amino acid sequence of SEQ ID NO:75.
433. An antibody that binds to TRGV9, comprising a heavy chain having an amino acid sequence of SEQ ID NO:74, and a light chain having an amino acid sequence of SEQ ID NO:75.
434. An antibody that binds to TRGV9, comprising a heavy chain having an amino acid sequence of SEQ ID NO:115.
435. An antibody that binds to TRGV9, comprising a light chain having an amino acid sequence of SEQ ID NO:116.
436. An antibody that binds to TRGV9, comprising a heavy chain having an amino acid sequence of SEQ ID NO:115, and a light chain having an amino acid sequence of SEQ ID NO:116.
437. An antibody that binds to TRGV9, comprising a heavy chain having an amino acid sequence of SEQ ID NO:125.
438. An antibody that binds to TRGV9, comprising a light chain having an amino acid sequence of SEQ ID NO:126.
439. An antibody that binds to TRGV9, comprising a heavy chain having an amino acid sequence of SEQ ID NO:125, and a light chain having an amino acid sequence of SEQ ID NO:126.
440. An antibody that binds to TRGV9, comprising a heavy chain having an amino acid sequence of SEQ ID NO:135.
441. An antibody that binds to TRGV9, comprising a light chain having an amino acid sequence of SEQ ID NO:136.
442. An antibody that binds to TRGV9, comprising a heavy chain having an amino acid sequence of SEQ ID NO:135, and a light chain having an amino acid sequence of SEQ ID NO:136.
443. The antibody of any one of 381 to 442, wherein the antibody is a multispecific antibody.
444. The multispecific antibody of embodiment 443, wherein the antibody further binds CD123.
445. The multispecific antibody of embodiment 443, wherein the antibody further binds CD33.
446. The multispecific antibody of embodiment 443, wherein the antibody further binds TRBC1.
447. The multispecific antibody of embodiment 443, wherein the antibody further binds BCMA.
448. The multispecific antibody of embodiment 443, wherein the antibody further binds PSMA.
449. The antibody of any one of 381 to 442, wherein the antibody is a bispecific antibody.
450. The bispecific antibody of embodiment 449, wherein the antibody further binds CD123.
451. The bispecific antibody of embodiment 449, wherein the antibody further binds CD33.
452. The bispecific antibody of embodiment 449, wherein the antibody further binds TRBC1.
453. The bispecific antibody of embodiment 449, wherein the antibody further binds BCMA.
454. The bispecific antibody of embodiment 449, wherein the antibody further binds PSMA.

Provided in the Examples herein are exemplary multi-specific (bispecific) antibodies that bind to TRGV9 and CD123 (also known as IL3RA). CD123 is expressed on a variety of cell types in various tissues, including adipose tissue, adrenal gland, appendix, bone marrow, breast, bronchus, caudate, cerebellum, cerebral cortex, cervix, uterine, colon, duodenum, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine, smooth muscle, soft tissue, spleen, stomach, testis, thyroid gland, tonsil, urinary bladder, and vagina (see, e.g., proteinatlas.org). Thus, these Examples are illustrative of exemplary bispecific antibodies that can effectively target a wide variety of cells and tissues in a subject.

In some embodiments, provided herein is a bispecific antibody comprising: (a) a first binding domain that binds to a TRGV9 antigen, and (b) a second binding domain that binds to a second target antigen. In some embodiments, provided herein is a bispecific antibody comprising: (a) a first binding domain that specifically binds to a TRGV9 antigen, and (b) a second binding domain that specifically binds to a second target antigen. In some embodiments, provided herein is a bispecific antibody comprising: (a) a first binding domain that binds to a first epitope on a TRGV9 antigen, and (b) a second binding domain that binds to a second epitope on a second target antigen. In some embodiments, provided herein is a bispecific antibody comprising: (a) a first binding domain that specifically binds to a first epitope on a TRGV9 antigen, and (b) a second binding domain that specifically binds to a second epitope on a second target antigen. In certain embodiments, the second target antigen is CD123.

Exemplary binding agents that bind to TRGV9, as well as exemplary binding agents that bind to CD123 are provided elsewhere herein, for example in the Sequence Listing, Examples, as well as Table 1.1, Table 1.2, and Tables 1-40.

Also provided in the Examples herein are exemplary multi-specific (bispecific) antibodies that bind to TRGV9 and CD33. These Examples are illustrative of additional exemplary bispecific antibodies that can effectively target a variety of cells and tissues in a subject. In some embodiments, provided herein is a bispecific antibody comprising: (a) a first binding domain that binds to a TRGV9 antigen, and (b) a second binding domain that binds to a second target antigen. In some embodiments, provided herein is a bispecific antibody comprising: (a) a first binding domain that specifically binds to a TRGV9 antigen, and (b) a second binding domain that specifically binds to a second target antigen. In some embodiments, provided herein is a bispecific antibody comprising: (a) a first binding domain that binds to a first epitope on a TRGV9 antigen, and (b) a second binding domain that binds to a second epitope on a second target antigen. In some embodiments, provided herein is a bispecific antibody comprising: (a) a first binding domain that specifically binds to a first epitope on a TRGV9 antigen, and (b) a second binding domain that specifically binds to a second epitope on a second target antigen. In certain embodiments, the second target antigen is CD33.

Exemplary binding agents that bind to TRGV9, as well as exemplary binding agents that bind to CD33 are provided elsewhere herein, for example in the Sequence Listing, Examples, as well as Table 1.1, Table 1.2, and Tables 1-40.

Also provided in the Examples herein are exemplary multi-specific (bispecific) antibodies that bind to TRGV9 and TRBC1. These Examples are illustrative of additional exemplary bispecific antibodies that can effectively target a variety of cells and tissues in a subject. In some embodiments, provided herein is a bispecific antibody comprising: (a) a first binding domain that binds to a TRGV9 antigen, and (b) a second binding domain that binds to a second target antigen. In some embodiments, provided herein is a bispecific antibody comprising: (a) a first binding domain that specifically binds to a TRGV9 antigen, and (b) a second binding domain that specifically binds to a second target antigen. In some embodiments, provided herein is a bispecific antibody comprising: (a) a first binding domain that binds to a first epitope on a TRGV9 antigen, and (b) a second binding domain that binds to a second epitope on a second target antigen. In some embodiments, provided herein is a bispecific antibody comprising: (a) a first binding domain that specifically binds to a first epitope on a TRGV9 antigen, and (b) a second binding domain that specifically binds to a second epitope on a second target antigen. In certain embodiments, the second target antigen is TRBC1.

Exemplary binding agents that bind to TRGV9, as well as exemplary binding agents that bind to TRBC1 are provided elsewhere herein, for example in the Sequence Listing, Examples, as well as Table 1.1, Table 1.2, and Tables 1-40.

Also provided in the Examples herein are exemplary multi-specific (bispecific) antibodies that bind to TRGV9 and BCMA. These Examples are illustrative of additional exemplary bispecific antibodies that can effectively target a variety of cells and tissues in a subject. In some embodiments, provided herein is a bispecific antibody comprising: (a) a first binding domain that binds to a TRGV9 antigen, and (b) a second binding domain that binds to a second target antigen. In some embodiments, provided herein is a bispecific antibody comprising: (a) a first binding domain that specifically binds to a TRGV9 antigen, and (b) a second binding domain that specifically binds to a second target antigen. In some embodiments, provided herein is a bispecific antibody comprising: (a) a first binding domain that binds to a first epitope on a TRGV9 antigen, and (b) a second binding domain that binds to a second epitope on a second target antigen. In some embodiments, provided herein is a bispecific antibody comprising: (a) a first binding domain that specifically binds to a first epitope on a TRGV9 antigen, and (b) a second binding domain that specifically binds to a second epitope on a second target antigen. In certain embodiments, the second target antigen is BCMA.

Exemplary binding agents that bind to TRGV9, as well as exemplary binding agents that bind to BCMA are provided elsewhere herein, for example in the Sequence Listing, Examples, as well as Table 1.1, Table 1.2, and Tables 1-40.

Also provided in the Examples herein are exemplary multi-specific (bispecific) antibodies that bind to TRGV9 and PSMA. These Examples are illustrative of additional exemplary bispecific antibodies that can effectively target a variety of cells and tissues in a subject. In some embodiments, provided herein is a bispecific antibody comprising: (a) a first binding domain that binds to a TRGV9 antigen, and (b) a second binding domain that binds to a second target antigen. In some embodiments, provided herein is a bispecific antibody comprising: (a) a first binding domain that specifically binds to a TRGV9 antigen, and (b) a second binding domain that specifically binds to a second target antigen. In some embodiments, provided herein is a bispecific antibody comprising: (a) a first binding domain that binds to a first epitope on a TRGV9 antigen, and (b) a second binding domain that binds to a second epitope on a second target antigen. In some embodiments, provided herein is a bispecific antibody comprising: (a) a first binding domain that specifically binds to a first epitope on a TRGV9 antigen, and (b) a second binding domain that specifically binds to a second epitope on a second target antigen. In certain embodiments, the second target antigen is PSMA.

Exemplary binding agents that bind to TRGV9, as well as exemplary binding agents that bind to PSMA are provided elsewhere herein, for example in the Sequence Listing, Examples, as well as Table 1.1, Table 1.2, and Tables 1-40.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### EXAMPLES

### Example 1: Production of Multispecific Antibodies that Bind γδ T Cells

1.1: Production of Mabs that Bind γδ T Cell Antigens

Antigens or portions of antigens specific for γδ T cells are used to immunize an animal (e.g., a mouse or a rabbit). To generate the γδ T cell monoclonal antibodies, peripheral blood mononuclear cells are isolated from the whole blood of the immunized animal, and antigen specific B cells are grown. B cells secreting reactive antibodies for the γδ T cell antigens are identified by an antigen-binding ELISA screening of the B cell culture supernatants. High binding ELISA plates are coated with the γδ T cell antigen overnight. The ELISA plates are blocked, and diluted B cell culture supernatants are added to the plates. The plates are incubated at room temperature and following incubation, a secondary antibody specific for recognizing the γδ T cell antigen antibody is added to the plate to determine if the γδ T cell antigen antibody bound the γδ T cell antigen. Binding of the antibody is determined by reaction of a substrate on the secondary antibody.

After the identification of monoclonal antibodies that are capable of binding γδ T cell antigens, the variable regions of the heavy and light chains of the γδ T cell antibody are sequenced. Constructs are created for the expression of the heavy and light chain of the γδ T cell antibody. The constructs are transfected into a host cell to express the heavy and light chains, and the γδ T cell antibody is isolated from the supernatant.

1.2: Production of γδ T Cell Bispecific Antibodies

The variable region sequence of the γδ T cell monoclonal antibody and a second monoclonal antibody capable of binding a target antigen on a target cell of interest are used to generate a bispecific antibody to be tested for γδ T cell re-directed killing of the target cells. Target antigens of interest can be selected from, but not limited, antigens described in Zhang et al., Nucleic Acids Research 47(D1):D721-D728 (2019). γδ T cell bispecific antibodies are produced as full-length antibodies in the knob-into-hole format as human IgG4, as previously described (Atwell et al., J. Mol. Biol. 270:26-35 (1997)). Nucleic acid sequences encoding variable regions are sub-cloned into custom mammalian expression vectors containing the constant region of IgG4 expression cassettes using standard PCR restriction enzyme based cloning techniques. The bispecific antibodies are expressed by transient transfection in Chinese hamster ovary cell line. The antibodies are initially purified by MAB SELECT SURE Protein A column (GE Healthcare, Piscataway, N.J.) (Brown, Bottomley et al. Biochem Soc Trans. 1998 August; 26(3):S249.). The column is equilibrated with Phosphate Buffer Saline (PBS), pH 7.2 and is loaded with fermentation supernatant at a flow rate of 2 mL/min. After loading, the column is washed with PBS (4 column volumes (CV)) followed by elution in 30 mM sodium acetate, pH 3.5. Fractions containing protein peaks as monitored by absorbance at 280 nm in Akta Explorer (GE healthcare) are pooled together and are neutralized to pH 5.0 by adding 1% of 3M sodium acetate, pH 9.0. As a polishing step, the antibodies are purified on a preparative size exclusion chromatography (SEC) using a SUPERDEX 200 column (GE healthcare). The integrity of sample is assessed by endotoxin measurement and SDS polyacrylamide gel electrophoresis under reducing and non-reducing conditions. The final protein concentrations are determined.

1.3: Production of Anti-TRGV9 Bispecific Antibodies

Variable region sequences of an exemplary anti-TRGV9 monoclonal antibody are provided below in Table 1.1 and Table 1.2.

TABLE 1.1
Anti-TRGV9 mAb CDR Sequences

| Antibody | VH CDR1 | VH CDR2 | VH CDR3 | VL CDR1 | VL CDR2 | VL CDR3 |
|---|---|---|---|---|---|---|
| TRGV9Ab_1 | DHYIN | QIYPGDGNTYYN | NYGDYTIDF | KSSQSLLYSSNQKNYLA | WASTRES | QQYYRYHT |
| | (SEQ ID | QKFKG | (SEQ ID NO: | (SEQ ID NO: 4) | (SEQ ID NO: | (SEQ ID NO: |
| | NO: 1) | (SEQ ID NO: 2) | 3) | | 5) | 6) |
| TRGV9Ab_2 | DHYIN | QIYPGDGNTYYN | NMGMYTIDF | KSSQSLLYSSNQKNYLA | WASTRES | QQYYRYHT |
| | (SEQ ID | QKFKG | (SEQ ID | (SEQ ID NO: 4) | (SEQ ID NO: | (SEQ ID NO: |
| | NO: 1) | (SEQ ID NO: 2) | NO: 31) | | 5) | 6) |
| TRGV9Ab_3 | DHYIN | QIYPGDGNTYYN | NMGMYTLDF | KSSQSLLYSSNQKNYLA | WASTRES | QQYYRYHT |
| | (SEQ ID | QKFKG | (SEQ ID | (SEQ ID NO: 4) | (SEQ ID NO: | (SEQ ID NO: |
| | NO: 1) | (SEQ ID NO: 2) | NO: 32) | | 5) | 6) |
| TRGV9Ab_4 | DHYIN | QIYPGDGNTYYN | NYGDYTLDF | KSSQSLLYSSNQKNYLA | WASTRES | QQYYRYHT |
| | (SEQ ID | QKFKG | (SEQ ID | (SEQ ID NO: 4) | (SEQ ID NO: | (SEQ ID NO: |
| | NO: 1) | (SEQ ID NO: 2) | NO: 33) | | 5) | 6) |

TABLE 1.2
Anti-TRGV9 mAb VH and VL Domain Sequences

| Antibody | VH | VL |
|---|---|---|
| TRGV9Ab_1 | EVQLQQSGAELARPGASVKLSCKASGFTFT | DIVMSQSPSSLAVSVGEKVTMSCKSSQSLL |
| | DHYINWVKQRTGQGLEWIGQIYPGDGNTYY | YSSNQKNYLAWYQQKPGQSPKLLIYWASTR |
| | NQKFKGKATLTADKSSSTAYMQLSSLTSED | ESGVPDRFTGSGSGTDFTLTISSVKAEDLA |
| | SAVYFCAPNYGDYTIDFWGQGTSVTVSS | VYYCQQYYRYHTFGTGTKLEIK |
| | (SEQ ID NO: 7) | (SEQ ID NO: 8) |
| TRGV9Ab_2 | EVQLQQSGAELARPGASVKLSCKASGFTFT | DIVMSQSPSSLAVSVGEKVTMSCKSSQSLL |
| | DHYINWVKQRTGQGLEWIGQIYPGDGNTYY | YSSNQKNYLAWYQQKPGQSPKLLIYWASTR |
| | NQKFKGKATLTADKSSSTAYMQLSSLTSED | ESGVPDRFTGSGSGTDFTLTISSVKAEDLA |
| | SAVYFCAPNMGMYTIDFWGQGTSVTVSS | VYYCQQYYRYHTFGTGTKLEIK |
| | (SEQ ID NO: 34) | (SEQ ID NO: 8) |
| TRGV9Ab_3 | EVQLQQSGAELARPGASVKLSCKASGFTFT | DIVMSQSPSSLAVSVGEKVTMSCKSSQSLL |
| | DHYINWVKQRTGQGLEWIGQIYPGDGNTYY | YSSNQKNYLAWYQQKPGQSPKLLIYWASTR |
| | NQKFKGKATLTADKSSSTAYMQLSSLTSED | ESGVPDRFTGSGSGTDFTLTISSVKAEDLA |
| | SAVYFCAPNMGMYTLDFWGQGTSVTVSS | VYYCQQYYRYHTFGTGTKLEIK |
| | (SEQ ID NO: 35) | (SEQ ID NO: 8) |
| TRGV9Ab_4 | EVQLQQSGAELARPGASVKLSCKASGFTFT | DIVMSQSPSSLAVSVGEKVTMSCKSSQSLL |
| | DHYINWVKQRTGQGLEWIGQIYPGDGNTYY | YSSNQKNYLAWYQQKPGQSPKLLIYWASTR |
| | NQKFKGKATLTADKSSSTAYMQLSSLTSED | ESGVPDRFTGSGSGTDFTLTISSVKAEDLA |
| | SAVYFCAPNYGDYTLDFWGQGTSVTVSS | VYYCQQYYRYHTFGTGTKLEIK |
| | (SEQ ID NO: 36) | (SEQ ID NO: 8) |

Variable region sequences of a TRGV9 monoclonal antibody and a second monoclonal antibody capable of binding a target antigen on a target cell of interest are used to generate a bispecific antibody to be tested for γδ T cell re-directed killing of the target cells. Target antigens of interest can be selected from, but not limited to, antigens described in Zhang et al., Nucleic Acids Research 47(D1):D721-D728 (2019). Anti-TRGV9 bispecific antibodies are produced as full-length antibodies in the knob-into-hole format as human IgG4, as previously described (Atwell et al., J. Mol. Biol. 270:26-35 (1997)). Nucleic acid sequences encoding variable regions are sub-cloned into custom mammalian expression vectors containing the constant region of IgG4 expression cassettes using standard PCR restriction enzyme based cloning techniques. The bispecific antibodies are expressed by transient transfection in Chinese hamster ovary cell line. The antibodies are initially purified by Mab Select SuRe Protein A column (GE Healthcare, Piscataway, N.J.) (Brown, Bottomley et al. Biochem Soc Trans. 1998 August; 26(3):5249). The column is equilibrated with Phosphate Buffer Saline (PBS), pH 7.2 and is loaded with fermentation supernatant at a flow rate of 2 mL/min. After loading, the column is washed with PBS (4 column volumes (CV)) followed by elution in 30 mM sodium acetate, pH 3.5. Fractions containing protein peaks as monitored by Absorbance at 280 nm in Akta Explorer (GE healthcare) are pooled together and are neutralized to pH 5.0 by adding 1% of 3M sodium acetate, pH 9.0. As a polishing step, the antibodies are purified on a preparative size exclusion chromatography (SEC) using a SUPERDEX 200 column (GE healthcare). The integrity of sample is assessed by endotoxin measurement and SDS polyacrylamide gel electrophoresis under reducing and non-reducing conditions. The final protein concentrations are determined.

### Example 2—Multispecific Antibodies that Bind TRGV9 and A Cancer Antigen

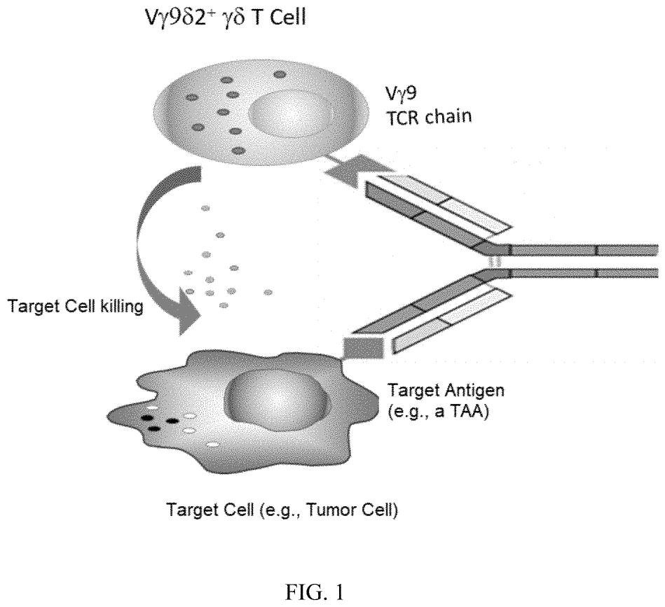

Examples 2.1-2.4 are based on the premise that γδ T cells, which mainly express heterodimers of TRGV9 and Vδ2 chains demonstrate potent anti-tumor functions. These cells express TCR-TRGV9 and the majority, if not all, of these cells exhibit efficient cytotoxicity of tumor target cells. This ability is then harnessed using bispecific antibodies constructed such that one arm binds to the TRGV9 structure and the other arm binds to a tumor-associated antigen expressed by the tumor cells. Thus, the bispecific antibody bridges the effector and target cells together-resulting in tumor cell killing. This mechanism of action is described in the schematic outlined in FIG. 1.

The subsequent examples can be divided into the following categories: (1) Generation and characterization of bispecific antibodies capable of binding to the TRGV9 arm expressed on γδ T cells and a cancer antigen (e.g., CD123) on cancer cells (Examples 2.1, 2.2, and 2.3); and (2) Evidence for bispecific antibody-enabled target cell killing by γδ T cells expanded in vitro (Example 2.4).

### Example 2.1: Production of Anti-TRGV9 Mab

The mouse IgG1 anti-human T cell receptor TRGV9 clone 7A5 was sourced commercially. Sample preparation and LC/MSMS analysis were performed by Lake Pharma. (San Carlos, Calif.). The sample was reduced and alkylated, divided into seven aliquots, and proteolytically digested with Trypsin/LysC, Chymotrypsin, LysC, Pepsin, and AspN, Elastase, and Proteinase K enzymes. Resulting peptides were desalted using a ZipTip C18 Pipette Tips and separated on-line using reverse phase chromatography. Mass spectrometry was performed on Thermo Q-Exactive spectrometer using HCD fragmentation. MS data sets were analyzed using PEAKS software by matching de novo sequence tags to an IMGT-based antibody sequences database. Gaps in the sequence were assigned using Contig sequence assembly of de novo identified peptides. All CDRs and hyper-mutations were confirmed by inspecting the MS/MS spectra

The sequences obtained are shown in Tables 1-5.

TABLE 1
CDR Sequences of anti-TRGV9 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| LP7A5_1 | DHYIN | 1 | QIYPGDGNT | 2 | NYGDYT | 3 |
| | | | YYNQKFKG | | IDF | |
| | | SEQ | | SEQ | | SEQ |
| | | ID | | ID | | ID |
| Antibody | LCDR1 | NO: | LCDR2 | NO: | LCDR3 | NO: |
| LP7A5_1 | KSSQSLLYS | 4 | WASTRES | 5 | QQYYRY | 6 |
| | SNQKNYLA | | | | HT | |

TABLE 2
CDR Sequences of anti-TRGV9 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| LP7A5_2 | DHYIN | 1 | QIYPGDGNT | 2 | NMGMYTI | 31 |
| | | | YYNQKFKG | | DF | |
| | | SEQ | | SEQ | | SEQ |
| | | ID | | ID | | ID |
| Antibody | LCDR1 | NO: | LCDR2 | NO: | LCDR3 | NO: |
| LP7A5_2 | KSSQSLLYS | 4 | WASTRES | 5 | QQYYRYH | 6 |
| | SNQKNYLA | | | | T | |

TABLE 3
CDR Sequences of anti-TRGV9 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| LP7A5_3 | DHYIN | 1 | QIYPGDGNT | 2 | NMGMYTL | 32 |
| | | | YYNQKFKG | | DF | |
| | | SEQ | | SEQ | | SEQ |
| | | ID | | ID | | ID |
| Antibody | LCDR1 | NO: | LCDR2 | NO: | LCDR3 | NO: |
| LP7A5_3 | KSSQSLLYS | 4 | WASTRES | 5 | QQYYRYH | 6 |
| | SNQKNYLA | | | | T | |

TABLE 4
CDR Sequences of anti-TRGV9 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| LP7A5_4 | DHYIN | 1 | QIYPGDGNT | 2 | NYGDYTL | 33 |
| | | | YYNQKFKG | | DF | |
| | | SEQ | | SEQ | | SEQ |
| | | ID | | ID | | ID |
| Antibody | LCDR1 | NO: | LCDR2 | NO: | LCDR3 | NO: |
| LP7A5_4 | KSSQSLLYS | 4 | WASTRES | 5 | QQYYRYH | 6 |
| | SNQKNYLA | | | | T | |

TABLE 5
Heavy chain and light chain sequences of
anti-TRGV9 mAb.

| | | SEQ |
|---|---|---|
| | | ID |
| mAb | Heavy Chain Amino Acid Sequence | NO: |
| LP7A5_1 | EVQLQQSGAELARPGASVKLSCKASG | 7 |
| | FTFTDHYINWVKQRTGQGLEWIGQIY | |
| | PGDGNTYYNQKFKGKATLTADKSSST | |
| | AYMQLSSLTSEDSAVYFCAPNYGDYT | |
| | IDFWGQGTSVTVSS | |
| LP7A5_2 | EVQLQQSGAELARPGASVKLSCKASG | 34 |
| | FTFTDHYINWVKQRTGQGLEWIGQIY | |
| | PGDGNTYYNQKFKGKATLTADKSSST | |
| | AYMQLSSLTSEDSAVYFCAPNMGMYT | |
| | IDFWGQGTSVTVSS | |
| LP7A5_3 | EVQLQQSGAELARPGASVKLSCKASG | 35 |
| | FTFTDHYINWVKQRTGQGLEWIGQIY | |
| | PGDGNTYYNQKFKGKATLTADKSSST | |
| | AYMQLSSLTSEDSAVYFCAPNMGMYT | |
| | LDFWGQGTSVTVSS | |
| LP7A5_4 | EVQLQQSGAELARPGASVKLSCKASG | 36 |
| | FTFTDHYINWVKQRTGQGLEWIGQIY | |
| | PGDGNTYYNQKFKGKATLTADKSSST | |
| | AYMQLSSLTSEDSAVYFCAPNYGDYT | |
| | LDFWGQGTSVTVSS | |
| LP7A5_1 | DIVMSQSPSSLAVSVGEKVTMSCKSS | 8 |
| | QSLLYSSNQKNYLAWYQQKPGQSPKL | |
| | LIYWASTRESGVPDRFTGSGSGTDFT | |
| | LTISSVKAEDLAVYYCQQYYRYHTFG | |
| | TGTKLEIK | |

### Example 2.2: Preparation of Anti-TRGV9/Anti-CD123 Bispecific Antibodies

The variable region sequence of LP7A5 (anti-TRGV9) and I3RB217 (anti-CD123 antibody) (HCDRs and LCDRs in Table 6, HC and LC in Table 7) were used to generate a bispecific antibody to be tested for T cell re-directed killing of acute myeloid leukemia (AML) cells. VG1 (anti-TRGV9×CD123) and VG3 (anti-TRGV9×Null) bispecific antibodies were produced as full-length antibodies in the knob-into-hole format as human IgG4, as previously described (Atwell et al. J. Mol. Biol. 270: 26-35, 1997). Nucleic acid sequences encoding variable regions were sub-cloned into a custom mammalian expression vectors containing constant region of IgG4 expression cassettes using standard PCR restriction enzyme based cloning techniques. The bispecific antibodies were expressed by transient transfection in Chinese hamster ovary cell line. The antibodies were initially purified by Mab Select SuRe Protein A column (GE Healthcare, Piscataway, N.J.) (Brown, Bottomley et al. Biochem Soc Trans. 1998 August; 26(3):S249.). The column was equilibrated with Phosphate Buffer Saline (PBS), pH 7.2 and loaded with fermentation supernatant at a flow rate of 2 mL/min. After loading, the column was washed with PBS (4 column volumes (CV)) followed by elution in 30 mM sodium acetate, pH 3.5. Fractions containing protein peaks as monitored by Absorbance at 280 nm in Akta Explorer (GE healthcare) were pooled together and were neutralized to pH 5.0 by adding 1% of 3M sodium acetate, pH 9.0. As a polishing step, the antibodies were purified on a preparative size exclusion chromatography (SEC) using a Superdex 200 column (GE healthcare). The integrity of sample was assessed by endotoxin measurement and SDS polyacrylamide gel electrophoresis under reducing and non-reducing conditions. The final protein concentrations were 1.0 mg/ml for anti-TRGV9/anti-CD123 and 1.0 mg/mL for anti-TRGV9/Null. The final EU levels of anti-TRGV9/anti-CD123 and anti-TRGV9/Null based on these were <3.0 EU/mg.

TABLE 6
CDR Sequences of anti-CD123 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| I3RB217 | SYWIS | 9 | IIDPSDSDT | 10 | GDGSTDL | 11 |
| | | | RYSPSFQG | | DY | |
| | | SEQ | | SEQ | | SEQ |
| | | ID | | ID | | ID |
| Antibody | LCDR1 | NO: | LCDR2 | NO: | LCDR3 | NO: |
| I3RB217 | RASQSVSS | 12 | GASSRAT | 13 | QQDYGFP | 14 |
| | SYL | | | | WT | |

TABLE 7
Heavy chain and light chain sequences of
anti-CD123 mAb.

| | | SEQ |
|---|---|---|
| | | ID |
| mAb ID | | NO: |
| | Heavy Chain Amino Acid Sequence | |
| I3RB217 | EVQLVQSGAEVKKPGESLKISCKGSGYSFTSYWI | 15 |
| | SWVRQMPGKGLEWMGIIDPSDSDTRYSPSFQGQV | |
| | TISADKSISTAYLQWSSLKASDTAMYYCARGDGS | |
| | TDLDYWGQGTLVTVSS | |
| | Light Chain Amino Acid Sequence | |
| I3RB217 | EIVLTQSPGTLSLSPGERATLSCRASQSVSSSYL | 16 |
| | AWYQQKPGQAPRLLIYGASSRATGIPDRFSGSGS | |
| | GTDFTLTISRLEPEDFAVYYCQQDYGFPWTFGQG | |
| | TKVEIK | |

TABLE 8
Sequences of half antibodies expressed in CHO cells.

| | | SEQ |
|---|---|---|
| | | ID |
| mAb ID | ‘Knob’ arm and ‘hole’ arm amino acid sequence | NO: |
| VG1 | MAWVWTLLFLMAAAQSIQADIVMSQSPSSLAVSVGEKVTMSCKSSQSLLYS | 17 |
| (ANTI- | SNQKNYLAWYQQKPGQSPKLLIYWASTRESGVPDRFTGSGSGTDFTLTISSV | |
| TRGV9 | KAEDLAVYYCQQYYRYHTFGTGTKLEIKRTVAAPSVFIFPPSDEQLKSGTAS | |
| half | VVCLLNNFYPREAKVQWKVDNALQSGNSQESVTEQDSKDSTYSLSSTLTLS | |
| antibody) | KADYEKHKVYACEVTHQGLSSPVTKSFNRGECGGSEGKSSGSGSESKSTEG | |
| | KSSGSGSESKSTGGSEVQLQQSGAELARPGASVKLSCKASGFTFTDHYINWV | |
| | KQRTGQGLEWIGQIYPGDGNTYYNQKFKGKATLTADKSSSTAYMQLSSLTS | |
| | EDSAVYFCAPNYGDYTIDFWGQGTSVTVSSASTKGPSVFPLAPCSRSTSEST | |
| | AALGCLVKDYFPEPVTVSWNSGALTSGVHTFPAVLQSSGLYSLSSVVTVPSS | |
| | SLGTKTYTCNVDHKPSNTKVDKRVESKYGPPCPPCPAPEAAGGPSVFLFPPK | |
| | PKDTLMISRTPEVTCVVVDVSQEDPEVQFNWYVDGVEVHNAKTKPREEQF | |
| | NSTYRVVSVLTVLHQDWLNGKEYKCKVSNKGLPSSIEKTISKAKGQPREPQ | |
| | VYTLPPSQEEMTKNQVSLSCAVKGFYPSDIAVEWESNGQPENNYKTTPPVL | |
| | DSDGSFFLVSRLTVDKSRWQEGNVFSCSVMHEALHNRFTQKSLSLSLGK | |
| VG1 | MAWVWTLLFLMAAAQSIQAEIVLTQSPGTLSLSPGERATLSCRASQSVSSSY | 18 |
| (anti- | LAWYQQKPGQAPRLLIYGASSRATGIPDRFSGSGSGTDFTLTISRLEPEDFAV | |
| CD123 | YYCQQDYGFPWTFGQGTKVEIKRTVAAPSVFIFPPSDEQLKSGTASVVCLLN | |
| half Ab) | NFYPREAKVQWKVDNALQSGNSQESVTEQDSKDSTYSLSSTLTLSKADYEK | |
| | HKVYACEVTHQGLSSPVTKSFNRGECGGSEGKSSGSGSESKSTEGKSSGSGS | |
| | ESKSTGGSEVQLVQSGAEVKKPGESLKISCKGSGYSFTSYWISWVRQMPGK | |
| | GLEWMGIIDPSDSDTRYSPSFQGQVTISADKSISTAYLQWSSLKASDTAMYY | |
| | CARGDGSTDLDYWGQGTLVTVSSASTKGPSVFPLAPCSRSTSESTAALGCLV | |
| | KDYFPEPVTVSWNSGALTSGVHTFPAVLQSSGLYSLSSVVTVPSSSLGTKTY | |
| | TCNVDHKPSNTKVDKRVESKYGPPCPPCPAPEAAGGPSVFLFPPKPKDTLMI | |
| | SRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVYTLPPSQ | |
| | EEMTKNQVSLWCLVKGFYPSDIAVEWESNGQPENNYKTTPPVLDSDGSFFL | |
| | YSRLTVDKSRWQEGNVFSCSVMHEALHNHYTQKSLSLSLGK | |
| VG3 | MAWVWTLLFLMAAAQSIQAEIVLTQSPGTLSLSPGERATLSCRASQSVSSSY | 19 |
| (B23B49 | LAWYQQKPGQAPRLLIYGASSRATGIPDRFSGSGSGTDFTLTISRLEPEDFAV | |
| Null half | YYCQQDYGFPWTFGQGTKVEIKRTVAAPSVFIFPPSDEQLKSGTASVVCLLN | |
| Ab) | NFYPREAKVQWKVDNALQSGNSQESVTEQDSKDSTYSLSSTLTLSKADYEK | |
| | HKVYACEVTHQGLSSPVTKSFNRGECGGSEGKSSGSGSESKSlEGKSSGSGS | |
| | ESKSTGGSEVQLVQSGAEVKKPGESLKISCKGSGYSFTSYWISWVRQMPGK | |
| | GLEWMGIIDPSDSDTRYSPSFQGQVTISADKSISTAYLQWSSLKASDTAMYY | |
| | CARGDGSTDLDYWGQGTLVTVSSASTKGPSVFPLAPCSRSTSESTAALGCLV | |
| | KDYFPEPVTVSWNSGALTSGVHTFPAVLQSSGLYSLSSVVTVPSSSLGTKTY | |
| | TCNVDHKPSNTKVDKRVESKYGPPCPPCPAPEAAGGPSVFLFPPKPKDTLMI | |
| | SRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVYTLPPSQ | |
| | EEMTKNQVSLWCLVKGFYPSDIAVEWESNGQPENNYKTTPPVLDSDGSFFL | |
| | YSRLTVDKSRWQEGNVFSCSVMHEALHNHYTQKSLSLSLGK | |
| | Half Antibody DNA sequence | |
| VG1 | ATGGCCTGGGTGTGGACCCTGCTGTTCCTGATGGCCGCCGCCCAGAGCAT | 20 |
| (ANTI- | CCAGGCCGACATCGTGATGAGCCAGAGCCCAAGCAGCCTGGCCGTGAGC | |
| TRGV9 | GTGGGCGAGAAGGTGACCATGAGCTGCAAGAGCAGCCAGAGCCTGCTGT | |
| half Ab) | ACAGCAGCAACCAGAAGAACTACCTGGCCTGGTACCAGCAGAAGCCAG | |
| | GCCAGAGCCCAAAGCTGCTGATCTACTGGGCCAGCACCCGCGAGAGCGG | |
| | CGTGCCAGACCGCTTCACCGGCAGCGGCAGCGGCACCGACTTCACCCTG | |
| | ACCATCAGCAGCGTGAAGGCCGAGGACCTGGCCGTGTACTACTGCCAGC | |
| | AGTACTACCGCTACCACACCTTCGGCACCGGCACCAAGCTGGAGATCAA | |
| | GCGCACCGTGGCCGCCCCAAGCGTGTTCATCTTCCCACCAAGCGACGAG | |
| | CAGCTGAAGAGCGGCACCGCCAGCGTGGTGTGCCTGCTGAACAACTTCT | |
| | ACCCACGCGAGGCCAAGGTGCAGTGGAAGGTGGACAACGCCCTGCAGA | |
| | GCGGCAACAGCCAGGAGAGCGTGACCGAGCAGGACAGCAAGGACAGCA | |
| | CCTACAGCCTGAGCAGCACCCTGACCCTGAGCAAGGCCGACTACGAGAA | |
| | GCACAAGGTGTACGCCTGCGAGGTGACCCACCAGGGCCTGAGCAGCCCA | |
| | GTGACCAAGAGCTTCAACCGCGGCGAGTGCggcggcagcgagggcaaga | |
| | gcagcggcagcggcagcgagagcaagagcaccg | |
| | agggcaagagcagcggcagcggcagcgagagcaagagcaccggcggca | |
| | gcGAGGTGCAGCTGCAGCAGAGCGGCGCCGAGCTGGCCCGCCCAGGCGC | |
| | CAGCGTGAAGCTGAGCTGCAAGGCCAGCGGCTTCACCTTCACCGACCAC | |
| | TACATCAACTGGGTGAAGCAGCGCACCGGCCAGGGCCTGGAGTGGATCG | |
| | GCCAGATCTACCCAGGCGACGGCAACACCTACTACAACCAGAAGTTCAA | |
| | GGGCAAGGCCACCCTGACCGCCGACAAGAGCAGCAGCACCGCCTACATG | |
| | CAGCTGAGCAGCCTGACCAGCGAGGACAGCGCCGTGTACTTCTGCGCCC | |
| | CAAACTACGGCGACTACACCATCGACTTCTGGGGCCAGGGCACCAGCGT | |
| | GACCGTGAGCAGCGCCAGCACCAAGGGCCCAAGCGTGTTCCCACTGGCC | |
| | CCATGCAGCCGCAGCACCAGCGAGAGCACCGCCGCCCTGGGCTGCCTGG | |
| | TGAAGGACTACTTCCCAGAGCCAGTGACCGTGAGCTGGAACAGCGGCGC | |
| | CCTGACCAGCGGCGTGCACACCTTCCCAGCCGTGCTGCAGAGCAGCGGC | |
| | CTGTACAGCCTGAGCAGCGTGGTGACCGTGCCAAGCAGCAGCCTGGGCA | |
| | CCAAGACCTACACCTGCAACGTGGACCACAAGCCAAGCAACACCAAGGT | |
| | GGACAAGCGCGTGGAGAGCAAGTACGGCCCACCATGCCCACCATGCCCA | |
| | GCCCCAGAGGCCGCCGGCGGCCCAAGCGTGTTCCTGTTCCCACCAAAGC | |
| | CAAAGGACACCCTGATGATCAGCCGCACCCCAGAGGTGACCTGCGTGGT | |
| | GGTGGACGTGAGCCAGGAGGACCCAGAGGTGCAGTTCAACTGGTACGTG | |
| | GACGGCGTGGAGGTGCACAACGCCAAGACCAAGCCACGCGAGGAGCAG | |
| | TTCAACAGCACCTACCGCGTGGTGAGCGTGCTGACCGTGCTGCACCAGG | |
| | ACTGGCTGAACGGCAAGGAGTACAAGTGCAAGGTGAGCAACAAGGGCC | |
| | TGCCAAGCAGCATCGAGAAGACCATCAGCAAGGCCAAGGGCCAGCCAC | |
| | GCGAGCCACAGGTGTACACCCTGCCACCAAGCCAGGAGGAGATGACCA | |
| | AGAACCAGGTGAGCCTGTGGTGCCTGGTGAAGGGCTTCTACCCAAGCGA | |
| | CATCGCCGTGGAGTGGGAGAGCAACGGCCAGCCAGAGAACAACTACAA | |
| | GACCACCCCACCAGTGCTGGACAGCGACGGCAGCTTCTTCCTGTACAGC | |
| | CGCCTGACCGTGGACAAGAGCCGCTGGCAGGAGGGCAACGTGTTCAGCT | |
| | GCAGCGTGATGCACGAGGCCCTGCACAACCACTACACCCAGAAGAGCCT | |
| | GAGCCTGAGCCTGGGCAAG | |
| VG1 | ATGGCCTGGGTGTGGACCCTGCTGTTCCTGATGGCCGCCGCCCAGAGCAT | 21 |
| (anti- | CCAGGCCGAGATCGTGCTGACCCAGAGCCCAGGCACCCTGAGCCTGAGC | |
| CD123 | CCAGGCGAGCGCGCCACCCTGAGCTGCCGCGCCAGCCAGAGCGTGAGCA | |
| half Ab) | GCAGCTACCTGGCCTGGTACCAGCAGAAGCCAGGCCAGGCCCCACGCCT | |
| | GCTGATCTACGGCGCCAGCAGCCGCGCCACCGGCATCCCAGACCGCTTC | |
| | AGCGGCAGCGGCAGCGGCACCGACTTCACCCTGACCATCAGCCGCCTGG | |
| | AGCCAGAGGACTTCGCCGTGTACTACTGCCAGCAGGACTACGGCTTCCC | |
| | ATGGACCTTCGGCCAGGGCACCAAGGTGGAGATCAAGCGCACCGTGGCC | |
| | GCCCCAAGCGTGTTCATCTTCCCACCAAGCGACGAGCAGCTGAAGAGCG | |
| | GCACCGCCAGCGTGGTGTGCCTGCTGAACAACTTCTACCCACGCGAGGC | |
| | CAAGGTGCAGTGGAAGGTGGACAACGCCCTGCAGAGCGGCAACAGCCA | |
| | GGAGAGCGTGACCGAGCAGGACAGCAAGGACAGCACCTACAGCCTGAG | |
| | CAGCACCCTGACCCTGAGCAAGGCCGACTACGAGAAGCACAAGGTGTAC | |
| | GCCTGCGAGGTGACCCACCAGGGCCTGAGCAGCCCAGTGACCAAGAGCT | |
| | TCAACCGCGGCGAGTGCggcggcagcgagggcaagagcagcggcagcgg | |
| | cagcgagagcaagagcaccgagggcaagagcag | |
| | cggcagcggcagcgagagcaagagcaccggcggcagcGAGGTGCAGCT | |
| | GGTGCAGAGCGGCGCCGAGGTGAAGAAGCCAGGCGAGAGCCTGAAGAT | |
| | CAGCTGCAAGGGCAGCGGCTACAGCTTCACCAGCTACTGGATCAGCTGG | |
| | GTGCGCCAGATGCCAGGCAAGGGCCTGGAGTGGATGGGCATCATCGACC | |
| | CAAGCGACAGCGACACCCGCTACAGCCCAAGCTTCCAGGGCCAGGTGAC | |
| | CATCAGCGCCGACAAGAGCATCAGCACCGCCTACCTGCAGTGGAGCAGC | |
| | CTGAAGGCCAGCGACACCGCCATGTACTACTGCGCCCGCGGCGACGGCA | |
| | GCACCGACCTGGACTACTGGGGCCAGGGCACCCTGGTGACCGTGAGCAG | |
| | CGCCAGCACCAAGGGCCCAAGCGTGTTCCCACTGGCCCCATGCAGCCGC | |
| | AGCACCAGCGAGAGCACCGCCGCCCTGGGCTGCCTGGTGAAGGACTACT | |
| | TCCCAGAGCCAGTGACCGTGAGCTGGAACAGCGGCGCCCTGACCAGCGG | |
| | CGTGCACACCTTCCCAGCCGTGCTGCAGAGCAGCGGCCTGTACAGCCTG | |
| | AGCAGCGTGGTGACCGTGCCAAGCAGCAGCCTGGGCACCAAGACCTACA | |
| | CCTGCAACGTGGACCACAAGCCAAGCAACACCAAGGTGGACAAGCGCG | |
| | TGGAGAGCAAGTACGGCCCACCATGCCCACCATGCCCAGCCCCAGAGGC | |
| | CGCCGGCGGCCCAAGCGTGTTCCTGTTCCCACCAAAGCCAAAGGACACC | |
| | CTGATGATCAGCCGCACCCCAGAGGTGACCTGCGTGGTGGTGGACGTGA | |
| | GCCAGGAGGACCCAGAGGTGCAGTTCAACTGGTACGTGGACGGCGTGGA | |
| | GGTGCACAACGCCAAGACCAAGCCACGCGAGGAGCAGTTCAACAGCAC | |
| | CTACCGCGTGGTGAGCGTGCTGACCGTGCTGCACCAGGACTGGCTGAAC | |
| | GGCAAGGAGTACAAGTGCAAGGTGAGCAACAAGGGCCTGCCAAGCAGC | |
| | ATCGAGAAGACCATCAGCAAGGCCAAGGGCCAGCCACGCGAGCCACAG | |
| | GTGTACACCCTGCCACCAAGCCAGGAGGAGATGACCAAGAACCAGGTG | |
| | AGCCTGTGGTGCCTGGTGAAGGGCTTCTACCCAAGCGACATCGCCGTGG | |
| | AGTGGGAGAGCAACGGCCAGCCAGAGAACAACTACAAGACCACCCCAC | |
| | CAGTGCTGGACAGCGACGGCAGCTTCTTCCTGTACAGCCGCCTGACCGT | |
| | GGACAAGAGCCGCTGGCAGGAGGGCAACGTGTTCAGCTGCAGCGTGATG | |
| | CACGAGGCCCTGCACAACCACTACACCCAGAAGAGCCTGAGCCTGAGCC | |
| | TGGGCAAG | |
| VG3 | ATGGCCTGGGTGTGGACCCTGCTGTTCCTGATGGCCGCCGCCCAGAGCAT | 22 |
| (Null half | CCAGGCCGACATCGTGATGACCCAGAGCCCAGACAGCCTGGCCGTGAGC | |
| Ab) | CTGGGCGAGCGCGCCACCATCAACTGCCGCGCCAGCCAGAGCGTGGACT | |
| | ACAACGGCATCAGCTACATGCACTGGTACCAGCAGAAGCCAGGCCAGCC | |
| | ACCAAAGCTGCTGATCTACGCCGCCAGCAACCCAGAGAGCGGCGTGCCA | |
| | GACCGCTTCAGCGGCAGCGGCAGCGGCACCGACTTCACCCTGACCATCA | |
| | GCAGCCTGCAGGCCGAGGACGTGGCCGTGTACTACTGCCAGCAGATCAT | |
| | CGAGGACCCATGGACCTTCGGCCAGGGCACCAAGGTGGAGATCAAGCGC | |
| | ACCGTGGCCGCCCCAAGCGTGTTCATCTTCCCACCAAGCGACGAGCAGC | |
| | TGAAGAGCGGCACCGCCAGCGTGGTGTGCCTGCTGAACAACTTCTACCC | |
| | ACGCGAGGCCAAGGTGCAGTGGAAGGTGGACAACGCCCTGCAGAGCGG | |
| | CAACAGCCAGGAGAGCGTGACCGAGCAGGACAGCAAGGACAGCACCTA | |
| | CAGCCTGAGCAGCACCCTGACCCTGAGCAAGGCCGACTACGAGAAGCAC | |
| | AAGGTGTACGCCTGCGAGGTGACCCACCAGGGCCTGAGCAGCCCAGTGA | |
| | CCAAGAGCTTCAACCGCGGCGAGTGCGGCGGCAGCGAGGGCAAGAGCA | |
| | GCGGCAGCGGCAGCGAGAGCAAGAGCACCGAGGGCAAGAGCAGCGGCA | |
| | GCGGCAGCGAGAGCAAGAGCACCGGCGGCAGCCAGATCACCCTGAAGG | |
| | AGAGCGGCCCAACCCTGGTGAAGCCAACCCAGACCCTGACCCTGACCTG | |
| | CACCTTCAGCGGCTTCAGCCTGAGCACCAGCGGCATGGGCGTGAGCTGG | |
| | ATCCGCCAGCCACCAGGCAAGGCCCTGGAGTGGCTGGCCCACATCTACT | |
| | GGGACGACGACAAGCGCTACAACCCAAGCCTGAAGAGCCGCCTGACCAT | |
| | CACCAAGGACACCAGCAAGAACCAGGTGGTGCTGACCATGACCAACATG | |
| | GACCCAGTGGACACCGCCACCTACTACTGCGCCCGCCTGTACGGCTTCAC | |
| | CTACGGCTTCGCCTACTGGGGCCAGGGCACCCTGGTGACCGTGAGCAGC | |
| | GCCAGCACCAAGGGCCCAAGCGTGTTCCCACTGGCCCCATGCAGCCGCA | |
| | GCACCAGCGAGAGCACCGCCGCCCTGGGCTGCCTGGTGAAGGACTACTT | |
| | CCCAGAGCCAGTGACCGTGAGCTGGAACAGCGGCGCCCTGACCAGCGGC | |
| | GTGCACACCTTCCCAGCCGTGCTGCAGAGCAGCGGCCTGTACAGCCTGA | |
| | GCAGCGTGGTGACCGTGCCAAGCAGCAGCCTGGGCACCAAGACCTACAC | |
| | CTGCAACGTGGACCACAAGCCAAGCAACACCAAGGTGGACAAGCGCGT | |
| | GGAGAGCAAGTACGGCCCACCATGCCCACCATGCCCAGCCCCAGAGGCC | |
| | GCCGGCGGCCCAAGCGTGTTCCTGTTCCCACCAAAGCCAAAGGACACCC | |
| | TGATGATCAGCCGCACCCCAGAGGTGACCTGCGTGGTGGTGGACGTGAG | |
| | CCAGGAGGACCCAGAGGTGCAGTTCAACTGGTACGTGGACGGCGTGGAG | |
| | GTGCACAACGCCAAGACCAAGCCACGCGAGGAGCAGTTCAACAGCACCT | |
| | ACCGCGTGGTGAGCGTGCTGACCGTGCTGCACCAGGACTGGCTGAACGG | |
| | CAAGGAGTACAAGTGCAAGGTGAGCAACAAGGGCCTGCCAAGCAGCAT | |
| | CGAGAAGACCATCAGCAAGGCCAAGGGCCAGCCACGCGAGCCACAGGT | |
| | GTACACCCTGCCACCAAGCCAGGAGGAGATGACCAAGAACCAGGTGAG | |
| | CCTGTGGTGCCTGGTGAAGGGCTTCTACCCAAGCGACATCGCCGTGGAG | |
| | TGGGAGAGCAACGGCCAGCCAGAGAACAACTACAAGACCACCCCACCA | |
| | GTGCTGGACAGCGACGGCAGCTTCTTCCTGTACAGCCGCCTGACCGTGG | |
| | ACAAGAGCCGCTGGCAGGAGGGCAACGTGTTCAGCTGCAGCGTGATGCA | |
| | CGAGGCCCTGCACAACCACTACACCCAGAAGAGCCTGAGCCTGAGCCTG | |
| | GGCAAG | |

TABLE 9
Heavy and Light Chain Sequences for TRGV9 bispecific antibodies

| Bispecific | | |
|---|---|---|
| Antibody | | Amino Acid Sequence |
| VG1 | Heavy chain 1 | EVQLQQSGAELARPGASVKLSCKASGFTFTDHYINWVKQRT |
| (ANTI- | VG1 (SEQ ID | GQGLEWIGQIYPGDGNTYYNQKFKGKATLTADKSSSTAYM |
| TRGV9/anti- | NO: 23) | QLSSLTSEDSAVYFCAPNYGDYTIDFWGQGTSVTVSSASTK |
| CD123) | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEAAGGPSVFLFPPKPKDT |
| | | LMISRTPEVTCVVVDVSQEDPEVQFNWYVDGVEVHNAKTK |
| | | PREEQFNSTYRVVSVLTVLHQDWLNGKEYKCKVSNKGLPS |
| | | SIEKTISKAKGQPREPQVYTLPPSQEEMTKNQVSLSCAVKGF |
| | | YPSDIAVEWESNGQPENNYKTTPPVLDSDGSFFLVSRLTVD |
| | | KSRWQEGNVFSCSVMHEALHNRFTQKSLSLSLGK |
| | Light Chain 1 | DIVMSQSPSSLAVSVGEKVTMSCKSSQSLLYSSNQKNYLAW |
| | VG1 (SEQ ID | YQQKPGQSPKLLIYWASTRESGVPDRFTGSGSGTDFTLTISS |
| | NO: 24) | VKAEDLAVYYCQQYYRYHTFGTGTKLEIKRTVAAPSVFIFP |
| | | PSDEQLKSGTASVVCLLNNFYPREAKVQWKVDNALQSGNS |
| | | QESVTEQDSKDSTYSLSSTLTLSKADYEKHKVYACEVTHQG |
| | | LSSPVTKSFNRGEC |
| | Heavy chain 2 | EVQLVQSGAEVKKPGESLKISCKGSGYSFTSYWISWVRQMP |
| | VG1 (SEQ ID | GKGLEWMGIIDPSDSDTRYSPSFQGQVTISADKSISTAYLQW |
| | NO: 25) | SSLKASDTAMYYCARGDGSTDLDYWGQGTLVTVSSASTKG |
| | | PSVFPLAPCSRSTSESTAALGCLVKDYFPEPVTVSWNSGALT |
| | | SGVHTFPAVLQSSGLYSLSSVVTVPSSSLGTKTYTCNVDHKP |
| | | SNTKVDKRVESKYGPPCPPCPAPEAAGGPSVFLFPPKPKDTL |
| | | MISRTPEVTCVVVDVSQEDPEVQFNWYVDGVEVHNAKTKP |
| | | REEQFNSTYRVVSVLTVLHQDWLNGKEYKCKVSNKGLPSSI |
| | | EKTISKAKGQPREPQVYTLPPSQEEMTKNQVSLWCLVKGFY |
| | | PSDIAVEWESNGQPENNYKTTPPVLDSDGSFFLYSRLTVDKS |
| | | RWQEGNVFSCSVMHEALHNHYTQKSLSLSLGK |
| | Light Chain 2 | EIVLTQSPGTLSLSPGERATLSCRASQSVSSSYLAWYQQKPG |
| | VG1 (SEQ ID | QAPRLLIYGASSRATGIPDRFSGSGSGTDFTLTISRLEPEDFA |
| | NO: 26) | VYYCQQDYGFPWTFGQGTKVEIKRTVAAPSVFIFPPSDEQL |
| | | KSGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC |
| ANTI- | Heavy chain 1 | QVQLQESGPGLVKPSETLSLTCTVSGYSITSGYFWNWIRQPP |
| TRGV9 x | VG3 (SEQ ID | GKGLEWIGYISYDGSNNYNPSLKSRVTISRDTSKNQFSLKLS |
| Null | NO: 27) | SVTAADTAVYYCASPSPGTGYAVDYWGQGTLVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEAAGGPSVFLFPPKPKDT |
| | | LMISRTPEVTCVVVDVSQEDPEVQFNWYVDGVEVHNAKTK |
| | | PREEQFNSTYRVVSVLTVLHQDWLNGKEYKCKVSNKGLPS |
| | | SIEKTISKAKGQPREPQVYTLPPSQEEMTKNQVSLSCAVKGF |
| | | YPSDIAVEWESNGQPENNYKTTPPVLDSDGSFFLVSRLTVD |
| | | KSRWQEGNVFSCSVMHEALHNRFTQKSLSLSLGK |
| | Light Chain 1 | DIQMTQSPSSLSASVGDRVTITCRSSQSLVHSNGNTYLHWY |
| | VG3 (SEQ ID | QQKPGKAPKFLIYKVSNRFSGVPSRFSGSGSGTDFTLTISSLQ |
| | NO: 28) | PEDFATYYCSQSTHVPFTFGQGTKLEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |
| | Heavy chain 2 | QITLKESGPTLVKPTQTLTLTCTFSGFSLSTSGMGVSWIRQPP |
| | VG3 (SEQ ID | GKALEWLAHIYWDDDKRYNPSLKSRLTITKDTSKNQVVLT |
| | NO: 29) | MTNMDPVDTATYYCARLYGFTYGFAYWGQGTLVTVSSAS |
| | | TKGPSVFPLAPCSRSTSESTAALGCLVKDYFPEPVTVSWNSG |
| | | ALTSGVHTFPAVLQSSGLYSLSSVVTVPSSSLGTKTYTCNVD |
| | | HKPSNTKVDKRVESKYGPPCPPCPAPEAAGGPSVFLFPPKPK |
| | | DTLMISRTPEVTCVVVDVSQEDPEVQFNWYVDGVEVHNAK |
| | | TKPREEQFNSTYRVVSVLTVLHQDWLNGKEYKCKVSNKGL |
| | | PSSIEKTISKAKGQPREPQVYTLPPSQEEMTKNQVSLWCLVK |
| | | GFYPSDIAVEWESNGQPENNYKTTPPVLDSDGSFFLYSRLTV |
| | | DKSRWQEGNVFSCSVMHEALHNHYTQKSLSLSLGK |
| | Light Chain 2 | DIVMTQSPDSLAVSLGERATINCRASQSVDYNGISYMHWYQ |
| | VG3 (SEQ ID | QKPGQPPKLLIYAASNPESGVPDRFSGSGSGTDFTLTISSLQA |
| | NO: 30) | EDVAVYYCQQIIEDPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |

### Example 2.3: Characterization of Vγ9+(γδ) T Cells and Pan T Cells

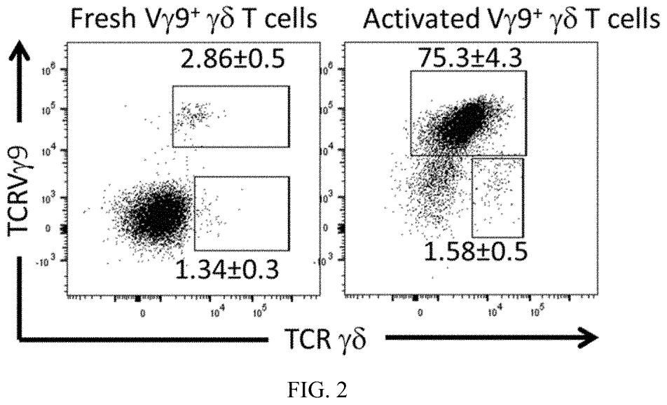

Zoledronic acid selectively expands Vγ9⁺γδ T cells from whole PBMCs. PBMCs were isolated from whole fresh PBMCs using EasySep™ Human γδ T cell isolation kit (Stem cell Technologies; Vancouver, Calif.) according to manufacturer instructions. Isolated PBMCs were cultured in RPMI-10 (RPMI supplemented with 10% FBS, 1× Pen/Strep) medium with recombinant human IL-2 (rhIL-2) to a final concentration of 1000 IU/mL and recombinant human IL-15 (rhIL-15) to a final concentration of 10 ng/mL and Zoledronic acid to a final concentration of 5 μM. for 14 days. Numbers in representative dot plots show the frequency (mean±SEM) of Vγ9⁺ and Vγ9⁻ TCR γδ T cells among total PBMCs on day 0 (left) and day 14 of PBMCs cultured with Zoledronic acid+IL-2+IL-15 (right). Represented data is mean (±SEM) of five donors (n=5) from a single experiment (FIG. 2).

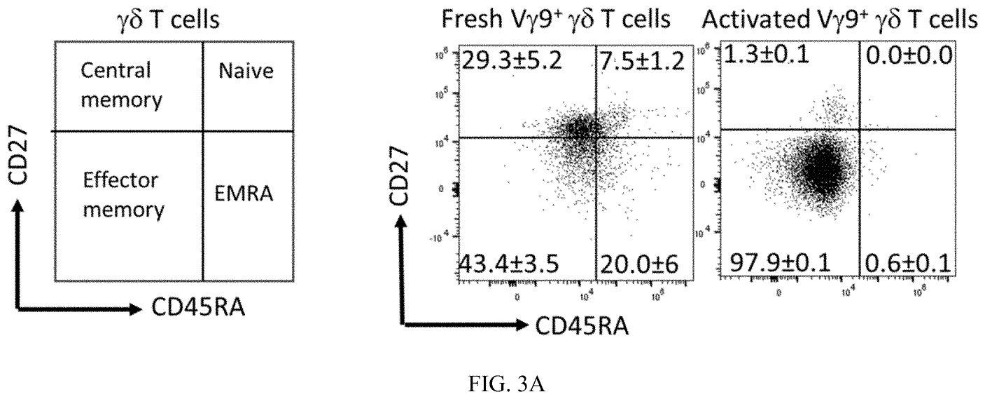

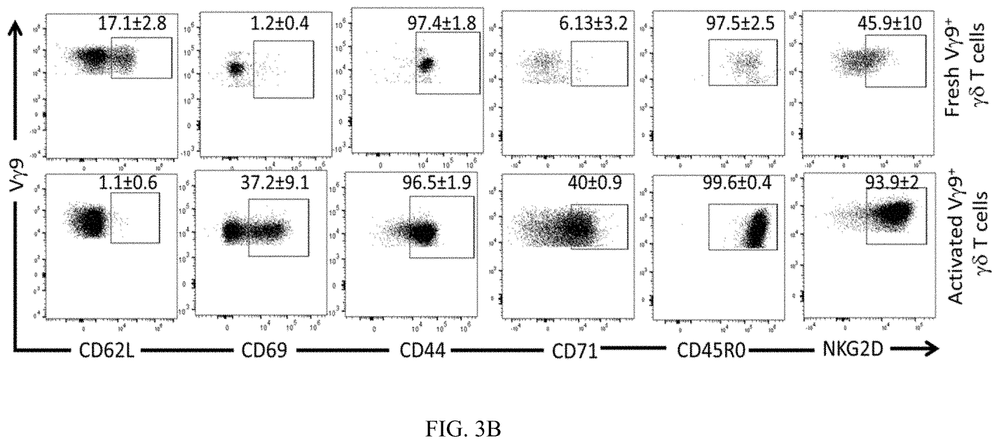

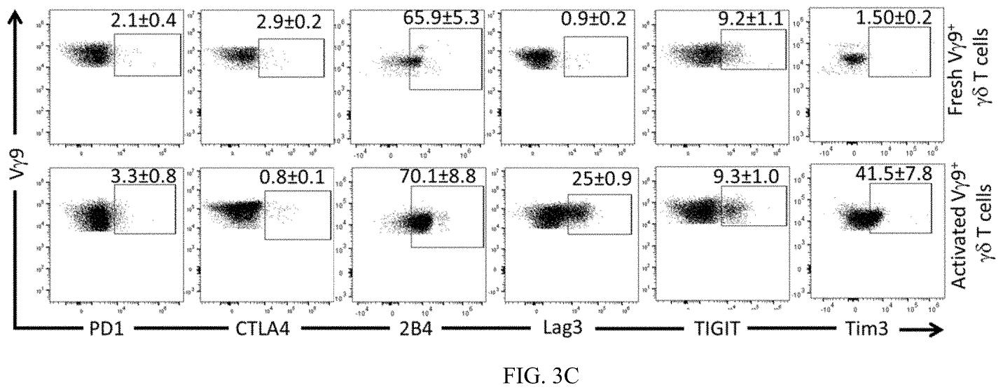

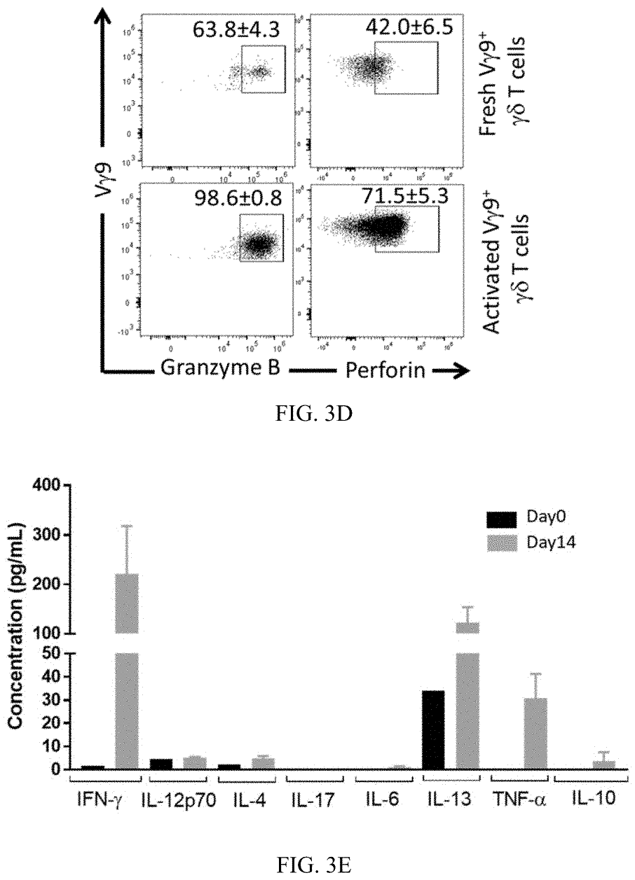

FIGS. 3A to 3E demonstrate the phenotypic characterization of Vγ9⁺γδ T cells. FIG. 3A shows a schematic depiction of gates used to describe the differentiation of γδ T cells (left). Representative FACS-dot plots show the differentiation profile of Vγ9⁺γδ T cells from fresh PBMCs (left) and PBMCs cultured ex vivo with Zoledronic acid+IL-2+IL-15 for 14 days (right). Numbers in quadrants mirror the frequency (mean±SEM) of the respective population among fresh and activated Vγ9⁺γδ T cells. Represented data is mean (±SEM) of five donors (n=5) from a single experiment. FIG. 3B shows numbers in representative dot plots mirroring the frequency (mean±SEM) of Vγ9⁺γδ T cells positive for respective activation marker either from fresh PBMCs (upper row) or PBMCs cultured with Zoledronic acid+IL-2+IL-15 for 14 days (lower row). Represented data is mean (±SEM) of seven donors (n=7) for CD62L, CD69, CD44 expression data from two independent experiments. n=5 donors for NKG2D and 2 donors for CD45RO and CD71 expression data respectively from a single experiment. FIG. 3C shows numbers above gates in dot plots depicting the frequency (mean±SEM) of Vγ9⁺γδ T cells positive for respective inhibitory receptor surface expression either from fresh PBMCs (upper row) or PBMCs cultured with Zoledronic acid+IL-2+IL-15 for day 14 days (lower row). Data shown here is mean (±SEM) of five donors (n=5) for PD1, CTLA4, TIGIT and Lag3 surface expression and seven donors (n=7) for 2B4 and Tim3 surface expression data from two independent experiments. FIG. 3D shows representative FACS dot plots demonstrating the frequency (mean±SEM) of Vγ9⁺γδ T cells expressing intracellular Granzyme B (left column) and Perforin (right column) from fresh PBMCs (upper row) and PBMCs cultured ex vivo with Zoledronic acid+IL-2+IL-15 for 14 days (lower row). Depicted data is mean (±SEM) of four (n=4) and seven (n=7) donors for Granzyme B and Perforin data respectively from two independent experiments. FIG. 3E shows bars representing the mean (±SEM) concentration (pg/mL) of cytokine in the cell culture supernatant on day 0 and day 14 of PBMCs culture with Zoledronic acid+IL-2+IL-15. Represented data is mean (±SEM) of four wells (n=4) from a single donor.

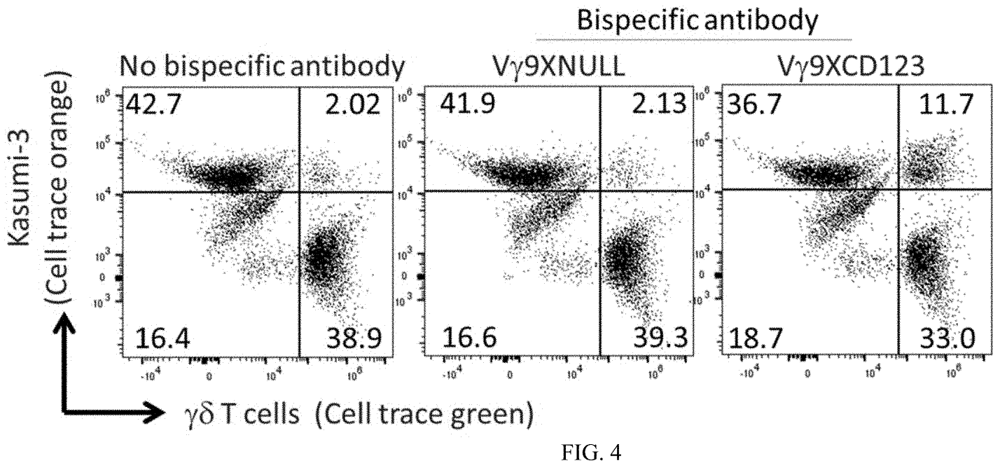

FIG. 4 shows that the anti-TRGV9/anti-CD123 bispecific antibody recruits) γδ T cells into biphasic cell-cell conjugate. γδ T cells (effector cells) were isolated from whole fresh PBMCs using EasySep™ Human γδ T cell isolation kit (Stem cell Technologies) according to manufacturer instructions. γδ T cells were labelled with 0.25 μM CellTracker™ Green CMFDA Dye for 30 Mins and Kasumi-3 (Targets) cells were labelled with 1 μM CellTracker™ Orange CMRA Dye in RPMI medium for 30 minutes at 37° C. Both labeled γδ T cells and Kasumi-3 were co-cultured. Labeled Effector (E) and Target (T) cells at an E:T ratio of 1:1 (50,000 cells of each cell type/well) with 1 microgram per ML of bispecific antibody (anti-TRGV9/anti-CD123, anti-TRGV9/anti-NULL) and incubated at 37° C. for 1 hour. At the end of the incubation, cells were spun down at 1200 rpm for 5 minutes and resuspended in FACS buffer. Cells were acquired utilizing the flow cytometer and analysis was performed using FLOWJO analysis software. Numbers in quadrants of representative FACS plots show the frequency of recruited or non-recruited cells to the cell-cell conjugate either in the absence (left dot plot) or presence of anti-TRGV9/anti-NULL (middle dot plot) and anti-TRGV9/anti-CD123 (right dot plot) bispecific antibody. Data shown here is from a single experiment.

### Example 2.4: Evaluation of Binding and Cytotoxic Properties of the Anti-Trgv9/Anti-Cd123 Bispecific Antibody Using Kasumi-3 Cells and Human γδ T Cells

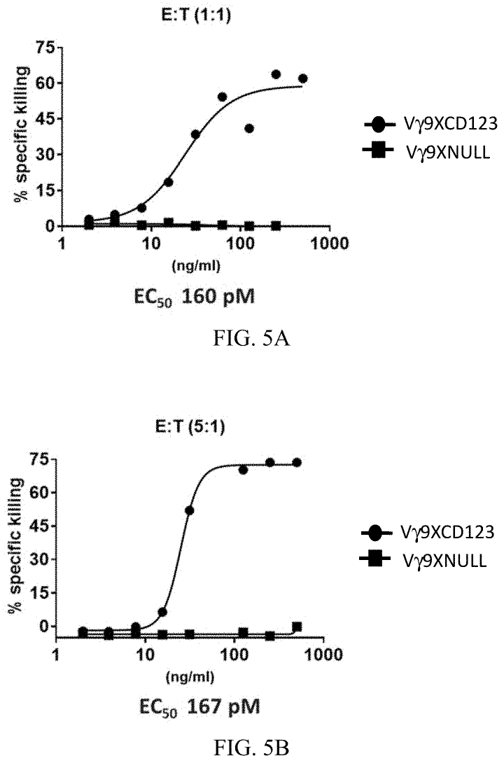

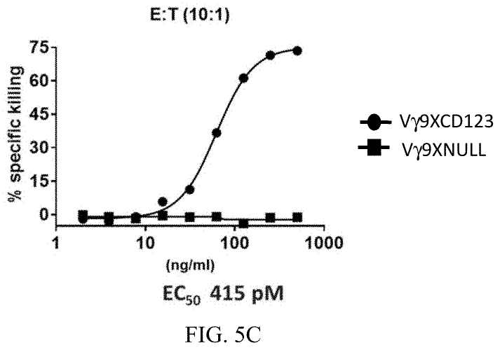

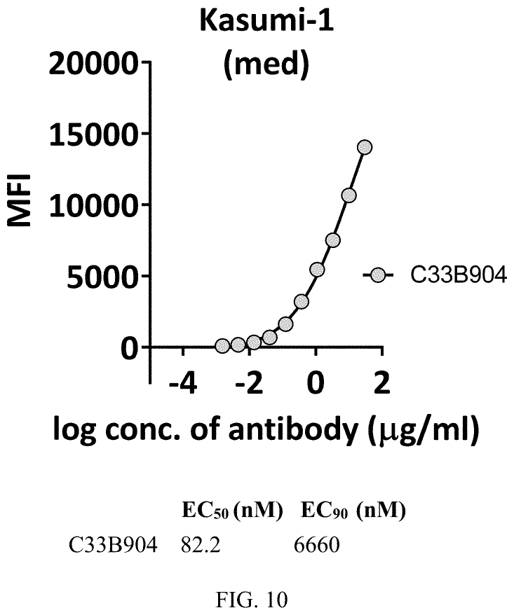

FIG. 5 shows that the anti-TRGV9/anti-CD123 bispecific antibody mediates γδ T cell cytotoxicity against CD123 expressing Kasumi-3 cells in vitro. Enriched γδ T cells (Effectors), isolated from PBMCs cultured with Zoledronic acid+IL-2+IL-15 for 12 days, were co-cultured with CFSE labelled Kasumi-3 cells (Targets) at 1:1, 5:1 and 10:1 E:T ratios in the presence of various concentrations of the bispecific antibody for 24 hours. Dose response curves show anti-TRGV9/anti-CD123 and anti-TRGV9/anti-NULL bispecific mediated γδ T cell cytotoxicity against CD123 expressing Kasumi-3 cells in a dose dependent manner at 1:1 (FIG. 5A) 5:1 (FIG. 5B) and 10:1 (FIG. 5C) E:T ratios. Cytotoxicity values represented here were subtracted of basal cytotoxicity value observed in the absence of bispecific antibody. EC₅₀ values were calculated as described in methods. Representative data shown here are from a single experiment.

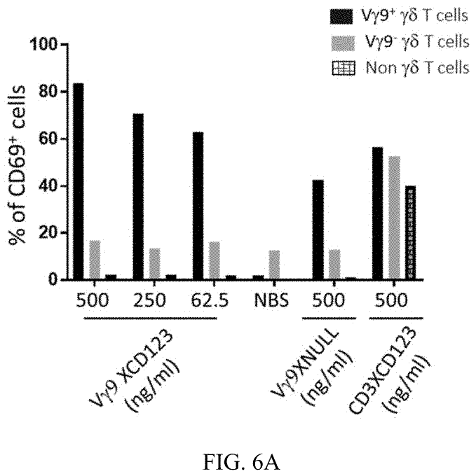

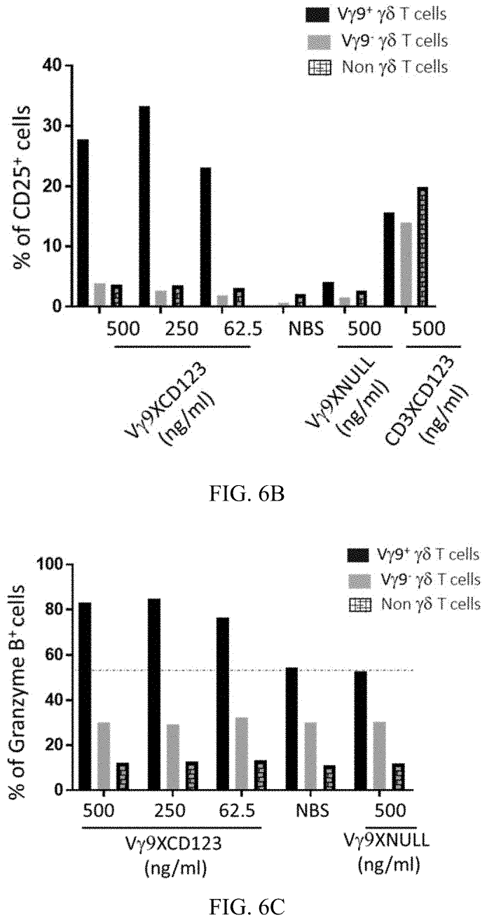

FIG. 6 shows that the anti-TRGV9/anti-CD123 bispecific selectively activates Vγ9⁺γδ T cells. Whole fresh PBMCs were co-cultured with Kasumi-3 cells in the presence of various concentrations of the anti-TRGV9/anti-CD123 bispecific antibody for 72 hours at 37° C. As a positive and negative control, co-cultured cells were stimulated with anti-CD3/anti-CD123 and anti-TRGV9/anti-NULL bispecifics for 72 hours at 37° C. Bars represent the frequency of Vγ9⁺, Vγ9⁻ γδ T cells and non-γδ T cells positive for CD69 (FIG. 6A, left), CD25 (FIG. 6A, right) surface expression, and intracellular Granzyme B (FIG. 6B) expression. The dotted line in FIG. 6B indicates the basal levels of Granzyme B expression in Vγ9⁺ γδ T cells. NBS denotes no bispecific antibody added to the co-cultured cells. Data shown here are from a single experiment.

### Example 3—Multispecific Antibodies that Bind Trgv9 and A Cancer Antigen

### Example 3.1—Preparation of Bispecific Antibodies that Bind TRGV9 and a Cancer Antigen

Variable region sequences of a TRGV9 monoclonal antibody and a second monoclonal antibody capable of binding an antigen on a T cell of interest are used to generate a bispecific antibody to be tested for γδ T cell re-directed killing of the target T cells.

Exemplary TRGV9 VH CDR1, VH CDR2, VH CDR3, VL CDR1, VL CDR2 and VL CDR3 sequences are provided in Table 1.1; and exemplary VH domain and VL domain sequences are provided in Table 1.2. However, any TRGV9 antibodies may be used for the preparation of the bispecific antibodies.

The second monoclonal antibody is an antibody that binds a cancer antigen as provided herein, including, without limitation a cancer antigen on the surface of a cancer cell.

Anti-TRGV9 bispecific antibodies are produced as full-length antibodies in the knob-into-hole format as human IgG4, as previously described (Atwell et al., J. Mol. Biol. 270:26-35 (1997)).

Nucleic acid sequences encoding variable regions are sub-cloned into custom mammalian expression vectors containing the constant region of IgG4 expression cassettes using standard PCR restriction enzyme based cloning techniques.

The bispecific antibodies are expressed by transient transfection in a CHO cell line. The antibodies are initially purified by MAB SELECT SURE Protein A column (GE Healthcare, Piscataway, N.J.) (Brown, Bottomley et al. Biochem Soc Trans. 1998 August; 26(3):5249). The column is equilibrated with PBS, pH 7.2 and is loaded with fermentation supernatant at a flow rate of 2 mL/min. After loading, the column is washed with PBS (4 column volumes (CV)) followed by elution in 30 mM sodium acetate, pH 3.5. Fractions containing protein peaks as monitored by absorbance at 280 nm in Akta Explorer (GE healthcare) are pooled together and are neutralized to pH 5.0 by adding 1% of 3M sodium acetate, pH 9.0. As a polishing step, the antibodies are purified on a preparative size exclusion chromatography (SEC) using a SUPERDEX 200 column (GE healthcare). The integrity of sample is assessed by endotoxin measurement and SDS polyacrylamide gel electrophoresis under reducing and non-reducing conditions. The final protein concentrations are determined.

### Example 3.2—Evaluation and Binding of Bispecific Antibodies that Bind TRGV9 and a T Cell Antigen

Assessment of binding of the bispecific antibody to Vγ9+ γδ T cells and target cells expressing the cancer antigen, and resulting cytotoxicity, will be determined in vitro.

Enriched γδ T cells (effectors), isolated from PBMCs cultured with zoledronic acid, IL-2, and IL-15 for 12 days, are co-cultured with CFSE-labelled cells expressing the T cell antigen (targets) at 1:1, 5:1 and 10:1 E:T ratios in the presence of various concentrations of the bispecific antibody for 24 hours. An anti-TRGV9/anti-NULL bispecific antibody will be used as a control. Cytotoxicity values are determined by subtracting basal cytotoxicity values observed in the absence of bispecific antibodies. Dose response curves are calculated to determine if bispecific mediated γδ T cell cytotoxicity occurs against the target cells expressing the cancer antigen in a dose dependent manner at 1:1, 5:1, and 10:1 E:T ratios.

In addition, selective activation of Vγ9+ γδ T cells is assessed by co-culturing whole fresh PBMCs with target cells expressing the cancer antigen in the presence of various concentrations of the anti-TRGV9/anti-cancer antigen bispecific antibody for 72 hours at 37° C. As a positive and negative control, co-cultured cells were stimulated with anti-CD3/anti-cancer antigen and anti-TRGV9/anti-NULL bispecifics for 72 hours at 37° C. The frequency of Vγ9⁺, Vγ9⁻γδ T cells and non-γδ T cells positive for CD69, CD25 surface expression, and intracellular Granzyme B expression is determined.

### Example 4—Multispecific Antibodies that Bind TRGV9 and CD33

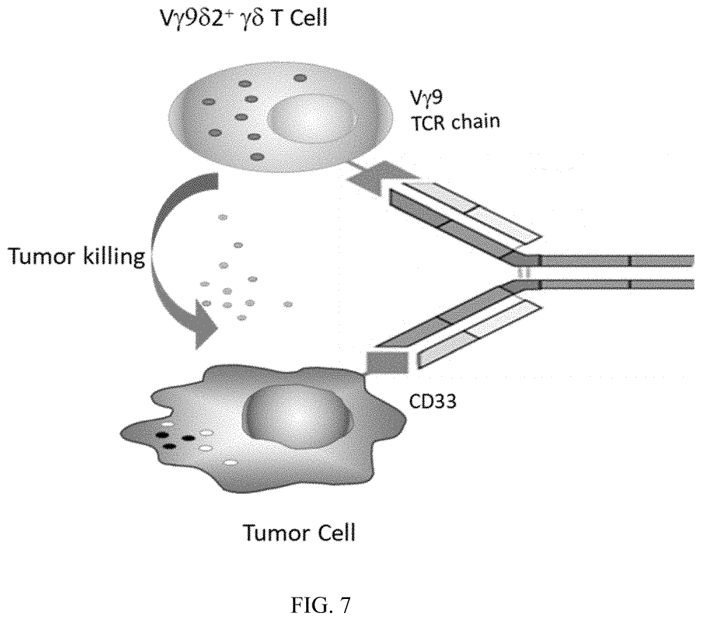

Examples 4.1-4.4 are based on the premise that γδ T cells, which mainly express heterodimers of TRGV9 and Vδ2 chains demonstrate potent anti-tumor functions. These cells express TCR-TRGV9 and the majority, if not all, of these cells exhibit efficient cytotoxicity of tumor target cells. This ability is then harnessed using bispecific antibodies constructed such that one arm binds to the TRGV9 structure and the other arm binds to a tumor-associated antigen (CD33) expressed by the tumor cells. Thus, the bispecific antibody bridges the effector and target cells together-resulting in tumor cell killing. This mechanism of action is described in the schematic outlined in FIG. 7.

The subsequent examples can be divided into the following categories: (1) Generation and characterization of bispecific antibodies capable of binding to the TRGV9 arm expressed on γδT cells and CD33 on cancer cells (Examples 4.1, 4.2, and 4.3); and (2) Evidence for bispecific antibody-enabled target cell killing by γδ T cells expanded in vitro (Example 4.4).

γδ T cell stimulation and expansion was performed. Expansion of Vγ9-Vδ2 T cells was carried out by treating PBMCs in complete RPMI media containing rhIL-2 (1000 IU/mL), rhIL-15 (10 ng/mL) and Zoledronic acid (5 μM) for 14 days.

### Example 4.1: Production of Anti-Trgv9 Mab

The mouse IgG1 anti-human T cell receptor anti-TRGV9 clone 7A5 was sourced commercially. Sample preparation and LC-MS/MS analysis were performed by Lake Pharma (San Carlos, Calif.). The sample was reduced and alkylated, divided into seven aliquots, and proteolytically digested with Trypsin/LysC, Chymotrypsin, LysC, Pepsin, and AspN, Elastase, and Proteinase K enzymes. Resulting peptides were desalted using a ZipTip C18 Pipette Tips and separated on-line using reverse phase chromatography. Mass spectrometry was performed on Thermo Q-Exactive spectrometer using HCD fragmentation. MS data sets were analyzed using PEAKS software by matching de novo sequence tags to an IMGT-based antibody sequences database. Gaps in the sequence were assigned using Contig sequence assembly of de novo identified peptides. All CDRs and hyper-mutations were confirmed by inspecting the MS/MS spectra.

The sequences of four monoclonal antibodies generated according to the above, as well as their CDR sequences, are shown previously in Tables 1-5.

### Example 4.2: Preparation of Anti-Trgv9/Anti-Cd33 Bispecific Antibodies

The variable region sequence of 7A5 (anti-TRGV9) and C33B904 (anti-CD33 antibody) (HCDRs and LCDRs in Table 10, HC and LC in Table 11) were used to generate a bispecific antibody to be tested for T cell re-directed killing of acute myeloid leukemia (AML) cells. The bispecific antibodies VG4 (anti-TRGV9×CD33) and VG3 (anti-TRGV9×Null) were produced as full-length antibodies in the knob-into-hole format as human IgG4. Nucleic acid sequences encoding variable regions were sub-cloned into a custom mammalian expression vectors containing constant region of human IgG4 expression cassettes using standard PCR restriction enzyme based standard cloning techniques, and sequenced verified. The bispecific antibodies were expressed by transient transfection in a Chinese hamster ovary (CHO) cell line. The sequences of the bispecific antibodies expressed in the CHO cells are shown in Table 12 below.

TABLE 10
CDR Sequences of anti-CD33 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| C33B904 | DYAMH | 37 | GIGWSGGSIVYADSVKG | 38 | DSPYGDFFDY | 39 |
| | | SEQ | | SEQ | | SEQ |
| | | ID | | ID | | ID |
| Antibody | LCDR1 | NO: | LCDR2 | NO: | LCDR3 | NO: |
| C33B904 | KSSQTVFYSSNNKNYLA | 40 | WASTRKS | 41 | QHYYSTPYT | 42 |

TABLE 11
Heavy chain and light chain sequences of
anti-CD33 mAb.

| | | SEQ |
|---|---|---|
| | | ID |
| mAb ID | Heavy Chain Amino Acid Sequence | NO: |
| C33B904 | EVQLVESGGGLVQPGRSLRLSCAASGFTFDDY | 43 |
| | AMHWVRQAPGKGLEWVSGIGWSGGSIVYADSV | |
| | KGRFTISRDNAKNSLYLQMNSLRAEDTALYYC | |
| | AKDSPYGDFFDYWGQGTLVTVSS | |
| | | SEQ |
| | | ID |
| | Light Chain Amino Acid Sequence | NO: |
| C33B904 | DIVMTQSPDSLAVSLGERATINCKSSQTVFYS | 44 |
| | SNNKNYLAWYQQKPGQPPKLLISWASTRKSGV | |
| | PDRFSGSGSGTDFTLTVSSLQAEDVAVYYCQH | |
| | YYSTPYTFGQGTKLEIK | |

TABLE 12
Sequences of antibodies expressed in CHO cells

| | | SEQ |
|---|---|---|
| | | ID |
| mAb ID | ‘Knob’ arm and ‘hole’ arm amino acid sequence | NO: |
| ANTI- | MAWVWTLLFLMAAAQSIQADIVMSQSPSSLAVSVGEKVTMSCKSSQSLLYS | 17 |
| TRGV9 | SNQKNYLAWYQQKPGQSPKLLIYWASTRESGVPDRFTGSGSGTDFTLTISSV | |
| (‘hole’ | KAEDLAVYYCQQYYRYHTFGTGTKLEIKRTVAAPSVFIFPPSDEQLKSGTAS | |
| arm) | VVCLLNNFYPREAKVQWKVDNALQSGNSQESVTEQDSKDSTYSLSSTLTLS | |
| | KADYEKHKVYACEVTHQGLSSPVTKSFNRGECGGSEGKSSGSGSESKSTEG | |
| | KSSGSGSESKSTGGSEVQLQQSGAELARPGASVKLSCKASGFTFTDHYINWV | |
| | KQRTGQGLEWIGQIYPGDGNTYYNQKFKGKATLTADKSSSTAYMQLSSLTS | |
| | EDSAVYFCAPNYGDYTIDFWGQGTSVTVSSASTKGPSVFPLAPCSRSTSEST | |
| | AALGCLVKDYFPEPVTVSWNSGALTSGVHTFPAVLQSSGLYSLSSVVTVPSS | |
| | SLGTKTYTCNVDHKPSNTKVDKRVESKYGPPCPPCPAPEAAGGPSVFLFPPK | |
| | PKDTLMISRTPEVTCVVVDVSQEDPEVQFNWYVDGVEVHNAKTKPREEQF | |
| | NSTYRVVSVLTVLHQDWLNGKEYKCKVSNKGLPSSIEKTISKAKGQPREPQ | |
| | VYTLPPSQEEMTKNQVSLSCAVKGFYPSDIAVEWESNGQPENNYKTTPPVL | |
| | DSDGSFFLVSRLTVDKSRWQEGNVFSCSVMHEALHNRFTQKSLSLSLGK | |
| anti- | MAWVWTLLFLMAAAQSIQADIVMTQSPDSLAVSLGERATINCKSSQTVFYS | 45 |
| CD33 | SNNKNYLAWYQQKPGQPPKLLISWASTRKSGVPDRFSGSGSGTDFTLTVSSL | |
| (‘knob’ | QAEDVAVYYCQHYYSTPYTFGQGTKLEIKRTVAAPSVFIFPPSDEQLKSGTA | |
| arm) | SVVCLLNNFYPREAKVQWKVDNALQSGNSQESVTEQDSKDSTYSLSSTLTL | |
| | SKADYEKHKVYACEVTHQGLSSPVTKSFNRGECGGSEGKSSGSGSESKSTE | |
| | GKSSGSGSESKSTGGSEVQLVESGGGLVQPGRSLRLSCAASGFTFDDYAMH | |
| | WVRQAPGKGLEWVSGIGWSGGSIVYADSVKGRFTISRDNAKNSLYLQMNS | |
| | LRAEDTALYYCAKDSPYGDFFDYWGQGTLVTVSSASTKGPSVFPLAPCSRS | |
| | TSESTAALGCLVKDYFPEPVTVSWNSGALTSGVHTFPAVLQSSGLYSLSSVV | |
| | TVPSSSLGTKTYTCNVDHKPSNTKVDKRVESKYGPPCPPCPAPEAAGGPSVF | |
| | LFPPKPKDTLMISRTPEVTCVVVDVSQEDPEVQFNWYVDGVEVHNAKTKPR | |
| | EEQFNSTYRVVSVLTVLHQDWLNGKEYKCKVSNKGLPSSIEKTISKAKGQP | |
| | REPQVYTLPPSQEEMTKNQVSLWCLVKGFYPSDIAVEWESNGQPENNYKTT | |
| | PPVLDSDGSFFLYSRLTVDKSRWQEGNVFSCSVMHEALHNHYTQKSLSLSL | |
| | GK | |
| Anti- | MAWVWTLLFLMAAAQSIQAEIVLTQSPGTLSLSPGERATLSCRASQSVSSSY | 46 |
| RSV | LAWYQQKPGQAPRLLIYGASSRATGIPDRFSGSGSGTDFTLTISRLEPEDFAV | |
| (‘knob’ | YYCQQDYGFPWTFGQGTKVEIKRTVAAPSVFIFPPSDEQLKSGTASVVCLLN | |
| arm) | NFYPREAKVQWKVDNALQSGNSQESVTEQDSKDSTYSLSSTLTLSKADYEK | |
| | HKVYACEVTHQGLSSPVTKSFNRGECGGSEGKSSGSGSESKSTEGKSSGSGS | |
| | ESKSTGGSEVQLVQSGAEVKKPGESLKISCKGSGYSFTSYWISWVRQMPGK | |
| | GLEWMGIIDPSDSDTRYSPSFQGQVTISADKSISTAYLQWSSLKASDTAMYY | |
| | CARGDGSTDLDYWGQGTLVTVSSASTKGPSVFPLAPCSRSTSESTAALGCLV | |
| | KDYFPEPVTVSWNSGALTSGVHTFPAVLQSSGLYSLSSVVTVPSSSLGTKTY | |
| | TCNVDHKPSNTKVDKRVESKYGPPCPPCPAPEAAGGPSVFLFPPKPKDTLMI | |
| | SRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVYTLPPSQ | |
| | EEMTKNQVSLWCLVKGFYPSDIAVEWESNGQPENNYKTTPPVLDSDGSFFL | |
| | YSRLTVDKSRWQEGNVFSCSVMHEALHNHYTQKSLSLSLGK | |

TABLE 13
Anti-TRGV9 and Anti-CD33 Heavy and Light Chain Sequences

| Antibody | Heavy Chain | Light Chain |
|---|---|---|
| TRGV9Ab_1 | EVQLQQSGAELARPGASVKLSCKASG | DIVMSQSPSSLAVSVGEKVTMSCKSSQSLLY |
| | FTFTDHYINWVKQRTGQGLEWIGQIY | SSNQKNYLAWYQQKPGQSPKLLIYWASTRES |
| | PGDGNTYYNQKFKGKATLTADKSSST | GVPDRFTGSGSGTDFTLTISSVKAEDLAVYY |
| | AYMQLSSLTSEDSAVYFCAPNYGDYT | CQQYYRYHTFGTGTKLEIKRTVAAPSVFIFP |
| | IDFWGQGTSVTVSSASTKGPSVFPLA | PSDEQLKSGTASVVCLLNNFYPREAKVQWKV |
| | PCSRSTSESTAALGCLVKDYFPEPVT | DNALQSGNSQESVTEQDSKDSTYSLSSTLTL |
| | VSWNSGALTSGVHTFPAVLQSSGLYS | SKADYEKHKVYACEVTHQGLSSPVTKSFNRG |
| | LSSVVTVPSSSLGTKTYTCNVDHKPS | EC (SEQ ID NO: 24) |
| | NTKVDKRVESKYGPPCPPCPAPEAAG | |
| | GPSVFLFPPKPKDTLMISRTPEVTCV | |
| | VVDVSQEDPEVQFNWYVDGVEVHNAK | |
| | TKPREEQFNSTYRVVSVLTVLHQDWL | |
| | NGKEYKCKVSNKGLPSSIEKTISKAK | |
| | GQPREPQVYTLPPSQEEMTKNQVSLS | |
| | CAVKGFYPSDIAVEWESNGQPENNYK | |
| | TTPPVLDSDGSFFLVSRLTVDKSRWQ | |
| | EGNVFSCSVMHEALHNRFTQKSLSLS | |
| | LGK (SEQ ID NO: 23) | |
| C33B904 | EVQLVESGGGLVQPGRSLRLSCAASG | DIVMTQSPDSLAVSLGERATINCKSSQTVFY |
| | FTFDDYAMHWVRQAPGKGLEWVSGIG | SSNNKNYLAWYQQKPGQPPKLLISWASTRKS |
| | WSGGSIVYADSVKGRFTISRDNAKNS | GVPDRFSGSGSGTDFTLTVSSLQAEDVAVYY |
| | LYLQMNSLRAEDTALYYCAKDSPYGD | CQHYYSTPYTFGQGTKLEIKRTVAAPSVFIF |
| | FFDYWGQGTLVTVSSASTKGPSVFPL | PPSDEQLKSGTASVVCLLNNFYPREAKVQWK |
| | APCSRSTSESTAALGCLVKDYFPEPV | VDNALQSGNSQESVTEQDSKDSTYSLSSTLT |
| | TVSWNSGALTSGVHTFPAVLQSSGLY | LSKADYEKHKVYACEVTHQGLSSPVTKSFNR |
| | SLSSVVTVPSSSLGTKTYTCNVDHKP | GEC (SEQ ID NO: 48) |
| | SNTKVDKRVESKYGPPCPPCPAPEAA | |
| | GGPSVFLFPPKPKDTLMISRTPEVTC | |
| | VVVDVSQEDPEVQFNWYVDGVEVHNA | |
| | KTKPREEQFNSTYRVVSVLTVLHQDW | |
| | LNGKEYKCKVSNKGLPSSIEKTISKA | |
| | KGQPREPQVYTLPPSQEEMTKNQVSL | |
| | WCLVKGFYPSDIAVEWESNGQPENNY | |
| | KTTPPVLDSDGSFFLYSRLTVDKSRW | |
| | QEGNVFSCSVMHEALHNHYTQKSLSL | |
| | SLGK (SEQ ID NO: 47) | |

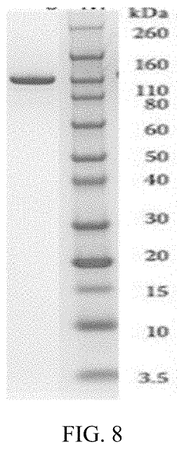

The antibodies were initially purified by Mab Select SuRe Protein A column (GE Healthcare). The column was equilibrated with PBS pH 7.2 and loaded with fermentation supernatant at a flow rate of 2 mL/min. After loading, the column was washed with 4 column volumes of PBS followed by elution in 30 mM sodium acetate, pH 3.5. Fractions containing protein peaks as monitored by absorbance at 280 nm were pooled and neutralized to pH 5.0 by adding 1% 3 M sodium acetate pH 9.0. The bispecific mAbs were further purified on a preparative Superdex 200 10/300 GL (GE healthcare) size exclusion chromatography (SEC) column equilibrated with PBS buffer. The integrity of the sample was assessed by endotoxin measurement and SDS-PAGE under reducing and non-reducing conditions. A representative gel for VG68 is shown in FIG. 8. The final protein concentrations were 1.0 mg/ml for anti-TRGV9/anti-CD33 and 1.0 mg/mL for ANTI-TRGV9/Null. The final EU levels of ANTI-TRGV9/anti-CD33 and ANTI-TRGV9/Null based on these were <3.0 EU/mg.

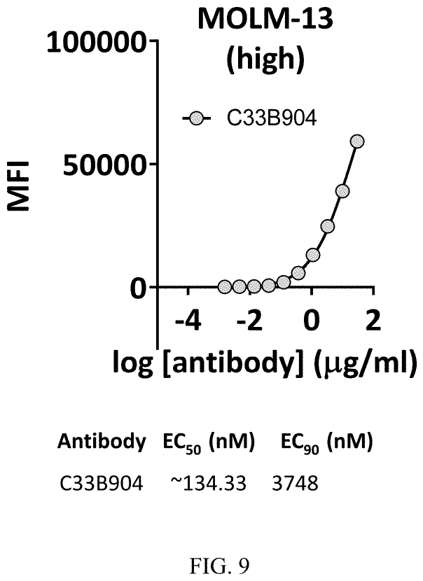

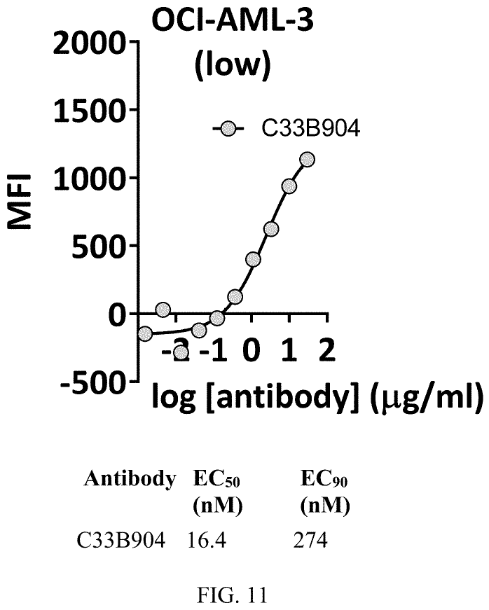

The binding activity of anti-CD33 antibody on target cell lines was assessed. The binding of anti-CD33 clone C33B904 to a panel CD33+ cell lines were measured by FACS. The EC50 and EC90 were calculated for MOLM-13 (FIG. 9), Kasumi-1 (FIG. 10) and OCI-AML-3 (FIG. 11).

### Example 4.3: Characterization of Vδ2+ (γδ) T Cells and Pan T Cells

Zoledronic acid was used to selectively expand Vγ9⁺γδ T cells from whole PBMCs. The PBMCs were isolated from whole fresh PBMCs using the EasySep™ Human γδT cell isolation kit (Stem cell Technologies; Vancouver, Calif.) according to the manufacturer's instructions. Isolated PBMCs were cultured in RPMI-10 (RPMI supplemented with 10% FBS, lx Pen/Strep) medium with recombinant human IL-2 (rhIL-2) to a final concentration of 1000 IU/mL, recombinant human IL-15 (rhIL-15) to a final concentration of 10 ng/mL, and Zoledronic acid to a final concentration of 5 μM for 14 days.

### Example 4.4: Evaluation of Binding and Cytotoxic Properties of the Anti-TRGV9/Anti-CD33 Bispecific Antibody Using Kasumi-3 Cells and Human γδ T Cells

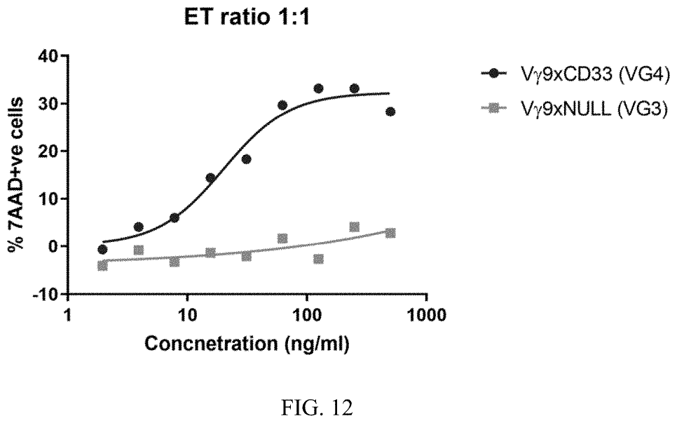

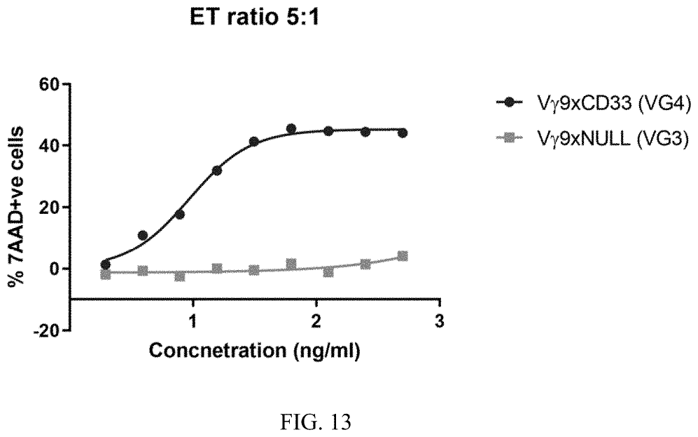

FIGS. 12-13 shows that the anti-TRGV9/anti-CD33 bispecific antibody mediates γδ T cell cytotoxicity against CD33 expressing Kasumi-3 cells in vitro. Enriched γδ T cells (Effectors), isolated from PBMCs cultured with Zoledronic acid+IL-2+IL-15 for 12 days, were co-cultured with CFSE labelled Kasumi-3 cells (Targets) at 1:1 and 5:1 E:T ratios in the presence of various concentrations of the bispecific antibody for 24 hours. Dose response curves show anti-TRGV9/anti-CD33 and anti-TRGV9/anti-NULL bispecific mediated γδ T cell cytotoxicity against CD33 expressing kasumi-3 cells in a dose dependent manner at 1:1 (FIG. 12) and 5:1 (FIG. 13) E:T ratios. Cytotoxicity values represented here were subtracted of basal cytotoxicity value observed in the absence of bispecific antibody. EC₅₀ values were calculated as described in methods. Representative data shown here are from a single experiment.

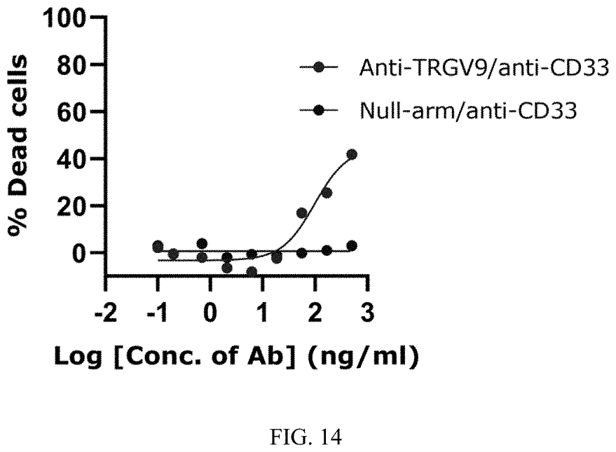

FIG. 14 shows that the anti-TRGV9/anti-CD33 bispecific antibody mediates) γδ T cell cytotoxicity (from whole PBMCs) against CD33 expressing MOLM-13 cells in vitro. Healthy donor derived PBMCs (Effectors), cultured with Zoledronic acid+IL-2+IL-15 for 12 days, were co-cultured with CFSE labelled MOLM-13 cells (Targets) at 1:1 E:T ratios in the presence of various concentrations of the bispecific antibody for 24 hours. Dose response curves in FIG. 14 show anti-TRGV9/anti-CD33 and anti-TRGV9/anti-NULL bispecific mediated γδ T cell cytotoxicity against CD33 expressing kasumi-3 cells in a dose dependent manner. Cytotoxicity values represented there were subtracted of basal cytotoxicity value observed in the absence of bispecific antibody. EC₅₀ values were calculated as described in methods. Representative data shown here are from a single experiment.

### Example 5: Humanization of Anti-Trgv9 Clone 7A5

Mouse anti-human Vγ9 clone LP7A5 (7A5) binds to the antigen (Vγ9-Vδ2 fused to human Fc) with a KD of 1.9 nM. Humanization of murine 7A5 was performed following the approach outlined by Singh et al., mAbs J, 2015. Based on sequence conservation, the heavy chain germline IGHV1-8*01 was chosen for framework adaption. Three somatic hypermutation sites in the heavy chain were chosen for binary library design. A potential Iso-Asp isomerization site (DG motif) in the CDR-H2 was also included in the design to mitigate this potential liability. For light chain frame adaption, IGKV4-1*01 was chosen as the closest homologous human germline. Owing to high sequence homology, only one position (Asn22) was included in the library design. The variants were cloned and expressed in E. coli. The supernatants were screened in single point ELISA and the periplasmic preparation was used for dose response. A mouse/human chimeric 7A5 Fab was used as parental control. Clone 7A5_17 (7A5_var17) maintained the binding activity similar to murine 7A5 and was converted to IgG for additional profiling. The EC₅₀ for primary cell binding for clone 7A5_17 and 7A5 were 200 pM and 159 pM.

The sequences obtained are shown in Tables 14-17. The three VH CDR and three VL CDR sequences of the humanized anti-human TRGV9 clone 7A5_var17 are shown in Table 14 (two versions, depending on CDR type, are provided); and the VH and VL sequences of the humanized anti-human TRGV9 clone 7A5_var17 are shown in Table 16 (SEQ ID NOs:65 and 66, respectively). The three VH CDR and three VL CDR sequences of the humanized anti-human TRGV9 clone 7A5_var29 are shown in Table 15 (two versions, depending on CDR type, are provided); and the VH and VL sequences of the humanized anti-human TRGV9 clone 7A5_var29 are shown in Table 17 (SEQ ID NOs:67 and 68, respectively).

TABLE 14
CDR sequences of humanized anti-human TRGV9 clone 7A5_var 17.

| | | SEQ | | SEQ | | SEQ |
|---|---|---|---|---|---|---|
| | | ID | | ID | | ID |
| mAb ID | HCDR1 | NO: | HCDR2 | NO: | HCDR3 | NO: |
| 7A5_var17 | GFTFTDHY | 60 | IYPGSGNT | 61 | ARNYGDYTIDF | 62 |
| (CDR v.1) | | | | | | |
| 7A5_var17 | DHYIN | 1 | QIYPGSGN | 76 | NYGDYTIDF | 3 |
| (CDR v.2) | | | TYYNQKFKG | | | |
| | | SEQ | | SEQ | | SEQ |
| | | ID | | ID | | ID |
| mAb ID | LCDR1 | NO: | LCDR2 | NO: | LCDR3 | NO: |
| 7A5_var17 | QSVLYSSNNKNY | 63 | WAS | 64 | QQYYRYHT | 6 |
| (CDR v.1) | | | | | | |
| 7A5_var17 | KSSQSVLY | 77 | WASTRES | 5 | QQYYRYHT | 6 |
| (CDR v.2) | SSNNKNYLA | | | | | |

TABLE 15
CDR sequences of humanized anti-human TRGV9 clone 7A5_var 29.

| | | SEQ | | SEQ | | SEQ |
|---|---|---|---|---|---|---|
| | | ID | | ID | | ID |
| mAb ID | HCDR1 | NO: | HCDR2 | NO: | HCDR3 | NO: |
| 7A5_var29 | GFTFTDHY | 60 | IYPGSGNT | 61 | ARNYGD | 62 |
| (CDR v.1) | | | | | YTIDF | |
| 7A5_var29 | DHYIN | 1 | QIYPGSGNT | 76 | NYGDYTIDF | 3 |
| (CDR v.2) | | | YYNQKFKG | | | |
| | | SEQ | | SEQ | | SEQ |
| | | ID | | ID | | ID |
| mAb ID | LCDR1 | NO: | LCDR2 | NO: | LCDR3 | NO: |
| 7A5_var29 | QSVLYSSNNKNY | 63 | WAS | 64 | QQYYRYHT | 6 |
| (CDR v.1) | | | | | | |
| 7A5_var29 | KSSQSVLYS | 77 | WASTRES | 5 | QQYYRYHT | 6 |
| (CDR v.2) | SNNKNYLA | | | | | |

TABLE 16
Heavy chain and light chain V-region sequences of humanized anti-
human TRGV9 clone 7A5_var17.

| | | SEQ |
|---|---|---|
| | | ID |
| mAb ID | Heavy Chain V-region Amino Acid Sequence | NO: |
| 7A5_var17 | QVQLVQSGAE VKKPGASVKV SCKASGFTFT DHYINWVRQA TGQGLEWMGQ | 65 |
| | IYPGSGNTYY NQKFKGRVTM TRDTSISTAY MELSSLRSED TAVYYCARNY | |
| | GDYTIDFWGQ GTSVTVSS | |
| | | SEQ |
| | | ID |
| mAb ID | Light Chain V-region Amino Acid Sequence | NO: |
| 7A5_var17 | DIVMTQSPDS LAVSLGERAT INCKSSQSVL YSSNNKNYLA WYQQKPGQPP | 66 |
| | KLLIYWASTR ESGVPDRFSG SGSGTDFTLT ISSLQAEDVA VYYCQQYYRY | |
| | HTFGTGTKLE IK | |

TABLE 17
Heavy chain and light chain V-region sequences of humanized anti-
human TRGV9 clone 7A5_var29.

| | | SEQ |
|---|---|---|
| | | ID |
| mAb ID | Heavy Chain V-region Amino Acid Sequence | NO: |
| 7A5_var29 | QVQLVQSGAE VKKPGASVKV SCKASGFTFT DHYINWVRQA TGQGLEWMGQ | 67 |
| | IYPGSGNTYY NQKFKGRVTM TRNTSISTAY MELSSLRSED TAVYYCARNY | |
| | GDYTIDFWGQ GTSVTVSS | |
| | | SEQ |
| | | ID |
| mAb ID | Light Chain V-region Amino Acid Sequence | NO: |
| 7A5_var29 | DIVMTQSPDS LAVSLGERAT ISCKSSQSVL YSSNNKNYLA WYQQKPGQPP | 68 |
| | KLLIYWASTR ESGVPDRFSG SGSGTDFTLT ISSLQAEDVA VYYCQQYYRY | |
| | HTFGTGTKLE IK | |

### Example 6—Multispecific Antibodies that Bind Trgv9 and TRBC1

T cell acute lymphoblastic leukemia (T-ALL) are aggressive neoplasms characterized by the proliferation and accumulation in blood, bone marrow and lymphoid organs of T cell precursors abnormally arrested in differentiation. Current first-line chemotherapy regimens provide overall survival rates of approximately 85-90% in children and about 50% in adults (Pui et al. J. Clin. Oncol. Off. J. Am. Soc. Clin. Oncol. 2015; 33 (27): 2938-2948; Litzow et al. Blood. 2015; 126 (7): 833-841). T-ALL represent a heterogeneous group of malignancies classified into different molecular subtypes on the basis of aberrant expression of specific driver oncogenic transcription factors and global transcriptomic signatures (Belver et al. Nat. Rev. Cancer. 2016; 16 (8): 494-507; Nat. Genet. 2017; 49 (8):1211-1218). T-cells are the most abundant (˜75% of blood lymphocytes) and potent immune killer cells. The role of effector T-cells in the anti-cancer immune response is strongly supported by in vitro studies and the observation that a high infiltration of CD8+ T cells in several types of cancers correlates with a favorable clinical prognostic.

Vγ9Vδ2 T lymphocytes, a major γ/δ T cell subset in humans, recognize phosphoantigens, certain tumor cells, and cells treated with aminobisphosphonates. This cell population displays cytolytic activity against various tumor cells. The γ/δ TCR is a heterodimeric TCR complex composed of covalently bound γ and δ chains involved in antigen recognition and the non-covalently associated monomorphic proteins CD3δ, γ, ε, and ξ chains. The Vγ9 TCR is a variant of the TCR γ chain expressed on a subset of γ/δ T cells.

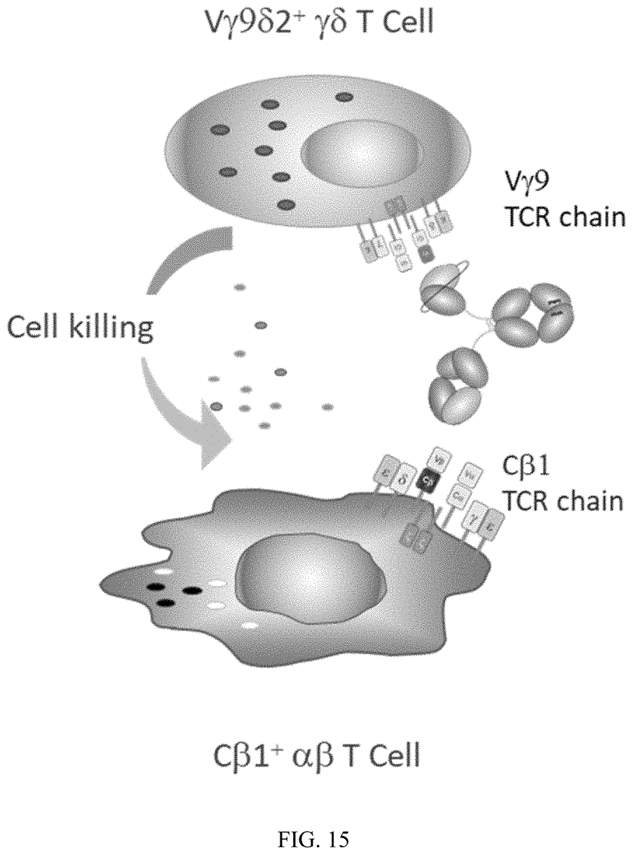

Examples 6.1-6.6 are based on the premise that γδ T cells, which mainly express heterodimers of TRGV9 and Vδ2 chains demonstrate potent anti-tumor functions. These cells express TCR-TRGV9 and the majority, if not all, of these cells exhibit efficient cytotoxicity of tumor target cells. This ability is then harnessed using bispecific antibodies constructed such that one arm binds to the TRGV9 structure and the other arm binds to a tumor-associated antigen (TRBC1) expressed by the tumor cells (e.g., T cell lymphomas). Thus, the bispecific antibody bridges the effector and target cells together-resulting in tumor cell killing. This mechanism of action is described in the schematic outlined in FIG. 15.

The subsequent examples can be divided into the following categories: (1) Generation and characterization of bispecific antibodies capable of binding to the TRGV9 arm expressed on γδ T cells and TRBC1 on αβ T cells (Examples 6.1, 6.2, and 6.3); and (2) Evidence for bispecific antibody binding and bispecific antibody-enabled target cell killing by γδ T cells expanded in vitro (Example 6.4 and 6.5).

γδ T cell stimulation and expansion was performed. Expansion of Vγ9-Vδ2 T cells was carried out by treating PBMCs in complete RPMI media containing rhIL-2 (1000 IU/mL), rhIL-15 (10 ng/mL) and zoledronic acid (5 μM) for 14 days.

### Example 6.1: Production and De Novo Sequencing of Anti-TRGV9 Mab

The mouse IgG1 anti-human T cell receptor anti-TRGV9 clone 7A5 was soured commercially. Sample preparation and LC-MS/MS analysis were performed by Lake Pharma (San Carlos, Calif.). The sample was reduced and alkylated, divided into seven aliquots, and proteolytically digested with Trypsin/LysC, Chymotrypsin, LysC, Pepsin, and AspN, Elastase, and Proteinase K enzymes. Resulting peptides were desalted using a ZipTip C18 Pipette Tips and separated on-line using reverse phase chromatography. Mass spectrometry was performed on Thermo Q-Exactive spectrometer using HCD fragmentation. MS data sets were analyzed using PEAKS software by matching de novo sequence tags to an IMGT-based antibody sequences database. Gaps in the sequence were assigned using Contig sequence assembly of de novo identified peptides. All CDRs and hyper-mutations were confirmed by inspecting the MS/MS spectra.

The three VH CDR and three VL CDR sequences of the mouse anti-human TRGV9 clone 7A5 (LP7A5_1) are previously shown in Table 1 (SEQ ID NOs:1-6, respectively); and the VH and VL sequences of the mouse anti-human TRGV9 clone 7A5 (LP7A5_1) are previously shown in Table 5 (SEQ ID NOs:7 and 8, respectively).

### Example 6.2: Production and De Novo Sequencing of Anti-TRBC1 Mab

The mouse IgG2a monoclonal anti-human T cell receptor anti-TRBC1 clone JOVI-1 was commercially sourced. The sequences of JOVI-1 were obtained using a similar procedure as described above for the anti-TRGV9 clone 7A5.

The three VH CDR and three VL CDR sequences of anti-human T cell receptor anti-TRBC1 clone JOVI-1 are shown in Table 18 (SEQ ID NOs:49-54, respectively); and the VH and VL sequences of the anti-human T cell receptor anti-TRBC1 clone JOVI-1 are shown in Table 19 (SEQ ID NOs:55 and 56, respectively).

TABLE 18
CDR sequences of anti-TRBC1 mAb.

| | | SEQ | | SEQ | | SEQ |
|---|---|---|---|---|---|---|
| | | ID | | ID | | ID |
| mAb ID | HCDR1 | NO: | HCDR2 | NO: | HCDR3 | NO: |
| mJOVI-1 | GYTFTGYV | 49 | INPYNDDI | 50 | ARGAGYNFD | 51 |
| | | | | | GAYRFFDF | |
| | | SEQ | | SEQ | | SEQ |
| | | ID | | ID | | ID |
| mAb ID | LCDR1 | NO: | LCDR2 | NO: | LCDR3 | NO: |
| mJOVI-1 | QRLVHSNGNTY | 52 | RVS | 53 | SQSTHVPYT | 54 |

TABLE 19
Heavy chain and light chain sequences of anti-
TRBC1 mAb.

| | | SEQ |
|---|---|---|
| | | ID |
| mAb ID | Heavy Chain Amino Acid Sequence | NO: |
| mJOVI-1 | EVRLQQSGPDLIKPGASVKMSCKASGYTFTGYVMHWV | 55 |
| | KQRPGQGLEWIGFINPYNDDIQSNERFRGKATLTSDK | |
| | SSTTAYMELSSLTSEDSAVYYCARGAGYNFDGAYRFF | |
| | DFWGQGTTLTVSS | |
| | | SEQ |
| | | ID |
| mAb ID | Light Chain Amino Acid Sequence | NO: |
| mJOVI-1 | DVVMTQSPLSLPVSLGDQASISCRSSQRLVHSNGNTY | 56 |
| | LHWYLQKPGQSPKLLIYRVSNRFPGVPDRFSGSGSGT | |
| | DFTLKISRVEAEDLGIYFCSQSTHVPYTFGGGTKLEIK | |

### Example 6.3: Preparation of Anti-TRGV9/Anti-TRBC1 Bispecific Antibodies

The variable region sequence of clone 7A5 (anti-TRGV9 antibody) and clone JOVI-1 (anti-TRBC1 antibody) (HCDRs and LCDRs in Table 18, HC and LC in Table 19) were used to generate a bispecific antibody to be tested for T cell re-directed killing of acute myeloid leukemia (AML) cells. The bispecific antibodies VG4 (anti-TRGV9×TRBC1) and VG3 (anti-TRGV9×Null) were produced as full-length antibodies in the knob-into-hole format as human IgG4. Nucleic acid sequences encoding variable regions were sub-cloned into a custom mammalian expression vectors containing constant region of human IgG4 expression cassettes using standard PCR restriction enzyme based standard cloning techniques, and sequenced verified. The bispecific antibodies were expressed by transient transfection in a Chinese hamster ovary (CHO) cell line. The sequences of the bispecific antibodies expressed in the CHO cells are shown in Table 20 below. Individual heavy and light chain antibody sequences are shown in Table 21 below

TABLE 20
Sequences of antibodies expressed in CHO cells

| mAb ID | ‘Knob’ arm and ‘hole’ arm amino acid sequence | | |
|---|---|---|---|
| Anti- | Heavy | MAWVWTLLFLMAAAQSIQAEVQLQQSGAELARPGASVKLSCKASGFTFTDHY | SEQ ID |
| TRGV9_ | Chain A | INWVKQRTGQGLEWIGQIYPGDGNTYYNQKFKGKATLTADKSSSTAYMQLSS | NO: |
| 7A5_1 | | LTSEDSAVYFCAPNYGDYTIDFWGQGTSVTVSSASTKGPSVFPLAPSSKSTS | 78 |
| (half- | | GGTAALGCLVKDYFPEPVTVSWNSGALTSGVHTFPAVLQSSGLYSLSSVVTV | |
| mAb) | | PSSSLGTQTYICNVNHKPSNTKVDKKVEPKSCDKTHTCPPCPAPEAAGGPSV | |
| | | FLFPPKPKDTLMISRTPEVTCVVVSVSHEDPEVKFNWYVDGVEVHNAKTKPR | |
| | | EEQYNSTYRVVSVLTVLHQDWLNGKEYKCKVSNKALPAPIEKTISKAKGQPR | |
| | | EPQVYVYPPSREEMTKNQVSLTCLVKGFYPSDIAVEWESNGQPENNYKTTPP | |
| | | VLDSDGSFALVSKLTVDKSRWQQGNVFSCSVMHEALHNRFTQKSLSLSPGK | |
| | Light | MAWVWTLLFLMAAAQSIQADIVMSQSPSSLAVSVGEKVTMSCKSSQSLLYSS | SEQ ID |
| | Chain | NQKNYLAWYQQKPGQSPKLLIYWASTRESGVPDRFTGSGSGTDFTLTISSVK | NO: |
| | | AEDLAVYYCQQYYRYHTFGTGTKLEIKRTVAAPSVFIFPPSDEQLKSGTASV | 79 |
| | | VCLLNNFYPREAKVQWKVDNALQSGNSQESVTEQDSKDSTYSLSSTLTLSKA | |
| | | DYEKHKVYACEVTHQGLSSPVTKSFNRGEC | |
| Anti- | Heavy | MAWVWTLLFLMAAAQSIQAQVQLVQSGAEVKKPGASVKVSCKASGFTFTDHY | SEQ ID |
| TRGV9_ | Chain A | INWVRQATGQGLEWMGQIYPGSGNTYYNQKFKGRVTMTRDTSISTAYMELSS | NO: |
| 7A5_var | | LRSEDTAVYYCARNYGDYTIDFWGQGTSVTVSSASTKGPSVFPLAPSSKSTS | 80 |
| 17 | | GGTAALGCLVKDYFPEPVTVSWNSGALTSGVHTFPAVLQSSGLYSLSSVVTV | |
| | | PSSSLGTQTYICNVNHKPSNTKVDKKVEPKSCDKTHTCPPCPAPEAAGGPSV | |
| | | FLFPPKPKDTLMISRTPEVTCVVVSVSHEDPEVKFNWYVDGVEVHNAKTKPR | |
| | | EEQYNSTYRVVSVLTVLHQDWLNGKEYKCKVSNKALPAPIEKTISKAKGQPR | |
| | | EPQVYVYPPSREEMTKNQVSLTCLVKGFYPSDIAVEWESNGQPENNYKTTPP | |
| | | VLDSDGSFALVSKLTVDKSRWQQGNVFSCSVMHEALHNRFTQKSLSLSPGK | |
| | Light | MAWVWTLLFLMAAAQSIQADIVMTQSPDSLAVSLGERATINCKSSQSVLYSS | SEQ ID |
| | Chain | NNKNYLAWYQQKPGQPPKLLIYWASTRESGVPDRFSGSGSGTDFTLTISSLQ | NO: |
| | | AEDVAVYYCQQYYRYHTFGTGTKLEIKRTVAAPSVFIFPPSDEQLKSGTASV | 81 |
| | | VCLLNNFYPREAKVQWKVDNALQSGNSQESVTEQDSKDSTYSLSSTLTLSKA | |
| | | DYEKHKVYACEVTHQGLSSPVTKSFNRGEC | |
| Anti- | Heavy | MAWVWTLLFLMAAAQSIQAQVQLVQSGAEVKKPGASVKVSCKASGFTFTDHY | SEQ ID |
| TRGV9_ | Chain A | INWVRQATGQGLEWMGQIYPGSGNTYYNQKFKGRVTMTRNTSISTAYMELSS | NO: |
| 7A5_var | | LRSEDTAVYYCARNYGDYTIDFWGQGTSVTVSSASTKGPSVFPLAPSSKSTS | 82 |
| 29 | | GGTAALGCLVKDYFPEPVTVSWNSGALTSGVHTFPAVLQSSGLYSLSSVVTV | |
| | | PSSSLGTQTYICNVNHKPSNTKVDKKVEPKSCDKTHTCPPCPAPEAAGGPSV | |
| | | FLFPPKPKDTLMISRTPEVTCVVVSVSHEDPEVKFNWYVDGVEVHNAKTKPR | |
| | | EEQYNSTYRVVSVLTVLHQDWLNGKEYKCKVSNKALPAPIEKTISKAKGQPR | |
| | | EPQVYVYPPSREEMTKNQVSLTCLVKGFYPSDIAVEWESNGQPENNYKTTPP | |
| | | VLDSDGSFALVSKLTVDKSRWQQGNVFSCSVMHEALHNRFTQKSLSLSPGK | |
| | Light | MAWVWTLLFLMAAAQSIQADIVMTQSPDSLAVSLGERATISCKSSQSVLYSS | SEQ ID |
| | Chain | NNKNYLAWYQQKPGQPPKLLIYWASTRESGVPDRFSGSGSGTDFTLTISSLQ | NO: |
| | | AEDVAVYYCQQYYRYHTFGTGTKLEIKRTVAAPSVFIFPPSDEQLKSGTASV | 83 |
| | | VCLLNNFYPREAKVQWKVDNALQSGNSQESVTEQDSKDSTYSLSSTLTLSKA | |
| | | DYEKHKVYACEVTHQGLSSPVTKSFNRGEC | |
| Anti- | Heavy | MAWVWTLLFLMAAAQSIQAEVRLQQSGPDLIKPGASVKMSCKASGYTFTGYV | SEQ ID |
| TRBC1 | Chain B | MHWVKQRPGQGLEWIGFINPYNDDIQSNERFRGKATLTSDKSSTTAYMELSS | NO: |
| (half- | | LTSEDSAVYYCARGAGYNFDGAYRFFDFWGQGTTLTVSSASTKGPSVFPLAP | 84 |
| mAb) | | SSKSTSGGTAALGCLVKDYFPEPVTVSWNSGALTSGVHTFPAVLQSSGLYSL | |
| | | SSVVTVPSSSLGTQTYICNVNHKPSNTKVDKKVEPKSCDKTHTCPPCPAPEA | |
| | | AGGPSVFLFPPKPKDTLMISRTPEVTCVVVSVSHEDPEVKFNWYVDGVEVHN | |
| | | AKTKPREEQYNSTYRVVSVLTVLHQDWLNGKEYKCKVSNKALPAPIEKTISK | |
| | | AKGQPREPQVYVLPPSREEMTKNQVSLLCLVKGFYPSDIAVEWESNGQPENN | |
| | | YLTWPPVLDSDGSFFLYSKLTVDKSRWQQGNVFSCSVMHEALHNRFTQKSLS | |
| | | LSPGK | |
| | Light | MAWVWTLLFLMAAAQSIQADVVMTQSPLSLPVSLGDQASISCRSSQRLVHSN | SEQ ID |
| | Chain | GNTYLHWYLQKPGQSPKLLIYRVSNRFPGVPDRFSGSGSGTDFTLKISRVEA | NO: |
| | | EDLGIYFCSQSTHVPYTFGGGTKLEIKRTVAAPSVFIFPPSDEQLKSGTASV | 85 |
| | | VCLLNNFYPREAKVQWKVDNALQSGNSQESVTEQDSKDSTYSLSSTLTLSKA | |
| | | DYEKHKVYACEVTHQGLSSPVTKSFNRGEC | |
| Anti- | Heavy | MAWVWTLLFLMAAAQSIQAQITLKESGPTLVKPTQTLTLTCTFSGFSLSTSG | SEQ ID |
| RSV | Chain B | MGVSWIRQPPGKALEWLAHIYWDDDKRYNPSLKSRLTITKDTSKNQVVLTMT | NO: |
| (half- | | NMDPVDTATYYCARLYGFTYGFAYWGQGTLVTVSSASTKGPSVFPLAPSSKS | 86 |
| mAb) | | TSGGTAALGCLVKDYFPEPVTVSWNSGALTSGVHTFPAVLQSSGLYSLSSVV | |
| | | TVPSSSLGTQTYICNVNHKPSNTKVDKKVEPKSCDKTHTCPPCPAPEAAGGP | |
| | | SVFLFPPKPKDTLMISRTPEVTCVVVSVSHEDPEVKFNWYVDGVEVHNAKTK | |
| | | PREEQYNSTYRVVSVLTVLHQDWLNGKEYKCKVSNKALPAPIEKTISKAKGQ | |
| | | PREPQVYVLPPSREEMTKNQVSLLCLVKGFYPSDIAVEWESNGQPENNYLTW | |
| | | PPVLDSDGSFFLYSKLTVDKSRWQQGNVFSCSVMHEALHNHYTQKSLSLSPG | |
| | | K | |
| | Light | METHSQVFVYMLLWLSGVEGDIVMTQSPDSLAVSLGERATINCRASQSVDYN | SEQ ID |
| | Chain | GISYMHWYQQKPGQPPKLLIYAASNPESGVPDRFSGSGSGTDFTLTISSLQA | NO: |
| | | EDVAVYYCQQIIEDPWTFGQGTKVEIKRTVAAPSVFIFPPSDEQLKSGTASV | 87 |
| | | VCLLNNFYPREAKVQWKVDNALQSGNSQESVTEQDSKDSTYSLSSTLTLSKA | |
| | | DYEKHKVYACEVTHQGLSSPVTKSFNRGEC | |

scFv Sequences

| | | | |
|---|---|---|---|
| Anti- | Heavy | MAWVWTLLFLMAAAQSIQADIVMTQSPDSLAVSLGERATINCKSSQSVLYSS | SEQ ID |
| TRGV9_ | Chain A | NNKNYLAWYQQKPGQPPKLLIYWASTRESGVPDRFSGSGSGTDFTLTISSLQ | NO: |
| 7A5_var | | AEDVAVYYCQQYYRYHTFGTGTKLEIKGGSEGKSSGSGSESKSTGGSQVQLV | 70 |
| 17-scFv | | QSGAEVKKPGASVKVSCKASGFTFTDHYINWVRQATGQGLEWMGQIYPGSGN | |
| | | TYYNQKFKGRVTMTRDTSISTAYMELSSLRSEDTAVYYCARNYGDYTIDFWG | |
| | | QGTSVTVSSEPKSSDKTHTCPPCPAPEAAGGPSVFLFPPKPKDTLMISRTPE | |
| | | VTCVVVSVSHEDPEVKFNWYVDGVEVHNAKTKPREEQYNSTYRVVSVLTVLH | |
| | | QDWLNGKEYKCKVSNKALPAPIEKTISKAKGQPREPQVYVYPPSREEMTKNQ | |
| | | VSLTCLVKGFYPSDIAVEWESNGQPENNYKTTPPVLDSDGSFALVSKLTVDK | |
| | | SRWQQGNVFSCSVMHEALHNHYTQKSLSLSPGK | |
| Anti- | Heavy | MAWVWTLLFLMAAAQSIQADIVMTQSPDSLAVSLGERATISCKSSQSVLYSS | SEQ ID |
| TRGV9_ | Chain A | NNKNYLAWYQQKPGQPPKLLIYWASTRESGVPDRFSGSGSGTDFTLTISSLQ | NO: |
| 7A5_var | | AEDVAVYYCQQYYRYHTFGTGTKLEIKGGSEGKSSGSGSESKSTGGSQVQLV | 73 |
| 29-scFv | | QSGAEVKKPGASVKVSCKASGFTFTDHYINWVRQATGQGLEWMGQIYPGSGN | |
| | | TYYNQKFKGRVTMTRNTSISTAYMELSSLRSEDTAVYYCARNYGDYTIDFWG | |
| | | QGTSVTVSSEPKSSDKTHTCPPCPAPEAAGGPSVFLFPPKPKDTLMISRTPE | |
| | | VTCVVVSVSHEDPEVKFNWYVDGVEVHNAKTKPREEQYNSTYRVVSVLTVLH | |
| | | QDWLNGKEYKCKVSNKALPAPIEKTISKAKGQPREPQVYVYPPSREEMTKNQ | |
| | | VSLTCLVKGFYPSDIAVEWESNGQPENNYKTTPPVLDSDGSFALVSKLTVDK | |
| | | SRWQQGNVFSCSVMHEALHNHYTQKSLSLSPGK | |
| Anti- | Heavy | MAWVWTLLFLMAAAQSIQADVVMTQSPLSLPVSLGDQASISCRSSQRLVHSN | SEQ ID |
| TRBC1- | Chain B | GNTYLHWYLQKPGQSPKLLIYRVSNRFPGVPDRFSGSGSGTDFTLKISRVEA | NO: |
| scFv | | EDLGIYFCSQSTHVPYTFGGGTKLEIKGGSEGKSSGSGSESKSTGGSEVRLQ | 57 |
| | | QSGPDLIKPGASVKMSCKASGYTFTGYVMHWVKQRPGQGLEWIGFINPYNDD | |
| | | IQSNERFRGKATLTSDKSSTTAYMELSSLTSEDSAVYYCARGAGYNFDGAYR | |
| | | FFDFWGQGTTLTVSSEPKSSDKTHTCPPCPAPEAAGGPSVFLFPPKPKDTLM | |
| | | ISRTPEVTCVVVSVSHEDPEVKFNWYVDGVEVHNAKTKPREEQYNSTYRVVS | |
| | | VLTVLHQDWLNGKEYKCKVSNKALPAPIEKTISKAKGQPREPQVYVLPPSRE | |
| | | EMTKNQVSLLCLVKGFYPSDIAVEWESNGQPENNYLTWPPVLDSDGSFFLYS | |
| | | KLTVDKSRWQQGNVFSCSVMHEALHNHYTQKSLSLSPGK | |
| Anti- | Heavy | MAWVWTLLFLMAAAQSIQADIQMTQSPSSLSASVGDRVTITCRASQSISSYL | SEQ ID |
| Null- | Chain B | NWYQQKPGCAPKLLIYAASSLQSGVPSRFSGSGSGTDFTLTISSLQPEDFAT | NO: |
| scFv | | YYCQQSYSTPLTFGQGTKVEIKGGGSGGSGGCPPCGGSGGEVQLLESGGGLV | 88 |
| | | QPGGSLRLSCAASGFTFSSYAMSWVRQAPGKGLEWVSAISGSGGSTYYADSV | |
| | | KGRFTISRDNSKNTLYLQMNSLRAEDTAVYYCAKYDGIYGELDFWGCGTLVT | |
| | | VSSEPKSSDKTHTCPPCPAPEAAGGPSVFLFPPKPKDTLMISRTPEVTCVVV | |
| | | SVSHEDPEVKFNWYVDGVEVHNAKTKPREEQYNSTYRVVSVLTVLHQDWLNG | |
| | | KEYKCKVSNKALPAPIEKTISKAKGQPREPQVYVLPPSREEMTKNQVSLLCL | |
| | | VKGFYPSDIAVEWESNGQPENNYLTWPPVLDSDGSFFLYSKLTVDKSRWQQG | |
| | | NVFSCSVMHEALHNHYTQKSLSLSPGK | |

TABLE 21
Anti-TRGV9 and Anti-TRBC1 Heavy and Light Chain Sequences

| Antibody | Heavy Chain | Light Chain |
|---|---|---|
| TRGV9_ | EVQLQQSGAELARPGASVKLSCKASGF | DIVMSQSPSSLAVSVGEKVTMSCKSSQSLL |
| 7A5_1 | TFTDHYINWVKQRTGQGLEWIGQIYPG | YSSNQKNYLAWYQQKPGQSPKLLIYWASTR |
| | DGNTYYNQKFKGKATLTADKSSSTAYM | ESGVPDRFTGSGSGTDFTLTISSVKAEDLA |
| | QLSSLTSEDSAVYFCAPNYGDYTIDFW | VYYCQQYYRYHTFGTGTKLEIKRTVAAPSV |
| | GQGTSVTVSSASTKGPSVFPLAPSSKS | FIFPPSDEQLKSGTASVVCLLNNFYPREAK |
| | TSGGTAALGCLVKDYFPEPVTVSWNSG | VQWKVDNALQSGNSQESVTEQDSKDSTYSL |
| | ALTSGVHTFPAVLQSSGLYSLSSVVTV | SSTLTLSKADYEKHKVYACEVTHQGLSSPV |
| | PSSSLGTQTYICNVNHKPSNTKVDKKV | TKSFNRGEC (SEQ ID NO: 24) |
| | EPKSCDKTHTCPPCPAPEAAGGPSVFL | |
| | FPPKPKDTLMISRTPEVTCVVVSVSHE | |
| | DPEVKFNWYVDGVEVHNAKTKPREEQY | |
| | NSTYRVVSVLTVLHQDWLNGKEYKCKV | |
| | SNKALPAPIEKTISKAKGQPREPQVYV | |
| | YPPSREEMTKNQVSLTCLVKGFYPSDI | |
| | AVEWESNGQPENNYKTTPPVLDSDGSF | |
| | ALVSKLTVDKSRWQQGNVFSCSVMHEA | |
| | LHNRFTQKSLSLSPGK (SEQ ID | |
| | NO: 69) | |
| TRGV9_ | QVQLVQSGAEVKKPGASVKVSCKASGF | DIVMTQSPDSLAVSLGERATINCKSSQSVL |
| var17 | TFTDHYINWVRQATGQGLEWMGQIYPG | YSSNNKNYLAWYQQKPGQPPKLLIYWASTR |
| | SGNTYYNQKFKGRVTMTRDTSISTAYM | ESGVPDRFSGSGSGTDFTLTISSLQAEDVA |
| | ELSSLRSEDTAVYYCARNYGDYTIDFW | VYYCQQYYRYHTFGTGTKLEIKRTVAAPSV |
| | GQGTSVTVSSASTKGPSVFPLAPSSKS | FIFPPSDEQLKSGTASVVCLLNNFYPREAK |
| | TSGGTAALGCLVKDYFPEPVTVSWNSG | VQWKVDNALQSGNSQESVTEQDSKDSTYSL |
| | ALTSGVHTFPAVLQSSGLYSLSSVVTV | SSTLTLSKADYEKHKVYACEVTHQGLSSPV |
| | PSSSLGTQTYICNVNHKPSNTKVDKKV | TKSFNRGEC (SEQ ID NO: 72) |
| | EPKSCDKTHTCPPCPAPEAAGGPSVFL | |
| | FPPKPKDTLMISRTPEVTCVVVSVSHE | |
| | DPEVKFNWYVDGVEVHNAKTKPREEQY | |
| | NSTYRVVSVLTVLHQDWLNGKEYKCKV | |
| | SNKALPAPIEKTISKAKGQPREPQVYV | |
| | YPPSREEMTKNQVSLTCLVKGFYPSDI | |
| | AVEWESNGQPENNYKTTPPVLDSDGSF | |
| | ALVSKLTVDKSRWQQGNVFSCSVMHEA | |
| | LHNRFTQKSLSLSPGK (SEQ ID | |
| | NO: 71) | |
| TRGV9_ | QVQLVQSGAEVKKPGASVKVSCKASGF | DIVMTQSPDSLAVSLGERATISCKSSQSVL |
| var29 | TFTDHYINWVRQATGQGLEWMGQIYPG | YSSNNKNYLAWYQQKPGQPPKLLIYWASTR |
| | SGNTYYNQKFKGRVTMTRNTSISTAYM | ESGVPDRFSGSGSGTDFTLTISSLQAEDVA |
| | ELSSLRSEDTAVYYCARNYGDYTIDFW | VYYCQQYYRYHTFGTGTKLEIKRTVAAPSV |
| | GQGTSVTVSSASTKGPSVFPLAPSSKS | FIFPPSDEQLKSGTASVVCLLNNFYPREAK |
| | TSGGTAALGCLVKDYFPEPVTVSWNSG | VQWKVDNALQSGNSQESVTEQDSKDSTYSL |
| | ALTSGVHTFPAVLQSSGLYSLSSVVTV | SSTLTLSKADYEKHKVYACEVTHQGLSSPV |
| | PSSSLGTQTYICNVNHKPSNTKVDKKV | TKSFNRGEC (SEQ ID NO: 75) |
| | EPKSCDKTHTCPPCPAPEAAGGPSVFL | |
| | FPPKPKDTLMISRTPEVTCVVVSVSHE | |
| | DPEVKFNWYVDGVEVHNAKTKPREEQY | |
| | NSTYRVVSVLTVLHQDWLNGKEYKCKV | |
| | SNKALPAPIEKTISKAKGQPREPQVYV | |
| | YPPSREEMTKNQVSLTCLVKGFYPSDI | |
| | AVEWESNGQPENNYKTTPPVLDSDGSF | |
| | ALVSKLTVDKSRWQQGNVFSCSVMHEA | |
| | LHNRFTQKSLSLSPGK (SEQ ID | |
| | NO: 74) | |
| TRBC1 | EVRLQQSGPDLIKPGASVKMSCKASGY | DVVMTQSPLSLPVSLGDQASISCRSSQRLV |
| | TFTGYVMHWVKQRPGQGLEWIGFINPY | HSNGNTYLHWYLQKPGQSPKLLIYRVSNRF |
| | NDDIQSNERFRGKATLTSDKSSTTAYM | PGVPDRFSGSGSGTDFTLKISRVEAEDLGI |
| | ELSSLTSEDSAVYYCARGAGYNFDGAY | YFCSQSTHVPYTFGGGTKLEIKRTVAAPSV |
| | RFFDFWGQGTTLTVSSASTKGPSVFPL | FIFPPSDEQLKSGTASVVCLLNNFYPREAK |
| | APSSKSTSGGTAALGCLVKDYFPEPVT | VQWKVDNALQSGNSQESVTEQDSKDSTYSL |
| | VSWNSGALTSGVHTFPAVLQSSGLYSL | SSTLTLSKADYEKHKVYACEVTHQGLSSPV |
| | SSVVTVPSSSLGTQTYICNVNHKPSNT | TKSFNRGEC (SEQ ID NO: 59) |
| | KVDKKVEPKSCDKTHTCPPCPAPEAAG | |
| | GPSVFLFPPKPKDTLMISRTPEVTCVV | |
| | VSVSHEDPEVKFNWYVDGVEVHNAKTK | |
| | PREEQYNSTYRVVSVLTVLHQDWLNGK | |
| | EYKCKVSNKALPAPIEKTISKAKGQPR | |
| | EPQVYVLPPSREEMTKNQVSLLCLVKG | |
| | FYPSDIAVEWESNGQPENNYLTWPPVL | |
| | DSDGSFFLYSKLTVDKSRWQQGNVFSC | |
| | SVMHEALHNRFTQKSLSLSPGK (SEQ | |
| | ID NO: 58) | |

The antibodies were initially purified by Mab Select SuRe Protein A column (GE Healthcare). The column was equilibrated with PBS pH 7.2 and loaded with fermentation supernatant at a flow rate of 2 mL/min. After loading, the column was washed with 4 column volumes of PBS followed by elution in 30 mM sodium acetate, pH 3.5. Fractions containing protein peaks as monitored by absorbance at 280 nm were pooled and neutralized to pH 5.0 by adding 1% 3 M sodium acetate pH 9.0. The bispecific mAbs were further purified on a preparative Superdex 200 10/300 GL (GE healthcare) size exclusion chromatography (SEC) column equilibrated with PBS buffer. The integrity of the sample was assessed by endotoxin measurement and SDS-PAGE under reducing and non-reducing conditions. The final protein concentrations were 1.0 mg/ml for anti-TRGV9/anti-TRBC1 and 1.0 mg/mL for ANTI-TRGV9/Null. The final EU levels of ANTI-TRGV9/anti-TRBC1 and ANTI-TRGV9/Null based on these were <3.0 EU/mg.

### Example 6.4: Binding Activity of Anti-TRBC1 and Anti-TRGV9 Antibodies on Target Cell Lines

Binding of antibodies to TRBC1 expressing cell line and γδ T cells was carried out by flow cytometry. Briefly, 50,000 target cells or γδ T cells were incubated at 4° C. for 45 minutes with serial dilutions of various antibodies. After washing with wash buffer (PBS+2% FBS), antibody bound to cell surface was detected by incubating the cells with PE labelled mouse anti human IgG1 secondary antibody (Southern Biotech, Birmingham, Ala.) for 30 minutes at 4° C. Cells were washed with wash buffer (PBS+2% FBS) and the fluorescence of stained cells was measured on Novocyte flow cytometer. Cells were visualized on forward and sideward scatter and doublets were excluded. No secondary antibody control was used to establish background fluorescence and to gate on specific population. Background value was subtracted from main samples to get specific binding value.

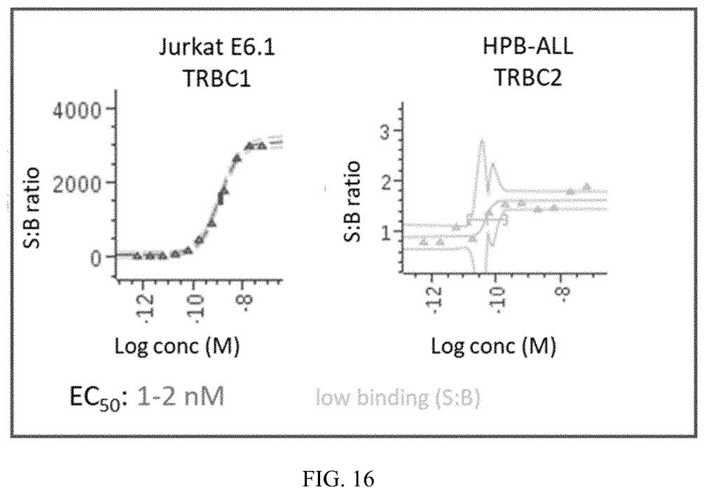

As shown in FIG. 16, the EC50 for binding of anti-TRBC1 antibodies (JOVI-1 mIgG2a, TRB1B1) to TRBC1 expressing Jurkat cell lines was ˜1 to 2 nM. TRB1B1 did not show any significant binding to HPB-ALL cell line that endogenously expresses TRBC2 TCR.

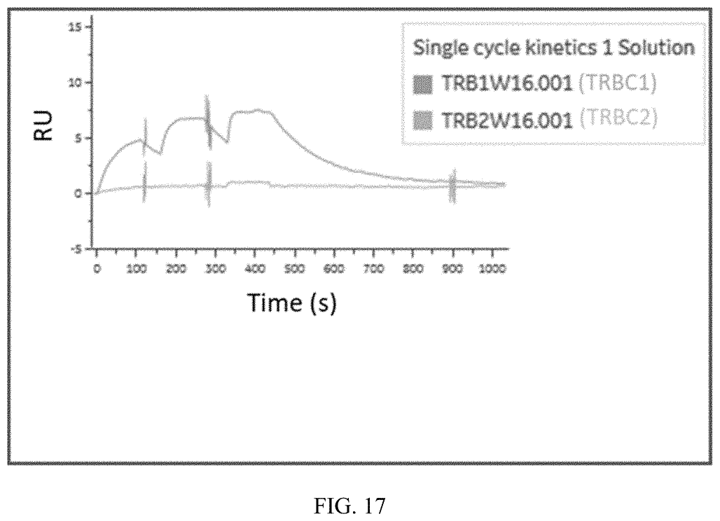

Additional surface plasmon resonance (SPR) experiments were used to determine specific binding of anti-TRBC1 mAb to TCR TRBC1. Briefly, SPR experiments were carried out in HBSP buffer at 25° C. The experimental set up was following: Goat anti-murine Fc surface was immobilized on a sensor chip, and binding was tested by capturing the mouse anti-human TRBC1 clone JOVI-1 mAb at different densities. The recombinant TRBC1-TCR protein (TRBC1W16) and TRBC2-TCR protein (TRBC2W16) were used as analyte to bind in solution in a single cycle kinetics. Raw binding data were processed by double referencing, e.g., interspot on an empty chip surface. As shown in FIG. 17, the anti-TRBC1 antibodies (JOVI-1 mIgG2a, TRB1B1) specifically bound to recombinant T cell receptor comprising of TRBC1 constant domain (TRB1W16). TRB1B1 did not show any significant binding to a recombinant T cell receptor with TRBC2 constant domain (TRB2W16).

### Example 6.5: Evaluation of Binding and Cytotoxic Properties of the Anti-TRGV9/Anti-TRBC1 Bispecific Antibody Using Jurkat Cells and Human γδ T Cells

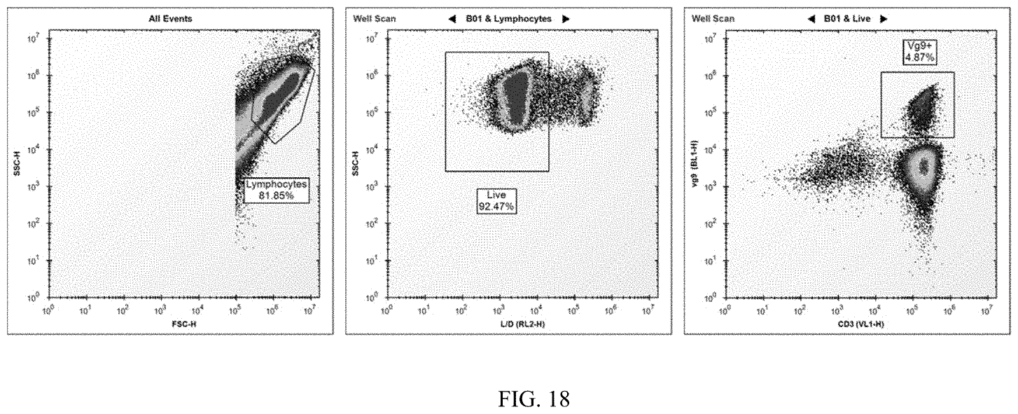

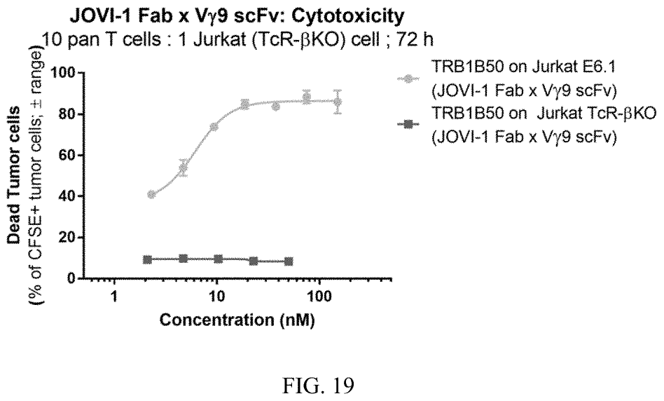

FIG. 18 shows the phenotyping of Vγ9+ cells (TRB1B50) from a healthy donor of used for cytotoxicity studies of a JOVI-1×Vγ9 bispecific. FIG. 19 shows that the anti-TRGV9/anti-TRBC1 bispecific antibody mediates γδ T cell (TRB150) cytotoxicity against TRBC1-expressing Jurkat cells in vitro. Enriched γδ T cells (Effectors), isolated from PBMCs cultured with Zoledronic acid+IL-2+IL-15 for 12 days, were co-cultured with CFSE labelled Jurkat cells (Targets) at 0.5:1 to 10:1 E:T ratios in the presence of various concentrations of the bispecific antibody for 24 to 72 hours. Dose response curves show an anti-TRGV9/anti-TRBC1 (JOVI-1 Fab×Vγ9 scFv) bispecific mediated γδ T cell cytotoxicity against TRBC1-expressing Jurkat cells in a dose dependent manner, as compared to Jurkat cells having a knock out of the TCR β chain. Cytotoxicity values represented here were subtracted of basal cytotoxicity value observed in the absence of bispecific antibody. EC₅₀ values were calculated as described above. Representative data shown are from a single experiment.

To additionally study the ability of a Vγ9×TRBC1 bispecific to mediate γδ T cell cytotoxicity against Jurkat cells with γδ T cells from different donors, γδ T cells were enriched. In particular, Vγ9Vδ2 T cells from 5 different donors (328337, 328676, 327587, 328630, 326287) were expanded from total PBMC population for 13 days. Briefly, PBMC were cultured in the presence of zoledronic acid (Sigma, SML0223) (350 nM, days 0 to 13), rhIL-2 (Miltenyi, 130-097-748) (1000 U/mL days 0-2, 800 U/mL days 2-5, 100 U/mL days 5-13) and rhIL-15 (Miltenyi, 130-095-765) (10 ng/mL days 0-13) in complete growth media (RPMI, 10% HI FBS, 1% Pen/strep). At day 13 of expansion, cells were harvested and enriched with EasySep™ Human Gamma/Delta T Cell Isolation Kit (Stem Cell Technologies, 19255) according to manufacturer's instructions. Following enrichment procedure, cells were seeded at 1×10⁶/mL in complete growth media with addition of 350 nM zoledronic acid, 100 U/mL IL-2 and 10 ng/mL IL-15 and rested overnight.

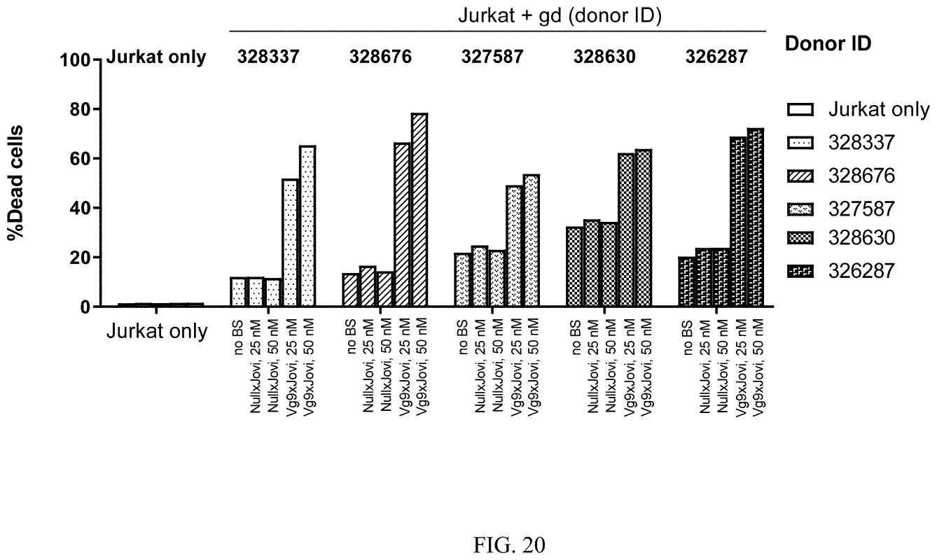

For the killing assay, rested γδ T cells were harvested, and cell number and viability were determined. Target cells (Jurkat cells expressing TRBC1) were labelled with 0.25 μM CFSE (Thermo, C34554) for 5 min. at room temperature. Cells were washed 3 times and cell number and viability were determined. Killing assay at an E:T ratio of 1:1 (10⁵ effector cells: 10⁵ target cells) was set up for 16 hours in 96 well plate in complete growth media in the absence on zoledronic acid and cytokines. Vγ9×Jovi bispecific molecules were adjusted in concentration by limiting dilution to yield final concentration 25 nM and 50 nM in final volume 150 μL/well. After 16 hours, cells were harvested and stained with cocktail containing antibodies against: CD3 (Biolegend, 300424), Vγ9 (Biolegend, 331310), CD25 (Biolegend, 356142), CD69 (Biolegend, 310930), as well as Near-IR (Thermo, L34975) and Fc block (BD, 546219). Cells were washed 3 times, fixed and analysed by flow cytometry. FIG. 20 depicts anti-TRGV9/anti-TRBC1 bispecific antibody mediated cytotoxicity. Vγ9×Jovi bispecific antibodies at both concentrations tested resulted in greater killing in all five of the donor Vγ9Vδ2 T cell populations as compared to the addition of a NULL/Jovi-1 bispecific or no bispecific antibody. The additional of Vγ9×Jovi bispecific antibodies also failed to kill Jurkat cells when donor γδ T cells were not present.

### Example 7—Multispecific Antibodies that Bind TRGV9 and BCMA

Example 7 is based on the premise that γδ T cells, which mainly express heterodimers of TRGV9 and Vδ2 chains demonstrate potent anti-tumor functions. These cells express TCR-TRGV9 and the majority, if not all, of these cells exhibit efficient cytotoxicity of tumor target cells. This ability is then harnessed using bispecific antibodies constructed such that one arm binds to the TRGV9 structure and the other arm binds to a second antigen (BCMA) expressed by the tumor cells (e.g., certain leukemias and lymphomas). Thus, the bispecific antibody bridges the effector and target cells together, resulting in tumor cell killing. This mechanism of action is described in the schematic outlined in FIG. 1.

The subsequent examples can be divided into the following categories: (1) Generation and characterization of bispecific antibodies capable of binding to the TRGV9 arm expressed on γδ T cells and BCMA (Examples 7.1, 7.2, 7.3 and 7.4); and (2) Evidence for bispecific antibody-enabled target cell killing by γδ T cells expanded in vitro (Example 7.5).

### Example 7.1: Anti-VG9 Antibody Generation

Immunogen. A recombinant human TCR Vγ9×Vδ2 fused to a human Fc was used as an immunogen, and the sequence is listed in Table 22.

TABLE 22
Amino acid sequence of recombinant human TCR Vγ9 x Vδ2
heterodimeric protein fused to human Fc

| | Protein | | |
|---|---|---|---|
| Name | ID | Sequence | |
| Recombinant | Vg9 | MAWVWTLLFLMAAAQSIQAAGHLEQPQISSTKTLSKTARL | SEQ ID |
| human [TCR | chain | ECVVSGITISATSVYWYRERPGEVIQFLVSISYDGTVRKE | NO: 156 |
| Vg9 x Vd2]- | | SGIPSGKFEVDRIPETSTSTLTIHNVEKQDIATYYCALWE | |
| hFc | | AQQELGKKIKVFGPGTKLIITDKQLDADVSPKPTIFLPSI | |
| | | AETKLQKAGTYLCLLEKFFPDVIKIHWEEKKSNTILGSQE | |
| | | GNTMKTNDTYMKFSWLTVPEKSLDKEHRCIVRHENNKNGV | |
| | | DQEIIFPPIKTDVITMDPKDNEPKSCDKTHTCPPCPAPEL | |
| | | LGGPSVFLFPPKPKDTLMISRTPEVTCVVVDVSHEDPEVK | |
| | | FNWYVDGVEVHNAKTKPREEQYNSTYRVVSVLTVLHQDWL | |
| | | NGKEYKCKVSNKALPAPIEKTISKAKGQPREPQVYVYPPS | |
| | | REEMTKNQVSLTCLVKGFYPSDIAVEWESNGQPENNYKTT | |
| | | PPVLDSDGSFALVSKLTVDKSRWQQGNVFSCSVMHEALHN | |
| | | HYTQKSLSLSPGK | |
| | Vd2 | MAWVWTLLFLMAAAQSIQAAIELVPEHQTVPVSIGVPATL | SEQ ID |
| | chain | RCSMKGEAIGNYYINWYRKTQGNTMTFIYREKDIYGPGFK | NO: 157 |
| | | DNFQGDIDIAKNLAVLKILAPSERDEGSYYCACDTLGMGG | |
| | | EYTDKLIFGKGTRVTVEPRSQPHTKPSVFVMKNGTNVACL | |
| | | VKEFYPKDIRINLVSSKKITEFDPAIVISPSGKYNAVKLG | |
| | | KYEDSNSVTCSVQHDNKTVHSTDFEVKTDSTDHVKPKETE | |
| | | NTKQPSKSEPKSCDKTHTCPPCPAPELLGGPSVFLFPPKP | |
| | | KDTLMISRTPEVTCVVVDVSHEDPEVKFNWYVDGVEVHNA | |
| | | KTKPREEQYNSTYRVVSVLTVLHQDWLNGKEYKCKVSNKA | |
| | | LPAPIEKTISKAKGQPREPQVYVLPPSREEMTKNQVSLLC | |
| | | LVKGFYPSDIAVEWESNGQPENNYLTWPPVLDSDGSFFLY | |
| | | SKLTVDKSRWQQGNVFSCSVMHEALHNHYTQKSLSLSPGK | |

Protein Production of the Immunogen. Expression plasmids encoding the immunogen (see Table 22) were transfected into CHO cell at a DNA ratio of 1:1. Total amount of DNA for a 750 mL expression scale was 750 μg. Final expression volume was 1 L after two feedings and enhancer additions. Using an ÄKTAPRIME plus instrument (GE Healthcare Life Sciences), supernatant (1 L) after 7 days was applied with a flow-rate of 5 mL/min to a MABSELECT SURE (GE Life Sciences) with a column volume (CV) of 10 mL pre-equilibrated with phosphate buffered saline (PBS), pH 6.8. Non-specific proteins binding to the column material was washed off with PBS supplemented with 500 mM NaCl, pH 6.8 (5 CV). The Fc-containing immunogen was eluted stepwise with 40 mM sodium acetate pH 5.0 (5 CV), pH 4.5 (5 CV), pH 4.0 (10 CV), pH 3.5 (5 CV), and pH 3.0 (5 CV). Fractions were pooled, and applied (5 mL) at a flow-rate of 0.2 mL/min on to a HiLoad 16/600 SUPERDEX (GE Healthcare) column pre-equilibrated with PBS (pH 6.8). Target protein was eluted, pooled, and analyzed by SDS-PAGE, analytic SEC, and intact mass by mass spectrometry. Purity was estimated to >99%.

Antibodies were generated using ALIVAMAB transgenic mice technology (Ablexis). ALIVAMAB mice were immunized with recombinant human Vγ9/Vδ2 TCR protein. Lymphocytes were extracted from secondary lymphoid organs and either fused with FO mouse myeloma cell line for hybridoma generation or subjected to single cell sorting via FACS. Hybridoma supernatants were screened by MSD electrochemiluminescence or by FACS for binding to γδ T cells. Confirmed cell binders were light chain isotyped via ELISA. Single cell sorting supernatants were screened by MSD electrochemiluminescence for binding to recombinant human Vγ9/Vδ2 protein. Several hits with the desired binding profile were selected and sequenced, as provided below.

The CDR sequences of certain VG9 antibodies are provided in Tables 23-26, and the respective VH and VL regions are provided in Tables 27-30, respectively.

TABLE 23
CDR Sequences of anti-TRGV9 antibody
(Vγ9 clone).

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| VG9B420- | GFTFSNYD | 98 | ISSSSSYI | 99 | ARDVGVTD | 100 |
| LH | | | | | YYYYGMDV | |
| | | SEQ | | SEQ | | SEQ |
| | | ID | | ID | | ID |
| Antibody | LCDR1 | NO: | LCDR2 | NO: | LCDR3 | NO: |
| VG9B420- | QSVASSY | 101 | GAS | 102 | QQYGSSP | 103 |
| LH | | | | | PYT | |

TABLE 24
CDR Sequences of anti-TRGV9 antibody
(Vγ9 clone).

| | | SE | | SE | | SE |
|---|---|---|---|---|---|---|
| | | Q | | Q | | Q |
| | | ID | | ID | | ID |
| Antibody | HCDR1 | NO: | HCDR2 | NO: | HCDR3 | NO: |
| VG9SB1OSC1087_ | GDTFNNYA | 107 | IIPFFG | 108 | ARPGSGSPD | 109 |
| P18_D08 | | | TP | | YYYYDMDV | |
| | | SE | | SE | | SE |
| | | Q | | Q | | Q |
| | | ID | | ID | | ID |
| Antibody | LCDR1 | NO: | LCDR2 | NO: | LCDR3 | NO: |
| VG9SB1OSC1087_ | QSLVHSDGN | 110 | KIS | 111 | MQATQFPLT | 112 |
| P18_D08 | TY | | | | | |

TABLE 25
CDR Sequences of anti-TRGV9 antibody (Vγ9 clone).

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| VG9SB1OSC1087_P18_ | GGTFSSYA | 117 | NIPIFN | 118 | VREGTGYS | 119 |
| C12 | | | TA | | YGLDY | |
| | | SEQ | | SEQ | | SEQ |
| | | ID | | ID | | ID |
| Antibody | LCDR1 | NO: | LCDR2 | NO: | LCDR3 | NO: |
| VG9SB1OSC1087_P18_ | QSLIHSDG | 120 | KIS | 121 | MQAKQFP | 122 |
| C12 | NTY | | | | IT | |

TABLE 26
CDR Sequences of anti-TRGV9
antibody (Vγ9 clone).

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| VG9SB1OSC1087_ | GGSISSG | 127 | IYNSGST | 128 | ARDSNYEWF | 129 |
| P19_C03 | GSY | | | | FDL | |
| | | SEQ | | SEQ | | SEQ |
| | | ID | | ID | | ID |
| Antibody | LCDR1 | NO: | LCDR2 | NO: | LCDR3 | NO: |
| VG9SB1OSC1087_ | QSVSSY | 130 | DAS | 131 | QQRSNWPLT | 132 |
| P19_C03 | | | | | | |

TABLE 27
Heavy chain and light chain V-region sequences of anti-TRGV9 antibody
(Vγ9 clone).

| | | SEQ |
|---|---|---|
| | | ID |
| mAb ID | Heavy Chain V-region Amino Acid Sequence | NO: |
| VG9B420-LH | EVQLVESGGGLVKPGGSLRLSCSASGFTFSNYDMNWVRQAPGKGLEWVSSISS | 104 |
| | SSSYTYYADSVKGRFTISRDNAKNSLYLQMNSLRAEDTAVYHCARDVGVTTDY | |
| | YYYGMDVWGQGTMVTVSS | |
| | | SEQ |
| | | ID |
| mAb ID | Light Chain V-region Amino Acid Sequence | NO: |
| VG9B420-LH | EIVMTQSPGTLSLSPGDRATLSCRASQSVASSYLAWYQQKPGQSPRLLIYGAS | 105 |
| | SRATGIPDRFSGSGSGTDFTLTISRLEPEDFAVYYCQQYGSSPPYTFGQGTRL | |
| | EIK | |

TABLE 28
Heavy chain and light chain V-region sequences of anti-TRGV9 antibody
(Vγ9 clone).

| | | SEQ |
|---|---|---|
| | | ID |
| mAb ID | Heavy Chain V-region Amino Acid Sequence | NO: |
| VG9SB10SC1 | EVQLVQSGAEVKKPGSSVKVSCKASGDTFNNYAISWVRQAPGQGLEWMGGIIP | 113 |
| 087_P18_D08 | FFGTPDYAQKFQGRVTITADKSTSTAYMELSGLRSEDTAVYYCARPGSGSPDY | |
| | YYYDMDVWGQGTTVTVSS | |
| | | SEQ |
| | | ID |
| mAb ID | Light Chain V-region Amino Acid Sequence | NO: |
| VG9SB10SC1 | DIVMTQTPLSSPVTLGQPASISCRSSQSLVHSDGNTYLSWLQQRPGQPPRLLI | 114 |
| 087_P18_D08 | YKISNRFSGVPDRFSGSGAGTDFTLKINRVEAEDVGVYYCMQATQFPLTFGGG | |
| | TKVEIK | |

TABLE 29
Heavy chain and light chain V-region sequences of anti-TRGV9 antibody
(Vγ9 clone).

| | | SEQ |
|---|---|---|
| | | ID |
| mAb ID | Heavy Chain V-region Amino Acid Sequence | NO: |
| VG9SB10SC1 | EVQLVQSGAEVKKPGSSVKVSCKASGGTFSSYAISWVRQAPGQGLEWMGGNIP | 123 |
| 087_P18_C12 | IFNTANYAQKFQDRVTITADKSTSTAYMELSSLRSEDTAVYYCVREGTGYSYG | |
| | LDYWGQGTPVTVSS | |
| | | SEQ |
| | | ID |
| mAb ID | Light Chain V-region Amino Acid Sequence | NO: |
| VG9SB10SC1 | EIVMTQSPLSSPVTLGQPASISCRSSQSLIHSDGNTYLSWLQQRPGQPPRLLI | 124 |
| 087_P18_C12 | YKISNRFSGVPDRFSGSGAGTDFTLKISRVEAEDVGIYYCMQAKQFPITFGQG | |
| | TKVDIK | |

TABLE 30
Heavy chain and light chain V-region sequences of anti-TRGV9 antibody
(Vγ9 clone).

| | | SEQ |
|---|---|---|
| | | ID |
| mAb ID | Heavy Chain V-region Amino Acid Sequence | NO: |
| VG9SB10SC1 | QVQLQESGPGLVKPSQTLSLTCTVSGGSISSGGSYWSWIRQHPGKGLEWIGYI | 133 |
| 087_P19_C03 | YNSGSTYYNPSLKSRVSMSVDTSKNQFSLKLSSVTAADTAVYYCARDSNYEWF | |
| | FDLWGPGTLVTVSS | |
| | | SEQ |
| | | ID |
| mAb ID | Light Chain V-region Amino Acid Sequence | NO: |
| VG9SB10SC1 | EIVMTQSPATLSLSPGERATLSCRASQSVSSYLAWYQQKPGQAPRLLIYDASN | 134 |
| 087_P19_C03 | RATGIPARFSGSGSGTDFTLTISSLEPEDFAVYYCQQRSNWPLTFGGGTKVEI | |
| | K | |

Variable Region Cloning. Both RNA purified by QIAGEN kit (RNEASY Plus Mini Kit) and B cells lysate were used for cDNA synthesis using the Smarter cDNA synthesis kit (Clontech, Mount View, Calif.). To facilitate cDNA synthesis, oligodT was used to prime reverse transcription of all messenger RNAs followed by “5′ capping” with a Smarter IIA oligonucleotide. Subsequent amplification of the VH and VL fragments was performed using a two-step PCR amplification using 5′ primers targeting the SMARTER IIA cap and 3′ primers targeting consensus regions in CH1. Briefly, each 50 μl PCR reaction consists of 20 μM of forward and reverse primer mixes, 25 μl of PRIMESTAR MAX DNA polymerase premix (Clontech), 2 μl of unpurified cDNA, and 21 μl of double-distilled H₂O. The cycling program started at 94° C. for 3 min, followed by 35 cycles (94° C. for 30 Sec, 55° C. for 1 min, 68° C. for 1 min), and ended at 72° C. for 7 min. The second round PCR was performed with VL and VH 2nd round primers containing 15 bp complementary extensions that “overlap” respective regions in their respective Lonza mother vector (VH and VL). Second round PCR was performed with the following program: 94° C. for 3 min; 35 cycles (94° C. for 30 Sec, 55° C. for 1 min, 68° C. for 1 min), and ended at 72° C. for 7 min. IN-FUSION® HD Cloning Kit (Clonetech, U.S.A.) was used for directional cloning of VL gene into Lonza huIgK or Lambda vector and VH gene into Lonza huIgG1 vector. To facilitate IN-FUSION® HD Cloning, PCR products were treated with Cloning Enhancer before IN-FUSION HD Cloning. Cloning and transformation were performed according to manufacturer's protocol (Clonetech, U.S.A.). Mini-prep DNAs were subjected to Sanger sequencing to confirm that complete V-gene fragments were obtained.

### Example 7.2: Production and De Novo Sequencing of Anti-TRGV9 Mab

The mouse IgG1 anti-human T cell receptor anti-TRGV9 clone B3 was sourced commercially. Sample preparation and LC-MS/MS analysis were performed by RAPID NOVOR (ON, Canada). Twenty-one in-solution and in-gel digestions were prepared using six different enzymes (Pepsin, Trypsin, Chymotrypsin, Asp N, Lys C, Glu C). The in-solution digestions for the sample was processed with disulfide reduction, alkylation, and then enzyme digestion. Each digestion contains peptides from all immunoglobulin chains. The in-gel digestions were prepared for immunoglobulin chains after gel separation. The sample was processed with disulfide reduction, gel separation, deglycosylation, disulfide reduction a second time, alkylation and then digestion. Digestions were analyzed by LC-MS/MS using THERMO-FISHER Q EXACTIVE™, ORBITRAP FUSION™ mass spectrometers. Peptides were characterized from LC-MS/MS data using de novo peptide sequencing and then assembled into antibody sequences.

The three VH CDR and three VL CDR sequences of anti-human T cell receptor Vγ9 clone B3 are shown in Table 31 (SEQ ID NOs:89-94, respectively); and the VH and VL sequences of the anti-human T cell receptor anti-human T cell receptor Vγ9 clone B3 are shown in Table 32 (SEQ ID NOs:95 and 96, respectively).

TABLE 31
CDR sequences of mouse anti-human TCR
Vγ9 clone B3.

| | | SEQ ID | | SEQ ID | H | SEQ ID |
|---|---|---|---|---|---|---|
| Antibody | HCDR1 | NO: | HCDR2 | NO: | CDR3 | NO: |
| Vg9_B3_RN | GFTFSS | 89 | IHGGTGGI | 90 | ARRGYGA | 91 |
| | NY | | | | WFAY | |
| | | SEQ ID | | SEQ ID | | SEQ ID |
| Antibody | LCDR1 | NO: | LCDR2 | NO: | LCDR3 | NO: |
| Vg9_B3_RN | ENIHNY | 92 | NAK | 93 | QHFWSYP | 94 |
| | | | | | LT | |

TABLE 32
Heavy chain and light chain sequences of mouse anti-human TCR Vγ9
clone B3.

| | Heavy Chain Amino Acid Sequence | SEQ ID |
|---|---|---|
| mAb ID | (from VG9B2) | NO: |
| Vg9_B3_RN | QGQMQQSGAELvKPGASVKLSCKTSGFTFSSNYISWLKQKPGQSLEWIAW | 95 |
| | IHGGTGGIGYNQKFTGKAQLTVDTSSTTAYMQFSSLTTEDSAIYYCARRG | |
| | YGAWFAYWGQGTLVTVSA | |
| | Light Chain Amino Acid Sequence | SEQ ID |
| | (from VG9B2) | NO: |
| Vg9_B3_RN | DIQMTQSPASLSASVGETVTITCRASENIHNYLAWYQQKQGKSPQLLVYN | 96 |
| | AKTLADGVPSRFSGSGSGTQYSLKINSLQPEDFGNYYCQHFWSYPLTFGA | |
| | GTKLELK | |

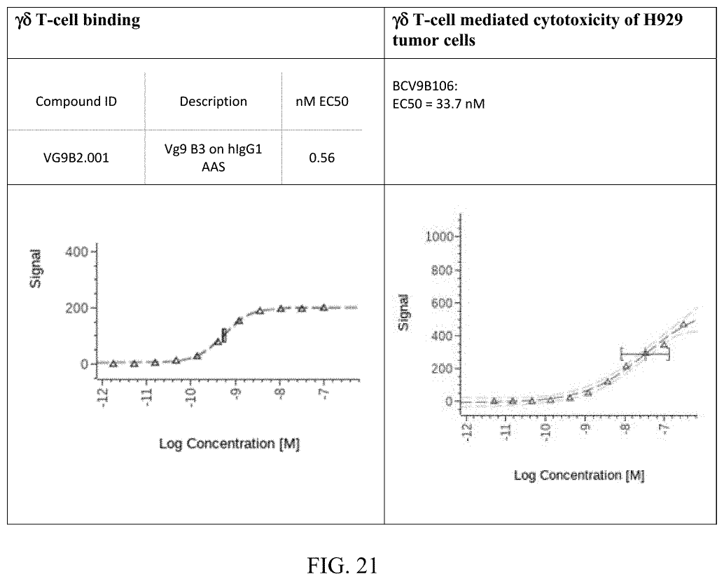

The two antibodies (VG9B2) were expressed in CHO-Expi cells. The purified chimera human IgG1 mAb (silent Fc) demonstrated binding to human γδ T cells showing specificity toward TCR Vγ9, as shown in FIG. 21 (left panel).

### Example 7.3: Production and De Novo Sequencing of Anti-BCMA Mab

An anti-BCMA clone was obtained and sequenced.

TABLE 33
CDR sequences of anti-BCMA mAb.

| | | SEQ | | SEQ | | SEQ |
|---|---|---|---|---|---|---|
| | | ID | | ID | | ID |
| mAb ID | HCDR1 | NO: | HCDR2 | NO: | HCDR3 | NO: |
| BCMB519 | GFTFSSYA | 137 | ISGSGGST | 138 | AKDEGYSSGHYYGMDV | 139 |
| | | SEQ | | SEQ | | SEQ |
| | | ID | | ID | | ID |
| mAb ID | LCDR1 | NO: | LCDR2 | NO: | LCDR3 | NO: |
| BCMB519 | QSISSSF | 140 | GAS | 141 | QHYGSSPMYT | 142 |

TABLE 34
Heavy chain and light chain sequences of anti-BCMA mAb.

| | | SEQ |
|---|---|---|
| | | ID |
| mAb ID | Heavy Chain Amino Acid Sequence | NO: |
| BCMB519 | EVQLLESGGGLVQPGGSLRLSCAASGFTFSSYAMSWVRQAPGKGLE | 143 |
| | WVSAISGSGGSTYYADSVKGRFTISRDNSKNTLYLQMNSLRAEDTAV | |
| | YYCAKDEGYSSGHYYGMDVWGQGTTVTVSS | |
| | | SEQ |
| | | ID |
| mAb ID | Light Chain Amino Acid Sequence | NO: |
| BCMB519 | EIVLTQSPGTLSLSPGERATLSCRASQSISSSFLTWYQQKPGQAPRLLIY | 144 |
| | GASSRATGIPDRFSGGGSGTDFTLTISRLEPEDFAVYYCQHYGSSPMYT | |
| | FGQGTKLEIK | |

### Example 7.4: Preparation of Anti-TRGV9/Anti-BCMA Bispecific Antibodies

The variable region sequence of anti-TRGV9 and anti-BCMA antibodies was used to generate a bispecific human IgG1 antibody to be tested for T cell re-directed killing of H929 cells, which express BCMA. A summary of Vγ9 and BCMA clones is provided in Table 35.

TABLE 35
Summary of Vγ9 and BCMA clones

| | | B # |
|---|---|---|
| i. | VG9B420-LH-scFv Half Ab | BCV9B101 |
| ii. | VG9SB10SC1087_P18_D08-Fab Half Ab | BCV9B100 |
| iii. | VG9SB10SC1087_P18_C12-Fab Half Ab | BCV9B101 |
| iv. | BCMA (BCMB519) | e.g, BCV9B101 |
| v. | VG9SB10SC1087_P19_C03-Fab Half Ab | BCV9B103 |

The bispecific antibodies were produced as Fab (Vg9)×scFv (BCMA) and scFv (Vg9)×Fab (BCMA) antibodies in the knob-into-hole format as human IgG1 with silent Fc. Nucleic acid sequences encoding variable regions were subcloned into a custom mammalian expression vectors containing constant region of human IgG1 expression cassettes using standard PCR restriction enzyme based standard cloning techniques, and sequenced verified. The bispecific antibodies were expressed by transient transfection in Chinese hamster ovary cell line.

The sequences of the bispecific antibodies expressed in the CHO cells are shown in Table 36 below.

TABLE 36
Sequences of antibodies expressed in CHO cells

| | | | SEQ |
|---|---|---|---|
| | | | ID |
| mAb ID | ‘Knob’ arm and ‘hole’ arm amino acid sequence | | NO: |
| BCMA- | Heavy Chain | MAWVWTLLFLMAAAQSIQAEVQLLESGGGLVQPGGSLRLSCAASG | 148 |
| Fab | B | FTFSSYAMSWVRQAPGKGLEWVSAISGSGGSTYYADSVKGRFTIS | |
| (BCMB51 | BCV9B71 | RDNSKNTLYLQMNSLRAEDTAVYYCAKDEGYSSGHYYGMDVWGQG | |
| 9-Fab) | | TTVTVSSASTKGPSVFPLAPSSKSTSGGTAALGCLVKDYFPEPVT | |
| (half-mAb) | | VSWNSGALTSGVHTFPAVLQSSGLYSLSSVVTVPSSSLGTQTYIC | |
| | | NVNHKPSNTKVDKKVEPKSCDKTHTCPPCPAPEAAGGPSVFLFPP | |
| | | KPKDTLMISRTPEVTCVVVSVSHEDPEVKFNWYVDGVEVHNAKTK | |
| | | PREEQYNSTYRVVSVLTVLHQDWLNGKEYKCKVSNKALPAPIEKT | |
| | | ISKAKGQPREPQVYVLPPSREEMTKNQVSLLCLVKGFYPSDIAVE | |
| | | WESNGQPENNYLTWPPVLDSDGSFFLYSKLTVDKSRWQQGNVFSC | |
| | | SVMHEALHNRFTQKSLSLSPGK | |
| | Light Chain | MAWVWTLLFLMAAAQSIQAEIVLTQSPGTLSLSPGERATLSCRAS | 149 |
| | BCV9B71 | QSISSSFLTWYQQKPGQAPRLLIYGASSRATGIPDRFSGGGSGTD | |
| | | FTLTISRLEPEDFAVYYCQHYGSSPMYTFGQGTKLEIKRTVAAPS | |
| | | VFIFPPSDEQLKSGTASVVCLLNNFYPREAKVQWKVDNALQSGNS | |
| | | QESVTEQDSKDSTYSLSSTLTLSKADYEKHKVYACEVTHQGLSSP | |
| | | VTKSFNRGEC | |
| VG9SB10 | Heavy Chain | MAWVWTLLFLMAAAQSIQAEVQLVQSGAEVKKPGSSVKVSCKASG | 150 |
| SC1087_P | A | DTFNNYAISWVRQAPGQGLEWMGGIIPFFGTPDYAQKFQGRVTIT | |
| 18_D08- | BCV9B100 | ADKSTSTAYMELSGLRSEDTAVYYCARPGSGSPDYYYYDMDVWGQ | |
| Fab | | GTTVTVSSASTKGPSVFPLAPSSKSTSGGTAALGCLVKDYFPEPV | |
| (half-mAb) | | TVSWNSGALTSGVHTFPAVLQSSGLYSLSSVVTVPSSSLGTQTYI | |
| | | CNVNHKPSNTKVDKKVEPKSCDKTHTCPPCPAPEAAGGPSVFLFP | |
| | | PKPKDTLMISRTPEVTCVVVSVSHEDPEVKFNWYVDGVEVHNAKT | |
| | | KPREEQYNSTYRVVSVLTVLHQDWLNGKEYKCKVSNKALPAPIEK | |
| | | TISKAKGQPREPQVYVYPPSREEMTKNQVSLTCLVKGFYPSDIAV | |
| | | EWESNGQPENNYKTTPPVLDSDGSFALVSKLTVDKSRWQQGNVFS | |
| | | CSVMHEALHNRFTQKSLSLSPGK | |
| | Light Chain | MAWVWTLLFLMAAAQSIQADIVMTQTPLSSPVTLGQPASISCRSS | 151 |
| | A | QSLVHSDGNTYLSWLQQRPGQPPRLLIYKISNRFSGVPDRFSGSG | |
| | BCV9B100 | AGTDFTLKINRVEAEDVGVYYCMQATQFPLTFGGGTKVEIKRTVA | |
| | | APSVFIFPPSDEQLKSGTASVVCLLNNFYPREAKVQWKVDNALQS | |
| | | GNSQESVTEQDSKDSTYSLSSTLTLSKADYEKHKVYACEVTHQGL | |
| | | SSPVTKSFNRGEC | |
| VG9SB10 | Heavy Chain | MAWVWTLLFLMAAAQSIQAEVQLVQSGAEVKKPGSSVKVSCKASG | 152 |
| SC1087_P | A | GTFSSYAISWVRQAPGQGLEWMGGNIPIFNTANYAQKFQDRVTIT | |
| 18_C12- | BCV9B101 | ADKSTSTAYMELSSLRSEDTAVYYCVREGTGYSYGLDYWGQGTPV | |
| Fab | | TVSSASTKGPSVFPLAPSSKSTSGGTAALGCLVKDYFPEPVTVSW | |
| (half-mAb) | | NSGALTSGVHTFPAVLQSSGLYSLSSVVTVPSSSLGTQTYICNVN | |
| | | HKPSNTKVDKKVEPKSCDKTHTCPPCPAPEAAGGPSVFLFPPKPK | |
| | | DTLMISRTPEVTCVVVSVSHEDPEVKFNWYVDGVEVHNAKTKPRE | |
| | | EQYNSTYRVVSVLTVLHQDWLNGKEYKCKVSNKALPAPIEKTISK | |
| | | AKGQPREPQVYVYPPSREEMTKNQVSLTCLVKGFYPSDIAVEWES | |
| | | NGQPENNYKTTPPVLDSDGSFALVSKLTVDKSRWQQGNVFSCSVM | |
| | | HEALHNRFTQKSLSLSPGK | |
| | Light Chain | MAWVWTLLFLMAAAQSIQAEIVMTQSPLSSPVTLGQPASISCRSS | 153 |
| | A | QSLIHSDGNTYLSWLQQRPGQPPRLLIYKISNRFSGVPDRFSGSG | |
| | BCV9B101 | AGTDFTLKISRVEAEDVGIYYCMQAKQFPITFGQGTKVDIKRTVA | |
| | | APSVFIFPPSDEQLKSGTASVVCLLNNFYPREAKVQWKVDNALQS | |
| | | GNSQESVTEQDSKDSTYSLSSTLTLSKADYEKHKVYACEVTHQGL | |
| | | SSPVTKSFNRGEC | |
| VG9SB10 | Heavy Chain | MAWVWTLLFLMAAAQSIQAQVQLQESGPGLVKPSQTLSLTCTVSG | 154 |
| SC1087_P | A | GSISSGGSYWSWIRQHPGKGLEWIGYIYNSGSTYYNPSLKSRVSM | |
| 19_C03- | BCV9B103 | SVDTSKNQFSLKLSSVTAADTAVYYCARDSNYEWFFDLWGPGTLV | |
| Fab | | TVSSASTKGPSVFPLAPSSKSTSGGTAALGCLVKDYFPEPVTVSW | |
| (half-mAb) | | NSGALTSGVHTFPAVLQSSGLYSLSSVVTVPSSSLGTQTYICNVN | |
| | | HKPSNTKVDKKVEPKSCDKTHTCPPCPAPEAAGGPSVFLFPPKPK | |
| | | DTLMISRTPEVTCVVVSVSHEDPEVKFNWYVDGVEVHNAKTKPRE | |
| | | EQYNSTYRVVSVLTVLHQDWLNGKEYKCKVSNKALPAPIEKTISK | |
| | | AKGQPREPQVYVYPPSREEMTKNQVSLTCLVKGFYPSDIAVEWES | |
| | | NGQPENNYKTTPPVLDSDGSFALVSKLTVDKSRWQQGNVFSCSVM | |
| | | HEALHNRFTQKSLSLSPGK | |
| | Light Chain | MAWVWTLLFLMAAAQSIQAEIVMTQSPATLSLSPGERATLSCRAS | 155 |
| | A | QSVSSYLAWYQQKPGQAPRLLIYDASNRATGIPARFSGSGSGTDF | |
| | BCV9B103 | TLTISSLEPEDFAVYYCQQRSNWPLTFGGGTKVEIKRTVAAPSVF | |
| | | IFPPSDEQLKSGTASVVCLLNNFYPREAKVQWKVDNALQSGNSQE | |
| | | SVTEQDSKDSTYSLSSTLTLSKADYEKHKVYACEVTHQGLSSPVT | |
| | | KSFNRGEC | |

scFv Sequences

| | | | |
|---|---|---|---|
| Vg9-B3- | Heavy Chain | MAWVWTLLFLMAAAQSIQADIQMTQSPASLSASVGETVTITCRAS | 97 |
| LH-scFv | A | ENIHNYLAWYQQKQGKSPQLLVYNAKTLADGVPSRFSGSGSGTQY | |
| (half-mAb) | BCV9B106 | SLKINSLQPEDFGNYYCQHFWSYPLTFGAGTKLELKGGSEGKSSG | |
| | | SGSESKSTGGSQGQMQQSGAELVKPGASVKLSCKTSGFTFSSNYI | |
| | | SWLKQKPGQSLEWIAWIHGGTGGIGYNQKFTGKAQLTVDTSSTTA | |
| | | YMQFSSLTTEDSAIYYCARRGYGAWFAYWGQGTLVTVSAEPKSSD | |
| | | KTHTCPPCPAPEAAGGPSVFLFPPKPKDTLMISRTPEVTCVVVSV | |
| | | SHEDPEVKFNWYVDGVEVHNAKTKPREEQYNSTYRVVSVLTVLHQ | |
| | | DWLNGKEYKCKVSNKALPAPIEKTISKAKGQPREPQVYVYPPSRE | |
| | | EMTKNQVSLTCLVKGFYPSDIAVEWESNGQPENNYKTTPPVLDSD | |
| | | GSFALVSKLTVDKSRWQQGNVFSCSVMHEALHNHYTQKSLSLSPG | |
| | | K | |
| VG9B420- | Heavy Chain | MAWVWTLLFLMAAAQSIQAEIVMTQSPGTLSLSPGDRATLSCRAS | 106 |
| LH-scFv | A | QSVASSYLAWYQQKPGQSPRLLIYGASSRATGIPDRFSGSGSGTD | |
| (half-mAb) | BCV9B71 | FTLTISRLEPEDFAVYYCQQYGSSPPYTFGQGTRLEIKGGSEGKS | |
| | | SGSGSESKSTGGSEVQLVESGGGLVKPGGSLRLSCSASGFTFSNY | |
| | | DMNWVRQAPGKGLEWVSSISSSSSYIYYADSVKGRFTISRDNAKN | |
| | | SLYLQMNSLRAEDTAVYHCARDVGVTTDYYYYGMDVWGQGTMVTV | |
| | | SSEPKSSDKTHTCPPCPAPEAAGGPSVFLFPPKPKDTLMISRTPE | |
| | | VTCVVVSVSHEDPEVKFNWYVDGVEVHNAKTKPREEQYNSTYRVV | |
| | | SVLTVLHQDWLNGKEYKCKVSNKALPAPIEKTISKAKGQPREPQV | |
| | | YVYPPSREEMTKNQVSLTCLVKGFYPSDIAVEWESNGQPENNYKT | |
| | | TPPVLDSDGSFALVSKLTVDKSRWQQGNVFSCSVMHEALHNHYTQ | |
| | | KSLSLSPGK | |
| BCMA- | Heavy Chain | MAWVWTLLFLMAAAQSIQAEIVLTQSPGTLSLSPGERATLSCRAS | 145 |
| scFv | B | QSISSSFLTWYQQKPGQAPRLLIYGASSRATGIPDRFSGGGSGTD | |
| (BCMB51 | BCV9B101 | FTLTISRLEPEDFAVYYCQHYGSSPMYTFGQGTKLEIKGGSEGKS | |
| 9-scFv) | | SGSGSESKSTGGSEVQLLESGGGLVQPGGSLRLSCAASGFTFSSY | |
| (half-mAb) | | AMSWVRQAPGKGLEWVSAISGSGGSTYYADSVKGRFTISRDNSKN | |
| | | TLYLQMNSLRAEDTAVYYCAKDEGYSSGHYYGMDVWGQGTTVTVS | |
| | | SEPKSSDKTHTCPPCPAPEAAGGPSVFLFPPKPKDTLMISRTPEV | |
| | | TCVVVSVSHEDPEVKFNWYVDGVEVHNAKTKPREEQYNSTYRVVS | |
| | | VLTVLHQDWLNGKEYKCKVSNKALPAPIEKTISKAKGQPREPQVY | |
| | | VLPPSREEMTKNQVSLLCLVKGFYPSDIAVEWESNGQPENNYLTW | |
| | | PPVLDSDGSFFLYSKLTVDKSRWQQGNVFSCSVMHEALHNHYTQK | |
| | | SLSLSPGK | |

TABLE 37
Anti-TRGV9 and Anti-BCMA Heavy and Light Chain Sequences

| Antibody | Heavy Chain | Light Chain |
|---|---|---|
| VG9SB10 | EVQLVQSGAEVKKPGSSVKVSCKASGDTF | DIVMTQTPLSSPVTLGQPASISCRSSQSL |
| SC1087_ | NNYAISWVRQAPGQGLEWMGGIIPFFGTP | VHSDGNTYLSWLQQRPGQPPRLLIYKISN |
| P18_D08 | DYAQKFQGRVTITADKSTSTAYMELSGLR | RFSGVPDRFSGSGAGTDFTLKINRVEAED |
| | SEDTAVYYCARPGSGSPDYYYYDMDVWGQ | VGVYYCMQATQFPLTFGGGTKVEIKRTVA |
| | GTTVTVSSASTKGPSVFPLAPSSKSTSGG | APSVFIFPPSDEQLKSGTASVVCLLNNFY |
| | TAALGCLVKDYFPEPVTVSWNSGALTSGV | PREAKVQWKVDNALQSGNSQESVTEQDSK |
| | HTFPAVLQSSGLYSLSSVVTVPSSSLGTQ | DSTYSLSSTLTLSKADYEKHKVYACEVTH |
| | TYICNVNHKPSNTKVDKKVEPKSCDKTHT | QGLSSPVTKSFNRGEC |
| | CPPCPAPEAAGGPSVFLFPPKPKDTLMIS | (SEQ ID NO: 116) |
| | RTPEVTCVVVSVSHEDPEVKFNWYVDGVE | |
| | VHNAKTKPREEQYNSTYRVVSVLTVLHQD | |
| | WLNGKEYKCKVSNKALPAPIEKTISKAKG | |
| | QPREPQVYVYPPSREEMTKNQVSLTCLVK | |
| | GFYPSDIAVEWESNGQPENNYKTTPPVLD | |
| | SDGSFALVSKLTVDKSRWQQGNVFSCSVM | |
| | HEALHNRFTQKSLSLSPGK | |
| | (SEQ ID NO: 115) | |
| VG9SB1 | EVQLVQSGAEVKKPGSSVKVSCKASGGTF | EIVMTQSPLSSPVTLGQPASISCRSSQSL |
| 0SC108 | SSYAISWVRQAPGQGLEWMGGNIPIFNTA | IHSDGNTYLSWLQQRPGQPPRLLIYKISN |
| 7_P18_ | NYAQKFQDRVTITADKSTSTAYMELSSLR | RFSGVPDRFSGSGAGTDFTLKISRVEAED |
| C12 | SEDTAVYYCVREGTGYSYGLDYWGQGTPV | VGIYYCMQAKQFPITFGQGTKVDIKRTVA |
| | TVSSASTKGPSVTSGGTAALGCLVKDYFP | APSVFIFPPSDEQLKSGTASVVCLLNNFY |
| | EPVTVSWNSGALTSGVHTFPAVLQSSGLY | PREAKVQWKVDNALQSGNSQESVTEQDSK |
| | SLSSVVTVPSSSLGTQTYICNVNHKPSNT | DSTYSLSSTLTLSKADYEKHKVYACEVTH |
| | KVDKKVEPKSCDKTHTCPPCPAPEAAGGP | QGLSSPVTKSFNRGEC |
| | SVFLFPPKPKDTLMISRTPEVTCVVVSVS | (SEQ ID NO: 126) |
| | HEDPEVKFNWYVDGVEVHNAKTKPREEQY | |
| | NSTYRVVSVLTVLHQDWLNGKEYKCKVSN | |
| | KALPAPIEKTISKAKGQPREPQVYVYPPS | |
| | REEMTKNQVSLTCLVKGFYPSDIAVEWES | |
| | NGQPENNYKTTPPVLDSDGSFALVSKLTV | |
| | DKSRWQQGNVFSCSVMHEALHNRFTQKSL | |
| | SLSPGK | |
| | (SEQ ID NO: 125) | |
| VG9SB1 | QVQLQESGPGLVKPSQTLSLTCTVSGGSI | EIVMTQSPATLSLSPGERATLSCRASQSV |
| 05C108 | SSGGSYWSWIRQHPGKGLEWIGYIYNSGS | SSYLAWYQQKPGQAPRLLIYDASNRATGI |
| 7_P19_ | TYYNPSLKSRVSMSVDTSKNQFSLKLSSV | PARFSGSGSGTDFTLTISSLEPEDFAVYY |
| C03 | TAADTAVYYCARDSNYEWFFDLWGPGTLV | CQQRSNWPLTFGGGTKVEIKRTVAAPSVF |
| | TVSSASTKGPSVFPLAPSSKSTSGGTAAL | IFPPSDEQLKSGTASVVCLLNNFYPREAK |
| | GCLVKDYFPEPVTVSWNSGALTSGVHTFP | VQWKVDNALQSGNSQESVTEQDSKDSTYS |
| | AVLQSSGLYSLSSVVTVPSSSLGTQTYIC | LSSTLTLSKADYEKHKVYACEVTHQGLSS |
| | NVNHKPSNTKVDKKVEPKSCDKTHTCPPC | PVTKSFNRGEC |
| | PAPEAAGGPSVFLFPPKPKDTLMISRTPE | (SEQ ID NO: 136) |
| | VTCVVVSVSHEDPEVKFNWYVDGVEVHNA | |
| | KTKPREEQYNSTYRVVSVLTVLHQDWLNG | |
| | KEYKCKVSNKALPAPIEKTISKAKGQPRE | |
| | PQVYVYPPSREEMTKNQVSLTCLVKGFYP | |
| | SDIAVEWESNGQPENNYKTTPPVLDSDGS | |
| | FALVSKLTVDKSRWQQGNVFSCSVMHEAL | |
| | HNRFTQKSLSLSPGK | |
| | (SEQ ID NO: 135) | |
| BCMA | EVQLLESGGGLVQPGGSLRLSCAASGFTF | EIVLTQSPGTLSLSPGERATLSCRASQSI |
| (BCMB5 | SSYAMSWVRQAPGKGLEWVSAISGSGGST | SSSFLTWYQQKPGQAPRLLIYGASSRATG |
| 19) | YYADSVKGRFTISRDNSKNTLYLQMNSLR | IPDRFSGGGSGTDFTLTISRLEPEDFAVY |
| | AEDTAVYYCAKDEGYSSGHYYGMDVWGQG | YCQHYGSSPMYTFGQGTKLEIKRTVAAPS |
| | TTVTVSSASTKGPSVFPLAPSSKSTSGGT | VFIFPPSDEQLKSGTASVVCLLNNFYPRE |
| | AALGCLVKDYFPEPVTVSWNSGALTSGVH | AKVQWKVDNALQSGNSQESVTEQDSKDST |
| | TFPAVLQSSGLYSLSSVVTVPSSSLGTQT | YSLSSTLTLSKADYEKHKVYACEVTHQGL |
| | YICNVNHKPSNTKVDKKVEPKSCDKTHTC | SSPVTKSFNRGEC |
| | PPCPAPEAAGGPSVFLFPPKPKDTLMISR | (SEQ ID NO: 147) |
| | TPEVTCVVVSVSHEDPEVKFNWYVDGVEV | |
| | HNAKTKPREEQYNSTYRVVSVLTVLHQDW | |
| | LNGKEYKCKVSNKALPAPIEKTISKAKGQ | |
| | PREPQVYVLPPSREEMTKNQVSLLCLVKG | |
| | FYPSDIAVEWESNGQPENNYLTWPPVLDS | |
| | DGSFFLYSKLTVDKSRWQQGNVFSCSVMH | |
| | EALHNRFTQKSLSLSPGK | |
| | (SEQ ID NO: 146) | |

The antibodies were initially purified by MAB SELECT SURE PROTEIN A column (GE Healthcare). The column was equilibrated with PBS pH 7.2 and loaded with fermentation supernatant at a flow rate of 2 mL/min. After loading, the column was washed with 4 column volumes of PBS followed by elution in 30 mM sodium acetate, pH 3.5. Fractions containing protein peaks as monitored by absorbance at 280 nm were pooled and neutralized to pH 5.0 by adding 1% 3 M sodium acetate pH 9.0. The bispecific mAbs were further purified on a preparative SUPERDEX 200 10/300 GL (GE healthcare) size exclusion chromatography (SEC) column equilibrated with PBS buffer. The integrity of sample was assessed by endotoxin measurement (<3.0 EU/mg), SDS-PAGE under reducing and non-reducing conditions, SEC, and intact mass by MS.

### Example 7.5: Evaluation of Binding and Cytotoxic Properties of the Anti-TRGV9/Anti-BCMA Bispecific Antibody Using H929 Cells and Human γδ T Cells

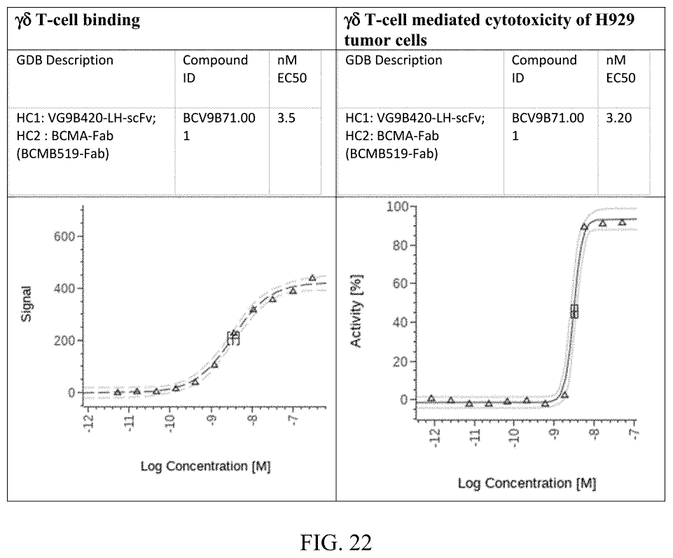

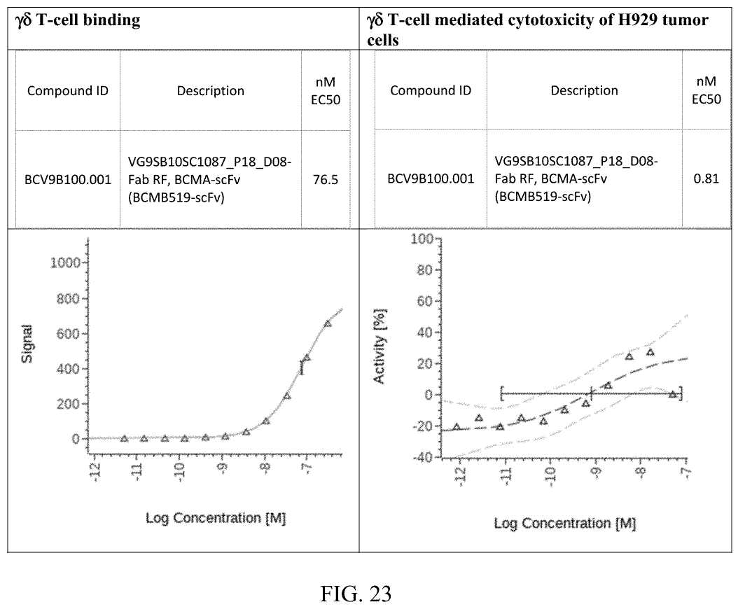

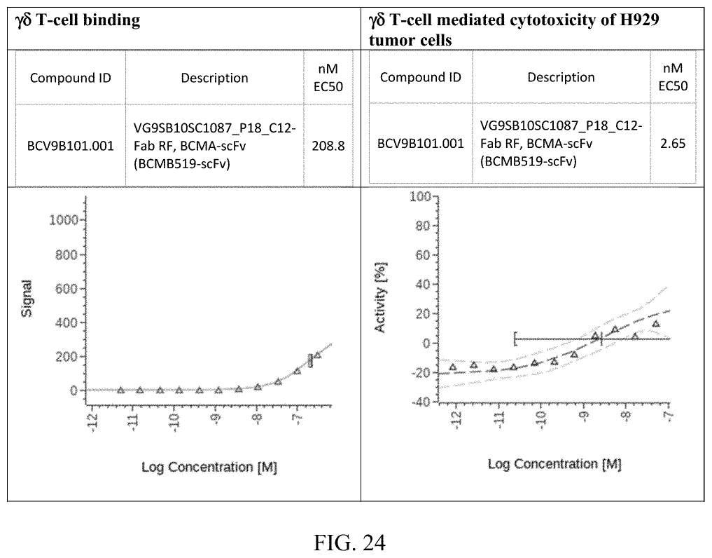

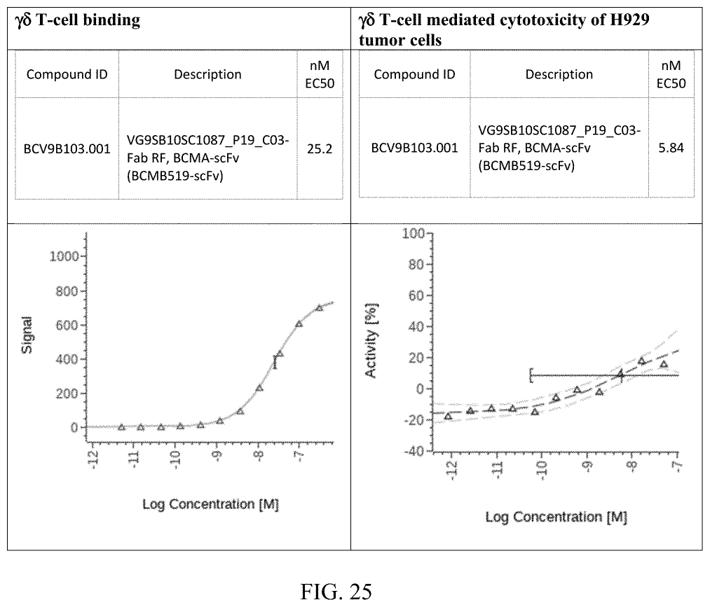

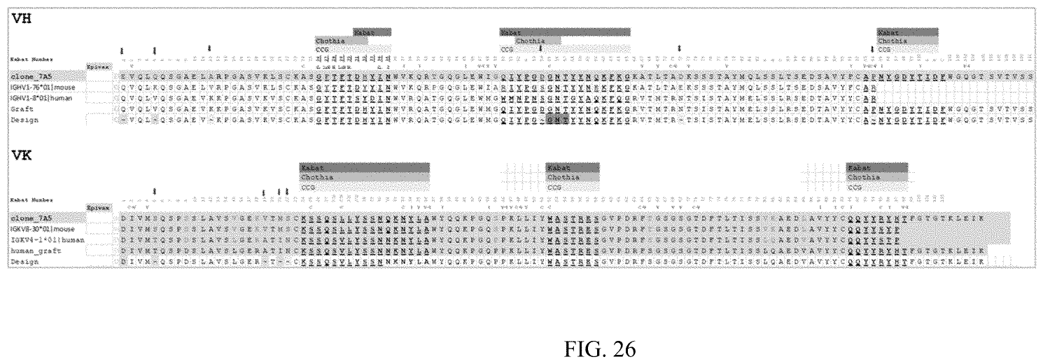

Each of FIGS. 21-25 shows that the anti-TRGV9/anti-BCMA bispecific antibodies bind γδ T cells (left panels) and mediate γδ T cell cytotoxicity against BCMA expressing H929 cells in vitro (right panels). For the binding assays, γδ-enriched T cells were used, and samples incubated for 1 hour at 37° C. prior to measurements. For the killing assays, expanded γδ T cells (effectors) were co-cultured with H929 at 5:1 E:T ratios in the presence of various concentrations of the bispecific antibody for 72 hours at 37° C. Bispecific constructs were tested in 11-point titration curve with a 3-fold dilution series starting at 50 nM antibody concentration. Human pan T cells were used as effector cells, as was done previously (see above). H929-WT tumor cell line was used as target cells. Dose response curves show anti-TRGV9/anti-BCMA bispecific mediated T cell cytotoxicity against BCMA expressing H929 cells in a dose dependent manner. EC₅₀ values were calculated as described in methods. Representative data shown are from a single experiment.

### Example 8—Multispecific Antibodies that Bind Trgv9 and CD123

Examples 8.1-8.10 are based on the premise that γδ T cells, which mainly express heterodimers of TRGV9 and Vδ2 chains demonstrate potent anti-tumor functions. These cells express TCR-TRGV9 and the majority, if not all, of these cells exhibit efficient cytotoxicity of tumor target cells. This ability is then harnessed using bispecific antibodies constructed such that one arm binds to the TRGV9 structure and the other arm binds to a tumor-associated antigen expressed by the tumor cells. Thus, the bispecific antibody bridges the effector and target cells together-resulting in tumor cell killing. This mechanism of action is described in the schematic outlined in FIG. 1.

### Example 8.1: Materials and Methods

Cell lines and reagents. Cell lines (Kasumi-3; acute myeloblastic leukemia cell line and 22Rv1; prostate epithelial cell line) used in this study were purchased from ATCC. Kasumi-3 and 22Rv1 cells were cultured in RPMI-1640+20% FBS+lx Pen/Strep and RPMI-140+10% FBS+lx Pen/Strep media respectively. Five to ten vials of cells from initial passages were frozen as stocks, from which cells with fewer than 20 passages were used for all the experiments for both cell lines. All cell culture media and supplements were purchased from Gibco (Thermo Fisher Scientific Inc, Waltham, Mass.). CFSE (Carboxyfluorescein succinimidyl ester) and 7-AAD (7-Aminoactinomycin D) reagents were obtained from ThermoFisher scientific and BioLegend respectively.

PBMC Isolation. Peripheral Blood Mononuclear Cells (PBMCs) were isolated from healthy donor's blood as described elsewhere. Briefly, whole blood was diluted in plain RPMI-1640 medium at 1:1 ratio and carefully layered onto Lymphoprep™ gradient (STEMCELL Technologies, Vancouver, Canada) in a 50 mL falcon Tube (Corning, N.Y., USA). Centrifuged the tube at 450×g for 30 min at room temperature with acceleration and deacceleration was kept at 0. After centrifugation, cells were collected from the interface and erythrocyte lysis was performed using RBC lysis buffer (Sigma, St. Louis, Mo.) for 5 min at room temperature. Supernatant containing lysed erythrocyte was discarded and the cell pellet was washed twice with plain RPMI-1640 medium. After washes, cells were resuspended in culture medium (RPMI-1640+10% FBS+1×Pen/Strep), counted and used it for downstream applications or frozen down in the freezing medium (90% FBS+10% DMSO) at a density of 25×10⁶ cells/mL and stored in liquid nitrogen until further use.

Isolation and expansion of Vγ9⁺ (γδ) T cells from whole PBMCs. Selective expansion of Vγ9⁺ (γδ) T cells from whole PBMCs was achieved using Zoledronic acid. PBMCs were isolated from healthy individual or cancer patient's blood, as described in earlier section. For selective expansion of Vγ9⁺γδ T cells, isolated PBMCs were cultured in culture medium (RPMI-1640 containing 10% FBS, 1× Pen/Strep) supplemented with recombinant human IL-2 (rhIL-2) (to a final concentration of 1000, 400 and 100 IU/mL on day 0, 2 and rest of the 14 day culture period respectively), recombinant human IL-15 (rhIL-15) to a final concentration of 10 ng/mL (on all days of the culture period) and Zoledronic acid to a final concentration of 5 μM (on day 0) for 14 days. Culture medium containing IL-2 and 11-15 was replenished once in 2-3 days and the cultures were transferred into new flasks as necessitated by the growth of cells. γδ T cells were negatively enriched from day 14 PBMCs stimulated with Zol+IL-2+IL-15 (or from day 0 PBMCs) using EasySep™ Human γδ T cell isolation kit, as per the manufacturer's instructions. Enriched γδ T cells were verified for their purity by staining the cells, before and after enrichment, with antibodies against TCRγδ, TCRVγ9 and αβ T cells.

Flow cytometry. All flow cytometry studies were carried out on Novocyte flow cytometer (ACEA biosciences, Singapore) and data was analyzed by FlowJo analysis software (Treestar Inc, Ashland, Oreg.). All antibodies used in this study were purchased from commercial suppliers (BioLegend), unless and until specified. For surface staining, 0.1-0.2 million cells were initially Fc blocked with human TruStain FcX™ (Biolegend, San Diego, Calif.) in the culture medium (RPMI-1640+10% FBS+1×Pen/Str) for 20 minutes at 4° C. Washed twice with wash buffer (PBS+2% FBS) and stained with LIVE/DEAD™ Fixable violet dye (Thermo Fischer Scientific Inc, Waltham, Calif.) in PBS for 20 minutes at room temperature. Alternatively, cells were incubated in PBS containing human TruStain FcX™ and LIVE/DEAD™ Fixable violet dye at 4° C. for 30 minutes. For surface staining, cells were stained with antibodies specific for cell surface antigens for 30 minutes at 4° C. After incubation period, cells were washed twice with wash buffer and acquired on Novocyte flow cytometer immediately. Alternatively, cells were fixed with BD Cytofix (BD Biosciences) for 30 minutes at 4° C., washed twice with wash buffer, resuspended in wash buffer (PBS+2% FBS) and acquired on flow cytometer within 48 hours of fixation. For intracellular staining, surface stained cells were fixed and permeabilized by resuspending them in 100 μL BD Cytofix/Cytoperm (BD Biosciences), and incubated the cell suspension at 4° C. for 15-30 min in dark. After two washes with 200 μL of 1× Perm/Wash buffer, cells were probed by incubating them in 100 μL of Perm/Wash buffer containing antibodies against intracellular effector molecules (Granzyme B and Perforin) at 4° C. for 30 min in dark. After incubation period, cells were washed twice and resuspended in wash buffer (PBS+2% FBS) and acquired on Flow cytometer.

Depletion of immune cell subsets from whole PBMCs. Depletion of specific cell subsets was carried out by FACS-sorting. For flow cytometry based cell sorting, Pan-T cells were enriched using EasySep™ human T cell isolation kit (Stem cell Technologies), as per the manufacturer's instructions. Enriched cells were washed twice with wash buffer (PBS+2% FBS) and stained with anti-human Vγ9 antibody for 30 minutes at 4° C. Stained cells were FACS sorted as Vγ9⁻ Pan-T cells on FACS ARIA (BD biosciences, San Jose, Calif.). Sorted Vγ9⁻ T cells purity was assessed on flow cytometry.

Bispecific antibody binding assay. Binding of anti-TRGV9×CD123 (VG1) and anti-TRGV9×Null (VG3) bispecific antibodies to CD123 expressing cell line (Kasumi-3) and γδ T cells was carried out by flow cytometry. Briefly, 50,000 target cells (Kasumi-3) or γδ T cells were initially incubated in PBS containing human TruStain FcX™ Fc block and LIVE/DEAD™ Fixable violet dye at 4° C. for 30 minutes. After washing, cells were incubated in wash buffer (PBS+2% FBS) containing serial diluted bispecific or it's respective null arm control antibody at 4° C. for 45 minutes. After washing with wash buffer (PBS+2% FBS), cell surface bound bispecific antibody was detected by incubating the cells with PE labelled mouse anti human IgG4 secondary antibody (SouthernBiotech, Birmingham, Ala.) for 30 minutes at 4° C. After the incubation period, cells were washed with wash buffer (PBS+2% FBS) and the fluorescence of stained cells was measured on Novocyte flow cytometer. Cells were visualized on forward and sideward scatter and doublets were excluded. No secondary antibody control was used to establish background fluorescence and to gate on specific population. Background value was subtracted from main samples to get specific binding value.

Flow cytometry-based cell-cell conjugate formation assay. Cell to cell conjugate formation assay was carried out as described in the literature. Briefly, γδ T cells were enriched from fresh PBMCs as described in earlier sections. Enriched effector cells (γδ T cells) and target cells (Kasumi-3) were labelled with 0.3 μM CellTracker™ Green CMFDA (Life Technologies, Carlsbad, Calif.) and 1.5 μM CellTracker™ Orange CMRA (Life Technologies, Carlsbad, Calif.) dyes respectively. Labelled cells were resuspended in culture medium at a concentration of 1.0×10⁶ cells/mL. One hundred microliters of effector and target cells of each were co-cultured in the presence or absence of specified bispecifics at a concentration of 1 μg/mL in a U-bottom 96-well cell culture plate. Cells were incubated at 37° C., 5% CO₂ for one hour in a humidified incubator. After incubation period, cells were spun down and washed once with wash buffer and acquired on Novocyte flow cytometer. For fixing the cell-cell conjugates, after washes, cells were fixed by incubating them with BD Cytofix (BD Biosciences) for 15 minutes at 4° C. Washed the cells and acquired on Novocyte flow cytometer. For analyzing cell-cell conjugate formation, doublets were included while analyzing the data on FlowJo analysis software (Treestar Inc, Ashland, Oreg.).

In vitro cytotoxicity assay. Efficacy of VG1 (anti-TRGV9×CD123) and VG3 (V anti-TRGV9×Null) bispecific antibody in mediating tumor cell lysis was assessed by flow cytometry-based cytotoxicity assay. In brief, target cells were labelled with CFSE by incubating the cells in 0.5 μM CFSE (Thermo Fischer Scientific Inc., Waltham, Calif.) at room temperature for 8 min. After staining period, labelling was stopped by adding 1 mL of FBS (Gibco) and 8 mL of culture medium (RPMI+10% FBS, 1×Pen/Str). After washing twice with culture medium, cells were counted and resuspended in complete medium. Enriched γδ T cells (Effectors), isolated from PBMCs cultured with Zoledronic acid+IL-2+IL-15 for 12 days, were co-cultured with CFSE labelled Kasumi-3 cells (Targets) at different E:T ratios in the presence of various concentrations of the bispecific antibody for 24 hours. At the end of the incubation period, 7-AAD (BioLegend) was added to the effector-target co-culture and acquired cells on Novocyte flow cytometer. To identify target cell death, CFSE positive cells were initially gated on to identify the target cells. Within the CFSE positive target cells, dead cells were identified as 7-AAD⁺ FSC^{low} cells. Gates were set based on the CFSE unstained and 7-AAD unstained cells. To calculate bispecific antibody mediated specific killing, cell lysis value from no bispecific antibody control well was subtracted from total cell death value from the wells containing indicated bispecific antibodies. Spontaneous cytotoxicity of target cells was assessed by culturing them without effector cells or bispecific antibodies. EC₅₀ was calculated using a 4-parameter dose-response curve with the concentration on the x-axis (log scale) and specific lysis on the y-axis (linear scale) using GraphPad Prism version 8 (La Jolla, Calif., USA).

Whole PBMCs assay. Whole PBMC assay was carried out to assess the efficacy of bispecific antibody in mediating activation, proliferation, differentiation and effector profile of γδ T cells. Briefly, CFSE labelled whole PBMCs (0.1×10⁶ cells in 200 of culture medium) were spiked in with Kasumi-3 cells (at a ratio of 1:1 Vγ9⁺ (γδ) T cells and Kasumi-3 cells) and cultured in the absence or presence of indicated bispecific antibodies at a concentration of 3 ng/mL as described above. Activation and proliferation of Vγ9⁺ (γδ) T cells among whole PBMCs was assessed by measuring the surface expression of CD69, CD25 and CFSE dilution respectively. For assessing bispecific mediated Vγ9⁺γδ T cells cytotoxicity, whole\ PBMCs were spiked in with CFSE labeled Kasumi-3 cells, as mentioned earlier. Elimination of Kasumi-3 cells (% 7-AAD⁺ cells among CFSE⁺ Kasumi-3 cells), as a measure of cytotoxicity, was measured on day 5 of the culture. Bispecific mediated Vγ9⁺γδ T cells specific cytotoxicity was calculated by deducting the basal cytotoxicity, as described in above sections.

Cytokine and effector molecule Analysis. Cytokines and effector molecules were assessed both intracellularly and in the cell culture supernatants. For intracellular cytokine and effector molecules assessment by flow cytometry, cells were initially surface stained with indicated monoclonal antibodies and washed twice with wash buffer. Stained cells were fixed and permeabilized using BD Fix/Perm kit (BD biosciences) as per manufacturer's instructions. Permeabilized cells were probed with monoclonal antibodies against intracellular cytokines (TNFα, IFNγ) or effector molecules (Granzyme B, Perforin) for 30 minutes at 4° C. Cells were washed twice and acquired on Novocyte flow cytometer. FMO (Fluorescence Minus One) controls were used to establish the gating for cytokines. For assessing cytokines in cell culture supernatant, cell culture supernatants were collected on indicated time points and were subjected to quantification using customized human magnetic Luminex assay 15 plex kit (R& D systems, Minneapolis, USA), as per the manufacturer's instructions. Quantification of the cytokines was carried out in MagPix multiplex detection system with xPONENT software.

Xenograft Tumor model. This in vivo study was performed following the procedure described by Daniela Wesch with slight modification. All animal experiments were performed in strict accordance with rules approved by an in-house animal committee (IAEC SYNGENE/IAEC/1140/02-2020). Five to six weeks old NOD/MrkBomTac-Prkdc^{scid} (NOD SCID-F) mice were purchased from Vivo Biotech Ltd. The animals were quarantined for 1 week, followed by 1 week for acclimatization at in-house animal facility before starting of the experiment. All mice received a single s.c. injection of 5×10⁶ KG-1 cells, mixed with 1:1 ratio of Matrigel, in the right flank. Two days post s.c injection, mice were randomized based on body weight and segregated into four groups of 6 mice each. After the randomization (Day 2), subcutaneous injection of either 15 μg/kg IL-2 with PBS or 1.5 mg/kg (VG7A5-29×CD123) was carried out. Treatments were repeated weekly for a total of 4 weeks. Where indicated, mice received previously expanded Vγ9+ γδ T cells of one donor subcutaneously on day 2 (2.5×10⁶/mouse), day 7 (8×10⁶/mouse), day 14 (4.5×10⁶/mouse), and day 23 (6×10⁶/mouse). The total volume of IL-2, plus PBS or (VG7A5-29×CD123) and PBS or Vγ9+ γδ T cells was 200 μL. Body weight and tumor volume were measured once every three days. Tumor volume (TV) was determined by measurements in two dimensions using digital Vernier calipers. Tumor volume (TV) was calculated using the following formula: Tumor Volume (mm³)=L×W/2. L=length (mm), W=width (mm). Mice were euthanized if the mean tumor volume is >1000 mm³ or the drop-in body weight loss is >20% (whichever is earlier).

Statistical analysis. For the evaluation of the statistical significance of tumor growth inhibition & body weight changes, two-way ANOVA followed by Bonferroni's post-test was performed using Graph Pad Prism (Version 8.3.0). p values≤0.05 were considered as a statistically significant difference between groups.

Production of recombinant antigen (Vγ9-Vδ2-Fc) protein. De novo sequencing of anti-Vγ9 clone 7A5 mAb was performed as previously described in Example 2.1. The Vγ9×CD123 bispecific antibody was prepared as previously described in Example 2.2.

The extracellular domain of heterodimer Vγ9-Vδ2 TCR containing a C-terminal human IgG1 Fc tag was expressed as a secreted protein in ExpiCHO cell line. Purification protocol identical to the mAbs, except that the final protein was dialyzed into PBS pH 6.8. Purity was determined by SDS-PAGE and SEC to 99.5%.

HDX epitope mapping of 7A5 mAb on TCR Vγ9 protein. The procedures used to analyze the mAb perturbation were carried out as previously described with minor modifications. Huang et al., Mabs. 2018. 10:95-103; Horn et al., Biochemistry. 2006 45:8488-8498; Hamuro et al., J. Biomol. Tech. 2003. 14:171-182. Recombinant human Vγ9-Vδ2 was incubated with and without anti-Vγ9 7A5 mAb (in BSA-free PBS pH 7.2 buffer) in 118 μL deuterium oxide labeling buffer (50 mM sodium phosphate, 100 mM sodium chloride at pD 7.4) at 10° C. At time points 0 sec, 10 sec, 60 sec, 300 sec, 1800 sec or 7200 sec, hydrogen-deuterium exchange (HDX) mixture was quenched by adding 130 μL of 4 M guanidine hydrochloride, 0.85 M TCEP buffer followed by a 3 min incubation at 10° C. Final pH is ˜2.5. The quenched samples were subjected to online pepsin/protease XIII digestion using an in-house packed pepsin/protease XIII column (2.1×30 mm). The resultant peptides were analyzed using an UPLC-MS system comprised of a Waters Acquity UPLC coupled to a Q Exactive™ Hybrid Quadrupole-Orbitrap Mass Spectrometer (Thermo). The peptides were separated on a 50×1 mm C8 column with a 16.5 min gradient from 2-31% solvent B (0.2% formic acid in acetonitrile). Solvent A was 0.2% formic acid in water. The injection valve and pepsin/protease XIII column and their related connecting tubing's were inside a cooling box maintained at 15° C. for native protein. The second switching valve, C8 column and their related connecting stainless-steel tubing's were inside another chilled circulating box maintained at −6° C. Peptide identification was done through searching MS/MS data against the Vγ9-Vδ2 TCR sequence with Mascot. The mass tolerance for the precursor and product ions were 7 ppm and 0.02 Da, respectively. Raw MS data was processed using HDX WorkBench, software for the analysis of HDX MS data Pascal et al., J. Am. Soc. Mass. Spectrom. 2012. 23:1512-1521. The deuterium levels were calculated using the average mass difference between the deuterated peptide and its native form (t0).

Binding kinetics for anti-Vγ9 mAb to TCR Vγ9-Vδ2_Fc by SPR. Data was obtained using ProteOn XPR36 Surface Plasmon Resonance (SPR) System from BioRad. The experiments were carried out in HB SP buffer at xx ° C. The experimental set up was following: Goat anti-murine Fc surface was immobilized on a GLC chip, and binding was tested by capturing the mouse anti-human TCR Vγ9 [clone 7A5] mAb at different densities. The monovalent Vγ9-Vδ2 heterodimer fused to human Fc construct flew in to bind in solution at 0.3 μM in 3-fold dilution series. Association and dissociation times was set to 4 min and 30 min, respectively. Raw binding data were processed by double referencing: 1) interspot on an empty chip surface; 2) column 6 where no 7A5 was captured, to monitor the noise to background of the antigen binding to the GAM-Fc capture surface. Data was global fitted to a 1:1 simple Langmuir binding model.

### Example 8.2 Vγ9 Mab Bind to TCR Vγ9 CDR3 Region

Anti-TRGV9 bispecific antibody was humanized as previously described in Example 5. To map the binding site on recombinant human Vγ9-Vδ2 TCR, epitope mapping was performed by Hydrogen exchange by mass spectrometry (HXMS) technology. Clone 7A5 was incubated with an equimolar concentration of human Vγ9-Vδ2 TCR in a deuterated buffer. Human Vγ9-Vδ2 TCR protein alone served as the control. Extent of protection was inferred by measuring the differences in hydrogen/deuterium exchange between Vγ9-Vδ2 TCR alone or in complex with the 7A5. The protection map and further refinements of the analysis showed significant protection of regions identified as residues: L₄₉VSISYDGTVRKESGIPSGK₆₈ (SEQ ID NO:774) on human Vγ9-Vδ2 TCR upon complexation with 7A5 (FIG. 27A). The epitope mapping by HXMS indicated that the 7A5 antibody primarily bound to a portion of CDR2 and FR3 in the Vγ9 chain of the TCR. The paratope of this antibody/antigen complex was also determined. Nearly all CDRs of 7A5 assist in making contribution to the Vγ9-Vδ2 TCR binding (FIG. 27B).

### Example 8.3. Vγ9⁺γδ T Cells are Suitable as Effector Cells for Redirection

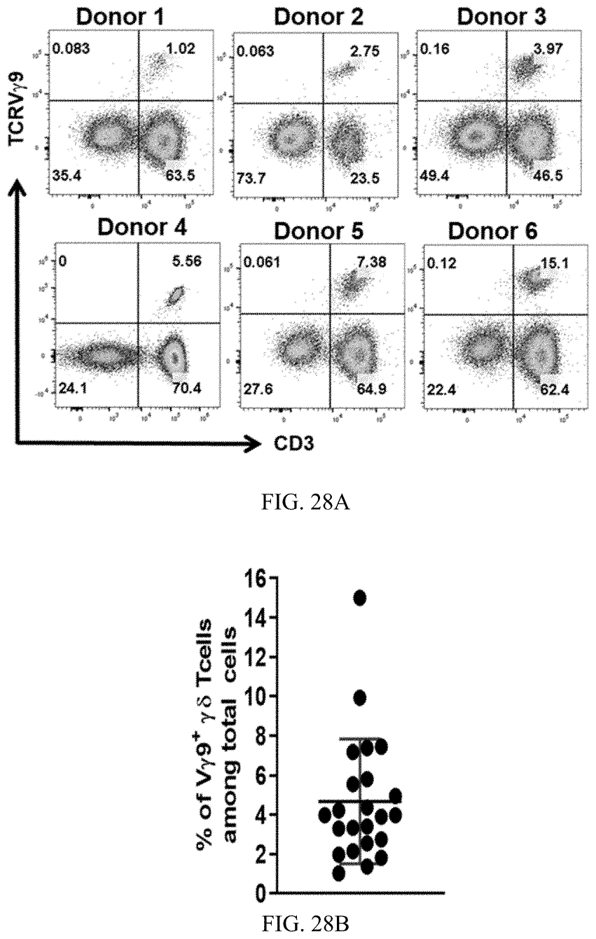

To recruit a specific subset of T cells to efficiently induce cytotoxicity of tumor cell targets, studies were focused on recruiting and activating circulating γδ T cells that express heterodimers of Vγ9/Vδ2 TCR chains because they manifest potent anti-cancer functions such as high cytotoxicity and interferon-γ secretion, see Kiladjian et al., Haematologica. 2008. 93:381-389. Moreover, agonist anti-Vγ9/Vδ2 TCR antibodies that bind to the Vγ9 (such as the clone 7A5) or Vδ2 chain was shown to activate the γδ T cells, see Wesselborg et al., J. Exp. Med. 1991. 173:297-304 and D'Asaro et al., J. Immunol. 2010. 184:3260-3268. Thus, the frequency of circulating Vγ9+ γδ T cells in the blood from multiple donors was determined. Data presented in FIG. 28A showed presence of varying populations of Vγ9+ γδ T cells ranging from approx. 1 to 15% of total CD3+ T cells analyzed from 6 human donors. Data presented in FIG. 28B showed presence of average of approximately 4% of Vγ9+ γδ T cells among total T cells when a large cohort of human subjects is analyzed.

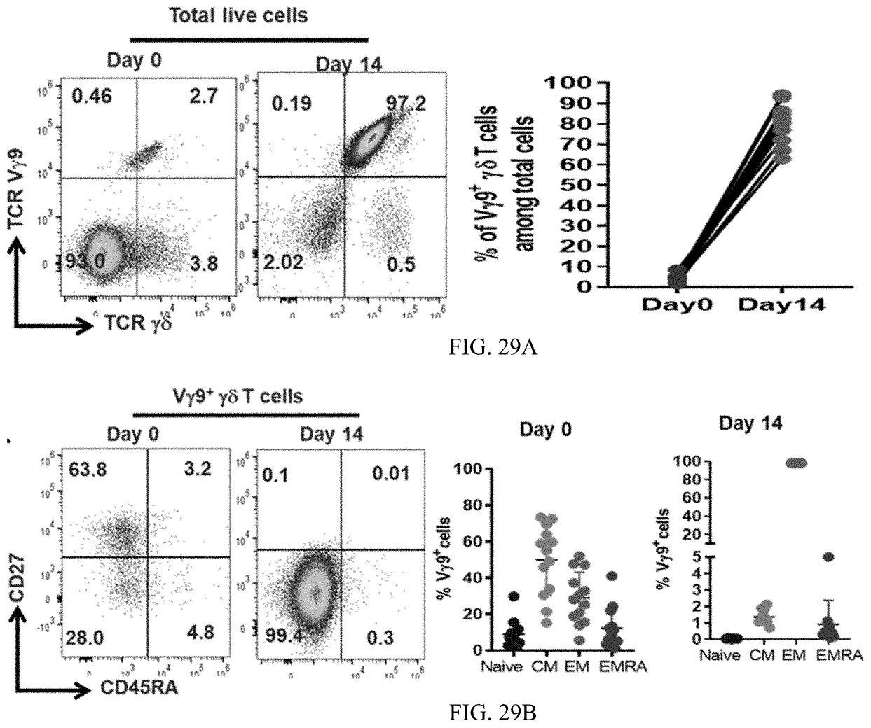

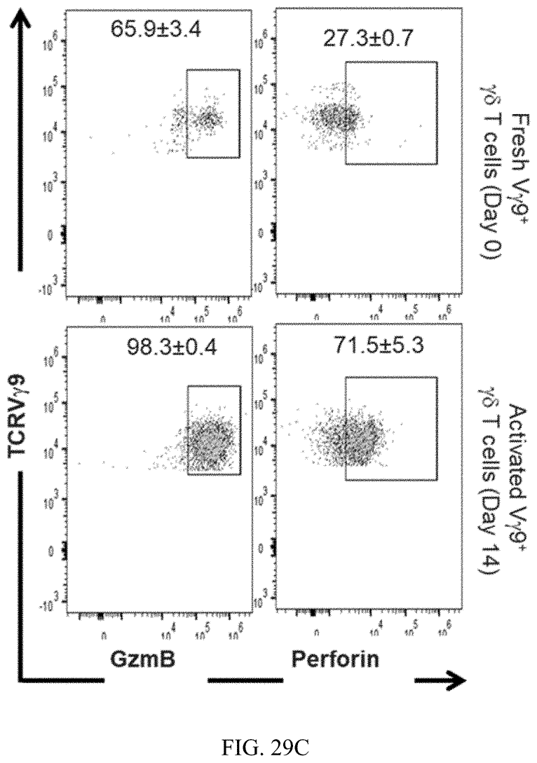

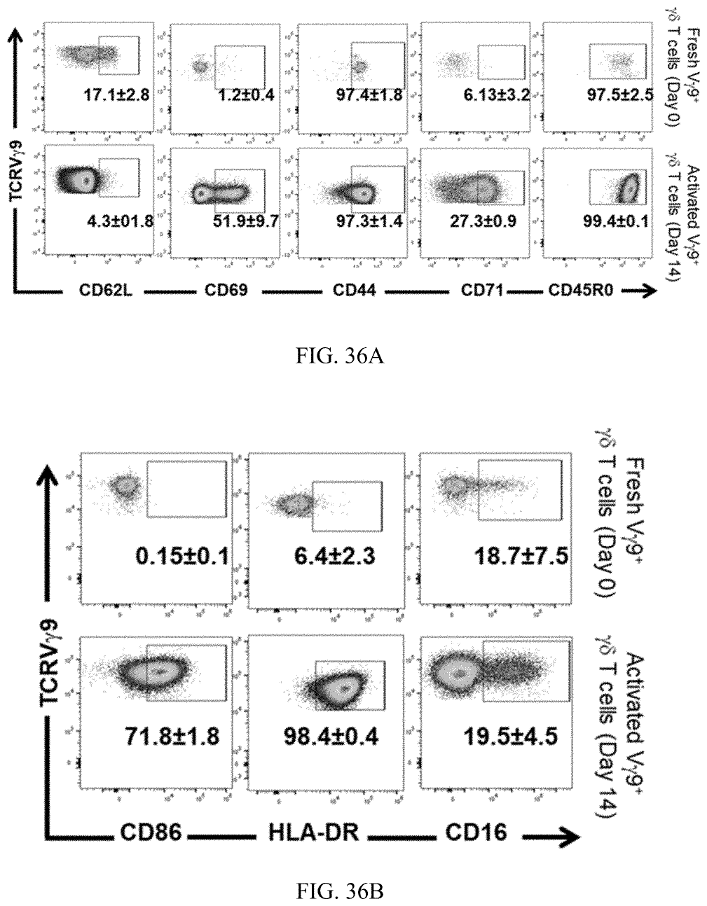

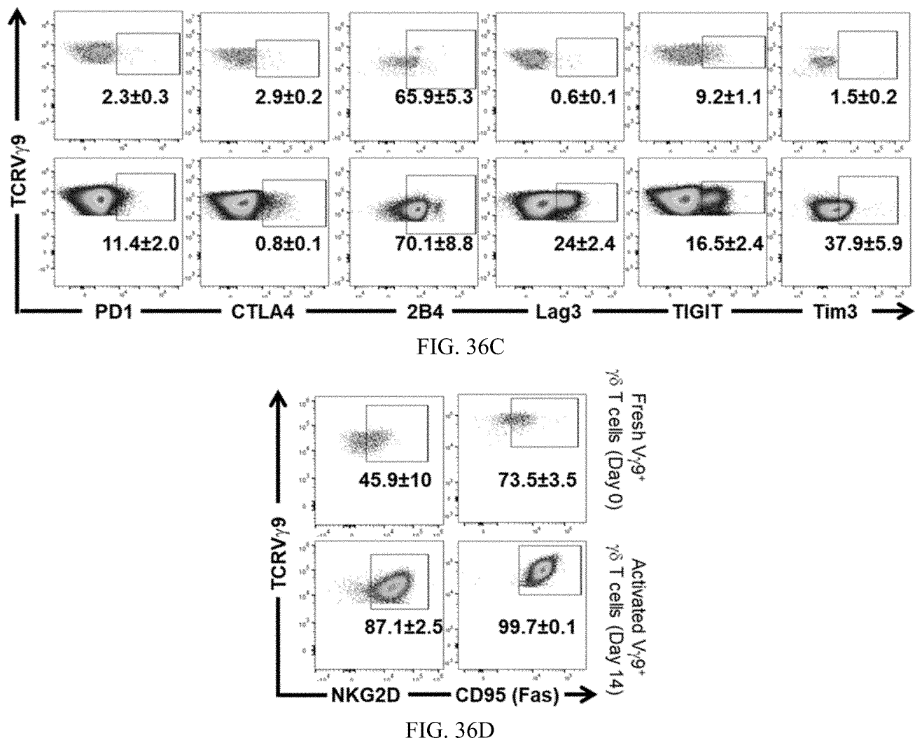

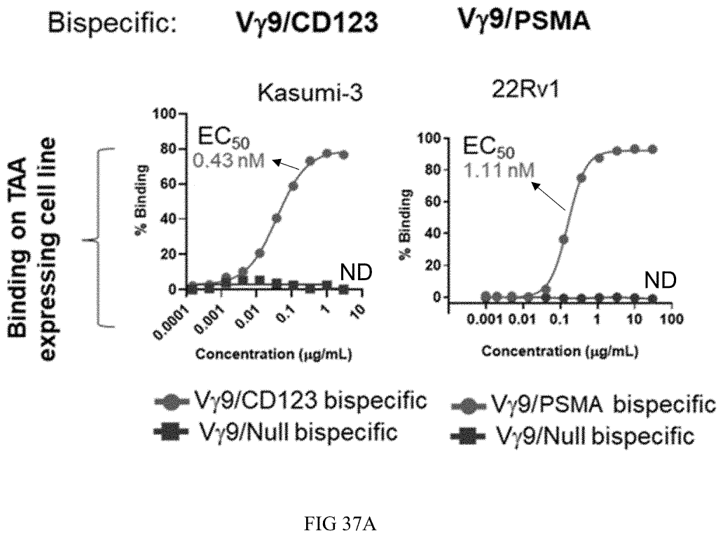

The phenotype of Vγ9+ γδ T cells was further analysed and determined to establish that they are suitable for redirecting to kill tumor cells. Zoledronic acid was used that selectively expands Vγ9+ γδ T cells from whole PBMCs as described in Example 8.1. Data presented in FIG. 29A showed Vγ9+ γδ T present in fresh PBMCs when cultured ex vivo with Zoledronic acid for 14 days expand to over 50-fold upon activation. Majority of the Vγ9+ γδ T cells present in fresh PBMCs are either central memory (CD27+, CD45RA−) or effort memory (CD27⁻, CD45RA⁻) whereas, activated and expanded cells also develop into effector memory cells (CD27−, CD45RA−) (FIG. 29B). The frequency of Vγ9+ γδ T cells expressing intracellular Granzyme B and Perforin from fresh PBMCs (upper row) and increased when PBMCs were cultured with Zoledronic acid 14 days (FIG. 29C). Collectively these data showed that abundance of Vγ9+ γδ cells in the circulation, and phenotype of activated Vγ9+ γδ T cells is appropriated for redirecting these cells to kill tumor cells. Additional data showing production of pro-inflammatory cytokines, upregulation expression of activation markers and low expression of markers of exhaustion as presented in FIGS. 36A-D again show that Vγ9+ subset of γδ T cell are suitable for redirecting for tumor elimination.

### Example 8.4 Anti-TRGV9/Anti-CD123 Bispecific Antibody Selectively Binds to Vγ9⁺ γδ T Cells and CD123 Expressing Tumor Cells

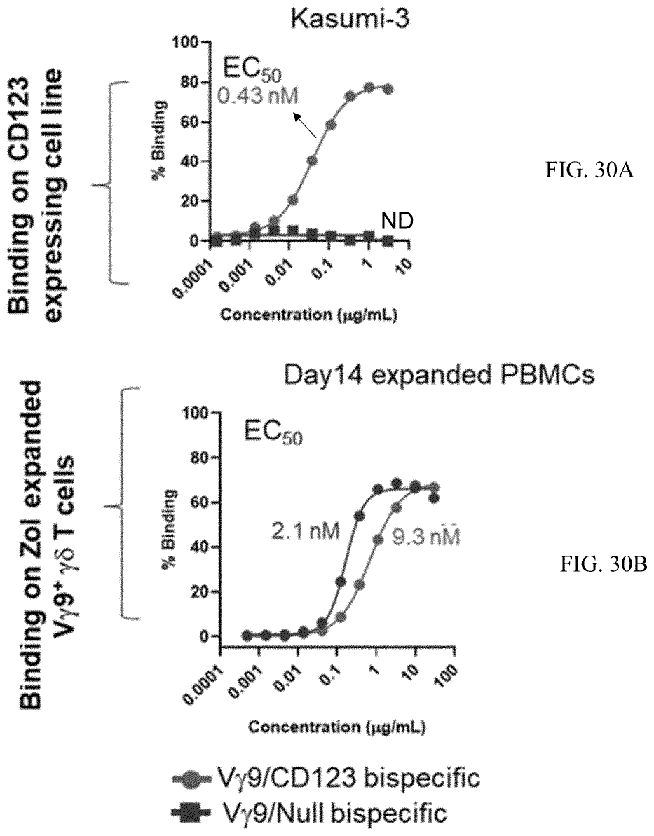

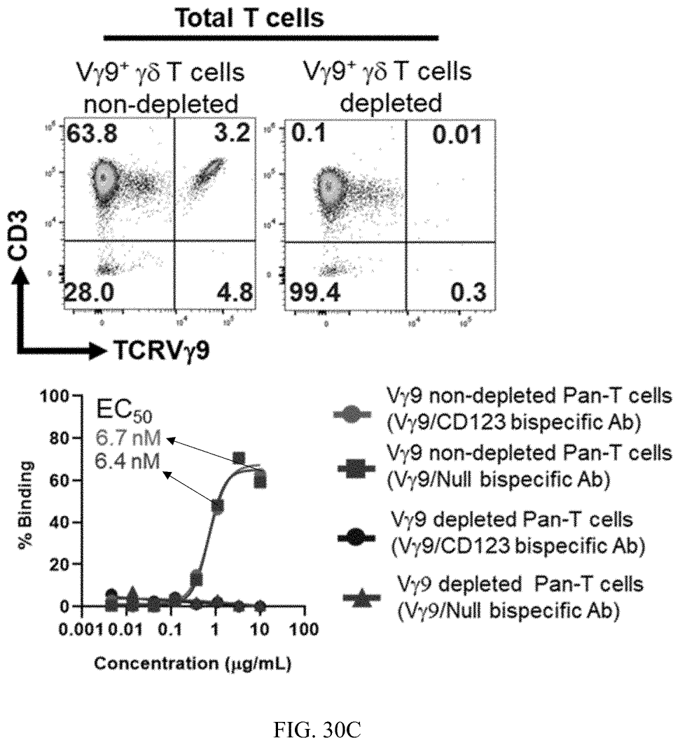

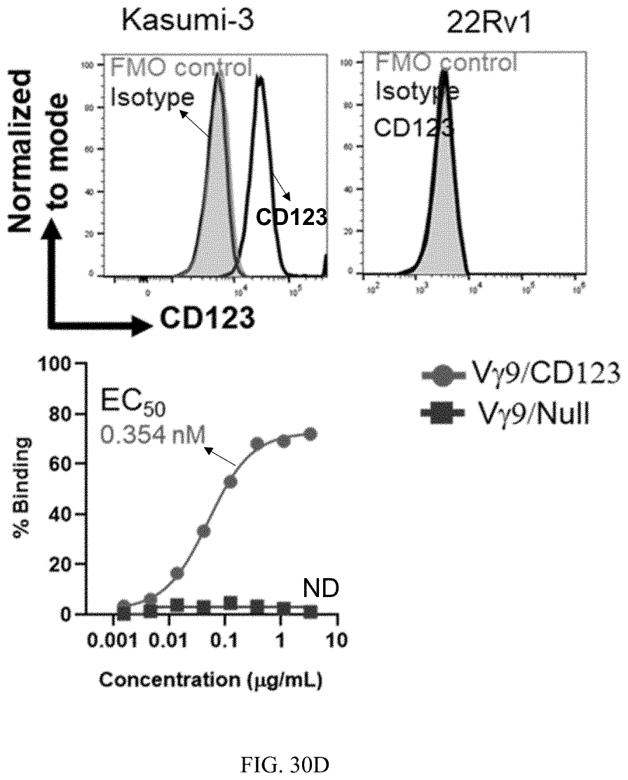

In order to determine binding of bispecific antibodies to Vγ9⁺γδ T cells and CD123 expressing tumor cells, CD123 expressing Kasumi-3 tumor target cells were incubated and 14 days Zoledronic acid expanded Vγ9+ γδ T cells in the presence or absence of indicated anti-TRGV9/anti-CD123 bispecific and respective Null arm control bispecific antibody and bound bispecific antibody staining was assessed as described in Example 8.1. The data showed binding of anti-TRGV9/anti-CD123 bispecific antibody and lack of binding of Null control bispecific antibody to CD123 expressing Kasumi-3 cells (FIG. 30A). Similarly, the binding of anti-TRGV9/anti-CD123 and Null control bispecific antibodies to Zoledronic acid was also determined to have expanded Vγ9+γ6 T cells. The data presented in FIG. 30B indicated binding of both anti-TRGV9/anti-CD123 and Null control bispecific antibodies confirming that bispecific antibodies are suitable molecules for recruiting Vγ9+ γδ T cells to CD123 expressing target cells. It was further determined if anti-TRGV9/anti-CD123 bispecific antibody selectively binds to Vγ9+ γδ T cells and CD123 expressing cell line. Vγ9+ γδ T cells were FACS-sort depleted from enriched Pan-T cells of whole PBMCs from healthy individuals. Total Pan-T cells and Pan-T cells depleted of Vγ9+ γδ T cells were incubated in the presence and absence of indicated bispecific antibodies at various concentrations. Representative FACS plots show the depletion efficacy of Vγ9+ γδ T cells among pan-T cells (FIG. 30C). Binding of Vγ9/CD123 and Vγ9/NULL bispecific antibodies respectively at indicated concentrations to pan-T cells and pan-T cells depleted of Vγ9⁺γδ T cells were determined and presented in the bottom panel (FIG. 30C). Collectively, these data demonstrate selective binding of anti-TRGV9/anti-CD123 and anti-Vγ9/Null bispecific antibodies to Vγ9⁺γδ T cells and lack of binding to Vγ9-negative pan-T cells.

It was further determined if anti-TRGV9/anti-CD123 bispecific antibody selectively binds to CD123 expressing cell line. CD123 expressing Kasumi-3 and non-expressing 22Rv1 cell lines were stained with anti-CD123 monoclonal antibody to show expression of CD123 (FIG. 30D upper panels). Data presented in FIG. 30D lower panels showed the binding of anti-TRGV9/anti-CD123 bispecific antibody to Kasumi-3 cells and lack of binding to 22Rv1 cell line suggesting binding of bispecific antibody is specific to CD123. These data demonstrate selective binding of anti-TRGV9/anti-CD123 bispecific antibodies to CD123+ tumor cells.

### Example 8.6 Anti-Trgv9/Anti-CD123 Bispecific Antibody Recruits Vγ9+ γδ T Cells into Biphasic Cell-Cell Conjugate Formation with Tumor Cells

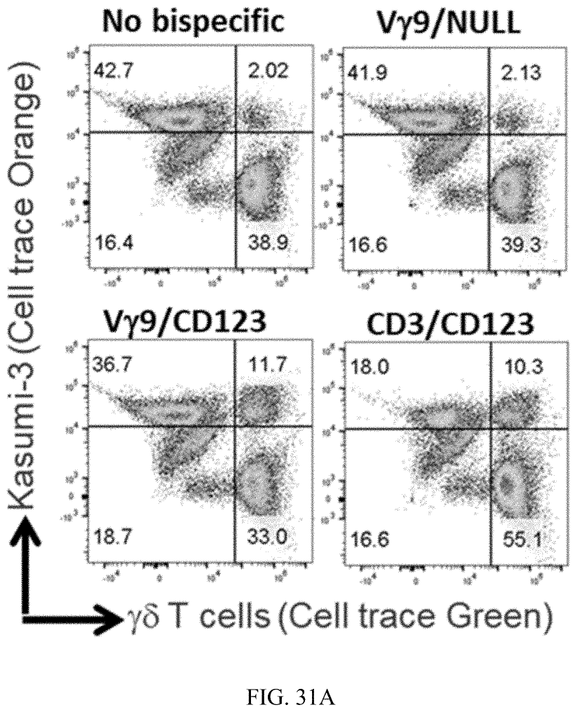

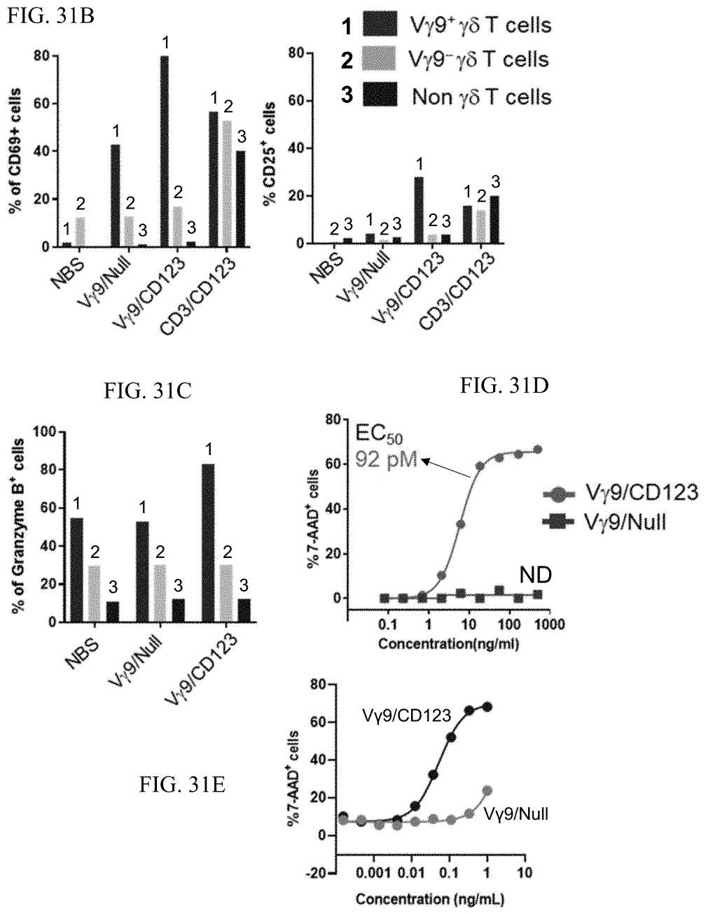

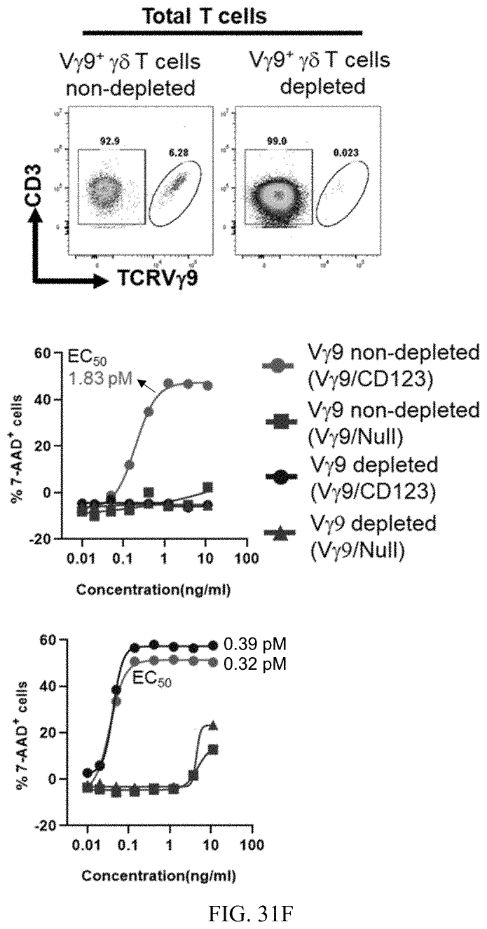

In order to determine if anti-TRGV9/anti-CD123 bispecific antibody recruits Vγ9+ γδ T cells into biphasic cell-cell conjugate. γδ T cells (effector cells) and Kasumi-3 (Targets) cells were co-cultured at an E:T ratio of 1:1 with 1 μg/ML of bispecific antibodies (anti-TRGV9/anti-CD123, anti-TRGV9/anti-NULL) and incubated at 37° C. for 1 hour. At the end of the incubation cells were analyzed utilizing the flow cytometer analysis software for conjugation formations as described in Example 8.1. Data presented in FIG. 31A showed that anti-TRGV9/anti-CD123 bispecific antibody mediated conjugation formation between Vγ9+ γδ T cells and CD123 expressing tumor cells similar to the conjugation formation by anti-CD3/anti-CD123 bispecific antibody. Taken together, these data suggest effective conjugation formation between effector and targets cells that is prerequisite for T cell mediated cytotoxicity.

### Example 8.7 Anti-Vγ9/CD123 Bispecific Selectively Activates Vγ9+ γδ T Cells and Mediate their Cytotoxicity

In order to determine selective activation of Vγ9+ γδ T cells and induction of their cytotoxicity, a system where whole fresh PBMCs were co-cultured with Kasumi-3 cells in the presence of various concentrations of the anti-TRGV9/anti-CD123 bispecific antibody was utilized. To serve as a positive and negative control, co-cultured cells were also stimulated with anti-CD3/anti-CD123 and anti-TRGV9/anti-NULL bispecific antibodies as described in Example 8.1. At the end of the culture frequency of Vγ9⁺ and Vγ9+ γδ T cells and non-γδ T cells positive for activation markers, CD69 (FIG. 31B left panel) and CD25 (FIG. 31B right panel) surface expression, and intracellular Granzyme B (FIG. 31C) expression were determined. The data demonstrated that Vγ9/CD123 bispecific selectively recruited and activated only Vγ9+ γδ T cells, whereas anti-CD3/anti-CD123 bispecific antibody affected pan-T cells and mediated their activation.

To further demonstrate that anti-TRGV9/anti-CD123 bispecific antibody mediates γδ T cell cytotoxicity against CD123 expressing Kasumi-3 cells in vitro, Vγ9+γδ T cells (Effectors) isolated from PBMCs expanded with Zoledronic acid for 14 days, were co-cultured with Kasumi-3 cells (Targets) in the presence of various concentrations of the bispecific antibody for 24 hours as described in Example 8.1. The data showed that anti-TRGV9/anti-CD123 bispecific antibody mediated Vγ9+ γδ T cell cytotoxicity against CD123 expressing kasumi-3 cells in a dose dependent manner (FIG. 6d). We next assessed Vγ9+ γδ cells from an AML patient to study cytotoxicity against CD123 expressing kasumi-3 cells. The data showed that anti-TRGV9/anti-CD123 bispecific antibody mediated efficient cytotoxicity against CD123 expressing kasumi-3 cells when Vγ9+ γδ T cells were obtained from AML patients (FIG. 31E). To strengthen the concept further that anti-TRGV9/anti-CD123 bispecific antibody selectively mediated cytotoxicity by engaging only Vγ9+ γδ T cells, Vγ9+ γδ T cells were FACS-sorted and depleted from Pan-T cells of whole PBMCs. Total Pan-T cells and Pan-T cells depleted of Vγ9+ γδ T cells were incubated in the presence and absence of indicated bispecific antibodies at various concentrations and Kasumi-3 tumor cells. Representative FACS plots show the depletion efficacy of Vγ9+ γδ T cells among pan-T cells (FIG. 31F upper panels). Cytotoxicity of Vγ9/CD123 bispecific antibody at indicated concentrations with pan-T cells and pan-T cells depleted of Vγ9+ γδ T cells were determined and presented in the middle panel (FIG. 31F), where no cytotoxicity was seen when Vγ9+ γδ T cell were depleted. On the other hand, cytotoxicity of CD3/CD123 bispecific antibody with pan-T cells and pan-T cells depleted of Vγ9+ γδ T cells was evident. These data, thus, showed that anti-TRGV9/anti-CD123 bispecific antibody can selectively activate and recruit Vγ9+ γδ T cells to effectively kill tumor cells bearing CD123, and demonstrated that Vγ9+ γδ T cells from AML patients can also be redirected to kill tumor cells.

### Example 8.8 Anti-TRGV9/Anti-CD123 Bispecific Antibody Potently Mediates Activation, Proliferation and Cytotoxicity by Vγ9+ γδ T Cells Among Whole PBMCS

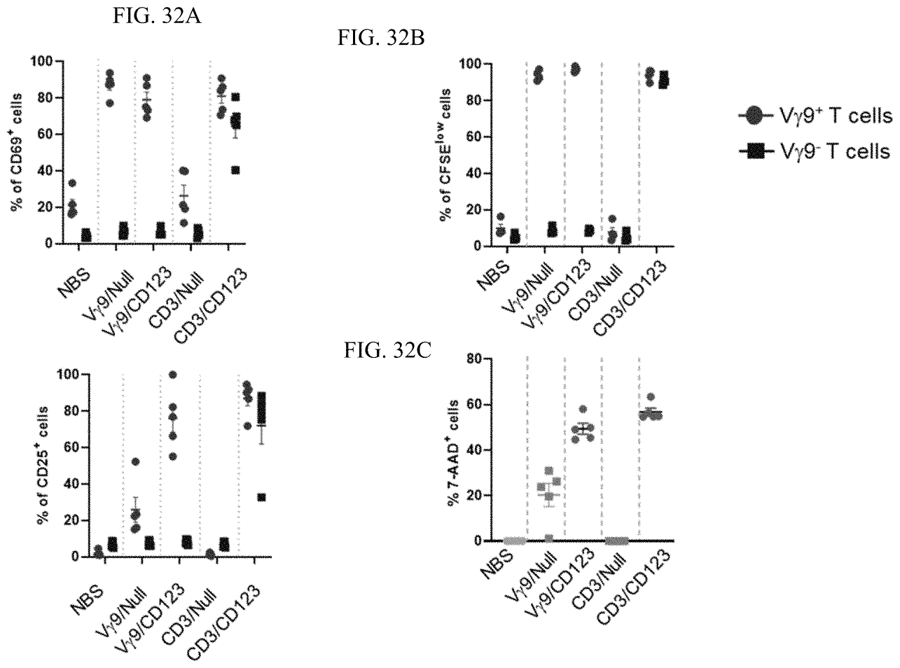

In order to specifically determine if anti-TRGV9/anti-CD123 bispecific antibody selectively activate only Vγ9+ γδ T cells, whole PBMCs were co-cultured with CD123 expressing targets (kasumi-3 cells) together with anti-TRGV9/anti-CD123 or anti-CD3/anti-CD123 along with control bispecific antibodies as described in Example 8.1. Frequency of Vγ9+ or Vγ9+ γδ T cells (Pan T cells lacking Vγ9+ γδ T cells) among CD3+ T cells expressing activation markers, CD69 and CD25 were determined by FACS analysis. Data presented in FIG. 32 showed induction of expression of CD69 (panel A) and CD25 (panel B) by anti-TRGV9/anti-CD123 bispecific antibody only in the Vγ9+ γδ T cell compartment, whereas anti-CD3/anti-CD123 activated both Vγ9+ and Vγ9− T cell population. Similarly, CFSE dilution as a measure of proliferation was also very selective for anti-TRGV9/anti-CD123 bispecific antibody, it only induced proliferation in Vγ9+ γδ T cells. Conversely, the anti-CD3/anti-CD123 bispecific antibodies induce proliferation in all T cells irrespective of Vγ9+ expression (FIG. 32B). We further looked at the ability of anti-TRGV9/anti-CD123 bispecific antibody to eliminate exogenously added Kasumi-3 target cells among whole PBMCs. The data suggested that although anti-TRGV9/anti-CD123 bispecific antibody only recruited and activated Vγ9+ γδ T cells (a fraction of total T cells in the PBMC population), it mediated target cell elimination, similar to anti-CD3/anti-CD123, which recruits and activates all pan-T cells (FIG. 32C). Taken together, the data suggested efficient and selective activation and induction of cytotoxicity mediated by anti-TRGV9/anti-CD123 bispecific antibody despite presence of only ˜4% Vγ9+ γδ T cells among Total T cells in PBMC population.

### Example 8.9: Vγ9+ γδ T Cell Selective Redirection does Not Elicit Cytokine Storm Compared to Pan-T Cell Re-Direction

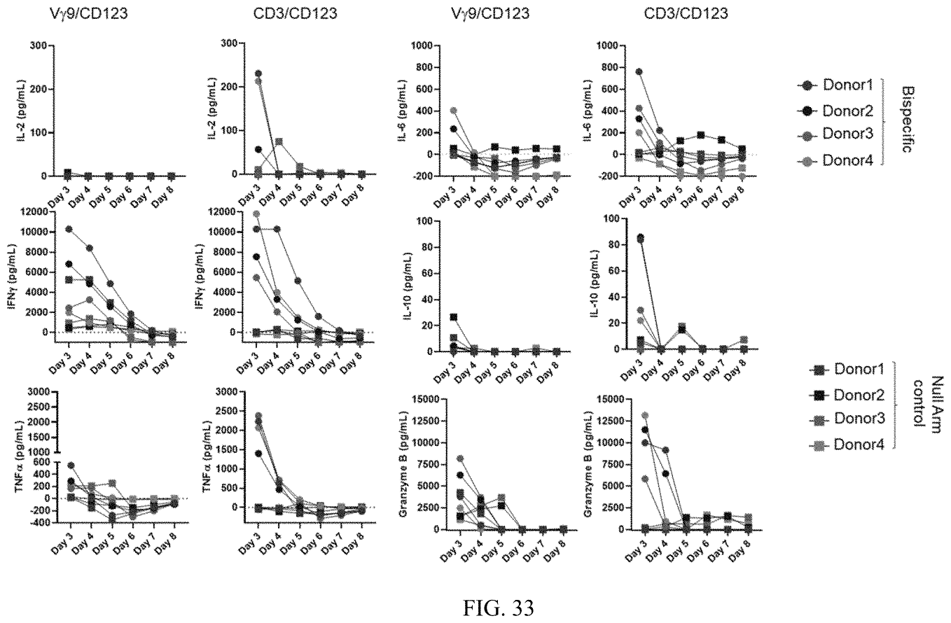

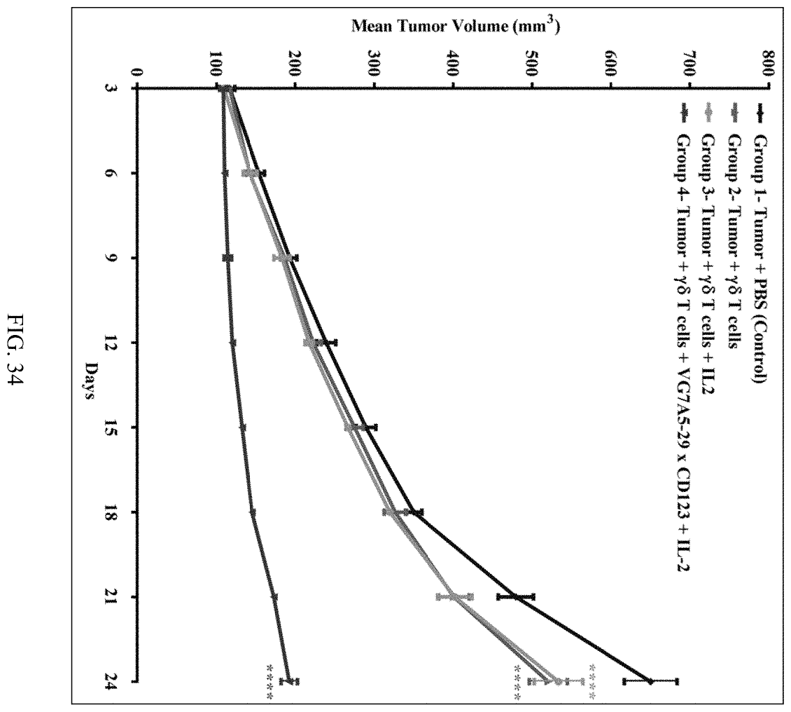

One of the major concerns of pan T cell activation and redirection is that it leads to severe cytokine storm that results in suboptimal efficacy and a narrow therapeutic index. The approach was to selectively redirect only Vγ9+ cytotoxic cells that are capable of lysing tumor cells rather than indiscriminately stimulating and recruiting pan-T cells and broadening therapeutic index. Although redirecting Vγ9+ γδ T cells results in efficient killing, it is not clear if these cells will also induce similar level of cytokines as with Pan-T cell recruitment. In order to address this question, anti-TRGV9/anti-CD123 bispecific antibody were compared with anti-CD3/anti-CD123 antibody induced cytokine production by whole PBMCs. Cultures were set up using whole PBMC in the presence of kasumi-3 cells and bispecific antibodies as described Example 8.1. From day 3 of culture to day 8 of culture supernatants were analyzed for cytokine production. Data presented in FIG. 33 indicated that anti-TRGV9/anti-CD123 bispecific antibody induced much lower cytokine production as compared to anti-CD3/anti-CD123 bispecific antibody. Notably, IL-6 and IL-10 levels that are believed to the main players for cytokine storm in patients undergoing CD3-redirection immunotherapy. Based on these data, Vγ9-redirection therapy is less likely to induce cytokine storm than CD3-based bispecifics and thus, it may help to broaden therapeutic index.

### Example 8.10: Anti-TRGV9/Anti-CD123 Bispecific Antibody Effectively Controlled Kg1 Tumor Cell Growth in A Xenograft Model

NOD SCID mice were subcutaneously injected with KG-1 cells as described in detail in Example 8.1. Two days post tumor implant, mice were randomized based on the body weight and segregated into 4 groups. After the randomization (Day 2), subcutaneous injection of either Vγ9+ γδ T cells or 15 μg/kg IL-2 with PBS or 1.5 mg/kg (VG7A5-29×CD123) was administered. Tumor volume (mm³) was measured at indicated time points as mentioned in Example 8.1. The data presented in FIG. 34 indicated that selective recruitment of γδ T cells by VG7A5-29×CD123 bispecific antibody in presence of IL2 showed higher tumor growth inhibition and efficacy. The percentage of tumor growth inhibition on day 24 is represented in Table 38 below. These data further suggest that selective redirection of a small subset of T cells redirection approach in mediating tumor cell cytotoxicity in vivo.

TABLE 38
Percentage of Tumor Growth.

| | | Tumor Volume | % Tumor growth |
|---|---|---|---|
| | | (mm³) day 24 | inhibition on day |
| Group | Treatment and Dose | mean ± SEM | 24 (by delta) |
| Group 1 | Tumor + PBS (control) | 650 ± 33 | — |
| Group 2 | Tumor + γδ T cells | 521 ± 24 | 24 |
| Group 3 | Tumor + γδ T cells + IL2 | 533 ± 31 | 21 |
| Group 4 | Tumor + γδ T cells + | 193 ± 10 | 84 |
| | VG7A5-29 × CD123 + IL2 | | |

### Example 9—Binding Characterization and Activity of Various TRGV9 Multispecific Antibodies

Additional experiments were conducted to further determine the activation profiles of various TRGV9 antibodies provided herein.

### Example 9.1: Binding of TRGV9 Antibodies to Vγ9

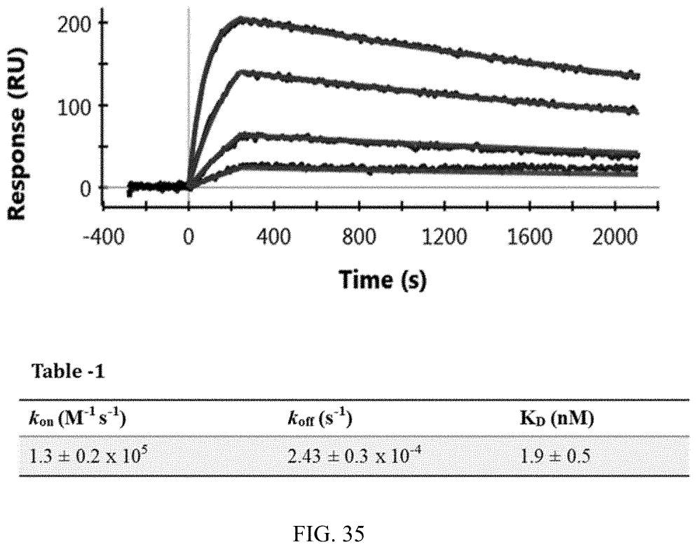

FIG. 35 shows binding kinetics of mouse anti-human TCR Vγ9 [clone 7A5] and recombinant Vγ9-Vδ2-Fc antigen by SPR at 25° C. Different concentrations of antigen (100 nM, from top to bottom in the plot) were flowed through anti-Vγ9 mAb that was captured on the surface. Experimental data (black line) and 1:1 Langmuir binding fitting (red line) is shown. The association phase between (first ˜250 sec) is follow by the dissociation phase. Global fitting to a 1:1 simple Langmuir binding model resulted kₒₙ=1.3±0.2×10⁵M⁻¹ S⁻¹ and k_{off}=2.43±0.3×10⁻⁴ S⁻¹ giving a K_{D}=1.9 nM.

### Example 9.2: Suitability of Vγ9+ Subsets of γδ T Cells for Anti-TRGV9-Mediated Redirection and Tumor Elimination

FIGS. 36A-36D show that Vγ9+ subset of γδ T cell are suitable for redirecting for tumor elimination. FIG. 36A shows the frequency (mean±SEM) of ζγ9+ γδ T cells on day 0 (top row) and day 14 (bottom row) positive for activation markers. FIG. 36B shows an antigen presenting cell characteristics. FIG. 36C shows exhaustion markers. FIG. 36D shows NK markers/characteristics. Representative data on n=7 donors for CD62L, CD69, CD44, 2 donors for CD45R0 and CD71, 3 donors for CD86, HLA-DR and CD16, 5 donors for NKG2D, 3 donors for CD95 (Fas) surface expression on day 0 Vγ9+ γδ T cells. n=8 donors for CD62L, 9 donors for CD69 and CD44, 5 donors for CD45Ro and CD71, 3 donors for CD86, HLA-DR, CD16, 14 donors for NKG2D, 6 donors for CD95 (Fas) surface expression on activated Vγ9+ γδ T cells (day 14 n=13 donors for PD1 and Lag3, 5 donors for CTLA4 and 2B4, 4 and 7 donors for TIGIT and Tim3 surface expression respectively on fresh Vγ9+ γδ T cells (day 0). n=16 donors for PD1, 5 donors for CTLA4 and 2B4, 13 donors for Lag3, 14 donors for TIGIT surface expression on activated Vγ9+ γδ T cells (day 14). >5 individual experiments were carried out.

### Example 9.3: Preparation of Anti-TRGV9/Anti-PSMA Multispecific Antibodies

Using methods described elsewhere herein, the variable region sequences of anti-TRGV9 and anti-PSMA antibodies were used to generate a bispecific TRGV9×PSMA antibody to be tested for Vγ9+ γδ T cell activation and re-directed killing of target cells that express PSMA (see Example 9.4). Provided below is a summary of the VH and VL CDRs (Table 39), as well as the VH, VL heavy chain and light chain amino acid sequences (Table 40) of the PSMA clone (PSMB365) utilized for these experiments, which was prepared as provided in U.S. Publ. No. 2020/0048349.

TABLE 39
PSMA Antibody VH and VL CDR Sequences
PSMB365

| PSMH865 | | | PSML160 | | |
|---|---|---|---|---|---|
| VH CDR1 | VH CDR2 | VH CDR3 | VL CDR1 | VL CDR2 | VL CDR3 |
| SDAMH | EISGSGGYT | DSYDSSLY | RASQSVS | DASYRAT | QQRRNWPL |
| (SEQ ID | NYADSLKS | VGDYFDY | SYLA | (SEQ ID | T (SEQ |
| NO: 783) | (SEQ ID | (SEQ ID | (SEQ ID | NO: 787) | ID NO: |
| | NO: 784) | NO: 785) | NO: 786) | | 788) |

TABLE 40
PSMA Antibody VH, VL, HC and LC Sequences.

| | | | |
|---|---|---|---|
| PSMB365 | VH | PSMH865 | EVQLLESGGGLVQPGGSLRLSCAASGFTFKSDAMHWVRQAPGKGL |
| | | | EWVSEISGSGGYTNYADSLKSRFTISRDNSKNTLYLQMNSLRAED |
| | | | TAVYYCARDSYDSSLYVGDYFDYWGQGTLVTVSS |
| | | | (SEQ ID NO: 775) |
| | VL | PSML160 | EIVLTQSPATLSLSPGERATLSCRASQSVSSYLAWYQQKPGQAPR |
| | | | LLIYDASYRATGIPARFSGSGSGTDFTLTISSLEPEDFAVYYCQQ |
| | | | RRNWPLTFGQGTKVEIK |
| | | | (SEQ ID NO: 776) |
| | HC | | EVQLLESGGGLVQPGGSLRLSCAASGFTFKSDAMHWVRQAPGKG |
| | | | LEWVSEISGSGGYTNYADSLKSRFTISRDNSKNTLYLQMNSLRA |
| | | | EDTAVYYCARDSYDSSLYVGDYFDYWGQGTLVTVSSASTKGPSV |
| | | | FPLAPCSRSTSESTAALGCLVKDYFPEPVTVSWNSGALTSGVHT |
| | | | FPAVLQSSGLYSLSSVVTVPSSSLGTKTYTCNVDHKPSNTKVDK |
| | | | RVESKYGPPCPPCPAPEAAGGPSVFLFPPKPKDTLMISRTPEVT |
| | | | CWVDVSQEDPEVQFNWYVDGVEVHNAKTKPREEQFNSTYRVVSV |
| | | | LTVLHQDWLNGKEYKCKVSNKGLPSSIEKTISKAKGQPREPQVY |
| | | | TLPPSQEEMTKNQVSLTCLVKGFYPSDIAVEWESNGQPENNYKT |
| | | | TPPVLDSDGSFFLYSRLTVDKSRWQEGNVFSCSVMHEALHNHYT |
| | | | QKSLSLSLGK |
| | | | (SEQ ID NO: 781) |
| | LC | | EIVLTQSPATLSLSPGERATLSCRASQSVSSYLAWYQQKPGQAP |
| | | | RLLIYDASYRATGIPARFSGSGSGTDFTLTISSLEPEDFAVYYC |
| | | | QQRRNWPLTFGQGTKVEIKRTVAAPSVFIFPPSDEQLKSGTASV |
| | | | VCLLNNFYPREAKVQWKVDNALQSGNSQESVTEQDSKDSTYSLS |
| | | | STLTLSKADYEKHKVYACEVTHQGLSSPVTKSFNRGEC |
| | | | (SEQ ID NO: 782) |

### Example 9.4: TRGV9×CD123 or Trgv9×PSMA Multispecific Antibody Binding to TAA-Expressing Tumor Cells

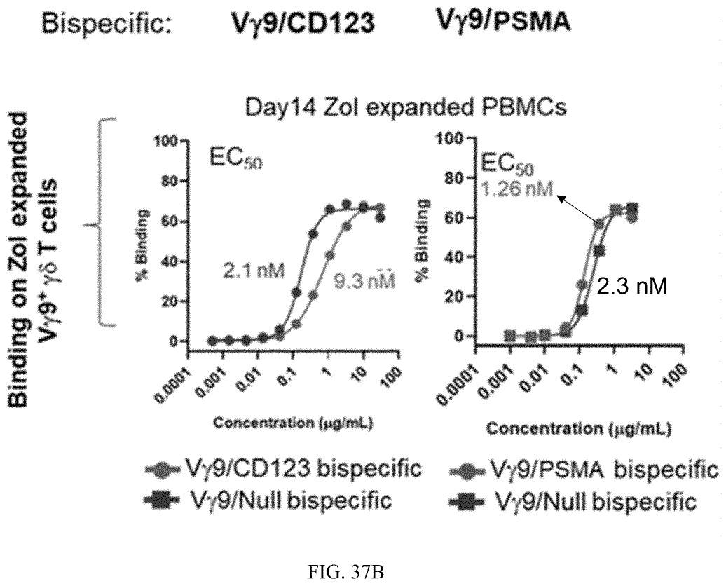

FIGS. 37A-37B show Vγ9 bispecific binding to Vγ9⁺γδ T cells and TAA expressing tumor cells. Tumor Associated Antigen (TAA) expressing target cells and day 14 Zol expanded PBMCs were incubated in the presence or absence of indicated Vγ9 bispecific (Vγ9/CD123 or Vγ9/PSMA) and Null arm control bispecific antibodies. Bound bispecific antibody staining was assessed by flow cytometry. Representative graphs show the frequency of Vγ9 bispecific bound cells at various concentrations. Vγ9 bispecific antibody and its corresponding Vγ9 null arm bispecific control antibodies are indicated. EC₅₀ values shown in the graph were derived using a 4-parameter dose-response curve with the concentration of indicated bispecific antibody on the x-axis (log scale) and specific binding on the y-axis (linear scale). FIG. 37A shows representative data of n=3 independent experiments for Kasumi-3, 22Rv1 cell lines and FIG. 37B shows representative data of n=8 and 2 healthy donors from 2 independent experiments are shown for Vγ9/CD123 and Vγ9/PSMA bispecifics respectively. EC₅₀ values shown in the graphs refers to mean of 3 independent experiments (for FIG. 37A) and 9 and 2 healthy donors (for FIG. 37B).

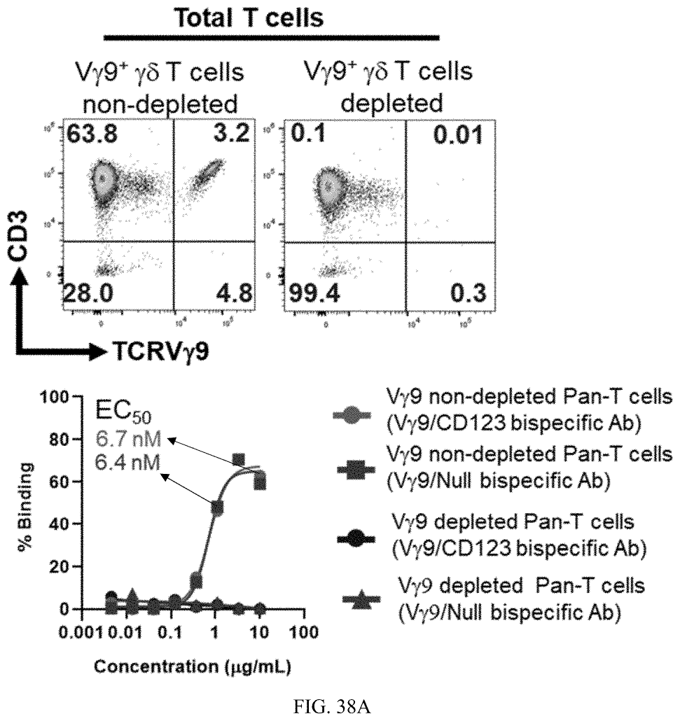

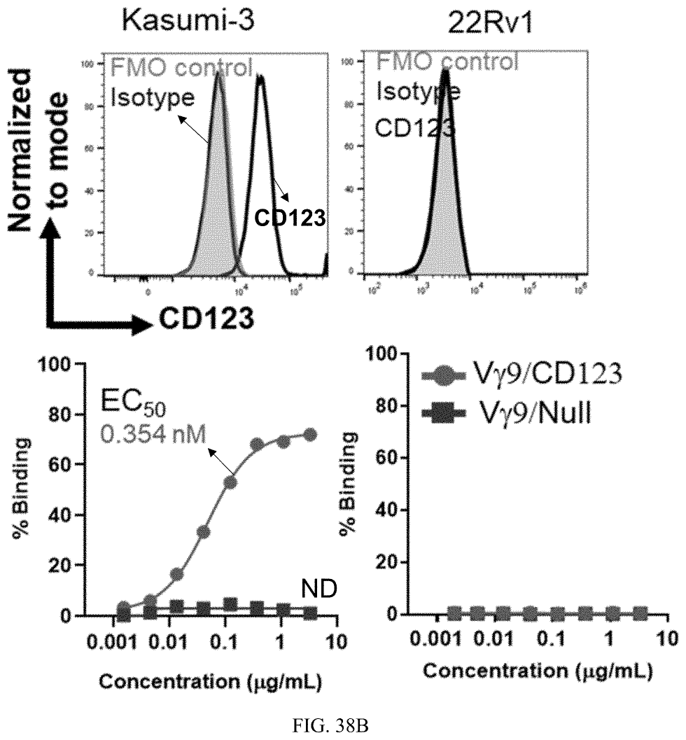

FIGS. 38A-38B show that Vγ9/CD123 bispecific selectively binds to Vγ9⁺ γδ T cells and CD123 expressing cell line. Vγ9⁺γδ T cells were FACS-sort depleted from enriched Pan-T cells of whole PBMCs from healthy individuals. Total Pan-T cells and Pan-T cells depleted of Vγ9⁺γδ T cells were incubated in the presence and absence of indicated bispecific antibodies at various concentrations. FIG. 38A depicts representative FACS plots showing the depletion efficacy of Vγ9⁺ γδ T cells among pan-T cells. Numbers in quadrants represent the frequency of the respective population. FIG. 38A reflects the binding of Vγ9/CD123 and Vγ9/NULL bispecific antibodies respectively at indicated concentrations to pan-T cells and pan-T cells depleted of Vγ9⁺γδ T cells. CD123 TAA expressing Kasumi-3 and non-expressing 22Rv1 cell lines were stained with anti-CD123 monoclonal antibody. FIG. 38B shows the staining of CD123, isotype control and FMO control respectively on Kasumi-3 (left) and 22Rv1 (right) cell lines. FIG. 38B shows the binding of Vγ9/CD123 and Vγ9/NULL bispecific antibodies respectively to Kasumi-3 (left) and 22Rv1 (right) cells lines at indicated concentrations. EC₅₀ values shown in the graph were derived using a 4-parameter dose-response curve with the concentration of indicated bispecific antibody on the x-axis (log scale) and specific binding on the y-axis (linear scale).

### Example 9.5: TRGV9×CD123 or Trgv9×PSMA Multispecific Antibodies Effectively Redirect γδ T Cells and Eliminate Both Liquid and Solid Tumors

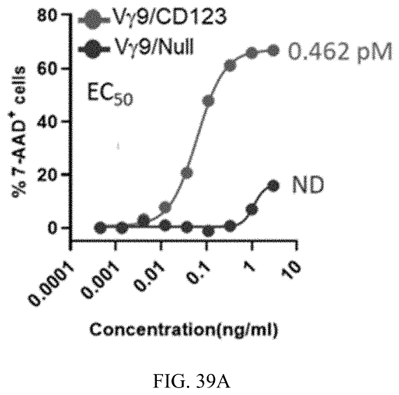

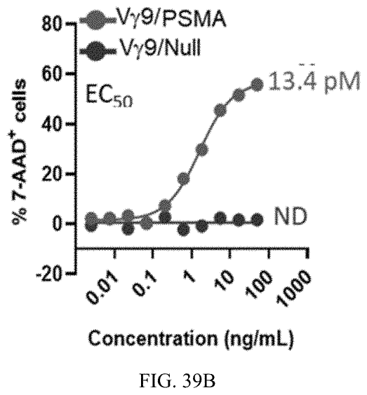

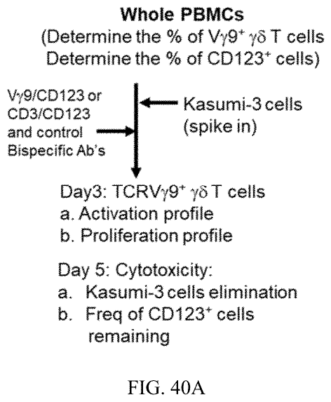

FIGS. 39A-39B show that Vγ9 bispecific mediated re-direction of γδ T cells effectively eliminates liquid and solid tumors. Whole PBMCs were cultured in the presence of Zol+IL-2+I1-15 for 14 days. Vγ9⁺γδ T cell frequency was assessed among whole PBMCs by flow cytometry. Day 14 Zol cultured PBMCs (effectors) were co-cultured with CFSE labelled target (Kasumi-3) cells at ET ratio 1:1 (for Kasumi-3 cells) and 5:1 (for 22Rv1) ET ratio (by normalizing ET ratio to Vγ9 frequency in Zol expanded PBMCs) in the presence of indicated concentration of Vγ9 bispecifics and Vγ9 NULL arm control antibodies for a period of 16 hours (for Kasumi-3 targets) and 72 hours (for 22Rv1) and at 37° C. in a humidified CO2 incubator. Target cell lysis was determined by the 7-AAD staining and flow cytometry. Graphs shown in FIG. 39A and FIG. 39B represent the frequency of specific target cell lysis at the indicated concentration of Vγ9 bispecific antibodies and their respective Vγ9/NULL arm controls. EC₅₀ values shown in representative graphs are mean of 8 and 2 healthy donors for Vγ9/CD123 (FIG. 39A) and Vγ9/PSMA (FIG. 39B) bispecific antibodies respectively from 3 (for FIG. 39A) and one (for FIG. 39B) independent experiments.

### Example 9.6: TRGV9×CD123 Multispecific Antibodies Mediate the Activation, Proliferation and Effector Function of Vγ9+ γδ T Cells

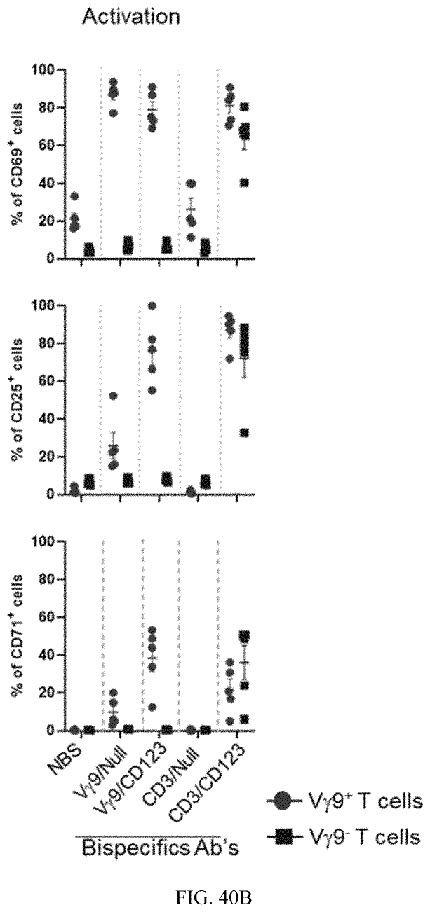

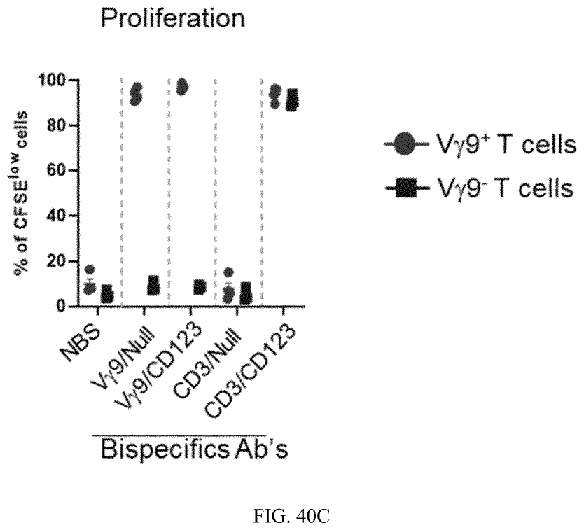

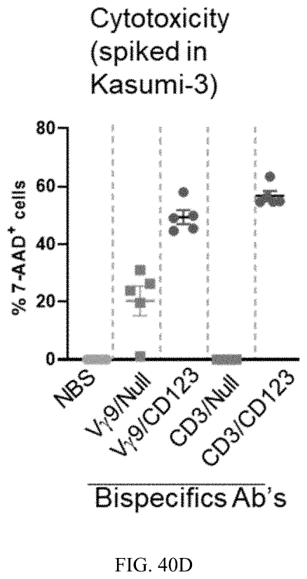

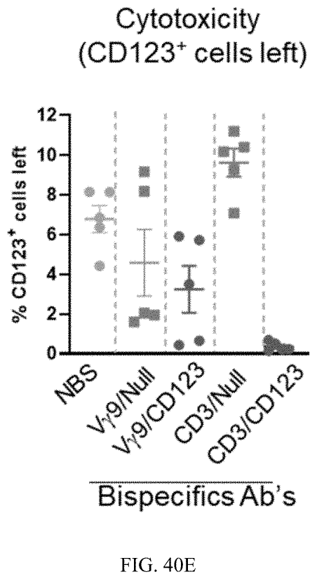

FIGS. 40A-40E show that Vγ9/CD123 bispecific antibody potently mediates activation, proliferation and effector functions of Vγ9⁺γδ T cells among whole PBMCs. FIG. 40A shows how CFSE labelled whole PBMCs were cultured in the presence or absence of spiked-in kasumi-3 cells in the presence of indicated bispecific antibodies at a concentration of 3 ng/mL. As a control, CFSE labelled whole PBMCs (with or without spiked in Kasumi-3 cells) were cultured in the absence of any bispecific antibody. FIG. 40B depicts graphs that represent the mean (±SEM) frequency of Vγ9⁺ cells positive for surface expression of CD69, CD25 and CD71 (activation markers). FIG. 40C shows CFSE dilution (proliferation profile) and FIG. 40D shows ability to eliminate exogenously added Kasumi-3 cells or endogenous CD123⁺ cells (as shown in FIG. 40E) among whole PBMCs (effector profile) upon culture in the absence or presence of indicated bispecific antibodies (FIGS. 40C-40E). Each dot represent data from an individual healthy donor. Representative data of n=5 donors from 2 independent experiments is shown in here.

### Example 9.7: Vγ9+ γδ T Cell Selective Redirection by Multispecific TRGV9 Antibodies does not Elicit Cytokine Storm

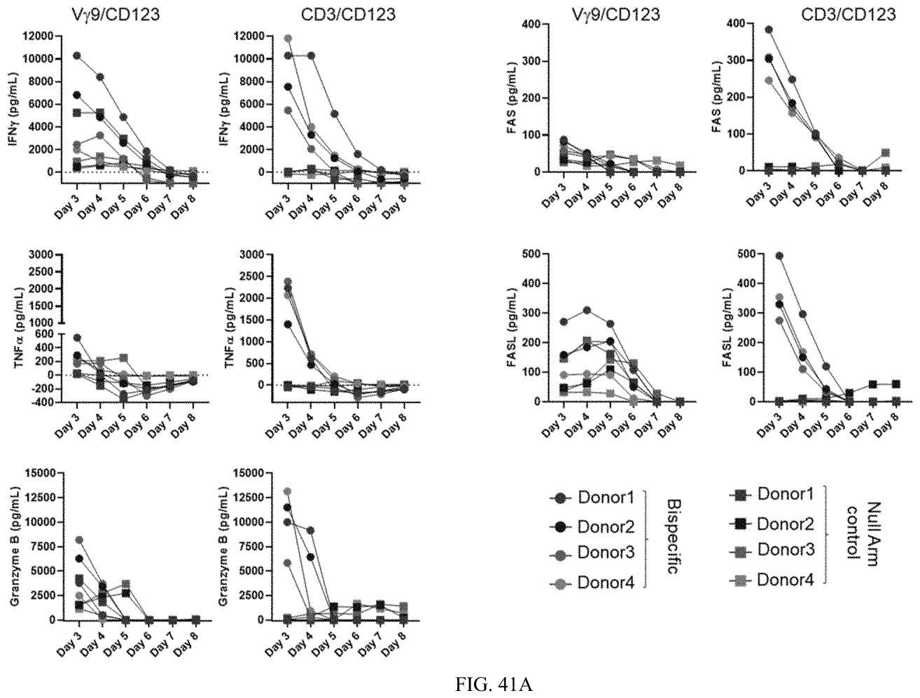

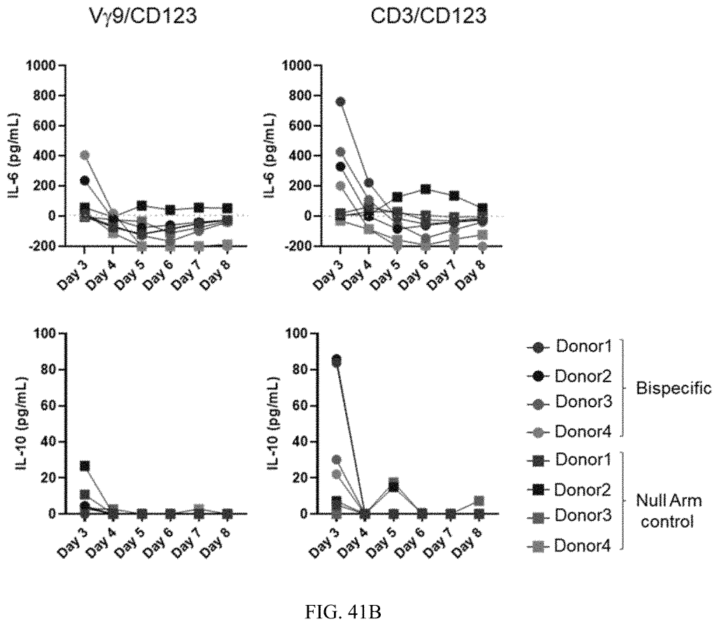

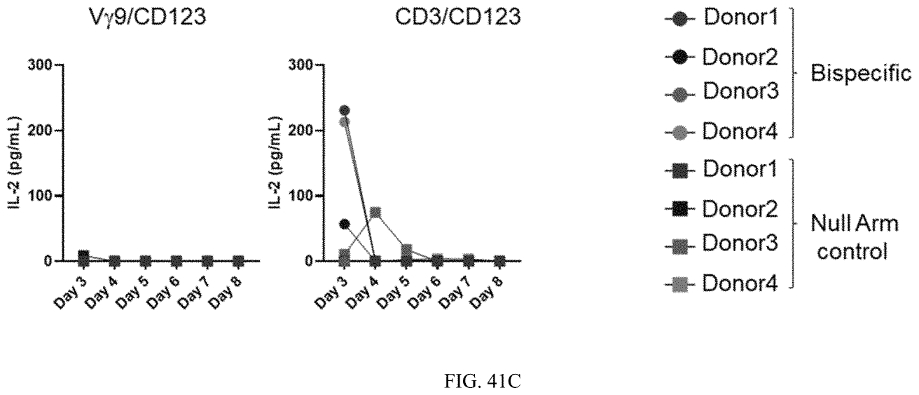

FIGS. 41A-41C show that Vγ9+ γδ T cell selective redirection does not elicit cytokine storm compared to Pan-T cell re-direction. Whole PBMCs were cultured in the presence or absence of spiked-in kasumi-3 cells in the presence or absence of indicated bispecific antibodies (3 ng/ml) as described in FIG. 40. From day 3 of culture onwards, 100 μL of culture medium was removed every day from the wells, without disturbing the cells, and replenished with fresh medium until day 8 of culture. Cytokines were assessed from day 3 to day 8 cell culture supernatant. FIG. 41A, FIG. 41B and FIG. 41C show concentration of various cytokines or effector molecules in the culture supernatant of whole PBMCs stimulated with indicated bispecific antibodies. Circles and squares represent PBMCs from four individual donors stimulated with indicated bispecific antibodies or NULL arm control bispecific antibodies respectively. Representative data of n=4 donors from one independent experiment is shown here.

### Example 9.8: Multispecific TRGV9 Antibodies Mediate γδ T Cell Proliferation and Redirection in PBMC of AML Patients

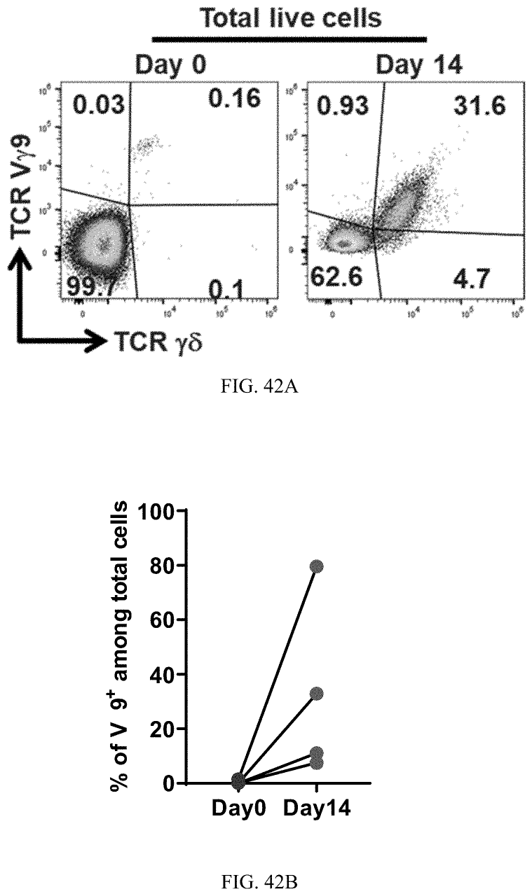

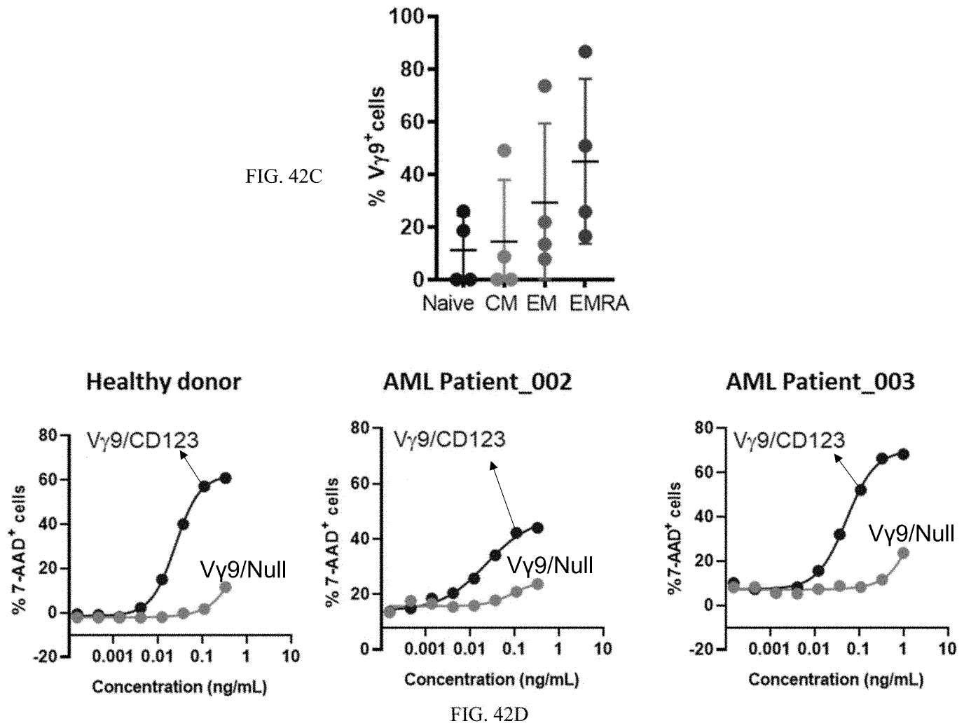

FIGS. 42A-42D show Vγ9/CD123 bispecific mediated γδ T cells redirection in AML patients PBMCs. FIG. 42A shows TCR Vγ9⁺γδ T cells from AML patients can be expanded via ZoL. Numbers in representative FACS plots show the frequency of Vγ9⁺ and Vγ9⁻ γδ T cells on day 0 (left) and day 14 (right) AML patients PBMCs culture with Zol+IL-2+IL-15. FIG. 42B shows the fold of expansion of TCRVγ9⁺γδ T cells from four AML patients PBMCs. FIG. 42C shows TCR Vγ9⁺γδ T cells from LC patients PBMCs exhibit more activated phenotype. Scatter dot plot graphs shows the frequency of Vγ9⁺γδ T cells, from fresh PBMCs, positive for naïve (CD27⁺CD45RA⁺), Central Memory (T_{CM}: CD27⁺ CD45RA⁻), Effector Memory (T_{EM}: CD27⁻CD45RA⁻) and Effector Memory cells that re-express CD45RA (EMRA: CD27⁻CD45RA⁺) phenotype. Each dot represented data from an a Lung Cancer patient sample. FIG. 42D shows Vγ9/CD123 bispecific effectively mediates AML γδ T cells cytotoxicity against Kasumi-3 cells. The representative graphs show the frequency of target (kasumi-3) cell lysis (%7-AAD⁺ cells) mediated by Vγ9/CD123 and Vγ9/Null bispecific antibodies respectively, upon co-culture of day 14 Zol cultured healthy (left) or AML patient PBMCs (middle and right) with target cells for 16 hours. No bispecific control well values were subtracted from bispecific wells.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An antibody that binds T Cell Receptor Gamma Variable 9 (TRGV9) comprising:
(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:104; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:105;
(2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:113; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:114;
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:123; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:124; or
(4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134.

2. The antibody of claim 1, wherein
(a) the VH CDR1, VH CDR2, VH CDR3, VL CDR1, VL CDR2, and VL CDR3 amino acid sequences are according to the Kabat numbering system;
(b) the VH CDR1, VH CDR2, VH CDR3, VL CDR1, VL CDR2, and VL CDR3 amino acid sequences are according to the Chothia numbering system;
(c) the VH CDR1, VH CDR2, VH CDR3, VL CDR1, VL CDR2, and VL CDR3 amino acid sequences are according to the AbM numbering system;
(d) the VH CDR1, VH CDR2, VH CDR3, VL CDR1, VL CDR2, and VL CDR3 amino acid sequences are according to the Contact numbering system;
(e) the VH CDR1, VH CDR2, VH CDR3, VL CDR1, VL CDR2, and VL CDR3 amino acid sequences are according to the IMGT numbering system; or
(f) wherein the VH CDR1, VH CDR2, VH CDR3, VL CDR1, VL CDR2, and VL CDR3 amino acid sequences are according to the Exemplary numbering system.

3.-7. (canceled)

8. The antibody of claim 1, wherein the antibody:
(a) is a humanized antibody;
(b) is an IgG antibody;
(c) is an IgG1, IgG2, IgG3, or IgG4 antibody;
(d) comprises a kappa light chain;
(e) comprises a lambda light chain;
(f) is a monoclonal antibody;
(g) is multivalent and/or
(h) is a multispecific antibody.

9.-24. (canceled)

25. A multispecific TRGV9 antibody, comprising
(a) a first binding domain that binds to TRGV9, wherein the first binding domain comprises a TRGV9 antibody of claim 1, and
(b) a second binding domain that binds to a second target that is not TRGV9.

26. The multispecific TRGV9 antibody of claim 25, wherein the antibody is a bispecific antibody, trispecific antibody, or quadraspecific antibody.

27.-33. (canceled)

34. The multispecific TRGV9 antibody of claim 25, wherein the TRGV9 is present on the surface of a T cell, and/or wherein the second target is present on the surface of a target cell.

35.-38. (canceled)

39. The multispecific TRGV9 antibody of claim 25, wherein:
(a) the second target is CD123;
wherein optionally the second binding domain that binds CD123 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:15; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:16;
(b) the second target is CD33;
wherein optionally the second binding domain that binds CD33 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:43; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:44;
(c) the second target is TRBC1;
wherein optionally the second binding domain that binds TRBC1 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:55; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:56;
(d) the second target is BCMA;
wherein optionally the second binding domain that binds BCMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:143; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:144; or
(e) the second target is PSMA;
wherein optionally the second binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:775; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:776.

40. (canceled)

41. A multispecific TRGV9 antibody, comprising
(a) a first binding domain that binds to TRGV9, wherein the first binding domain comprises:
(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:7; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8;
(2) (i) a VH CDR1, a VH CDR2, and a VH CDR3 having an amino acid sequence of a VH CDR1, a VH CDR2, and a VH CDR3, respectively, of a VH having an amino acid sequence of SEQ ID NO:34; and (ii) a light chain variable region (VL) comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8;
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:35; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8;
(4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:36; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:8;
(5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66;
(6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68; or
(7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96; and
(b) a second binding domain that binds to a second target that is CD33, TRBC1, BCMA or PSMA.

42. The multispecific TRGV9 antibody of claim 41, wherein:
(a) the second target is CD33;
wherein optionally the second binding arm that binds CD33 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:43; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:44;
(b) the second target is TRBC1;
wherein optionally the second binding arm that binds TRBC1 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:55; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:56;
(c) the second target is BCMA;
wherein optionally the second binding arm that binds BCMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:143; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:144; or
(d) the second target is PSMA;
wherein optionally the second binding arm that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:775; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:776.

43.-49. (canceled)

50. The multispecific TRGV9 antibody of claim 39, wherein:
(a) the VH CDR1, VH CDR2, VH CDR3, VL CDR1, VL CDR2, and VL CDR3 amino acid sequences of the second binding domain are according to the Kabat numbering system;
(b) the VH CDR1, VH CDR2, VH CDR3, VL CDR1, VL CDR2, and VL CDR3 amino acid sequences of the second binding domain are according to the Chothia numbering system;
(c) the VH CDR1, VH CDR2, VH CDR3, VL CDR1, VL CDR2, and VL CDR3 amino acid sequences of the second binding domain are according to the AbM numbering system;
(d) the VH CDR1, VH CDR2, VH CDR3, VL CDR1, VL CDR2, and VL CDR3 amino acid sequences of the second binding domain are according to the Contact numbering system;
(e) the VH CDR1, VH CDR2, VH CDR3, VL CDR1, VL CDR2, and VL CDR3 amino acid sequences of the second binding domain are according to the IMGT numbering system; or
(f) wherein the VH CDR1, VH CDR2, VH CDR3, VL CDR1, VL CDR2, and VL CDR3 amino acid sequences of the second binding domain are according to the Exemplary numbering system.

51. The multispecific TRGV9 antibody of claim 41, wherein:
(a) the VH CDR1, VH CDR2, VH CDR3, VL CDR1, VL CDR2, and VL CDR3 amino acid sequences of the first binding domain are according to the Kabat numbering system;
(b) the VH CDR1, VH CDR2, VH CDR3, VL CDR1, VL CDR2, and VL CDR3 amino acid sequences of the first binding domain are according to the Chothia numbering system;
(c) the VH CDR1, VH CDR2, VH CDR3, VL CDR1, VL CDR2, and VL CDR3 amino acid sequences of the first binding domain are according to the AbM numbering system;
(d) the VH CDR1, VH CDR2, VH CDR3, VL CDR1, VL CDR2, and VL CDR3 amino acid sequences of the first binding domain are according to the Contact numbering system;
(e) the VH CDR1, VH CDR2, VH CDR3, VL CDR1, VL CDR2, and VL CDR3 amino acid sequences of the first binding domain are according to the IMGT numbering system; or
(f) wherein the VH CDR1, VH CDR2, VH CDR3, VL CDR1, VL CDR2, and VL CDR3 amino acid sequences of the first binding domain are according to the Exemplary numbering system.

52. The multispecific TRGV9 antibody of claim 42, wherein:
(a) the VH CDR1, VH CDR2, VH CDR3, VL CDR1, VL CDR2, and VL CDR3 amino acid sequences of the second binding domain are according to the Kabat numbering system;
(b) the VH CDR1, VH CDR2, VH CDR3, VL CDR1, VL CDR2, and VL CDR3 amino acid sequences of the second binding domain are according to the Chothia numbering system;
(c) the VH CDR1, VH CDR2, VH CDR3, VL CDR1, VL CDR2, and VL CDR3 amino acid sequences of the second binding domain are according to the AbM numbering system;
(d) the VH CDR1, VH CDR2, VH CDR3, VL CDR1, VL CDR2, and VL CDR3 amino acid sequences of the second binding domain are according to the Contact numbering system;
(e) the VH CDR1, VH CDR2, VH CDR3, VL CDR1, VL CDR2, and VL CDR3 amino acid sequences of the second binding domain are according to the IMGT numbering system; or
(f) wherein the VH CDR1, VH CDR2, VH CDR3, VL CDR1, VL CDR2, and VL CDR3 amino acid sequences of the second binding domain are according to the Exemplary numbering system.

53.-57. (canceled)

58. A nucleic acid encoding the antibody of claim 1.

59. A vector comprising the nucleic acid of claim 58.

60. A host cell comprising the vector of claim 59.

61. A kit comprising the vector of claim 59 and packaging for the same.

62. A kit comprising the antibody of claim 1 and packaging for the same.

63. A pharmaceutical composition comprising the antibody of claim 1, and a pharmaceutically acceptable carrier.

64. A method of producing the pharmaceutical composition of claim 63, comprising combining the antibody with a pharmaceutically acceptable carrier to obtain the pharmaceutical composition.

65. A method of activating a T cell expressing TRGV9, comprising contacting the T cell with the antibody of claim 1; wherein optionally the contacting results in an increase in CD69, CD25, and/or Granzyme B expression, as compared to a control T cell expressing TRGV9.

66. (canceled)

67. A process for making the multispecific TRGV9 antibody of claim 25, the process comprising: a step for performing a function of obtaining the first binding domain that binds to TRGV9 present on a T cell; a step for performing a function of obtaining the second binding domain that binds to the second target on the surface of a target cell; and a step for performing a function of providing the antibody that binds to TRGV9 present on a T cell and a second target on the surface of a target cell; wherein optionally the step for performing the function of obtaining the second binding domain that binds to the second target on the surface of a target cell is repeated n times, and further comprising n steps for performing the function of providing the first binding domain that binds to TRGV9 present on a T cell and n number of target molecules, wherein n is at least 2.

68. (canceled)

69. A method of directing a T cell expressing TRGV9 to a target cell, comprising contacting the multispecific TRGV9 antibody of claim 25 with the target cell, wherein the second target is present on the surface of the target cell, and wherein the contacting directs the T cell to the target cell.

70. A method of inhibiting the growth or proliferation of a target cell, comprising contacting the multispecific TRGV9 antibody of claim 25 with the target cell having the second target present on the surface of the target cell, wherein the contacting is in the presence of a T cell expressing the TRGV9, and wherein the contacting results in the inhibition of the growth or proliferation of the target cell.

71. A method of eliminating a target cell in a subject, comprising contacting the multispecific TRGV9 antibody of claim 25 with the target cell having the second target present on the surface of the target cell, wherein the contacting is in the presence of a T cell expressing the TRGV9, and wherein the contacting results in the elimination of the target cell.

72. A method of treating a disease in a subject, comprising administering an effective amount of the multispecific TRGV9 antibody of claim 25 to the subject, wherein the disease is caused all or in part by a target cell having the second target present on the surface of the target cell.

73.-74. (canceled)

75. The multispecific TRGV9 antibody of claim 25, wherein:
(i) the second target is present on the surface of a target cell, and wherein the target cell is a cancer cell; wherein optionally
(a) the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer;
(b) the second target is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAP1, TARP, TROP2, VEGF, or VEGF-R; and/or
(c) the second target is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, or BRCA1;
(ii) the second target is present on the surface of a target cell, and wherein the target cell is a T cell;
wherein optionally the second target is TRBC1, CDR3, CD16, CD17, CD18, CD20, CD21, CD23, CD25, CD26, CD27, CD28, CD29, CD30, CD31, CD32b, CD35, CD37, CD38, CD39, CD43, CD44, CD45, CD45RA, CD45RB, CD45RC, CD45RO, CD46, CD47, CD48, CD49a, CD49b, CD49c, CD49d, CD49e, CD49f, CD50, CD52, CD53, CD54, CD55, CD56, CD57, CD58, CD59, CD60a, CD62L, CD63, CD68, CD69, CD70, CD71, CD73, CD74, CD75S, CD80, CD81, CD82, CD84, CD85A, CD85J, CD86, CD87, CD92, CD94, CD95, CD96, CD97, CD98, CD99, CD99R, CD100, CD101, CD102, CD103, CD107a, CD107b, CD108, CD109, CD119, CD120a, CD120b, CD121a, CD121b, CD122, CD124, CD126, CD127, CD128, CD129, CD130, CD132, CD134, CD137, CD146, CD147, CD148, CD150, CD152, CD153, CD154, CD156b, CD158a, CD158b1, CD158b2, CD158e1/e2, CD158f, CD158g, CD158h, CD158i, CD158j, CD158k, CD159a, CD160, CD161, CD162, CD164, CD172g, CD178, CD181, CD182, CD183, CD184, CD185, CD186, CD191, CD192, CD193, CD194, CD195, CD196, CD197, CDw198, CDw199, CD205, CD210a, CDw210b, CD212, CD215, CD217, CD218a, CD218b, CD220, CD221, CD222, CD223, CD224, CD225, CD226, CD227, CD229, CD230, CD231, CD244, CD245, CD246, CD247, CD253, CD254, CD255, CD256, CD257, CD258, CD259, CD260, CD261, CD262, CD263, CD264, CD267, CD268, CD270, CD272, CD273, CD274, CD275, CD277, CD278, CD279, CD283, CD288, CD289, CD290, CD294, CD295, CD296, CD298, CD300a, CD300c, CD300e, CD305, CD306, CD307c, CD314, CD316, CD317, CD319, CD321, CD328, CD351, CD352, CD352, CD354, CD355, CD357, CD358, CD359, CD360, CD361, CD362, or CD363:
(iii) the second target is present on the surface of a target cell, and wherein the target cell is a B cell;
wherein optionally the second target is BCMA, CD1a, CD1b, CD1c, CD1d, CD2, CD5, CD6, CD9, CD11a, CD11b, CD11c, CD17, CD18, CD19, CD20, CD21, CD22, CD23, CD24, CD25, CD26, CD27, CD29, CD30, CD31, CD32a, CD32b, CD35, CD37, CD38, CD39, CD40, CD45, CD45RA, CD45RB, CD45RC, CD45RO, CD46, CD47, CD48, CD49b, CD49c, CD49d, CD50, CD52, CD53, CD54, CD55, CD58, CD60a, CD62L, CD63, CD68, CD69, CD70, CD72, CD73, CD74, CD75, CD75S, CD77, CD79a, CD79b, CD80, CD81, CD82, CD83, CD84, CD85E, CD85I, CD85J, CD86, CD92, CD95, CD97, CD98, CD99, CD100, CD102, CD108, CD119, CD120a, CD120b, CD121b, CD122, CD124, CD125, CD126, CD130, CD132, CD137, CD138, CD139, CD147, CD148, CD150, CD152, CD162, CD164, CD166, CD167a, CD170, CD171, CD175, CD175s, CD180, CD184, CD185, CD192, CD196, CD197, CD200, CD205, CD201a, CDw210b, CD212, CD213a1, CD213a2, CD 215, CD217, CD218a, CD218b, CD220, CD221, CD222, CD224, CD225, CD226, CD227, CD229, CD230, CD232, CD252, CD252, CD254, CD255, CD256, CD257 CD258, CD259, CD260, CD261, CD262, CD263, CD264, CD267-270, CD272, CD274, CD275, CD277, CD279, CD283, CD289, CD290, CD295, CD298, CD300, CD300c, CD305, CD306, CD307a, CD307b, CD307c, CD307d, CD307e, CD314, CD215, CD316, CD317, CD319, CD321, CD327, CD328, CD329, CD338, CD351, CD352, CD353, CD354, CD355, CD356, CD357, CD358, CD360, CD361, CD362 or CD363;
(iv) the second target is present on the surface of a target cell, and wherein the target cell is a dendritic cell;
wherein optionally the second target is CD1a, CD1b, CD1c, CD1d, CD1e, CD11b, CD11c, CD16, CD17, CD18, CD19, CD21, CD23, CD29, CD33, CD35, CD36, CD38, CD39, CD40, CD45, CD45RA, CD45RB, CD45RC, CD45RO, CD48, CD49d, CD49e, CD58, CD64a, CD68, CD73, CD74, CD80, CD81, CD83, CD84, CD85A, CD85D, CD85E, CD85G, CD85J, CD86, CD88, CD97, CD101, CD116, CD120a, CD120b, CD123, CD139, CD148, CD150, CD156b, CD157, CD167, CD168, CD169, CD170, CD171, CD172a, CD172b, CD180, CD184, CD185, CD193, CD196, CD197, CD200, CD205, CD206, CD207, CD208, CD209, CDw210b, CD213a1, CD217, CD218a, CD218b, CD220, CD221, CD222, CD227, CD229, CD230, CD232, CD244, CD252, CD256, CD257, CD258, CD265, CD270, CD271, CD272, CD273, CD274, CD275, CD276, CD277, CD283, CD286, CD288, CD289, CD290, CD295, CD298, CD300a, CD300c, CD300e, CD301, CD302, CD303, CD304, CD305, CD312, CD317, CD319, CD320, CD328, CD352, CD354, CD357, or CD361;
(v) the second target is present on the surface of a target cell, and wherein the target cell is a NK cell;
wherein optionally the second target is CD2, CD7, CD8a, CD10, CD11a, CD11b, CD11c, CDw12, CD16, CD18, CD25, CD26, CD27, CD29, CD30, CD31, CD32c, CD38, CD39, CD43, CD44, CD45, CD45RA, CD45RB, CD45RC, CD45RO, CD46, CD47, CD48, CD49a, CD49b, CD49d, CD49e, CD50, CD52, CD53, CD55, CD56, CD7, CD58, CD59, CD62L, CD63, CD69, CD81, CD82, CD84, CD85C, CD85E, CD85J, CD87, CD94, CD95, CD96, CD97, CD98, CD99, CD99R, CD100, CD119, CD120a, CD120b, CD122, CD130, CD132, CD147, CD148, CD158a, CD158b1, CD158b2, CD158d, CD158e1/e2, CD158f, CD158g, CD158h, CD158i, CD158j, CD158k, CD159a, CD159c, CD160, CD161, CD172g, CD178, CD183, CD185, CDw210b, CD212, CD217, CD218a, CD218b, CD220, CD221, CD222, CD223, CD225, CD226, CD229, CD230, CD232, CD244, CD247, CD257, CD261, CD262, CD263, CD264, CD270, CD277, CD280, CD295, CD298, CD305, CD314, CD316, CD317, CD319, CD321, CD328, CD329, CD335, CD336, CD337, CD352, CD354, CD355, CD357, CD360, CD361, or CD363;
(vi) the second target is present on the surface of a target cell, and wherein the target cell is a stem cell or stem cell precursor;
wherein optionally the second target is CD8a, CDw12, CD13, CD15, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD135, CD138, CD151, CD157, CD162, CD164, CD168, CD172a, CD173, CD174, CD175, CD175s, CD176, CD183, CD191, CD200, CD201, CD205, CD217, CD220, CD221, CD222, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, or CD350;
(vii) the second target is present on the surface of a target cell, and wherein the target cell is a monocyte or macrophage;
wherein optionally the second target is CD1a, CD1b, CD1c, CD4, CD9, CD11a, CD11b, CD11c, CD11d, CDw12, CD13, CD14, CD15, CD16, CD17, CD18, CD23, CD25, CD26, CD29, CD30, CD31, CD32a, CD32b, CD32c, CD33, CD35, CD36, CD37, CD38, CD39, CD40, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD58, CD59, CD60a, CD61, CD63, CD64a, CD65, CD66, CD68, CD69, CD72, CD74, CD75, CD75S, CD80, CD81, CD82, CD84, CD85A, CD85C, CD85D, CD85E, CD85F, CD85G, CD85I, CD85J, CD85K, CD86, CD87, CD88, CD89, CD91, CD92, CD93, CD95, CD97, CD98, CD99, CD99R, CD100, CD101, CD102, CD105, CD111, CD112, CD114, CD115, CD116, CD119, CD120a, CD120b, CD121b, CD122, CD124, CD127, CD130, CD131, CD132, CD136, CD137, CD139, CD141, CD142, CD143, CD147, CD148, CD153, CD155, CD156a, CD156b, CD156c, CD157, CD162, CD163, CD164, CD165, CD166, CD168, CD169, CD170, CD171, CD172a, CD172b, CD180, CD181, CD182, CD184, CD185, CD191, CD192, CD194, CD195, CDw198, CD24, CD205, CD206, CD209, CD210a, CDw210b, CD213a1, CD213a2, CD217, CD220, CD221, CD222, CD224, CD226, CD227, CD230, CD232, CD244, CD252, CD256, CD257, CD258, CD261, CD262, CD263, CD264, CD265, CD267, CD268, CD270, CD272, CD273, CD274, CD275, CD276, CD277, CD280, CD281, CD282, CD284, CD286, CD288, CD289, CD295, CD297, CD298, CD300a, CD300c, CD300e, CD301, CD302, CD305, CD306, CD312, CD214, CD315, CD317, CD319, CD321, CD328, CD329, CD338, CD351, CD352, CD352, CD354, CD357, CD358, CD360, CD361, or CD362;
(viii) the second target is present on the surface of a target cell, and wherein the target cell is a granulocyte;
wherein optionally the second target is CD11a, CD11b, CD11c, CDw12, CD13, CD14, CD15, CD16, CD16b, CD17, CD18, CD23, CD24, CD29, CD31, CD32a, CD32b, CD32c, CD33, CD35, CD37, CD43, CD44, CD45, CD45RB, CD45RO, CD46, CD47, CD50, CD53, CD55, CD58, CD59, CD60a, CD62L, CD63, CD64a, CD65, CD65s, CD66a, CD66b, CD66c, CD66d, CD68, CD69, CD75S, CD82, CD85A, CD85D, CD85K, CD87, CD88, CD89, CD92, CD93, CD95, CD97, CD98, CD100, CD101, CD107a, CD107b, CD114, CD116, CD119, CD120a, CD120b, CD123, CD125, CD130, CD131, CD132, CD139, CD141, CD147, CD148, CD153, CD156a, CD156b, CD157, CD162, CD170, CD171, CD172a, CD177, CD178, CD181, CD182, CD183, CD192, CD193, CD195, CD203c, CD217, CD218a, CD218b, CD220, CD221, CD222, CD230, CD232, CD244, CD256, CD257, CD258, CD261, CD262, CD263, CD264, CD268, CD270, CD274, CD275, CD281, CD282, CD289, CD290, CD294, CD295, CD298, CD302, CD305, CD312, CD314, CD321, CD328, CD329, CD352, CD354, CD360, or CD362;
(ix) the second target is present on the surface of a target cell, and wherein the target cell is a platelet;
wherein optionally the second target is CD9, CD17, CD18, CD23, CD29, CD31, CD32a, CD32b, CD36, CD41, CD42a, CD42b, CD42c, CD42d, CD43, CD46, CD47, CD62P, CD63, CD69, CD82, CD84, CD98, CD99, CD107a, CD107b, CD109, CD110, CD111, CD112, CD114, CD140a, CD141, CD147, CD148, CD151, CD165, CD194, CD226, CD295, CD298, or CD321;
(x) the second target is present on the surface of a target cell, and wherein the target cell is an erythrocyte;
wherein optionally the second target is CD35, CD36, CD44, CD47, CD49e, CD55, CD58, CD59, CD75S, CD99, CD108, CD111, CD139, CD147, CD173, CD176, CD233, CD234, CD235a, CD235b, CD236, CD236R, CD238, CD239, CD240, CD241, CD242, or CD324;
(xi) the second target is present on the surface of a target cell, and wherein the target cell is an endothelial cell;
wherein optionally the second target is CD9, CD10, CD13, CD17, CD29, CD30, CD31, CD32b, CD34, CD36, CD39, CD40, CD44, CD46, CD47, CD49b, CD49c, CD49d, CD4e, CD49f, CD50, CD51, CD54, CD5, CD58, CD61, CD62E, CD62P, CD63, CD71, CD73, CD74, CD75S, CD77, CD81, CD82, CD86, CD87, CD88, CD90, CD92, CD93, CD98, CD99, CD102, CD104, CD105, CD106, CD107a, CD107b, CD109, CD110, CD111, CD112, CD114, CD117, CD119, CD120a, CD120b, CD121a, CD123, CD130, CD133, CD138, CD140a, CD140b, CD141, CD142, CD143, CD144, CDw154, CD146, CD147, CD150, CD151, CD156b, CD157, CD166, CD171, CD173, CD175S, CD176, CD178, CD184, CD192, CD200, CD201, CD202b, CD206, CD209, CD213a1, CD217, CD218a, CD220, CD221, CD222, CD224, CD225, CD228, CD230, CD234, CD239, CD242, CD246, CD248, CD252, CD266, CD280, 295, CD297, CD299, CD309, CD321, CD322, or CD344;
(xii) the second target is present on the surface of a target cell, and wherein the target cell is an epithelial cell;
wherein optionally the second target is CD1d, CD9, CD13, CD18, CD21, CD23, CD24, CD26, CD29, CD39, CD40, CD44, CD46, CD47, CD49b, CD49c, CD49e, CD49f, CD52, CD55, CD58, CD66a, CD66c, CD66e, CD66f, CD73, CD74, CD75S, CD77, CD81, CD82, CD88, 91, CD92, CD98, CD99, CD104, CD110, CD111, CD112, CD113, CD114, CD118, CD120a, CD120b, CD124, CD129, CD133, CD136, CD137, CD138, CD141, CD142, CD143, CDw145, CD151, CD164, CD165, CD166, CD167a, CD171, CD174, CD175, CD175S, CD176, CD178, CD193, CD206, CD213a2, CD217, CD220, CD221, CD222, CD224, CD227, CD230, CD234, CD239, CD249, CD286, CD295, CD296, CD321, CD324, CD326, CD331, CD332, CD333, CD334, CD339, CD340, CD344, or CD350:
(xiii) the second target is a pathogen, optionally wherein the target cell is a cell having a pathogen antigen on the surface of the target cell; wherein optionally
(a) the pathogen is a virus, wherein optionally
(1) the virus is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, Paramyxoviridae, Parvoviridae, picornaviridae, polyomaviridae, Poxviridae, reoviridae, retroviridae, rhabdoviridae, or togaviridae family; or
(2) the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus;
(b) the pathogen is a bacteria, wherein optionally
the bacteria is a bacteria of a bacillus, bartonella, bordetella, borrelia, brucella, campylobacter, chlamydia, chlamydophila, clostridium, corynebacterium, enterococcus, escherichia, francisella, haemophilus, helicobacter, legionella, leptospira, listeria, mycobacterium, mycoplasma, neisseria, pseudomonas, rickettsia, salmonella, shigella, staphylococcus, streptococcus, treponema, ureaplasma, vibrio or yersinia genus;
(c) the pathogen is a parasite, wherein optionally
(1) the parasite is a protozoa, helminth, or ectoparasite; wherein optionally
(i) the protozoa is an entamoeba, giardia, leishmania, balantidium, plasmodium, or cryptosporidium;
(ii) the helminth is a trematode, cestode, acanthocephalan, or round worm; or
(iii) the ectoparasite is a arthropod; or
(d) the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru(EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

76. The method of claim 69, wherein:
(i) the second target is present on the surface of the target cell, and wherein the target cell is a cancer cell; wherein optionally
(a) the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer;
(b) the second target is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAP1, TARP, TROP2, VEGF, or VEGF-R; and/or
(c) the second target is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, or BRCA1;
(ii) the second target is present on the surface of the target cell, and wherein the target cell is a T cell;
wherein optionally the second target is TRBC1, CDR3, CD16, CD17, CD18, CD20, CD21, CD23, CD25, CD26, CD27, CD28, CD29, CD30, CD31, CD32b, CD35, CD37, CD38, CD39, CD43, CD44, CD45, CD45RA, CD45RB, CD45RC, CD45RO, CD46, CD47, CD48, CD49a, CD49b, CD49c, CD49d, CD49e, CD49f, CD50, CD52, CD53, CD54, CD55, CD56, CD57, CD58, CD59, CD60a, CD62L, CD63, CD68, CD69, CD70, CD71, CD73, CD74, CD75S, CD80, CD81, CD82, CD84, CD85A, CD85J, CD86, CD87, CD92, CD94, CD95, CD96, CD97, CD98, CD99, CD99R, CD100, CD101, CD102, CD103, CD107a, CD107b, CD108, CD109, CD119, CD120a, CD120b, CD121a, CD121b, CD122, CD124, CD126, CD127, CD128, CD129, CD130, CD132, CD134, CD137, CD146, CD147, CD148, CD150, CD152, CD153, CD154, CD156b, CD158a, CD158b1, CD158b2, CD158e1/e2, CD158f, CD158g, CD158h, CD158i, CD158j, CD158k, CD159a, CD160, CD161, CD162, CD164, CD172g, CD178, CD181, CD182, CD183, CD184, CD185, CD186, CD191, CD192, CD193, CD194, CD195, CD196, CD197, CDw198, CDw199, CD205, CD210a, CDw210b, CD212, CD215, CD217, CD218a, CD218b, CD220, CD221, CD222, CD223, CD224, CD225, CD226, CD227, CD229, CD230, CD231, CD244, CD245, CD246, CD247, CD253, CD254, CD255, CD256, CD257, CD258, CD259, CD260, CD261, CD262, CD263, CD264, CD267, CD268, CD270, CD272, CD273, CD274, CD275, CD277, CD278, CD279, CD283, CD288, CD289, CD290, CD294, CD295, CD296, CD298, CD300a, CD300c, CD300e, CD305, CD306, CD307c, CD314, CD316, CD317, CD319, CD321, CD328, CD351, CD352, CD352, CD354, CD355, CD357, CD358, CD359, CD360, CD361, CD362, or CD363;
(iii) the second target is present on the surface of the target cell, and wherein the target cell is a B cell;
wherein optionally the second target is BCMA, CD1a, CD1b, CD1c, CD1d, CD2, CD5, CD6, CD9, CD11a, CD11b, CD11c, CD17, CD18, CD19, CD20, CD21, CD22, CD23, CD24, CD25, CD26, CD27, CD29, CD30, CD31, CD32a, CD32b, CD35, CD37, CD38, CD39, CD40, CD45, CD45RA, CD45RB, CD45RC, CD45RO, CD46, CD47, CD48, CD49b, CD49c, CD49d, CD50, CD52, CD53, CD54, CD55, CD58, CD60a, CD62L, CD63, CD68, CD69, CD70, CD72, CD73, CD74, CD75, CD75S, CD77, CD79a, CD79b, CD80, CD81, CD82, CD83, CD84, CD85E, CD85I, CD85J, CD86, CD92, CD95, CD97, CD98, CD99, CD100, CD102, CD108, CD119, CD120a, CD120b, CD121b, CD122, CD124, CD125, CD126, CD130, CD132, CD137, CD138, CD139, CD147, CD148, CD150, CD152, CD162, CD164, CD166, CD167a, CD170, CD171, CD175, CD175s, CD180, CD184, CD185, CD192, CD196, CD197, CD200, CD205, CD201a, CDw210b, CD212, CD213a1, CD213a2, CD 215, CD217, CD218a, CD218b, CD220, CD221, CD222, CD224, CD225, CD226, CD227, CD229, CD230, CD232, CD252, CD252, CD254, CD255, CD256, CD257 CD258, CD259, CD260, CD261, CD262, CD263, CD264, CD267-270, CD272, CD274, CD275, CD277, CD279, CD283, CD289, CD290, CD295, CD298, CD300, CD300c, CD305, CD306, CD307a, CD307b, CD307c, CD307d, CD307e, CD314, CD215, CD316, CD317, CD319, CD321, CD327, CD328, CD329, CD338, CD351, CD352, CD353, CD354, CD355, CD356, CD357, CD358, CD360, CD361, CD362 or CD363;
(iv) the second target is present on the surface of the target cell, and wherein the target cell is a dendritic cell;
wherein optionally the second target is CD1a, CD1b, CD1c, CD1d, CD1e, CD11b, CD11c, CD16, CD17, CD18, CD19, CD21, CD23, CD29, CD33, CD35, CD36, CD38, CD39, CD40, CD45, CD45RA, CD45RB, CD45RC, CD45RO, CD48, CD49d, CD49e, CD58, CD64a, CD68, CD73, CD74, CD80, CD81, CD83, CD84, CD85A, CD85D, CD85E, CD85G, CD85J, CD86, CD88, CD97, CD101, CD116, CD120a, CD120b, CD123, CD139, CD148, CD150, CD156b, CD157, CD167, CD168, CD169, CD170, CD171, CD172a, CD172b, CD180, CD184, CD185, CD193, CD196, CD197, CD200, CD205, CD206, CD207, CD208, CD209, CDw210b, CD213a1, CD217, CD218a, CD218b, CD220, CD221, CD222, CD227, CD229, CD230, CD232, CD244, CD252, CD256, CD257, CD258, CD265, CD270, CD271, CD272, CD273, CD274, CD275, CD276, CD277, CD283, CD286, CD288, CD289, CD290, CD295, CD298, CD300a, CD300c, CD300e, CD301, CD302, CD303, CD304, CD305, CD312, CD317, CD319, CD320, CD328, CD352, CD354, CD357, or CD361;
(v) the second target is present on the surface of the target cell, and wherein the target cell is a NK cell;
wherein optionally the second target is CD2, CD7, CD8a, CD10, CD11a, CD11b, CD11c, CDw12, CD16, CD18, CD25, CD26, CD27, CD29, CD30, CD31, CD32c, CD38, CD39, CD43, CD44, CD45, CD45RA, CD45RB, CD45RC, CD45RO, CD46, CD47, CD48, CD49a, CD49b, CD49d, CD49e, CD50, CD52, CD53, CD55, CD56, CD7, CD58, CD59, CD62L, CD63, CD69, CD81, CD82, CD84, CD85C, CD85E, CD85J, CD87, CD94, CD95, CD96, CD97, CD98, CD99, CD99R, CD100, CD119, CD120a, CD120b, CD122, CD130, CD132, CD147, CD148, CD158a, CD158b1, CD158b2, CD158d, CD158e1/e2, CD158f, CD158g, CD158h, CD158i, CD158j, CD158k, CD159a, CD159c, CD160, CD161, CD172g, CD178, CD183, CD185, CDw210b, CD212, CD217, CD218a, CD218b, CD220, CD221, CD222, CD223, CD225, CD226, CD229, CD230, CD232, CD244, CD247, CD257, CD261, CD262, CD263, CD264, CD270, CD277, CD280, CD295, CD298, CD305, CD314, CD316, CD317, CD319, CD321, CD328, CD329, CD335, CD336, CD337, CD352, CD354, CD355, CD357, CD360, CD361, or CD363;
(vi) the second target is present on the surface of the target cell, and wherein the target cell is a stem cell or stem cell precursor;
wherein optionally the second target is CD8a, CDw12, CD13, CD15, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD135, CD138, CD151, CD157, CD162, CD164, CD168, CD172a, CD173, CD174, CD175, CD175s, CD176, CD183, CD191, CD200, CD201, CD205, CD217, CD220, CD221, CD222, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, or CD350;
(vii) the second target is present on the surface of the target cell, and wherein the target cell is a monocyte or macrophage;
wherein optionally the second target is CD1a, CD1b, CD1c, CD4, CD9, CD11a, CD11b, CD11c, CD11d, CDw12, CD13, CD14, CD15, CD16, CD17, CD18, CD23, CD25, CD26, CD29, CD30, CD31, CD32a, CD32b, CD32c, CD33, CD35, CD36, CD37, CD38, CD39, CD40, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD58, CD59, CD60a, CD61, CD63, CD64a, CD65, CD66, CD68, CD69, CD72, CD74, CD75, CD75S, CD80, CD81, CD82, CD84, CD85A, CD85C, CD85D, CD85E, CD85F, CD85G, CD85I, CD85J, CD85K, CD86, CD87, CD88, CD89, CD91, CD92, CD93, CD95, CD97, CD98, CD99, CD99R, CD100, CD101, CD102, CD105, CD111, CD112, CD114, CD115, CD116, CD119, CD120a, CD120b, CD121b, CD122, CD124, CD127, CD130, CD131, CD132, CD136, CD137, CD139, CD141, CD142, CD143, CD147, CD148, CD153, CD155, CD156a, CD156b, CD156c, CD157, CD162, CD163, CD164, CD165, CD166, CD168, CD169, CD170, CD171, CD172a, CD172b, CD180, CD181, CD182, CD184, CD185, CD191, CD192, CD194, CD195, CDw198, CD24, CD205, CD206, CD209, CD210a, CDw210b, CD213a1, CD213a2, CD217, CD220, CD221, CD222, CD224, CD226, CD227, CD230, CD232, CD244, CD252, CD256, CD257, CD258, CD261, CD262, CD263, CD264, CD265, CD267, CD268, CD270, CD272, CD273, CD274, CD275, CD276, CD277, CD280, CD281, CD282, CD284, CD286, CD288, CD289, CD295, CD297, CD298, CD300a, CD300c, CD300e, CD301, CD302, CD305, CD306, CD312, CD214, CD315, CD317, CD319, CD321, CD328, CD329, CD338, CD351, CD352, CD352, CD354, CD357, CD358, CD360, CD361, or CD362;
(viii) the second target is present on the surface of the target cell, and wherein the target cell is a granulocyte;
wherein optionally the second target is CD11a, CD11b, CD11c, CDw12, CD13, CD14, CD15, CD16, CD16b, CD17, CD18, CD23, CD24, CD29, CD31, CD32a, CD32b, CD32c, CD33, CD35, CD37, CD43, CD44, CD45, CD45RB, CD45RO, CD46, CD47, CD50, CD53, CD55, CD58, CD59, CD60a, CD62L, CD63, CD64a, CD65, CD65s, CD66a, CD66b, CD66c, CD66d, CD68, CD69, CD75S, CD82, CD85A, CD85D, CD85K, CD87, CD88, CD89, CD92, CD93, CD95, CD97, CD98, CD100, CD101, CD107a, CD107b, CD114, CD116, CD119, CD120a, CD120b, CD123, CD125, CD130, CD131, CD132, CD139, CD141, CD147, CD148, CD153, CD156a, CD156b, CD157, CD162, CD170, CD171, CD172a, CD177, CD178, CD181, CD182, CD183, CD192, CD193, CD195, CD203c, CD217, CD218a, CD218b, CD220, CD221, CD222, CD230, CD232, CD244, CD256, CD257, CD258, CD261, CD262, CD263, CD264, CD268, CD270, CD274, CD275, CD281, CD282, CD289, CD290, CD294, CD295, CD298, CD302, CD305, CD312, CD314, CD321, CD328, CD329, CD352, CD354, CD360, or CD362;
(ix) the second target is present on the surface of the target cell, and wherein the target cell is a platelet
wherein optionally the second target is CD9, CD17, CD18, CD23, CD29, CD31, CD32a, CD32b, CD36, CD41, CD42a, CD42b, CD42c, CD42d, CD43, CD46, CD47, CD62P, CD63, CD69, CD82, CD84, CD98, CD99, CD107a, CD107b, CD109, CD110, CD111, CD112, CD114, CD140a, CD141, CD147, CD148, CD151, CD165, CD194, CD226, CD295, CD298, or CD321;
(x) the second target is present on the surface of the target cell, and wherein the target cell is an erythrocyte;
wherein optionally the second target is CD35, CD36, CD44, CD47, CD49e, CD55, CD58, CD59, CD75S, CD99, CD108, CD111, CD139, CD147, CD173, CD176, CD233, CD234, CD235a, CD235b, CD236, CD236R, CD238, CD239, CD240, CD241, CD242, or CD324;
(xi) the second target is present on the surface of the target cell, and wherein the target cell is is an endothelial cell;
wherein optionally the second target is CD9, CD10, CD13, CD17, CD29, CD30, CD31, CD32b, CD34, CD36, CD39, CD40, CD44, CD46, CD47, CD49b, CD49c, CD49d, CD4e, CD49f, CD50, CD51, CD54, CD5, CD58, CD61, CD62E, CD62P, CD63, CD71, CD73, CD74, CD75S, CD77, CD81, CD82, CD86, CD87, CD88, CD90, CD92, CD93, CD98, CD99, CD102, CD104, CD105, CD106, CD107a, CD107b, CD109, CD110, CD111, CD112, CD114, CD117, CD119, CD120a, CD120b, CD121a, CD123, CD130, CD133, CD138, CD140a, CD140b, CD141, CD142, CD143, CD144, CDw154, CD146, CD147, CD150, CD151, CD156b, CD157, CD166, CD171, CD173, CD175S, CD176, CD178, CD184, CD192, CD200, CD201, CD202b, CD206, CD209, CD213a1, CD217, CD218a, CD220, CD221, CD222, CD224, CD225, CD228, CD230, CD234, CD239, CD242, CD246, CD248, CD252, CD266, CD280, 295, CD297, CD299, CD309, CD321, CD322, or CD344;
(xii) the second target is present on the surface of the target cell, and wherein the target cell is an epithelial cell;
wherein optionally the second target is CD1d, CD9, CD13, CD18, CD21, CD23, CD24, CD26, CD29, CD39, CD40, CD44, CD46, CD47, CD49b, CD49c, CD49e, CD49f, CD52, CD55, CD58, CD66a, CD66c, CD66e, CD66f, CD73, CD74, CD75S, CD77, CD81, CD82, CD88, 91, CD92, CD98, CD99, CD104, CD110, CD111, CD112, CD113, CD114, CD118, CD120a, CD120b, CD124, CD129, CD133, CD136, CD137, CD138, CD141, CD142, CD143, CDw145, CD151, CD164, CD165, CD166, CD167a, CD171, CD174, CD175, CD175S, CD176, CD178, CD193, CD206, CD213a2, CD217, CD220, CD221, CD222, CD224, CD227, CD230, CD234, CD239, CD249, CD286, CD295, CD296, CD321, CD324, CD326, CD331, CD332, CD333, CD334, CD339, CD340, CD344, or CD350:
(xiii) the second target is a pathogen, optionally wherein the target cell is a cell having a pathogen antigen on the surface of the target cell; wherein optionally
(a) the pathogen is a virus, wherein optionally
(1) the virus is a virus of the adenoviridae, arenaviridae, astroviridae, bunyaviridae, caliciviridae, coronaviridae, Filoviridae, flaviviridae, hepadnaviridae, hepeviridae, orthomyxoviridae, papillomaviridae, paramyxoviridae, parvoviridae, picornaviridae, polyomaviridae, Poxviridae, reoviridae, retroviridae, rhabdoviridae, or togaviridae family; or
(2) the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus;
(b) the pathogen is a bacteria, wherein optionally
the bacteria is a bacteria of a bacillus, bartonella, bordetella, borrelia, brucella, campylobacter, chlamydia, chlamydophila, clostridium, corynebacterium, enterococcus, escherichia, francisella, haemophilus, helicobacter, legionella, leptospira, listeria, mycobacterium, mycoplasma, neisseria, pseudomonas, rickettsia, salmonella, shigella, staphylococcus, streptococcus, treponema, ureaplasma, vibrio or yersinia genus;
(c) the pathogen is a parasite, wherein optionally
(1) the parasite is a protozoa, helminth, or ectoparasite; wherein optionally
(i) the protozoa is an entamoeba, giardia, leishmania, balantidium, plasmodium, or cryptosporidium;
(ii) the helminth is a trematode, cestode, acanthocephalan, or round worm; or
(iii) the ectoparasite is a arthropod; or
(d) the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru(EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

77.-81. (canceled)

82. The method of claim 72, wherein:
(i) the second target is present on the surface of the target cell, and wherein the target cell is a cancer cell; wherein optionally
(a) the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer;
(b) the second target is angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAP1, TARP, TROP2, VEGF, or VEGF-R; and/or
(c) the second target is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, or BRCA1;
(ii) the second target is present on the surface of the target cell, and wherein the target cell is a T cell;
wherein optionally the second target is TRBC1, CDR3, CD16, CD17, CD18, CD20, CD21, CD23, CD25, CD26, CD27, CD28, CD29, CD30, CD31, CD32b, CD35, CD37, CD38, CD39, CD43, CD44, CD45, CD45RA, CD45RB, CD45RC, CD45RO, CD46, CD47, CD48, CD49a, CD49b, CD49c, CD49d, CD49e, CD49f, CD50, CD52, CD53, CD54, CD55, CD56, CD57, CD58, CD59, CD60a, CD62L, CD63, CD68, CD69, CD70, CD71, CD73, CD74, CD75S, CD80, CD81, CD82, CD84, CD85A, CD85J, CD86, CD87, CD92, CD94, CD95, CD96, CD97, CD98, CD99, CD99R, CD100, CD101, CD102, CD103, CD107a, CD107b, CD108, CD109, CD119, CD120a, CD120b, CD121a, CD121b, CD122, CD124, CD126, CD127, CD128, CD129, CD130, CD132, CD134, CD137, CD146, CD147, CD148, CD150, CD152, CD153, CD154, CD156b, CD158a, CD158b1, CD158b2, CD158e1/e2, CD158f, CD158g, CD158h, CD158i, CD158j, CD158k, CD159a, CD160, CD161, CD162, CD164, CD172g, CD178, CD181, CD182, CD183, CD184, CD185, CD186, CD191, CD192, CD193, CD194, CD195, CD196, CD197, CDw198, CDw199, CD205, CD210a, CDw210b, CD212, CD215, CD217, CD218a, CD218b, CD220, CD221, CD222, CD223, CD224, CD225, CD226, CD227, CD229, CD230, CD231, CD244, CD245, CD246, CD247, CD253, CD254, CD255, CD256, CD257, CD258, CD259, CD260, CD261, CD262, CD263, CD264, CD267, CD268, CD270, CD272, CD273, CD274, CD275, CD277, CD278, CD279, CD283, CD288, CD289, CD290, CD294, CD295, CD296, CD298, CD300a, CD300c, CD300e, CD305, CD306, CD307c, CD314, CD316, CD317, CD319, CD321, CD328, CD351, CD352, CD352, CD354, CD355, CD357, CD358, CD359, CD360, CD361, CD362, or CD363;
(iii) the second target is present on the surface of the target cell, and wherein the target cell is a B cell;
wherein optionally the second target is BCMA, CD1a, CD1b, CD1c, CD1d, CD2, CD5, CD6, CD9, CD11a, CD11b, CD11c, CD17, CD18, CD19, CD20, CD21, CD22, CD23, CD24, CD25, CD26, CD27, CD29, CD30, CD31, CD32a, CD32b, CD35, CD37, CD38, CD39, CD40, CD45, CD45RA, CD45RB, CD45RC, CD45RO, CD46, CD47, CD48, CD49b, CD49c, CD49d, CD50, CD52, CD53, CD54, CD55, CD58, CD60a, CD62L, CD63, CD68, CD69, CD70, CD72, CD73, CD74, CD75, CD75S, CD77, CD79a, CD79b, CD80, CD81, CD82, CD83, CD84, CD85E, CD85I, CD85J, CD86, CD92, CD95, CD97, CD98, CD99, CD100, CD102, CD108, CD119, CD120a, CD120b, CD121b, CD122, CD124, CD125, CD126, CD130, CD132, CD137, CD138, CD139, CD147, CD148, CD150, CD152, CD162, CD164, CD166, CD167a, CD170, CD171, CD175, CD175s, CD180, CD184, CD185, CD192, CD196, CD197, CD200, CD205, CD201a, CDw210b, CD212, CD213a1, CD213a2, CD 215, CD217, CD218a, CD218b, CD220, CD221, CD222, CD224, CD225, CD226, CD227, CD229, CD230, CD232, CD252, CD252, CD254, CD255, CD256, CD257 CD258, CD259, CD260, CD261, CD262, CD263, CD264, CD267-270, CD272, CD274, CD275, CD277, CD279, CD283, CD289, CD290, CD295, CD298, CD300, CD300c, CD305, CD306, CD307a, CD307b, CD307c, CD307d, CD307e, CD314, CD215, CD316, CD317, CD319, CD321, CD327, CD328, CD329, CD338, CD351, CD352, CD353, CD354, CD355, CD356, CD357, CD358, CD360, CD361, CD362 or CD363;
(iv) the second target is present on the surface of the target cell, and wherein the target cell is a dendritic cell;
wherein optionally the second target is CD1a, CD1b, CD1c, CD1d, CD1e, CD11b, CD11c, CD16, CD17, CD18, CD19, CD21, CD23, CD29, CD33, CD35, CD36, CD38, CD39, CD40, CD45, CD45RA, CD45RB, CD45RC, CD45RO, CD48, CD49d, CD49e, CD58, CD64a, CD68, CD73, CD74, CD80, CD81, CD83, CD84, CD85A, CD85D, CD85E, CD85G, CD85J, CD86, CD88, CD97, CD101, CD116, CD120a, CD120b, CD123, CD139, CD148, CD150, CD156b, CD157, CD167, CD168, CD169, CD170, CD171, CD172a, CD172b, CD180, CD184, CD185, CD193, CD196, CD197, CD200, CD205, CD206, CD207, CD208, CD209, CDw210b, CD213a1, CD217, CD218a, CD218b, CD220, CD221, CD222, CD227, CD229, CD230, CD232, CD244, CD252, CD256, CD257, CD258, CD265, CD270, CD271, CD272, CD273, CD274, CD275, CD276, CD277, CD283, CD286, CD288, CD289, CD290, CD295, CD298, CD300a, CD300c, CD300e, CD301, CD302, CD303, CD304, CD305, CD312, CD317, CD319, CD320, CD328, CD352, CD354, CD357, or CD361;
(v) the second target is present on the surface of the target cell, and wherein the target cell is a NK cell;
wherein optionally the second target is CD2, CD7, CD8a, CD10, CD11a, CD11b, CD11c, CDw12, CD16, CD18, CD25, CD26, CD27, CD29, CD30, CD31, CD32c, CD38, CD39, CD43, CD44, CD45, CD45RA, CD45RB, CD45RC, CD45RO, CD46, CD47, CD48, CD49a, CD49b, CD49d, CD49e, CD50, CD52, CD53, CD55, CD56, CD7, CD58, CD59, CD62L, CD63, CD69, CD81, CD82, CD84, CD85C, CD85E, CD85J, CD87, CD94, CD95, CD96, CD97, CD98, CD99, CD99R, CD100, CD119, CD120a, CD120b, CD122, CD130, CD132, CD147, CD148, CD158a, CD158b1, CD158b2, CD158d, CD158e1/e2, CD158f, CD158g, CD158h, CD158i, CD158j, CD158k, CD159a, CD159c, CD160, CD161, CD172g, CD178, CD183, CD185, CDw210b, CD212, CD217, CD218a, CD218b, CD220, CD221, CD222, CD223, CD225, CD226, CD229, CD230, CD232, CD244, CD247, CD257, CD261, CD262, CD263, CD264, CD270, CD277, CD280, CD295, CD298, CD305, CD314, CD316, CD317, CD319, CD321, CD328, CD329, CD335, CD336, CD337, CD352, CD354, CD355, CD357, CD360, CD361, or CD363;
(vi) the second target is present on the surface of the target cell, and wherein the target cell is a stem cell or stem cell precursor;
wherein optionally the second target is CD8a, CDw12, CD13, CD15, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD135, CD138, CD151, CD157, CD162, CD164, CD168, CD172a, CD173, CD174, CD175, CD175s, CD176, CD183, CD191, CD200, CD201, CD205, CD217, CD220, CD221, CD222, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, or CD350;
(vii) the second target is present on the surface of the target cell, and wherein the target cell is a monocyte or macrophage;
wherein optionally the second target is CD1a, CD1b, CD1c, CD4, CD9, CD11a, CD11b, CD11c, CD11d, CDw12, CD13, CD14, CD15, CD16, CD17, CD18, CD23, CD25, CD26, CD29, CD30, CD31, CD32a, CD32b, CD32c, CD33, CD35, CD36, CD37, CD38, CD39, CD40, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD58, CD59, CD60a, CD61, CD63, CD64a, CD65, CD66, CD68, CD69, CD72, CD74, CD75, CD75S, CD80, CD81, CD82, CD84, CD85A, CD85C, CD85D, CD85E, CD85F, CD85G, CD85I, CD85J, CD85K, CD86, CD87, CD88, CD89, CD91, CD92, CD93, CD95, CD97, CD98, CD99, CD99R, CD100, CD101, CD102, CD105, CD111, CD112, CD114, CD115, CD116, CD119, CD120a, CD120b, CD121b, CD122, CD124, CD127, CD130, CD131, CD132, CD136, CD137, CD139, CD141, CD142, CD143, CD147, CD148, CD153, CD155, CD156a, CD156b, CD156c, CD157, CD162, CD163, CD164, CD165, CD166, CD168, CD169, CD170, CD171, CD172a, CD172b, CD180, CD181, CD182, CD184, CD185, CD191, CD192, CD194, CD195, CDw198, CD24, CD205, CD206, CD209, CD210a, CDw210b, CD213a1, CD213a2, CD217, CD220, CD221, CD222, CD224, CD226, CD227, CD230, CD232, CD244, CD252, CD256, CD257, CD258, CD261, CD262, CD263, CD264, CD265, CD267, CD268, CD270, CD272, CD273, CD274, CD275, CD276, CD277, CD280, CD281, CD282, CD284, CD286, CD288, CD289, CD295, CD297, CD298, CD300a, CD300c, CD300e, CD301, CD302, CD305, CD306, CD312, CD214, CD315, CD317, CD319, CD321, CD328, CD329, CD338, CD351, CD352, CD352, CD354, CD357, CD358, CD360, CD361, or CD362;
(viii) the second target is present on the surface of the target cell, and wherein the target cell is a granulocyte;
wherein optionally the second target is CD11a, CD11b, CD11c, CDw12, CD13, CD14, CD15, CD16, CD16b, CD17, CD18, CD23, CD24, CD29, CD31, CD32a, CD32b, CD32c, CD33, CD35, CD37, CD43, CD44, CD45, CD45RB, CD45RO, CD46, CD47, CD50, CD53, CD55, CD58, CD59, CD60a, CD62L, CD63, CD64a, CD65, CD65s, CD66a, CD66b, CD66c, CD66d, CD68, CD69, CD75S, CD82, CD85A, CD85D, CD85K, CD87, CD88, CD89, CD92, CD93, CD95, CD97, CD98, CD100, CD101, CD107a, CD107b, CD114, CD116, CD119, CD120a, CD120b, CD123, CD125, CD130, CD131, CD132, CD139, CD141, CD147, CD148, CD153, CD156a, CD156b, CD157, CD162, CD170, CD171, CD172a, CD177, CD178, CD181, CD182, CD183, CD192, CD193, CD195, CD203c, CD217, CD218a, CD218b, CD220, CD221, CD222, CD230, CD232, CD244, CD256, CD257, CD258, CD261, CD262, CD263, CD264, CD268, CD270, CD274, CD275, CD281, CD282, CD289, CD290, CD294, CD295, CD298, CD302, CD305, CD312, CD314, CD321, CD328, CD329, CD352, CD354, CD360, or CD362;
(ix) the second target is present on the surface of the target cell, and wherein the target cell is a platelet
wherein optionally the second target is CD9, CD17, CD18, CD23, CD29, CD31, CD32a, CD32b, CD36, CD41, CD42a, CD42b, CD42c, CD42d, CD43, CD46, CD47, CD62P, CD63, CD69, CD82, CD84, CD98, CD99, CD107a, CD107b, CD109, CD110, CD111, CD112, CD114, CD140a, CD141, CD147, CD148, CD151, CD165, CD194, CD226, CD295, CD298, or CD321;
(x) the second target is present on the surface of the target cell, and wherein the target cell is an erythrocyte;
wherein optionally the second target is CD35, CD36, CD44, CD47, CD49e, CD55, CD58, CD59, CD75S, CD99, CD108, CD111, CD139, CD147, CD173, CD176, CD233, CD234, CD235a, CD235b, CD236, CD236R, CD238, CD239, CD240, CD241, CD242, or CD324;
(xi) the second target is present on the surface of the target cell, and wherein the target cell is an endothelial cell;
wherein optionally the second target is CD9, CD10, CD13, CD17, CD29, CD30, CD31, CD32b, CD34, CD36, CD39, CD40, CD44, CD46, CD47, CD49b, CD49c, CD49d, CD4e, CD49f, CD50, CD51, CD54, CD5, CD58, CD61, CD62E, CD62P, CD63, CD71, CD73, CD74, CD75S, CD77, CD81, CD82, CD86, CD87, CD88, CD90, CD92, CD93, CD98, CD99, CD102, CD104, CD105, CD106, CD107a, CD107b, CD109, CD110, CD111, CD112, CD114, CD117, CD119, CD120a, CD120b, CD121a, CD123, CD130, CD133, CD138, CD140a, CD140b, CD141, CD142, CD143, CD144, CDw154, CD146, CD147, CD150, CD151, CD156b, CD157, CD166, CD171, CD173, CD175S, CD176, CD178, CD184, CD192, CD200, CD201, CD202b, CD206, CD209, CD213a1, CD217, CD218a, CD220, CD221, CD222, CD224, CD225, CD228, CD230, CD234, CD239, CD242, CD246, CD248, CD252, CD266, CD280, 295, CD297, CD299, CD309, CD321, CD322, or CD344;
(xii) the second target is present on the surface of the target cell, and wherein the target cell is an epithelial cell;
wherein optionally the second target is CD1d, CD9, CD13, CD18, CD21, CD23, CD24, CD26, CD29, CD39, CD40, CD44, CD46, CD47, CD49b, CD49c, CD49e, CD49f, CD52, CD55, CD58, CD66a, CD66c, CD66e, CD66f, CD73, CD74, CD75S, CD77, CD81, CD82, CD88, 91, CD92, CD98, CD99, CD104, CD110, CD111, CD112, CD113, CD114, CD118, CD120a, CD120b, CD124, CD129, CD133, CD136, CD137, CD138, CD141, CD142, CD143, CDw145, CD151, CD164, CD165, CD166, CD167a, CD171, CD174, CD175, CD175S, CD176, CD178, CD193, CD206, CD213a2, CD217, CD220, CD221, CD222, CD224, CD227, CD230, CD234, CD239, CD249, CD286, CD295, CD296, CD321, CD324, CD326, CD331, CD332, CD333, CD334, CD339, CD340, CD344, or CD350:
(xiii) the second target is a pathogen, optionally wherein the target cell is a cell having a pathogen antigen on the surface of the target cell; wherein optionally
(a) the pathogen is a virus, wherein optionally
(1) the virus is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, Paramyxoviridae, Parvoviridae, picornaviridae, polyomaviridae, poxviridae, reoviridae, retroviridae, rhabdoviridae, or togaviridae family; or
(2) the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus;
(b) the pathogen is a bacteria, wherein optionally
the bacteria is a bacteria of a bacillus, bartonella, bordetella, borrelia, brucella, campylobacter, chlamydia, chlamydophila, clostridium, corynebacterium, enterococcus, escherichia, francisella, haemophilus, helicobacter, legionella, leptospira, listeria, mycobacterium, mycoplasma, neisseria, pseudomonas, rickettsia, salmonella, shigella, staphylococcus, streptococcus, treponema, ureaplasma, vibrio or yersinia genus;
(c) the pathogen is a parasite, wherein optionally
(1) the parasite is a protozoa, helminth, or ectoparasite; wherein optionally
(i) the protozoa is an entamoeba, giardia, leishmania, balantidium, plasmodium, or cryptosporidium;
(ii) the helminth is a trematode, cestode, acanthocephalan, or round worm; or
(iii) the ectoparasite is a arthropod; or
(d) the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru(EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

83.-114. (canceled)
